(19) United States
(12) Patent Application Publication
(10) Pub. No.: US 2023/0203134 A1
(43) Pub. Date: Jun. 29, 2023

(54) POTENT NEUTRALIZING ANTIBODIES AGAINST SARS-COV-2, GENERATION AND USES THEREOF

(71) Applicant: The Trustees of Columbia University in the City of New York, New York, NY (US)

(72) Inventors: David D. HO, Bedford, NY (US); Yaoxing HUANG, Brooklyn, NY (US); Lihong LIU, Shanghai (CN); Manoj S. NAIR, Fair Lawn, NJ (US); Pengfei WANG, Elmhurst, NY (US); Jian YU, New Providence, NJ (US); Yang LUO, New York, NY (US)

(21) Appl. No.: 17/999,387

(22) PCT Filed: May 20, 2021
(86) PCT No.: PCT/US2021/033512

§ 371 Date: Nov. 18, 2022

Related U.S. Application Data
(60) Provisional application No. 63/165,729, filed on Mar. 24, 2021.
(60) Provisional application No. 63/123,767, filed on Dec. 10, 2020.
(60) Provisional application No. 63/117,908, filed on Nov. 24, 2020.
(60) Provisional application No. 63/063,106, filed on Aug. 7, 2020.
(60) Provisional application No. 63/060,116, filed on Aug. 2, 2020.
(60) Provisional application No. 63/039,977, filed on Jun. 16, 2020.
(60) Provisional application No. 63/032,518, filed on May 29, 2020.
(60) Provisional application No. 63/027,935, filed on May 20, 2020.

(51) Int. Cl. C07K 16/10; A61P 31/14

(52) U.S. Cl. CPC C07K 16/10; A61P 31/14; A61K 2039/505

(57) ABSTRACT

The subject matter described herein relates to potent monoclonal and bispecific antibodies capable of neutralizing a SARS-CoV-2 viruses and methods of generating the antibodies.

This application claims the benefit of and priority to U.S. Provisional Patent Application No. 63/027,935, filed on May 20, 2020, U.S. Provisional Patent Application No. 63/032,518, filed on May 29, 2020, U.S. Provisional Patent Application No. 63/039,977, filed on Jun. 16, 2020, U.S. Provisional Patent Application No. 63/063,106, filed Aug. 7, 2020, U.S. Provisional Patent Application No. 63/123,767, filed Dec. 10, 2020, U.S. Provisional Patent Application No. 63/060,116, filed on Aug. 2, 2020, U.S. Provisional Patent Application No. 63/117,908, filed on Nov. 24, 2020, and U.S. Provisional Patent Application No. 63/165,729, filed on Mar. 24, 2021, the contents of each of which is hereby incorporated by reference in its entirety.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

Coronavirus disease 2019 (COVID-19) is an infectious disease caused by the severe acute respiratory syndrome coronavirus 2 (SARS-CoV-2). The disease affects multiple organs in the body including the lungs. There is an urgent need for the development of therapeutics to combat COVID-19.

### SUMMARY OF THE INVENTION

In certain aspects, the invention provides a bispecific molecule comprising a first polypeptide chain and a second polypeptide chain, wherein: the first polypeptide chain comprises: a first light chain comprising a variable domain and a constant domain; a first heavy chain comprising a variable domain and a constant domain; the second polypeptide chain comprises: a second light chain comprising a variable domain and a constant domain; and a second heavy chain comprising a variable domain and a constant domain.

In some embodiments, the first polypeptide chain and the second polypeptide chain are covalently bonded to one another. In some embodiments, the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 59 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 63. In some embodiments, the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 60 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 64. In some embodiments, the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 61 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 65. In some embodiments, the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 62 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 66.

In some embodiments, the first heavy chain comprises SEQ ID NO: 59 or SEQ ID NO: 63. In some embodiments, the first light chain comprises SEQ ID NO: 60 or SEQ ID NO: 64. In some embodiments, the second heavy chain comprises SEQ ID NO: 61 or SEQ ID NO: 65. In some embodiments, the second light chain comprises SEQ ID NO: 62 or SEQ ID NO: 66.

In some embodiments, the variable domain of the first heavy chain comprises CDR1, CDR2, and CDR3 of SEQ ID NO: 59 or CDR1, CDR2, and CDR3 of SEQ ID NO: 63. In some embodiments, the variable domain of the first light chain comprises CDR1, CDR2, and CDR3 of SEQ ID NO: 60 or CDR1, CDR2, and CDR3 of SEQ ID NO: 64. In some embodiments, the variable domain of the second heavy chain comprises CDR1, CDR2, and CDR3 of SEQ ID NO: 61 or CDR1, CDR2, and CDR3 of SEQ ID NO: 65. In some embodiments, the variable domain of the second light chain comprises CDR1, CDR2, and CDR3 SEQ ID NO: 62 or CDR1, CDR2, and CDR3 of SEQ ID NO: 66.

In some embodiments, the variable domain of the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27. In some embodiments, the variable domain of the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28. In some embodiments, the variable domain of the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13. In some embodiments, the variable domain of the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14.

In some embodiments, the variable domain of the first heavy chain comprises SEQ ID NO: 13 or SEQ ID NO: 27. In some embodiments, the variable domain of the first light chain comprises SEQ ID NO: 14 or SEQ ID NO: 28. In some embodiments, the variable domain of the second heavy chain comprises SEQ ID NO: 27 or SEQ ID NO: 13. In some embodiments, the variable domain of the second light chain comprises SEQ ID NO: 28 or SEQ ID NO: 14.

In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 59, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 60, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 61, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 62. In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 63, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 64, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 65, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 66. In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 13, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28.

In some embodiments, the molecule comprises SEQ ID NOs: 59, 60, 61, and 62. In some embodiments, the molecule comprises SEQ ID NOs: 63, 64, 65, and 66. In some embodiments, the molecule comprises SEQ ID NOs: 13, 14, 27, and 28.

In some embodiments, the molecule further comprises one or more disulfide bonds. In some embodiments, the molecule further comprises one or more linker sequences. In some embodiments, the molecule is capable of neutralizing a coronavirus. In some embodiments, the coronavirus is a SARS-CoV-2 virus strain. In some embodiments, the molecule specifically recognizes a first epitope and a second epitope on a SARS-CoV-2 particle. In some embodiments, the first epitope comprises at least a portion of a receptor binding domain (RBD) on an S protein of a SARS-CoV-2 particle. In some embodiments, the second epitope comprises at least a portion of a N-terminal domain (NTD) on an S protein of a SARS-CoV-2 particle.

In certain aspects, the invention provides a method of treating a COVID-19 infection in a subject in need thereof, the method comprising administering to the subject a composition comprising a bispecific molecule, wherein the bispecific molecule comprises a first polypeptide chain and a second polypeptide chain, wherein: the first polypeptide chain comprises: a first light chain comprising a variable domain and a constant domain; a first heavy chain comprising a variable domain and a constant domain; the second polypeptide chain comprises: a second light chain comprising a variable domain and a constant domain; and a second heavy chain comprising a variable domain and a constant domain.

In some embodiments, the first polypeptide chain and the second polypeptide chain are covalently bonded to one another. In some embodiments, the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 59 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 63. In some embodiments, the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 60 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 64. In some embodiments, the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 61 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 65. In some embodiments, the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 62 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 66.

In some embodiments, the first heavy chain comprises SEQ ID NO: 59 or SEQ ID NO: 63. In some embodiments, the first light chain comprises SEQ ID NO: 60 or SEQ ID NO: 64. In some embodiments, the second heavy chain comprises SEQ ID NO: 61 or SEQ ID NO: 65. In some embodiments, the second light chain comprises SEQ ID NO: 62 or SEQ ID NO: 66. In some embodiments, the variable domain of the first heavy chain comprises CDR1, CDR2, and CDR3 of SEQ ID NO: 59 or CDR1, CDR2, and CDR3 of SEQ ID NO: 63. In some embodiments, the variable domain of the first light chain comprises CDR1, CDR2, and CDR3 of SEQ ID NO: 60 or CDR1, CDR2, and CDR3 of SEQ ID NO: 64. In some embodiments, the variable domain of the second heavy chain comprises CDR1, CDR2, and CDR3 of SEQ ID NO: 61 or CDR1, CDR2, and CDR3 of SEQ ID NO: 65. In some embodiments, the variable domain of the second light chain comprises CDR1, CDR2, and CDR3 SEQ ID NO: 62 or CDR1, CDR2, and CDR3 of SEQ ID NO: 66.

In some embodiments, the variable domain of the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27. In some embodiments, the variable domain of the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28. In some embodiments, the variable domain of the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13. In some embodiments, the variable domain of the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14.

In some embodiments, the variable domain of the first heavy chain comprises SEQ ID NO: 13 or SEQ ID NO: 27. In some embodiments, the variable domain of the first light chain comprises SEQ ID NO: 14 or SEQ ID NO: 28. In some embodiments, the variable domain of the second heavy chain comprises SEQ ID NO: 27 or SEQ ID NO: 13. In some embodiments, the variable domain of the second light chain comprises SEQ ID NO: 28 or SEQ ID NO: 14.

In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 59, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 60, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 61, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 62. In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 63, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 64, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 65, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 66. In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 13, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28.

In some embodiments, the molecule comprises SEQ ID NOs: 59, 60, 61, and 62. In some embodiments, the molecule comprises SEQ ID NOs: 63, 64, 65, and 66. In some embodiments, the molecule comprises SEQ ID NOs: 13, 14, 27, and 28.

In some embodiments, the molecule further comprises one or more disulfide bonds. In some embodiments, the molecule further comprises one or more linker sequences. In some embodiments, the molecule is capable of neutralizing a coronavirus. In some embodiments, the coronavirus is a SARS-CoV-2 virus strain. In some embodiments, the molecule specifically recognizes a first epitope and a second epitope on a SARS-CoV-2 particle. In some embodiments, the first epitope comprises at least a portion of a receptor binding domain (RBD) on a spike protein of a SARS-CoV-2 particle. In some embodiments, the second epitope comprises at least a portion of a N-terminal domain (NTD) on a spike protein of a SARS-CoV-2 particle. In some embodiments, the composition is administered in combination with a second therapeutic agent.

In certain aspects, the invention provides a method of preventing a COVID-19 infection in a subject in need thereof, the method comprising administering to the subject a composition comprising a bispecific molecule, wherein the bispecific molecule comprises a first polypeptide chain and a second polypeptide chain, wherein: the first polypeptide chain comprises: a first light chain comprising a variable domain and a constant domain; a first heavy chain comprising a variable domain and a constant domain; the second polypeptide chain comprises: a second light chain comprising a variable domain and a constant domain; and a second heavy chain comprising a variable domain and a constant domain.

In some embodiments, the first polypeptide chain and the second polypeptide chain are covalently bonded to one another. In some embodiments, the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 59 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 63. In some embodiments, the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 60 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 64. In some embodiments, the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 61 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 65. In some embodiments, the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 62 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 66.

In some embodiments, the first heavy chain comprises SEQ ID NO: 59 or SEQ ID NO: 63. In some embodiments, the first light chain comprises SEQ ID NO: 60 or SEQ ID NO: 64. In some embodiments, the second heavy chain comprises SEQ ID NO: 61 or SEQ ID NO: 65. In some embodiments, the second light chain comprises SEQ ID NO: 62 or SEQ ID NO: 66.

In some embodiments, the variable domain of the first heavy chain comprises CDR1, CDR2, and CDR3 of SEQ ID NO: 59 or CDR1, CDR2, and CDR3 of SEQ ID NO: 63. In some embodiments, the variable domain of the first light chain comprises CDR1, CDR2, and CDR3 of SEQ ID NO: 60 or CDR1, CDR2, and CDR3 of SEQ ID NO: 64. In some embodiments, the variable domain of the second heavy chain comprises CDR1, CDR2, and CDR3 of SEQ ID NO: 61 or CDR1, CDR2, and CDR3 of SEQ ID NO: 65. In some embodiments, the variable domain of the second light chain comprises CDR1, CDR2, and CDR3 SEQ ID NO: 62 or CDR1, CDR2, and CDR3 of SEQ ID NO: 66.

In some embodiments, the variable domain of the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27. In some embodiments, the variable domain of the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28. In some embodiments, the variable domain of the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13. In some embodiments, the variable domain of the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14.

In some embodiments, the variable domain of the first heavy chain comprises SEQ ID NO: 13 or SEQ ID NO: 27. In some embodiments, the variable domain of the first light chain comprises SEQ ID NO: 14 or SEQ ID NO: 28. In some embodiments, the variable domain of the second heavy chain comprises SEQ ID NO: 27 or SEQ ID NO: 13. In some embodiments, the variable domain of the second light chain comprises SEQ ID NO: 28 or SEQ ID NO: 14.

In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 59, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 60, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 61, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 62. In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 63, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 64, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 65, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 66. In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 13, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28.

In some embodiments, the molecule comprises SEQ ID NOs: 59, 60, 61, and 62. In some embodiments, the molecule comprises SEQ ID NOs: 63, 64, 65, and 66. In some embodiments, the molecule comprises SEQ ID NOs: 13, 14, 27, and 28.

In some embodiments, the molecule further comprises one or more disulfide bonds. In some embodiments, the molecule further comprises one or more linker sequences. In some embodiments, the molecule is capable of neutralizing a coronavirus. In some embodiments, the coronavirus is a SARS-CoV-2 virus strain. In some embodiments, the molecule specifically recognizes a first epitope and a second epitope on a SARS-CoV-2 particle. In some embodiments, the first epitope comprises at least a portion of a receptor binding domain (RBD) on an S protein of a SARS-CoV-2 particle. In some embodiments, the second epitope comprises at least a portion of a N-terminal domain (NTD) on an S protein of a SARS-CoV-2 particle. In some embodiments, the composition is administered in combination with a second therapeutic agent.

In certain aspects, the invention provides a synthesized monoclonal antibody, or a functional fragment thereof, comprising a heavy chain having a variable domain and a constant domain and a light chain having a variable domain and a constant domain, wherein the antibody binds an antigen on a coronavirus particle.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the heavy chain variable domain comprises SEQ ID NO: 15. In some embodiments, the light chain variable domain comprises SEQ ID NO: 16.

In some embodiments, the antibody comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15 and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the antibody comprises SEQ ID NO: 15 and SEQ ID NO: 16.

In some embodiments, the heavy chain variable domain comprises the CDR1, CDR2, and CDR3 regions of SEQ ID NO: 15. In some embodiments, the light chain variable domain comprises the CDR1, CDR2, and CDR3 regions of SEQ ID NO: 16.

In some embodiments, the antibody neutralizes a coronavirus. In some embodiments, the coronavirus is SARS-CoV-2. In some embodiments, the antibody specifically binds an epitope on the surface of the coronavirus. In some embodiments, the epitope comprises at least a portion of a coronavirus spike protein. In some embodiments, the at least a portion of a coronavirus spike protein comprises a receptor binding domain (RBD) of a spike protein. In some embodiments, the coronavirus is a SARS-CoV-2 coronavirus.

In certain aspects, the invention provides a method of treating a COVID-19 infection in a subject in need thereof, the method comprising administering to the subject a therapeutically effective amount of a synthesized monoclonal antibody, or a functional fragment thereof, comprising a heavy chain having a variable domain and a constant domain and a light chain having a variable domain and a constant domain, wherein the antibody binds an antigen on a coronavirus particle.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the heavy chain variable domain comprises SEQ ID NO: 15. In some embodiments, the light chain variable domain comprises SEQ ID NO: 16.

In some embodiments, the antibody comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15 and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the antibody comprises SEQ ID NO: 15 and SEQ ID NO: 16.

In some embodiments, the heavy chain variable domain comprises the CDR1, CDR2, and CDR3 regions of SEQ ID NO: 15. In some embodiments, the light chain variable domain comprises the CDR1, CDR2, and CDR3 regions of SEQ ID NO: 16.

In some embodiments, the antibody neutralizes a coronavirus. In some embodiments, the coronavirus is SARS-CoV-2. In some embodiments, the antibody neutralizes a coronavirus. In some embodiments, the coronavirus is SARS-CoV-2. In some embodiments, the antibody specifically binds an epitope on the surface of the coronavirus. In some embodiments, the epitope comprises at least a portion of a coronavirus spike protein. In some embodiments, the at least a portion of a coronavirus spike protein comprises a receptor binding domain (RBD) of a spike protein. In some embodiments, the coronavirus is a SARS-CoV-2 coronavirus. In some embodiments, the antibody is administered in combination with a second pharmaceutical agent.

In certain aspects, the invention provides a method of preventing a COVID-19 infection in a subject in need thereof, the method comprising administering to the subject a therapeutically effective amount of a synthesized monoclonal antibody, or a functional fragment thereof, comprising a heavy chain having a variable domain and a constant domain and a light chain having a variable and a constant domain, wherein the antibody binds an antigen on a coronavirus particle.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the heavy chain variable domain comprises SEQ ID NO: 15. In some embodiments, the light chain variable domain comprises SEQ ID NO: 16.

In some embodiments, the antibody comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15 and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the antibody comprises SEQ ID NO: 15 and SEQ ID NO: 16.

In some embodiments, the heavy chain variable domain comprises the CDR1, CDR2, and CDR3 regions of SEQ ID NO: 15. In some embodiments, the light chain variable domain comprises the CDR1, CDR2, and CDR3 regions of SEQ ID NO: 16.

In some embodiments, the antibody neutralizes a coronavirus. In some embodiments, the coronavirus is SARS-CoV-2. In some embodiments, the antibody neutralizes a coronavirus. In some embodiments, the coronavirus is SARS-CoV-2. In some embodiments, the antibody specifically binds an epitope on the surface of the coronavirus. In some embodiments, the epitope comprises at least a portion of a coronavirus spike protein. In some embodiments, the at least a portion of a coronavirus spike protein comprises a receptor binding domain (RBD) of a spike protein. In some embodiments, the coronavirus is a SARS-CoV-2 coronavirus. In some embodiments, the antibody is administered in combination with a second pharmaceutical agent.

In certain aspects, the invention provides a synthesized monoclonal antibody, or a functional fragment thereof, comprising a heavy chain having a variable domain and a constant domain and a light chain having a variable domain and a constant domain, wherein the antibody binds an antigen on a coronavirus particle.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 29, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 31.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 33, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 39, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 45.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 30, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 32.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 34, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 40, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 46.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 29 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 30.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 31 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 32. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 33 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 34. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 39 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 40. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 45 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 46.

In some embodiments, the heavy chain variable domain comprises SEQ ID NO: 3 and the light chain variable domain comprises SEQ ID NO: 4. In some embodiments, the heavy chain variable domain comprises SEQ ID NO: 7 and the light chain variable domain comprises SEQ ID NO: 8. In some embodiments, the heavy chain variable domain comprises SEQ ID NO: 9 and the light chain variable domain comprises SEQ ID NO: 10. In some embodiments, the heavy chain variable domain comprises SEQ ID NO: 11 and the light chain variable domain comprises SEQ ID NO: 12. In some embodiments, the heavy chain variable domain comprises SEQ ID NO: 17 and the light chain variable domain comprises SEQ ID NO: 18. In some embodiments, the heavy chain variable domain comprises SEQ ID NO: 19 and the light chain variable domain comprises SEQ ID NO: 20. In some embodiments, the heavy chain variable domain comprises SEQ ID NO: 21 and the light chain variable domain comprises SEQ ID NO: 22. In some embodiments, the heavy chain variable domain comprises SEQ ID NO: 25 and the light chain variable domain comprises SEQ ID NO: 26. In some embodiments, the heavy chain variable domain comprises SEQ ID NO: 29 and the light chain variable domain comprises SEQ ID NO: 30. In some embodiments, the heavy chain variable domain comprises SEQ ID NO: 31 and the light chain variable domain comprises SEQ ID NO: 32. In some embodiments, the heavy chain variable domain comprises SEQ ID NO: 33 and the light chain variable domain comprises SEQ ID NO: 34. In some embodiments, the heavy chain variable domain comprises SEQ ID NO: 37 and the light chain variable domain comprises SEQ ID NO: 38. In some embodiments, the heavy chain variable domain comprises SEQ ID NO: 39 and the light chain variable domain comprises SEQ ID NO: 40. In some embodiments, the heavy chain variable domain comprises SEQ ID NO: 43 and the light chain variable domain comprises SEQ ID NO: 44. In some embodiments, the heavy chain variable domain comprises SEQ ID NO: 45 and the light chain variable domain comprises SEQ ID NO: 46.

In some embodiments, the heavy chain variable domain comprises the CDR1, CDR2, and CDR3 regions of SEQ ID NO: 3; the CDR1, CDR2, and CDR3 regions of SEQ ID NO: 7; the CDR1, CDR2, and CDR3 regions of SEQ ID NO: 9; the CDR1, CDR2, and CDR3 regions of SEQ ID NO: 11; or the CDR1, CDR2, and CDR3 regions of SEQ ID NO: 17. In some embodiments, the heavy chain variable domain comprises the CDR1, CDR2, and CDR3 regions of SEQ ID NO: 19; the CDR1, CDR2, and CDR3 regions of SEQ ID NO: 21; the CDR1, CDR2, and CDR3 regions of SEQ ID NO: 25; the CDR1, CDR2, and CDR3 regions of SEQ ID NO: 29; or the CDR1, CDR2, and CDR3 regions of SEQ ID NO: 31. In some embodiments, the heavy chain variable domain comprises the CDR1, CDR2, and CDR3 regions of SEQ ID NO: 33; the CDR1, CDR2, and CDR3 regions of SEQ ID NO: 37; the CDR1, CDR2, and CDR3 regions of SEQ ID NO: 39; the CDR1, CDR2, and CDR3 regions of SEQ ID NO: 43; or the CDR1, CDR2, and CDR3 regions of SEQ ID NO: 45. In some embodiments, the light chain variable domain comprises the CDR1, CDR2, and CDR3 regions of SEQ ID NO: 4; the CDR1, CDR2, and CDR3 regions of SEQ ID NO: 8; the CDR1, CDR2, and CDR3 regions of SEQ ID NO: 10; the CDR1, CDR2, and CDR3 regions of SEQ ID NO: 12; or the CDR1, CDR2, and CDR3 regions of SEQ ID NO: 18. In some embodiments, the light chain variable domain comprises the CDR1, CDR2, and CDR3 regions of SEQ ID NO: 20; the CDR1, CDR2, and CDR3 regions of SEQ ID NO: 22; the CDR1, CDR2, and CDR3 regions of SEQ ID NO: 26; the CDR1, CDR2, and CDR3 regions of SEQ ID NO: 30; or the CDR1, CDR2, and CDR3 regions of SEQ ID NO: 32. In some embodiments, the light chain variable domain comprises the CDR1, CDR2, and CDR3 regions of SEQ ID NO: 34; the CDR1, CDR2, and CDR3 regions of SEQ ID NO: 38; the CDR1, CDR2, and CDR3 regions of SEQ ID NO: 40; the CDR1, CDR2, and CDR3 regions of SEQ ID NO: 44; or the CDR1, CDR2, and CDR3 regions of SEQ ID NO: 46.

In some embodiments, the antibody neutralizes a coronavirus. In some embodiments, the coronavirus is a SARS-CoV-2 virus. In some embodiments, the coronavirus is a SARS-CoV-2 variant strain. In some embodiments, the variant strain is selected from the group consisting of WA1, B.1.1.7, B1.526, B1.351, P1, B1.352, and B.1.427.

In some embodiments, the antibody specifically binds an epitope on the surface of the coronavirus. In some embodiments, the epitope comprises at least a portion of a coronavirus spike protein. In some embodiments, the at least a portion of a coronavirus spike protein comprises a receptor binding domain (RBD) of a spike protein. In some embodiments, the at least a portion of a coronavirus spike protein comprises a N-Terminal domain (NTD) of a spike protein. In some embodiments, the coronavirus is a SARS-CoV-2 coronavirus.

In certain aspects, the invention provides a method of treating or preventing a COVID-19 infection in a subject in need thereof, the method comprising administering to the subject a therapeutically effective amount of a synthesized monoclonal antibody, or a functional fragment thereof, comprising a heavy chain having a variable domain and a constant domain and a light chain having a variable domain and a constant domain, wherein the antibody binds an antigen on a coronavirus particle.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 29, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 31.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 33, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 39, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 45.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 30, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 32.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 34, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 40, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 46.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 29 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 30. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 31 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 32. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 33 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 34. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 39 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 40. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 45 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 46.

In some embodiments, the heavy chain variable domain comprises SEQ ID NO: 3 and the light chain variable domain comprises SEQ ID NO: 4. In some embodiments, the heavy chain variable domain comprises SEQ ID NO: 7 and the light chain variable domain comprises SEQ ID NO: 8. In some embodiments, the heavy chain variable domain comprises SEQ ID NO: 9 and the light chain variable domain comprises SEQ ID NO: 10. In some embodiments, the heavy chain variable domain comprises SEQ ID NO: 11 and the light chain variable domain comprises SEQ ID NO: 12. In some embodiments, the heavy chain variable domain comprises SEQ ID NO: 17 and the light chain variable domain comprises SEQ ID NO: 18. In some embodiments, the heavy chain variable domain comprises SEQ ID NO: 19 and the light chain variable domain comprises SEQ ID NO: 20. In some embodiments, the heavy chain variable domain comprises SEQ ID NO: 21 and the light chain variable domain comprises SEQ ID NO: 22. In some embodiments, the heavy chain variable domain comprises SEQ ID NO: 25 and the light chain variable domain comprises SEQ ID NO: 26. In some embodiments, the heavy chain variable domain comprises SEQ ID NO: 29 and the light chain variable domain comprises SEQ ID NO: 30. In some embodiments, the heavy chain variable domain comprises SEQ ID NO: 31 and the light chain variable domain comprises SEQ ID NO: 32. In some embodiments, the heavy chain variable domain comprises SEQ ID NO: 33 and the light chain variable domain comprises SEQ ID NO: 34. In some embodiments, the heavy chain variable domain comprises SEQ ID NO: 37 and the light chain variable domain comprises SEQ ID NO: 38. In some embodiments, the heavy chain variable domain comprises SEQ ID NO: 39 and the light chain variable domain comprises SEQ ID NO: 40. In some embodiments, the heavy chain variable domain comprises SEQ ID NO: 43 and the light chain variable domain comprises SEQ ID NO: 44. In some embodiments, the heavy chain variable domain comprises SEQ ID NO: 45 and the light chain variable domain comprises SEQ ID NO: 46.

In some embodiments, the heavy chain variable domain comprises the CDR1, CDR2, and CDR3 regions of SEQ ID NO: 3; the CDR1, CDR2, and CDR3 regions of SEQ ID NO: 7; the CDR1, CDR2, and CDR3 regions of SEQ ID NO: 9; the CDR1, CDR2, and CDR3 regions of SEQ ID NO: 11; or the CDR1, CDR2, and CDR3 regions of SEQ ID NO: 17. In some embodiments, the heavy chain variable domain comprises the CDR1, CDR2, and CDR3 regions of SEQ ID NO: 19; the CDR1, CDR2, and CDR3 regions of SEQ ID NO: 21; the CDR1, CDR2, and CDR3 regions of SEQ ID NO: 25; the CDR1, CDR2, and CDR3 regions of SEQ ID NO: 29; or the CDR1, CDR2, and CDR3 regions of SEQ ID NO: 31. In some embodiments, the heavy chain variable domain comprises the CDR1, CDR2, and CDR3 regions of SEQ ID NO: 33; the CDR1, CDR2, and CDR3 regions of SEQ ID NO: 37; the CDR1, CDR2, and CDR3 regions of SEQ ID NO: 39; the CDR1, CDR2, and CDR3 regions of SEQ ID NO: 43; or the CDR1, CDR2, and CDR3 regions of SEQ ID NO: 45. In some embodiments, the light chain variable domain comprises the CDR1, CDR2, and CDR3 regions of SEQ ID NO: 4; the CDR1, CDR2, and CDR3 regions of SEQ ID NO: 8; the CDR1, CDR2, and CDR3 regions of SEQ ID NO: 10; the CDR1, CDR2, and CDR3 regions of SEQ ID NO: 12; or the CDR1, CDR2, and CDR3 regions of SEQ ID NO: 18. In some embodiments, the light chain variable domain comprises the CDR1, CDR2, and CDR3 regions of SEQ ID NO: 20; the CDR1, CDR2, and CDR3 regions of SEQ ID NO: 22; the CDR1, CDR2, and CDR3 regions of SEQ ID NO: 26; the CDR1, CDR2, and CDR3 regions of SEQ ID NO: 30; or the CDR1, CDR2, and CDR3 regions of SEQ ID NO: 32. In some embodiments, the light chain variable domain comprises the CDR1, CDR2, and CDR3 regions of SEQ ID NO: 34; the CDR1, CDR2, and CDR3 regions of SEQ ID NO: 38; the CDR1, CDR2, and CDR3 regions of SEQ ID NO: 40; the CDR1, CDR2, and CDR3 regions of SEQ ID NO: 44; or the CDR1, CDR2, and CDR3 regions of SEQ ID NO: 46.

In some embodiments, the antibody neutralizes a coronavirus. In some embodiments, the coronavirus is a SARS-CoV-2 virus. In some embodiments, the coronavirus is a SARS-CoV-2 variant strain. In some embodiments, the variant strain is selected from the group consisting of WA1, B.1.1.7, B1.526, B1.351, P1, B1.352, and B.1.427.

In some embodiments, the antibody specifically binds an epitope on the surface of the coronavirus. In some embodiments, the epitope comprises at least a portion of a coronavirus spike protein. In some embodiments, the at least a portion of a coronavirus spike protein comprises a receptor binding domain (RBD) of a spike protein. In some embodiments, the at least a portion of a coronavirus spike protein comprises a N-Terminal domain (NTD) of a spike protein. In some embodiments, the coronavirus is a SARS-CoV-2 coronavirus. In some embodiments, the composition is administered in combination with a second therapeutic agent.

In certain aspects, the invention provides a bispecific molecule comprising a first polypeptide chain and a second polypeptide chain, wherein: the first polypeptide chain comprises: a first light chain comprising a variable domain and a constant domain; a first heavy chain comprising a variable domain and a constant domain; the second polypeptide chain comprises: a second light chain comprising a variable domain and a constant domain; and a second heavy chain comprising a variable domain and a constant domain.

In some embodiments, the first polypeptide chain and the second polypeptide chain are covalently bonded to one another. In some embodiments, the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 51, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 55, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 67, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 71, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 75, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 79, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 83, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 87, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 91, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 95, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 99, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 103, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 107, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 115, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 119, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 123, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 127, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 131, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 139, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 143, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 147, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 151, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 155, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 159, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 163, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 167, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 171, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 175, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 179, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO:183.

In some embodiments, the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 52, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 56, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 68; a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 72, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 76, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 80, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 84, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 88, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 92, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 96, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 100, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 104, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 108, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 116, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 120, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 124, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 128, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 132, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 140, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 144, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 148, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 152, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 156, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 160, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 164, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 168, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 172, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 176, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 180, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO:184.

In some embodiments, the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 53, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 57, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 69, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 73, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 77, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 81, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 85, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 89, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 93, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 97, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 101, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 105, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 109, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 117, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 121, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 125, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 129, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 133, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 141, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 145, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 149, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 153, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 157, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 161, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 165, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 169, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 173, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 177, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 181, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 185.

In some embodiments, the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 54, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 58, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 70, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 74, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 78, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 82, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 86, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 90, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 94, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 98, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 102, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 106, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 110, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 118, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 122, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 126, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 130, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 134, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 142, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 146, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 150, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 154, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 158, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 162, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 166, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 170, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 174, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 178, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 182, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 186.

In some embodiments, variable domain of the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 41.

In some embodiments, variable domain of the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 42.

In some embodiments, variable domain of the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 29, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 23, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 39, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9.

In some embodiments, variable domain of the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 30, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 24, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 40, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10.

In some embodiments, the variable domain of the first heavy chain comprises the CDR1, CDR2, and CDR3 of SEQ ID NO: 51; the CDR1, CDR2, and CDR3 of SEQ ID NO: 55; the CDR1, CDR2, and CDR3 of SEQ ID NO: 67; the CDR1, CDR2, and CDR3 of SEQ ID NO: 71; the CDR1, CDR2, and CDR3 of SEQ ID NO: 75; the CDR1, CDR2, and CDR3 of SEQ ID NO: 79; the CDR1, CDR2, and CDR3 of SEQ ID NO: 83; the CDR1, CDR2, and CDR3 of SEQ ID NO: 87; the CDR1, CDR2, and CDR3 of SEQ ID NO: 91; the CDR1, CDR2, and CDR3 of SEQ ID NO: 95; the CDR1, CDR2, and CDR3 of SEQ ID NO: 99; the CDR1, CDR2, and CDR3 of SEQ ID NO: 103; the CDR1, CDR2, and CDR3 of SEQ ID NO: 107; the CDR1, CDR2, and CDR3 of SEQ ID NO: 115; the CDR1, CDR2, and CDR3 of SEQ ID NO: 119, the CDR1, CDR2, and CDR3 of SEQ ID NO: 123, the CDR1, CDR2, and CDR3 of SEQ ID NO: 127; the CDR1, CDR2, and CDR3 of SEQ ID NO: 131; the CDR1, CDR2, and CDR3 of SEQ ID NO: 139; the CDR1, CDR2, and CDR3 of SEQ ID NO: 143; the CDR1, CDR2, and CDR3 of SEQ ID NO: 147; the CDR1, CDR2, and CDR3 of SEQ ID NO: 151; the CDR1, CDR2, and CDR3 of SEQ ID NO: 155; the CDR1, CDR2, and CDR3 of SEQ ID NO: 159; the CDR1, CDR2, and CDR3 of SEQ ID NO: 163; the CDR1, CDR2, and CDR3 of SEQ ID NO: 167, the CDR1, CDR2, and CDR3 of SEQ ID NO: 171, the CDR1, CDR2, and CDR3 of SEQ ID NO: 175, the CDR1, CDR2, and CDR3 of SEQ ID NO: 179, or the CDR1, CDR2, and CDR3 of SEQ ID NO: 183.

In some embodiments, the variable domain of the first light chain comprises the CDR1, CDR2, and CDR3 of SEQ ID NO: 52; the CDR1, CDR2, and CDR3 of SEQ ID NO: 56; the CDR1, CDR2, and CDR3 of SEQ ID NO: 68; the CDR1, CDR2, and CDR3 of SEQ ID NO: 72; the CDR1, CDR2, and CDR3 of SEQ ID NO: 76; the CDR1, CDR2, and CDR3 of SEQ ID NO: 80; the CDR1, CDR2, and CDR3 of SEQ ID NO: 84; the CDR1, CDR2, and CDR3 of SEQ ID NO: 88; the CDR1, CDR2, and CDR3 of SEQ ID NO: 92; the CDR1, CDR2, and CDR3 of SEQ ID NO: 96; the CDR1, CDR2, and CDR3 of SEQ ID NO: 100; the CDR1, CDR2, and CDR3 of SEQ ID NO: 104; the CDR1, CDR2, and CDR3 of SEQ ID NO: 108; the CDR1, CDR2, and CDR3 of SEQ ID NO: 116; the CDR1, CDR2, and CDR3 of SEQ ID NO: 120; the CDR1, CDR2, and CDR3 of SEQ ID NO: 124; the CDR1, CDR2, and CDR3 of SEQ ID NO: 128; the CDR1, CDR2, and CDR3 of SEQ ID NO: 132; the CDR1, CDR2, and CDR3 of SEQ ID NO: 140; the CDR1, CDR2, and CDR3 of SEQ ID NO: 144; the CDR1, CDR2, and CDR3 of SEQ ID NO: 148; the CDR1, CDR2, and CDR3 of SEQ ID NO: 152; the CDR1, CDR2, and CDR3 of SEQ ID NO: 156; the CDR1, CDR2, and CDR3 of SEQ ID NO: 160; the CDR1, CDR2, and CDR3 of SEQ ID NO: 164; the CDR1, CDR2, and CDR3 of SEQ ID NO: 168, the CDR1, CDR2, and CDR3 of SEQ ID NO: 172, the CDR1, CDR2, and CDR3 of SEQ ID NO: 176, the CDR1, CDR2, and CDR3 of SEQ ID NO: 180, or the CDR1, CDR2, and CDR3 of SEQ ID NO: 184.

In some embodiments, the variable domain of the second heavy chain comprises the CDR1, CDR2, and CDR3 of SEQ ID NO: 53; the CDR1, CDR2, and CDR3 of SEQ ID NO: 57; the CDR1, CDR2, and CDR3 of SEQ ID NO: 69; the CDR1, CDR2, and CDR3 of SEQ ID NO: 73; the CDR1, CDR2, and CDR3 of SEQ ID NO: 77; the CDR1, CDR2, and CDR3 of SEQ ID NO: 81; the CDR1, CDR2, and CDR3 of SEQ ID NO: 85; the CDR1, CDR2, and CDR3 of SEQ ID NO: 89; the CDR1, CDR2, and CDR3 of SEQ ID NO: 93; the CDR1, CDR2, and CDR3 of SEQ ID NO: 97; the CDR1, CDR2, and CDR3 of SEQ ID NO: 101; the CDR1, CDR2, and CDR3 of SEQ ID NO: 105; the CDR1, CDR2, and CDR3 of SEQ ID NO: 109; the CDR1, CDR2, and CDR3 of SEQ ID NO: 117; the CDR1, CDR2, and CDR3 of SEQ ID NO: 121; the CDR1, CDR2, and CDR3 of SEQ ID NO: 125; the CDR1, CDR2, and CDR3 of SEQ ID NO: 129; the CDR1, CDR2, and CDR3 of SEQ ID NO: 133; the CDR1, CDR2, and CDR3 of SEQ ID NO: 141; the CDR1, CDR2, and CDR3 of SEQ ID NO: 145; the CDR1, CDR2, and CDR3 of SEQ ID NO: 149; the CDR1, CDR2, and CDR3 of SEQ ID NO: 153; the CDR1, CDR2, and CDR3 of SEQ ID NO: 157; the CDR1, CDR2, and CDR3 of SEQ ID NO: 161; the CDR1, CDR2, and CDR3 of SEQ ID NO: 165; the CDR1, CDR2, and CDR3 of SEQ ID NO: 169, the CDR1, CDR2, and CDR3 of SEQ ID NO: 173, the CDR1, CDR2, and CDR3 of SEQ ID NO: 177, the CDR1, CDR2, and CDR3 of SEQ ID NO: 181, or the CDR1, CDR2, and CDR3 of SEQ ID NO: 185.

In some embodiments, the variable domain of the second light chain comprises the CDR1, CDR2, and CDR3 of SEQ ID NO: 54; the CDR1, CDR2, and CDR3 of SEQ ID NO: 58; the CDR1, CDR2, and CDR3 of SEQ ID NO: 70; the CDR1, CDR2, and CDR3 of SEQ ID NO: 74; the CDR1, CDR2, and CDR3 of SEQ ID NO: 78; the CDR1, CDR2, and CDR3 of SEQ ID NO: 82; the CDR1, CDR2, and CDR3 of SEQ ID NO: 86; the CDR1, CDR2, and CDR3 of SEQ ID NO: 90; the CDR1, CDR2, and CDR3 of SEQ ID NO: 94; the CDR1, CDR2, and CDR3 of SEQ ID NO: 98; the CDR1, CDR2, and CDR3 of SEQ ID NO: 102; the CDR1, CDR2, and CDR3 of SEQ ID NO: 106; the CDR1, CDR2, and CDR3 of SEQ ID NO: 110; the CDR1, CDR2, and CDR3 of SEQ ID NO: 118; the CDR1, CDR2, and CDR3 of SEQ ID NO: 122; the CDR1, CDR2, and CDR3 of SEQ ID NO: 126; the CDR1, CDR2, and CDR3 of SEQ ID NO: 130; the CDR1, CDR2, and CDR3 of SEQ ID NO: 134; the CDR1, CDR2, and CDR3 of SEQ ID NO: 142; the CDR1, CDR2, and CDR3 of SEQ ID NO: 146; the CDR1, CDR2, and CDR3 of SEQ ID NO: 150; the CDR1, CDR2, and CDR3 of SEQ ID NO: 154; the CDR1, CDR2, and CDR3 of SEQ ID NO: 158; the CDR1, CDR2, and CDR3 of SEQ ID NO: 162, the CDR1, CDR2, and CDR3 of SEQ ID NO: 166; the CDR1, CDR2, and CDR3 of SEQ ID NO: 170, the CDR1, CDR2, and CDR3 of SEQ ID NO: 174, the CDR1, CDR2, and CDR3 of SEQ ID NO: 178, the CDR1, CDR2, and CDR3 of SEQ ID NO: 182, or the CDR1, CDR2, and CDR3 of SEQ ID NO: 186.

In some embodiments, the molecule comprises SEQ ID NO: 51, SEQ ID NO: 52, SEQ ID NO: 53, and SEQ ID NO: 54. In some embodiments, the molecule comprises SEQ ID NO: 55, SEQ ID NO: 56, SEQ ID NO: 57, and SEQ ID NO: 58. In some embodiments, the molecule comprises SEQ ID NO: 67, SEQ ID NO: 68, SEQ ID NO: 69, and SEQ ID NO: 70. In some embodiments, the molecule comprises SEQ ID NO: 71, SEQ ID NO: 72, SEQ ID NO: 73, and SEQ ID NO: 74. In some embodiments, the molecule comprises SEQ ID NO: 75, SEQ ID NO: 76, SEQ ID NO: 77, and SEQ ID NO: 78. In some embodiments, the molecule comprises SEQ ID NO: 79, SEQ ID NO: 80, SEQ ID NO: 81, and SEQ ID NO: 82. In some embodiments, the molecule comprises SEQ ID NO: 83, SEQ ID NO: 84, SEQ ID NO: 85, and SEQ ID NO: 86. In some embodiments, the molecule comprises SEQ ID NO: 87, SEQ ID NO: 88, SEQ ID NO: 89, and SEQ ID NO: 90. In some embodiments, the molecule comprises SEQ ID NO: 91, SEQ ID NO: 92, SEQ ID NO: 93, and SEQ ID NO: 94. In some embodiments, the molecule comprises SEQ ID NO: 95, SEQ ID NO: 96, SEQ ID NO: 97, and SEQ ID NO: 98. In some embodiments, the molecule comprises SEQ ID NO: 99, SEQ ID NO: 100, SEQ ID NO: 101, and SEQ ID NO: 102. In some embodiments, the molecule comprises SEQ ID NO: 103, SEQ ID NO: 104, SEQ ID NO: 105, and SEQ ID NO: 106. In some embodiments, the molecule comprises SEQ ID NO: 107, SEQ ID NO: 108, SEQ ID NO: 109, and SEQ ID NO: 110. In some embodiments, the molecule comprises SEQ ID NO: 115, SEQ ID NO: 116, SEQ ID NO: 117, and SEQ ID NO: 118. In some embodiments, the molecule comprises SEQ ID NO: 119, SEQ ID NO: 120, SEQ ID NO: 121, and SEQ ID NO: 122.

In some embodiments, the molecule comprises SEQ ID NO: 123, SEQ ID NO: 124, SEQ ID NO: 125, and SEQ ID NO: 126. In some embodiments, the molecule comprises SEQ ID NO: 127, SEQ ID NO: 128, SEQ ID NO: 129, and SEQ ID NO: 130. In some embodiments, the molecule comprises SEQ ID NO: 131, SEQ ID NO: 132, SEQ ID NO: 133, and SEQ ID NO: 133. In some embodiments, the molecule comprises SEQ ID NO: 139, SEQ ID NO: 140, SEQ ID NO: 141, and SEQ ID NO: 142. In some embodiments, the molecule comprises SEQ ID NO: 143, SEQ ID NO: 144, SEQ ID NO: 145, and SEQ ID NO: 146. In some embodiments, the molecule comprises SEQ ID NO: 147, SEQ ID NO: 148, SEQ ID NO: 149, and SEQ ID NO: 150. In some embodiments, the molecule comprises SEQ ID NO: 151, SEQ ID NO: 152, SEQ ID NO: 153, and SEQ ID NO: 154. In some embodiments, the molecule comprises SEQ ID NO: 155, SEQ ID NO: 156, SEQ ID NO: 157, and SEQ ID NO: 158. In some embodiments, the molecule comprises SEQ ID NO: 159, SEQ ID NO: 160, SEQ ID NO: 161, and SEQ ID NO: 162. In some embodiments, the molecule comprises SEQ ID NO: 163, SEQ ID NO: 164, SEQ ID NO: 165, and SEQ ID NO: 166. In some embodiments, the molecule comprises SEQ ID NO: 167, SEQ ID NO: 168, SEQ ID NO: 169, and SEQ ID NO: 170. In some embodiments, the molecule comprises SEQ ID NO: 171, SEQ ID NO: 172, SEQ ID NO: 173, and SEQ ID NO: 174. In some embodiments, the molecule comprises SEQ ID NO: 175, SEQ ID NO: 176, SEQ ID NO: 177, and SEQ ID NO: 178. In some embodiments, the molecule comprises SEQ ID NO: 179, SEQ ID NO: 180, SEQ ID NO: 181, and SEQ ID NO: 182. In some embodiments, the molecule comprises SEQ ID NO: 183, SEQ ID NO: 184, SEQ ID NO: 185, and SEQ ID NO: 186.

In some embodiments, the molecule further comprises one or more disulfide bonds. In some embodiments, the molecule further comprises one or more linker sequences. In some embodiments, the molecule is capable of neutralizing a coronavirus. In some embodiments, the coronavirus is a SARS-CoV-2 virus. In some embodiments, the SARS-CoV-2 virus strain is selected from the group consisting of WA1, B.1.1.7, B1.526, B1.351, P1, B1.352, and B.1.427.

In some embodiments, the molecule specifically recognizes a first epitope and a second epitope on a SARS-CoV-2 particle. In some embodiments, the first epitope comprises at least a portion of a receptor binding domain (RBD) on a spike protein of a SARS-CoV-2 particle. In some embodiments, the second epitope comprises at least a portion of a N-terminal domain (NTD) on a spike protein of a SARS-CoV-2 particle.

In certain aspects, the invention provides a method of treating or preventing a COVID-19 infection in a subject in need thereof, the method comprising administering to the subject a composition comprising a bispecific molecule comprising a first polypeptide chain and a second polypeptide chain, wherein: the first polypeptide chain comprises: a first light chain comprising a variable domain and a constant domain; a first heavy chain comprising a variable domain and a constant domain; the second polypeptide chain comprises: a second light chain comprising a variable domain and a constant domain; and a second heavy chain comprising a variable domain and a constant domain.

In some embodiments, the first polypeptide chain and the second polypeptide chain are covalently bonded to one another. In some embodiments, the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 51, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 55, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 67, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 71, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 75, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 79, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 83, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 87, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 91, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 95, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 99, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 103, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 107, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 115, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 119, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 123, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 127, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 131, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 139, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 143, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 147, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 151, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 155, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 159, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 163, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 167, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 171, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 175, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 179, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO:183.

In some embodiments, the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 52, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 56, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 68; a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 72, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 76, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 80, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 84, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 88, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 92, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 96, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 100, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 104, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 108, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 116, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 120, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 124, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 128, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 132, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 140, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 144, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 148, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 152, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 156, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 160, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 164, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 168, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 172, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 176, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 180, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO:184.

In some embodiments, the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 53, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 57, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 69, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 73, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 77, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 81, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 85, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 89, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 93, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 97, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 101, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 105, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 109, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 117, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 121, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 125, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 129, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 133, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 141, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 145, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 149, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 153, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 157, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 161, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 165, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 169, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 173, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 177, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 181, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 185.

In some embodiments, the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 54, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 58, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 70, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 74, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 78, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 82, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 86, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 90, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 94, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 98, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 102, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 106, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 110, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 118, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 122, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 126, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 130, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 134, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 142, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 146, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 150, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 154, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 158, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 162, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 166, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 170, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 174, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 178, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 182, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 186.

In some embodiments, the variable domain of the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 41.

In some embodiments, the variable domain of the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 42.

In some embodiments, the variable domain of the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 29, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 23, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 39, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9.

In some embodiments, variable domain of the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 30, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 24, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 40, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10.

In some embodiments, the variable domain of the first heavy chain comprises the CDR1, CDR2, and CDR3 of SEQ ID NO: 51; the CDR1, CDR2, and CDR3 of SEQ ID NO: 55; the CDR1, CDR2, and CDR3 of SEQ ID NO: 67; the CDR1, CDR2, and CDR3 of SEQ ID NO: 71; the CDR1, CDR2, and CDR3 of SEQ ID NO: 75; the CDR1, CDR2, and CDR3 of SEQ ID NO: 79; the CDR1, CDR2, and CDR3 of SEQ ID NO: 83; the CDR1, CDR2, and CDR3 of SEQ ID NO: 87; the CDR1, CDR2, and CDR3 of SEQ ID NO: 91; the CDR1, CDR2, and CDR3 of SEQ ID NO: 95; the CDR1, CDR2, and CDR3 of SEQ ID NO: 99; the CDR1, CDR2, and CDR3 of SEQ ID NO: 103; the CDR1, CDR2, and CDR3 of SEQ ID NO: 107; the CDR1, CDR2, and CDR3 of SEQ ID NO: 115; the CDR1, CDR2, and CDR3 of SEQ ID NO: 119, the CDR1, CDR2, and CDR3 of SEQ ID NO: 123, the CDR1, CDR2, and CDR3 of SEQ ID NO: 127; the CDR1, CDR2, and CDR3 of SEQ ID NO: 131; the CDR1, CDR2, and CDR3 of SEQ ID NO: 139; the CDR1, CDR2, and CDR3 of SEQ ID NO: 143; the CDR1, CDR2, and CDR3 of SEQ ID NO: 147; the CDR1, CDR2, and CDR3 of SEQ ID NO: 151; the CDR1, CDR2, and CDR3 of SEQ ID NO: 155; the CDR1, CDR2, and CDR3 of SEQ ID NO: 159; the CDR1, CDR2, and CDR3 of SEQ ID NO: 163; the CDR1, CDR2, and CDR3 of SEQ ID NO: 167, the CDR1, CDR2, and CDR3 of SEQ ID NO: 171, the CDR1, CDR2, and CDR3 of SEQ ID NO: 175, the CDR1, CDR2, and CDR3 of SEQ ID NO: 179, or the CDR1, CDR2, and CDR3 of SEQ ID NO: 183.

In some embodiments, the variable domain of the first light chain comprises the CDR1, CDR2, and CDR3 of SEQ ID NO: 52; the CDR1, CDR2, and CDR3 of SEQ ID NO: 56; the CDR1, CDR2, and CDR3 of SEQ ID NO: 68; the CDR1, CDR2, and CDR3 of SEQ ID NO: 72; the CDR1, CDR2, and CDR3 of SEQ ID NO: 76; the CDR1, CDR2, and CDR3 of SEQ ID NO: 80; the CDR1, CDR2, and CDR3 of SEQ ID NO: 84; the CDR1, CDR2, and CDR3 of SEQ ID NO: 88; the CDR1, CDR2, and CDR3 of SEQ ID NO: 92; the CDR1, CDR2, and CDR3 of SEQ ID NO: 96; the CDR1, CDR2, and CDR3 of SEQ ID NO: 100; the CDR1, CDR2, and CDR3 of SEQ ID NO: 104; the CDR1, CDR2, and CDR3 of SEQ ID NO: 108; the CDR1, CDR2, and CDR3 of SEQ ID NO: 116; the CDR1, CDR2, and CDR3 of SEQ ID NO: 120; the CDR1, CDR2, and CDR3 of SEQ ID NO: 124; the CDR1, CDR2, and CDR3 of SEQ ID NO: 128; the CDR1, CDR2, and CDR3 of SEQ ID NO: 132; the CDR1, CDR2, and CDR3 of SEQ ID NO: 140; the CDR1, CDR2, and CDR3 of SEQ ID NO: 144; the CDR1, CDR2, and CDR3 of SEQ ID NO: 148; the CDR1, CDR2, and CDR3 of SEQ ID NO: 152; the CDR1, CDR2, and CDR3 of SEQ ID NO: 156; the CDR1, CDR2, and CDR3 of SEQ ID NO: 160; the CDR1, CDR2, and CDR3 of SEQ ID NO: 164; the CDR1, CDR2, and CDR3 of SEQ ID NO: 168, the CDR1, CDR2, and CDR3 of SEQ ID NO: 172, the CDR1, CDR2, and CDR3 of SEQ ID NO: 176, the CDR1, CDR2, and CDR3 of SEQ ID NO: 180, or the CDR1, CDR2, and CDR3 of SEQ ID NO: 184.

In some embodiments, the variable domain of the second heavy chain comprises the CDR1, CDR2, and CDR3 of SEQ ID NO: 53; the CDR1, CDR2, and CDR3 of SEQ ID NO: 57; the CDR1, CDR2, and CDR3 of SEQ ID NO: 69; the CDR1, CDR2, and CDR3 of SEQ ID NO: 73; the CDR1, CDR2, and CDR3 of SEQ ID NO: 77; the CDR1, CDR2, and CDR3 of SEQ ID NO: 81; the CDR1, CDR2, and CDR3 of SEQ ID NO: 85; the CDR1, CDR2, and CDR3 of SEQ ID NO: 89; the CDR1, CDR2, and CDR3 of SEQ ID NO: 93; the CDR1, CDR2, and CDR3 of SEQ ID NO: 97; the CDR1, CDR2, and CDR3 of SEQ ID NO: 101; the CDR1, CDR2, and CDR3 of SEQ ID NO: 105; the CDR1, CDR2, and CDR3 of SEQ ID NO: 109; the CDR1, CDR2, and CDR3 of SEQ ID NO: 117; the CDR1, CDR2, and CDR3 of SEQ ID NO: 121; the CDR1, CDR2, and CDR3 of SEQ ID NO: 125; the CDR1, CDR2, and CDR3 of SEQ ID NO: 129; the CDR1, CDR2, and CDR3 of SEQ ID NO: 133; the CDR1, CDR2, and CDR3 of SEQ ID NO: 141; the CDR1, CDR2, and CDR3 of SEQ ID NO: 145; the CDR1, CDR2, and CDR3 of SEQ ID NO: 149; the CDR1, CDR2, and CDR3 of SEQ ID NO: 153; the CDR1, CDR2, and CDR3 of SEQ ID NO: 157; the CDR1, CDR2, and CDR3 of SEQ ID NO: 161; the CDR1, CDR2, and CDR3 of SEQ ID NO: 165; the CDR1, CDR2, and CDR3 of SEQ ID NO: 169, the CDR1, CDR2, and CDR3 of SEQ ID NO: 173, the CDR1, CDR2, and CDR3 of SEQ ID NO: 177, the CDR1, CDR2, and CDR3 of SEQ ID NO: 181, or the CDR1, CDR2, and CDR3 of SEQ ID NO: 185.

In some embodiments, the variable domain of the second light chain comprises the CDR1, CDR2, and CDR3 of SEQ ID NO: 54; the CDR1, CDR2, and CDR3 of SEQ ID NO: 58; the CDR1, CDR2, and CDR3 of SEQ ID NO: 70; the CDR1, CDR2, and CDR3 of SEQ ID NO: 74; the CDR1, CDR2, and CDR3 of SEQ ID NO: 78; the CDR1, CDR2, and CDR3 of SEQ ID NO: 82; the CDR1, CDR2, and CDR3 of SEQ ID NO: 86; the CDR1, CDR2, and CDR3 of SEQ ID NO: 90; the CDR1, CDR2, and CDR3 of SEQ ID NO: 94; the CDR1, CDR2, and CDR3 of SEQ ID NO: 98; the CDR1, CDR2, and CDR3 of SEQ ID NO: 102; the CDR1, CDR2, and CDR3 of SEQ ID NO: 106; the CDR1, CDR2, and CDR3 of SEQ ID NO: 110; the CDR1, CDR2, and CDR3 of SEQ ID NO: 118; the CDR1, CDR2, and CDR3 of SEQ ID NO: 122; the CDR1, CDR2, and CDR3 of SEQ ID NO: 126; the CDR1, CDR2, and CDR3 of SEQ ID NO: 130; the CDR1, CDR2, and CDR3 of SEQ ID NO: 134; the CDR1, CDR2, and CDR3 of SEQ ID NO: 142; the CDR1, CDR2, and CDR3 of SEQ ID NO: 146; the CDR1, CDR2, and CDR3 of SEQ ID NO: 150; the CDR1, CDR2, and CDR3 of SEQ ID NO: 154; the CDR1, CDR2, and CDR3 of SEQ ID NO: 158; the CDR1, CDR2, and CDR3 of SEQ ID NO: 162, the CDR1, CDR2, and CDR3 of SEQ ID NO: 166; the CDR1, CDR2, and CDR3 of SEQ ID NO: 170, the CDR1, CDR2, and CDR3 of SEQ ID NO: 174, the CDR1, CDR2, and CDR3 of SEQ ID NO: 178, the CDR1, CDR2, and CDR3 of SEQ ID NO: 182, or the CDR1, CDR2, and CDR3 of SEQ ID NO: 186.

In some embodiments, the molecule comprises SEQ ID NO: 51, SEQ ID NO: 52, SEQ ID NO: 53, and SEQ ID NO: 54. In some embodiments, the molecule comprises SEQ ID NO: 55, SEQ ID NO: 56, SEQ ID NO: 57, and SEQ ID NO: 58. In some embodiments, the molecule comprises SEQ ID NO: 67, SEQ ID NO: 68, SEQ ID NO: 69, and SEQ ID NO: 70. In some embodiments, the molecule comprises SEQ ID NO: 71, SEQ ID NO: 72, SEQ ID NO: 73, and SEQ ID NO: 74. In some embodiments, the molecule comprises SEQ ID NO: 75, SEQ ID NO: 76, SEQ ID NO: 77, and SEQ ID NO: 78. In some embodiments, the molecule comprises SEQ ID NO: 79, SEQ ID NO: 80, SEQ ID NO: 81, and SEQ ID NO: 82. In some embodiments, the molecule comprises SEQ ID NO: 83, SEQ ID NO: 84, SEQ ID NO: 85, and SEQ ID NO: 86. In some embodiments, the molecule comprises SEQ ID NO: 87, SEQ ID NO: 88, SEQ ID NO: 89, and SEQ ID NO: 90. In some embodiments, the molecule comprises SEQ ID NO: 91, SEQ ID NO: 92, SEQ ID NO: 93, and SEQ ID NO: 94. In some embodiments, the molecule comprises SEQ ID NO: 95, SEQ ID NO: 96, SEQ ID NO: 97, and SEQ ID NO: 98. In some embodiments, the molecule comprises SEQ ID NO: 99, SEQ ID NO: 100, SEQ ID NO: 101, and SEQ ID NO: 102. In some embodiments, the molecule comprises SEQ ID NO: 103, SEQ ID NO: 104, SEQ ID NO: 105, and SEQ ID NO: 106. In some embodiments, the molecule comprises SEQ ID NO: 107, SEQ ID NO: 108, SEQ ID NO: 109, and SEQ ID NO: 110. In some embodiments, the molecule comprises SEQ ID NO: 115, SEQ ID NO: 116, SEQ ID NO: 117, and SEQ ID NO: 118.

In some embodiments, the molecule comprises SEQ ID NO: 119, SEQ ID NO: 120, SEQ ID NO: 121, and SEQ ID NO: 122. In some embodiments, the molecule comprises SEQ ID NO: 123, SEQ ID NO: 124, SEQ ID NO: 125, and SEQ ID NO: 126. In some embodiments, the molecule comprises SEQ ID NO: 127, SEQ ID NO: 128, SEQ ID NO: 129, and SEQ ID NO: 130. In some embodiments, the molecule comprises SEQ ID NO: 131, SEQ ID NO: 132, SEQ ID NO: 133, and SEQ ID NO: 133. In some embodiments, the molecule comprises SEQ ID NO: 139, SEQ ID NO: 140, SEQ ID NO: 141, and SEQ ID NO: 142. In some embodiments, the molecule comprises SEQ ID NO: 143, SEQ ID NO: 144, SEQ ID NO: 145, and SEQ ID NO: 146. In some embodiments, the molecule comprises SEQ ID NO: 147, SEQ ID NO: 148, SEQ ID NO: 149, and SEQ ID NO: 150. In some embodiments, the molecule comprises SEQ ID NO: 151, SEQ ID NO: 152, SEQ ID NO: 153, and SEQ ID NO: 154. In some embodiments, the molecule comprises SEQ ID NO: 155, SEQ ID NO: 156, SEQ ID NO: 157, and SEQ ID NO: 158. In some embodiments, the molecule comprises SEQ ID NO: 159, SEQ ID NO: 160, SEQ ID NO: 161, and SEQ ID NO: 162. In some embodiments, the molecule comprises SEQ ID NO: 163, SEQ ID NO: 164, SEQ ID NO: 165, and SEQ ID NO: 166. In some embodiments, the molecule comprises SEQ ID NO: 167, SEQ ID NO: 168, SEQ ID NO: 169, and SEQ ID NO: 170. In some embodiments, the molecule comprises SEQ ID NO: 171, SEQ ID NO: 172, SEQ ID NO: 173, and SEQ ID NO: 174. In some embodiments, the molecule comprises SEQ ID NO: 175, SEQ ID NO: 176, SEQ ID NO: 177, and SEQ ID NO: 178. In some embodiments, the molecule comprises SEQ ID NO: 179, SEQ ID NO: 180, SEQ ID NO: 181, and SEQ ID NO: 182. In some embodiments, the molecule comprises SEQ ID NO: 183, SEQ ID NO: 184, SEQ ID NO: 185, and SEQ ID NO: 186.

In some embodiments, the molecule further comprises one or more disulfide bonds. In some embodiments, the molecule further comprises one or more linker sequences. In some embodiments, the molecule is capable of neutralizing a coronavirus. In some embodiments, the coronavirus is a SARS-CoV-2 virus. In some embodiments, the SARS-CoV-2 virus strain is selected from the group consisting of WA1, B.1.1.7, B1.526, B1.351, P1, B1.352, and B.1.427. In some embodiments, the molecule specifically recognizes a first epitope and a second epitope on a SARS-CoV-2 particle. In some embodiments, the first epitope comprises at least a portion of a receptor binding domain (RBD) on a spike protein of a SARS-CoV-2 particle. In some embodiments, the second epitope comprises at least a portion of a N-terminal domain (NTD) on a spike protein of a SARS-CoV-2 particle. In some embodiments, the composition is administered in combination with a second therapeutic agent.

In certain aspects, the invention provides a synthesized monoclonal antibody, or a functional fragment thereof, comprising a heavy chain having a variable domain and a constant domain and a light chain having a variable domain and a constant domain, wherein the antibody binds an antigen on a coronavirus particle.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 41. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 23. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 42. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 2.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 24. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 41 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 42. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 2. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 23 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 24. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28.

In some embodiments, the heavy chain variable domain comprises SEQ ID NO: 5 and the light chain variable domain comprises SEQ ID NO: 6. In some embodiments, the heavy chain variable domain comprises SEQ ID NO: 41 and the light chain variable domain comprises SEQ ID NO: 42. In some embodiments, the heavy chain variable domain comprises SEQ ID NO: 35 and the light chain variable domain comprises SEQ ID NO: 36. In some embodiments, the heavy chain variable domain comprises SEQ ID NO: 1 and the light chain variable domain comprises SEQ ID NO: 2. In some embodiments, the heavy chain variable domain comprises SEQ ID NO: 23 and the light chain variable domain comprises SEQ ID NO: 24. In some embodiments, the heavy chain variable domain comprises SEQ ID NO: 13 and the light chain variable domain comprises SEQ ID NO: 14. In some embodiments, the heavy chain variable domain comprises SEQ ID NO: 27 and the light chain variable domain comprises SEQ ID NO: 28. In some embodiments, the heavy chain variable domain comprises the CDR1, CDR2, and CDR3 regions of SEQ ID NO: 5. In some embodiments, the heavy chain variable domain comprises the CDR1, CDR2, and CDR3 regions of SEQ ID NO: 41. In some embodiments, the heavy chain variable domain comprises the CDR1, CDR2, and CDR3 regions of SEQ ID NO: 1.

In some embodiments, the heavy chain variable domain comprises the CDR1, CDR2, and CDR3 regions of SEQ ID NO: 23. In some embodiments, the heavy chain variable domain comprises the CDR1, CDR2, and CDR3 regions of SEQ ID NO: 13. In some embodiments, the heavy chain variable domain comprises the CDR1, CDR2, and CDR3 regions of SEQ ID NO: 27.

In some embodiments, the light chain variable domain comprises the CDR1, CDR2, and CDR3 regions of SEQ ID NO: 6. In some embodiments, the light chain variable domain comprises the CDR1, CDR2, and CDR3 regions of SEQ ID NO: 42. In some embodiments, the light chain variable domain comprises the CDR1, CDR2, and CDR3 regions of SEQ ID NO: 36. In some embodiments, the light chain variable domain comprises the CDR1, CDR2, and CDR3 regions of SEQ ID NO: 2. In some embodiments, the light chain variable domain comprises the CDR1, CDR2, and CDR3 regions of SEQ ID NO: 24. In some embodiments, the light chain variable domain comprises the CDR1, CDR2, and CDR3 regions of SEQ ID NO: 14. In some embodiments, the light chain variable domain comprises the CDR1, CDR2, and CDR3 regions of SEQ ID NO: 28. In some embodiments, the heavy chain variable domain comprises the CDR1, CDR2, and CDR3 regions of SEQ ID NO: 5 and the light chain variable domain comprises the CDR1, CDR2, and CDR3 regions of SEQ ID NO: 6. In some embodiments, the heavy chain variable domain comprises the CDR1, CDR2, and CDR3 regions of SEQ ID NO: 41 and the light chain variable domain comprises the CDR1, CDR2, and CDR3 regions of SEQ ID NO: 42. In some embodiments, the heavy chain variable domain comprises the CDR1, CDR2, and CDR3 regions of SEQ ID NO: 35 and the light chain variable domain comprises the CDR1, CDR2, and CDR3 regions of SEQ ID NO: 36. In some embodiments, the heavy chain variable domain comprises the CDR1, CDR2, and CDR3 regions of SEQ ID NO: 1 and the light chain variable domain comprises the CDR1, CDR2, and CDR3 regions of SEQ ID NO: 2.

In some embodiments, the heavy chain variable domain comprises the CDR1, CDR2, and CDR3 regions of SEQ ID NO: 23 and the light chain variable domain comprises the CDR1, CDR2, and CDR3 regions of SEQ ID NO: 24. In some embodiments, the heavy chain variable domain comprises the CDR1, CDR2, and CDR3 regions of SEQ ID NO: 13 and the light chain variable domain comprises the CDR1, CDR2, and CDR3 regions of SEQ ID NO: 14. In some embodiments, the heavy chain variable domain comprises the CDR1, CDR2, and CDR3 regions of SEQ ID NO: 27 and the light chain variable domain comprises the CDR1, CDR2, and CDR3 regions of SEQ ID NO: 28.

In some embodiments, the antibody neutralizes a coronavirus. In some embodiments, the coronavirus is a SARS-CoV-2 virus. In some embodiments, the coronavirus is a SARS-CoV-2 variant strain. In some embodiments, the variant strain is selected from the group consisting of WA1, B.1.1.7, B1.526, B1.351, P1, B1.352, and B.1.427. In some embodiments, the antibody specifically binds an epitope on the surface of the coronavirus. In some embodiments, the epitope comprises at least a portion of a coronavirus spike protein. In some embodiments, the at least a portion of a coronavirus spike protein comprises a receptor binding domain (RBD) of a spike protein. In some embodiments, the at least a portion of a coronavirus spike protein comprises a N-Terminal domain (NTD) of a spike protein. In some embodiments, the coronavirus is a SARS-CoV-2 coronavirus.

In certain aspects, the invention provides a method of treating or preventing a COVID-19 infection in a subject in need thereof, the method comprising administering to the subject a therapeutically effective amount of a synthesized monoclonal antibody, or a functional fragment thereof, comprising a heavy chain having a variable domain and a constant domain and a light chain having a variable domain and a constant domain, wherein the antibody binds an antigen on a coronavirus particle.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 41. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 23. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 42. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 2. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 24. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 41 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 42. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 2. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 23 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 24.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28.

In some embodiments, the heavy chain variable domain comprises SEQ ID NO: 5 and the light chain variable domain comprises SEQ ID NO: 6. In some embodiments, the heavy chain variable domain comprises SEQ ID NO: 41 and the light chain variable domain comprises SEQ ID NO: 42. In some embodiments, the heavy chain variable domain comprises SEQ ID NO: 35 and the light chain variable domain comprises SEQ ID NO: 36. In some embodiments, the heavy chain variable domain comprises SEQ ID NO: 1 and the light chain variable domain comprises SEQ ID NO: 2. In some embodiments, the heavy chain variable domain comprises SEQ ID NO: 23 and the light chain variable domain comprises SEQ ID NO: 24. In some embodiments, the heavy chain variable domain comprises SEQ ID NO: 13 and the light chain variable domain comprises SEQ ID NO: 14.

In some embodiments, the heavy chain variable domain comprises SEQ ID NO: 27 and the light chain variable domain comprises SEQ ID NO: 28. In some embodiments, the heavy chain variable domain comprises the CDR1, CDR2, and CDR3 regions of SEQ ID NO: 5. In some embodiments, the heavy chain variable domain comprises the CDR1, CDR2, and CDR3 regions of SEQ ID NO: 41. In some embodiments, the heavy chain variable domain comprises the CDR1, CDR2, and CDR3 regions of SEQ ID NO: 1. In some embodiments, the heavy chain variable domain comprises the CDR1, CDR2, and CDR3 regions of SEQ ID NO: 23. In some embodiments, the heavy chain variable domain comprises the CDR1, CDR2, and CDR3 regions of SEQ ID NO: 13. In some embodiments, the heavy chain variable domain comprises the CDR1, CDR2, and CDR3 regions of SEQ ID NO: 27. In some embodiments, the light chain variable domain comprises the CDR1, CDR2, and CDR3 regions of SEQ ID NO: 6. In some embodiments, the light chain variable domain comprises the CDR1, CDR2, and CDR3 regions of SEQ ID NO: 42. In some embodiments, the light chain variable domain comprises the CDR1, CDR2, and CDR3 regions of SEQ ID NO: 36. In some embodiments, the light chain variable domain comprises the CDR1, CDR2, and CDR3 regions of SEQ ID NO: 2. In some embodiments, the light chain variable domain comprises the CDR1, CDR2, and CDR3 regions of SEQ ID NO: 24.

In some embodiments, the light chain variable domain comprises the CDR1, CDR2, and CDR3 regions of SEQ ID NO: 14. In some embodiments, the light chain variable domain comprises the CDR1, CDR2, and CDR3 regions of SEQ ID NO: 28. In some embodiments, the heavy chain variable domain comprises the CDR1, CDR2, and CDR3 regions of SEQ ID NO: 5 and the light chain variable domain comprises the CDR1, CDR2, and CDR3 regions of SEQ ID NO: 6. In some embodiments, the heavy chain variable domain comprises the CDR1, CDR2, and CDR3 regions of SEQ ID NO: 41 and the light chain variable domain comprises the CDR1, CDR2, and CDR3 regions of SEQ ID NO: 42. In some embodiments, the heavy chain variable domain comprises the CDR1, CDR2, and CDR3 regions of SEQ ID NO: 35 and the light chain variable domain comprises the CDR1, CDR2, and CDR3 regions of SEQ ID NO: 36. In some embodiments, the heavy chain variable domain comprises the CDR1, CDR2, and CDR3 regions of SEQ ID NO: 1 and the light chain variable domain comprises the CDR1, CDR2, and CDR3 regions of SEQ ID NO: 2. In some embodiments, the heavy chain variable domain comprises the CDR1, CDR2, and CDR3 regions of SEQ ID NO: 23 and the light chain variable domain comprises the CDR1, CDR2, and CDR3 regions of SEQ ID NO: 24. In some embodiments, the heavy chain variable domain comprises the CDR1, CDR2, and CDR3 regions of SEQ ID NO: 13 and the light chain variable domain comprises the CDR1, CDR2, and CDR3 regions of SEQ ID NO: 14. In some embodiments, the heavy chain variable domain comprises the CDR1, CDR2, and CDR3 regions of SEQ ID NO: 27 and the light chain variable domain comprises the CDR1, CDR2, and CDR3 regions of SEQ ID NO: 28.

In some embodiments, the antibody neutralizes a coronavirus. In some embodiments, the coronavirus is a SARS-CoV-2 virus. In some embodiments, the coronavirus is a SARS-CoV-2 variant strain. In some embodiments, the variant strain is selected from the group consisting of WA1, B.1.1.7, B1.526, B1.351, P1, B1.352, and B.1.427. In some embodiments, the antibody specifically binds an epitope on the surface of the coronavirus. In some embodiments, the epitope comprises at least a portion of a coronavirus spike protein. In some embodiments, the at least a portion of a coronavirus spike protein comprises a receptor binding domain (RBD) of a spike protein. In some embodiments, the at least a portion of a coronavirus spike protein comprises a N-Terminal domain (NTD) of a spike protein. In some embodiments, the coronavirus is a SARS-CoV-2 coronavirus. In some embodiments, the antibody is administered in combination with a second pharmaceutical agent.

In certain aspects, the invention provides a bispecific molecule comprising a first polypeptide chain and a second polypeptide chain, wherein: the first polypeptide chain comprises: a first light chain comprising a variable domain and a constant domain; a first heavy chain comprising a variable domain and a constant domain; the second polypeptide chain comprises: a second light chain comprising a variable domain and a constant domain; and a second heavy chain comprising a variable domain and a constant domain. In some embodiments, the first polypeptide chain and the second polypeptide chain are covalently bonded to one another.

In some embodiments, the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 47, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 111, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 135, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 187, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 211. In some embodiments, the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 48, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 112, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 136, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 188, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 212.

In some embodiments, the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 49, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 113, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 137, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 189, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 213. In some embodiments, the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 50, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 114, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 138, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 190, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 214. In some embodiments, the variable domain of the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13.

In some embodiments, the variable domain of the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14. In some embodiments, the variable domain of the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 45, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43. In some embodiments, the variable domain of the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 46, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44.

In some embodiments, the variable domain of the first heavy chain comprises CDR1, CDR2, and CDR3 of SEQ ID NO: 47; CDR1, CDR2, and CDR3 of SEQ ID NO: 111; CDR1, CDR2, and CDR3 of SEQ ID NO: 135; CDR1, CDR2, and CDR3 of SEQ ID NO: 187; or CDR1, CDR2, and CDR3 of SEQ ID NO: 211. In some embodiments, the variable domain of the first light chain comprises CDR1, CDR2, and CDR3 of SEQ ID NO: 48, CDR1, CDR2, and CDR3 of SEQ ID NO: 112; CDR1, CDR2, and CDR3 of SEQ ID NO: 136; CDR1, CDR2, and CDR3 of SEQ ID NO: 188; or CDR1, CDR2, and CDR3 of SEQ ID NO: 212. In some embodiments, the variable domain of the second heavy chain comprises CDR1, CDR2, and CDR3 of SEQ ID NO: 49, CDR1, CDR2, and CDR3 of SEQ ID NO: 113; CDR1, CDR2, and CDR3 of SEQ ID NO: 137; CDR1, CDR2, and CDR3 of SEQ ID NO: 189; or CDR1, CDR2, and CDR3 of SEQ ID NO: 213. In some embodiments, the variable domain of the second light chain comprises CDR1, CDR2, and CDR3 SEQ ID NO: 50, CDR1, CDR2, and CDR3 of SEQ ID NO: 114; CDR1, CDR2, and CDR3 of SEQ ID NO: 138; CDR1, CDR2, and CDR3 of SEQ ID NO: 190; or CDR1, CDR2, and CDR3 of SEQ ID NO: 214.

In some embodiments, the molecule comprises SEQ ID NO: 47, SEQ ID NO: 48, SEQ ID NO: 49, and SEQ ID NO: 50. In some embodiments, the molecule comprises SEQ ID NO:111, SEQ ID NO: 112, SEQ ID NO: 113, and SEQ ID NO: 114. In some embodiments, the molecule comprises SEQ ID NO: 135, SEQ ID NO: 135, SEQ ID NO: 137, and SEQ ID NO: 138. In some embodiments, the molecule comprises SEQ ID NO: 187, SEQ ID NO: 188, SEQ ID NO: 189, and SEQ ID NO: 190. In some embodiments, the molecule comprises SEQ ID NO: 211, SEQ ID NO: 212, SEQ ID NO: 213, and SEQ ID NO: 214.

In some embodiments, the molecule further comprises one or more disulfide bonds. In some embodiments, the molecule further comprises one or more linker sequences. In some embodiments, the molecule is capable of neutralizing a coronavirus. In some embodiments, the coronavirus is a SARS-CoV-2 virus. In some embodiments, the SARS-CoV-2 virus strain is selected from the group consisting of WA1, B.1.1.7, B1.526, B1.351, P1, B1.352, and B.1.427. In some embodiments, the molecule specifically recognizes a first epitope and a second epitope on a SARS-CoV-2 particle. In some embodiments, the first epitope comprises at least a portion of a receptor binding domain (RBD) on a spike protein of a SARS-CoV-2 particle. In some embodiments, the second epitope comprises at least a portion of a N-terminal domain (NTD) on a spike protein of a SARS-CoV-2 particle.

In certain aspects, the invention provides a method of treating or preventing a COVID-19 infection in a subject in need thereof, the method comprising administering to the subject a composition comprising a bispecific molecule comprising a first polypeptide chain and a second polypeptide chain, wherein: the first polypeptide chain comprises: a first light chain comprising a variable domain and a constant domain; a first heavy chain comprising a variable domain and a constant domain; the second polypeptide chain comprises: a second light chain comprising a variable domain and a constant domain; and a second heavy chain comprising a variable domain and a constant domain. In some embodiments, the first polypeptide chain and the second polypeptide chain are covalently bonded to one another.

In some embodiments, the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 47, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 111, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 135, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 187, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 211.

In some embodiments, the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 48, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 112, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 136, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 188, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 212.

In some embodiments, the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 49, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 113, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 137, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 189, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 213.

In some embodiments, the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 50, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 114, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 138, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 190, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 214.

In some embodiments, the variable domain of the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13. In some embodiments, the variable domain of the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14. In some embodiments, the variable domain of the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 45, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43.

In some embodiments, the variable domain of the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 46, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44.

In some embodiments, the variable domain of the first heavy chain comprises CDR1, CDR2, and CDR3 of SEQ ID NO: 47; CDR1, CDR2, and CDR3 of SEQ ID NO: 111; CDR1, CDR2, and CDR3 of SEQ ID NO: 135; CDR1, CDR2, and CDR3 of SEQ ID NO: 187; or CDR1, CDR2, and CDR3 of SEQ ID NO: 211. In some embodiments, the variable domain of the first light chain comprises CDR1, CDR2, and CDR3 of SEQ ID NO: 48, CDR1, CDR2, and CDR3 of SEQ ID NO: 112; CDR1, CDR2, and CDR3 of SEQ ID NO: 136; CDR1, CDR2, and CDR3 of SEQ ID NO: 188; or CDR1, CDR2, and CDR3 of SEQ ID NO: 212. In some embodiments, the variable domain of the second heavy chain comprises CDR1, CDR2, and CDR3 of SEQ ID NO: 49, CDR1, CDR2, and CDR3 of SEQ ID NO: 113; CDR1, CDR2, and CDR3 of SEQ ID NO: 137; CDR1, CDR2, and CDR3 of SEQ ID NO: 189; or CDR1, CDR2, and CDR3 of SEQ ID NO: 213. In some embodiments, the variable domain of the second light chain comprises CDR1, CDR2, and CDR3 SEQ ID NO: 50, CDR1, CDR2, and CDR3 of SEQ ID NO: 114; CDR1, CDR2, and CDR3 of SEQ ID NO: 138; CDR1, CDR2, and CDR3 of SEQ ID NO: 190; or CDR1, CDR2, and CDR3 of SEQ ID NO: 214. In some embodiments, the molecule comprises SEQ ID NO: 47, SEQ ID NO: 48, SEQ ID NO: 49, and SEQ ID NO: 50. In some embodiments, the molecule comprises SEQ ID NO:111, SEQ ID NO: 112, SEQ ID NO: 113, and SEQ ID NO: 114. In some embodiments, the molecule comprises SEQ ID NO: 135, SEQ ID NO: 135, SEQ ID NO: 137, and SEQ ID NO: 138. In some embodiments, the molecule comprises SEQ ID NO: 187, SEQ ID NO: 188, SEQ ID NO: 189, and SEQ ID NO: 190. In some embodiments, the molecule comprises SEQ ID NO: 211, SEQ ID NO: 212, SEQ ID NO: 213, and SEQ ID NO: 214.

In some embodiments, the molecule further comprises one or more disulfide bonds. In some embodiments, the molecule further comprises one or more linker sequences. In some embodiments, the molecule is capable of neutralizing a coronavirus. In some embodiments, the coronavirus is a SARS-CoV-2 virus. In some embodiments, the SARS-CoV-2 virus strain is selected from the group consisting of WA1, B.1.1.7, B1.526, B1.351, P1, B1.352, and B.1.427.

In some embodiments, the molecule specifically recognizes a first epitope and a second epitope on a SARS-CoV-2 particle. In some embodiments, the first epitope comprises at least a portion of a receptor binding domain (RBD) on a spike protein of a SARS-CoV-2 particle. In some embodiments, the second epitope comprises at least a portion of a N-terminal domain (NTD) on a spike protein of a SARS-CoV-2 particle. In some embodiments, the composition is administered in combination with a second therapeutic agent.

### BRIEF DESCRIPTION OF FIGURES

The patent or application file contains at least one drawing originally in color. To conform to the requirements for PCT patent applications, many of the figures presented herein are black and white representations of images originally created in color.

FIGS. 1A-C show images of coronavirus. FIG. 1A shows a SARS-CoV-2 viral particle schematic diagram. FIG. 1B shows a spike protein trimer structure. FIG. 1C shows an electronic micrograph of coronavirus particles. The arrow denotes the viral spike protein on the viral membrane.

FIGS. 2A-G show individual antibody responses to SARS-CoV-2 virus. FIGS. 2A-F shows ELISA binding assays in which various COVID-19 patient plasma samples (indicated by different colored lines) were tested for their ability to bind to SARS-CoV-2 nucleocapsid antigen (FIGS. 2A and D), SARS-CoV-2 envelope trimer antigen (FIGS. 2B and E), and SARS-CoV envelope trimer antigen (FIGS. 2C and F). FIGS. 2A-C show responses for patients with severe disease. FIGS. 2D-F show responses for patients with non-severe disease. FIG. 2G shows that a higher SARS-CoV-2 antibody responses are present in plasma samples of patients with severe COVID-19 as compared to patients with non-severe COVID-19.

FIGS. 3A-J show antibody responses that neutralize SARS-CoV-2 potently.

FIGS. 3A-F show individual neutralization assays for patients with severe disease (FIGS. 3A-C) and patients with non-severe disease (FIGS. 3D-F). FIGS. 3A and D show neutralization of SARS-CoV-2 pseudovirus. FIGS. 3B and E show neutralization of SARS-CoV pseudovirus. FIGS. 3C and F show neutralization of SARS-CoV-2 live virus. FIG. 3G shows a plot of the infective dose (ID₅₀), or the estimated number of virus particles required to produce a 50% infection, obtained from FIGS. 3A-F. FIGS. 3H-J show correlation of the IC₅₀ values obtained by pseudovirus neutralization assays with live virus neutralization IC50 (FIG. 311), half-maximal plasma dilution (FIG. 3I), and 1:400 OD₄₅₀ (FIG. 3J).

FIGS. 4A-C show schematics for identifying potent neutralizing antibodies against COVID-19. FIG. 4A shows a schematic of producing potent neutralizing antibodies.

FIG. 4B shows memory B cells sorted from a healthy individual and a COVID-19 patient.

FIG. 4C shows a table of seven patient donors and the number of antibodies isolated from each donor.

FIGS. 5A-D show binding and neutralization assays of mAbs against the SARS-CoV-2 virus. FIG. 5A shows a binding assay of synthesized antibodies with the SARS-CoV-2 spike protein trimer. FIG. 5B shows a binding assay of synthesized antibodies with the SARS-CoV-2 receptor binding domain. FIG. 5C shows neutralization assay of synthesized antibodies with SARS-CoV-2 pseudovirus. FIG. 5D shows neutralization assay of synthesized antibodies with SARS-CoV-2 live virus.

FIGS. 6A-F show epitope mapping of SARS-CoV-2 specific monoclonal antibodies by competition ELISA assay. FIGS. 6A-C show individual synthesized mAbs binding affinity to the receptor-binding domain (RBD), with antibody numbers 2-4, 2-15, and 2-38 specifically binding to the RBD. FIGS. 6D-F show individual synthesized mAbs binding affinity to the receptor-binding domain (RBD), with antibody numbers 2-51, 4-8, and 4-32 showing no binding affinity to the RBD.

FIGS. 7A-E show surface plasmon resonance (SPR) assay for mAb binding to the spike protein trimers. Each panel represents the binding affinity and binding kinetics of the indicated monoclonal antibody (i.e., 2-4, 2-15, 2-38, etc.) to the SARS-CoV-2 envelope timer. Colored lines indicated different dilutions of the respective antibody tested. FIG. 7A shows mAb 2-4. FIG. 7B shows mAb 2-15. FIG. 7C shows mAb 2-38. FIG. 7D shows mAb 2-51. FIG. 7E shows mAb 4-8.

FIGS. 8A-D show initial screenings of mAbs that bind and neutralize the SARS-CoV-2 virus. FIG. 8A shows mAbs binding to spike trimer protein. FIG. 8B shows mAbs binding to the receptor-binding domain (RBD). FIG. 8C shows IC₅₀ values for pseudotyped virus infection. FIG. 8D shows percent inhibition values for live virus infection.

FIG. 9 shows a summary of the SARS-CoV-2-specific mAb screening. Number of most potent neutralizing mAbs: 17.

FIGS. 10A-I show binding of potent neutralizing mAbs to spike protein trimer, RBD and N-terminal domain (NTD). FIGS. 10A, D, and G show binding to spike protein trimer. FIGS. 10B, E, and H show binding to the RBD. FIGS. 10C, F, and I show binding to the NTD. FIGS. 10A-C show binding of mAbs, which potently bind the RBD. FIGS. 10D-F show binding of mAbs, which potently bind the NTD. FIGS. 10G-I show binding of mAb, which bind epitopes other than the RBD or the NTD.

FIGS. 11A-F show SARS-CoV-2 neutralization activity of select mAbs. FIG. 11A shows neutralization activity of RBD-targeting mAbs neutralizing a pseudovirus infection. FIG. 11B shows neutralization activity of NTD-targeting mAbs neutralizing a pseudovirus infection. FIG. 11C shows neutralization activity of mAbs, which target epitopes other than the RBD or the NTD, neutralizing a pseudovirus infection. FIG. 11D shows neutralization activity of RBD-targeting mAbs neutralizing a live virus infection. FIG. 11E shows neutralization activity of NTD-targeting mAbs neutralizing a live virus infection. FIG. 11F shows neutralization activity of mAbs, which target epitopes other than the RBD or the NTD, neutralizing a live virus infection.

FIGS. 12A-B show a CryoEM structure of an RBD-targeting monoclonal antibody. FIG. 12A shows a side view of the mAb. FIG. 12B shows a top view of the mAb.

FIGS. 13A-B show a CryoEM structure of an NTD-targeting monoclonal antibody. FIG. 13A shows a side view of the mAb. FIG. 13B shows a top view of the mAb.

FIG. 14 shows three SARS-CoV-2 neutralizing epitope clusters identified on the spike protein trimer.

FIGS. 15A-E show isolation of SARS-CoV-2 mAbs from infected patients with severe disease. FIG. 15A shows plasma neutralization profile of 40 patients against SARS-CoV-2 pseudovirus (highlighted are 5 top neutralizers chosen). All 252 transfection supernatants were screened for binding to S trimer and RBD, as well as for neutralization against SARS-CoV-2 pseudovirus and live virus. For pseudovirus neutralization, the 50% inhibitory dilutions (IC50) of each supernatant were plotted. For live virus, semi-quantitative representation of the inhibition at a dilution of 1:50, with neutralization levels ranging from (−) for none to (+++) for complete neutralization, was plotted. Potent antibodies later identified are marked by vertical lines and labelled at the bottom. The antibodies from each patient are formatted or colored as in FIG. 15A. FIG. 15B shows s trimer binding. FIG. 15C shows RBD binding. FIG. 15D shows pseudovirus neutralization. FIG. 15E shows live virus neutralization.

FIGS. 16A-0 show characterization of SARS-CoV-2 potent neutralizing mAbs. FIGS. 16A, D, and G show binding profiles of 19 purified potent neutralizing mAbs against SARS-CoV-2 S trimer (left). FIGS. 16B, E, and H show binding profiles of 19 purified potent neutralizing mAbs against SARS-CoV-2 RBD (middle). FIGS. 16C, F, and I show binding profiles of 19 purified potent neutralizing mAbs against SARS-CoV-2 NTD (right). Note that mAb 2-30 bound multiple proteins at high concentrations. FIG. 16J shows pseudovirus neutralization against SARS-CoV-2 RBD. FIG. 16K shows pseudovirus neutralization against SARS-CoV-2 NTD. FIG. 16L shows pseudovirus neutralization against other epitopes on the SARS-CoV-2 virion. FIG. 16M shows live virus neutralization against SARS-CoV-2 RBD. FIG. 16N shows live virus neutralization against SARS-CoV-2 NTD. FIG. 16O shows live virus neutralization against other epitopes on the SARS-CoV-2 virion. The neutralization profiles are for the 19 purified mAbs. Single replicate of the binding experiment and triplicates of neutralization are presented as mean±SEM.

FIGS. 17A-D show epitope mapping of select neutralizing and non-neutralizing mAbs. Competition results of non-RBD binders (FIG. 17A) and RBD binders (FIG. 17B) in blocking ACE2 or biotinylated mAb binding to the S trimer. In addition, the ability to bind NTD and RBD of each mAb is shown. The numbers in each box show the area under each competition curve (AUC) as tested by ELISA. +/− indicates binding/no binding of the mAb to the protein. FIG. 17C shows a Venn diagram interpretation of results from FIG. 17B. FIG. 17D shows a Venn diagram interpretation of results from FIG. 17B.

FIGS. 18A-D show Cryo-EM reconstructions of Fab-spike complexes and visualization of neutralizing epitopes on the spike surface. FIG. 18A shows a Cryo-EM 3D reconstruction of antibody 2-4 in complex with S trimer at 3.2 Å overall resolution. Density is colored according to spike domain with RBD in green, NTD in orange, with other regions colored grey. FIG. 18B shows a Cryo-EM reconstruction of antibody 4-8 in complex with S trimer (ribbon diagram, colored as in a) at 3.9 Å overall resolution, with RBDs in the “all-down” configuration. The resolution of antibody density is limited by molecular motion. Although the binding of Fab to NTD and antibody position are clear, the identities of heavy and light chain are uncertain. FIG. 18C shows a Cryo-EM reconstruction of the antibody 2-43 in complex with S trimer at 7.8 Å resolution reveals a quaternary epitope involving RBD from one subunit and NTD from the next. FIG. 18D shows mapping of the Venn diagrams from FIG. 17B onto the surface of the viral spike.

FIG. 19 shows Patient information. Abbreviation: ARDS, acute respiratory distress syndrome; MV, mechanical ventilation; hsCRP, high sensitivity C-reactive protein, ULN>10 mg/L; ESR, erythrocyte sedimentation rate, ULN=20 mm/hr; Interleukin 6, ULN=5 pg/mL; Ferritin, ULN=150 ng/mL; D-dimer quantitative ULN=0.8 μg/mL FEU.

FIG. 20 shows summary of mAb screening.

FIG. 21 shows Cryo-EM data collection, refinement, and validation statistics.

FIGS. 22A-C show SARS-CoV-2 S trimer-specific antibody isolation strategy. FIG. 22A shows a schema for isolating of S trimer-specific mAbs from memory B cells in the blood of infected patients. FIG. 22B shows sorting results on the isolation of S trimer-specific memory B cells using flow cytometry. FIG. 22C shows magnified representation of the panel of S trimer-positive memory B cells for each patient. Inset numbers indicate the absolute number and the percentage of S trimer-specific memory B cells isolated from each case.

FIGS. 23A-G show Genetic features of SARS-CoV-2-specific antibody repertoire. FIG. 23A shows that most of the 121 trimer S-specific antibodies are of IgG isotype. FIG. 23B shows the heavy chains The kappa (FIG. 23C) and lambda (FIG. 23D) light chains are comparably used. Compared to IgG repertoires of healthy human donors, IGHV3-30 and IGKV3-20 genes are over-represented in heavy and light chain repertoires, respectively (132-test, p<0.05). FIG. 23E shows that the usage of IGHJ6 gene was significantly higher in antigen-specific antibodies (132-test, p<0.05). FIG. 23F shows that the CDRH3 length of antigen-specific antibodies is significantly longer than in healthy donors (Kolmogorov—Smirnov test, p=0.014). FIG. 23G shows that for both heavy and light chains, the V region nucleotide somatic hypermutation levels are significantly lower than in antibodies of healthy donors (Kolmogorov—Smirnov test, p<0.001).

FIG. 24 shows The best-fit pseudovirus neutralization curves for 130 samples that were positive in at least one of the screens shown in FIGS. 15B-E. The 18 transfection supernatants that showed evidently better potency are highlighted in colors, while others with non-neutralizing or weakly neutralizing activities are shown in grey. One additional supernatant (Patient 1) that was initially missed in the pseudovirus screen but later found to be a potent neutralizing mAb (1-87) is also highlighted.

FIG. 25 shows The pseudovirus neutralization profiles for 12 purified mAbs that strongly bound the S trimer but with weak or no virus-neutralizing activities. The four mAbs with weak neutralizing activities against SARS-CoV-2 pseudovirus are shown in sold lines, and the remaining 8 non-neutralizing mAbs are shown in dashed lines.

FIGS. 26A-C shows monoclonal Ab 2-43 bound to S trimer expressed on Expi293 cell surface can be competed out by mAbs directed to RBD but only minimally by mAbs to the NTD region. FIG. 26A shows results for 2-43 and proteins. FIG. 26B shows results for NTD-directed mAbs. FIG. 26C shows results for RBD-directed mAbs.

FIGS. 27A-F show Cryo-EM data processing for antibody 2-4 in complex with the S trimer. FIG. 27A shows a representative micrograph and CTF of the micrograph. FIG. 27B shows a representative 2D class averages. FIG. 27C shows a resolution of the consensus map with C3 symmetry as calculated by 3DFSC. FIG. 27D shows the local resolution of the full map as calculated by cryoSPARC at an FSC cutoff of 0.5. Representative density of the Fab 2-4 and RBD interface, showing CRR H3, L3, and L3 (FIG. 27E), along with CDR H2 and the N-linked glycosylation at ASN58 (FIG. 27F).

FIGS. 28A-E show Fab 2-4 binding. FIG. 28A shows Fab 2-4 binding interface with RBD. FIG. 28B shows positions of antibodies 2-4, S309⁸, and BD-23⁹ on the trimeric CoV-2 spike. FIG. 28C shows somatic hypermutations found only in the antibody 2-4 heavy chain, shown in brown. The mutation A60T creates an N×T sequence leading to N58 glycosylation. FIG. 28D shows antibody 2-4 in complex with S trimer. CDR loops are indicated in colors, 751 and side chains are shown for interacting residues. FIG. 28E shows antibody BD-23⁹ in complex with S trimer.

FIGS. 29A-F show Cryo-EM data processing for antibody 4-8 in complex with the S trimer. FIG. 29A shows a representative micrograph and CTF of the micrograph. FIG. 29B shows a representative 2D class averages. FIG. 29C shows resolution of the spike in the RBD down conformation in complex with Fab 4-8. FIG. 29D shows resolution of the spike in the RBD up conformation in complex with Fab 4-8. FIG. 29E shows local resolution of the spike in the RBD down conformation in complex with Fab 4-8 at an FSC cutoff of 0.5. Two thresholds are shown. FIG. 29F shows local resolution of the spike in the RBD up conformation in complex with Fab 4-8 at an FSC cutoff of 0.5. Two thresholds are shown.

FIG. 30 shows 3D reconstructions of NTD-targeting neutralizing antibody 4-8 in complex with the SARS-CoV-2 spike trimer with the 1-RBD-up conformation.

FIGS. 31A-D show Cryo-EM data processing for antibody 2-43 in complex with the S trimer. FIG. 31A shows a representative micrograph and CTF of the micrograph. FIG. 31B shows representative 2D class averages. FIG. 31C shows resolution of Fab 2-43 in complex with S trimer. FIG. 31D shows the local resolution of the full map as calculated by cryoSPARC at an FSC cutoff of 0.5.

FIGS. 32A-B show monoclonal antibody 2-15 mut. FIG. 32A shows virus neutralization with the 2-15 mut antibody. FIG. 32B shows potency of the 2-15 mut antibody.

FIGS. 33A-B show select mutants of the 4-8 monoclonal antibody. FIG. 33A shows neutralization with antibodies 4-8 (39/51) and 4-8(39/51/57). FIG. 33B shows potency of antibodies 4-8 (39/51) and 4-8(39/51/57).

FIG. 34 shows IC₅₀ and IC₉₀ values of selected monoclonal antibody mutants.

FIG. 35 shows that antibody 2-36 is a potent neutralizer against SARS-CoV pseudovirus.

FIGS. 36A-B show ELISA binding of antibody 2-36 to SARS-CoV-2 Spike trimer (FIG. 36A) and SARS-CoV Spike trimer (FIG. 36B). Antibody CR3022, an antibody previously reported to have cross-activity, served as a control.

FIGS. 37A-D show the binding affinities of antibody 2-36 and antibody 2-4 to SARS-CoV-2 Spike trimer and to SARS-CoV Spike trimer, as measured by SPR. Antibody 2-36 was confirmed to binds to both SARS-CoV-2 (FIG. 37A) and SARS-CoV (FIG. 37B) spikes, but with a higher affinity to SARS-CoV-2 spike. As a control, the SARS-CoV-2 specific antibody 2-4 only binds only to SARS-CoV-2 spike (FIG. 37C), but not to SARS-CoV spike (FIG. 37D).

FIGS. 38A-B show neutralization assay results. Antibody 2-36 neutralized both SARS-CoV-2 (FIG. 38A) and SARS-CoV (FIG. 38B), as compared to the control antibody CR3022 that neutralized SARS-CoV (FIG. 38B) but only very weakly neutralized SARS-CoV-2 (FIG. 38A); and as compared to the SARS-CoV-2 specific antibody 2-4 that only neutralize SARS-CoV-2 (FIG. 38A).

FIGS. 39A-B show that antibody 2-36 neutralizes SARS-like coronaviruses using hACE2. FIG. 39A shows the lineage of coronavirus, including several from bats and pangolins. FIG. 39B shows that antibody 2-36 could neutralize SARS-CoV and SARS-CoV-2 and their related lineage viruses, but that antibody 2-36 could not neutralize MERS-CoV and 229E, indicating its breadth.

FIGS. 40A-D show the cryo-EM structure of antibody 2-36 complexed with SARS-CoV-2. FIG. 40A shows the cryo-EM structure of 2-36 in complex with SARS-CoV-2 Spike trimer and FIG. 40B further details how 2-36 interacts with spike at the CDR level.

FIG. 40C shows that antibody 2-36 binds to the side of the RBD and can compete with ACE2 for RBD binding, although its epitope does not overlap the ACE2-binding site. FIG. 40D depicts that the spike protein is highly conserved.

FIG. 41 shows a schematic representation of an engineered bispecific antibody combining the binding specificity of two neutralizing antibodies into one.

FIG. 42 shows that the 2-17/1-57 bispecific antibody against SARS-CoV-2 does not enhance potency compared to respective single arm counterpart. Ab X and Ab Y are single arm irrelevant controls. X=PGDM1400 (HIV neutralization Ab). Y=A32 (HIV neutralization Ab).

FIG. 43 shows potent neutralization of bispecific antibody 2-17/2-7 against SARS-CoV-2 compared to respective single arm counterpart. Ab X and Ab Y are single arm irrelevant controls. X=PGDM1400 (HIV neutralization Ab). Y=A32 (HIV neutralization Ab).

FIG. 44 shows potent neutralization of bispecific antibody 2-17/2-15 against SARS-CoV-2 compared to respective single arm counterpart. Ab X and Ab Y are single arm irrelevant controls. X=PGDM1400 (HIV neutralization Ab). Y=A32 (HIV neutralization Ab).

FIG. 45 shows potent neutralization of bispecific antibody 2-17/2-30 against SARS-CoV-2 compared to respective single arm counterpart. Ab X and Ab Y are single arm irrelevant controls. X=PGDM1400 (HIV neutralization Ab). Y=A32 (HIV neutralization Ab).

FIG. 46 shows potent neutralization of bispecific antibody 2-17/4-20 against SARS-CoV-2 compared to respective single arm counterpart. Ab X and Ab Y are single arm irrelevant controls. X=PGDM1400 (HIV neutralization Ab). Y=A32 (HIV neutralization Ab).

FIG. 47 shows potent neutralization of bispecific antibody 5-24/1-57 against SARS-CoV-2 compared to respective single arm counterpart. Ab X and Ab Y are single arm irrelevant controls. X=PGDM1400 (HIV neutralization Ab). Y=A32 (HIV neutralization Ab).

FIG. 48 shows potent neutralization of bispecific antibody 5-24/2-15 against SARS-CoV-2 compared to respective single arm counterpart. Ab X and Ab Y are single arm irrelevant controls. X=PGDM1400 (HIV neutralization Ab). Y=A32 (HIV neutralization Ab).

FIG. 49 shows bispecific antibody 5-24/4-20 against SARS-CoV-2 does not enhance potency (IC₅₀) compared to respective single arm counterpart. Ab X and Ab Y are single arm irrelevant controls. X=PGDM1400 (HIV neutralization Ab). Y=A32 (HIV neutralization Ab).

FIG. 50 shows potent neutralization of bispecific antibody 1-20/1-68 against SARS-CoV-2 compared to respective single arm counterpart. Ab X and Ab Y are single arm irrelevant controls. X=PGDM1400 (HIV neutralization Ab). Y=A32 (HIV neutralization Ab).

FIG. 51 shows potent neutralization of bispecific antibody 1-20/2-17 against SARS-CoV-2 compared to respective single arm counterpart. Ab X and Ab Y are single arm irrelevant controls. X=PGDM1400 (HIV neutralization Ab). Y=A32 (HIV neutralization Ab).

FIG. 52 shows potent neutralization of bispecific antibody 1-20/4-18 against SARS-CoV-2 compared to respective single arm counterpart. Ab X and Ab Y are single arm irrelevant controls. X=PGDM1400 (HIV neutralization Ab). Y=A32 (HIV neutralization Ab).

FIG. 53 shows potent neutralization of bispecific antibody 1-20/5-24 against SARS-CoV-2 compared to respective single arm counterpart. Ab X and Ab Y are single arm irrelevant controls. X=PGDM1400 (HIV neutralization Ab). Y=A32 (HIV neutralization Ab).

FIG. 54 shows potent neutralization against SARS-CoV-2. Bispecific antibodies with 2-17 as a component are more potent than the single arm control (2-17/A32, A32 is a control antibody which shows no activity against SARS-CoV-2), indicating both arms are contributing to the neutralization activity of the bispecific molecule.

FIG. 55 shows potent neutralization against SARS-CoV-2 with bispecific antibodies containing 5-24 as a component. In antibody 5-24/A32, A32 is a control antibody which shows no activity against SARS-CoV-2, indicating both arms are contributing to the neutralization activity of the bispecific molecule.

FIG. 56 shows potent neutralization against SARS-CoV-2 with bispecific antibodies containing 1-20 as a component. In antibody 1-20/A32, A32 is a control antibody which shows no activity against SARS-CoV-2), indicating both arms are contributing to the neutralization activity of the bispecific molecule.

FIGS. 57A-E show bispecific antibodies with 1-20 (RBD) as a parental antibody. FIG. 57A shows virus neutralization with bispecific antibodies 1-20/5-24, 1-20/4-18, 1-20/2-17, and 1-20/1-68. FIG. 57B shows virus neutralization with the bispecific antibody 1-20/5-24 compared to control antibodies. FIG. 57C shows virus neutralization with the bispecific antibody 1-20/4-18 compared to control antibodies. FIG. 57D shows virus neutralization with the bispecific antibody 1-20/2-17 compared to control antibodies. FIG. 57E shows virus neutralization with the bispecific antibody 1-20/1-68 compared to control antibodies.

FIG. 58 shows potencies of bispecific antibodies with 1-20 (RBD) as a parental antibody.

FIGS. 59A-G show bispecific antibodies with 2-17 (NTD) as a parental antibody. FIG. 59A shows virus neutralization with bispecific antibodies 2-17/2-7, 2-17/1-57, 2-36/2-17, 2-17/4-20, 2-17/2-30, and 2-17/2-15. FIG. 59B shows virus neutralization with the bispecific antibody 2-17/2-7 compared to control antibodies. FIG. 59C shows virus neutralization with the bispecific antibody 2-17/1-57 compared to control antibodies. FIG. 59D shows virus neutralization with the bispecific antibody 2-17/2-15 compared to control antibodies. FIG. 59E shows virus neutralization with the bispecific antibody 2-17/4-20 compared to control antibodies. FIG. 59F shows virus neutralization with the bispecific antibody 2-17/2-30 compared to control antibodies. FIG. 59G shows virus neutralization with the bispecific antibody 2-36/2-17 compared to control antibodies.

FIG. 60 shows potencies of bispecific antibodies with 2-17 (NTD) as a parental antibody.

FIGS. 61A-D show bispecific antibodies with 5-24 (NTD) as a parental antibody. FIG. 61A shows virus neutralization with bispecific antibodies 5-24/4-20, 5-24/1-57, 5-24/2-15, and 1-20/5-24. FIG. 61B shows virus neutralization with the bispecific antibody 5-24/2-15 compared to control antibodies. FIG. 61C shows virus neutralization with the bispecific antibody 5-24/1-57 compared to control antibodies. FIG. 61D shows virus neutralization with the bispecific antibody 5-24/4-20 compared to control antibodies.

FIG. 62 shows potencies of bispecific antibodies with 5-24 (NTD) as a parental antibody.

FIGS. 63A-D show bispecific antibodies with 2-36 (RBD) as a parental antibody. FIG. 63A shows virus neutralization with bispecific antibodies 2-36/1-68, 2-36/4-18, and 2-36/2-17. FIG. 63B shows virus neutralization with the bispecific antibody 2-36/2-17 compared to control antibodies. FIG. 63C shows virus neutralization with the bispecific antibody 2-36/1-68 compared to control antibodies. FIG. 63D shows virus neutralization with the bispecific antibody 2-36/4-18 compared to control antibodies.

FIG. 64 shows potencies of bispecific antibodies with 2-36 (RBD) as a parental antibody.

FIGS. 65A-C show bispecific antibodies with 2-30 (RBD) as a parental antibody. FIG. 65A shows virus neutralization with bispecific antibodies 2-30/4-18 and 2-30/1-68.

FIG. 65B shows virus neutralization with the bispecific antibody 2-30/1-68 compared to control antibodies. FIG. 65C shows virus neutralization with the bispecific antibody 2-30/4-18 compared to control antibodies.

FIG. 66 show potencies of bispecific antibodies with 2-30 (RBD) as a parental antibody.

FIGS. 67A-D show comparative evaluations of bispecific antibody 2-17/2-7. FIG. 67A shows the potency of the 2-17/2-7 bispecific antibody to antibodies where one arm resembles the individual parental component of the bispecific antibody while the other arm is an irrelevant control. It also compares the potency of the bispecific antibody to the combination of the two single arm IgG controls. FIG. 67B shows virus neutralization with a combination of the parental antibodies and each of the parental antibodies. FIG. 67C shows virus neutralization with the bispecific antibody 2-17/2-7 compared to a combination of the parental antibodies. FIG. 67D shows potency of combination parental monoclonal antibodies tested each at half of the amount of the corresponding bispecific antibody. As each monoclonal antibody has two identical arms and each bispecific antibody has only one arm for each monoclonal antibody, testing at half of the amount allows for the same concentration of each monoclonal antibody arm to be compared to the bispecific antibody and the monoclonal antibody combination.

FIGS. 68A-D show comparative evaluations of bispecific antibody 1-20/4-18. FIG. 68A compares the potency of the 1-20/4-18 bispecific antibody to an antibody where one arm resembles the individual parental component of the bispecific antibody while the other arm is an irrelevant control. It also compares the potency of the bispecific antibody to the combination of the two single arm IgG controls. FIG. 68B shows virus neutralization with a combination of the parental antibodies and each of the parental antibodies. FIG. 68C shows virus neutralization with the bispecific antibody 1-20/4-18 compared to a combination of the parental antibodies. FIG. 68D shows potency of combination parental monoclonal antibodies tested each at half of the amount of the corresponding bispecific antibody.

FIGS. 69A-D show comparative evaluations of bispecific antibody 1-20/5-24. FIG. 69A compares the potency of the 1-20/5-24 bispecific antibody to an antibody where one arm resembles the individual parental component of the bispecific antibody while the other arm is an irrelevant control. It also compares the potency of the bispecific antibody to the combination of the two single arm IgG controls. FIG. 69B shows virus neutralization with a combination of the parental antibodies and each of the parental antibodies. FIG. 69C shows virus neutralization with the bispecific antibody 1-20/5-24 compared to a combination of the parental antibodies. FIG. 69D shows potency of combination parental monoclonal antibodies tested each at half of the amount of the corresponding bispecific antibody.

FIGS. 70A-D show comparative evaluations of bispecific antibody 2-17/4-20. FIG. 70A compares the potency of the 2-17/4-20 bispecific antibody to an antibody where one arm resembles the individual parental component of the bispecific antibody while the other arm is an irrelevant control. It also compares the potency of the bispecific antibody to the combination of the two single arm IgG controls. FIG. 70B shows virus neutralization with a combination of the parental antibodies and each of the parental antibodies. FIG. 70C shows virus neutralization with the bispecific antibody 2-17/4-20 compared to a combination of the parental antibodies. FIG. 70D shows potency of combination parental monoclonal antibodies tested each at half of the amount of the corresponding bispecific antibody.

FIGS. 71A-B show in vitro potency of bispecific antibodies against SARS-CoV-2 authentic virus, strain USA/WA1. FIG. 71A shows neutralization for ten bispecific antibodies with IC₉₀ concentrations between 0.004 μg/ml and 0.025 μg/ml. FIG. 71B shows neutralization for nine bispecific antibodies with IC₉₀ concentrations between 0.026 μg/ml and 0.046 μg/ml. Authentic virus is another usage term for “infectious” virus. The term can also refer to “live virus”. The USA/WA1 strain is the first strain isolated in the United States and is provided by BEI resources.

FIG. 72 shows in vitro potency of bispecific antibodies presented from smallest to highest IC₉₀ concentration. Highlighted antibodies were tested with controls.

FIGS. 73A-B show in vitro potency of bispecific antibodies showing IC₉₀ and IC₅₀ concentrations. FIG. 73A shows in vitro potency of bispecific antibodies showing IC₉₀ and IC₅₀ concentrations relative to a 0.05 μg/ml threshold. FIG. 73B shows in vitro potency of bispecific antibodies showing IC₉₀ and IC₅₀ concentrations relative to a 0.01 μg/ml threshold.

FIGS. 74A-D show comparative evaluation of the bispecific antibody 1-57/1-68. FIG. 74A shows virus neutralization with the bispecific antibody 1-57/1-68 compared to two control bispecific antibodies, each comprising a parental antibody arm and a control antibody arm, and compared to a mix of the two control bispecific antibodies. AbX is an irrelevant control antibody arm, wherein AbX is PGDM1400. FIG. 74B shows virus neutralization with a combination of the parental antibodies and each of the parental antibodies. FIG. 74C shows virus neutralization with the bispecific antibody 1-57/1-68 compared to a combination of the parental antibodies. FIG. 74D shows potency of the bispecific antibody 1-57/1-68 and a mix of each parental monoclonal antibodies tested each at half of the amount of the corresponding bispecific antibody. The comparative data are from a single experiment, while, the data in the summary table are averaged from multiple repeats.

FIGS. 75A-D show comparative evaluation of the bispecific antibody 2-17/2-7. FIG. 75A shows virus neutralization with the bispecific antibody 2-17/2-7 compared to two control bispecific antibodies, each comprising a parental antibody arm and a control antibody arm, and compared to a mix of the two control bispecific antibodies. AbX and AbY are irrelevant control antibody arms, wherein AbX is PGDM1400 and AbY is A32. FIG. 75B shows virus neutralization with a combination of the parental antibodies and each of the parental antibodies. FIG. 75C shows virus neutralization with the bispecific antibody 2-17/2-7 compared to a combination of the parental antibodies. FIG. 75D shows potency of the bispecific antibody 2-17/2-7 and a mix of each parental monoclonal antibodies tested each at half of the amount of the corresponding bispecific antibody. The comparative data are from a single experiment, while, the data in the summary table are averaged from multiple repeats.

FIGS. 76A-D show comparative evaluation of the bispecific antibody 1-20/4-18. FIG. 76A shows virus neutralization with the bispecific antibody 1-20/4-18 compared to two control bispecific antibodies, each comprising a parental antibody arm and a control antibody arm, and compared to a mix of the two control bispecific antibodies. AbX and AbY are irrelevant control antibody arms, wherein AbX is PGDM1400 and AbY is A32. FIG. 76B shows virus neutralization with a combination of the parental antibodies and each of the parental antibodies. FIG. 76C shows virus neutralization with the bispecific antibody 1-20/4-18 compared to a combination of the parental antibodies. FIG. 76D shows potency of the bispecific antibody 1-20/4-18 and a mix of each parental monoclonal antibodies tested each at half of the amount of the corresponding bispecific antibody. The comparative data are from a single experiment, while, the data in the summary table are averaged from multiple repeats.

FIGS. 77A-D show comparative evaluation of the bispecific antibody 1-20/5-24. FIG. 77A shows virus neutralization with the bispecific antibody 1-20/5-24 compared to two control bispecific antibodies, each comprising a parental antibody arm and a control antibody arm, and compared to a mix of the two control bispecific antibodies. AbX and AbY are irrelevant control antibody arms, wherein AbX is PGDM1400 and AbY is A32. FIG. 77B shows virus neutralization with a combination of the parental antibodies and each of the parental antibodies. FIG. 77C shows virus neutralization with the bispecific antibody 1-20/5-24 compared to a combination of the parental antibodies. FIG. 77D shows potency of the bispecific antibody 1-20/5-24 and a mix of each parental monoclonal antibodies tested each at half of the amount of the corresponding bispecific antibody. The comparative data are from a single experiment, while, the data in the summary table are averaged from multiple repeats.

FIGS. 78A-D show comparative evaluation of the bispecific antibody 2-30/1-68. FIG. 78A shows virus neutralization with the bispecific antibody 2-30/1-68 compared to two control bispecific antibodies, each comprising a parental antibody arm and a control antibody arm, and compared to a mix of the two control bispecific antibodies. AbX is an irrelevant control antibody arms, wherein AbX is PGDM1400. FIG. 78B shows virus neutralization with a combination of the parental antibodies and each of the parental antibodies. FIG. 78C shows virus neutralization with the bispecific antibody 2-30/1-68 compared to a combination of the parental antibodies. FIG. 78D shows potency of the bispecific antibody 2-30/1-68 and a mix of each parental monoclonal antibodies tested each at half of the amount of the corresponding bispecific antibody. The comparative data are from a single experiment, while, the data in the summary table are averaged from multiple repeats.

FIGS. 79A-D show comparative evaluation of the bispecific antibody 2-7/4-8 (39/51). FIG. 79A shows virus neutralization with the bispecific antibody 2-7/4-8 (39/51) compared to two control bispecific antibodies, each comprising a parental antibody arm and a control antibody arm, and compared to a mix of the two control bispecific antibodies. AbX is an irrelevant control antibody arms, wherein AbX is PGDM1400. FIG. 79B shows virus neutralization with a combination of the parental antibodies and each of the parental antibodies. FIG. 79C shows virus neutralization with the bispecific antibody 2-7/4-8 (39/51) compared to a combination of the parental antibodies. FIG. 79D shows potency of the bispecific antibody 2-7/4-8 (39/51) and a mix of each parental monoclonal antibodies tested each at half of the amount of the corresponding bispecific antibody. The comparative data are from a single experiment, while, the data in the summary table are averaged from multiple repeats.

FIGS. 80A-D show comparative evaluation of the bispecific antibody 1-57/1-87. FIG. 80A shows virus neutralization with the bispecific antibody 1-57/1-87 compared to two control bispecific antibodies, each comprising a parental antibody arm and a control antibody arm, and compared to a mix of the two control bispecific antibodies. AbX is an irrelevant control antibody arms, wherein AbX is PGDM1400. FIG. 80B shows virus neutralization with a combination of the parental antibodies and each of the parental antibodies. FIG. 80C shows virus neutralization with the bispecific antibody 1-57/1-87 compared to a combination of the parental antibodies. FIG. 80D shows potency of the bispecific antibody 1-57/1-87 and a mix of each parental monoclonal antibodies tested each at half of the amount of the corresponding bispecific antibody. The comparative data are from a single experiment, while, the data in the summary table are averaged from multiple repeats.

FIG. 81 shows potency of additional bispecific antibodies.

FIGS. 82A-B show neutralizing activities of engineered bispecific antibodies. FIG. 82A shows neutralizing activities against wild-type virus (WA1) at various antibody concentrations for bispecific antibodies 2-17/2-7, 1-57/5-7, 5-7/1-57, 2-7/5-7, 5-7/2-7. FIG. 82B shows antibody potency (IC50 and IC90) against wild-type virus (WA1) for bispecific antibodies 2-17/2-7, 1-57/5-7, 5-7/1-57, 2-7/5-7, 5-7/2-7.

FIGS. 83A-B show 2-7/5-7 bispecific antibody and parental antibodies activities. FIG. 83A shows neutralizing activities against wild-type virus (WA1) at various antibody concentrations for bispecific antibody 2-7/5-7, parental antibodies 2-7, 5-7, and a combination of 2-7 and 5-7 parental antibodies. FIG. 83B shows IC₅₀ concentrations for bispecific antibody 2-7/5-7, parental antibodies 2-7, 5-7, and a combination of 2-7 and 5-7 parental antibodies.

FIGS. 84A-B show activities of bispecific antibody 2-7/5-7 against SARS-CoV-2 wild-type (WA1) and variant strains B1.1.7 (UK), B1.526 (New York), B1.351 (South Africa) and P.1 (Brazil). FIG. 84A shows neutralization activity of bispecific antibody 2-7/5-7 against various SARS-CoV-2 strains at different antibody concentrations. FIG. 84B shows IC₅₀ and IC₉₀ concentrations for bispecific antibody 2-7/5-7 against various SARS-CoV-2 strains.

FIGS. 85A-D show neutralization of pseudoviruses having mutations associated with SARS-CoV-2 variants with the bispecific 2-7/5-7 antibody. FIG. 85A shows antibody neutralization curves against circulating SARS-CoV-2 virus variants. FIG. 85B shows antibody potency against circulating SARS-CoV-2 virus variants. FIG. 85C shows antibody neutralization curves against SARS-CoV-2 variants with high frequency mutations. FIG. 85D shows antibody potency against SARS-CoV-2 variants with high frequency mutations.

FIGS. 86A-F show neutralization of pseudoviruses with SARS-CoV-2 mutations corresponding to “variants of concern” using the bispecific antibody 2-7/5-7. FIG. 86A shows antibody neutralization curves against B.1.1.7 (UK virus variant) with NTD mutations. FIG. 86B shows antibody potency against B.1.1.7 (UK virus variant) with NTD mutations. FIG. 86C shows antibody neutralization curves against B.1.352 (SA) virus variant with NTD mutations. FIG. 86D shows antibody potency against B.1.352 (SA) virus variant with NTD mutations. FIG. 86E shows antibody neutralization curves against P.1 (BZ) virus variant with NTD mutations. FIG. 86F shows antibody potency against P.1 (BZ) virus variant with NTD mutations.

FIGS. 87A-F show neutralization of pseudoviruses with SARS-CoV-2 mutations corresponding to “variants of interest” using the bispecific antibody 2-7/5-7. FIG. 87A shows antibody neutralization curves against B.1.427 (CA) virus variant with NTD mutations. FIG. 87B shows antibody potency against B.1.427 (CA) virus variant with NTD mutations. FIG. 87C shows antibody neutralization curves against B.1.526 (NY) variant with NTD mutations.

FIG. 87D shows antibody potency against B.1.526 (NY) variant with NTD mutations. FIG. 87E show antibody neuralization curves against NJ variant with NTD mutations. FIG. 87F show antibody potency against NJ variant with NTD mutations.

FIG. 88 shows summary of bispecific antibody 2-7/5-7 activity against variants. IC50 is shown in circles. IC90 is shown in squares.

FIGS. 89A-C shows neutralization activity of bispecific antibody 2-7/5-7 and parental 2-7 and 5-7 monoclonal antibodies against SARS-CoV-2 variants (live viruses). FIG. 89A shows the neutralization curve for bispecific antibody 2-7/5-7. FIG. 89B shows the neutralization curve for parental antibody 2-7. FIG. 89C shows the neutralization curve for parental antibody 5-7.

FIGS. 90A-D show virus neutralization with monoclonal antibody 2-36. FIG. 90A shows screening of transfection supernatant for neutralization activity against SARS-CoV-2 pseudovirus. FIG. 90B shows screening of transfection supernatant for neutralization activity against SARS-CoV pseudovirus. FIG. 90C shows 2-36 neutralization IC50 (μg/mL) against SARS-CoV-2 pseudovirus (PV) and live virus (LV). FIG. 90D shows 2-36 neutralization IC50 (μg/mL) against SARS-CoV pseudovirus (PV) and live virus (LV).

FIGS. 91A-B show binding of monoclonal antibody 2-36 to SARS-CoV-2 (FIG. 91A) and SARS-CoV (FIG. 91B) spike as determined by ELISA.

FIGS. 92A-H show binding affinity for monoclonal antibodies 2-36 and 2-4 to SARS-CoV-2 and SARS-CoV viruses as measured by SPR. FIG. 92A shows 2-36 binding affinity to SARS-CoV-2 spike protein. FIG. 92B shows 2-4 binding affinity to SARS-CoV-2 spike protein. FIG. 92C shows 2-36 binding affinity to SARS-CoV spike protein. FIG. 92D shows 2-4 binding affinity to SARS-CoV spike protein. FIG. 92E shows 2-36 binding affinity to SARS-CoV-2 receptor binding domain (RBD). FIG. 92F shows 2-4 binding affinity to SARS-CoV-2 RBD. FIG. 92G shows 2-36 binding affinity to SARS-CoV RBD. FIG. 92H shows 2-4 binding affinity to SARS-CoV RBD.

FIGS. 93A-B show binding to SARS-CoV-2 spike protein. FIG. 93A shows that binding of monoclonal antibody 2-36 to SARS-CoV-2 spike is inhibited by CR3022. FIG. 93B shows that monoclonal antibody 2-36 inhibits hACE2 binding to SARS-CoV-2 spike protein.

FIG. 94 shows conservation analysis on the RBD among SARS CoV-2, SARS CoV-1, Bat CoVs, and Human CoVs virus strains. The analysis shows that the 2-36 binding site is highly conserved; with regions of high conservation in grey and low conservation in red. The 2-36 binding site is outlined in blue.

FIGS. 95A-D show that monoclonal antibody 2-36 neutralizes SARS-CoV-2 variants and SARS-like coronaviruses using hACE2. FIG. 95A shows neutralization against live variants. FIG. 95B shows neutralization against pseudovariants. FIG. 95C shows evolution tree of viruses. FIG. 95D shows antibody potency against viruses.

FIGS. 96A-D shows that monoclonal antibody 2-36 neutralizes SARS-like coronaviruses using hACE2. FIG. 96A shows virus neutralization with 2-36 antibody. FIG. 96B shows virus neutralization with S309 antibody. FIG. 96C shows virus neutralization with COVA1-16 antibody. FIG. 96D shows virus neutralization with CR3022 antibody.

FIGS. 97A-D show in vitro selection of 2-36 antibody escape mutations. FIG. 97A shows evolution of escape mutations during in vitro passage of SARS-CoV-2 USA/WA1 in Vero E6 cells in the presence of 2-36. FIG. 97B shows 2-36 neutralization activity tested against virus passages. FIG. 97C shows spike protein with selected mutation localized. FIG. 97D shows that the selected escape mutations were introduced into pseudoviruses and then 2-36 neutralization activity was tested against these viruses.

### DETAILED DESCRIPTION OF THE INVENTION

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

As used herein, the term “subject” refers to a vertebrate animal. In one embodiment, the subject is a mammal or a mammalian species. In one embodiment, the subject is a human. In one embodiment, the subject is a healthy human adult. In other embodiments, the subject is a non-human vertebrate animal, including, without limitation, non-human primates, laboratory animals, livestock, racehorses, domesticated animals, and non-domesticated animals. In one embodiment, the term “human subjects” means a population of healthy human adults.

As used herein, the term “patient” refers to a human or animal.

The term “mammal” includes, but is not limited to, a human, mouse, rat, guinea pig, dog, cat, horse, cow, pig, or non-human primate, such as a monkey, chimpanzee, baboon or rhesus. In one embodiment, the mammal is a human.

As used herein, the term “therapeutically effective” includes prophylaxis, as well as treatment of a subject having suspected of having a viral infection.

In certain aspects, the invention provides a bispecific molecule comprising a first polypeptide chain and a second polypeptide chain, wherein: the first polypeptide chain comprises: a first light chain comprising a variable domain and a constant domain; a first heavy chain comprising a variable domain and a constant domain; the second polypeptide chain comprises: a second light chain comprising a variable domain and a constant domain; and a second heavy chain comprising a variable domain and a constant domain.

In some embodiments, the first polypeptide chain and the second polypeptide chain are covalently bonded to one another. In some embodiments, the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 59 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 63. In some embodiments, the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 60 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 64. In some embodiments, the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 61 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 65. In some embodiments, the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 62 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 66.

In some embodiments, the first heavy chain comprises SEQ ID NO: 59 or SEQ ID NO: 63. In some embodiments, the first light chain comprises SEQ ID NO: 60 or SEQ ID NO: 64. In some embodiments, the second heavy chain comprises SEQ ID NO: 61 or SEQ ID NO: 65. In some embodiments, the second light chain comprises SEQ ID NO: 62 or SEQ ID NO: 66.

In some embodiments, the variable domain of the first heavy chain comprises CDR1, CDR2, and CDR3 of SEQ ID NO: 59 or CDR1, CDR2, and CDR3 of SEQ ID NO: 63. In some embodiments, the variable domain of the first light chain comprises CDR1, CDR2, and CDR3 of SEQ ID NO: 60 or CDR1, CDR2, and CDR3 of SEQ ID NO: 64. In some embodiments, the variable domain of the second heavy chain comprises CDR1, CDR2, and CDR3 of SEQ ID NO: 61 or CDR1, CDR2, and CDR3 of SEQ ID NO: 65. In some embodiments, the variable domain of the second light chain comprises CDR1, CDR2, and CDR3 SEQ ID NO: 62 or CDR1, CDR2, and CDR3 of SEQ ID NO: 66.

In some embodiments, the variable domain of the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27. In some embodiments, the variable domain of the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28. In some embodiments, the variable domain of the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13. In some embodiments, the variable domain of the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14.

In some embodiments, the variable domain of the first heavy chain comprises SEQ ID NO: 13 or SEQ ID NO: 27. In some embodiments, the variable domain of the first light chain comprises SEQ ID NO: 14 or SEQ ID NO: 28. In some embodiments, the variable domain of the second heavy chain comprises SEQ ID NO: 27 or SEQ ID NO: 13. In some embodiments, the variable domain of the second light chain comprises SEQ ID NO: 28 or SEQ ID NO: 14.

In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 59, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 60, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 61, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 62. In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 63, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 64, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 65, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 66. In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 13, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28.

In some embodiments, the molecule comprises SEQ ID NOs: 59, 60, 61, and 62. In some embodiments, the molecule comprises SEQ ID NOs: 63, 64, 65, and 66. In some embodiments, the molecule comprises SEQ ID NOs: 13, 14, 27, and 28.

In some embodiments, the molecule further comprises one or more disulfide bonds. In some embodiments, the molecule further comprises one or more linker sequences. In some embodiments, the molecule is capable of neutralizing a coronavirus. In some embodiments, the coronavirus is a SARS-CoV-2 virus strain. In some embodiments, the molecule specifically recognizes a first epitope and a second epitope on a SARS-CoV-2 particle. In some embodiments, the first epitope comprises at least a portion of a receptor binding domain (RBD) on an S protein of a SARS-CoV-2 particle. In some embodiments, the second epitope comprises at least a portion of a N-terminal domain (NTD) on an S protein of a SARS-CoV-2 particle.

In certain aspects, the invention provides a method of treating a COVID-19 infection in a subject in need thereof, the method comprising administering to the subject a composition comprising a bispecific molecule, wherein the bispecific molecule comprises a first polypeptide chain and a second polypeptide chain, wherein: the first polypeptide chain comprises: a first light chain comprising a variable domain and a constant domain; a first heavy chain comprising a variable domain and a constant domain; the second polypeptide chain comprises: a second light chain comprising a variable domain and a constant domain; and a second heavy chain comprising a variable domain and a constant domain.

In some embodiments, the first polypeptide chain and the second polypeptide chain are covalently bonded to one another. In some embodiments, the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 59 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 63. In some embodiments, the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 60 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 64. In some embodiments, the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 61 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 65. In some embodiments, the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 62 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 66.

In some embodiments, the first heavy chain comprises SEQ ID NO: 59 or SEQ ID NO: 63. In some embodiments, the first light chain comprises SEQ ID NO: 60 or SEQ ID NO: 64. In some embodiments, the second heavy chain comprises SEQ ID NO: 61 or SEQ ID NO: 65. In some embodiments, the second light chain comprises SEQ ID NO: 62 or SEQ ID NO: 66. In some embodiments, the variable domain of the first heavy chain comprises CDR1, CDR2, and CDR3 of SEQ ID NO: 59 or CDR1, CDR2, and CDR3 of SEQ ID NO: 63. In some embodiments, the variable domain of the first light chain comprises CDR1, CDR2, and CDR3 of SEQ ID NO: 60 or CDR1, CDR2, and CDR3 of SEQ ID NO: 64. In some embodiments, the variable domain of the second heavy chain comprises CDR1, CDR2, and CDR3 of SEQ ID NO: 61 or CDR1, CDR2, and CDR3 of SEQ ID NO: 65. In some embodiments, the variable domain of the second light chain comprises CDR1, CDR2, and CDR3 SEQ ID NO: 62 or CDR1, CDR2, and CDR3 of SEQ ID NO: 66.

In some embodiments, the variable domain of the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27. In some embodiments, the variable domain of the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28. In some embodiments, the variable domain of the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13. In some embodiments, the variable domain of the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14.

In some embodiments, the variable domain of the first heavy chain comprises SEQ ID NO: 13 or SEQ ID NO: 27. In some embodiments, the variable domain of the first light chain comprises SEQ ID NO: 14 or SEQ ID NO: 28. In some embodiments, the variable domain of the second heavy chain comprises SEQ ID NO: 27 or SEQ ID NO: 13. In some embodiments, the variable domain of the second light chain comprises SEQ ID NO: 28 or SEQ ID NO: 14.

In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 59, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 60, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 61, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 62. In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 63, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 64, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 65, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 66. In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 13, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28.

In some embodiments, the molecule comprises SEQ ID NOs: 59, 60, 61, and 62. In some embodiments, the molecule comprises SEQ ID NOs: 63, 64, 65, and 66. In some embodiments, the molecule comprises SEQ ID NOs: 13, 14, 27, and 28.

In some embodiments, the molecule further comprises one or more disulfide bonds. In some embodiments, the molecule further comprises one or more linker sequences. In some embodiments, the molecule is capable of neutralizing a coronavirus. In some embodiments, the coronavirus is a SARS-CoV-2 virus strain. In some embodiments, the molecule specifically recognizes a first epitope and a second epitope on a SARS-CoV-2 particle. In some embodiments, the first epitope comprises at least a portion of a receptor binding domain (RBD) on a spike protein of a SARS-CoV-2 particle. In some embodiments, the second epitope comprises at least a portion of a N-terminal domain (NTD) on a spike protein of a SARS-CoV-2 particle. In some embodiments, the composition is administered in combination with a second therapeutic agent.

In certain aspects, the invention provides a method of preventing a COVID-19 infection in a subject in need thereof, the method comprising administering to the subject a composition comprising a bispecific molecule, wherein the bispecific molecule comprises a first polypeptide chain and a second polypeptide chain, wherein: the first polypeptide chain comprises: a first light chain comprising a variable domain and a constant domain; a first heavy chain comprising a variable domain and a constant domain; the second polypeptide chain comprises: a second light chain comprising a variable domain and a constant domain; and a second heavy chain comprising a variable domain and a constant domain.

In some embodiments, the first polypeptide chain and the second polypeptide chain are covalently bonded to one another. In some embodiments, the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 59 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 63. In some embodiments, the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 60 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 64. In some embodiments, the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 61 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 65. In some embodiments, the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 62 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 66.

In some embodiments, the first heavy chain comprises SEQ ID NO: 59 or SEQ ID NO: 63. In some embodiments, the first light chain comprises SEQ ID NO: 60 or SEQ ID NO: 64. In some embodiments, the second heavy chain comprises SEQ ID NO: 61 or SEQ ID NO: 65. In some embodiments, the second light chain comprises SEQ ID NO: 62 or SEQ ID NO: 66.

In some embodiments, the variable domain of the first heavy chain comprises CDR1, CDR2, and CDR3 of SEQ ID NO: 59 or CDR1, CDR2, and CDR3 of SEQ ID NO: 63. In some embodiments, the variable domain of the first light chain comprises CDR1, CDR2, and CDR3 of SEQ ID NO: 60 or CDR1, CDR2, and CDR3 of SEQ ID NO: 64. In some embodiments, the variable domain of the second heavy chain comprises CDR1, CDR2, and CDR3 of SEQ ID NO: 61 or CDR1, CDR2, and CDR3 of SEQ ID NO: 65. In some embodiments, the variable domain of the second light chain comprises CDR1, CDR2, and CDR3 SEQ ID NO: 62 or CDR1, CDR2, and CDR3 of SEQ ID NO: 66.

In some embodiments, the variable domain of the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27. In some embodiments, the variable domain of the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28. In some embodiments, the variable domain of the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13. In some embodiments, the variable domain of the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14.

In some embodiments, the variable domain of the first heavy chain comprises SEQ ID NO: 13 or SEQ ID NO: 27. In some embodiments, the variable domain of the first light chain comprises SEQ ID NO: 14 or SEQ ID NO: 28. In some embodiments, the variable domain of the second heavy chain comprises SEQ ID NO: 27 or SEQ ID NO: 13. In some embodiments, the variable domain of the second light chain comprises SEQ ID NO: 28 or SEQ ID NO: 14.

In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 59, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 60, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 61, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 62. In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 63, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 64, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 65, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 66. In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 13, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28.

In some embodiments, the molecule comprises SEQ ID NOs: 59, 60, 61, and 62. In some embodiments, the molecule comprises SEQ ID NOs: 63, 64, 65, and 66. In some embodiments, the molecule comprises SEQ ID NOs: 13, 14, 27, and 28.

In some embodiments, the molecule further comprises one or more disulfide bonds. In some embodiments, the molecule further comprises one or more linker sequences. In some embodiments, the molecule is capable of neutralizing a coronavirus. In some embodiments, the coronavirus is a SARS-CoV-2 virus strain. In some embodiments, the molecule specifically recognizes a first epitope and a second epitope on a SARS-CoV-2 particle. In some embodiments, the first epitope comprises at least a portion of a receptor binding domain (RBD) on an S protein of a SARS-CoV-2 particle. In some embodiments, the second epitope comprises at least a portion of a N-terminal domain (NTD) on an S protein of a SARS-CoV-2 particle. In some embodiments, the composition is administered in combination with a second therapeutic agent.

In certain aspects, the invention provides a synthesized monoclonal antibody, or a functional fragment thereof, comprising a heavy chain having a variable domain and a constant domain and a light chain having a variable domain and a constant domain, wherein the antibody binds an antigen on a coronavirus particle.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the heavy chain variable domain comprises SEQ ID NO: 15. In some embodiments, the light chain variable domain comprises SEQ ID NO: 16.

In some embodiments, the antibody comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15 and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the antibody comprises SEQ ID NO: 15 and SEQ ID NO: 16.

In some embodiments, the heavy chain variable domain comprises the CDR1, CDR2, and CDR3 regions of SEQ ID NO: 15. In some embodiments, the light chain variable domain comprises the CDR1, CDR2, and CDR3 regions of SEQ ID NO: 16.

In some embodiments, the antibody neutralizes a coronavirus. In some embodiments, the coronavirus is SARS-CoV-2. In some embodiments, the antibody specifically binds an epitope on the surface of the coronavirus. In some embodiments, the epitope comprises at least a portion of a coronavirus spike protein. In some embodiments, the at least a portion of a coronavirus spike protein comprises a receptor binding domain (RBD) of a spike protein. In some embodiments, the coronavirus is a SARS-CoV-2 coronavirus.

In certain aspects, the invention provides a method of treating a COVID-19 infection in a subject in need thereof, the method comprising administering to the subject a therapeutically effective amount of a synthesized monoclonal antibody, or a functional fragment thereof, comprising a heavy chain having a variable domain and a constant domain and a light chain having a variable domain and a constant domain, wherein the antibody binds an antigen on a coronavirus particle.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the heavy chain variable domain comprises SEQ ID NO: 15. In some embodiments, the light chain variable domain comprises SEQ ID NO: 16.

In some embodiments, the antibody comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15 and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the antibody comprises SEQ ID NO: 15 and SEQ ID NO: 16.

In some embodiments, the heavy chain variable domain comprises the CDR1, CDR2, and CDR3 regions of SEQ ID NO: 15. In some embodiments, the light chain variable domain comprises the CDR1, CDR2, and CDR3 regions of SEQ ID NO: 16.

In some embodiments, the antibody neutralizes a coronavirus. In some embodiments, the coronavirus is SARS-CoV-2. In some embodiments, the antibody neutralizes a coronavirus. In some embodiments, the coronavirus is SARS-CoV-2. In some embodiments, the antibody specifically binds an epitope on the surface of the coronavirus. In some embodiments, the epitope comprises at least a portion of a coronavirus spike protein. In some embodiments, the at least a portion of a coronavirus spike protein comprises a receptor binding domain (RBD) of a spike protein. In some embodiments, the coronavirus is a SARS-CoV-2 coronavirus. In some embodiments, the antibody is administered in combination with a second pharmaceutical agent.

In certain aspects, the invention provides a method of preventing a COVID-19 infection in a subject in need thereof, the method comprising administering to the subject a therapeutically effective amount of a synthesized monoclonal antibody, or a functional fragment thereof, comprising a heavy chain having a variable domain and a constant domain and a light chain having a variable and a constant domain, wherein the antibody binds an antigen on a coronavirus particle.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the heavy chain variable domain comprises SEQ ID NO: 15. In some embodiments, the light chain variable domain comprises SEQ ID NO: 16.

In some embodiments, the antibody comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15 and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the antibody comprises SEQ ID NO: 15 and SEQ ID NO: 16.

In some embodiments, the heavy chain variable domain comprises the CDR1, CDR2, and CDR3 regions of SEQ ID NO: 15. In some embodiments, the light chain variable domain comprises the CDR1, CDR2, and CDR3 regions of SEQ ID NO: 16.

In some embodiments, the antibody neutralizes a coronavirus. In some embodiments, the coronavirus is SARS-CoV-2. In some embodiments, the antibody neutralizes a coronavirus. In some embodiments, the coronavirus is SARS-CoV-2. In some embodiments, the antibody specifically binds an epitope on the surface of the coronavirus. In some embodiments, the epitope comprises at least a portion of a coronavirus spike protein. In some embodiments, the at least a portion of a coronavirus spike protein comprises a receptor binding domain (RBD) of a spike protein. In some embodiments, the coronavirus is a SARS-CoV-2 coronavirus. In some embodiments, the antibody is administered in combination with a second pharmaceutical agent.

In certain aspects, the invention provides a synthesized monoclonal antibody, or a functional fragment thereof, comprising a heavy chain having a variable domain and a constant domain and a light chain having a variable domain and a constant domain, wherein the antibody binds an antigen on a coronavirus particle.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 29, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 31.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 33, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 39, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 45.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 30, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 32.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 34, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 40, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 46.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 29 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 30.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 31 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 32. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 33 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 34. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 39 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 40. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 45 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 46.

In some embodiments, the heavy chain variable domain comprises SEQ ID NO: 3 and the light chain variable domain comprises SEQ ID NO: 4. In some embodiments, the heavy chain variable domain comprises SEQ ID NO: 7 and the light chain variable domain comprises SEQ ID NO: 8. In some embodiments, the heavy chain variable domain comprises SEQ ID NO: 9 and the light chain variable domain comprises SEQ ID NO: 10. In some embodiments, the heavy chain variable domain comprises SEQ ID NO: 11 and the light chain variable domain comprises SEQ ID NO: 12. In some embodiments, the heavy chain variable domain comprises SEQ ID NO: 17 and the light chain variable domain comprises SEQ ID NO: 18. In some embodiments, the heavy chain variable domain comprises SEQ ID NO: 19 and the light chain variable domain comprises SEQ ID NO: 20. In some embodiments, the heavy chain variable domain comprises SEQ ID NO: 21 and the light chain variable domain comprises SEQ ID NO: 22. In some embodiments, the heavy chain variable domain comprises SEQ ID NO: 25 and the light chain variable domain comprises SEQ ID NO: 26. In some embodiments, the heavy chain variable domain comprises SEQ ID NO: 29 and the light chain variable domain comprises SEQ ID NO: 30. In some embodiments, the heavy chain variable domain comprises SEQ ID NO: 31 and the light chain variable domain comprises SEQ ID NO: 32. In some embodiments, the heavy chain variable domain comprises SEQ ID NO: 33 and the light chain variable domain comprises SEQ ID NO: 34. In some embodiments, the heavy chain variable domain comprises SEQ ID NO: 37 and the light chain variable domain comprises SEQ ID NO: 38. In some embodiments, the heavy chain variable domain comprises SEQ ID NO: 39 and the light chain variable domain comprises SEQ ID NO: 40. In some embodiments, the heavy chain variable domain comprises SEQ ID NO: 43 and the light chain variable domain comprises SEQ ID NO: 44. In some embodiments, the heavy chain variable domain comprises SEQ ID NO: 45 and the light chain variable domain comprises SEQ ID NO: 46.

In some embodiments, the heavy chain variable domain comprises the CDR1, CDR2, and CDR3 regions of SEQ ID NO: 3; the CDR1, CDR2, and CDR3 regions of SEQ ID NO: 7; the CDR1, CDR2, and CDR3 regions of SEQ ID NO: 9; the CDR1, CDR2, and CDR3 regions of SEQ ID NO: 11; or the CDR1, CDR2, and CDR3 regions of SEQ ID NO: 17. In some embodiments, the heavy chain variable domain comprises the CDR1, CDR2, and CDR3 regions of SEQ ID NO: 19; the CDR1, CDR2, and CDR3 regions of SEQ ID NO: 21; the CDR1, CDR2, and CDR3 regions of SEQ ID NO: 25; the CDR1, CDR2, and CDR3 regions of SEQ ID NO: 29; or the CDR1, CDR2, and CDR3 regions of SEQ ID NO: 31. In some embodiments, the heavy chain variable domain comprises the CDR1, CDR2, and CDR3 regions of SEQ ID NO: 33; the CDR1, CDR2, and CDR3 regions of SEQ ID NO: 37; the CDR1, CDR2, and CDR3 regions of SEQ ID NO: 39; the CDR1, CDR2, and CDR3 regions of SEQ ID NO: 43; or the CDR1, CDR2, and CDR3 regions of SEQ ID NO: 45. In some embodiments, the light chain variable domain comprises the CDR1, CDR2, and CDR3 regions of SEQ ID NO: 4; the CDR1, CDR2, and CDR3 regions of SEQ ID NO: 8; the CDR1, CDR2, and CDR3 regions of SEQ ID NO: 10; the CDR1, CDR2, and CDR3 regions of SEQ ID NO: 12; or the CDR1, CDR2, and CDR3 regions of SEQ ID NO: 18. In some embodiments, the light chain variable domain comprises the CDR1, CDR2, and CDR3 regions of SEQ ID NO: 20; the CDR1, CDR2, and CDR3 regions of SEQ ID NO: 22; the CDR1, CDR2, and CDR3 regions of SEQ ID NO: 26; the CDR1, CDR2, and CDR3 regions of SEQ ID NO: 30; or the CDR1, CDR2, and CDR3 regions of SEQ ID NO: 32. In some embodiments, the light chain variable domain comprises the CDR1, CDR2, and CDR3 regions of SEQ ID NO: 34; the CDR1, CDR2, and CDR3 regions of SEQ ID NO: 38; the CDR1, CDR2, and CDR3 regions of SEQ ID NO: 40; the CDR1, CDR2, and CDR3 regions of SEQ ID NO: 44; or the CDR1, CDR2, and CDR3 regions of SEQ ID NO: 46.

In some embodiments, the antibody neutralizes a coronavirus. In some embodiments, the coronavirus is a SARS-CoV-2 virus. In some embodiments, the coronavirus is a SARS-CoV-2 variant strain. In some embodiments, the variant strain is selected from the group consisting of WA1, B.1.1.7, B1.526, B1.351, P1, B1.352, and B.1.427.

In some embodiments, the antibody specifically binds an epitope on the surface of the coronavirus. In some embodiments, the epitope comprises at least a portion of a coronavirus spike protein. In some embodiments, the at least a portion of a coronavirus spike protein comprises a receptor binding domain (RBD) of a spike protein. In some embodiments, the at least a portion of a coronavirus spike protein comprises a N-Terminal domain (NTD) of a spike protein. In some embodiments, the coronavirus is a SARS-CoV-2 coronavirus.

In certain aspects, the invention provides a method of treating or preventing a COVID-19 infection in a subject in need thereof, the method comprising administering to the subject a therapeutically effective amount of a synthesized monoclonal antibody, or a functional fragment thereof, comprising a heavy chain having a variable domain and a constant domain and a light chain having a variable domain and a constant domain, wherein the antibody binds an antigen on a coronavirus particle.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 29, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 31.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 33, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 39, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 45.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 30, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 32.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 34, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 40, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 46.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 29 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 30. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 31 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 32. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 33 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 34. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 39 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 40. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 45 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 46.

In some embodiments, the heavy chain variable domain comprises SEQ ID NO: 3 and the light chain variable domain comprises SEQ ID NO: 4. In some embodiments, the heavy chain variable domain comprises SEQ ID NO: 7 and the light chain variable domain comprises SEQ ID NO: 8. In some embodiments, the heavy chain variable domain comprises SEQ ID NO: 9 and the light chain variable domain comprises SEQ ID NO: 10. In some embodiments, the heavy chain variable domain comprises SEQ ID NO: 11 and the light chain variable domain comprises SEQ ID NO: 12. In some embodiments, the heavy chain variable domain comprises SEQ ID NO: 17 and the light chain variable domain comprises SEQ ID NO: 18. In some embodiments, the heavy chain variable domain comprises SEQ ID NO: 19 and the light chain variable domain comprises SEQ ID NO: 20. In some embodiments, the heavy chain variable domain comprises SEQ ID NO: 21 and the light chain variable domain comprises SEQ ID NO: 22. In some embodiments, the heavy chain variable domain comprises SEQ ID NO: 25 and the light chain variable domain comprises SEQ ID NO: 26. In some embodiments, the heavy chain variable domain comprises SEQ ID NO: 29 and the light chain variable domain comprises SEQ ID NO: 30. In some embodiments, the heavy chain variable domain comprises SEQ ID NO: 31 and the light chain variable domain comprises SEQ ID NO: 32. In some embodiments, the heavy chain variable domain comprises SEQ ID NO: 33 and the light chain variable domain comprises SEQ ID NO: 34. In some embodiments, the heavy chain variable domain comprises SEQ ID NO: 37 and the light chain variable domain comprises SEQ ID NO: 38. In some embodiments, the heavy chain variable domain comprises SEQ ID NO: 39 and the light chain variable domain comprises SEQ ID NO: 40. In some embodiments, the heavy chain variable domain comprises SEQ ID NO: 43 and the light chain variable domain comprises SEQ ID NO: 44. In some embodiments, the heavy chain variable domain comprises SEQ ID NO: 45 and the light chain variable domain comprises SEQ ID NO: 46.

In some embodiments, the heavy chain variable domain comprises the CDR1, CDR2, and CDR3 regions of SEQ ID NO: 3; the CDR1, CDR2, and CDR3 regions of SEQ ID NO: 7; the CDR1, CDR2, and CDR3 regions of SEQ ID NO: 9; the CDR1, CDR2, and CDR3 regions of SEQ ID NO: 11; or the CDR1, CDR2, and CDR3 regions of SEQ ID NO: 17. In some embodiments, the heavy chain variable domain comprises the CDR1, CDR2, and CDR3 regions of SEQ ID NO: 19; the CDR1, CDR2, and CDR3 regions of SEQ ID NO: 21; the CDR1, CDR2, and CDR3 regions of SEQ ID NO: 25; the CDR1, CDR2, and CDR3 regions of SEQ ID NO: 29; or the CDR1, CDR2, and CDR3 regions of SEQ ID NO: 31. In some embodiments, the heavy chain variable domain comprises the CDR1, CDR2, and CDR3 regions of SEQ ID NO: 33; the CDR1, CDR2, and CDR3 regions of SEQ ID NO: 37; the CDR1, CDR2, and CDR3 regions of SEQ ID NO: 39; the CDR1, CDR2, and CDR3 regions of SEQ ID NO: 43; or the CDR1, CDR2, and CDR3 regions of SEQ ID NO: 45. In some embodiments, the light chain variable domain comprises the CDR1, CDR2, and CDR3 regions of SEQ ID NO: 4; the CDR1, CDR2, and CDR3 regions of SEQ ID NO: 8; the CDR1, CDR2, and CDR3 regions of SEQ ID NO: 10; the CDR1, CDR2, and CDR3 regions of SEQ ID NO: 12; or the CDR1, CDR2, and CDR3 regions of SEQ ID NO: 18. In some embodiments, the light chain variable domain comprises the CDR1, CDR2, and CDR3 regions of SEQ ID NO: 20; the CDR1, CDR2, and CDR3 regions of SEQ ID NO: 22; the CDR1, CDR2, and CDR3 regions of SEQ ID NO: 26; the CDR1, CDR2, and CDR3 regions of SEQ ID NO: 30; or the CDR1, CDR2, and CDR3 regions of SEQ ID NO: 32. In some embodiments, the light chain variable domain comprises the CDR1, CDR2, and CDR3 regions of SEQ ID NO: 34; the CDR1, CDR2, and CDR3 regions of SEQ ID NO: 38; the CDR1, CDR2, and CDR3 regions of SEQ ID NO: 40; the CDR1, CDR2, and CDR3 regions of SEQ ID NO: 44; or the CDR1, CDR2, and CDR3 regions of SEQ ID NO: 46.

In some embodiments, the antibody neutralizes a coronavirus. In some embodiments, the coronavirus is a SARS-CoV-2 virus. In some embodiments, the coronavirus is a SARS-CoV-2 variant strain. In some embodiments, the variant strain is selected from the group consisting of WA1, B.1.1.7, B1.526, B1.351, P1, B1.352, and B.1.427.

In some embodiments, the antibody specifically binds an epitope on the surface of the coronavirus. In some embodiments, the epitope comprises at least a portion of a coronavirus spike protein. In some embodiments, the at least a portion of a coronavirus spike protein comprises a receptor binding domain (RBD) of a spike protein. In some embodiments, the at least a portion of a coronavirus spike protein comprises a N-Terminal domain (NTD) of a spike protein. In some embodiments, the coronavirus is a SARS-CoV-2 coronavirus. In some embodiments, the composition is administered in combination with a second therapeutic agent.

In certain aspects, the invention provides a bispecific molecule comprising a first polypeptide chain and a second polypeptide chain, wherein: the first polypeptide chain comprises: a first light chain comprising a variable domain and a constant domain; a first heavy chain comprising a variable domain and a constant domain; the second polypeptide chain comprises: a second light chain comprising a variable domain and a constant domain; and a second heavy chain comprising a variable domain and a constant domain.

In some embodiments, the first polypeptide chain and the second polypeptide chain are covalently bonded to one another. In some embodiments, the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 51, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 55, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 67, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 71, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 75, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 79, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 83, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 87, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 91, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 95, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 99, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 103, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 107, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 115, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 119, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 123, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 127, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 131, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 139, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 143, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 147, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 151, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 155, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 159, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 163, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 167, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 171, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 175, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 179, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO:183.

In some embodiments, the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 52, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 56, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 68; a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 72, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 76, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 80, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 84, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 88, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 92, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 96, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 100, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 104, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 108, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 116, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 120, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 124, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 128, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 132, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 140, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 144, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 148, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 152, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 156, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 160, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 164, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 168, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 172, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 176, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 180, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO:184.

In some embodiments, the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 53, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 57, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 69, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 73, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 77, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 81, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 85, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 89, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 93, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 97, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 101, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 105, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 109, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 117, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 121, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 125, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 129, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 133, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 141, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 145, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 149, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 153, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 157, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 161, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 165, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 169, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 173, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 177, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 181, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 185.

In some embodiments, the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 54, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 58, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 70, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 74, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 78, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 82, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 86, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 90, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 94, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 98, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 102, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 106, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 110, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 118, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 122, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 126, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 130, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 134, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 142, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 146, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 150, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 154, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 158, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 162, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 166, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 170, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 174, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 178, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 182, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 186.

In some embodiments, variable domain of the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 41.

In some embodiments, variable domain of the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 42.

In some embodiments, variable domain of the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 29, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 23, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 39, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9.

In some embodiments, variable domain of the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 30, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 24, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 40, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10.

In some embodiments, the variable domain of the first heavy chain comprises the CDR1, CDR2, and CDR3 of SEQ ID NO: 51; the CDR1, CDR2, and CDR3 of SEQ ID NO: 55; the CDR1, CDR2, and CDR3 of SEQ ID NO: 67; the CDR1, CDR2, and CDR3 of SEQ ID NO: 71; the CDR1, CDR2, and CDR3 of SEQ ID NO: 75; the CDR1, CDR2, and CDR3 of SEQ ID NO: 79; the CDR1, CDR2, and CDR3 of SEQ ID NO: 83; the CDR1, CDR2, and CDR3 of SEQ ID NO: 87; the CDR1, CDR2, and CDR3 of SEQ ID NO: 91; the CDR1, CDR2, and CDR3 of SEQ ID NO: 95; the CDR1, CDR2, and CDR3 of SEQ ID NO: 99; the CDR1, CDR2, and CDR3 of SEQ ID NO: 103; the CDR1, CDR2, and CDR3 of SEQ ID NO: 107; the CDR1, CDR2, and CDR3 of SEQ ID NO: 115; the CDR1, CDR2, and CDR3 of SEQ ID NO: 119, the CDR1, CDR2, and CDR3 of SEQ ID NO: 123, the CDR1, CDR2, and CDR3 of SEQ ID NO: 127; the CDR1, CDR2, and CDR3 of SEQ ID NO: 131; the CDR1, CDR2, and CDR3 of SEQ ID NO: 139; the CDR1, CDR2, and CDR3 of SEQ ID NO: 143; the CDR1, CDR2, and CDR3 of SEQ ID NO: 147; the CDR1, CDR2, and CDR3 of SEQ ID NO: 151; the CDR1, CDR2, and CDR3 of SEQ ID NO: 155; the CDR1, CDR2, and CDR3 of SEQ ID NO: 159; the CDR1, CDR2, and CDR3 of SEQ ID NO: 163; the CDR1, CDR2, and CDR3 of SEQ ID NO: 167, the CDR1, CDR2, and CDR3 of SEQ ID NO: 171, the CDR1, CDR2, and CDR3 of SEQ ID NO: 175, the CDR1, CDR2, and CDR3 of SEQ ID NO: 179, or the CDR1, CDR2, and CDR3 of SEQ ID NO: 183.

In some embodiments, the variable domain of the first light chain comprises the CDR1, CDR2, and CDR3 of SEQ ID NO: 52; the CDR1, CDR2, and CDR3 of SEQ ID NO: 56; the CDR1, CDR2, and CDR3 of SEQ ID NO: 68; the CDR1, CDR2, and CDR3 of SEQ ID NO: 72; the CDR1, CDR2, and CDR3 of SEQ ID NO: 76; the CDR1, CDR2, and CDR3 of SEQ ID NO: 80; the CDR1, CDR2, and CDR3 of SEQ ID NO: 84; the CDR1, CDR2, and CDR3 of SEQ ID NO: 88; the CDR1, CDR2, and CDR3 of SEQ ID NO: 92; the CDR1, CDR2, and CDR3 of SEQ ID NO: 96; the CDR1, CDR2, and CDR3 of SEQ ID NO: 100; the CDR1, CDR2, and CDR3 of SEQ ID NO: 104; the CDR1, CDR2, and CDR3 of SEQ ID NO: 108; the CDR1, CDR2, and CDR3 of SEQ ID NO: 116; the CDR1, CDR2, and CDR3 of SEQ ID NO: 120; the CDR1, CDR2, and CDR3 of SEQ ID NO: 124; the CDR1, CDR2, and CDR3 of SEQ ID NO: 128; the CDR1, CDR2, and CDR3 of SEQ ID NO: 132; the CDR1, CDR2, and CDR3 of SEQ ID NO: 140; the CDR1, CDR2, and CDR3 of SEQ ID NO: 144; the CDR1, CDR2, and CDR3 of SEQ ID NO: 148; the CDR1, CDR2, and CDR3 of SEQ ID NO: 152; the CDR1, CDR2, and CDR3 of SEQ ID NO: 156; the CDR1, CDR2, and CDR3 of SEQ ID NO: 160; the CDR1, CDR2, and CDR3 of SEQ ID NO: 164; the CDR1, CDR2, and CDR3 of SEQ ID NO: 168, the CDR1, CDR2, and CDR3 of SEQ ID NO: 172, the CDR1, CDR2, and CDR3 of SEQ ID NO: 176, the CDR1, CDR2, and CDR3 of SEQ ID NO: 180, or the CDR1, CDR2, and CDR3 of SEQ ID NO: 184.

In some embodiments, the variable domain of the second heavy chain comprises the CDR1, CDR2, and CDR3 of SEQ ID NO: 53; the CDR1, CDR2, and CDR3 of SEQ ID NO: 57; the CDR1, CDR2, and CDR3 of SEQ ID NO: 69; the CDR1, CDR2, and CDR3 of SEQ ID NO: 73; the CDR1, CDR2, and CDR3 of SEQ ID NO: 77; the CDR1, CDR2, and CDR3 of SEQ ID NO: 81; the CDR1, CDR2, and CDR3 of SEQ ID NO: 85; the CDR1, CDR2, and CDR3 of SEQ ID NO: 89; the CDR1, CDR2, and CDR3 of SEQ ID NO: 93; the CDR1, CDR2, and CDR3 of SEQ ID NO: 97; the CDR1, CDR2, and CDR3 of SEQ ID NO: 101; the CDR1, CDR2, and CDR3 of SEQ ID NO: 105; the CDR1, CDR2, and CDR3 of SEQ ID NO: 109; the CDR1, CDR2, and CDR3 of SEQ ID NO: 117; the CDR1, CDR2, and CDR3 of SEQ ID NO: 121; the CDR1, CDR2, and CDR3 of SEQ ID NO: 125; the CDR1, CDR2, and CDR3 of SEQ ID NO: 129; the CDR1, CDR2, and CDR3 of SEQ ID NO: 133; the CDR1, CDR2, and CDR3 of SEQ ID NO: 141; the CDR1, CDR2, and CDR3 of SEQ ID NO: 145; the CDR1, CDR2, and CDR3 of SEQ ID NO: 149; the CDR1, CDR2, and CDR3 of SEQ ID NO: 153; the CDR1, CDR2, and CDR3 of SEQ ID NO: 157; the CDR1, CDR2, and CDR3 of SEQ ID NO: 161; the CDR1, CDR2, and CDR3 of SEQ ID NO: 165; the CDR1, CDR2, and CDR3 of SEQ ID NO: 169, the CDR1, CDR2, and CDR3 of SEQ ID NO: 173, the CDR1, CDR2, and CDR3 of SEQ ID NO: 177, the CDR1, CDR2, and CDR3 of SEQ ID NO: 181, or the CDR1, CDR2, and CDR3 of SEQ ID NO: 185.

In some embodiments, the variable domain of the second light chain comprises the CDR1, CDR2, and CDR3 of SEQ ID NO: 54; the CDR1, CDR2, and CDR3 of SEQ ID NO: 58; the CDR1, CDR2, and CDR3 of SEQ ID NO: 70; the CDR1, CDR2, and CDR3 of SEQ ID NO: 74; the CDR1, CDR2, and CDR3 of SEQ ID NO: 78; the CDR1, CDR2, and CDR3 of SEQ ID NO: 82; the CDR1, CDR2, and CDR3 of SEQ ID NO: 86; the CDR1, CDR2, and CDR3 of SEQ ID NO: 90; the CDR1, CDR2, and CDR3 of SEQ ID NO: 94; the CDR1, CDR2, and CDR3 of SEQ ID NO: 98; the CDR1, CDR2, and CDR3 of SEQ ID NO: 102; the CDR1, CDR2, and CDR3 of SEQ ID NO: 106; the CDR1, CDR2, and CDR3 of SEQ ID NO: 110; the CDR1, CDR2, and CDR3 of SEQ ID NO: 118; the CDR1, CDR2, and CDR3 of SEQ ID NO: 122; the CDR1, CDR2, and CDR3 of SEQ ID NO: 126; the CDR1, CDR2, and CDR3 of SEQ ID NO: 130; the CDR1, CDR2, and CDR3 of SEQ ID NO: 134; the CDR1, CDR2, and CDR3 of SEQ ID NO: 142; the CDR1, CDR2, and CDR3 of SEQ ID NO: 146; the CDR1, CDR2, and CDR3 of SEQ ID NO: 150; the CDR1, CDR2, and CDR3 of SEQ ID NO: 154; the CDR1, CDR2, and CDR3 of SEQ ID NO: 158; the CDR1, CDR2, and CDR3 of SEQ ID NO: 162, the CDR1, CDR2, and CDR3 of SEQ ID NO: 166; the CDR1, CDR2, and CDR3 of SEQ ID NO: 170, the CDR1, CDR2, and CDR3 of SEQ ID NO: 174, the CDR1, CDR2, and CDR3 of SEQ ID NO: 178, the CDR1, CDR2, and CDR3 of SEQ ID NO: 182, or the CDR1, CDR2, and CDR3 of SEQ ID NO: 186.

In some embodiments, the molecule comprises SEQ ID NO: 51, SEQ ID NO: 52, SEQ ID NO: 53, and SEQ ID NO: 54. In some embodiments, the molecule comprises SEQ ID NO: 55, SEQ ID NO: 56, SEQ ID NO: 57, and SEQ ID NO: 58. In some embodiments, the molecule comprises SEQ ID NO: 67, SEQ ID NO: 68, SEQ ID NO: 69, and SEQ ID NO: 70. In some embodiments, the molecule comprises SEQ ID NO: 71, SEQ ID NO: 72, SEQ ID NO: 73, and SEQ ID NO: 74. In some embodiments, the molecule comprises SEQ ID NO: 75, SEQ ID NO: 76, SEQ ID NO: 77, and SEQ ID NO: 78. In some embodiments, the molecule comprises SEQ ID NO: 79, SEQ ID NO: 80, SEQ ID NO: 81, and SEQ ID NO: 82. In some embodiments, the molecule comprises SEQ ID NO: 83, SEQ ID NO: 84, SEQ ID NO: 85, and SEQ ID NO: 86. In some embodiments, the molecule comprises SEQ ID NO: 87, SEQ ID NO: 88, SEQ ID NO: 89, and SEQ ID NO: 90. In some embodiments, the molecule comprises SEQ ID NO: 91, SEQ ID NO: 92, SEQ ID NO: 93, and SEQ ID NO: 94. In some embodiments, the molecule comprises SEQ ID NO: 95, SEQ ID NO: 96, SEQ ID NO: 97, and SEQ ID NO: 98. In some embodiments, the molecule comprises SEQ ID NO: 99, SEQ ID NO: 100, SEQ ID NO: 101, and SEQ ID NO: 102. In some embodiments, the molecule comprises SEQ ID NO: 103, SEQ ID NO: 104, SEQ ID NO: 105, and SEQ ID NO: 106. In some embodiments, the molecule comprises SEQ ID NO: 107, SEQ ID NO: 108, SEQ ID NO: 109, and SEQ ID NO: 110. In some embodiments, the molecule comprises SEQ ID NO: 115, SEQ ID NO: 116, SEQ ID NO: 117, and SEQ ID NO: 118. In some embodiments, the molecule comprises SEQ ID NO: 119, SEQ ID NO: 120, SEQ ID NO: 121, and SEQ ID NO: 122.

In some embodiments, the molecule comprises SEQ ID NO: 123, SEQ ID NO: 124, SEQ ID NO: 125, and SEQ ID NO: 126. In some embodiments, the molecule comprises SEQ ID NO: 127, SEQ ID NO: 128, SEQ ID NO: 129, and SEQ ID NO: 130. In some embodiments, the molecule comprises SEQ ID NO: 131, SEQ ID NO: 132, SEQ ID NO: 133, and SEQ ID NO: 133. In some embodiments, the molecule comprises SEQ ID NO: 139, SEQ ID NO: 140, SEQ ID NO: 141, and SEQ ID NO: 142. In some embodiments, the molecule comprises SEQ ID NO: 143, SEQ ID NO: 144, SEQ ID NO: 145, and SEQ ID NO: 146. In some embodiments, the molecule comprises SEQ ID NO: 147, SEQ ID NO: 148, SEQ ID NO: 149, and SEQ ID NO: 150. In some embodiments, the molecule comprises SEQ ID NO: 151, SEQ ID NO: 152, SEQ ID NO: 153, and SEQ ID NO: 154. In some embodiments, the molecule comprises SEQ ID NO: 155, SEQ ID NO: 156, SEQ ID NO: 157, and SEQ ID NO: 158. In some embodiments, the molecule comprises SEQ ID NO: 159, SEQ ID NO: 160, SEQ ID NO: 161, and SEQ ID NO: 162. In some embodiments, the molecule comprises SEQ ID NO: 163, SEQ ID NO: 164, SEQ ID NO: 165, and SEQ ID NO: 166. In some embodiments, the molecule comprises SEQ ID NO: 167, SEQ ID NO: 168, SEQ ID NO: 169, and SEQ ID NO: 170. In some embodiments, the molecule comprises SEQ ID NO: 171, SEQ ID NO: 172, SEQ ID NO: 173, and SEQ ID NO: 174. In some embodiments, the molecule comprises SEQ ID NO: 175, SEQ ID NO: 176, SEQ ID NO: 177, and SEQ ID NO: 178. In some embodiments, the molecule comprises SEQ ID NO: 179, SEQ ID NO: 180, SEQ ID NO: 181, and SEQ ID NO: 182. In some embodiments, the molecule comprises SEQ ID NO: 183, SEQ ID NO: 184, SEQ ID NO: 185, and SEQ ID NO: 186.

In some embodiments, the molecule further comprises one or more disulfide bonds. In some embodiments, the molecule further comprises one or more linker sequences. In some embodiments, the molecule is capable of neutralizing a coronavirus. In some embodiments, the coronavirus is a SARS-CoV-2 virus. In some embodiments, the SARS-CoV-2 virus strain is selected from the group consisting of WA1, B.1.1.7, B1.526, B1.351, P1, B1.352, and B.1.427.

In some embodiments, the molecule specifically recognizes a first epitope and a second epitope on a SARS-CoV-2 particle. In some embodiments, the first epitope comprises at least a portion of a receptor binding domain (RBD) on a spike protein of a SARS-CoV-2 particle. In some embodiments, the second epitope comprises at least a portion of a N-terminal domain (NTD) on a spike protein of a SARS-CoV-2 particle.

In certain aspects, the invention provides a method of treating or preventing a COVID-19 infection in a subject in need thereof, the method comprising administering to the subject a composition comprising a bispecific molecule comprising a first polypeptide chain and a second polypeptide chain, wherein: the first polypeptide chain comprises: a first light chain comprising a variable domain and a constant domain; a first heavy chain comprising a variable domain and a constant domain; the second polypeptide chain comprises: a second light chain comprising a variable domain and a constant domain; and a second heavy chain comprising a variable domain and a constant domain.

In some embodiments, the first polypeptide chain and the second polypeptide chain are covalently bonded to one another. In some embodiments, the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 51, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 55, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 67, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 71, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 75, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 79, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 83, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 87, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 91, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 95, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 99, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 103, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 107, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 115, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 119, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 123, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 127, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 131, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 139, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 143, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 147, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 151, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 155, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 159, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 163, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 167, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 171, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 175, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 179, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO:183.

In some embodiments, the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 52, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 56, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 68; a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 72, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 76, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 80, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 84, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 88, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 92, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 96, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 100, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 104, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 108, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 116, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 120, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 124, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 128, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 132, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 140, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 144, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 148, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 152, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 156, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 160, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 164, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 168, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 172, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 176, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 180, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO:184.

In some embodiments, the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 53, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 57, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 69, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 73, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 77, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 81, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 85, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 89, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 93, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 97, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 101, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 105, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 109, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 117, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 121, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 125, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 129, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 133, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 141, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 145, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 149, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 153, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 157, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 161, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 165, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 169, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 173, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 177, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 181, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 185.

In some embodiments, the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 54, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 58, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 70, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 74, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 78, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 82, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 86, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 90, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 94, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 98, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 102, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 106, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 110, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 118, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 122, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 126, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 130, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 134, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 142, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 146, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 150, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 154, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 158, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 162, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 166, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 170, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 174, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 178, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 182, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 186.

In some embodiments, the variable domain of the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 41.

In some embodiments, the variable domain of the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 42.

In some embodiments, the variable domain of the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 29, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 23, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 39, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9.

In some embodiments, variable domain of the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 30, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 24, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 40, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10.

In some embodiments, the variable domain of the first heavy chain comprises the CDR1, CDR2, and CDR3 of SEQ ID NO: 51; the CDR1, CDR2, and CDR3 of SEQ ID NO: 55; the CDR1, CDR2, and CDR3 of SEQ ID NO: 67; the CDR1, CDR2, and CDR3 of SEQ ID NO: 71; the CDR1, CDR2, and CDR3 of SEQ ID NO: 75; the CDR1, CDR2, and CDR3 of SEQ ID NO: 79; the CDR1, CDR2, and CDR3 of SEQ ID NO: 83; the CDR1, CDR2, and CDR3 of SEQ ID NO: 87; the CDR1, CDR2, and CDR3 of SEQ ID NO: 91; the CDR1, CDR2, and CDR3 of SEQ ID NO: 95; the CDR1, CDR2, and CDR3 of SEQ ID NO: 99; the CDR1, CDR2, and CDR3 of SEQ ID NO: 103; the CDR1, CDR2, and CDR3 of SEQ ID NO: 107; the CDR1, CDR2, and CDR3 of SEQ ID NO: 115; the CDR1, CDR2, and CDR3 of SEQ ID NO: 119, the CDR1, CDR2, and CDR3 of SEQ ID NO: 123, the CDR1, CDR2, and CDR3 of SEQ ID NO: 127; the CDR1, CDR2, and CDR3 of SEQ ID NO: 131; the CDR1, CDR2, and CDR3 of SEQ ID NO: 139; the CDR1, CDR2, and CDR3 of SEQ ID NO: 143; the CDR1, CDR2, and CDR3 of SEQ ID NO: 147; the CDR1, CDR2, and CDR3 of SEQ ID NO: 151; the CDR1, CDR2, and CDR3 of SEQ ID NO: 155; the CDR1, CDR2, and CDR3 of SEQ ID NO: 159; the CDR1, CDR2, and CDR3 of SEQ ID NO: 163; the CDR1, CDR2, and CDR3 of SEQ ID NO: 167, the CDR1, CDR2, and CDR3 of SEQ ID NO: 171, the CDR1, CDR2, and CDR3 of SEQ ID NO: 175, the CDR1, CDR2, and CDR3 of SEQ ID NO: 179, or the CDR1, CDR2, and CDR3 of SEQ ID NO: 183.

In some embodiments, the variable domain of the first light chain comprises the CDR1, CDR2, and CDR3 of SEQ ID NO: 52; the CDR1, CDR2, and CDR3 of SEQ ID NO: 56; the CDR1, CDR2, and CDR3 of SEQ ID NO: 68; the CDR1, CDR2, and CDR3 of SEQ ID NO: 72; the CDR1, CDR2, and CDR3 of SEQ ID NO: 76; the CDR1, CDR2, and CDR3 of SEQ ID NO: 80; the CDR1, CDR2, and CDR3 of SEQ ID NO: 84; the CDR1, CDR2, and CDR3 of SEQ ID NO: 88; the CDR1, CDR2, and CDR3 of SEQ ID NO: 92; the CDR1, CDR2, and CDR3 of SEQ ID NO: 96; the CDR1, CDR2, and CDR3 of SEQ ID NO: 100; the CDR1, CDR2, and CDR3 of SEQ ID NO: 104; the CDR1, CDR2, and CDR3 of SEQ ID NO: 108; the CDR1, CDR2, and CDR3 of SEQ ID NO: 116; the CDR1, CDR2, and CDR3 of SEQ ID NO: 120; the CDR1, CDR2, and CDR3 of SEQ ID NO: 124; the CDR1, CDR2, and CDR3 of SEQ ID NO: 128; the CDR1, CDR2, and CDR3 of SEQ ID NO: 132; the CDR1, CDR2, and CDR3 of SEQ ID NO: 140; the CDR1, CDR2, and CDR3 of SEQ ID NO: 144; the CDR1, CDR2, and CDR3 of SEQ ID NO: 148; the CDR1, CDR2, and CDR3 of SEQ ID NO: 152; the CDR1, CDR2, and CDR3 of SEQ ID NO: 156; the CDR1, CDR2, and CDR3 of SEQ ID NO: 160; the CDR1, CDR2, and CDR3 of SEQ ID NO: 164; the CDR1, CDR2, and CDR3 of SEQ ID NO: 168, the CDR1, CDR2, and CDR3 of SEQ ID NO: 172, the CDR1, CDR2, and CDR3 of SEQ ID NO: 176, the CDR1, CDR2, and CDR3 of SEQ ID NO: 180, or the CDR1, CDR2, and CDR3 of SEQ ID NO: 184.

In some embodiments, the variable domain of the second heavy chain comprises the CDR1, CDR2, and CDR3 of SEQ ID NO: 53; the CDR1, CDR2, and CDR3 of SEQ ID NO: 57; the CDR1, CDR2, and CDR3 of SEQ ID NO: 69; the CDR1, CDR2, and CDR3 of SEQ ID NO: 73; the CDR1, CDR2, and CDR3 of SEQ ID NO: 77; the CDR1, CDR2, and CDR3 of SEQ ID NO: 81; the CDR1, CDR2, and CDR3 of SEQ ID NO: 85; the CDR1, CDR2, and CDR3 of SEQ ID NO: 89; the CDR1, CDR2, and CDR3 of SEQ ID NO: 93; the CDR1, CDR2, and CDR3 of SEQ ID NO: 97; the CDR1, CDR2, and CDR3 of SEQ ID NO: 101; the CDR1, CDR2, and CDR3 of SEQ ID NO: 105; the CDR1, CDR2, and CDR3 of SEQ ID NO: 109; the CDR1, CDR2, and CDR3 of SEQ ID NO: 117; the CDR1, CDR2, and CDR3 of SEQ ID NO: 121; the CDR1, CDR2, and CDR3 of SEQ ID NO: 125; the CDR1, CDR2, and CDR3 of SEQ ID NO: 129; the CDR1, CDR2, and CDR3 of SEQ ID NO: 133; the CDR1, CDR2, and CDR3 of SEQ ID NO: 141; the CDR1, CDR2, and CDR3 of SEQ ID NO: 145; the CDR1, CDR2, and CDR3 of SEQ ID NO: 149; the CDR1, CDR2, and CDR3 of SEQ ID NO: 153; the CDR1, CDR2, and CDR3 of SEQ ID NO: 157; the CDR1, CDR2, and CDR3 of SEQ ID NO: 161; the CDR1, CDR2, and CDR3 of SEQ ID NO: 165; the CDR1, CDR2, and CDR3 of SEQ ID NO: 169, the CDR1, CDR2, and CDR3 of SEQ ID NO: 173, the CDR1, CDR2, and CDR3 of SEQ ID NO: 177, the CDR1, CDR2, and CDR3 of SEQ ID NO: 181, or the CDR1, CDR2, and CDR3 of SEQ ID NO: 185.

In some embodiments, the variable domain of the second light chain comprises the CDR1, CDR2, and CDR3 of SEQ ID NO: 54; the CDR1, CDR2, and CDR3 of SEQ ID NO: 58; the CDR1, CDR2, and CDR3 of SEQ ID NO: 70; the CDR1, CDR2, and CDR3 of SEQ ID NO: 74; the CDR1, CDR2, and CDR3 of SEQ ID NO: 78; the CDR1, CDR2, and CDR3 of SEQ ID NO: 82; the CDR1, CDR2, and CDR3 of SEQ ID NO: 86; the CDR1, CDR2, and CDR3 of SEQ ID NO: 90; the CDR1, CDR2, and CDR3 of SEQ ID NO: 94; the CDR1, CDR2, and CDR3 of SEQ ID NO: 98; the CDR1, CDR2, and CDR3 of SEQ ID NO: 102; the CDR1, CDR2, and CDR3 of SEQ ID NO: 106; the CDR1, CDR2, and CDR3 of SEQ ID NO: 110; the CDR1, CDR2, and CDR3 of SEQ ID NO: 118; the CDR1, CDR2, and CDR3 of SEQ ID NO: 122; the CDR1, CDR2, and CDR3 of SEQ ID NO: 126; the CDR1, CDR2, and CDR3 of SEQ ID NO: 130; the CDR1, CDR2, and CDR3 of SEQ ID NO: 134; the CDR1, CDR2, and CDR3 of SEQ ID NO: 142; the CDR1, CDR2, and CDR3 of SEQ ID NO: 146; the CDR1, CDR2, and CDR3 of SEQ ID NO: 150; the CDR1, CDR2, and CDR3 of SEQ ID NO: 154; the CDR1, CDR2, and CDR3 of SEQ ID NO: 158; the CDR1, CDR2, and CDR3 of SEQ ID NO: 162, the CDR1, CDR2, and CDR3 of SEQ ID NO: 166; the CDR1, CDR2, and CDR3 of SEQ ID NO: 170, the CDR1, CDR2, and CDR3 of SEQ ID NO: 174, the CDR1, CDR2, and CDR3 of SEQ ID NO: 178, the CDR1, CDR2, and CDR3 of SEQ ID NO: 182, or the CDR1, CDR2, and CDR3 of SEQ ID NO: 186.

In some embodiments, the molecule comprises SEQ ID NO: 51, SEQ ID NO: 52, SEQ ID NO: 53, and SEQ ID NO: 54. In some embodiments, the molecule comprises SEQ ID NO: 55, SEQ ID NO: 56, SEQ ID NO: 57, and SEQ ID NO: 58. In some embodiments, the molecule comprises SEQ ID NO: 67, SEQ ID NO: 68, SEQ ID NO: 69, and SEQ ID NO: 70. In some embodiments, the molecule comprises SEQ ID NO: 71, SEQ ID NO: 72, SEQ ID NO: 73, and SEQ ID NO: 74. In some embodiments, the molecule comprises SEQ ID NO: 75, SEQ ID NO: 76, SEQ ID NO: 77, and SEQ ID NO: 78. In some embodiments, the molecule comprises SEQ ID NO: 79, SEQ ID NO: 80, SEQ ID NO: 81, and SEQ ID NO: 82. In some embodiments, the molecule comprises SEQ ID NO: 83, SEQ ID NO: 84, SEQ ID NO: 85, and SEQ ID NO: 86. In some embodiments, the molecule comprises SEQ ID NO: 87, SEQ ID NO: 88, SEQ ID NO: 89, and SEQ ID NO: 90. In some embodiments, the molecule comprises SEQ ID NO: 91, SEQ ID NO: 92, SEQ ID NO: 93, and SEQ ID NO: 94. In some embodiments, the molecule comprises SEQ ID NO: 95, SEQ ID NO: 96, SEQ ID NO: 97, and SEQ ID NO: 98. In some embodiments, the molecule comprises SEQ ID NO: 99, SEQ ID NO: 100, SEQ ID NO: 101, and SEQ ID NO: 102. In some embodiments, the molecule comprises SEQ ID NO: 103, SEQ ID NO: 104, SEQ ID NO: 105, and SEQ ID NO: 106. In some embodiments, the molecule comprises SEQ ID NO: 107, SEQ ID NO: 108, SEQ ID NO: 109, and SEQ ID NO: 110. In some embodiments, the molecule comprises SEQ ID NO: 115, SEQ ID NO: 116, SEQ ID NO: 117, and SEQ ID NO: 118.

In some embodiments, the molecule comprises SEQ ID NO: 119, SEQ ID NO: 120, SEQ ID NO: 121, and SEQ ID NO: 122. In some embodiments, the molecule comprises SEQ ID NO: 123, SEQ ID NO: 124, SEQ ID NO: 125, and SEQ ID NO: 126. In some embodiments, the molecule comprises SEQ ID NO: 127, SEQ ID NO: 128, SEQ ID NO: 129, and SEQ ID NO: 130. In some embodiments, the molecule comprises SEQ ID NO: 131, SEQ ID NO: 132, SEQ ID NO: 133, and SEQ ID NO: 133. In some embodiments, the molecule comprises SEQ ID NO: 139, SEQ ID NO: 140, SEQ ID NO: 141, and SEQ ID NO: 142. In some embodiments, the molecule comprises SEQ ID NO: 143, SEQ ID NO: 144, SEQ ID NO: 145, and SEQ ID NO: 146. In some embodiments, the molecule comprises SEQ ID NO: 147, SEQ ID NO: 148, SEQ ID NO: 149, and SEQ ID NO: 150. In some embodiments, the molecule comprises SEQ ID NO: 151, SEQ ID NO: 152, SEQ ID NO: 153, and SEQ ID NO: 154. In some embodiments, the molecule comprises SEQ ID NO: 155, SEQ ID NO: 156, SEQ ID NO: 157, and SEQ ID NO: 158. In some embodiments, the molecule comprises SEQ ID NO: 159, SEQ ID NO: 160, SEQ ID NO: 161, and SEQ ID NO: 162. In some embodiments, the molecule comprises SEQ ID NO: 163, SEQ ID NO: 164, SEQ ID NO: 165, and SEQ ID NO: 166. In some embodiments, the molecule comprises SEQ ID NO: 167, SEQ ID NO: 168, SEQ ID NO: 169, and SEQ ID NO: 170. In some embodiments, the molecule comprises SEQ ID NO: 171, SEQ ID NO: 172, SEQ ID NO: 173, and SEQ ID NO: 174. In some embodiments, the molecule comprises SEQ ID NO: 175, SEQ ID NO: 176, SEQ ID NO: 177, and SEQ ID NO: 178. In some embodiments, the molecule comprises SEQ ID NO: 179, SEQ ID NO: 180, SEQ ID NO: 181, and SEQ ID NO: 182. In some embodiments, the molecule comprises SEQ ID NO: 183, SEQ ID NO: 184, SEQ ID NO: 185, and SEQ ID NO: 186.

In some embodiments, the molecule further comprises one or more disulfide bonds. In some embodiments, the molecule further comprises one or more linker sequences. In some embodiments, the molecule is capable of neutralizing a coronavirus. In some embodiments, the coronavirus is a SARS-CoV-2 virus. In some embodiments, the SARS-CoV-2 virus strain is selected from the group consisting of WA1, B.1.1.7, B1.526, B1.351, P1, B1.352, and B.1.427. In some embodiments, the molecule specifically recognizes a first epitope and a second epitope on a SARS-CoV-2 particle. In some embodiments, the first epitope comprises at least a portion of a receptor binding domain (RBD) on a spike protein of a SARS-CoV-2 particle. In some embodiments, the second epitope comprises at least a portion of a N-terminal domain (NTD) on a spike protein of a SARS-CoV-2 particle. In some embodiments, the composition is administered in combination with a second therapeutic agent.

In certain aspects, the invention provides a synthesized monoclonal antibody, or a functional fragment thereof, comprising a heavy chain having a variable domain and a constant domain and a light chain having a variable domain and a constant domain, wherein the antibody binds an antigen on a coronavirus particle.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 41. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 23. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 42. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 2.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 24. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 41 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 42. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 2. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 23 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 24. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28.

In some embodiments, the heavy chain variable domain comprises SEQ ID NO: 5 and the light chain variable domain comprises SEQ ID NO: 6. In some embodiments, the heavy chain variable domain comprises SEQ ID NO: 41 and the light chain variable domain comprises SEQ ID NO: 42. In some embodiments, the heavy chain variable domain comprises SEQ ID NO: 35 and the light chain variable domain comprises SEQ ID NO: 36. In some embodiments, the heavy chain variable domain comprises SEQ ID NO: 1 and the light chain variable domain comprises SEQ ID NO: 2. In some embodiments, the heavy chain variable domain comprises SEQ ID NO: 23 and the light chain variable domain comprises SEQ ID NO: 24. In some embodiments, the heavy chain variable domain comprises SEQ ID NO: 13 and the light chain variable domain comprises SEQ ID NO: 14. In some embodiments, the heavy chain variable domain comprises SEQ ID NO: 27 and the light chain variable domain comprises SEQ ID NO: 28. In some embodiments, the heavy chain variable domain comprises the CDR1, CDR2, and CDR3 regions of SEQ ID NO: 5. In some embodiments, the heavy chain variable domain comprises the CDR1, CDR2, and CDR3 regions of SEQ ID NO: 41. In some embodiments, the heavy chain variable domain comprises the CDR1, CDR2, and CDR3 regions of SEQ ID NO: 1.

In some embodiments, the heavy chain variable domain comprises the CDR1, CDR2, and CDR3 regions of SEQ ID NO: 23. In some embodiments, the heavy chain variable domain comprises the CDR1, CDR2, and CDR3 regions of SEQ ID NO: 13. In some embodiments, the heavy chain variable domain comprises the CDR1, CDR2, and CDR3 regions of SEQ ID NO: 27.

In some embodiments, the light chain variable domain comprises the CDR1, CDR2, and CDR3 regions of SEQ ID NO: 6. In some embodiments, the light chain variable domain comprises the CDR1, CDR2, and CDR3 regions of SEQ ID NO: 42. In some embodiments, the light chain variable domain comprises the CDR1, CDR2, and CDR3 regions of SEQ ID NO: 36. In some embodiments, the light chain variable domain comprises the CDR1, CDR2, and CDR3 regions of SEQ ID NO: 2. In some embodiments, the light chain variable domain comprises the CDR1, CDR2, and CDR3 regions of SEQ ID NO: 24. In some embodiments, the light chain variable domain comprises the CDR1, CDR2, and CDR3 regions of SEQ ID NO: 14. In some embodiments, the light chain variable domain comprises the CDR1, CDR2, and CDR3 regions of SEQ ID NO: 28. In some embodiments, the heavy chain variable domain comprises the CDR1, CDR2, and CDR3 regions of SEQ ID NO: 5 and the light chain variable domain comprises the CDR1, CDR2, and CDR3 regions of SEQ ID NO: 6. In some embodiments, the heavy chain variable domain comprises the CDR1, CDR2, and CDR3 regions of SEQ ID NO: 41 and the light chain variable domain comprises the CDR1, CDR2, and CDR3 regions of SEQ ID NO: 42. In some embodiments, the heavy chain variable domain comprises the CDR1, CDR2, and CDR3 regions of SEQ ID NO: 35 and the light chain variable domain comprises the CDR1, CDR2, and CDR3 regions of SEQ ID NO: 36. In some embodiments, the heavy chain variable domain comprises the CDR1, CDR2, and CDR3 regions of SEQ ID NO: 1 and the light chain variable domain comprises the CDR1, CDR2, and CDR3 regions of SEQ ID NO: 2.

In some embodiments, the heavy chain variable domain comprises the CDR1, CDR2, and CDR3 regions of SEQ ID NO: 23 and the light chain variable domain comprises the CDR1, CDR2, and CDR3 regions of SEQ ID NO: 24. In some embodiments, the heavy chain variable domain comprises the CDR1, CDR2, and CDR3 regions of SEQ ID NO: 13 and the light chain variable domain comprises the CDR1, CDR2, and CDR3 regions of SEQ ID NO: 14. In some embodiments, the heavy chain variable domain comprises the CDR1, CDR2, and CDR3 regions of SEQ ID NO: 27 and the light chain variable domain comprises the CDR1, CDR2, and CDR3 regions of SEQ ID NO: 28.

In some embodiments, the antibody neutralizes a coronavirus. In some embodiments, the coronavirus is a SARS-CoV-2 virus. In some embodiments, the coronavirus is a SARS-CoV-2 variant strain. In some embodiments, the variant strain is selected from the group consisting of WA1, B.1.1.7, B1.526, B1.351, P1, B1.352, and B.1.427. In some embodiments, the antibody specifically binds an epitope on the surface of the coronavirus. In some embodiments, the epitope comprises at least a portion of a coronavirus spike protein. In some embodiments, the at least a portion of a coronavirus spike protein comprises a receptor binding domain (RBD) of a spike protein. In some embodiments, the at least a portion of a coronavirus spike protein comprises a N-Terminal domain (NTD) of a spike protein. In some embodiments, the coronavirus is a SARS-CoV-2 coronavirus.

In certain aspects, the invention provides a method of treating or preventing a COVID-19 infection in a subject in need thereof, the method comprising administering to the subject a therapeutically effective amount of a synthesized monoclonal antibody, or a functional fragment thereof, comprising a heavy chain having a variable domain and a constant domain and a light chain having a variable domain and a constant domain, wherein the antibody binds an antigen on a coronavirus particle.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 41. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 23. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 42. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 2. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 24. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 41 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 42. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 2. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 23 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 24.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28.

In some embodiments, the heavy chain variable domain comprises SEQ ID NO: 5 and the light chain variable domain comprises SEQ ID NO: 6. In some embodiments, the heavy chain variable domain comprises SEQ ID NO: 41 and the light chain variable domain comprises SEQ ID NO: 42. In some embodiments, the heavy chain variable domain comprises SEQ ID NO: 35 and the light chain variable domain comprises SEQ ID NO: 36. In some embodiments, the heavy chain variable domain comprises SEQ ID NO: 1 and the light chain variable domain comprises SEQ ID NO: 2. In some embodiments, the heavy chain variable domain comprises SEQ ID NO: 23 and the light chain variable domain comprises SEQ ID NO: 24. In some embodiments, the heavy chain variable domain comprises SEQ ID NO: 13 and the light chain variable domain comprises SEQ ID NO: 14.

In some embodiments, the heavy chain variable domain comprises SEQ ID NO: 27 and the light chain variable domain comprises SEQ ID NO: 28. In some embodiments, the heavy chain variable domain comprises the CDR1, CDR2, and CDR3 regions of SEQ ID NO: 5. In some embodiments, the heavy chain variable domain comprises the CDR1, CDR2, and CDR3 regions of SEQ ID NO: 41. In some embodiments, the heavy chain variable domain comprises the CDR1, CDR2, and CDR3 regions of SEQ ID NO: 1. In some embodiments, the heavy chain variable domain comprises the CDR1, CDR2, and CDR3 regions of SEQ ID NO: 23. In some embodiments, the heavy chain variable domain comprises the CDR1, CDR2, and CDR3 regions of SEQ ID NO: 13. In some embodiments, the heavy chain variable domain comprises the CDR1, CDR2, and CDR3 regions of SEQ ID NO: 27. In some embodiments, the light chain variable domain comprises the CDR1, CDR2, and CDR3 regions of SEQ ID NO: 6. In some embodiments, the light chain variable domain comprises the CDR1, CDR2, and CDR3 regions of SEQ ID NO: 42. In some embodiments, the light chain variable domain comprises the CDR1, CDR2, and CDR3 regions of SEQ ID NO: 36. In some embodiments, the light chain variable domain comprises the CDR1, CDR2, and CDR3 regions of SEQ ID NO: 2. In some embodiments, the light chain variable domain comprises the CDR1, CDR2, and CDR3 regions of SEQ ID NO: 24.

In some embodiments, the light chain variable domain comprises the CDR1, CDR2, and CDR3 regions of SEQ ID NO: 14. In some embodiments, the light chain variable domain comprises the CDR1, CDR2, and CDR3 regions of SEQ ID NO: 28. In some embodiments, the heavy chain variable domain comprises the CDR1, CDR2, and CDR3 regions of SEQ ID NO: 5 and the light chain variable domain comprises the CDR1, CDR2, and CDR3 regions of SEQ ID NO: 6. In some embodiments, the heavy chain variable domain comprises the CDR1, CDR2, and CDR3 regions of SEQ ID NO: 41 and the light chain variable domain comprises the CDR1, CDR2, and CDR3 regions of SEQ ID NO: 42. In some embodiments, the heavy chain variable domain comprises the CDR1, CDR2, and CDR3 regions of SEQ ID NO: 35 and the light chain variable domain comprises the CDR1, CDR2, and CDR3 regions of SEQ ID NO: 36. In some embodiments, the heavy chain variable domain comprises the CDR1, CDR2, and CDR3 regions of SEQ ID NO: 1 and the light chain variable domain comprises the CDR1, CDR2, and CDR3 regions of SEQ ID NO: 2. In some embodiments, the heavy chain variable domain comprises the CDR1, CDR2, and CDR3 regions of SEQ ID NO: 23 and the light chain variable domain comprises the CDR1, CDR2, and CDR3 regions of SEQ ID NO: 24. In some embodiments, the heavy chain variable domain comprises the CDR1, CDR2, and CDR3 regions of SEQ ID NO: 13 and the light chain variable domain comprises the CDR1, CDR2, and CDR3 regions of SEQ ID NO: 14. In some embodiments, the heavy chain variable domain comprises the CDR1, CDR2, and CDR3 regions of SEQ ID NO: 27 and the light chain variable domain comprises the CDR1, CDR2, and CDR3 regions of SEQ ID NO: 28.

In some embodiments, the antibody neutralizes a coronavirus. In some embodiments, the coronavirus is a SARS-CoV-2 virus. In some embodiments, the coronavirus is a SARS-CoV-2 variant strain. In some embodiments, the variant strain is selected from the group consisting of WA1, B.1.1.7, B1.526, B1.351, P1, B1.352, and B.1.427. In some embodiments, the antibody specifically binds an epitope on the surface of the coronavirus. In some embodiments, the epitope comprises at least a portion of a coronavirus spike protein. In some embodiments, the at least a portion of a coronavirus spike protein comprises a receptor binding domain (RBD) of a spike protein. In some embodiments, the at least a portion of a coronavirus spike protein comprises a N-Terminal domain (NTD) of a spike protein. In some embodiments, the coronavirus is a SARS-CoV-2 coronavirus. In some embodiments, the antibody is administered in combination with a second pharmaceutical agent.

In certain aspects, the invention provides a bispecific molecule comprising a first polypeptide chain and a second polypeptide chain, wherein: the first polypeptide chain comprises: a first light chain comprising a variable domain and a constant domain; a first heavy chain comprising a variable domain and a constant domain; the second polypeptide chain comprises: a second light chain comprising a variable domain and a constant domain; and a second heavy chain comprising a variable domain and a constant domain. In some embodiments, the first polypeptide chain and the second polypeptide chain are covalently bonded to one another.

In some embodiments, the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 47, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 111, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 135, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 187, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 211. In some embodiments, the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 48, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 112, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 136, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 188, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 212.

In some embodiments, the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 49, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 113, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 137, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 189, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 213. In some embodiments, the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 50, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 114, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 138, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 190, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 214. In some embodiments, the variable domain of the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13.

In some embodiments, the variable domain of the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14. In some embodiments, the variable domain of the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 45, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43. In some embodiments, the variable domain of the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 46, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44.

In some embodiments, the variable domain of the first heavy chain comprises CDR1, CDR2, and CDR3 of SEQ ID NO: 47; CDR1, CDR2, and CDR3 of SEQ ID NO: 111; CDR1, CDR2, and CDR3 of SEQ ID NO: 135; CDR1, CDR2, and CDR3 of SEQ ID NO: 187; or CDR1, CDR2, and CDR3 of SEQ ID NO: 211. In some embodiments, the variable domain of the first light chain comprises CDR1, CDR2, and CDR3 of SEQ ID NO: 48, CDR1, CDR2, and CDR3 of SEQ ID NO: 112; CDR1, CDR2, and CDR3 of SEQ ID NO: 136; CDR1, CDR2, and CDR3 of SEQ ID NO: 188; or CDR1, CDR2, and CDR3 of SEQ ID NO: 212. In some embodiments, the variable domain of the second heavy chain comprises CDR1, CDR2, and CDR3 of SEQ ID NO: 49, CDR1, CDR2, and CDR3 of SEQ ID NO: 113; CDR1, CDR2, and CDR3 of SEQ ID NO: 137; CDR1, CDR2, and CDR3 of SEQ ID NO: 189; or CDR1, CDR2, and CDR3 of SEQ ID NO: 213. In some embodiments, the variable domain of the second light chain comprises CDR1, CDR2, and CDR3 SEQ ID NO: 50, CDR1, CDR2, and CDR3 of SEQ ID NO: 114; CDR1, CDR2, and CDR3 of SEQ ID NO: 138; CDR1, CDR2, and CDR3 of SEQ ID NO: 190; or CDR1, CDR2, and CDR3 of SEQ ID NO: 214.

In some embodiments, the molecule comprises SEQ ID NO: 47, SEQ ID NO: 48, SEQ ID NO: 49, and SEQ ID NO: 50. In some embodiments, the molecule comprises SEQ ID NO:111, SEQ ID NO: 112, SEQ ID NO: 113, and SEQ ID NO: 114. In some embodiments, the molecule comprises SEQ ID NO: 135, SEQ ID NO: 135, SEQ ID NO: 137, and SEQ ID NO: 138. In some embodiments, the molecule comprises SEQ ID NO: 187, SEQ ID NO: 188, SEQ ID NO: 189, and SEQ ID NO: 190. In some embodiments, the molecule comprises SEQ ID NO: 211, SEQ ID NO: 212, SEQ ID NO: 213, and SEQ ID NO: 214.

In some embodiments, the molecule further comprises one or more disulfide bonds. In some embodiments, the molecule further comprises one or more linker sequences. In some embodiments, the molecule is capable of neutralizing a coronavirus. In some embodiments, the coronavirus is a SARS-CoV-2 virus. In some embodiments, the SARS-CoV-2 virus strain is selected from the group consisting of WA1, B.1.1.7, B1.526, B1.351, P1, B1.352, and B.1.427. In some embodiments, the molecule specifically recognizes a first epitope and a second epitope on a SARS-CoV-2 particle. In some embodiments, the first epitope comprises at least a portion of a receptor binding domain (RBD) on a spike protein of a SARS-CoV-2 particle. In some embodiments, the second epitope comprises at least a portion of a N-terminal domain (NTD) on a spike protein of a SARS-CoV-2 particle.

In certain aspects, the invention provides a method of treating or preventing a COVID-19 infection in a subject in need thereof, the method comprising administering to the subject a composition comprising a bispecific molecule comprising a first polypeptide chain and a second polypeptide chain, wherein: the first polypeptide chain comprises: a first light chain comprising a variable domain and a constant domain; a first heavy chain comprising a variable domain and a constant domain; the second polypeptide chain comprises: a second light chain comprising a variable domain and a constant domain; and a second heavy chain comprising a variable domain and a constant domain. In some embodiments, the first polypeptide chain and the second polypeptide chain are covalently bonded to one another.

In some embodiments, the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 47, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 111, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 135, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 187, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 211.

In some embodiments, the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 48, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 112, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 136, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 188, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 212.

In some embodiments, the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 49, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 113, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 137, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 189, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 213.

In some embodiments, the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 50, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 114, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 138, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 190, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 214.

In some embodiments, the variable domain of the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13. In some embodiments, the variable domain of the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14. In some embodiments, the variable domain of the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 45, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43.

In some embodiments, the variable domain of the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 46, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44.

In some embodiments, the variable domain of the first heavy chain comprises CDR1, CDR2, and CDR3 of SEQ ID NO: 47; CDR1, CDR2, and CDR3 of SEQ ID NO: 111; CDR1, CDR2, and CDR3 of SEQ ID NO: 135; CDR1, CDR2, and CDR3 of SEQ ID NO: 187; or CDR1, CDR2, and CDR3 of SEQ ID NO: 211. In some embodiments, the variable domain of the first light chain comprises CDR1, CDR2, and CDR3 of SEQ ID NO: 48, CDR1, CDR2, and CDR3 of SEQ ID NO: 112; CDR1, CDR2, and CDR3 of SEQ ID NO: 136; CDR1, CDR2, and CDR3 of SEQ ID NO: 188; or CDR1, CDR2, and CDR3 of SEQ ID NO: 212. In some embodiments, the variable domain of the second heavy chain comprises CDR1, CDR2, and CDR3 of SEQ ID NO: 49, CDR1, CDR2, and CDR3 of SEQ ID NO: 113; CDR1, CDR2, and CDR3 of SEQ ID NO: 137; CDR1, CDR2, and CDR3 of SEQ ID NO: 189; or CDR1, CDR2, and CDR3 of SEQ ID NO: 213. In some embodiments, the variable domain of the second light chain comprises CDR1, CDR2, and CDR3 SEQ ID NO: 50, CDR1, CDR2, and CDR3 of SEQ ID NO: 114; CDR1, CDR2, and CDR3 of SEQ ID NO: 138; CDR1, CDR2, and CDR3 of SEQ ID NO: 190; or CDR1, CDR2, and CDR3 of SEQ ID NO: 214. In some embodiments, the molecule comprises SEQ ID NO: 47, SEQ ID NO: 48, SEQ ID NO: 49, and SEQ ID NO: 50. In some embodiments, the molecule comprises SEQ ID NO:111, SEQ ID NO: 112, SEQ ID NO: 113, and SEQ ID NO: 114. In some embodiments, the molecule comprises SEQ ID NO: 135, SEQ ID NO: 135, SEQ ID NO: 137, and SEQ ID NO: 138. In some embodiments, the molecule comprises SEQ ID NO: 187, SEQ ID NO: 188, SEQ ID NO: 189, and SEQ ID NO: 190. In some embodiments, the molecule comprises SEQ ID NO: 211, SEQ ID NO: 212, SEQ ID NO: 213, and SEQ ID NO: 214.

In some embodiments, the molecule further comprises one or more disulfide bonds. In some embodiments, the molecule further comprises one or more linker sequences. In some embodiments, the molecule is capable of neutralizing a coronavirus. In some embodiments, the coronavirus is a SARS-CoV-2 virus. In some embodiments, the SARS-CoV-2 virus strain is selected from the group consisting of WA1, B.1.1.7, B1.526, B1.351, P1, B1.352, and B.1.427.

In some embodiments, the molecule specifically recognizes a first epitope and a second epitope on a SARS-CoV-2 particle. In some embodiments, the first epitope comprises at least a portion of a receptor binding domain (RBD) on a spike protein of a SARS-CoV-2 particle. In some embodiments, the second epitope comprises at least a portion of a N-terminal domain (NTD) on a spike protein of a SARS-CoV-2 particle. In some embodiments, the composition is administered in combination with a second therapeutic agent.

In certain aspects, the invention provides a bispecific molecule comprising a first polypeptide chain and a second polypeptide chain, wherein: the first polypeptide chain comprises: a first light chain comprising a variable domain and a constant domain; a first heavy chain comprising a variable domain and a constant domain; the second polypeptide chain comprises: a second light chain comprising a variable domain and a constant domain; and a second heavy chain comprising a variable domain and a constant domain.

In some embodiments, the first polypeptide chain and the second polypeptide chain are covalently bonded to one another. In some embodiments, the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 59 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 63. In some embodiments, the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 60 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 64. In some embodiments, the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 61 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 65. In some embodiments, the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 62 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 66.

In some embodiments, the first heavy chain comprises SEQ ID NO: 59 or SEQ ID NO: 63. In some embodiments, the first light chain comprises SEQ ID NO: 60 or SEQ ID NO: 64. In some embodiments, the second heavy chain comprises SEQ ID NO: 61 or SEQ ID NO: 65. In some embodiments, the second light chain comprises SEQ ID NO: 62 or SEQ ID NO: 66.

In some embodiments, the variable domain of the first heavy chain comprises CDR1, CDR2, and CDR3 of SEQ ID NO: 59 or CDR1, CDR2, and CDR3 of SEQ ID NO: 63. In some embodiments, the variable domain of the first light chain comprises CDR1, CDR2, and CDR3 of SEQ ID NO: 60 or CDR1, CDR2, and CDR3 of SEQ ID NO: 64. In some embodiments, the variable domain of the second heavy chain comprises CDR1, CDR2, and CDR3 of SEQ ID NO: 61 or CDR1, CDR2, and CDR3 of SEQ ID NO: 65. In some embodiments, the variable domain of the second light chain comprises CDR1, CDR2, and CDR3 SEQ ID NO: 62 or CDR1, CDR2, and CDR3 of SEQ ID NO: 66.

In some embodiments, the variable domain of the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27. In some embodiments, the variable domain of the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28. In some embodiments, the variable domain of the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13. In some embodiments, the variable domain of the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14.

In some embodiments, the variable domain of the first heavy chain comprises SEQ ID NO: 13 or SEQ ID NO: 27. In some embodiments, the variable domain of the first light chain comprises SEQ ID NO: 14 or SEQ ID NO: 28. In some embodiments, the variable domain of the second heavy chain comprises SEQ ID NO: 27 or SEQ ID NO: 13. In some embodiments, the variable domain of the second light chain comprises SEQ ID NO: 28 or SEQ ID NO: 14.

In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 59, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 60, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 61, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 62. In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 63, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 64, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 65, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 66. In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 13, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28.

In some embodiments, the molecule comprises SEQ ID NOs: 59, 60, 61, and 62. In some embodiments, the molecule comprises SEQ ID NOs: 63, 64, 65, and 66. In some embodiments, the molecule comprises SEQ ID NOs: 13, 14, 27, and 28.

In some embodiments, the molecule further comprises one or more disulfide bonds. In some embodiments, the molecule further comprises one or more linker sequences. In some embodiments, the molecule is capable of neutralizing a coronavirus. In some embodiments, the coronavirus is a SARS-CoV-2 virus strain. In some embodiments, the molecule specifically recognizes a first epitope and a second epitope on a SARS-CoV-2 particle. In some embodiments, the first epitope comprises at least a portion of a receptor binding domain (RBD) on an S protein of a SARS-CoV-2 particle. In some embodiments, the second epitope comprises at least a portion of a N-terminal domain (NTD) on an S protein of a SARS-CoV-2 particle.

In certain aspects, the invention provides a method of treating a COVID-19 infection in a subject in need thereof, the method comprising administering to the subject a composition comprising a bispecific molecule, wherein the bispecific molecule comprises a first polypeptide chain and a second polypeptide chain, wherein: the first polypeptide chain comprises: a first light chain comprising a variable domain and a constant domain; a first heavy chain comprising a variable domain and a constant domain; the second polypeptide chain comprises: a second light chain comprising a variable domain and a constant domain; and a second heavy chain comprising a variable domain and a constant domain.

In some embodiments, the first polypeptide chain and the second polypeptide chain are covalently bonded to one another. In some embodiments, the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 59 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 63. In some embodiments, the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 60 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 64. In some embodiments, the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 61 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 65. In some embodiments, the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 62 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 66.

In some embodiments, the first heavy chain comprises SEQ ID NO: 59 or SEQ ID NO: 63. In some embodiments, the first light chain comprises SEQ ID NO: 60 or SEQ ID NO: 64. In some embodiments, the second heavy chain comprises SEQ ID NO: 61 or SEQ ID NO: 65. In some embodiments, the second light chain comprises SEQ ID NO: 62 or SEQ ID NO: 66. In some embodiments, the variable domain of the first heavy chain comprises CDR1, CDR2, and CDR3 of SEQ ID NO: 59 or CDR1, CDR2, and CDR3 of SEQ ID NO: 63. In some embodiments, the variable domain of the first light chain comprises CDR1, CDR2, and CDR3 of SEQ ID NO: 60 or CDR1, CDR2, and CDR3 of SEQ ID NO: 64. In some embodiments, the variable domain of the second heavy chain comprises CDR1, CDR2, and CDR3 of SEQ ID NO: 61 or CDR1, CDR2, and CDR3 of SEQ ID NO: 65. In some embodiments, the variable domain of the second light chain comprises CDR1, CDR2, and CDR3 SEQ ID NO: 62 or CDR1, CDR2, and CDR3 of SEQ ID NO: 66.

In some embodiments, the variable domain of the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27. In some embodiments, the variable domain of the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28. In some embodiments, the variable domain of the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13. In some embodiments, the variable domain of the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14.

In some embodiments, the variable domain of the first heavy chain comprises SEQ ID NO: 13 or SEQ ID NO: 27. In some embodiments, the variable domain of the first light chain comprises SEQ ID NO: 14 or SEQ ID NO: 28. In some embodiments, the variable domain of the second heavy chain comprises SEQ ID NO: 27 or SEQ ID NO: 13. In some embodiments, the variable domain of the second light chain comprises SEQ ID NO: 28 or SEQ ID NO: 14.

In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 59, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 60, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 61, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 62. In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 63, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 64, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 65, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 66. In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 13, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28.

In some embodiments, the molecule comprises SEQ ID NOs: 59, 60, 61, and 62. In some embodiments, the molecule comprises SEQ ID NOs: 63, 64, 65, and 66. In some embodiments, the molecule comprises SEQ ID NOs: 13, 14, 27, and 28.

In some embodiments, the molecule further comprises one or more disulfide bonds. In some embodiments, the molecule further comprises one or more linker sequences. In some embodiments, the molecule is capable of neutralizing a coronavirus. In some embodiments, the coronavirus is a SARS-CoV-2 virus strain. In some embodiments, the molecule specifically recognizes a first epitope and a second epitope on a SARS-CoV-2 particle. In some embodiments, the first epitope comprises at least a portion of a receptor binding domain (RBD) on a spike protein of a SARS-CoV-2 particle. In some embodiments, the second epitope comprises at least a portion of a N-terminal domain (NTD) on a spike protein of a SARS-CoV-2 particle. In some embodiments, the composition is administered in combination with a second therapeutic agent.

In certain aspects, the invention provides a method of preventing a COVID-19 infection in a subject in need thereof, the method comprising administering to the subject a composition comprising a bispecific molecule, wherein the bispecific molecule comprises a first polypeptide chain and a second polypeptide chain, wherein: the first polypeptide chain comprises: a first light chain comprising a variable domain and a constant domain; a first heavy chain comprising a variable domain and a constant domain; the second polypeptide chain comprises: a second light chain comprising a variable domain and a constant domain; and a second heavy chain comprising a variable domain and a constant domain.

In some embodiments, the first polypeptide chain and the second polypeptide chain are covalently bonded to one another. In some embodiments, the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 59 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 63. In some embodiments, the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 60 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 64. In some embodiments, the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 61 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 65. In some embodiments, the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 62 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 66.

In some embodiments, the first heavy chain comprises SEQ ID NO: 59 or SEQ ID NO: 63. In some embodiments, the first light chain comprises SEQ ID NO: 60 or SEQ ID NO: 64. In some embodiments, the second heavy chain comprises SEQ ID NO: 61 or SEQ ID NO: 65. In some embodiments, the second light chain comprises SEQ ID NO: 62 or SEQ ID NO: 66.

In some embodiments, the variable domain of the first heavy chain comprises CDR1, CDR2, and CDR3 of SEQ ID NO: 59 or CDR1, CDR2, and CDR3 of SEQ ID NO: 63. In some embodiments, the variable domain of the first light chain comprises CDR1, CDR2, and CDR3 of SEQ ID NO: 60 or CDR1, CDR2, and CDR3 of SEQ ID NO: 64. In some embodiments, the variable domain of the second heavy chain comprises CDR1, CDR2, and CDR3 of SEQ ID NO: 61 or CDR1, CDR2, and CDR3 of SEQ ID NO: 65. In some embodiments, the variable domain of the second light chain comprises CDR1, CDR2, and CDR3 SEQ ID NO: 62 or CDR1, CDR2, and CDR3 of SEQ ID NO: 66.

In some embodiments, the variable domain of the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27. In some embodiments, the variable domain of the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28. In some embodiments, the variable domain of the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13. In some embodiments, the variable domain of the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28 or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14.

In some embodiments, the variable domain of the first heavy chain comprises SEQ ID NO: 13 or SEQ ID NO: 27. In some embodiments, the variable domain of the first light chain comprises SEQ ID NO: 14 or SEQ ID NO: 28. In some embodiments, the variable domain of the second heavy chain comprises SEQ ID NO: 27 or SEQ ID NO: 13. In some embodiments, the variable domain of the second light chain comprises SEQ ID NO: 28 or SEQ ID NO: 14.

In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 59, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 60, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 61, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 62. In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 63, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 64, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 65, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 66. In some embodiments, the molecule comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NOs: 13, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27, and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28.

In some embodiments, the molecule comprises SEQ ID NOs: 59, 60, 61, and 62. In some embodiments, the molecule comprises SEQ ID NOs: 63, 64, 65, and 66. In some embodiments, the molecule comprises SEQ ID NOs: 13, 14, 27, and 28.

In some embodiments, the molecule further comprises one or more disulfide bonds. In some embodiments, the molecule further comprises one or more linker sequences. In some embodiments, the molecule is capable of neutralizing a coronavirus. In some embodiments, the coronavirus is a SARS-CoV-2 virus strain. In some embodiments, the molecule specifically recognizes a first epitope and a second epitope on a SARS-CoV-2 particle. In some embodiments, the first epitope comprises at least a portion of a receptor binding domain (RBD) on an S protein of a SARS-CoV-2 particle. In some embodiments, the second epitope comprises at least a portion of a N-terminal domain (NTD) on an S protein of a SARS-CoV-2 particle. In some embodiments, the composition is administered in combination with a second therapeutic agent.

In certain aspects, the invention provides a synthesized monoclonal antibody, or a functional fragment thereof, comprising a heavy chain having a variable domain and a constant domain and a light chain having a variable domain and a constant domain, wherein the antibody binds an antigen on a coronavirus particle.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the heavy chain variable domain comprises SEQ ID NO: 15. In some embodiments, the light chain variable domain comprises SEQ ID NO: 16.

In some embodiments, the antibody comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15 and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the antibody comprises SEQ ID NO: 15 and SEQ ID NO: 16.

In some embodiments, the heavy chain variable domain comprises the CDR1, CDR2, and CDR3 regions of SEQ ID NO: 15. In some embodiments, the light chain variable domain comprises the CDR1, CDR2, and CDR3 regions of SEQ ID NO: 16.

In some embodiments, the antibody neutralizes a coronavirus. In some embodiments, the coronavirus is SARS-CoV-2. In some embodiments, the antibody specifically binds an epitope on the surface of the coronavirus. In some embodiments, the epitope comprises at least a portion of a coronavirus spike protein. In some embodiments, the at least a portion of a coronavirus spike protein comprises a receptor binding domain (RBD) of a spike protein. In some embodiments, the coronavirus is a SARS-CoV-2 coronavirus.

In certain aspects, the invention provides a method of treating a COVID-19 infection in a subject in need thereof, the method comprising administering to the subject a therapeutically effective amount of a synthesized monoclonal antibody, or a functional fragment thereof, comprising a heavy chain having a variable domain and a constant domain and a light chain having a variable domain and a constant domain, wherein the antibody binds an antigen on a coronavirus particle.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the heavy chain variable domain comprises SEQ ID NO: 15. In some embodiments, the light chain variable domain comprises SEQ ID NO: 16.

In some embodiments, the antibody comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15 and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the antibody comprises SEQ ID NO: 15 and SEQ ID NO: 16.

In some embodiments, the heavy chain variable domain comprises the CDR1, CDR2, and CDR3 regions of SEQ ID NO: 15. In some embodiments, the light chain variable domain comprises the CDR1, CDR2, and CDR3 regions of SEQ ID NO: 16.

In some embodiments, the antibody neutralizes a coronavirus. In some embodiments, the coronavirus is SARS-CoV-2. In some embodiments, the antibody neutralizes a coronavirus. In some embodiments, the coronavirus is SARS-CoV-2. In some embodiments, the antibody specifically binds an epitope on the surface of the coronavirus. In some embodiments, the epitope comprises at least a portion of a coronavirus spike protein. In some embodiments, the at least a portion of a coronavirus spike protein comprises a receptor binding domain (RBD) of a spike protein. In some embodiments, the coronavirus is a SARS-CoV-2 coronavirus. In some embodiments, the antibody is administered in combination with a second pharmaceutical agent.

In certain aspects, the invention provides a method of preventing a COVID-19 infection in a subject in need thereof, the method comprising administering to the subject a therapeutically effective amount of a synthesized monoclonal antibody, or a functional fragment thereof, comprising a heavy chain having a variable domain and a constant domain and a light chain having a variable and a constant domain, wherein the antibody binds an antigen on a coronavirus particle.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the heavy chain variable domain comprises SEQ ID NO: 15. In some embodiments, the light chain variable domain comprises SEQ ID NO: 16.

In some embodiments, the antibody comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15 and a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the antibody comprises SEQ ID NO: 15 and SEQ ID NO: 16.

In some embodiments, the heavy chain variable domain comprises the CDR1, CDR2, and CDR3 regions of SEQ ID NO: 15. In some embodiments, the light chain variable domain comprises the CDR1, CDR2, and CDR3 regions of SEQ ID NO: 16.

In some embodiments, the antibody neutralizes a coronavirus. In some embodiments, the coronavirus is SARS-CoV-2. In some embodiments, the antibody neutralizes a coronavirus. In some embodiments, the coronavirus is SARS-CoV-2. In some embodiments, the antibody specifically binds an epitope on the surface of the coronavirus. In some embodiments, the epitope comprises at least a portion of a coronavirus spike protein. In some embodiments, the at least a portion of a coronavirus spike protein comprises a receptor binding domain (RBD) of a spike protein. In some embodiments, the coronavirus is a SARS-CoV-2 coronavirus. In some embodiments, the antibody is administered in combination with a second pharmaceutical agent.

In certain aspects, the invention provides a synthesized monoclonal antibody, or a functional fragment thereof, comprising a heavy chain having a variable domain and a constant domain and a light chain having a variable domain and a constant domain, wherein the antibody binds an antigen on a coronavirus particle.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 29, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 31.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 33, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 39, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 45.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 30, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 32.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 34, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 40, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 46.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 29 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 30.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 31 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 32. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 33 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 34. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 39 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 40. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 45 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 46.

In some embodiments, the heavy chain variable domain comprises SEQ ID NO: 3 and the light chain variable domain comprises SEQ ID NO: 4. In some embodiments, the heavy chain variable domain comprises SEQ ID NO: 7 and the light chain variable domain comprises SEQ ID NO: 8. In some embodiments, the heavy chain variable domain comprises SEQ ID NO: 9 and the light chain variable domain comprises SEQ ID NO: 10. In some embodiments, the heavy chain variable domain comprises SEQ ID NO: 11 and the light chain variable domain comprises SEQ ID NO: 12. In some embodiments, the heavy chain variable domain comprises SEQ ID NO: 17 and the light chain variable domain comprises SEQ ID NO: 18. In some embodiments, the heavy chain variable domain comprises SEQ ID NO: 19 and the light chain variable domain comprises SEQ ID NO: 20. In some embodiments, the heavy chain variable domain comprises SEQ ID NO: 21 and the light chain variable domain comprises SEQ ID NO: 22. In some embodiments, the heavy chain variable domain comprises SEQ ID NO: 25 and the light chain variable domain comprises SEQ ID NO: 26. In some embodiments, the heavy chain variable domain comprises SEQ ID NO: 29 and the light chain variable domain comprises SEQ ID NO: 30. In some embodiments, the heavy chain variable domain comprises SEQ ID NO: 31 and the light chain variable domain comprises SEQ ID NO: 32. In some embodiments, the heavy chain variable domain comprises SEQ ID NO: 33 and the light chain variable domain comprises SEQ ID NO: 34. In some embodiments, the heavy chain variable domain comprises SEQ ID NO: 37 and the light chain variable domain comprises SEQ ID NO: 38. In some embodiments, the heavy chain variable domain comprises SEQ ID NO: 39 and the light chain variable domain comprises SEQ ID NO: 40. In some embodiments, the heavy chain variable domain comprises SEQ ID NO: 43 and the light chain variable domain comprises SEQ ID NO: 44. In some embodiments, the heavy chain variable domain comprises SEQ ID NO: 45 and the light chain variable domain comprises SEQ ID NO: 46.

In some embodiments, the heavy chain variable domain comprises the CDR1, CDR2, and CDR3 regions of SEQ ID NO: 3; the CDR1, CDR2, and CDR3 regions of SEQ ID NO: 7; the CDR1, CDR2, and CDR3 regions of SEQ ID NO: 9; the CDR1, CDR2, and CDR3 regions of SEQ ID NO: 11; or the CDR1, CDR2, and CDR3 regions of SEQ ID NO: 17. In some embodiments, the heavy chain variable domain comprises the CDR1, CDR2, and CDR3 regions of SEQ ID NO: 19; the CDR1, CDR2, and CDR3 regions of SEQ ID NO: 21; the CDR1, CDR2, and CDR3 regions of SEQ ID NO: 25; the CDR1, CDR2, and CDR3 regions of SEQ ID NO: 29; or the CDR1, CDR2, and CDR3 regions of SEQ ID NO: 31. In some embodiments, the heavy chain variable domain comprises the CDR1, CDR2, and CDR3 regions of SEQ ID NO: 33; the CDR1, CDR2, and CDR3 regions of SEQ ID NO: 37; the CDR1, CDR2, and CDR3 regions of SEQ ID NO: 39; the CDR1, CDR2, and CDR3 regions of SEQ ID NO: 43; or the CDR1, CDR2, and CDR3 regions of SEQ ID NO: 45. In some embodiments, the light chain variable domain comprises the CDR1, CDR2, and CDR3 regions of SEQ ID NO: 4; the CDR1, CDR2, and CDR3 regions of SEQ ID NO: 8; the CDR1, CDR2, and CDR3 regions of SEQ ID NO: 10; the CDR1, CDR2, and CDR3 regions of SEQ ID NO: 12; or the CDR1, CDR2, and CDR3 regions of SEQ ID NO: 18. In some embodiments, the light chain variable domain comprises the CDR1, CDR2, and CDR3 regions of SEQ ID NO: 20; the CDR1, CDR2, and CDR3 regions of SEQ ID NO: 22; the CDR1, CDR2, and CDR3 regions of SEQ ID NO: 26; the CDR1, CDR2, and CDR3 regions of SEQ ID NO: 30; or the CDR1, CDR2, and CDR3 regions of SEQ ID NO: 32. In some embodiments, the light chain variable domain comprises the CDR1, CDR2, and CDR3 regions of SEQ ID NO: 34; the CDR1, CDR2, and CDR3 regions of SEQ ID NO: 38; the CDR1, CDR2, and CDR3 regions of SEQ ID NO: 40; the CDR1, CDR2, and CDR3 regions of SEQ ID NO: 44; or the CDR1, CDR2, and CDR3 regions of SEQ ID NO: 46.

In some embodiments, the antibody neutralizes a coronavirus. In some embodiments, the coronavirus is a SARS-CoV-2 virus. In some embodiments, the coronavirus is a SARS-CoV-2 variant strain. In some embodiments, the variant strain is selected from the group consisting of WA1, B.1.1.7, B1.526, B1.351, P1, B1.352, and B.1.427.

In some embodiments, the antibody specifically binds an epitope on the surface of the coronavirus. In some embodiments, the epitope comprises at least a portion of a coronavirus spike protein. In some embodiments, the at least a portion of a coronavirus spike protein comprises a receptor binding domain (RBD) of a spike protein. In some embodiments, the at least a portion of a coronavirus spike protein comprises a N-Terminal domain (NTD) of a spike protein. In some embodiments, the coronavirus is a SARS-CoV-2 coronavirus.

In certain aspects, the invention provides a method of treating or preventing a COVID-19 infection in a subject in need thereof, the method comprising administering to the subject a therapeutically effective amount of a synthesized monoclonal antibody, or a functional fragment thereof, comprising a heavy chain having a variable domain and a constant domain and a light chain having a variable domain and a constant domain, wherein the antibody binds an antigen on a coronavirus particle.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 29, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 31.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 33, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 39, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 45.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 30, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 32.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 34, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 40, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 46.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 29 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 30. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 31 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 32. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 33 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 34. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 39 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 40. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 45 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 46.

In some embodiments, the heavy chain variable domain comprises SEQ ID NO: 3 and the light chain variable domain comprises SEQ ID NO: 4. In some embodiments, the heavy chain variable domain comprises SEQ ID NO: 7 and the light chain variable domain comprises SEQ ID NO: 8. In some embodiments, the heavy chain variable domain comprises SEQ ID NO: 9 and the light chain variable domain comprises SEQ ID NO: 10. In some embodiments, the heavy chain variable domain comprises SEQ ID NO: 11 and the light chain variable domain comprises SEQ ID NO: 12. In some embodiments, the heavy chain variable domain comprises SEQ ID NO: 17 and the light chain variable domain comprises SEQ ID NO: 18. In some embodiments, the heavy chain variable domain comprises SEQ ID NO: 19 and the light chain variable domain comprises SEQ ID NO: 20. In some embodiments, the heavy chain variable domain comprises SEQ ID NO: 21 and the light chain variable domain comprises SEQ ID NO: 22. In some embodiments, the heavy chain variable domain comprises SEQ ID NO: 25 and the light chain variable domain comprises SEQ ID NO: 26. In some embodiments, the heavy chain variable domain comprises SEQ ID NO: 29 and the light chain variable domain comprises SEQ ID NO: 30. In some embodiments, the heavy chain variable domain comprises SEQ ID NO: 31 and the light chain variable domain comprises SEQ ID NO: 32. In some embodiments, the heavy chain variable domain comprises SEQ ID NO: 33 and the light chain variable domain comprises SEQ ID NO: 34. In some embodiments, the heavy chain variable domain comprises SEQ ID NO: 37 and the light chain variable domain comprises SEQ ID NO: 38. In some embodiments, the heavy chain variable domain comprises SEQ ID NO: 39 and the light chain variable domain comprises SEQ ID NO: 40. In some embodiments, the heavy chain variable domain comprises SEQ ID NO: 43 and the light chain variable domain comprises SEQ ID NO: 44. In some embodiments, the heavy chain variable domain comprises SEQ ID NO: 45 and the light chain variable domain comprises SEQ ID NO: 46.

In some embodiments, the heavy chain variable domain comprises the CDR1, CDR2, and CDR3 regions of SEQ ID NO: 3; the CDR1, CDR2, and CDR3 regions of SEQ ID NO: 7; the CDR1, CDR2, and CDR3 regions of SEQ ID NO: 9; the CDR1, CDR2, and CDR3 regions of SEQ ID NO: 11; or the CDR1, CDR2, and CDR3 regions of SEQ ID NO: 17. In some embodiments, the heavy chain variable domain comprises the CDR1, CDR2, and CDR3 regions of SEQ ID NO: 19; the CDR1, CDR2, and CDR3 regions of SEQ ID NO: 21; the CDR1, CDR2, and CDR3 regions of SEQ ID NO: 25; the CDR1, CDR2, and CDR3 regions of SEQ ID NO: 29; or the CDR1, CDR2, and CDR3 regions of SEQ ID NO: 31. In some embodiments, the heavy chain variable domain comprises the CDR1, CDR2, and CDR3 regions of SEQ ID NO: 33; the CDR1, CDR2, and CDR3 regions of SEQ ID NO: 37; the CDR1, CDR2, and CDR3 regions of SEQ ID NO: 39; the CDR1, CDR2, and CDR3 regions of SEQ ID NO: 43; or the CDR1, CDR2, and CDR3 regions of SEQ ID NO: 45. In some embodiments, the light chain variable domain comprises the CDR1, CDR2, and CDR3 regions of SEQ ID NO: 4; the CDR1, CDR2, and CDR3 regions of SEQ ID NO: 8; the CDR1, CDR2, and CDR3 regions of SEQ ID NO: 10; the CDR1, CDR2, and CDR3 regions of SEQ ID NO: 12; or the CDR1, CDR2, and CDR3 regions of SEQ ID NO: 18. In some embodiments, the light chain variable domain comprises the CDR1, CDR2, and CDR3 regions of SEQ ID NO: 20; the CDR1, CDR2, and CDR3 regions of SEQ ID NO: 22; the CDR1, CDR2, and CDR3 regions of SEQ ID NO: 26; the CDR1, CDR2, and CDR3 regions of SEQ ID NO: 30; or the CDR1, CDR2, and CDR3 regions of SEQ ID NO: 32. In some embodiments, the light chain variable domain comprises the CDR1, CDR2, and CDR3 regions of SEQ ID NO: 34; the CDR1, CDR2, and CDR3 regions of SEQ ID NO: 38; the CDR1, CDR2, and CDR3 regions of SEQ ID NO: 40; the CDR1, CDR2, and CDR3 regions of SEQ ID NO: 44; or the CDR1, CDR2, and CDR3 regions of SEQ ID NO: 46.

In some embodiments, the antibody neutralizes a coronavirus. In some embodiments, the coronavirus is a SARS-CoV-2 virus. In some embodiments, the coronavirus is a SARS-CoV-2 variant strain. In some embodiments, the variant strain is selected from the group consisting of WA1, B.1.1.7, B1.526, B1.351, P1, B1.352, and B.1.427.

In some embodiments, the antibody specifically binds an epitope on the surface of the coronavirus. In some embodiments, the epitope comprises at least a portion of a coronavirus spike protein. In some embodiments, the at least a portion of a coronavirus spike protein comprises a receptor binding domain (RBD) of a spike protein. In some embodiments, the at least a portion of a coronavirus spike protein comprises a N-Terminal domain (NTD) of a spike protein. In some embodiments, the coronavirus is a SARS-CoV-2 coronavirus. In some embodiments, the composition is administered in combination with a second therapeutic agent.

In certain aspects, the invention provides a bispecific molecule comprising a first polypeptide chain and a second polypeptide chain, wherein: the first polypeptide chain comprises: a first light chain comprising a variable domain and a constant domain; a first heavy chain comprising a variable domain and a constant domain; the second polypeptide chain comprises: a second light chain comprising a variable domain and a constant domain; and a second heavy chain comprising a variable domain and a constant domain.

In some embodiments, the first polypeptide chain and the second polypeptide chain are covalently bonded to one another. In some embodiments, the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 51, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 55, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 67, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 71, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 75, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 79, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 83, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 87, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 91, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 95, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 99, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 103, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 107, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 115, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 119, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 123, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 127, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 131, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 139, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 143, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 147, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 151, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 155, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 159, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 163, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 167, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 171, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 175, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 179, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO:183.

In some embodiments, the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 52, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 56, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 68; a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 72, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 76, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 80, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 84, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 88, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 92, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 96, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 100, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 104, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 108, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 116, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 120, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 124, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 128, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 132, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 140, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 144, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 148, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 152, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 156, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 160, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 164, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 168, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 172, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 176, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 180, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO:184.

In some embodiments, the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 53, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 57, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 69, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 73, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 77, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 81, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 85, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 89, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 93, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 97, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 101, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 105, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 109, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 117, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 121, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 125, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 129, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 133, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 141, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 145, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 149, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 153, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 157, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 161, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 165, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 169, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 173, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 177, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 181, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 185.

In some embodiments, the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 54, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 58, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 70, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 74, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 78, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 82, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 86, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 90, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 94, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 98, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 102, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 106, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 110, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 118, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 122, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 126, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 130, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 134, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 142, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 146, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 150, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 154, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 158, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 162, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 166, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 170, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 174, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 178, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 182, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 186.

In some embodiments, variable domain of the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 41.

In some embodiments, variable domain of the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 42.

In some embodiments, variable domain of the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 29, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 23, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 39, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9.

In some embodiments, variable domain of the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 30, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 24, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 40, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10.

In some embodiments, the variable domain of the first heavy chain comprises the CDR1, CDR2, and CDR3 of SEQ ID NO: 51; the CDR1, CDR2, and CDR3 of SEQ ID NO: 55; the CDR1, CDR2, and CDR3 of SEQ ID NO: 67; the CDR1, CDR2, and CDR3 of SEQ ID NO: 71; the CDR1, CDR2, and CDR3 of SEQ ID NO: 75; the CDR1, CDR2, and CDR3 of SEQ ID NO: 79; the CDR1, CDR2, and CDR3 of SEQ ID NO: 83; the CDR1, CDR2, and CDR3 of SEQ ID NO: 87; the CDR1, CDR2, and CDR3 of SEQ ID NO: 91; the CDR1, CDR2, and CDR3 of SEQ ID NO: 95; the CDR1, CDR2, and CDR3 of SEQ ID NO: 99; the CDR1, CDR2, and CDR3 of SEQ ID NO: 103; the CDR1, CDR2, and CDR3 of SEQ ID NO: 107; the CDR1, CDR2, and CDR3 of SEQ ID NO: 115; the CDR1, CDR2, and CDR3 of SEQ ID NO: 119, the CDR1, CDR2, and CDR3 of SEQ ID NO: 123, the CDR1, CDR2, and CDR3 of SEQ ID NO: 127; the CDR1, CDR2, and CDR3 of SEQ ID NO: 131; the CDR1, CDR2, and CDR3 of SEQ ID NO: 139; the CDR1, CDR2, and CDR3 of SEQ ID NO: 143; the CDR1, CDR2, and CDR3 of SEQ ID NO: 147; the CDR1, CDR2, and CDR3 of SEQ ID NO: 151; the CDR1, CDR2, and CDR3 of SEQ ID NO: 155; the CDR1, CDR2, and CDR3 of SEQ ID NO: 159; the CDR1, CDR2, and CDR3 of SEQ ID NO: 163; the CDR1, CDR2, and CDR3 of SEQ ID NO: 167, the CDR1, CDR2, and CDR3 of SEQ ID NO: 171, the CDR1, CDR2, and CDR3 of SEQ ID NO: 175, the CDR1, CDR2, and CDR3 of SEQ ID NO: 179, or the CDR1, CDR2, and CDR3 of SEQ ID NO: 183.

In some embodiments, the variable domain of the first light chain comprises the CDR1, CDR2, and CDR3 of SEQ ID NO: 52; the CDR1, CDR2, and CDR3 of SEQ ID NO: 56; the CDR1, CDR2, and CDR3 of SEQ ID NO: 68; the CDR1, CDR2, and CDR3 of SEQ ID NO: 72; the CDR1, CDR2, and CDR3 of SEQ ID NO: 76; the CDR1, CDR2, and CDR3 of SEQ ID NO: 80; the CDR1, CDR2, and CDR3 of SEQ ID NO: 84; the CDR1, CDR2, and CDR3 of SEQ ID NO: 88; the CDR1, CDR2, and CDR3 of SEQ ID NO: 92; the CDR1, CDR2, and CDR3 of SEQ ID NO: 96; the CDR1, CDR2, and CDR3 of SEQ ID NO: 100; the CDR1, CDR2, and CDR3 of SEQ ID NO: 104; the CDR1, CDR2, and CDR3 of SEQ ID NO: 108; the CDR1, CDR2, and CDR3 of SEQ ID NO: 116; the CDR1, CDR2, and CDR3 of SEQ ID NO: 120; the CDR1, CDR2, and CDR3 of SEQ ID NO: 124; the CDR1, CDR2, and CDR3 of SEQ ID NO: 128; the CDR1, CDR2, and CDR3 of SEQ ID NO: 132; the CDR1, CDR2, and CDR3 of SEQ ID NO: 140; the CDR1, CDR2, and CDR3 of SEQ ID NO: 144; the CDR1, CDR2, and CDR3 of SEQ ID NO: 148; the CDR1, CDR2, and CDR3 of SEQ ID NO: 152; the CDR1, CDR2, and CDR3 of SEQ ID NO: 156; the CDR1, CDR2, and CDR3 of SEQ ID NO: 160; the CDR1, CDR2, and CDR3 of SEQ ID NO: 164; the CDR1, CDR2, and CDR3 of SEQ ID NO: 168, the CDR1, CDR2, and CDR3 of SEQ ID NO: 172, the CDR1, CDR2, and CDR3 of SEQ ID NO: 176, the CDR1, CDR2, and CDR3 of SEQ ID NO: 180, or the CDR1, CDR2, and CDR3 of SEQ ID NO: 184.

In some embodiments, the variable domain of the second heavy chain comprises the CDR1, CDR2, and CDR3 of SEQ ID NO: 53; the CDR1, CDR2, and CDR3 of SEQ ID NO: 57; the CDR1, CDR2, and CDR3 of SEQ ID NO: 69; the CDR1, CDR2, and CDR3 of SEQ ID NO: 73; the CDR1, CDR2, and CDR3 of SEQ ID NO: 77; the CDR1, CDR2, and CDR3 of SEQ ID NO: 81; the CDR1, CDR2, and CDR3 of SEQ ID NO: 85; the CDR1, CDR2, and CDR3 of SEQ ID NO: 89; the CDR1, CDR2, and CDR3 of SEQ ID NO: 93; the CDR1, CDR2, and CDR3 of SEQ ID NO: 97; the CDR1, CDR2, and CDR3 of SEQ ID NO: 101; the CDR1, CDR2, and CDR3 of SEQ ID NO: 105; the CDR1, CDR2, and CDR3 of SEQ ID NO: 109; the CDR1, CDR2, and CDR3 of SEQ ID NO: 117; the CDR1, CDR2, and CDR3 of SEQ ID NO: 121; the CDR1, CDR2, and CDR3 of SEQ ID NO: 125; the CDR1, CDR2, and CDR3 of SEQ ID NO: 129; the CDR1, CDR2, and CDR3 of SEQ ID NO: 133; the CDR1, CDR2, and CDR3 of SEQ ID NO: 141; the CDR1, CDR2, and CDR3 of SEQ ID NO: 145; the CDR1, CDR2, and CDR3 of SEQ ID NO: 149; the CDR1, CDR2, and CDR3 of SEQ ID NO: 153; the CDR1, CDR2, and CDR3 of SEQ ID NO: 157; the CDR1, CDR2, and CDR3 of SEQ ID NO: 161; the CDR1, CDR2, and CDR3 of SEQ ID NO: 165; the CDR1, CDR2, and CDR3 of SEQ ID NO: 169, the CDR1, CDR2, and CDR3 of SEQ ID NO: 173, the CDR1, CDR2, and CDR3 of SEQ ID NO: 177, the CDR1, CDR2, and CDR3 of SEQ ID NO: 181, or the CDR1, CDR2, and CDR3 of SEQ ID NO: 185.

In some embodiments, the variable domain of the second light chain comprises the CDR1, CDR2, and CDR3 of SEQ ID NO: 54; the CDR1, CDR2, and CDR3 of SEQ ID NO: 58; the CDR1, CDR2, and CDR3 of SEQ ID NO: 70; the CDR1, CDR2, and CDR3 of SEQ ID NO: 74; the CDR1, CDR2, and CDR3 of SEQ ID NO: 78; the CDR1, CDR2, and CDR3 of SEQ ID NO: 82; the CDR1, CDR2, and CDR3 of SEQ ID NO: 86; the CDR1, CDR2, and CDR3 of SEQ ID NO: 90; the CDR1, CDR2, and CDR3 of SEQ ID NO: 94; the CDR1, CDR2, and CDR3 of SEQ ID NO: 98; the CDR1, CDR2, and CDR3 of SEQ ID NO: 102; the CDR1, CDR2, and CDR3 of SEQ ID NO: 106; the CDR1, CDR2, and CDR3 of SEQ ID NO: 110; the CDR1, CDR2, and CDR3 of SEQ ID NO: 118; the CDR1, CDR2, and CDR3 of SEQ ID NO: 122; the CDR1, CDR2, and CDR3 of SEQ ID NO: 126; the CDR1, CDR2, and CDR3 of SEQ ID NO: 130; the CDR1, CDR2, and CDR3 of SEQ ID NO: 134; the CDR1, CDR2, and CDR3 of SEQ ID NO: 142; the CDR1, CDR2, and CDR3 of SEQ ID NO: 146; the CDR1, CDR2, and CDR3 of SEQ ID NO: 150; the CDR1, CDR2, and CDR3 of SEQ ID NO: 154; the CDR1, CDR2, and CDR3 of SEQ ID NO: 158; the CDR1, CDR2, and CDR3 of SEQ ID NO: 162, the CDR1, CDR2, and CDR3 of SEQ ID NO: 166; the CDR1, CDR2, and CDR3 of SEQ ID NO: 170, the CDR1, CDR2, and CDR3 of SEQ ID NO: 174, the CDR1, CDR2, and CDR3 of SEQ ID NO: 178, the CDR1, CDR2, and CDR3 of SEQ ID NO: 182, or the CDR1, CDR2, and CDR3 of SEQ ID NO: 186.

In some embodiments, the molecule comprises SEQ ID NO: 51, SEQ ID NO: 52, SEQ ID NO: 53, and SEQ ID NO: 54. In some embodiments, the molecule comprises SEQ ID NO: 55, SEQ ID NO: 56, SEQ ID NO: 57, and SEQ ID NO: 58. In some embodiments, the molecule comprises SEQ ID NO: 67, SEQ ID NO: 68, SEQ ID NO: 69, and SEQ ID NO: 70. In some embodiments, the molecule comprises SEQ ID NO: 71, SEQ ID NO: 72, SEQ ID NO: 73, and SEQ ID NO: 74. In some embodiments, the molecule comprises SEQ ID NO: 75, SEQ ID NO: 76, SEQ ID NO: 77, and SEQ ID NO: 78. In some embodiments, the molecule comprises SEQ ID NO: 79, SEQ ID NO: 80, SEQ ID NO: 81, and SEQ ID NO: 82. In some embodiments, the molecule comprises SEQ ID NO: 83, SEQ ID NO: 84, SEQ ID NO: 85, and SEQ ID NO: 86. In some embodiments, the molecule comprises SEQ ID NO: 87, SEQ ID NO: 88, SEQ ID NO: 89, and SEQ ID NO: 90. In some embodiments, the molecule comprises SEQ ID NO: 91, SEQ ID NO: 92, SEQ ID NO: 93, and SEQ ID NO: 94. In some embodiments, the molecule comprises SEQ ID NO: 95, SEQ ID NO: 96, SEQ ID NO: 97, and SEQ ID NO: 98. In some embodiments, the molecule comprises SEQ ID NO: 99, SEQ ID NO: 100, SEQ ID NO: 101, and SEQ ID NO: 102. In some embodiments, the molecule comprises SEQ ID NO: 103, SEQ ID NO: 104, SEQ ID NO: 105, and SEQ ID NO: 106. In some embodiments, the molecule comprises SEQ ID NO: 107, SEQ ID NO: 108, SEQ ID NO: 109, and SEQ ID NO: 110. In some embodiments, the molecule comprises SEQ ID NO: 115, SEQ ID NO: 116, SEQ ID NO: 117, and SEQ ID NO: 118. In some embodiments, the molecule comprises SEQ ID NO: 119, SEQ ID NO: 120, SEQ ID NO: 121, and SEQ ID NO: 122.

In some embodiments, the molecule comprises SEQ ID NO: 123, SEQ ID NO: 124, SEQ ID NO: 125, and SEQ ID NO: 126. In some embodiments, the molecule comprises SEQ ID NO: 127, SEQ ID NO: 128, SEQ ID NO: 129, and SEQ ID NO: 130. In some embodiments, the molecule comprises SEQ ID NO: 131, SEQ ID NO: 132, SEQ ID NO: 133, and SEQ ID NO: 133. In some embodiments, the molecule comprises SEQ ID NO: 139, SEQ ID NO: 140, SEQ ID NO: 141, and SEQ ID NO: 142. In some embodiments, the molecule comprises SEQ ID NO: 143, SEQ ID NO: 144, SEQ ID NO: 145, and SEQ ID NO: 146. In some embodiments, the molecule comprises SEQ ID NO: 147, SEQ ID NO: 148, SEQ ID NO: 149, and SEQ ID NO: 150. In some embodiments, the molecule comprises SEQ ID NO: 151, SEQ ID NO: 152, SEQ ID NO: 153, and SEQ ID NO: 154. In some embodiments, the molecule comprises SEQ ID NO: 155, SEQ ID NO: 156, SEQ ID NO: 157, and SEQ ID NO: 158. In some embodiments, the molecule comprises SEQ ID NO: 159, SEQ ID NO: 160, SEQ ID NO: 161, and SEQ ID NO: 162. In some embodiments, the molecule comprises SEQ ID NO: 163, SEQ ID NO: 164, SEQ ID NO: 165, and SEQ ID NO: 166. In some embodiments, the molecule comprises SEQ ID NO: 167, SEQ ID NO: 168, SEQ ID NO: 169, and SEQ ID NO: 170. In some embodiments, the molecule comprises SEQ ID NO: 171, SEQ ID NO: 172, SEQ ID NO: 173, and SEQ ID NO: 174. In some embodiments, the molecule comprises SEQ ID NO: 175, SEQ ID NO: 176, SEQ ID NO: 177, and SEQ ID NO: 178. In some embodiments, the molecule comprises SEQ ID NO: 179, SEQ ID NO: 180, SEQ ID NO: 181, and SEQ ID NO: 182. In some embodiments, the molecule comprises SEQ ID NO: 183, SEQ ID NO: 184, SEQ ID NO: 185, and SEQ ID NO: 186.

In some embodiments, the molecule further comprises one or more disulfide bonds. In some embodiments, the molecule further comprises one or more linker sequences. In some embodiments, the molecule is capable of neutralizing a coronavirus. In some embodiments, the coronavirus is a SARS-CoV-2 virus. In some embodiments, the SARS-CoV-2 virus strain is selected from the group consisting of WA1, B.1.1.7, B1.526, B1.351, P1, B1.352, and B.1.427.

In some embodiments, the molecule specifically recognizes a first epitope and a second epitope on a SARS-CoV-2 particle. In some embodiments, the first epitope comprises at least a portion of a receptor binding domain (RBD) on a spike protein of a SARS-CoV-2 particle. In some embodiments, the second epitope comprises at least a portion of a N-terminal domain (NTD) on a spike protein of a SARS-CoV-2 particle.

In certain aspects, the invention provides a method of treating or preventing a COVID-19 infection in a subject in need thereof, the method comprising administering to the subject a composition comprising a bispecific molecule comprising a first polypeptide chain and a second polypeptide chain, wherein: the first polypeptide chain comprises: a first light chain comprising a variable domain and a constant domain; a first heavy chain comprising a variable domain and a constant domain; the second polypeptide chain comprises: a second light chain comprising a variable domain and a constant domain; and a second heavy chain comprising a variable domain and a constant domain.

In some embodiments, the first polypeptide chain and the second polypeptide chain are covalently bonded to one another. In some embodiments, the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 51, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 55, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 67, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 71, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 75, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 79, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 83, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 87, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 91, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 95, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 99, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 103, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 107, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 115, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 119, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 123, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 127, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 131, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 139, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 143, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 147, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 151, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 155, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 159, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 163, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 167, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 171, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 175, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 179, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO:183.

In some embodiments, the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 52, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 56, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 68; a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 72, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 76, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 80, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 84, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 88, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 92, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 96, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 100, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 104, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 108, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 116, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 120, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 124, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 128, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 132, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 140, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 144, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 148, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 152, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 156, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 160, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 164, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 168, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 172, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 176, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 180, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO:184.

In some embodiments, the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 53, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 57, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 69, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 73, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 77, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 81, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 85, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 89, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 93, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 97, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 101, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 105, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 109, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 117, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 121, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 125, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 129, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 133, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 141, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 145, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 149, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 153, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 157, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 161, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 165, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 169, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 173, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 177, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 181, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 185.

In some embodiments, the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 54, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 58, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 70, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 74, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 78, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 82, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 86, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 90, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 94, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 98, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 102, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 106, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 110, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 118, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 122, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 126, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 130, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 134, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 142, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 146, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 150, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 154, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 158, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 162, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 166, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 170, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 174, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 178, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 182, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 186.

In some embodiments, the variable domain of the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 41.

In some embodiments, the variable domain of the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 42.

In some embodiments, the variable domain of the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 29, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 23, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 39, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9.

In some embodiments, variable domain of the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 30, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 24, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 40, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10.

In some embodiments, the variable domain of the first heavy chain comprises the CDR1, CDR2, and CDR3 of SEQ ID NO: 51; the CDR1, CDR2, and CDR3 of SEQ ID NO: 55; the CDR1, CDR2, and CDR3 of SEQ ID NO: 67; the CDR1, CDR2, and CDR3 of SEQ ID NO: 71; the CDR1, CDR2, and CDR3 of SEQ ID NO: 75; the CDR1, CDR2, and CDR3 of SEQ ID NO: 79; the CDR1, CDR2, and CDR3 of SEQ ID NO: 83; the CDR1, CDR2, and CDR3 of SEQ ID NO: 87; the CDR1, CDR2, and CDR3 of SEQ ID NO: 91; the CDR1, CDR2, and CDR3 of SEQ ID NO: 95; the CDR1, CDR2, and CDR3 of SEQ ID NO: 99; the CDR1, CDR2, and CDR3 of SEQ ID NO: 103; the CDR1, CDR2, and CDR3 of SEQ ID NO: 107; the CDR1, CDR2, and CDR3 of SEQ ID NO: 115; the CDR1, CDR2, and CDR3 of SEQ ID NO: 119, the CDR1, CDR2, and CDR3 of SEQ ID NO: 123, the CDR1, CDR2, and CDR3 of SEQ ID NO: 127; the CDR1, CDR2, and CDR3 of SEQ ID NO: 131; the CDR1, CDR2, and CDR3 of SEQ ID NO: 139; the CDR1, CDR2, and CDR3 of SEQ ID NO: 143; the CDR1, CDR2, and CDR3 of SEQ ID NO: 147; the CDR1, CDR2, and CDR3 of SEQ ID NO: 151; the CDR1, CDR2, and CDR3 of SEQ ID NO: 155; the CDR1, CDR2, and CDR3 of SEQ ID NO: 159; the CDR1, CDR2, and CDR3 of SEQ ID NO: 163; the CDR1, CDR2, and CDR3 of SEQ ID NO: 167, the CDR1, CDR2, and CDR3 of SEQ ID NO: 171, the CDR1, CDR2, and CDR3 of SEQ ID NO: 175, the CDR1, CDR2, and CDR3 of SEQ ID NO: 179, or the CDR1, CDR2, and CDR3 of SEQ ID NO: 183.

In some embodiments, the variable domain of the first light chain comprises the CDR1, CDR2, and CDR3 of SEQ ID NO: 52; the CDR1, CDR2, and CDR3 of SEQ ID NO: 56; the CDR1, CDR2, and CDR3 of SEQ ID NO: 68; the CDR1, CDR2, and CDR3 of SEQ ID NO: 72; the CDR1, CDR2, and CDR3 of SEQ ID NO: 76; the CDR1, CDR2, and CDR3 of SEQ ID NO: 80; the CDR1, CDR2, and CDR3 of SEQ ID NO: 84; the CDR1, CDR2, and CDR3 of SEQ ID NO: 88; the CDR1, CDR2, and CDR3 of SEQ ID NO: 92; the CDR1, CDR2, and CDR3 of SEQ ID NO: 96; the CDR1, CDR2, and CDR3 of SEQ ID NO: 100; the CDR1, CDR2, and CDR3 of SEQ ID NO: 104; the CDR1, CDR2, and CDR3 of SEQ ID NO: 108; the CDR1, CDR2, and CDR3 of SEQ ID NO: 116; the CDR1, CDR2, and CDR3 of SEQ ID NO: 120; the CDR1, CDR2, and CDR3 of SEQ ID NO: 124; the CDR1, CDR2, and CDR3 of SEQ ID NO: 128; the CDR1, CDR2, and CDR3 of SEQ ID NO: 132; the CDR1, CDR2, and CDR3 of SEQ ID NO: 140; the CDR1, CDR2, and CDR3 of SEQ ID NO: 144; the CDR1, CDR2, and CDR3 of SEQ ID NO: 148; the CDR1, CDR2, and CDR3 of SEQ ID NO: 152; the CDR1, CDR2, and CDR3 of SEQ ID NO: 156; the CDR1, CDR2, and CDR3 of SEQ ID NO: 160; the CDR1, CDR2, and CDR3 of SEQ ID NO: 164; the CDR1, CDR2, and CDR3 of SEQ ID NO: 168, the CDR1, CDR2, and CDR3 of SEQ ID NO: 172, the CDR1, CDR2, and CDR3 of SEQ ID NO: 176, the CDR1, CDR2, and CDR3 of SEQ ID NO: 180, or the CDR1, CDR2, and CDR3 of SEQ ID NO: 184.

In some embodiments, the variable domain of the second heavy chain comprises the CDR1, CDR2, and CDR3 of SEQ ID NO: 53; the CDR1, CDR2, and CDR3 of SEQ ID NO: 57; the CDR1, CDR2, and CDR3 of SEQ ID NO: 69; the CDR1, CDR2, and CDR3 of SEQ ID NO: 73; the CDR1, CDR2, and CDR3 of SEQ ID NO: 77; the CDR1, CDR2, and CDR3 of SEQ ID NO: 81; the CDR1, CDR2, and CDR3 of SEQ ID NO: 85; the CDR1, CDR2, and CDR3 of SEQ ID NO: 89; the CDR1, CDR2, and CDR3 of SEQ ID NO: 93; the CDR1, CDR2, and CDR3 of SEQ ID NO: 97; the CDR1, CDR2, and CDR3 of SEQ ID NO: 101; the CDR1, CDR2, and CDR3 of SEQ ID NO: 105; the CDR1, CDR2, and CDR3 of SEQ ID NO: 109; the CDR1, CDR2, and CDR3 of SEQ ID NO: 117; the CDR1, CDR2, and CDR3 of SEQ ID NO: 121; the CDR1, CDR2, and CDR3 of SEQ ID NO: 125; the CDR1, CDR2, and CDR3 of SEQ ID NO: 129; the CDR1, CDR2, and CDR3 of SEQ ID NO: 133; the CDR1, CDR2, and CDR3 of SEQ ID NO: 141; the CDR1, CDR2, and CDR3 of SEQ ID NO: 145; the CDR1, CDR2, and CDR3 of SEQ ID NO: 149; the CDR1, CDR2, and CDR3 of SEQ ID NO: 153; the CDR1, CDR2, and CDR3 of SEQ ID NO: 157; the CDR1, CDR2, and CDR3 of SEQ ID NO: 161; the CDR1, CDR2, and CDR3 of SEQ ID NO: 165; the CDR1, CDR2, and CDR3 of SEQ ID NO: 169, the CDR1, CDR2, and CDR3 of SEQ ID NO: 173, the CDR1, CDR2, and CDR3 of SEQ ID NO: 177, the CDR1, CDR2, and CDR3 of SEQ ID NO: 181, or the CDR1, CDR2, and CDR3 of SEQ ID NO: 185.

In some embodiments, the variable domain of the second light chain comprises the CDR1, CDR2, and CDR3 of SEQ ID NO: 54; the CDR1, CDR2, and CDR3 of SEQ ID NO: 58; the CDR1, CDR2, and CDR3 of SEQ ID NO: 70; the CDR1, CDR2, and CDR3 of SEQ ID NO: 74; the CDR1, CDR2, and CDR3 of SEQ ID NO: 78; the CDR1, CDR2, and CDR3 of SEQ ID NO: 82; the CDR1, CDR2, and CDR3 of SEQ ID NO: 86; the CDR1, CDR2, and CDR3 of SEQ ID NO: 90; the CDR1, CDR2, and CDR3 of SEQ ID NO: 94; the CDR1, CDR2, and CDR3 of SEQ ID NO: 98; the CDR1, CDR2, and CDR3 of SEQ ID NO: 102; the CDR1, CDR2, and CDR3 of SEQ ID NO: 106; the CDR1, CDR2, and CDR3 of SEQ ID NO: 110; the CDR1, CDR2, and CDR3 of SEQ ID NO: 118; the CDR1, CDR2, and CDR3 of SEQ ID NO: 122; the CDR1, CDR2, and CDR3 of SEQ ID NO: 126; the CDR1, CDR2, and CDR3 of SEQ ID NO: 130; the CDR1, CDR2, and CDR3 of SEQ ID NO: 134; the CDR1, CDR2, and CDR3 of SEQ ID NO: 142; the CDR1, CDR2, and CDR3 of SEQ ID NO: 146; the CDR1, CDR2, and CDR3 of SEQ ID NO: 150; the CDR1, CDR2, and CDR3 of SEQ ID NO: 154; the CDR1, CDR2, and CDR3 of SEQ ID NO: 158; the CDR1, CDR2, and CDR3 of SEQ ID NO: 162, the CDR1, CDR2, and CDR3 of SEQ ID NO: 166; the CDR1, CDR2, and CDR3 of SEQ ID NO: 170, the CDR1, CDR2, and CDR3 of SEQ ID NO: 174, the CDR1, CDR2, and CDR3 of SEQ ID NO: 178, the CDR1, CDR2, and CDR3 of SEQ ID NO: 182, or the CDR1, CDR2, and CDR3 of SEQ ID NO: 186.

In some embodiments, the molecule comprises SEQ ID NO: 51, SEQ ID NO: 52, SEQ ID NO: 53, and SEQ ID NO: 54. In some embodiments, the molecule comprises SEQ ID NO: 55, SEQ ID NO: 56, SEQ ID NO: 57, and SEQ ID NO: 58. In some embodiments, the molecule comprises SEQ ID NO: 67, SEQ ID NO: 68, SEQ ID NO: 69, and SEQ ID NO: 70. In some embodiments, the molecule comprises SEQ ID NO: 71, SEQ ID NO: 72, SEQ ID NO: 73, and SEQ ID NO: 74. In some embodiments, the molecule comprises SEQ ID NO: 75, SEQ ID NO: 76, SEQ ID NO: 77, and SEQ ID NO: 78. In some embodiments, the molecule comprises SEQ ID NO: 79, SEQ ID NO: 80, SEQ ID NO: 81, and SEQ ID NO: 82. In some embodiments, the molecule comprises SEQ ID NO: 83, SEQ ID NO: 84, SEQ ID NO: 85, and SEQ ID NO: 86. In some embodiments, the molecule comprises SEQ ID NO: 87, SEQ ID NO: 88, SEQ ID NO: 89, and SEQ ID NO: 90. In some embodiments, the molecule comprises SEQ ID NO: 91, SEQ ID NO: 92, SEQ ID NO: 93, and SEQ ID NO: 94. In some embodiments, the molecule comprises SEQ ID NO: 95, SEQ ID NO: 96, SEQ ID NO: 97, and SEQ ID NO: 98. In some embodiments, the molecule comprises SEQ ID NO: 99, SEQ ID NO: 100, SEQ ID NO: 101, and SEQ ID NO: 102. In some embodiments, the molecule comprises SEQ ID NO: 103, SEQ ID NO: 104, SEQ ID NO: 105, and SEQ ID NO: 106. In some embodiments, the molecule comprises SEQ ID NO: 107, SEQ ID NO: 108, SEQ ID NO: 109, and SEQ ID NO: 110. In some embodiments, the molecule comprises SEQ ID NO: 115, SEQ ID NO: 116, SEQ ID NO: 117, and SEQ ID NO: 118.

In some embodiments, the molecule comprises SEQ ID NO: 119, SEQ ID NO: 120, SEQ ID NO: 121, and SEQ ID NO: 122. In some embodiments, the molecule comprises SEQ ID NO: 123, SEQ ID NO: 124, SEQ ID NO: 125, and SEQ ID NO: 126. In some embodiments, the molecule comprises SEQ ID NO: 127, SEQ ID NO: 128, SEQ ID NO: 129, and SEQ ID NO: 130. In some embodiments, the molecule comprises SEQ ID NO: 131, SEQ ID NO: 132, SEQ ID NO: 133, and SEQ ID NO: 133. In some embodiments, the molecule comprises SEQ ID NO: 139, SEQ ID NO: 140, SEQ ID NO: 141, and SEQ ID NO: 142. In some embodiments, the molecule comprises SEQ ID NO: 143, SEQ ID NO: 144, SEQ ID NO: 145, and SEQ ID NO: 146. In some embodiments, the molecule comprises SEQ ID NO: 147, SEQ ID NO: 148, SEQ ID NO: 149, and SEQ ID NO: 150. In some embodiments, the molecule comprises SEQ ID NO: 151, SEQ ID NO: 152, SEQ ID NO: 153, and SEQ ID NO: 154. In some embodiments, the molecule comprises SEQ ID NO: 155, SEQ ID NO: 156, SEQ ID NO: 157, and SEQ ID NO: 158. In some embodiments, the molecule comprises SEQ ID NO: 159, SEQ ID NO: 160, SEQ ID NO: 161, and SEQ ID NO: 162. In some embodiments, the molecule comprises SEQ ID NO: 163, SEQ ID NO: 164, SEQ ID NO: 165, and SEQ ID NO: 166. In some embodiments, the molecule comprises SEQ ID NO: 167, SEQ ID NO: 168, SEQ ID NO: 169, and SEQ ID NO: 170. In some embodiments, the molecule comprises SEQ ID NO: 171, SEQ ID NO: 172, SEQ ID NO: 173, and SEQ ID NO: 174. In some embodiments, the molecule comprises SEQ ID NO: 175, SEQ ID NO: 176, SEQ ID NO: 177, and SEQ ID NO: 178. In some embodiments, the molecule comprises SEQ ID NO: 179, SEQ ID NO: 180, SEQ ID NO: 181, and SEQ ID NO: 182. In some embodiments, the molecule comprises SEQ ID NO: 183, SEQ ID NO: 184, SEQ ID NO: 185, and SEQ ID NO: 186.

In some embodiments, the molecule further comprises one or more disulfide bonds. In some embodiments, the molecule further comprises one or more linker sequences. In some embodiments, the molecule is capable of neutralizing a coronavirus. In some embodiments, the coronavirus is a SARS-CoV-2 virus. In some embodiments, the SARS-CoV-2 virus strain is selected from the group consisting of WA1, B.1.1.7, B1.526, B1.351, P1, B1.352, and B.1.427. In some embodiments, the molecule specifically recognizes a first epitope and a second epitope on a SARS-CoV-2 particle. In some embodiments, the first epitope comprises at least a portion of a receptor binding domain (RBD) on a spike protein of a SARS-CoV-2 particle. In some embodiments, the second epitope comprises at least a portion of a N-terminal domain (NTD) on a spike protein of a SARS-CoV-2 particle. In some embodiments, the composition is administered in combination with a second therapeutic agent.

In certain aspects, the invention provides a synthesized monoclonal antibody, or a functional fragment thereof, comprising a heavy chain having a variable domain and a constant domain and a light chain having a variable domain and a constant domain, wherein the antibody binds an antigen on a coronavirus particle.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 41. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 23. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 42. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 2.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 24. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 41 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 42. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 2. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 23 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 24. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28.

In some embodiments, the heavy chain variable domain comprises SEQ ID NO: 5 and the light chain variable domain comprises SEQ ID NO: 6. In some embodiments, the heavy chain variable domain comprises SEQ ID NO: 41 and the light chain variable domain comprises SEQ ID NO: 42. In some embodiments, the heavy chain variable domain comprises SEQ ID NO: 35 and the light chain variable domain comprises SEQ ID NO: 36. In some embodiments, the heavy chain variable domain comprises SEQ ID NO: 1 and the light chain variable domain comprises SEQ ID NO: 2. In some embodiments, the heavy chain variable domain comprises SEQ ID NO: 23 and the light chain variable domain comprises SEQ ID NO: 24. In some embodiments, the heavy chain variable domain comprises SEQ ID NO: 13 and the light chain variable domain comprises SEQ ID NO: 14. In some embodiments, the heavy chain variable domain comprises SEQ ID NO: 27 and the light chain variable domain comprises SEQ ID NO: 28. In some embodiments, the heavy chain variable domain comprises the CDR1, CDR2, and CDR3 regions of SEQ ID NO: 5. In some embodiments, the heavy chain variable domain comprises the CDR1, CDR2, and CDR3 regions of SEQ ID NO: 41. In some embodiments, the heavy chain variable domain comprises the CDR1, CDR2, and CDR3 regions of SEQ ID NO: 1.

In some embodiments, the heavy chain variable domain comprises the CDR1, CDR2, and CDR3 regions of SEQ ID NO: 23. In some embodiments, the heavy chain variable domain comprises the CDR1, CDR2, and CDR3 regions of SEQ ID NO: 13. In some embodiments, the heavy chain variable domain comprises the CDR1, CDR2, and CDR3 regions of SEQ ID NO: 27.

In some embodiments, the light chain variable domain comprises the CDR1, CDR2, and CDR3 regions of SEQ ID NO: 6. In some embodiments, the light chain variable domain comprises the CDR1, CDR2, and CDR3 regions of SEQ ID NO: 42. In some embodiments, the light chain variable domain comprises the CDR1, CDR2, and CDR3 regions of SEQ ID NO: 36. In some embodiments, the light chain variable domain comprises the CDR1, CDR2, and CDR3 regions of SEQ ID NO: 2. In some embodiments, the light chain variable domain comprises the CDR1, CDR2, and CDR3 regions of SEQ ID NO: 24. In some embodiments, the light chain variable domain comprises the CDR1, CDR2, and CDR3 regions of SEQ ID NO: 14. In some embodiments, the light chain variable domain comprises the CDR1, CDR2, and CDR3 regions of SEQ ID NO: 28. In some embodiments, the heavy chain variable domain comprises the CDR1, CDR2, and CDR3 regions of SEQ ID NO: 5 and the light chain variable domain comprises the CDR1, CDR2, and CDR3 regions of SEQ ID NO: 6. In some embodiments, the heavy chain variable domain comprises the CDR1, CDR2, and CDR3 regions of SEQ ID NO: 41 and the light chain variable domain comprises the CDR1, CDR2, and CDR3 regions of SEQ ID NO: 42. In some embodiments, the heavy chain variable domain comprises the CDR1, CDR2, and CDR3 regions of SEQ ID NO: 35 and the light chain variable domain comprises the CDR1, CDR2, and CDR3 regions of SEQ ID NO: 36. In some embodiments, the heavy chain variable domain comprises the CDR1, CDR2, and CDR3 regions of SEQ ID NO: 1 and the light chain variable domain comprises the CDR1, CDR2, and CDR3 regions of SEQ ID NO: 2.

In some embodiments, the heavy chain variable domain comprises the CDR1, CDR2, and CDR3 regions of SEQ ID NO: 23 and the light chain variable domain comprises the CDR1, CDR2, and CDR3 regions of SEQ ID NO: 24. In some embodiments, the heavy chain variable domain comprises the CDR1, CDR2, and CDR3 regions of SEQ ID NO: 13 and the light chain variable domain comprises the CDR1, CDR2, and CDR3 regions of SEQ ID NO: 14. In some embodiments, the heavy chain variable domain comprises the CDR1, CDR2, and CDR3 regions of SEQ ID NO: 27 and the light chain variable domain comprises the CDR1, CDR2, and CDR3 regions of SEQ ID NO: 28.

In some embodiments, the antibody neutralizes a coronavirus. In some embodiments, the coronavirus is a SARS-CoV-2 virus. In some embodiments, the coronavirus is a SARS-CoV-2 variant strain. In some embodiments, the variant strain is selected from the group consisting of WA1, B.1.1.7, B1.526, B1.351, P1, B1.352, and B.1.427. In some embodiments, the antibody specifically binds an epitope on the surface of the coronavirus. In some embodiments, the epitope comprises at least a portion of a coronavirus spike protein. In some embodiments, the at least a portion of a coronavirus spike protein comprises a receptor binding domain (RBD) of a spike protein. In some embodiments, the at least a portion of a coronavirus spike protein comprises a N-Terminal domain (NTD) of a spike protein. In some embodiments, the coronavirus is a SARS-CoV-2 coronavirus.

In certain aspects, the invention provides a method of treating or preventing a COVID-19 infection in a subject in need thereof, the method comprising administering to the subject a therapeutically effective amount of a synthesized monoclonal antibody, or a functional fragment thereof, comprising a heavy chain having a variable domain and a constant domain and a light chain having a variable domain and a constant domain, wherein the antibody binds an antigen on a coronavirus particle.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 41. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 23. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6.

In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 42. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 2. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 24. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14. In some embodiments, the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 41 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 42. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 2. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 23 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 24.

In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14. In some embodiments, the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 27 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 28.

In some embodiments, the heavy chain variable domain comprises SEQ ID NO: 5 and the light chain variable domain comprises SEQ ID NO: 6. In some embodiments, the heavy chain variable domain comprises SEQ ID NO: 41 and the light chain variable domain comprises SEQ ID NO: 42. In some embodiments, the heavy chain variable domain comprises SEQ ID NO: 35 and the light chain variable domain comprises SEQ ID NO: 36. In some embodiments, the heavy chain variable domain comprises SEQ ID NO: 1 and the light chain variable domain comprises SEQ ID NO: 2. In some embodiments, the heavy chain variable domain comprises SEQ ID NO: 23 and the light chain variable domain comprises SEQ ID NO: 24. In some embodiments, the heavy chain variable domain comprises SEQ ID NO: 13 and the light chain variable domain comprises SEQ ID NO: 14.

In some embodiments, the heavy chain variable domain comprises SEQ ID NO: 27 and the light chain variable domain comprises SEQ ID NO: 28. In some embodiments, the heavy chain variable domain comprises the CDR1, CDR2, and CDR3 regions of SEQ ID NO: 5. In some embodiments, the heavy chain variable domain comprises the CDR1, CDR2, and CDR3 regions of SEQ ID NO: 41. In some embodiments, the heavy chain variable domain comprises the CDR1, CDR2, and CDR3 regions of SEQ ID NO: 1. In some embodiments, the heavy chain variable domain comprises the CDR1, CDR2, and CDR3 regions of SEQ ID NO: 23. In some embodiments, the heavy chain variable domain comprises the CDR1, CDR2, and CDR3 regions of SEQ ID NO: 13. In some embodiments, the heavy chain variable domain comprises the CDR1, CDR2, and CDR3 regions of SEQ ID NO: 27. In some embodiments, the light chain variable domain comprises the CDR1, CDR2, and CDR3 regions of SEQ ID NO: 6. In some embodiments, the light chain variable domain comprises the CDR1, CDR2, and CDR3 regions of SEQ ID NO: 42. In some embodiments, the light chain variable domain comprises the CDR1, CDR2, and CDR3 regions of SEQ ID NO: 36. In some embodiments, the light chain variable domain comprises the CDR1, CDR2, and CDR3 regions of SEQ ID NO: 2. In some embodiments, the light chain variable domain comprises the CDR1, CDR2, and CDR3 regions of SEQ ID NO: 24.

In some embodiments, the light chain variable domain comprises the CDR1, CDR2, and CDR3 regions of SEQ ID NO: 14. In some embodiments, the light chain variable domain comprises the CDR1, CDR2, and CDR3 regions of SEQ ID NO: 28. In some embodiments, the heavy chain variable domain comprises the CDR1, CDR2, and CDR3 regions of SEQ ID NO: 5 and the light chain variable domain comprises the CDR1, CDR2, and CDR3 regions of SEQ ID NO: 6. In some embodiments, the heavy chain variable domain comprises the CDR1, CDR2, and CDR3 regions of SEQ ID NO: 41 and the light chain variable domain comprises the CDR1, CDR2, and CDR3 regions of SEQ ID NO: 42. In some embodiments, the heavy chain variable domain comprises the CDR1, CDR2, and CDR3 regions of SEQ ID NO: 35 and the light chain variable domain comprises the CDR1, CDR2, and CDR3 regions of SEQ ID NO: 36. In some embodiments, the heavy chain variable domain comprises the CDR1, CDR2, and CDR3 regions of SEQ ID NO: 1 and the light chain variable domain comprises the CDR1, CDR2, and CDR3 regions of SEQ ID NO: 2. In some embodiments, the heavy chain variable domain comprises the CDR1, CDR2, and CDR3 regions of SEQ ID NO: 23 and the light chain variable domain comprises the CDR1, CDR2, and CDR3 regions of SEQ ID NO: 24. In some embodiments, the heavy chain variable domain comprises the CDR1, CDR2, and CDR3 regions of SEQ ID NO: 13 and the light chain variable domain comprises the CDR1, CDR2, and CDR3 regions of SEQ ID NO: 14. In some embodiments, the heavy chain variable domain comprises the CDR1, CDR2, and CDR3 regions of SEQ ID NO: 27 and the light chain variable domain comprises the CDR1, CDR2, and CDR3 regions of SEQ ID NO: 28.

In some embodiments, the antibody neutralizes a coronavirus. In some embodiments, the coronavirus is a SARS-CoV-2 virus. In some embodiments, the coronavirus is a SARS-CoV-2 variant strain. In some embodiments, the variant strain is selected from the group consisting of WA1, B.1.1.7, B1.526, B1.351, P1, B1.352, and B.1.427. In some embodiments, the antibody specifically binds an epitope on the surface of the coronavirus. In some embodiments, the epitope comprises at least a portion of a coronavirus spike protein. In some embodiments, the at least a portion of a coronavirus spike protein comprises a receptor binding domain (RBD) of a spike protein. In some embodiments, the at least a portion of a coronavirus spike protein comprises a N-Terminal domain (NTD) of a spike protein. In some embodiments, the coronavirus is a SARS-CoV-2 coronavirus. In some embodiments, the antibody is administered in combination with a second pharmaceutical agent.

In certain aspects, the invention provides a bispecific molecule comprising a first polypeptide chain and a second polypeptide chain, wherein: the first polypeptide chain comprises: a first light chain comprising a variable domain and a constant domain; a first heavy chain comprising a variable domain and a constant domain; the second polypeptide chain comprises: a second light chain comprising a variable domain and a constant domain; and a second heavy chain comprising a variable domain and a constant domain. In some embodiments, the first polypeptide chain and the second polypeptide chain are covalently bonded to one another.

In some embodiments, the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 47, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 111, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 135, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 187, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 211. In some embodiments, the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 48, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 112, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 136, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 188, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 212.

In some embodiments, the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 49, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 113, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 137, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 189, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 213. In some embodiments, the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 50, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 114, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 138, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 190, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 214. In some embodiments, the variable domain of the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13.

In some embodiments, the variable domain of the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14. In some embodiments, the variable domain of the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 45, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43. In some embodiments, the variable domain of the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 46, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44.

In some embodiments, the variable domain of the first heavy chain comprises CDR1, CDR2, and CDR3 of SEQ ID NO: 47; CDR1, CDR2, and CDR3 of SEQ ID NO: 111; CDR1, CDR2, and CDR3 of SEQ ID NO: 135; CDR1, CDR2, and CDR3 of SEQ ID NO: 187; or CDR1, CDR2, and CDR3 of SEQ ID NO: 211. In some embodiments, the variable domain of the first light chain comprises CDR1, CDR2, and CDR3 of SEQ ID NO: 48, CDR1, CDR2, and CDR3 of SEQ ID NO: 112; CDR1, CDR2, and CDR3 of SEQ ID NO: 136; CDR1, CDR2, and CDR3 of SEQ ID NO: 188; or CDR1, CDR2, and CDR3 of SEQ ID NO: 212. In some embodiments, the variable domain of the second heavy chain comprises CDR1, CDR2, and CDR3 of SEQ ID NO: 49, CDR1, CDR2, and CDR3 of SEQ ID NO: 113; CDR1, CDR2, and CDR3 of SEQ ID NO: 137; CDR1, CDR2, and CDR3 of SEQ ID NO: 189; or CDR1, CDR2, and CDR3 of SEQ ID NO: 213. In some embodiments, the variable domain of the second light chain comprises CDR1, CDR2, and CDR3 SEQ ID NO: 50, CDR1, CDR2, and CDR3 of SEQ ID NO: 114; CDR1, CDR2, and CDR3 of SEQ ID NO: 138; CDR1, CDR2, and CDR3 of SEQ ID NO: 190; or CDR1, CDR2, and CDR3 of SEQ ID NO: 214.

In some embodiments, the molecule comprises SEQ ID NO: 47, SEQ ID NO: 48, SEQ ID NO: 49, and SEQ ID NO: 50. In some embodiments, the molecule comprises SEQ ID NO:111, SEQ ID NO: 112, SEQ ID NO: 113, and SEQ ID NO: 114. In some embodiments, the molecule comprises SEQ ID NO: 135, SEQ ID NO: 135, SEQ ID NO: 137, and SEQ ID NO: 138. In some embodiments, the molecule comprises SEQ ID NO: 187, SEQ ID NO: 188, SEQ ID NO: 189, and SEQ ID NO: 190. In some embodiments, the molecule comprises SEQ ID NO: 211, SEQ ID NO: 212, SEQ ID NO: 213, and SEQ ID NO: 214.

In some embodiments, the molecule further comprises one or more disulfide bonds. In some embodiments, the molecule further comprises one or more linker sequences. In some embodiments, the molecule is capable of neutralizing a coronavirus. In some embodiments, the coronavirus is a SARS-CoV-2 virus. In some embodiments, the SARS-CoV-2 virus strain is selected from the group consisting of WA1, B.1.1.7, B1.526, B1.351, P1, B1.352, and B.1.427. In some embodiments, the molecule specifically recognizes a first epitope and a second epitope on a SARS-CoV-2 particle. In some embodiments, the first epitope comprises at least a portion of a receptor binding domain (RBD) on a spike protein of a SARS-CoV-2 particle. In some embodiments, the second epitope comprises at least a portion of a N-terminal domain (NTD) on a spike protein of a SARS-CoV-2 particle.

In certain aspects, the invention provides a method of treating or preventing a COVID-19 infection in a subject in need thereof, the method comprising administering to the subject a composition comprising a bispecific molecule comprising a first polypeptide chain and a second polypeptide chain, wherein: the first polypeptide chain comprises: a first light chain comprising a variable domain and a constant domain; a first heavy chain comprising a variable domain and a constant domain; the second polypeptide chain comprises: a second light chain comprising a variable domain and a constant domain; and a second heavy chain comprising a variable domain and a constant domain. In some embodiments, the first polypeptide chain and the second polypeptide chain are covalently bonded to one another.

In some embodiments, the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 47, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 111, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 135, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 187, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 211.

In some embodiments, the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 48, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 112, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 136, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 188, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 212.

In some embodiments, the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 49, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 113, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 137, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 189, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 213.

In some embodiments, the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 50, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 114, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 138, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 190, or a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 214.

In some embodiments, the variable domain of the first heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 35, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13. In some embodiments, the variable domain of the first light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 36, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14. In some embodiments, the variable domain of the second heavy chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 45, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43.

In some embodiments, the variable domain of the second light chain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 46, a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44.

In some embodiments, the variable domain of the first heavy chain comprises CDR1, CDR2, and CDR3 of SEQ ID NO: 47; CDR1, CDR2, and CDR3 of SEQ ID NO: 111; CDR1, CDR2, and CDR3 of SEQ ID NO: 135; CDR1, CDR2, and CDR3 of SEQ ID NO: 187; or CDR1, CDR2, and CDR3 of SEQ ID NO: 211. In some embodiments, the variable domain of the first light chain comprises CDR1, CDR2, and CDR3 of SEQ ID NO: 48, CDR1, CDR2, and CDR3 of SEQ ID NO: 112; CDR1, CDR2, and CDR3 of SEQ ID NO: 136; CDR1, CDR2, and CDR3 of SEQ ID NO: 188; or CDR1, CDR2, and CDR3 of SEQ ID NO: 212. In some embodiments, the variable domain of the second heavy chain comprises CDR1, CDR2, and CDR3 of SEQ ID NO: 49, CDR1, CDR2, and CDR3 of SEQ ID NO: 113; CDR1, CDR2, and CDR3 of SEQ ID NO: 137; CDR1, CDR2, and CDR3 of SEQ ID NO: 189; or CDR1, CDR2, and CDR3 of SEQ ID NO: 213. In some embodiments, the variable domain of the second light chain comprises CDR1, CDR2, and CDR3 SEQ ID NO: 50, CDR1, CDR2, and CDR3 of SEQ ID NO: 114; CDR1, CDR2, and CDR3 of SEQ ID NO: 138; CDR1, CDR2, and CDR3 of SEQ ID NO: 190; or CDR1, CDR2, and CDR3 of SEQ ID NO: 214. In some embodiments, the molecule comprises SEQ ID NO: 47, SEQ ID NO: 48, SEQ ID NO: 49, and SEQ ID NO: 50. In some embodiments, the molecule comprises SEQ ID NO:111, SEQ ID NO: 112, SEQ ID NO: 113, and SEQ ID NO: 114. In some embodiments, the molecule comprises SEQ ID NO: 135, SEQ ID NO: 135, SEQ ID NO: 137, and SEQ ID NO: 138. In some embodiments, the molecule comprises SEQ ID NO: 187, SEQ ID NO: 188, SEQ ID NO: 189, and SEQ ID NO: 190. In some embodiments, the molecule comprises SEQ ID NO: 211, SEQ ID NO: 212, SEQ ID NO: 213, and SEQ ID NO: 214.

In some embodiments, the molecule further comprises one or more disulfide bonds. In some embodiments, the molecule further comprises one or more linker sequences. In some embodiments, the molecule is capable of neutralizing a coronavirus. In some embodiments, the coronavirus is a SARS-CoV-2 virus. In some embodiments, the SARS-CoV-2 virus strain is selected from the group consisting of WA1, B.1.1.7, B1.526, B1.351, P1, B1.352, and B.1.427.

In some embodiments, the molecule specifically recognizes a first epitope and a second epitope on a SARS-CoV-2 particle. In some embodiments, the first epitope comprises at least a portion of a receptor binding domain (RBD) on a spike protein of a SARS-CoV-2 particle. In some embodiments, the second epitope comprises at least a portion of a N-terminal domain (NTD) on a spike protein of a SARS-CoV-2 particle. In some embodiments, the composition is administered in combination with a second therapeutic agent.

Antibody Structure

Antibodies are glycoproteins with an immunoglobulin (Ig) monomeric functional domain. Secreted antibodies can have multiple Ig units. The Ig monomer is a “Y”-shaped molecule with four polypeptide chains: two identical heavy chains and two identical light chains. The chains can be connected by disulfide bonds.

There are five types of mammalian Ig heavy chains referred to as: α, δ, ε, γ, and μ, defining the class of antibody. Distinct heavy chains differ in their size and composition. Each heavy chain comprises of two regions—the constant region and the variable region. The constant region is identical in all antibodies of the same isotype, but differs in antibodies of different isotypes. The variable region of the heavy chain differs in antibodies produced by different B cells, but is the same for all antibodies produced by a single B cell or a B cell clone. The variable region of each heavy chain is approximately 110 amino acids long.

There are two types of light chains in mammals—lambda (λ) and kappa (κ). A light chain comprises of two domains, the constant domain and the variable domain. The approximate length of the light chain is 211 to 217 amino acids. Each antibody contains two identical light chains.

The two arms of the Y-shaped antibody molecule include the antibody binding sites, which that can bind two antigens. The two antigens can be identical or they can be different for example in bispecific antibodies. This region of the antibody is called the Fab (fragment, antigen binding) region and it confers the antibody with its specificity. Thus, the Fab includes one constant and one variable domain from each heavy and each light chain of the antibody. The variable domain, also called the Fv region, is the most important region for recognizing and binding to antigens such as viral proteins or receptors on the surface of host cells. There are three variable loops light chain (VL) and three variable loops on the heavy (VH) chain of the antibody, which are responsible for binding to the antigen. The loops are called the complementarity determining regions (CDRs).

The base of the Y-shaped molecule is called the Fc (Fragment, crystallizable) region and it is important for modulating immune cell activity. The Fc includes two heavy chains and it can bind to specific to ensure that each antibody generates an appropriate immune response for a particular antigen. It can also mediate different physiological effects including opsonization, cell lysis, and degranulation of mast cells, basophils and eosinophils. Additionally, the two N-terminal fragments of the antibody are called the Fab region, and the C-terminal fragment is called the Fc region.

In one embodiment, the subject matter described herein relates to monoclonal and bispecific antibodies, variants, fusion proteins comprising an antibody portion with an antigen recognition site of the required specificity. In some embodiments, antigen-binding fragment of an antibody is a portion of an antibody that possesses an at least one antigen recognition site. Fragments include for example but not limited to Fab, Fab′, F(ab′)2 Fv), and single chain (scFv).

The antibodies can be produced recombinantly by any means known in the art. In one embodiment, such an antibody is sequenced and the polynucleotide sequence is then cloned into a vector for expression or propagation. In one embodiment, the antibody is synthesized. The sequence encoding the antibody of interest may be maintained in a vector in a host cell and the

host cell can then be expanded and frozen for future use. The polynucleotide sequence of such antibodies may be used for genetic manipulation to generate the bispecific molecules described herein.

Antibody Therapy

Antibody therapy uses monoclonal or bispecific antibodies to bind to pre-determined antigens. This therapy can stimulate the patient's immune system to attack those antigens such as viruses. In the clinical setting, the most common route of administration of therapeutic antibodies is the intravenous (IV) infusion. The can be followed by subcutaneous administration, although an intramuscular injection is also possible. Oral administration routes of antibodies are also being developed. The monoclonal and bispecific antibodies described herein can be administered to a subject through any useful method known in the art. The monoclonal and bispecific antibodies described herein can be administered daily. The monoclonal and bispecific antibodies described herein can be administered twice, three times, four times, five times, or several times a day. The monoclonal and bispecific antibodies described herein can be administered for 1, 2, 3, 4, 5, 6 days or for 1 or 2 weeks. In some embodiments, the monoclonal and bispecific antibodies described herein can be administered for a period longer than two weeks. The monoclonal and bispecific antibodies described herein can be administered in combination with any other therapeutically effective agent or a combination of agents.

COVID-19 Therapeutics—mAbs

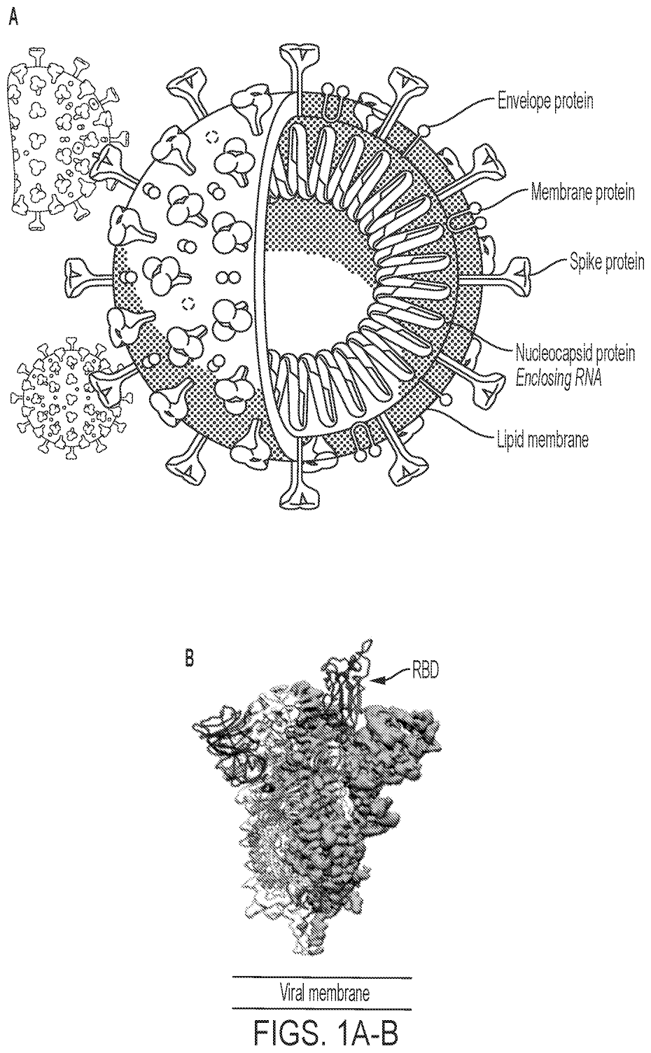

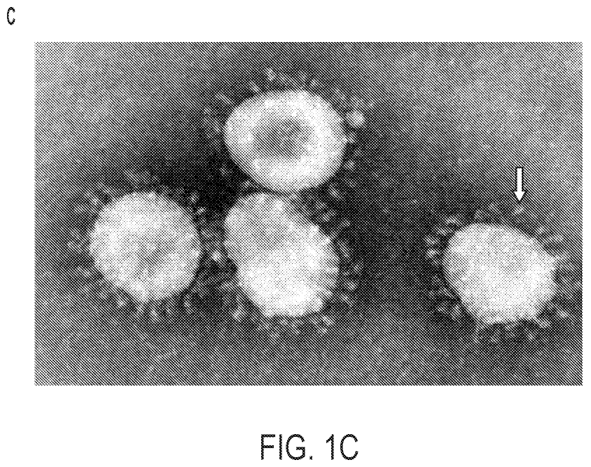

The only treatment currently approved for the treatment of patients with COVID-19 is remdesivir. Remdesivir shows some benefit in shortening the time for recovery from severe COVID-19. There is currently an effort in the field to identify biologics or small molecules that have more potent and broad effects against the spread of the SARS-CoV-2 virus. FIG. 1A shows a SARS-CoV-2 viral particle schematic diagram. FIG. 1B shows the spike protein trimer, the protein that located on the surface of the virus and binds to a cell receptor on the target cells. The RBD is a critical component of the viral spike glycoprotein that is found on coronaviruses including SARS-CoV-2 and plays the most important role in viral attachment, fusion and entry into the host cell. The receptor binding domain (RBD) is shown in green on top. The spike protein also has an N-terminal domain (NTD). RBD and NTD are targeted by antibodies against SARS-CoV-2 and variant strains. The spike protein trimer can be used to isolate B-cells from convalescent patient samples. FIG. 1C shows an electronic micrograph of coronavirus particles. The arrow denotes the viral spike protein on the viral membrane.

In some embodiments, the subject matter described herein relates to the isolation, characterization and providing the sequences of potent monoclonal antibodies (mAbs) against the SARS-CoV-2 virus, which causes COVID-19. In some embodiments, the subject matter disclosed herein describes the optimal COVID-19 patients from which to isolate potent mAbs against SARS-CoV-2. The mAbs can be isolated from blood samples from patients diagnosed with COVID-19 or patients exhibiting COVID-19 related symptoms. Once the antibody sequences are determined from these samples, the mAbs can be synthesized in vitro. In some embodiments, the mAb sequences can be modified and optimized prior to synthesis. These mAb can be used for subsequent characterization experiments. In some embodiment, the subject matter disclosed herein relates to the identification of mAbs significantly more potent, with than any other SARS-CoV-2 mAb known in the art to date. In some embodiments, the mAb lower IC50 and/or IC90 values,

The SARS-CoV-2 neutralizing mAbs described herein can be used for treatment of subjects infected with SARS-CoV-2. In some embodiments, the mAbs described herein reduce SARS-CoV-2 viral load in a subject in need thereof. In some embodiment, the mAbs described herein decrease disease severity in a subject in need thereof. In some embodiments, the mAbs described herein improve clinical outcome of COVID-19 in subjects in need thereof.

In some embodiments, the mAbs described herein can be used as prophylaxis to prevent high risk subjects and individuals from becoming infected with SARS-CoV-2. In some embodiment, the high risk subject can be healthcare workers in close proximity to COVID-19 patients or the family members of these healthcare workers. In some embodiment, the mAbs described herein can be used as prophylaxis for the general population at large.

Flow Through of Identification Process for the Potent COVID-19 mAbs

In one embodiment, the subject matter disclosed herein related to identification, isolation from patients, and characterization of potent mAbs against SARS-CoV-2, its variants, or any coronavirus. One embodiment of the method described herein for isolation of such antibodies is described below. In one embodiment, the subject matter disclosed herein related to administration of the antibodies described herein to subjects in need thereof to treat or prevent disease. In some embodiments, the treatment includes administration of one type of monoclonal antibody. In some embodiments, the treatment includes administration of a cocktail of two or more monoclonal antibodies.

Plasma samples from COVID-19 patients can be isolated and evaluated for the ability of potential antibodies contained within these plasma samples to: A) bind the viral envelope of SARS-CoV-2 or a variant strain (analysis can be by ELISA); and B) neutralize SARS-CoV-2 or a variant strain in vitro. Plasma samples from COVID-19 patients with severe disease or symptomology can have stronger antiviral activities than plasma samples from COVID-19 patients with non-severe disease. Plasma sample which are determined to have the strongest activity can be used to select patients for further analysis of monoclonal antibodies.

Peripheral blood mononuclear cells (PMBCs) can be isolated from COVID-19 patients whose plasma samples exhibit the strongest activity in order to isolate single B cells. These B-cells contain mAb-sequences that can be responsible for the strong plasma antiviral activity identified above. The antibody sequences can be recovered from these single B cells by any high throughput sequencing methods known in the art. The genes from the single B cells encoding for these mAb sequences can be synthesized and cloned into expression vectors. The encoded monoclonal antibodies can then be expressed in vitro and purified for subsequent analyses and characterization.

In vitro-produced and purified monoclonal antibodies can then tested for their ability to: A) bind to the virus envelope of SARS-CoV-2 or a variant strain (analysis can be by ELISA); and B) neutralize SARS-CoV-2 or a variant strain, thus stopping or preventing infections caused by coronaviruses. Neutralization analysis can be performed by both pseudotyped virus assay and/or live virus neutralization assays. Those mAbs that perform the best can then subjected to additional characterization studies including but not limited to ELISA competition assays, surface plasmon resonance (SPR) binding affinity analyses and size exclusion chromatography. Antibody performance can be indicated by potency, IC50 concentration, and IC90 concentration. An IC50 is a quantitative measure used to indicated the concentration of an antibody needed to inhibit a virus or a virus population by 50%. An IC90 is a quantitative measure used to indicated the concentration of an antibody needed to inhibit a virus or a virus population by 90%. These are measures of the potency of the antibody.

In one embodiment of the subject matter described herein, more than 100 million cells have been screened by the described method from 8 patients, 276 mAb sequences were synthesized, 66 mAbs were tested and characterized. In one embodiment, 6 mAbs have emerged as top hits with good antiviral activity against SARS-CoV-2. In one embodiment, 1 mAb has emerged with exceptional antiviral activity and potency against SARS-CoV-2. In one embodiment, the subject matter described herein relates to monoclonal antibodies with significantly higher potency than any other SARS-CoV-2 mAb known in the art to date. In one embodiment, the subject matter described herein relates to monoclonal antibodies with significantly lower IC50 and IC90 concentration values than any other mAb known in the art to date against SARS-CoV-2 or a variant strain.

In one embodiment, additional monoclonal antibodies are identified from the samples in the screening pipeline and if additional plasma samples from COVID-19 patient are screened for their ability to neutralize SARS-CoV-2.

The mAbs generated through the process described herein are significantly more potent than other antibodies known in the art against SARS-CoV-2 or a variant strain. In some embodiment, the subject matter described herein relates to mAbs against SARS-CoV-2 with high antiviral activity in subjects. In some embodiments the high antiviral activity is higher than the activity of SARS-CoV-2 known in the art. In some embodiments, the subjects are humans. In some embodiments, the subjects are human patients.

In some embodiments, more potent antibodies require smaller amounts (lower concentrations) to be administered to the subject in need thereof to achieve the desired COVID-19-related clinical effects. This can be achieved through administration of lower dosages to subjects. Therefore fewer potential side effects will be observed with less frequent dosing regimens or smaller amount of antibodies. This potentially will lower the antibody production costs. In some embodiments, the mAb against SARS-CoV-2 lead to faster recovery of subjects with COVID-19 and greater efficacy overall.

In some embodiments, the mAb described herein target multiple epitopes on the SARS-CoV-2 virus or a variant strain. In some embodiments, the subject matter disclosed herein relates to an antibody combination cocktails to be administered to the subject. In some embodiments, the antibodies in the cocktails target multiple epitopes on the SARS-CoV-2 virus or a variant strain. In some embodiments, the antibody combination cocktails provide even greater efficacy and lower the possibility of viral resistance than each individual antibody of the cocktail.

In some embodiments, the mAb described herein can be used for treatment and/or prevention of COVID-19 infection. In some embodiments, the mAbs described herein can be administered to the subject as a prevention or prophylaxis. In some embodiments, the mAbs can be used for diagnosis of COVID-19 subject exposure. In some embodiment, the mAbs described herein can be utilized in laboratory research and development activities.

Sequences of the mAbs described herein against SARS-CoV-2 or a variant strain

Underlined and italicized amino acids represent the respective complementarity-determining regions (CDRs). There are three CDRs per sequence, appearing on the sequence in the order CDR1, CDR2, and CDR3.

In one embodiment, the monoclonal antibody 2-4 comprises SEQ ID NOs: 1 and 2.

SEQ ID NO: 1 is the variable region of the heavy chain of antibody number 2-4.

QVQLVQSGAEVKKPGASVKVSCKASGYTFTGYYMHWVRQAPGQGLEWMGW
INPNSGGTNYTQMFQGRVTMTRDTSISTAYMEVSRLRSDDTAVYYCARDR
SWAVVYYYMDVWGKGTTVTVSS

SEQ ID NO: 2 is the variable region of the light chain of antibody number 2-4.

QSALTQPPSASGSPGQSVTISCTGTSSDVGGYNYVSWYQQHPGKAPKLMI
YEVSKRPSGVPDRFSGSKSGNTASLTVSGLQAEDEADYYCSSYAGSNNLV
FGGGTKLTVL

In one embodiment, the monoclonal antibody 4-8 comprises SEQ ID NOs: 3 and 4.

SEQ ID NO: 3 is the variable region of the heavy chain of antibody number 4-8.

QVQLVQSGAEVKKAGSSVKVSCKASGGTFSSHTITWVRQAPGQGLEWMGR
IIPILGIANYAQKFQGRVTITADKSTSTAYMELSSLRSEDTAVYYCASLQ
TVDTAIEKYYGMDVWGQGTTVTVSS

SEQ ID NO: 4 is the variable region of the light chain of antibody number 4-8

SYELTQPPSVSVSPGQTASITCSGDKLGDKYACWYQQKPGQSPVLVIYQD
NKRPSGIPERFSGSNSGNTATLTISGTQAMDEADYYCQAWDSSTAVFGGG
TKLTVL

In one embodiment, the monoclonal antibody 2-15 comprises SEQ ID NOs: 5 and 6.

SEQ ID NO: 5 is the variable region of the heavy chain of antibody number 2-15.

QVQLVQSGAEVKKPGASVRVSCKASGYTFTGYYMHWVRQAPGQGLEWMGW
INPISDGTNYAQKFQGWVTMTRDTSISTVYMELSRLRSDDTAVYYCARGG
SRCSGGNCYGWAYDAFDIWGQGTMITVSS

SEQ ID NO: 6 is the variable region of the light chain of antibody number 2-15.

QSALTQPASVSGSPGQSITISCTGTSSDVGGYNFVSWYQQHPGKAPKLMI
YDVSKRPSGVSNRFSGSKSGNTASLTISGLQAEDEADCYCSSYTSSSTFV
FGTGTKVTVL

In one embodiment, the monoclonal antibody 2-38 comprises SEQ ID NOs: 7 and 8.

SEQ ID NO: 7 is the variable region of the heavy chain of antibody number 2-38.

EVQLVESGGGLVKPGGSLRLSCAASGFTFSSYSMNWVRQAPGKGLEWVSS
ISSSSSYIYYADSVKGRFTISRDNAKNSLYLQMNSLRAEDTAVYYCTRAG
WELRLDAFDIWGQGTMVTVSS

SEQ ID NO: 8 is the variable region of the light chain of antibody number 2-38.

SSELTQDPAVSVALGQTVRITCQGDSLRSSYASWYQQKPGQAPILVIYDK
NNRPSGIPDRFSGSSSGNTASLTITGAQAEDEADYYCNSRDSSGILFGGG
TKLTVL

In one embodiment, the monoclonal antibody 2-51 comprises SEQ ID NOs: 9 and 10.

SEQ ID NO: 9 is the variable region of the heavy chain of antibody number 2-51.

QVQLVQSGAEVKKPGASVKVSCKVSGYTLIELSMHWVRQAPGKGLEWMGG
FDPEDVETIYAQQFQGRVTMTEDTSTDTAYMELSSLRSEDTAVYYCATGW
AYKSTWYFGYWGQGTLVTVSS

SEQ ID NO: 10 is the variable region of the light chain of antibody number 2-51.

QSALTQPPSASGSPGQSVTISCTGTSSDVGGYNYVSWYQQHPGKAPKLMI
YEVSKRPSGVPDRFSGSKSGNTASLTVSGLQAEDEADYYCSSYAGSRMGF
GGGTKLTVL

In one embodiment, the monoclonal antibody 4-32 comprises SEQ ID NOs: 11 and 12.

SEQ ID NO: 11 is the variable region of the heavy chain of antibody number 4-32.

EVQLVESGGGLVQPGRSLRLSCAASGFSFDDYAMHWVRQAPGKGLEWVSG
ISWNSGSIGYADSVKGRFTISRDNAKNSLYLQMNSLRAEDTALYYCAKGL
VQDYDSSGYFFADRVSAFDIWGQGTMVTVSS

SEQ ID NO: 12 is the variable region of the light chain of antibody number 4-32.

DIQMTQSPSSLSASVGDRVTITCRASQSINSYLNWYQQKPGKAPKLLIYA
ASSLQSGVPSRFSGSGSGTDFTLTISSLQPEDFATYYCQQSYSTPLTFGG
GTKVEIK

In one embodiment, the monoclonal antibody 2-7 comprises SEQ ID NOs: 13 and 14.

SEQ ID NO: 13 is the variable region of the heavy chain of antibody number 2-7, which specifically recognizes the receptor binding domain (RBD).

SQITLKESGPTLVKPTQTLTLTCTFSGFSLSTSGVGVGWIRQPPGKALEW
LALIYWDDDKRYSPSLKSRLTITKDTSKNQVVLTMTNMDPVDTATYYCAH
HKIERIFDYWGQGTLVTVSSAS

SEQ ID NO: 14 is the variable region of the light chain of antibody number 2-7, which specifically recognizes the RBD.

QSALAQPASVSGSPGQSITISCTGTSSDVGAYNYVSWYQQHPGKAPKLMI
YDVSKRPSGVSNRFSGSKSGNTASLTISGLQAEDEADYYCSSYTTSSTVF
GGGTKLTVL

In one embodiment, the monoclonal antibody 1-57 comprises SEQ ID NOs: 15 and 16.

SEQ ID NO: 15 is the variable region of the heavy chain of antibody number 1-57, which specifically recognizes the RBD.

EVHLVESGGGLVQPGGSLRLSCAASGFTFSDHYMDWVRQAPGKGLEWVGR
TRNKANSYTTEYAASVKGRFTISRDDAKNSLYLQMNSLKTEDTAVYYCAR
VHRWAYCINGVCFGAYSDYWGQGTLNYNSS

SEQ ID NO: 16 is the variable region of the light chain of antibody number 1-57, which specifically recognizes the RBD.

EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPSTFG
QGTKLEIK

In one embodiment, the monoclonal antibody 1-20 comprises SEQ ID NOs: 17 and 18.

SEQ ID NO: 17 is the variable region of the heavy chain of antibody number 1-20, which specifically recognizes the RBD.

EVQLVESGGGLIQPGGSLRLSCAASGLTVSSNYMSWVRQAPGKGLEWVSV
IYSGGSTFYADSVKGRFTISRDNSKNTLYLQMNSLRAEDTAVYYCARDLF
YYGMDVWGQGTTVTVSS

SEQ ID NO: 18 is the variable region of the light chain of antibody number 1-20, which specifically recognizes the RBD.

DIQLTQSPSFLSASVGDRVTITCRASQGISSYLAWYQQKPGKAPKLLIYA
ASTLQSGVPSRFSGSGSGTEFTLTISSLQPEDFATYYCQQLNSYPCFGPG
TKVDIK

In one embodiment, the monoclonal antibody 2-30 comprises SEQ ID NOs: 19 and 20.

SEQ ID NO: 19 is the variable region of the heavy chain of antibody number 2-30, which specifically recognizes the RBD.

QVQLVESGGGVVQPGRSLRLSCAASGFTFSSYTMHWVRQAPGKGLEWVAA
ISYDGNNKYYADSVKGRFTISRDNSKNTLYLQMNSLRAEDTAVYYCARSI
LYGGGMDVWGQGTTVTVSS

SEQ ID NO: 20 is the variable region of the light chain of antibody number 2-30, which specifically recognizes the RBD.

DIQLTQSPSSLSASVGDRVTITCRASQGISSYLAWYQQKPGQAPKLLIYA
ASTLQSGVPSRFSGSGSGTDFTLTISSLQPEDFATYYCQQLNSYPLTFGG
GTKVEIK

In one embodiment, the monoclonal antibody 4-20 comprises SEQ ID NOs: 21 and 22.

SEQ ID NO: 21 is the variable region of the heavy chain of antibody number 4-20, which specifically recognizes the RBD.

QVQLLQSGAEVKKPGASVKVSCKASGYSFTSYYMHWVRQAPGQGLEWMGI
INPSGGGTTYAQKFQGRVTMTRDTSTSTVYMELSSLRSEDTAVYYCARPG
GGSYQEFDYWGQGTLVTVSS

SEQ ID NO: 22 is the variable region of the light chain of antibody number 4-20, which specifically recognizes the RBD.

DIQMTQSPSSLSASVGDRVTITCRASQSISSYLNWYQQKPGKAPKLLISA
ASSLQSGVPSRFSGSGSGTDFTLTISSLQPEDFATYYCQQSYNTLQVTFG
GGTKVEIK

In one embodiment, the monoclonal antibody 4-18 comprises SEQ ID NOs: 23 and 24.

SEQ ID NO: 23 is the variable region of the heavy chain of antibody number 4-18, which specifically recognizes the N-Terminal domain (NTD).

QVQLVESGGGVVQPGRSLRLSCAASGFTFSSYGMHWVRQAPGKGLEWVA
VISYDGSNKHYADSVKGRFTISRDNSKNTLYLQMNSLRAEDTAVYYCAK
DSGYNYGYSWFDPWGQGTLVTVSS

SEQ ID NO: 24 is the variable region of the light chain of antibody number 4-18, which specifically recognizes the NTD.

SYELTQPPSVSVSPGQTARITCSADALAKQYAYWYQQKPGQAPVLVIYK
DSERPSGIPERFSGSSSGTTVTLTISGVQAEDEADYYCQSTDNSGTYPN
WVFGGGTKLTVL

In one embodiment, the monoclonal antibody 5-24 comprises SEQ ID NOs: 25 and 26.

SEQ ID NO: 25 is the variable region of the heavy chain of antibody number 5-24, which specifically recognizes the NTD.

QVQLVESGGGVVQPGRSLRLSCAASGLTFSSYVMHWVRQAPGKGLDWVG
VIWYDGSKKYYADSVKGRFTISRDNSKNTLYLQMNSLRAEDTAVYYCAR
DPRDYYDFWSGYDYYYGLDVWGQGTTVTVSS

SEQ ID NO: 26 is the variable region of the light chain of antibody number 5-24, which specifically recognizes the NTD.

EIVLTQSPGILSLSPGERATLSCRASQSVSSSYLAWYQQKPGQAPRLLI
YGASSRATGIPDRFSGSGSGTDFTLTISRLEPEDFAVYYCQQYGSSGAL
TFGGGTKVEIK

In one embodiment, the monoclonal antibody 5-7 comprises SEQ ID NOs: 27 and 28.

SEQ ID NO: 27 is the variable region of the heavy chain of antibody number 5-7, which specifically recognizes the NTD.

QVQLVQSGAEVKKPGASVKVSCKASGYTFTSYYMHWVRQAPGQGLEWMG
VINPSGGSTSYAEKFRGRVTMTRDTSTSTVYMELSSLRSEDTAVYYCAR
DREPHSDSSGYWDSLKYYYYYALDVWGQGTTVTVSS

SEQ ID NO: 28 is the variable region of the light chain of antibody number 5-7, which specifically recognizes the NTD.

AIQLTQSPSSLSASVGDRVTITCRASQGISSYLAWYQQKPGKAPELLIY
AASTLQSGVPSRFSGSGSGTDFTLTISSLQPEDFATYYCQQLNTYPFTF
GPGTKVDIK

In one embodiment, the monoclonal antibody 1-68 comprises SEQ ID NOs: 29 and 30.

SEQ ID NO: 29 is the variable region of the heavy chain of antibody number 1-68, which specifically recognize the NTD.

QVQLVQSGAEVKKPGASVKVSCKVSGYTLIELSMHWVRQAPGKGLEWMGG
FDPEDAETTYAQKFQGRVTMTEDTSTDTAYMELSSLRSEDTAVYYCATGW
AVAGSSDVWYYYYGMDVWGQGTTVTVSS

SEQ ID NO: 30 is the variable region of the light chain of antibody number 1-68, which specifically recognizes the NTD.

QSALTQPPSVSGSPGQSVTISCTGTSSDVGSYNRVSWYQQPPGTAPKLMI
YEVSNRPSGVPDRFSGSKSGNTASLTISGLQAEDEADYYCSSYTSSSTYV
FGTGTKVTVL

In one embodiment, the monoclonal antibody 2-43 comprises SEQ ID NOs: 31 and 32.

SEQ ID NO: 31 is the variable region of the heavy chain of antibody number 2-43, which recognizes an epitope other than the RBD or the NTD.

QVQLVQSGAEVKKPGASVKVSCKASGYTFTGYYMHWVRQAPGQGLEWM
GWINPNSGGTNYAQKFQGRVTMTRDTSITTAYMELRRLRSDDTAVYYC
ARGLGVGCSGGNCYLDYYYMDVWGKGTTVTVSS

SEQ ID NO: 32 is the variable region of the light chain of antibody number 2-43, which recognizes an epitope other than the RBD or the NTD.

QSALTQPASVSGSPGQSITISCTGTSSDVGGYNYVSWYQQHPGKAPKLMI
YDVSKRPSGVSNRFSGSKSGNTASLTISGLQAEDEGDYYCSSYTSSSTWV
FGGGTKLTVL

In one embodiment, the monoclonal antibody 4-19 comprises SEQ ID NOs: 33 and 34.

SEQ ID NO: 33 is the variable region of the heavy chain of antibody number 4-19, which recognizes an epitope other than the RBD or the NTD.

QVQLQESGPGLVKPSETLSLTCTVSGGSISNYYWSWIRQSPGKGLEWIGY
IYYSGSTNYNPSLKSRVTISVDTSKNQFSLKLSSVTAADTAVYYCARSAK
HWLAPPGDYYYYMDVWGKGTTVTVSS

SEQ ID NO: 34 is the variable region of the light chain of antibody number 4-19, which recognizes an epitope other than the RBD or the NTD.

DIQLTQSPSFLSASVGDRVTITCRASQGISSYLAWYQQKPGKAPKLLIY
AASTLQSGVPSRFSGSGSGTEFTLTISSLQPEDFATYYCQQLNSYLTFG
GGTKVEIK

In one embodiment, the monoclonal antibody 2-17 comprises SEQ ID NOs: 35 and 36.

SEQ ID NO: 35 is the variable region of the heavy chain of antibody number 2-17, which recognizes an epitope other than the RBD or the NTD.

QVQLVQSGAEVKKPGSSVKVSCKASGGTFSSYAISWVRQAPGQGLEWMG
GNIPIFGTANYAQKFQGRVTITADESTSTAYMELSSLRSEDTAVYYCAR
GVGYRGVIPLNWFDPWGQGTVVTVSS

SEQ ID NO: 36 is the variable region of the light chain of antibody number 2-17, which recognizes an epitope other than the RBD or the NTD.

EIVMTQSPATLSVSPGERATLSCRASQSVSSDLAWYQHKPGQAPRLLIY
GASTRATGIPVRFSGSGSGTEFTLTISSLQSEDFAVYYCQQYNNWPPFT
FGGGTKVEIK

RBD binders

In one embodiment, the monoclonal antibody 2-36 comprises SEQ ID NOs: 37 and 38.

SEQ ID NO: 37 is the variable region of the heavy chain of antibody number 2-36, which specifically recognizes the RBD.

QVQLQESGPGLVKPSETLSLTCTVSGGSVSSSNYYWSWIRQPPGKGLEW
IGYMYYSGSTKYNPSLKSRVTISVDTSKNQFSLKLSSVTAADTAVYYCA
REVYYYDRSGYYASDGFDIWGQGTMVTVSS

SEQ ID NO: 38 is the variable region of the light chain of antibody number 2-36, which specifically recognizes the RBD.

EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPQTFG
QGTKVEIK

In one embodiment, the monoclonal antibody 1-87 comprises SEQ ID NOs: 39 and 40.

SEQ ID NO: 39 is the variable region of the heavy chain of antibody number 1-87, which specifically recognizes the NTD.

QVQLVQSGAEVKKPGASVKVSCKVSGYTLIELSMHWVRQAPGKGLEWMGG
FDPEDAETIYAQKFQGRVTMTEDTSTDTAYMELSSLRSEDTAVYYCATGI
AVIGPPPSTYYYYGMDVWGQGTTVTVSS

SEQ ID NO: 40 is the variable region of the light chain of antibody number 1-87, which specifically recognizes the NTD.

QSALTQPASVSGSPGQSITISCTGTSSDVGGYNYVSWYQQHPGKAPKLMI
YDVSKRPSGVSNRFSGSKSGNTASLTISGLQAEDEADYYCSSYTSSSTYV
FGTGTKVTVL

In one embodiment, the monoclonal antibody 2-15 mut comprises SEQ ID NOs: 41 and 42.

SEQ ID NO: 41 is the variable region of the heavy chain of the 2-15 mut mAb.

QVQLVQSGAEVKKPGASVRVSCKASGYTFTGYYMHWVRQAPGQGLEWMGW
INPISDGTNYAQKFQGWVTMTRDTSISTVYMELSRLRSDDTAVYYCARGG
SRCSGGNCYGWAYDAFDIWGQGTMITVSS

SEQ ID NO: 42 is the variable region of the light chain of the 2-15 mut mAb.

QSALTQPASVSGSPGQSITISCTGTSSDVGGYNFVSWYQQHPGKAPKLMI
YDVSKRPSGVSNRFSGSKSGNTASLTISGLQAEDEADSYCSSYTSSSTFV
FGTGTKVTVL

In one embodiment, the monoclonal antibody 4-8(39/51) comprises SEQ ID NOs: 43 and 44.

SEQ ID NO: 43 is the variable region of the heavy chain of the 4-8(39/51) mAb.

QVQLVQSGAEVKKAGSSVKVSCKASGGTFSSHTITWVRLAPGQGLEWMGR
NIPILGIANYAQKFQGRVTITADKSTSTAYMELSSLRSEDTAVYYCASLQ
TVDTAIEKYYGMDVWGQGTTVTVSS

SEQ ID NO: 44 is the variable region of the light chain of the 4-8(39/51) mAb.

SYELTQPPSVSVSPGQTASITCSGDKLGDKYACWYQQKPGQSPVLVIYQD
NKRPSGIPERFSGSNSGNTATLTISGTQAMDEADYYCQAWDSSTAVFGGG
TKLTVL

In one embodiment, the monoclonal antibody 4-8(39/51/57) comprises SEQ ID NOs: 45 and 46.

SEQ ID NO: 45 is the variable region of the heavy chain of the 4-8(39/51/57) mAb.

QVQLVQSGAEVKKAGSSVKVSCKASGGTFSSHTITWVRLAPGQGLEWMGR
NIPILGTANYAQKFQGRVTITADKSTSTAYMELSSLRSEDTAVYYCASLQ
TVDTAIEKYYGMDVWGQGTTVTVSS

SEQ ID NO: 46 is the variable region of the light chain of the 4-8(39/51/57) mAb.

SYELTQPPSVSVSPGQTASITCSGDKLGDKYACWYQQKPGQSPVLVIYQD
NKRPSGIPERFSGSNSGNTATLTISGTQAMDEADYYCQAWDSSTAVFGGG
TKLTVL

Nucleic acid sequences of the monoclonal antibodies described herein against SARS-CoV-2 or variant strains

In some embodiments, the subject matter disclosed herein relates to DNA sequences that encode any of the monoclonal antibodies disclosed herein. In some embodiments, the subject matter disclosed herein relates to mRNA sequences that encode any of the antibodies disclosed herein. As used herein, the terms “nucleic acid” and “nucleotide sequence” refer to both DNA and RNA molecules, including base-modified, sugar-modified or backbone-modified DNA or RNA molecules.

In some embodiments, the subject matter disclosed herein relates to an isolated nucleic acid having a nucleotide sequence encoding any of the antibodies of the present disclosure. In some embodiments, the nucleotide sequences encoding any of the antibodies of the present disclosure are codon-optimized for efficient expression in a host system. In some embodiments, the nucleotide sequences encoding any of the antibodies of the present disclosure are codon-optimized to increase the translational efficiency of the nucleotide sequences. In some embodiments, the nucleotide sequences encoding any of the antibodies of the present disclosure are codon-optimized to accommodate the codon bias of the host system. In some embodiments, the nucleotide sequences encoding any of the antibodies of the present disclosure are codon-optimized to remove redundancy and/or evolutionary constraints, including, but not limited to, the availability of tRNA isoacceptors, TATA box, Shine-Dalgarno sequences.

In some embodiments, the subject matter disclosed herein relates to a codon-optimized, isolated nucleic acid comprising a nucleic acid sequence encoding a monoclonal antibody including SEQ ID NO: 1. In some embodiments, the subject matter disclosed herein relates to a codon-optimized, isolated nucleic acid comprising a nucleic acid sequence encoding a monoclonal antibody including SEQ ID NO: 2. In some embodiments, the subject matter disclosed herein relates to a codon-optimized, isolated nucleic acid comprising a nucleic acid sequence encoding a monoclonal antibody including SEQ ID NO: 3. In some embodiments, the subject matter disclosed herein relates to a codon-optimized, isolated nucleic acid comprising a nucleic acid sequence encoding a monoclonal antibody including SEQ ID NO: 4. In some embodiments, the subject matter disclosed herein relates to a codon-optimized, isolated nucleic acid comprising a nucleic acid sequence encoding a monoclonal antibody including SEQ ID NO: 5. In some embodiments, the subject matter disclosed herein relates to a codon-optimized, isolated nucleic acid comprising a nucleic acid sequence encoding a monoclonal antibody including SEQ ID NO: 6. In some embodiments, the subject matter disclosed herein relates to a codon-optimized, isolated nucleic acid comprising a nucleic acid sequence encoding a monoclonal antibody including SEQ ID NO: 7. In some embodiments, the subject matter disclosed herein relates to a codon-optimized, isolated nucleic acid comprising a nucleic acid sequence encoding a monoclonal antibody including SEQ ID NO: 8. In some embodiments, the subject matter disclosed herein relates to a codon-optimized, isolated nucleic acid comprising a nucleic acid sequence encoding a monoclonal antibody including SEQ ID NO: 9. In some embodiments, the subject matter disclosed herein relates to a codon-optimized, isolated nucleic acid comprising a nucleic acid sequence encoding a monoclonal antibody including SEQ ID NO: 10. In some embodiments, the subject matter disclosed herein relates to a codon-optimized, isolated nucleic acid comprising a nucleic acid sequence encoding a monoclonal antibody including SEQ ID NO: 11. In some embodiments, the subject matter disclosed herein relates to a codon-optimized, isolated nucleic acid comprising a nucleic acid sequence encoding a monoclonal antibody including SEQ ID NO: 12. In some embodiments, the subject matter disclosed herein relates to a codon-optimized, isolated nucleic acid comprising a nucleic acid sequence encoding a monoclonal antibody including SEQ ID NO: 13. In some embodiments, the subject matter disclosed herein relates to a codon-optimized, isolated nucleic acid comprising a nucleic acid sequence encoding a monoclonal antibody including SEQ ID NO: 14. In some embodiments, the subject matter disclosed herein relates to a codon-optimized, isolated nucleic acid comprising a nucleic acid sequence encoding a monoclonal antibody including SEQ ID NO: 15. In some embodiments, the subject matter disclosed herein relates to a codon-optimized, isolated nucleic acid comprising a nucleic acid sequence encoding a monoclonal antibody including SEQ ID NO: 16. In some embodiments, the subject matter disclosed herein relates to a codon-optimized, isolated nucleic acid comprising a nucleic acid sequence encoding a monoclonal antibody including SEQ ID NO: 17. In some embodiments, the subject matter disclosed herein relates to a codon-optimized, isolated nucleic acid comprising a nucleic acid sequence encoding a monoclonal antibody including SEQ ID NO: 18. In some embodiments, the subject matter disclosed herein relates to a codon-optimized, isolated nucleic acid comprising a nucleic acid sequence encoding a monoclonal antibody including SEQ ID NO: 19. In some embodiments, the subject matter disclosed herein relates to a codon-optimized, isolated nucleic acid comprising a nucleic acid sequence encoding a monoclonal antibody including SEQ ID NO: 20. In some embodiments, the subject matter disclosed herein relates to a codon-optimized, isolated nucleic acid comprising a nucleic acid sequence encoding a monoclonal antibody including SEQ ID NO: 21. In some embodiments, the subject matter disclosed herein relates to a codon-optimized, isolated nucleic acid comprising a nucleic acid sequence encoding a monoclonal antibody including SEQ ID NO: 22. In some embodiments, the subject matter disclosed herein relates to a codon-optimized, isolated nucleic acid comprising a nucleic acid sequence encoding a monoclonal antibody including SEQ ID NO: 23. In some embodiments, the subject matter disclosed herein relates to a codon-optimized, isolated nucleic acid comprising a nucleic acid sequence encoding a monoclonal antibody including SEQ ID NO: 24. In some embodiments, the subject matter disclosed herein relates to a codon-optimized, isolated nucleic acid comprising a nucleic acid sequence encoding a monoclonal antibody including SEQ ID NO: 25. In some embodiments, the subject matter disclosed herein relates to a codon-optimized, isolated nucleic acid comprising a nucleic acid sequence encoding a monoclonal antibody including SEQ ID NO: 26. In some embodiments, the subject matter disclosed herein relates to a codon-optimized, isolated nucleic acid comprising a nucleic acid sequence encoding a monoclonal antibody including SEQ ID NO: 27. In some embodiments, the subject matter disclosed herein relates to a codon-optimized, isolated nucleic acid comprising a nucleic acid sequence encoding a monoclonal antibody including SEQ ID NO: 28. In some embodiments, the subject matter disclosed herein relates to a codon-optimized, isolated nucleic acid comprising a nucleic acid sequence encoding a monoclonal antibody including SEQ ID NO: 29. In some embodiments, the subject matter disclosed herein relates to a codon-optimized, isolated nucleic acid comprising a nucleic acid sequence encoding a monoclonal antibody including SEQ ID NO: 30. In some embodiments, the subject matter disclosed herein relates to a codon-optimized, isolated nucleic acid comprising a nucleic acid sequence encoding a monoclonal antibody including SEQ ID NO: 31. In some embodiments, the subject matter disclosed herein relates to a codon-optimized, isolated nucleic acid comprising a nucleic acid sequence encoding a monoclonal antibody including SEQ ID NO: 32. In some embodiments, the subject matter disclosed herein relates to a codon-optimized, isolated nucleic acid comprising a nucleic acid sequence encoding a monoclonal antibody including SEQ ID NO: 33. In some embodiments, the subject matter disclosed herein relates to a codon-optimized, isolated nucleic acid comprising a nucleic acid sequence encoding a monoclonal antibody including SEQ ID NO: 34. In some embodiments, the subject matter disclosed herein relates to a codon-optimized, isolated nucleic acid comprising a nucleic acid sequence encoding a monoclonal antibody including SEQ ID NO: 35. In some embodiments, the subject matter disclosed herein relates to a codon-optimized, isolated nucleic acid comprising a nucleic acid sequence encoding a monoclonal antibody including SEQ ID NO: 36. In some embodiments, the subject matter disclosed herein relates to a codon-optimized, isolated nucleic acid comprising a nucleic acid sequence encoding a monoclonal antibody including SEQ ID NO: 37. In some embodiments, the subject matter disclosed herein relates to a codon-optimized, isolated nucleic acid comprising a nucleic acid sequence encoding a monoclonal antibody including SEQ ID NO: 38. In some embodiments, the subject matter disclosed herein relates to a codon-optimized, isolated nucleic acid comprising a nucleic acid sequence encoding a monoclonal antibody including SEQ ID NO: 39. In some embodiments, the subject matter disclosed herein relates to a codon-optimized, isolated nucleic acid comprising a nucleic acid sequence encoding a monoclonal antibody including SEQ ID NO: 40. In some embodiments, the subject matter disclosed herein relates to a codon-optimized, isolated nucleic acid comprising a nucleic acid sequence encoding a monoclonal antibody including SEQ ID NO: 41. In some embodiments, the subject matter disclosed herein relates to a codon-optimized, isolated nucleic acid comprising a nucleic acid sequence encoding a monoclonal antibody including SEQ ID NO: 42. In some embodiments, the subject matter disclosed herein relates to a codon-optimized, isolated nucleic acid comprising a nucleic acid sequence encoding a monoclonal antibody including SEQ ID NO: 43. In some embodiments, the subject matter disclosed herein relates to a codon-optimized, isolated nucleic acid comprising a nucleic acid sequence encoding a monoclonal antibody including SEQ ID NO: 44. In some embodiments, the subject matter disclosed herein relates to a codon-optimized, isolated nucleic acid comprising a nucleic acid sequence encoding a monoclonal antibody including SEQ ID NO: 45. In some embodiments, the subject matter disclosed herein relates to a codon-optimized, isolated nucleic acid comprising a nucleic acid sequence encoding a monoclonal antibody including SEQ ID NO: 46.

In some embodiments, the subject matter disclosed herein relates to a pharmaceutical composition comprising an isolated nucleic acid having a nucleotide sequence encoding any of the antibodies of the present disclosure and a pharmaceutically acceptable carrier. In some embodiments, the subject matter disclosed herein relates to a pharmaceutical composition comprising a codon-optimized, isolated nucleic acid having a nucleotide sequence encoding any of the antibodies of the present disclosure and a pharmaceutically acceptable carrier.

COVID-19 Therapeutics with Bispecific Antibodies

Monoclonal antibodies are homogeneous in their nature of interaction with the ligands/antigens and thereby generate an absolute mono-specificity for their target. However, sometimes this absolute specificity for a single antigen target can result in some limitations for treating agents such as viruses that are known to evolve their target epitopes and thereby escape from the targeting by monoclonal antibodies. To overcome such limitations, scientists have generated antibodies which can be engineered to target multiple epitopes using a single antibody-like molecule. These antibody-like molecules can bind two separate antigens at the same time and are referred to as “bispecific” antibodies. Their framework is composed of fragments of two monoclonal antibodies whose regions have a binding affinity to two separate antigens. Such a framework can be useful in binding antigens that are unconventional or rare. In some embodiments, the subject matter described herein relates to generating bispecific antibodies capable of neutralizing the SARS-CoV-2 virus and or variant strains.

The SARS-CoV-2 bispecific antibodies described herein can be used for treatment of subjects infected with SARS-CoV-2 or a variant strain. In some embodiments, the bispecific antibodies described herein reduce viral load in a subject in need thereof. In some embodiments, the bispecific antibodies described herein decrease disease severity in a subject in need thereof. In some embodiments, the bispecific antibodies described herein improve clinical outcome of COVID-19 in subjects in need thereof.

In some embodiments, the bispecific antibodies described herein can be used as prophylaxis to prevent high risk subjects and individuals from becoming infected with SARS-CoV-2 or a variant strain. In some embodiments, the high risk subject can be healthcare workers in close proximity to COVID-19 patients or the family members of these healthcare workers. In some embodiments, the bispecific antibodies described herein can be used as prophylaxis for the general population at large.

In some embodiments, more potent antibodies require smaller amounts to be administered to the subject to achieve the desired COVID-19-related recovery effects. This can be through lower dosages administered to the subjects and therefore less potential side effects, less frequent dosing regimens, or lower antibody production costs. In some embodiments, potency is measured by IC50 and IC90 concentration values.

In some embodiments, the bispecific antibodies described herein target multiple epitopes on the SARS-CoV-2 virus or a variant strain. In some embodiments, the subject matter disclosed herein relates to bispecific antibody combination cocktails. In some embodiments, the antibodies in the cocktails target multiple epitopes on the SARS-CoV-2 virus or a variant strain. In some embodiments, the bispecific antibody combination cocktails provide even greater efficacy and lower the possibility of viral resistance than each individual antibody of the cocktail. In some embodiments, the bispecific antibodies described herein can be utilized in laboratory research and development activities. In one embodiment, the bispecific antibodies describe herein includes the variable regions of a first and a second antibody, in which the first antibody binds to an epitope on the receptor binding domain (RBD) of the coronavirus, while the second binds to an epitope on the N-terminal domain (NTD) of the coronavirus.

Pairing the anti-RBD and the anti-NTD domain antibodies in such a bispecific format would mainly provide for increasing the genetic barrier for the virus to achieve resistance against the antibodies. It also allows clinical practitioners to only administer a single formulation of one bispecific antibody to patients thereby simplifying treatment. In some embodiments, the bispecific antibodies are administered in a cocktail of two or more antibodies. As for marketing and supply chain benefits, bispecific antibodies present an economical alternative to producing in a single molecule format, those combinations of monoclonal antibodies known to increase the threshold of resistance to SARS-CoV-2.

In one embodiment, the subject matter disclosed herein relates to bispecific antibodies engineered for treatment and prevention of COVID-19 infections. Engineered bispecific antibodies can combine the binding specificity of two antibodies in only one molecule. Bispecific antibodies can recognize two antigens on different cells or on the same cell. Thus, by virtue of specifically binding the two antigens, engineered bispecific antibodies can bring two cells in close proximity or activate two receptors simultaneously. Additionally, blocking two target proteins with one antibody instead of two may be more efficient. In some embodiments, bispecific antibodies can target two viral epitopes providing higher barrier for viral escape. In some embodiments, bispecific antibodies can have synergetic effect on potency against the viral target.

In one embodiment, the engineered antibodies achieve bispecificity using the CrossMab format (Schaefer, 2011). The CrossMab engineering format allows for the bispecific antibody to adopt a more native antibody-like structure. This can facilitate binding of the bispecific antibody to weakly-expressed antigens, while avoiding overstimulation of immune responses. In some embodiments, bispecific antibodies in the CrossMab format can be anchored to a host cell membrane. This allows for improved local antibody concentration, targeting of sequential and/or interdependent virus entry steps, and compensating for monovalent binding.

As described herein, the CrossMab format for engineering bispecific antibodies can be utilized to engineer bispecific antibodies against the SARS-CoV-2 virus or a variant strain. As shown in FIG. 41, the creation of a “knob” in one heavy chain and a “hole” in the other heavy chain of the bispecific antibody favors the formation of heavy chain heterodimers, while the “crossover” of CL and CH1 sequences (the constant domains, heavy and light chains) in one arm of the antibody favors correct Heavy-Light chain pairings in both arms. The CrossMab format allows for correct assembly of two heavy chains and two light chains from different parental antibodies into one bispecific antibody molecule that resembles a typical monoclonal antibody in terms of mass and architecture, and with no artificial linkers required. Each antibody arm can be selected from different potent neutralizing antibodies against SARS-CoV-2 or a variant strain. Other bispecific formats that bring together the particular SARS-CoV-2 antibody combinations described herein can yield similar neutralization activities. As shown in FIG. 41, human IgG isotype 1 Fc can be engineered for reduced Fc-effector function with the L234F, L235E and P331S mutations and for half-life with the M428L and N434S mutations.

In one embodiment, the bispecific antibody comprises two polypeptide chains, one from each parental monoclonal antibody. In one embodiment, the first parental polypeptide arm comprises the light chain and the heavy chain polypeptides of the first parental antibody. The light chain can include the light chain variable domain (VL) comprising the light chain binding region and the light chain constant domain (CL) if the first parental antibody. The heavy chain can include the heavy chain variable domain (VH) comprising the heavy chain binding region and the heavy chain constant domain (CH) of the first parental antibody. In one embodiment, the first parental polypeptide arm also includes a domain, which promotes heterodimerization with the second polypeptide. In one embodiment, this heterodimerization domain can covalently bond the first parental polypeptide to the second parental polypeptide of the bispecific antibody.

In one embodiment, the second parental polypeptide arm comprises a light chain and a heavy chain polypeptides of the second parental antibody. The light chain can include the light chain variable domain (VL) comprising the light chain binding region and the light chain constant domain (CL) of the second parental antibody. The heavy chain can include the heavy chain variable domain (VH) comprising the heavy chain binding region and the heavy chain constant domain (CH) of the second parental antibody. In one embodiment, the second parental polypeptide arm also includes a domain, which promotes heterodimerization with the second polypeptide. In one embodiment, this heterodimerization domain can covalently bond the first parental polypeptide to the second parental polypeptide of the bispecific antibody. In one embodiment, the bispecific antibody further comprises linkers and disulfide bonds connecting the two parental polypeptides of the antibody. The linkers can be any suitable sequence of amino acids. In one embodiment, the The bispecific antibodies described herein include the variable regions of a first and a second antibody, in which the first antibody binds to an epitope on the receptor binding domain (RBD) of the coronavirus and is selected from 8 monoclonal Abs (1-20, 1-57, 2-7, 2-15, 2-30, 2-36, 2-43 and 4-20) while the second binds to an epitope on the N-terminal domain (NTD) of the coronavirus and is also selected from 8 monoclonal Abs (1-68, 1-87, 2-17, 2-51, 4-8, 4-18, 4-19 and 5-24).

The CrossMabs described herein utilize human IgG isotype 1 Fc backbone. The backbone can have additional mutations engineered for reduced Fc-effector function with the L234F, L235E and P331S mutations and for half-life with the M428L and N434S mutations.

In order to generate bispecific antibodies that would meet these goals, 58 candidate bispecific Abs were screened for their potency against SARS-CoV-2 live infectious virus using an in vitro assay. Briefly, purified candidate bispecific Ab was mixed with infectious live virus for 60 min and then added to a monolayer of Vero E6 cells to measure the extent of neutralization. Antibodies were diluted 5 fold from 10 μg/mL while virus was incubated with cells at MOI=0.1. Three days post infection, the cells were measured for the cytopathic effects induced by the live virus replication. Difference in the extent of infection from the control wells bearing no antibodies was converted to percentage and the value for inhibition of 50% of the virus (IC50) and 90% of the virus (IC90) was calculated using non-linear regression analysis.

Using the potency values, the bispecific antibodies of merit were nominated based on their fold enhancement of the IC₉₀ concentrations that they achieved in comparison to their parental antibodies. Bispecific antibodies that had a potency with IC₅₀≤10 ng/mL and with IC₉₀≤50 ng/mL were chosen as the ones that would be capable of achieving the greatest barrier to resistance by SARS-CoV-2. Ab X and Ab Y are irrelevant single arm Fab controls. Selected bispecific antibody candidates achieve similar potency as co-administration of the two parental mAbs.

SARS-CoV-2 Variant Strains

In some embodiments, the bispecific antibodies exhibit neutralization activity against wild-type SARS-CoV-2 virus (WA1). In some embodiments, the bispecific antibodies exhibit neutralization activity against SARS-CoV-2 variants. In some embodiments, the bispecific antibodies disclosed herein show potent neutralization activities against SARS-CoV-2 wild-type and multiple variant virus strains. In some embodiments, the SARS-CoV-2 variants are B.1.1.7 (UK variant), B.1.351 (SA variant), B.1.526 (NY variant), or P.1 (Brazil variant).

In one embodiment, each arm of the bispecific antibody constructed is selected from 19 potent neutralizing antibodies against SARS-CoV-2. Additional bispecific antibodies 2-7/5-7, 2-7/1-57 and the CrossMab formats with reverse the knob and hole arms, namely 5-7/2-7 or 1-57/2-7, showed high potencies when tested against the wild-type virus. As 1-57 activity can be impacted by SARS-CoV-2 variant containing L452R mutation (such as California variant), which crashes mAb 1-57 heavy chain binding suggested by structural modeling, 2-7/5-7 was down-selected as development candidate and further characterized by neutralization against SARS-CoV-2 variants and showed potent neutralization against new emerging variants such as B.1.1.7 (UK variant), B.1.351 (SA variant), B.1.526 (NY variant) and P.1 (BZ variant). For many of the bispecific antibodies tested herein, both arms of the bispecific antibodies contribute to the neutralization activity of the bispecific molecule. These bispecific antibodies with potent neutralization activities against SARS-CoV-2 and the variants offer new therapeutics for the treatment and prevention of COVID-19, as a single antibody molecule to provide a higher genetic barrier for viral escape and lower cost than antibody combination. We expect the activity enhancement observed can be extended to other engineered bispecific antibody format.

Antibody Sequences for Bispecific Antibodies

Underlined and italicized amino acids represent the respective complementarity-determining regions (CDRs). There are three CDRs per sequence, appearing on the sequence in the order CDR1, CDR2, and CDR3.

The 2-17/2-7 bispecific antibody sequence can comprise:

SEQ ID NO: 47 is the amino acid sequence defining the 2-17 derived heavy chain of the 2-17/2-7 antibody 2-17HC-Hole-Cross

QVQLVQSGAEVKKPGSSVKVSCKASGGTFSSYAISWVRQAPGQGLEWMGG
NIPIFGTANYAQKFQGRVTITADESTSTAYMELSSLRSEDTAVYYCARGV
GYRGVIPLNWFDPWGQGTVVTVSSASTAAPSVFIFPPSDEQLKSGTASVV
CLLNNFYPREAKVQWKVDNALQSGNSQESVTEQDSKDSTYSLSSTLTLSK
ADYEKHKVYACEVTHQGLSSPVTKSFNRGECDKTHTCPPCPAPEFEGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPASIEKTISKA
KGQPREPQVCTLPPSRDELTKNQVSLSCAVKGFYPSDIAVEWESNGQPEN
NYKTTPPVLDSDGSFFLVSKLTVDKSRWQQGNVFSCSVLHEALHSHYTQK
SLSLSPGK

SEQ ID NO: 48 is the amino acid sequence defining the 2-17 derived Light chain of the 2-17/2-7 antibody 2-17VLCH1

EIVMTQSPATLSVSPGERATLSCRASQSVSSDLAWYQHKPGQAPRLLIYG
ASTRATGIPVRFSGSGSGTEFTLTISSLQSEDFAVYYCQQYNNWPPFTFG
GGTKVEIKSSASTKGPSVFPLAPSSKSTSGGTAALGCLVKDYFPEPVTVS
WNSGALTSGVHTFPAVLQSSGLYSLSSVVTVPSSSLGTQTYICNVNHKPS
NTKVDKKVEPKSC

SEQ ID NO: 49 is the amino acid sequence defining the 2-7 derived heavy chain of the 2-17/2-7 antibody 2-7HC-Knob

QITLKESGPTLVKPTQTLTLTCTFSGFSLSTSGVGVGWIRQPPGKALEWL
ALIYWDDDKRYSPSLKSRLTITKDTSKNQVVLTMTNMDPVDTATYYCAHH
KIERIFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPEFEGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPASIEKTISKAKGQPREPQV
YTLPPCRDELTKNQVSLWCLVKGFYPSDIAVEWESNGQPENNYKTTPPVL
DSDGSFFLYSKLTVDKSRWQQGNVFSCSVLHEALHSHYTQKSLSLSPGK

SEQ ID NO: 50 is the amino acid sequence defining the 2-7 derived light chain of the 2-17/2-7 antibody 2-7LC

QSALAQPASVSGSPGQSITISCTGTSSDVGAYNYVSWYQQHPGKAPKLMI
YDVSKRPSGVSNRFSGSKSGNTASLTISGLQAEDEADYYCSSYTTSSTVF
GGGTKLTVLGQPKAAPSVTLFPPSSEELQANKATLVCLISDFYPGAVTVA
WKADSSPVKAGVETTTPSKQSNNKYAASSYLSLTPEQWKSHRSYSCQVTH
EGSTVEKTVAPTECS

The 1-57/5-7 bispecific antibody sequence can comprise:

SEQ ID NO: 51 is the amino acid sequence defining the 1-57 derived heavy chain of the 1-57/5-7 antibody 1-57HC-Hole-Cross

EVHLVESGGGLVQPGGSLRLSCAASGFTFSDHYMDWVRQAPGKGLEWVGR
TRNKANSYTTEYAASVKGRFTISRDDAKNSLYLQMNSLKTEDTAVYYCAR
VHRWAYCINGVCFGAYSDYWGQGTLVTVSSASTAAPSVFIFPPSDEQLKS
GTASVVCLLNNFYPREAKVQWKVDNALQSGNSQESVTEQDSKDSTYSLSS
TLTLSKADYEKHKVYACEVTHQGLSSPVTKSFNRGECDKTHTCPPCPAPE
FEGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPASIE
KTISKAKGQPREPQVCTLPPSRDELTKNQVSLSCAVKGFYPSDIAVEWES
NGQPENNYKTTPPVLDSDGSFFLVSKLTVDKSRWQQGNVFSCSVLHEALH
SHYTQKSLSLSPGK

SEQ ID NO: 52 is the amino acid sequence defining the 1-57 derived light chain of the 1-57/5-7 antibody 1-57VLCH1

EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPSTFG
QGTKLEIKSSASTKGPSVFPLAPSSKSTSGGTAALGCLVKDYFPEPVTVS
WNSGALTSGVHTFPAVLQSSGLYSLSSVVTVPSSSLGTQTYICNVNHKPS
NTKVDKKVEPKSC

SEQ ID NO: 53 is the amino acid sequence defining the 5-7 derived heavy chain of the 1-57/5-7 antibody 5-7 HC-Knob

QVQLVQSGAEVKKPGASVKVSCKASGYTFTSYYMHWVRQAPGQGLEWMGV
INPSGGSTSYAEKFRGRVTMTRDTSTSTVYMELSSLRSEDTAVYYCARDR
EPHSDSSGYWDSLKYYYYYALDVWGQGTTVTVSSASTKGPSVFPLAPSSK
STSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPE
FEGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPASIE
KTISKAKGQPREPQVYTLPPCRDELTKNQVSLWCLVKGFYPSDIAVEWES
NGQPENNYKTTPPVLDSDGSFFLYSKLTVDKSRWQQGNVFSCSVLHEALH
SHYTQKSLSLSPGK

SEQ ID NO: 54 is the amino acid sequence defining the 5-7 derived light chain of the 1-57/5-7 antibody 5-7LC

AIQLTQSPSSLSASVGDRVTITCRASQGISSYLAWYQQKPGKAPELLIYA
ASTLQSGVPSRFSGSGSGTDFTLTISSLQPEDFATYYCQQLNTYPFTFGP
GTKVDIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

The 5-7/1-57 bispecific antibody sequence can comprise:

SEQ ID NO: 55 is the amino acid sequence defining the 5-7 derived heavy chain of the 5-7/1-57 antibody 5-7HC-Hole-Cross

QVQLVQSGAEVKKPGASVKVSCKASGYTFTSYYMHWVRQAPGQGLEWMGV
INPSGGSTSYAEKFRGRVTMTRDTSTSTVYMELSSLRSEDTAVYYCARDR
EPHSDSSGYWDSLKYYYYYALDVWGQGTTVTVSSASTAAPSVFIFPPSDE
QLKSGTASVVCLLNNFYPREAKVQWKVDNALQSGNSQESVTEQDSKDSTY
SLSSTLTLSKADYEKHKVYACEVTHQGLSSPVTKSFNRGECDKTHTCPPC
PAPEFEGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
ASIEKTISKAKGQPREPQVCTLPPSRDELTKNQVSLSCAVKGFYPSDIAV
EWESNGQPENNYKTTPPVLDSDGSFFLVSKLTVDKSRWQQGNVFSCSVLH
EALHSHYTQKSLSLSPGK

SEQ ID NO: 56 is the amino acid sequence defining the 5-7 derived Light chain of the 5-7/1-57 antibody 5-7VLCH1

AIQLTQSPSSLSASVGDRVTITCRASQGISSYLAWYQQKPGKAPELLIYA
ASTLQSGVPSRFSGSGSGTDFTLTISSLQPEDFATYYCQQLNTYPFTFGP
GTKVDIKSSASTKGPSVFPLAPSSKSTSGGTAALGCLVKDYFPEPVTVSW
NSGALTSGVHTFPAVLQSSGLYSLSSVVTVPSSSLGTQTYICNVNHKPSN
TKVDKKVEPKSC

SEQ ID NO: 57 is the amino acid sequence defining the 1-57 derived heavy chain of the 5-7/1-57 antibody 1-57HC-Knob

EVHLVESGGGLVQPGGSLRLSCAASGFTFSDHYMDWVRQAPGKGLEWVGR
TRNKANSYTTEYAASVKGRFTISRDDAKNSLYLQMNSLKTEDTAVYYCAR
VHRWAYCINGVCFGAYSDYWGQGTLVTVSSASTKGPSVFPLAPSSKSTSG
GTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEFEGG
PSVFLFPPKPKDTLMISRTPEVTCVVVDVSHEDPEVKFNWYVDGVEVHNA
KTKPREEQYNSTYRVVSVLTVLHQDWLNGKEYKCKVSNKALPASIEKTIS
KAKGQPREPQVYTLPPCRDELTKNQVSLWCLVKGFYPSDIAVEWESNGQP
ENNYKTTPPVLDSDGSFFLYSKLTVDKSRWQQGNVFSCSVLHEALHSHYT
QKSLSLSPGK

SEQ ID NO: 58 is the amino acid sequence defining the 1-57 derived light chain of the 5-7/1-57 antibody 1-57LC

EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPSTFG
QGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC

The 2-7/5-7 bispecific antibody sequence can comprise:

SEQ ID NO: 59 is the amino acid sequence defining the 2-7 derived heavy chain of the 2-7/5-7 antibody 2-7HC-Hole-Cross

QITLKESGPTLVKPTQTLTLTCTFSGFSLSTSGVGVGWIRQPPGKALEWL
ALIYWDDDKRYSPSLKSRLTITKDTSKNQVVLTMTNMDPVDTATYYCAHH
KIERIFDYWGQGTLVTVSSASTAAPSVFIFPPSDEQLKSGTASVVCLLNN
FYPREAKVQWKVDNALQSGNSQESVTEQDSKDSTYSLSSTLTLSKADYEK
HKVYACEVTHQGLSSPVTKSFNRGECDKTHTCPPCPAPEFEGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPASIEKTISKAKGQPR
EPQVCTLPPSRDELTKNQVSLSCAVKGFYPSDIAVEWESNGQPENNYKTT
PPVLDSDGSFFLVSKLTVDKSRWQQGNVFSCSVLHEALHSHYTQKSLSLS
PGK

SEQ ID NO: 60 is the amino acid sequence defining the 2-7 derived light chain of the 2-7/5-7 antibody 2-7VLCH1

QSALAQPASVSGSPGQSITISCTGTSSDVGAYNYVSWYQQHPGKAPKLMI
YDVSKRPSGVSNRFSGSKSGNTASLTISGLQAEDEADYYCSSYTTSSTVF
GGGTKLTVLSSASTKGPSVFPLAPSSKSTSGGTAALGCLVKDYFPEPVTV
SWNSGALTSGVHTFPAVLQSSGLYSLSSVVTVPSSSLGTQTYICNVNHKP
SNTKVDKKVEPKSC

SEQ ID NO: 61 is the amino acid sequence defining the 5-7 derived heavy chain of the 2-7/5-7 antibody 5-7 HC-Knob

QVQLVQSGAEVKKPGASVKVSCKASGYTFTSYYMHWVRQAPGQGLEWMGV
INPSGGSTSYAEKFRGRVTMTRDTSTSTVYMELSSLRSEDTAVYYCARDR
EPHSDSSGYWDSLKYYYYYALDVWGQGTTVTVSSASTKGPSVFPLAPSSK
STSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPE
FEGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPASIE
KTISKAKGQPREPQVYTLPPCRDELTKNQVSLWCLVKGFYPSDIAVEWES
NGQPENNYKTTPPVLDSDGSFFLYSKLTVDKSRWQQGNVFSCSVLHEALH
SHYTQKSLSLSPGK

SEQ ID NO: 62 is the amino acid sequence defining the 5-7 derived light chain of the 2-7/5-7 antibody 5-7LC

AIQLTQSPSSLSASVGDRVTITCRASQGISSYLAWYQQKPGKAPELLIYA
ASTLQSGVPSRFSGSGSGTDFTLTISSLQPEDFATYYCQQLNTYPFTFGP
GTKVDIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

The 5-7/2-7 bispecific antibody sequence can comprise:

SEQ ID NO: 63 is the amino acid sequence defining the 5-7 derived heavy chain of the 5-7/2-7 antibody 5-7HC-Hole-Cross

QVQLVQSGAEVKKPGASVKVSCKASGYTFTSYYMHWVRQAPGQGLEWMGV
INPSGGSTSYAEKFRGRVTMTRDTSTSTVYMELSSLRSEDTAVYYCARDR
EPHSDSSGYWDSLKYYYYYALDVWGQGTTVTVSSASTAAPSVFIFPPSDE
QLKSGTASVVCLLNNFYPREAKVQWKVDNALQSGNSQESVTEQDSKDSTY
SLSSTLTLSKADYEKHKVYACEVTHQGLSSPVTKSFNRGECDKTHTCPPC
PAPEFEGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
ASIEKTISKAKGQPREPQVCTLPPSRDELTKNQVSLSCAVKGFYPSDIAV
EWESNGQPENNYKTTPPVLDSDGSFFLVSKLTVDKSRWQQGNVFSCSVLH
EALHSHYTQKSLSLSPGK

SEQ ID NO: 64 is the amino acid sequence defining the 5-7 derived Light chain of the 5-7/2-7 antibody 5-7VLCH1

AIQLTQSPSSLSASVGDRVTITCRASQGISSYLAWYQQKPGKAPELLIYA
ASTLQSGVPSRFSGSGSGTDFTLTISSLQPEDFATYYCQQLNTYPFTFGP
GTKVDIKSSASTKGPSVFPLAPSSKSTSGGTAALGCLVKDYFPEPVTVSW
NSGALTSGVHTFPAVLQSSGLYSLSSVVTVPSSSLGTQTYICNVNHKPSN
TKVDKKVEPKSC

SEQ ID NO: 65 is the amino acid sequence defining the 2-7 derived heavy chain of the 5-7/2-7 antibody 2-7HC-Knob

QITLKESGPTLVKPTQTLTLTCTFSGFSLSTSGVGVGWIRQPPGKALEWL
ALIYWDDDKRYSPSLKSRLTITKDTSKNQVVLTMTNMDPVDTATYYCAHH
KIERIFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPEFEGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPASIEKTISKAKGQPREPQV
YTLPPCRDELTKNQVSLWCLVKGFYPSDIAVEWESNGQPENNYKTTPPVL
DSDGSFFLYSKLTVDKSRWQQGNVFSCSVLHEALHSHYTQKSLSLSPGK

SEQ ID NO: 66 is the amino acid sequence defining the 2-7 derived light chain of the 5-7/2-7 antibody 2-7LC

QSALAQPASVSGSPGQSITISCTGTSSDVGAYNYVSWYQQHPGKAPKLMI
YDVSKRPSGVSNRFSGSKSGNTASLTISGLQAEDEADYYCSSYTTSSTVF
GGGTKLTVLGQPKAAPSVTLFPPSSEELQANKATLVCLISDFYPGAVTVA
WKADSSPVKAGVETTTPSKQSNNKYAASSYLSLTPEQWKSHRSYSCQVTH
EGSTVEKTVAPTECS

The 2-17/1-57 bispecific antibody sequence can comprise:

SEQ ID NO: 67 is the amino acid sequence defining the 2-17 derived heavy chain of the 2-17/1-57 antibody 2-17HC-Hole-Cross

QVQLVQSGAEVKKPGSSVKVSCKASGGTFSSYAISWVRQAPGQGLEWMGG
NIPIFGTANYAQKFQGRVTITADESTSTAYMELSSLRSEDTAVYYCARGV
GYRGVIPLNWFDPWGQGTVVTVSSASTAAPSVFIFPPSDEQLKSGTASVV
CLLNNFYPREAKVQWKVDNALQSGNSQESVTEQDSKDSTYSLSSTLTLSK
ADYEKHKVYACEVTHQGLSSPVTKSFNRGECDKTHTCPPCPAPEFEGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPASIEKTISKA
KGQPREPQVCTLPPSRDELTKNQVSLSCAVKGFYPSDIAVEWESNGQPEN
NYKTTPPVLDSDGSFFLVSKLTVDKSRWQQGNVFSCSVLHEALHSHYTQK
SLSLSPGK

SEQ ID NO: 68 is the amino acid sequence defining the 2-17 derived light chain of the 2-17/1-57 antibody 2-17VLCH1

EIVMTQSPATLSVSPGERATLSCRASQSVSSDLAWYQHKPGQAPRLLIY
GASTRATGIPVRFSGSGSGTEFTLTISSLQSEDFAVYYCQQYNNWPPFT
FGGGTKVEIKSSASTKGPSVFPLAPSSKSTSGGTAALGCLVKDYFPEPV
TVSWNSGALTSGVHTFPAVLQSSGLYSLSSVVTVPSSSLGTQTYICNVN
HKPSNTKVDKKVEPKSC

SEQ ID NO: 69 is the amino acid sequence defining the 1-57 derived heavy chain of the 2-17/1-57 antibody 1-57HC-Knob

EVHLVESGGGLVQPGGSLRLSCAASGFTFSDHYMDWVRQAPGKGLEWVG
RTRNKANSYTTEYAASVKGRFTISRDDAKNSLYLQMNSLKTEDTAVYYC
ARVHRWAYCINGVCFGAYSDYWGQGTLVTVSSASTKGPSVFPLAPSSKS
TSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAP
EFEGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPA
SIEKTISKAKGQPREPQVYTLPPCRDELTKNQVSLWCLVKGFYPSDIAV
EWESNGQPENNYKTTPPVLDSDGSFFLYSKLTVDKSRWQQGNVFSCSVL
HEALHSHYTQKSLSLSPGK

SEQ ID NO: 70 is the amino acid sequence defining the 1-57 derived light chain of the 2-17/1-57 antibody 1-57LC

EIVLTQSPGTLSLSPGERATLSCRASQSVSSSYLAWYQQKPGQAPRLLI
YGASSRATGIPDRFSGSGSGTDFTLTISRLEPEDFAVYYCQQYGSSPST
FGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC

The 2-17/2-15 bispecific antibody sequence can comprise:

SEQ ID NO: 71 is the amino acid sequence defining the 2-17 derived heavy chain of the 2-17/2-15 antibody 2-17HC-Hole-Cross

QVQLVQSGAEVKKPGSSVKVSCKASGGTFSSYAISWVRQAPGQGLEWMG
GNIPIFGTANYAQKFQGRVTITADESTSTAYMELSSLRSEDTAVYYCAR
GVGYRGVIPLNWFDPWGQGTVVTVSSASTAAPSVFIFPPSDEQLKSGTA
SVVCLLNNFYPREAKVQWKVDNALQSGNSQESVTEQDSKDSTYSLSSTL
TLSKADYEKHKVYACEVTHQGLSSPVTKSFNRGECDKTHTCPPCPAPEF
EGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPASI
EKTISKAKGQPREPQVCTLPPSRDELTKNQVSLSCAVKGFYPSDIAVEW
ESNGQPENNYKTTPPVLDSDGSFFLVSKLTVDKSRWQQGNVFSCSVLHE
ALHSHYTQKSLSLSPGK

SEQ ID NO: 72 is the amino acid sequence defining the 2-17 derived light chain of the 2-17/2-15 antibody 2-17VLCH1

EIVMTQSPATLSVSPGERATLSCRASQSVSSDLAWYQHKPGQAPRLLIY
GASTRATGIPVRFSGSGSGTEFTLTISSLQSEDFAVYYCQQYNNWPPFT
FGGGTKVEIKSSASTKGPSVFPLAPSSKSTSGGTAALGCLVKDYFPEPV
TVSWNSGALTSGVHTFPAVLQSSGLYSLSSVVTVPSSSLGTQTYICNVN
HKPSNTKVDKKVEPKSC

SEQ ID NO: 73 is the amino acid sequence defining the 2-15 derived heavy chain of the 2-17/2-15 antibody 2-15HC-Knob

QVQLVQSGAEVKKPGASVRVSCKASGYTFTGYYMHWVRQAPGQGLEWMG
WINPISDGTNYAQKFQGWVTMTRDTSISTVYMELSRLRSDDTAVYYCAR
GGSRCSGGNCYGWAYDAFDIWGQGTMITVSSASTKGPSVFPLAPSSKST
SGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPE
FEGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAS
IEKTISKAKGQPREPQVYTLPPCRDELTKNQVSLWCLVKGFYPSDIAVE
WESNGQPENNYKTTPPVLDSDGSFFLYSKLTVDKSRWQQGNVFSCSVLH
EALHSHYTQKSLSLSPGK

SEQ ID NO: 74 is the amino acid sequence defining the 2-15 derived light chain of the 2-17/2-15 antibody 2-15LC

QSALTQPASVSGSPGQSITISCTGTSSDVGGYNFVSWYQQHPGKAPKLM
IYDVSKRPSGVSNRFSGSKSGNTASLTISGLQAEDEADCYCSSYTSSST
FVFGTGTKVTVLGQPKAAPSVTLFPPSSEELQANKATLVCLISDFYPGA
VTVAWKADSSPVKAGVETTTPSKQSNNKYAASSYLSLTPEQWKSHRSYS
CQVTHEGSTVEKTVAPTECS

The 2-17/2-30 bispecific antibody sequence can comprise:

SEQ ID NO: 75 is the amino acid sequence defining the 2-17 derived heavy chain of the 2-17/2-30 antibody 2-17HC-Hole-Cross

QVQLVQSGAEVKKPGSSVKVSCKASGGTFSSYAISWVRQAPGQGLEWMG
GNIPIFGTANYAQKFQGRVTITADESTSTAYMELSSLRSEDTAVYYCAR
GVGYRGVIPLNWFDPWGQGTVVTVSSASTAAPSVFIFPPSDEQLKSGTA
SVVCLLNNFYPREAKVQWKVDNALQSGNSQESVTEQDSKDSTYSLSSTL
TLSKADYEKHKVYACEVTHQGLSSPVTKSFNRGECDKTHTCPPCPAPEF
EGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPASI
EKTISKAKGQPREPQVCTLPPSRDELTKNQVSLSCAVKGFYPSDIAVEW
ESNGQPENNYKTTPPVLDSDGSFFLVSKLTVDKSRWQQGNVFSCSVLHE
ALHSHYTQKSLSLSPGK

SEQ ID NO: 76 is the amino acid sequence defining the 2-17 derived light chain of the 2-17/2-30 antibody 2-17VLCH1

EIVMTQSPATLSVSPGERATLSCRASQSVSSDLAWYQHKPGQAPRLLIY
GASTRATGIPVRFSGSGSGTEFTLTISSLQSEDFAVYYCQQYNNWPPFT
FGGGTKVEIKSSASTKGPSVFPLAPSSKSTSGGTAALGCLVKDYFPEPV
TVSWNSGALTSGVHTFPAVLQSSGLYSLSSVVTVPSSSLGTQTYICNVN
HKPSNTKVDKKVEPKSC

SEQ ID NO: 77 is the amino acid sequence defining the 2-30 derived heavy chain of the 2-17/2-30 antibody 2-30 HC-Knob

QVQLVESGGGVVQPGRSLRLSCAASGFTFSSYTMHWVRQAPGKGLEWVA
AISYDGNNKYYADSVKGRFTISRDNSKNTLYLQMNSLRAEDTAVYYCAR
SILYGGGMDVWGQGTTVTVSSASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKKVEPKSCDKTHTCPPCPAPEFEGGPSVFLF
PPKPKDTLMISRTPEVTCVVVDVSHEDPEVKFNWYVDGVEVHNAKTKPR
EEQYNSTYRVVSVLTVLHQDWLNGKEYKCKVSNKALPASIEKTISKAKG
QPREPQVYTLPPCRDELTKNQVSLWCLVKGFYPSDIAVEWESNGQPENN
YKTTPPVLDSDGSFFLYSKLTVDKSRWQQGNVFSCSVLHEALHSHYTQK
SLSLSPGK

SEQ ID NO: 78 is the amino acid sequence defining the 2-30 derived light chain of the 2-17/2-30 antibody 2-30 LC

DIQLTQSPSSLSASVGDRVTITCRASQGISSYLAWYQQKPGQAPKLLIY
AASTLQSGVPSRFSGSGSGTDFTLTISSLQPEDFATYYCQQLNSYPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

The 2-17/4-20 bispecific antibody sequence can comprise:

SEQ ID NO: 79 is the amino acid sequence defining the 2-17 derived heavy chain of the 2-17/4-20 antibody 2-17HC-Hole-Cross

QVQLVQSGAEVKKPGSSVKVSCKASGGTFSSYAISWVRQAPGQGLEWMG
GNIPIFGTANYAQKFQGRVTITADESTSTAYMELSSLRSEDTAVYYCAR
GVGYRGVIPLNWFDPWGQGTVVTVSSASTAAPSVFIFPPSDEQLKSGTA
SVVCLLNNFYPREAKVQWKVDNALQSGNSQESVTEQDSKDSTYSLSSTL
TLSKADYEKHKVYACEVTHQGLSSPVTKSFNRGECDKTHTCPPCPAPEF
EGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPASI
EKTISKAKGQPREPQVCTLPPSRDELTKNQVSLSCAVKGFYPSDIAVEW
ESNGQPENNYKTTPPVLDSDGSFFLVSKLTVDKSRWQQGNVFSCSVLHE
ALHSHYTQKSLSLSPGK

SEQ ID NO: 80 is the amino acid sequence defining the 2-17 derived light chain of the 2-17/4-20 antibody 2-17VLCH1

EIVMTQSPATLSVSPGERATLSCRASQSVSSDLAWYQHKPGQAPRLLIY
GASTRATGIPVRFSGSGSGTEFTLTISSLQSEDFAVYYCQQYNNWPPFT
FGGGTKVEIKSSASTKGPSVFPLAPSSKSTSGGTAALGCLVKDYFPEPV
TVSWNSGALTSGVHTFPAVLQSSGLYSLSSVVTVPSSSLGTQTYICNVN
HKPSNTKVDKKVEPKSC

SEQ ID NO: 81 is the amino acid sequence defining the 4-20 derived heavy chain of the 2-17/4-20 antibody 4-20 HC-Knob

QVQLLQSGAEVKKPGASVKVSCKASGYSFTSYYMHWVRQAPGQGLEWMG
IINPSGGGTTYAQKFQGRVTMTRDTSTSTVYMELSSLRSEDTAVYYCAR
PGGGSYQEFDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPEFEGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPASIEKTISKAK
GQPREPQVYTLPPCRDELTKNQVSLWCLVKGFYPSDIAVEWESNGQPEN
NYKTTPPVLDSDGSFFLYSKLTVDKSRWQQGNVFSCSVLHEALHSHYTQ
KSLSLSPGK

SEQ ID NO: 82 is the amino acid sequence defining the 4-20 derived light chain of the 2-17/4-20 antibody 4-20 LC

DIQMTQSPSSLSASVGDRVTITCRASQSISSYLNWYQQKPGKAPKLLIS
AASSLQSGVPSRFSGSGSGTDFTLTISSLQPEDFATYYCQQSYNTLQVT
FGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC

The 5-24/1-57 bispecific antibody sequence can comprise:

SEQ ID NO: 83 is the amino acid sequence defining the 5-24 derived heavy chain of the 5-24/1-57 antibody 5-24HC-Hole-Cross

QVQLVESGGGVVQPGRSLRLSCAASGLTFSSYVMHWVRQAPGKGLDWVGV
IWYDGSKKYYADSVKGRFTISRDNSKNTLYLQMNSLRAEDTAVYYCARDP
RDYYDFWSGYDYYYGLDVWGQGTTVTVSSASTAAPSVFIFPPSDEQLKSG
TASVVCLLNNFYPREAKVQWKVDNALQSGNSQESVTEQDSKDSTYSLSST
LTLSKADYEKHKVYACEVTHQGLSSPVTKSFNRGECDKTHTCPPCPAPEF
EGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPASIEK
TISKAKGQPREPQVCTLPPSRDELTKNQVSLSCAVKGFYPSDIAVEWESN
GQPENNYKTTPPVLDSDGSFFLVSKLTVDKSRWQQGNVFSCSVLHEALHS
HYTQKSLSLSPGK

SEQ ID NO: 84 is the amino acid sequence defining the 5-24 derived light chain of the 5-24/1-57 antibody 5-24VLCH1

EIVLTQSPGILSLSPGERATLSCRASQSVSSSYLAWYQQKPGQAPRLLIY
GASSRATGIPDRFSGSGSGTDFTLTISRLEPEDFAVYYCQQYGSSGALTF
GGGTKVEIKSSASTKGPSVFPLAPSSKSTSGGTAALGCLVKDYFPEPVTV
SWNSGALTSGVHTFPAVLQSSGLYSLSSVVTVPSSSLGTQTYICNVNHKP
SNTKVDKKVEPKSC

SEQ ID NO: 85 is the amino acid sequence defining the 1-57 derived heavy chain of the 5-24/1-57 antibody 1-57HC-Knob

EVHLVESGGGLVQPGGSLRLSCAASGFTFSDHYMDWVRQAPGKGLEWVGR
TRNKANSYTTEYAASVKGRFTISRDDAKNSLYLQMNSLKTEDTAVYYCAR
VHRWAYCINGVCFGAYSDYWGQGTLVTVSSASTKGPSVFPLAPSSKSTSG
GTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEFEGG
PSVFLFPPKPKDTLMISRTPEVTCVVVDVSHEDPEVKFNWYVDGVEVHNA
KTKPREEQYNSTYRVVSVLTVLHQDWLNGKEYKCKVSNKALPASIEKTIS
KAKGQPREPQVYTLPPCRDELTKNQVSLWCLVKGFYPSDIAVEWESNGQP
ENNYKTTPPVLDSDGSFFLYSKLTVDKSRWQQGNVFSCSVLHEALHSHYT
QKSLSLSPGK

SEQ ID NO: 86 is the amino acid sequence defining the 1-57 derived light chain of the 5-24/1-57 antibody 1-57LC

EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPSTFG
QGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC

The 5-24/2-15 bispecific antibody sequence can comprise:

SEQ ID NO: 87 is the amino acid sequence defining the 5-24 derived heavy chain of the 5-24/2-15 antibody 5-24HC-Hole-Cross

QVQLVESGGGVVQPGRSLRLSCAASGLTFSSYVMHWVRQAPGKGLDWVGV
IWYDGSKKYYADSVKGRFTISRDNSKNTLYLQMNSLRAEDTAVYYCARDP
RDYYDFWSGYDYYYGLDVWGQGTTVTVSSASTAAPSVFIFPPSDEQLKSG
TASVVCLLNNFYPREAKVQWKVDNALQSGNSQESVTEQDSKDSTYSLSST
LTLSKADYEKHKVYACEVTHQGLSSPVTKSFNRGECDKTHTCPPCPAPEF
EGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPASIEK
TISKAKGQPREPQVCTLPPSRDELTKNQVSLSCAVKGFYPSDIAVEWESN
GQPENNYKTTPPVLDSDGSFFLVSKLTVDKSRWQQGNVFSCSVLHEALHS
HYTQKSLSLSPGK

SEQ ID NO: 88 is the amino acid sequence defining the 5-24 derived light chain of the 5-24/2-15 antibody 5-24VLCH1

EIVLTQSPGILSLSPGERATLSCRASQSVSSSYLAWYQQKPGQAPRLLIY
GASSRATGIPDRFSGSGSGTDFTLTISRLEPEDFAVYYCQQYGSSGALTF
GGGTKVEIKSSASTKGPSVFPLAPSSKSTSGGTAALGCLVKDYFPEPVTV
SWNSGALTSGVHTFPAVLQSSGLYSLSSVVTVPSSSLGTQTYICNVNHKP
SNTKVDKKVEPKSC

SEQ ID NO: 89 is the amino acid sequence defining the 2-15 derived heavy chain of the 5-24/2-15 antibody 2-15HC-Knob

QVQLVQSGAEVKKPGASVRVSCKASGYTFTGYYMHWVRQAPGQGLEWMGW
INPISDGTNYAQKFQGWVTMTRDTSISTVYMELSRLRSDDTAVYYCARGG
SRCSGGNCYGWAYDAFDIWGQGTMITVSSASTKGPSVFPLAPSSKSTSGG
TAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEFEGGP
SVFLFPPKPKDTLMISRTPEVTCVVVDVSHEDPEVKFNWYVDGVEVHNAK
TKPREEQYNSTYRVVSVLTVLHQDWLNGKEYKCKVSNKALPASIEKTISK
AKGQPREPQVYTLPPCRDELTKNQVSLWCLVKGFYPSDIAVEWESNGQPE
NNYKTTPPVLDSDGSFFLYSKLTVDKSRWQQGNVFSCSVLHEALHSHYTQ
KSLSLSPGK

SEQ ID NO: 90 is the amino acid sequence defining the 2-15 derived light chain of the 5-24/2-15 antibody 2-15LC

QSALTQPASVSGSPGQSITISCTGTSSDVGGYNFVSWYQQHPGKAPKLMI
YDVSKRPSGVSNRFSGSKSGNTASLTISGLQAEDEADCYCSSYTSSSTFV
FGTGTKVTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECS

The 5-24/4-20 bispecific antibody sequence can comprise:

SEQ ID NO: 91 is the amino acid sequence defining the 5-24 derived heavy chain of the 5-24/4-20 antibody 5-24HC-Hole-Cross

QVQLVESGGGVVQPGRSLRLSCAASGLTFSSYVMHWVRQAPGKGLDWVGV
IWYDGSKKYYADSVKGRFTISRDNSKNTLYLQMNSLRAEDTAVYYCARDP
RDYYDFWSGYDYYYGLDVWGQGTTVTVSSASTAAPSVFIFPPSDEQLKSG
TASVVCLLNNFYPREAKVQWKVDNALQSGNSQESVTEQDSKDSTYSLSST
LTLSKADYEKHKVYACEVTHQGLSSPVTKSFNRGECDKTHTCPPCPAPEF
EGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPASIEK
TISKAKGQPREPQVCTLPPSRDELTKNQVSLSCAVKGFYPSDIAVEWESN
GQPENNYKTTPPVLDSDGSFFLVSKLTVDKSRWQQGNVFSCSVLHEALHS
HYTQKSLSLSPGK

SEQ ID NO: 92 is the amino acid sequence defining the 5-24 derived light chain of the 5-24/4-20 antibody 5-24VLCH1

EIVLTQSPGILSLSPGERATLSCRASQSVSSSYLAWYQQKPGQAPRLLIY
GASSRATGIPDRFSGSGSGTDFTLTISRLEPEDFAVYYCQQYGSSGALTF
GGGTKVEIKSSASTKGPSVFPLAPSSKSTSGGTAALGCLVKDYFPEPVTV
SWNSGALTSGVHTFPAVLQSSGLYSLSSVVTVPSSSLGTQTYICNVNHKP
SNTKVDKKVEPKSC

SEQ ID NO: 93 is the amino acid sequence defining the 4-20 derived heavy chain of the 5-24/4-20 antibody 4-20HC-Knob

QVQLLQSGAEVKKPGASVKVSCKASGYSFTSYYMHWVRQAPGQGLEWMGI
INPSGGGTTYAQKFQGRVTMTRDTSTSTVYMELSSLRSEDTAVYYCARPG
GGSYQEFDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPEFEGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPASIEKTISKAKGQPREPQ
VYTLPPCRDELTKNQVSLWCLVKGFYPSDIAVEWESNGQPENNYKTTPPV
LDSDGSFFLYSKLTVDKSRWQQGNVFSCSVLHEALHSHYTQKSLSLSPGK

SEQ ID NO: 94 is the amino acid sequence defining the 4-20 derived light chain of the 5-24/4-20 antibody 4-20LC

DIQMTQSPSSLSASVGDRVTITCRASQSISSYLNWYQQKPGKAPKLLISA
ASSLQSGVPSRFSGSGSGTDFTLTISSLQPEDFATYYCQQSYNTLQVTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC

The 1-20/1-68 bispecific antibody sequence can comprise:

SEQ ID NO: 95 is the amino acid sequence defining the 1-20 derived heavy chain of the 1-20/1-68 antibody 1-20HC-Hole-Cross

EVQLVESGGGLIQPGGSLRLSCAASGLTVSSNYMSWVRQAPGKGLEWVSV
IYSGGSTFYADSVKGRFTISRDNSKNTLYLQMNSLRAEDTAVYYCARDLF
YYGMDVWGQGTTVTVSSASTAAPSVFIFPPSDEQLKSGTASVVCLLNNFY
PREAKVQWKVDNALQSGNSQESVTEQDSKDSTYSLSSTLTLSKADYEKHK
VYACEVTHQGLSSPVTKSFNRGECDKTHTCPPCPAPEFEGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPASIEKTISKAKGQPREP
QVCTLPPSRDELTKNQVSLSCAVKGFYPSDIAVEWESNGQPENNYKTTPP
VLDSDGSFFLVSKLTVDKSRWQQGNVFSCSVLHEALHSHYTQKSLSLSPG
K

SEQ ID NO: 96 is the amino acid sequence defining the 1-20 derived light chain of the 1-20/1-68 antibody 1-20VLCH1

DIQLTQSPSFLSASVGDRVTITCRASQGISSYLAWYQQKPGKAPKLLIYA
ASTLQSGVPSRFSGSGSGTEFTLTISSLQPEDFATYYCQQLNSYPCFGPG
TKVDIKSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNT
KVDKKVEPKSC

SEQ ID NO: 97 is the amino acid sequence defining the 1-68 derived heavy chain of the 1-20/1-68 antibody 1-68HC-Knob

QVQLVQSGAEVKKPGASVKVSCKVSGYTLIELSMHWVRQAPGKGLEWMGG
FDPEDAETIYAQKFQGRVTMTEDTSTDTAYMELSSLRSEDTAVYYCATGW
AVAGSSDVWYYYYGMDVWGQGTTVTVSSASTKGPSVFPLAPSSKSTSGGT
AALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPEFEGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPASIEKTISKA
KGQPREPQVYTLPPCRDELTKNQVSLWCLVKGFYPSDIAVEWESNGQPEN
NYKTTPPVLDSDGSFFLYSKLTVDKSRWQQGNVFSCSVLHEALHSHYTQK
SLSLSPGK

SEQ ID NO: 98 is the amino acid sequence defining the 1-68 derived light chain of the 1-20/1-68 antibody 1-68LC

QSALTQPPSVSGSPGQSVTISCTGTSSDVGSYNRVSWYQQPPGTAPKLMI
YEVSNRPSGVPDRFSGSKSGNTASLTISGLQAEDEADYYCSSYTSSSTYV
FGTGTKVTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECS

The 1-20/2-17 bispecific antibody sequence can comprise:

SEQ ID NO: 99 is the amino acid sequence defining the 1-20 derived heavy chain of the 1-20/2-17 antibody 1-20HC-Hole-Cross

EVQLVESGGGLIQPGGSLRLSCAASGLTVSSNYMSWVRQAPGKGLEWVSV
IYSGGSTFYADSVKGRFTISRDNSKNTLYLQMNSLRAEDTAVYYCARDLF
YYGMDVWGQGTTVTVSSASTAAPSVFIFPPSDEQLKSGTASVVCLLNNFY
PREAKVQWKVDNALQSGNSQESVTEQDSKDSTYSLSSTLTLSKADYEKHK
VYACEVTHQGLSSPVTKSFNRGECDKTHTCPPCPAPEFEGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPASIEKTISKAKGQPREP
QVCTLPPSRDELTKNQVSLSCAVKGFYPSDIAVEWESNGQPENNYKTTPP
VLDSDGSFFLVSKLTVDKSRWQQGNVFSCSVLHEALHSHYTQKSLSLSPG
K

SEQ ID NO: 100 is the amino acid sequence defining the 1-20 derived light chain of the 1-20/2-17 antibody 1-20VLCH1

DIQLTQSPSFLSASVGDRVTITCRASQGISSYLAWYQQKPGKAPKLLIYA
ASTLQSGVPSRFSGSGSGTEFTLTISSLQPEDFATYYCQQLNSYPCFGPG
TKVDIKSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNT
KVDKKVEPKSC

SEQ ID NO: 101 is the amino acid sequence defining the 2-17 derived heavy chain of the 1-20/2-17 antibody 2-17HC-Knob

QVQLVQSGAEVKKPGSSVKVSCKASGGTFSSYAISWVRQAPGQGLEWMGG
NIPIFGTANYAQKFQGRVTITADESTSTAYMELSSLRSEDTAVYYCARGV
GYRGVIPLNWFDPWGQGTVVTVSSASTKGPSVFPLAPSSKSTSGGTAALG
CLVKDYFPEPVTVSWNSGALTSGVHTFPAVLQSSGLYSLSSVVTVPSSSL
GTQTYICNVNHKPSNTKVDKKVEPKSCDKTHTCPPCPAPEFEGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPASIEKTISKAKGQP
REPQVYTLPPCRDELTKNQVSLWCLVKGFYPSDIAVEWESNGQPENNYKT
TPPVLDSDGSFFLYSKLTVDKSRWQQGNVFSCSVLHEALHSHYTQKSLSL
SPGK

SEQ ID NO: 102 is the amino acid sequence defining the 2-17 derived light chain of the 1-20/2-17 antibody 2-17LC

EIVMTQSPATLSVSPGERATLSCRASQSVSSDLAWYQHKPGQAPRLLIYG
ASTRATGIPVRFSGSGSGTEFTLTISSLQSEDFAVYYCQQYNNWPPFTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC

The 1-20/4-18 bispecific antibody sequence can comprise:

SEQ ID NO: 103 is the amino acid sequence defining the 1-20 derived heavy chain of the 1-20/4-18 antibody 1-20HC-Hole-Cross

EVQLVESGGGLIQPGGSLRLSCAASGLTVSSNYMSWVRQAPGKGLEWVSV
IYSGGSTFYADSVKGRFTISRDNSKNTLYLQMNSLRAEDTAVYYCARDLF
YYGMDVWGQGTTVTVSSASTAAPSVFIFPPSDEQLKSGTASVVCLLNNFY
PREAKVQWKVDNALQSGNSQESVTEQDSKDSTYSLSSTLTLSKADYEKHK
VYACEVTHQGLSSPVTKSFNRGECDKTHTCPPCPAPEFEGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPASIEKTISKAKGQPREP
QVCTLPPSRDELTKNQVSLSCAVKGFYPSDIAVEWESNGQPENNYKTTPP
VLDSDGSFFLVSKLTVDKSRWQQGNVFSCSVLHEALHSHYTQKSLSLSPG
K

SEQ ID NO: 104 is the amino acid sequence defining the 1-20 derived light chain of the 1-20/4-18 antibody 1-20VLCH1

DIQLTQSPSFLSASVGDRVTITCRASQGISSYLAWYQQKPGKAPKLLIYA
ASTLQSGVPSRFSGSGSGTEFTLTISSLQPEDFATYYCQQLNSYPCFGPG
TKVDIKSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNT
KVDKKVEPKSC

SEQ ID NO: 105 is the amino acid sequence defining the 4-18 derived heavy chain of the 1-20/4-18 antibody 4-18HC-Knob

QVQLVESGGGVVQPGRSLRLSCAASGFTFSSYGMHWVRQAPGKGLEWVAV
ISYDGSNKHYADSVKGRFTISRDNSKNTLYLQMNSLRAEDTAVYYCAKDS
GYNYGYSWFDPWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPEFEGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPASIEKTISKAKGQPRE
PQVYTLPPCRDELTKNQVSLWCLVKGFYPSDIAVEWESNGQPENNYKTTP
PVLDSDGSFFLYSKLTVDKSRWQQGNVFSCSVLHEALHSHYTQKSLSLSP
GK

SEQ ID NO: 106 is the amino acid sequence defining the 4-18 derived light chain of the 1-20/4-18 antibody 4-18LC

SYELTQPPSVSVSPGQTARITCSADALAKQYAYWYQQKPGQAPVLVIYKD
SERPSGIPERFSGSSSGTTVTLTISGVQAEDEADYYCQSTDNSGTYPNWV
FGGGTKLTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECS

The 1-20/5-24 bispecific antibody sequence can comprise:

SEQ ID NO: 107 is the amino acid sequence defining the 1-20 derived heavy chain of the 1-20/5-24 antibody 1-20HC-Hole-Cross

EVQLVESGGGLIQPGGSLRLSCAASGLTVSSNYMSWVRQAPGKGLEWVSV
IYSGGSTFYADSVKGRFTISRDNSKNTLYLQMNSLRAEDTAVYYCARDLF
YYGMDVWGQGTTVTVSSASTAAPSVFIFPPSDEQLKSGTASVVCLLNNFY
PREAKVQWKVDNALQSGNSQESVTEQDSKDSTYSLSSTLTLSKADYEKHK
VYACEVTHQGLSSPVTKSFNRGECDKTHTCPPCPAPEFEGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPASIEKTISKAKGQPREP
QVCTLPPSRDELTKNQVSLSCAVKGFYPSDIAVEWESNGQPENNYKTTPP
VLDSDGSFFLVSKLTVDKSRWQQGNVFSCSVLHEALHSHYTQKSLSLSPG
K

SEQ ID NO: 108 is the amino acid sequence defining the 1-20 derived light chain of the 1-20/5-24 antibody 1-20VLCH1

DIQLTQSPSFLSASVGDRVTITCRASQGISSYLAWYQQKPGKAPKLLIYA
ASTLQSGVPSRFSGSGSGTEFTLTISSLQPEDFATYYCQQLNSYPCFGPG
TKVDIKSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNT
KVDKKVEPKSC

SEQ ID NO: 109 is the amino acid sequence defining the 5-24 derived heavy chain of the 1-20/5-24 antibody 5-24HC-Knob

QVQLVESGGGVVQPGRSLRLSCAASGLTFSSYVMHWVRQAPGKGLDWVGV
IWYDGSKKYYADSVKGRFTISRDNSKNTLYLQMNSLRAEDTAVYYCARDP
RDYYDFWSGYDYYYGLDVWGQGTTVTVSSASTKGPSVFPLAPSSKSTSGG
TAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEFEGGP
SVFLFPPKPKDTLMISRTPEVTCVVVDVSHEDPEVKFNWYVDGVEVHNAK
TKPREEQYNSTYRVVSVLTVLHQDWLNGKEYKCKVSNKALPASIEKTISK
AKGQPREPQVYTLPPCRDELTKNQVSLWCLVKGFYPSDIAVEWESNGQPE
NNYKTTPPVLDSDGSFFLYSKLTVDKSRWQQGNVFSCSVLHEALHSHYTQ
KSLSLSPGK

SEQ ID NO: 110 is the amino acid sequence defining the 5-24 derived light chain of the 1-20/5-24 antibody 5-24LC

EIVLTQSPGILSLSPGERATLSCRASQSVSSSYLAWYQQKPGQAPRLLIY
GASSRATGIPDRFSGSGSGTDFTLTISRLEPEDFAVYYCQQYGSSGALTF
GGGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVTH
QGLSSPVTKSFNRGEC

Bispecific antibodies with both LALA (TM) and LS mutations:

The 2-7/4-8(39/51/57) bispecific antibody sequence can comprise:

SEQ ID NO: 111 is the amino acid sequence defining the 2-7 derived heavy chain of the 2-7/4-8(39/51/57) antibody 2-7HC-Hole-Cross

QITLKESGPTLVKPTQTLTLTCTFSGFSLSTSGVGVGWIRQPPGKALEWL
ALIYWDDDKRYSPSLKSRLTITKDTSKNQVVLTMTNMDPVDTATYYCAHH
KIERIFDYWGQGTLVTVSSASTAAPSVFIFPPSDEQLKSGTASVVCLLNN
FYPREAKVQWKVDNALQSGNSQESVTEQDSKDSTYSLSSTLTLSKADYEK
HKVYACEVTHQGLSSPVTKSFNRGECDKTHTCPPCPAPEFEGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPASIEKTISKAKGQPR
EPQVCTLPPSRDELTKNQVSLSCAVKGFYPSDIAVEWESNGQPENNYKTT
PPVLDSDGSFFLVSKLTVDKSRWQQGNVFSCSVLHEALHSHYTQKSLSLS
PGK

SEQ ID NO: 112 is the amino acid sequence defining the 2-7 derived light chain of the 2-7/4-8(39/51/57) antibody 2-7VLCH1

QSALAQPASVSGSPGQSITISCTGTSSDVGAYNYVSWYQQHPGKAPKLMI
YDVSKRPSGVSNRFSGSKSGNTASLTISGLQAEDEADYYCSSYTTSSTVF
GGGTKLTVLSSASTKGPSVFPLAPSSKSTSGGTAALGCLVKDYFPEPVTV
SWNSGALTSGVHTFPAVLQSSGLYSLSSVVTVPSSSLGTQTYICNVNHKP
SNTKVDKKVEPKSC

SEQ ID NO: 113 is the amino acid sequence defining the 4-8(39/51/57) derived heavy chain of the 2-7/4-8(39/51/57) antibody 4-8(39/51/57) HC-Knob

QVQLVQSGAEVKKAGSSVKVSCKASGGTFSSHTITWVRLAPGQGLEWMGR
NIPILGTANYAQKFQGRVTITADKSTSTAYMELSSLRSEDTAVYYCASLQ
TVDTAIEKYYGMDVWGQGTTVTVSSASTKGPSVFPLAPSSKSTSGGTAAL
GCLVKDYFPEPVTVSWNSGALTSGVHTFPAVLQSSGLYSLSSVVTVPSSS
LGTQTYICNVNHKPSNTKVDKKVEPKSCDKTHTCPPCPAPEFEGGPSVFL
FPPKPKDTLMISRTPEVTCVVVDVSHEDPEVKFNWYVDGVEVHNAKTKPR
EEQYNSTYRVVSVLTVLHQDWLNGKEYKCKVSNKALPASIEKTISKAKGQ
PREPQVYTLPPCRDELTKNQVSLWCLVKGFYPSDIAVEWESNGQPENNYK
TTPPVLDSDGSFFLYSKLTVDKSRWQQGNVFSCSVLHEALHSHYTQKSLS
LSPGK

SEQ ID NO: 114 is the amino acid sequence defining the 4-8(39/51/57) derived light chain of the 2-7/4-8(39/51/57) antibody 4-8LC

SYELTQPPSVSVSPGQTASITCSGDKLGDKYACWYQQKPGQSPVLVIYQD
NKRPSGIPERFSGSNSGNTATLTISGTQAMDEADYYCQAWDSSTAVFGGG
TKLTVLGQPKAAPSVTLFPPSSEELQANKATLVCLISDFYPGAVTVAWKA
DSSPVKAGVETTTPSKQSNNKYAASSYLSLTPEQWKSHRSYSCQVTHEGS
TVEKTVAPTECS

The 2-17/2-36 bispecific antibody sequence can comprise:

SEQ ID NO: 115 is the amino acid sequence defining the 2-17 derived heavy chain of the 2-17/2-36 antibody 2-17HC-Hole-Cross

QVQLVQSGAEVKKPGSSVKVSCKASGGTFSSYAISWVRQAPGQGLEWMGG
NIPIFGTANYAQKFQGRVTITADESTSTAYMELSSLRSEDTAVYYCARGV
GYRGVIPLNWFDPWGQGTVVTVSSASTAAPSVFIFPPSDEQLKSGTASVV
CLLNNFYPREAKVQWKVDNALQSGNSQESVTEQDSKDSTYSLSSTLTLSK
ADYEKHKVYACEVTHQGLSSPVTKSFNRGECDKTHTCPPCPAPEFEGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPASIEKTISKA
KGQPREPQVCTLPPSRDELTKNQVSLSCAVKGFYPSDIAVEWESNGQPEN
NYKTTPPVLDSDGSFFLVSKLTVDKSRWQQGNVFSCSVLHEALHSHYTQK
SLSLSPGK

SEQ ID NO: 116 is the amino acid sequence defining the 2-17 derived light chain of the 2-17/2-36 antibody 2-17VLCH1

EIVMTQSPATLSVSPGERATLSCRASQSVSSDLAWYQHKPGQAPRLLIYG
ASTRATGIPVRFSGSGSGTEFTLTISSLQSEDFAVYYCQQYNNWPPFTFG
GGTKVEIKSSASTKGPSVFPLAPSSKSTSGGTAALGCLVKDYFPEPVTVS
WNSGALTSGVHTFPAVLQSSGLYSLSSVVTVPSSSLGTQTYICNVNHKPS
NTKVDKKVEPKSC

SEQ ID NO: 117 is the amino acid sequence defining the 2-36 derived heavy chain of the 2-17/2-36 antibody 2-36HC-Knob

QVQLQESGPGLVKPSETLSLTCTVSGGSVSSSNYYWSWIRQPPGKGLEWI
GYMYYSGSTKYNPSLKSRVTISVDTSKNQFSLKLSSVTAADTAVYYCARE
VYYYDRSGYYASDGFDIWGQGTMVTVSSASTKGPSVFPLAPSSKSTSGGT
AALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPEFEGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPASIEKTISKA
KGQPREPQVYTLPPCRDELTKNQVSLWCLVKGFYPSDIAVEWESNGQPEN
NYKTTPPVLDSDGSFFLYSKLTVDKSRWQQGNVFSCSVLHEALHSHYTQK
SLSLSPGK

SEQ ID NO: 118 is the amino acid sequence defining the 2-36 derived light chain of the 2-17/2-36 antibody 2-36LC

EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPQTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC

The 2-36/1-68 bispecific antibody sequence can comprise:

SEQ ID NO: 119 is the amino acid sequence defining the 2-36 derived heavy chain of the 2-36/1-68 antibody 2-36HC-Hole-Cross

QVQLQESGPGLVKPSETLSLTCTVSGGSVSSSNYYWSWIRQPPGKGLEWI
GYMYYSGSTKYNPSLKSRVTISVDTSKNQFSLKLSSVTAADTAVYYCARE
VYYYDRSGYYASDGFDIWGQGTMVTVSSASTAAPSVFIFPPSDEQLKSGT
ASVVCLLNNFYPREAKVQWKVDNALQSGNSQESVTEQDSKDSTYSLSSTL
TLSKADYEKHKVYACEVTHQGLSSPVTKSFNRGECDKTHTCPPCPAPEFE
GGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPASIEKT
ISKAKGQPREPQVCTLPPSRDELTKNQVSLSCAVKGFYPSDIAVEWESNG
QPENNYKTTPPVLDSDGSFFLVSKLTVDKSRWQQGNVFSCSVLHEALHSH
YTQKSLSLSPGK

SEQ ID NO: 120 is the amino acid sequence defining the 2-36 derived light chain of the 2-36/1-68 antibody 2-36VLCH1

EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPQTFG
QGTKVEIKSSASTKGPSVFPLAPSSKSTSGGTAALGCLVKDYFPEPVTVS
WNSGALTSGVHTFPAVLQSSGLYSLSSVVTVPSSSLGTQTYICNVNHKPS
NTKVDKKVEPKSC

SEQ ID NO: 121 is the amino acid sequence defining the 1-68 derived heavy chain of the 2-36/1-68 antibody 1-68HC-Knob

QVQLVQSGAEVKKPGASVKVSCKVSGYTLIELSMHWVRQAPGKGLEWMGG
FDPEDAETIYAQKFQGRVTMTEDTSTDTAYMELSSLRSEDTAVYYCATGW
AVAGSSDVWYYYYGMDVWGQGTTVTVSSASTKGPSVFPLAPSSKSTSGGT
AALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPEFEGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPASIEKTISKA
KGQPREPQVYTLPPCRDELTKNQVSLWCLVKGFYPSDIAVEWESNGQPEN
NYKTTPPVLDSDGSFFLYSKLTVDKSRWQQGNVFSCSVLHEALHSHYTQK
SLSLSPGK

SEQ ID NO: 122 is the amino acid sequence defining the 1-68 derived light chain of the 2-36/1-68 antibody 1-68LC

QSALTQPPSVSGSPGQSVTISCTGTSSDVGSYNRVSWYQQPPGTAPKLMI
YEVSNRPSGVPDRFSGSKSGNTASLTISGLQAEDEADYYCSSYTSSSTYV
FGTGTKVTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECS

The 2-36/4-18 bispecific antibody sequence can comprise:

SEQ ID NO: 123 is the amino acid sequence defining the 2-36 derived heavy chain of the 2-36/4-18 antibody 2-36HC-Hole-Cross

QVQLQESGPGLVKPSETLSLTCTVSGGSVSSSNYYWSWIRQPPGKGLEWI
GYMYYSGSTKYNPSLKSRVTISVDTSKNQFSLKLSSVTAADTAVYYCARE
VYYYDRSGYYASDGFDIWGQGTMVTVSSASTAAPSVFIFPPSDEQLKSGT
ASVVCLLNNFYPREAKVQWKVDNALQSGNSQESVTEQDSKDSTYSLSSTL
TLSKADYEKHKVYACEVTHQGLSSPVTKSFNRGECDKTHTCPPCPAPEFE
GGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPASIEKT
ISKAKGQPREPQVCTLPPSRDELTKNQVSLSCAVKGFYPSDIAVEWESNG
QPENNYKTTPPVLDSDGSFFLVSKLTVDKSRWQQGNVFSCSVLHEALHSH
YTQKSLSLSPGK

SEQ ID NO: 124 is the amino acid sequence defining the 2-36 derived light chain of the 2-36/4-18 antibody 2-36VLCH1

EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPQTFG
QGTKVEIKSSASTKGPSVFPLAPSSKSTSGGTAALGCLVKDYFPEPVTVS
WNSGALTSGVHTFPAVLQSSGLYSLSSVVTVPSSSLGTQTYICNVNHKPS
NTKVDKKVEPKSC

SEQ ID NO: 125 is the amino acid sequence defining the 4-18 derived heavy chain of the 2-36/4-18 antibody 4-18HC-Knob

QVQLVESGGGVVQPGRSLRLSCAASGFTFSSYGMHWVRQAPGKGLEWVAV
ISYDGSNKHYADSVKGRFTISRDNSKNTLYLQMNSLRAEDTAVYYCAKDS
GYNYGYSWFDPWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPEFEGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPASIEKTISKAKGQPRE
PQVYTLPPCRDELTKNQVSLWCLVKGFYPSDIAVEWESNGQPENNYKTTP
PVLDSDGSFFLYSKLTVDKSRWQQGNVFSCSVLHEALHSHYTQKSLSLSP
GK

SEQ ID NO: 126 is the amino acid sequence defining the 4-18 derived light chain of the 2-36/4-18 antibody 4-18LC

SYELTQPPSVSVSPGQTARITCSADALAKQYAYWYQQKPGQAPVLVIYKD
SERPSGIPERFSGSSSGTTVTLTISGVQAEDEADYYCQSTDNSGTYPNWV
FGGGTKLTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECS

The 2-30/1-68 bispecific antibody sequence can comprise:

SEQ ID NO: 127 is the amino acid sequence defining the 2-30 derived heavy chain of the 2-30/1-68 antibody 2-30HC-Hole-Cross

QVQLVESGGGVVQPGRSLRLSCAASGFTFSSYTMHWVRQAPGKGLEWVAA
ISYDGNNKYYADSVKGRFTISRDNSKNTLYLQMNSLRAEDTAVYYCARSI
LYGGGMDVWGQGTTVTVSSASTAAPSVFIFPPSDEQLKSGTASVVCLLNN
FYPREAKVQWKVDNALQSGNSQESVTEQDSKDSTYSLSSTLTLSKADYEK
HKVYACEVTHQGLSSPVTKSFNRGECDKTHTCPPCPAPEFEGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPASIEKTISKAKGQPR
EPQVCTLPPSRDELTKNQVSLSCAVKGFYPSDIAVEWESNGQPENNYKTT
PPVLDSDGSFFLVSKLTVDKSRWQQGNVFSCSVLHEALHSHYTQKSLSLS
PGK

SEQ ID NO: 128 is the amino acid sequence defining the 2-30 derived light chain of the 2-30/1-68 antibody 2-30VLCH1

DIQLTQSPSSLSASVGDRVTITCRASQGISSYLAWYQQKPGQAPKLLIYA
ASTLQSGVPSRFSGSGSGTDFTLTISSLQPEDFATYYCQQLNSYPLTFGG
GTKVEIKSSASTKGPSVFPLAPSSKSTSGGTAALGCLVKDYFPEPVTVSW
NSGALTSGVHTFPAVLQSSGLYSLSSVVTVPSSSLGTQTYICNVNHKPSN
TKVDKKVEPKSC

SEQ ID NO: 129 is the amino acid sequence defining the 1-68 derived heavy chain of the 2-30/1-68 antibody 1-68HC-Knob

QVQLVQSGAEVKKPGASVKVSCKVSGYTLIELSMHWVRQAPGKGLEWMGG
FDPEDAETIYAQKFQGRVTMTEDTSTDTAYMELSSLRSEDTAVYYCATGW
AVAGSSDVWYYYYGMDVWGQGTTVTVSSASTKGPSVFPLAPSSKSTSGGT
AALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPEFEGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPASIEKTISKA
KGQPREPQVYTLPPCRDELTKNQVSLWCLVKGFYPSDIAVEWESNGQPEN
NYKTTPPVLDSDGSFFLYSKLTVDKSRWQQGNVFSCSVLHEALHSHYTQK
SLSLSPGK

SEQ ID NO: 130 is the amino acid sequence defining the 1-68 derived light chain of the 2-30/1-68 antibody 1-68LC

QSALTQPPSVSGSPGQSVTISCTGTSSDVGSYNRVSWYQQPPGTAPKLMI
YEVSNRPSGVPDRFSGSKSGNTASLTISGLQAEDEADYYCSSYTSSSTYV
FGTGTKVTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECS

The 2-30/4-18 bispecific antibody can comprise:

SEQ ID NO: 131 is the amino acid sequence defining the 2-30 derived heavy chain of the 2-30/4-18 antibody 2-30HC-Hole-Cross

QVQLVESGGGVVQPGRSLRLSCAASGFTFSSYTMHWVRQAPGKGLEWVAA
ISYDGNNKYYADSVKGRFTISRDNSKNTLYLQMNSLRAEDTAVYYCARSI
LYGGGMDVWGQGTTVTVSSASTAAPSVFIFPPSDEQLKSGTASVVCLLNN
FYPREAKVQWKVDNALQSGNSQESVTEQDSKDSTYSLSSTLTLSKADYEK
HKVYACEVTHQGLSSPVTKSFNRGECDKTHTCPPCPAPEFEGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPASIEKTISKAKGQPR
EPQVCTLPPSRDELTKNQVSLSCAVKGFYPSDIAVEWESNGQPENNYKTT
PPVLDSDGSFFLVSKLTVDKSRWQQGNVFSCSVLHEALHSHYTQKSLSLS
PGK

SEQ ID NO: 132 is the amino acid sequence defining the 2-30 derived light chain of the 2-30/4-18 antibody 2-30VLCH1

DIQLTQSPSSLSASVGDRVTITCRASQGISSYLAWYQQKPGQAPKLLIYA
ASTLQSGVPSRFSGSGSGTDFTLTISSLQPEDFATYYCQQLNSYPLTFGG
GTKVEIKSSASTKGPSVFPLAPSSKSTSGGTAALGCLVKDYFPEPVTVSW
NSGALTSGVHTFPAVLQSSGLYSLSSVVTVPSSSLGTQTYICNVNHKPSN
TKVDKKVEPKSC

SEQ ID NO: 133 is the amino acid sequence defining the 4-18 derived heavy chain of the 2-30/4-18 antibody 4-18HC-Knob

QVQLVESGGGVVQPGRSLRLSCAASGFTFSSYGMHWVRQAPGKGLEWVAV
ISYDGSNKHYADSVKGRFTISRDNSKNTLYLQMNSLRAEDTAVYYCAKDS
GYNYGYSWFDPWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPEFEGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPASIEKTISKAKGQPRE
PQVYTLPPCRDELTKNQVSLWCLVKGFYPSDIAVEWESNGQPENNYKTTP
PVLDSDGSFFLYSKLTVDKSRWQQGNVFSCSVLHEALHSHYTQKSLSLSP
GK

SEQ ID NO: 134 is the amino acid sequence defining the 4-18 derived light chain of the 2-30/4-18 antibody 4-18LC

SYELTQPPSVSVSPGQTARITCSADALAKQYAYWYQQKPGQAPVLVIYKD
SERPSGIPERFSGSSSGTTVTLTISGVQAEDEADYYCQSTDNSGTYPNWV
FGGGTKLTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECS

The 2-7/4-8(39/51) bispecific antibody sequence can comprise:

SEQ ID NO: 135 is the amino acid sequence defining the 2-7 derived heavy chain of the 2-7/4-8(39/51) antibody 2-7HC-Hole-Cross

QITLKESGPTLVKPTQTLTLTCTFSGFSLSTSGVGVGWIRQPPGKALEWL
ALIYWDDDKRYSPSLKSRLTITKDTSKNQVVLTMTNMDPVDTATYYCAHH
KIERIFDYWGQGTLVTVSSASTAAPSVFIFPPSDEQLKSGTASVVCLLNN
FYPREAKVQWKVDNALQSGNSQESVTEQDSKDSTYSLSSTLTLSKADYEK
HKVYACEVTHQGLSSPVTKSFNRGECDKTHTCPPCPAPEFEGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPASIEKTISKAKGQPR
EPQVCTLPPSRDELTKNQVSLSCAVKGFYPSDIAVEWESNGQPENNYKTT
PPVLDSDGSFFLVSKLTVDKSRWQQGNVFSCSVLHEALHSHYTQKSLSLS
PGK

SEQ ID NO: 136 is the amino acid sequence defining the 2-7 derived light chain of the 2-7/4-8(39/51) antibody 2-7VLCH1

QSALAQPASVSGSPGQSITISCTGTSSDVGAYNYVSWYQQHPGKAPKLMI
YDVSKRPSGVSNRFSGSKSGNTASLTISGLQAEDEADYYCSSYTTSSTVF
GGGTKLTVLSSASTKGPSVFPLAPSSKSTSGGTAALGCLVKDYFPEPVTV
SWNSGALTSGVHTFPAVLQSSGLYSLSSVVTVPSSSLGTQTYICNVNHKP
SNTKVDKKVEPKSC

SEQ ID NO: 137 is the amino acid sequence defining the 4-8(39/51) derived heavy chain of the 2-7/4-8(39/51) antibody 4-8(39/51) HC-Knob

QVQLVQSGAEVKKAGSSVKVSCKASGGTFSSHTITWVRLAPGQGLEWMGR
NIPILGIANYAQKFQGRVTITADKSTSTAYMELSSLRSEDTAVYYCASLQ
TVDTAIEKYYGMDVWGQGTTVTVSSASTKGPSVFPLAPSSKSTSGGTAAL
GCLVKDYFPEPVTVSWNSGALTSGVHTFPAVLQSSGLYSLSSVVTVPSSS
LGTQTYICNVNHKPSNTKVDKKVEPKSCDKTHTCPPCPAPEFEGGPSVFL
FPPKPKDTLMISRTPEVTCVVVDVSHEDPEVKFNWYVDGVEVHNAKTKPR
EEQYNSTYRVVSVLTVLHQDWLNGKEYKCKVSNKALPASIEKTISKAKGQ
PREPQVYTLPPCRDELTKNQVSLWCLVKGFYPSDIAVEWESNGQPENNYK
TTPPVLDSDGSFFLYSKLTVDKSRWQQGNVFSCSVLHEALHSHYTQKSLS
LSPGK

SEQ ID NO: 138 is the amino acid sequence defining the 4-8(39/51) derived light chain of the 2-7/4-8(39/51) antibody 4-8LC

SYELTQPPSVSVSPGQTASITCSGDKLGDKYACWYQQKPGQSPVLVIYQD
NKRPSGIPERFSGSNSGNTATLTISGTQAMDEADYYCQAWDSSTAVFGGG
TKLTVLGQPKAAPSVTLFPPSSEELQANKATLVCLISDFYPGAVTVAWKA
DSSPVKAGVETTTPSKQSNNKYAASSYLSLTPEQWKSHRSYSCQVTHEGS
TVEKTVAPTECS

The 1-57/1-87 bispecific antibody sequence can comprise:

SEQ ID NO: 139 is the amino acid sequence defining the 1-57 derived heavy chain of the 1-57/1-87 antibody 1-57HC-Hole-Cross

EVHLVESGGGLVQPGGSLRLSCAASGFTFSDHYMDWVRQAPGKGLEWVGR
TRNKANSYTTEYAASVKGRFTISRDDAKNSLYLQMNSLKTEDTAVYYCAR
VHRWAYCINGVCFGAYSDYWGQGTLVTVSSASTAAPSVFIFPPSDEQLKS
GTASVVCLLNNFYPREAKVQWKVDNALQSGNSQESVTEQDSKDSTYSLSS
TLTLSKADYEKHKVYACEVTHQGLSSPVTKSFNRGECDKTHTCPPCPAPE
FEGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPASIE
KTISKAKGQPREPQVCTLPPSRDELTKNQVSLSCAVKGFYPSDIAVEWES
NGQPENNYKTTPPVLDSDGSFFLVSKLTVDKSRWQQGNVFSCSVLHEALH
SHYTQKSLSLSPGK

SEQ ID NO: 140 amino acid sequence defining the 1-57 derived light chain of the 1-57/1-87 antibody 1-57VLCH1

EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPSTFG
QGTKLEIKSSASTKGPSVFPLAPSSKSTSGGTAALGCLVKDYFPEPVTVS
WNSGALTSGVHTFPAVLQSSGLYSLSSVVTVPSSSLGTQTYICNVNHKPS
NTKVDKKVEPKSC

SEQ ID NO: 141 is the amino acid sequence defining the 1-87 derived heavy chain of the 1-57/1-87 antibody 1-87HC-Knob

QVQLVQSGAEVKKPGASVKVSCKVSGYTLIELSMHWVRQAPGKGLEWMGG
FDPEDAETIYAQKFQGRVTMTEDTSTDTAYMELSSLRSEDTAVYYCATGI
AVIGPPPSTYYYYGMDVWGQGTTVTVSSASTKGPSVFPLAPSSKSTSGGT
AALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPEFEGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPASIEKTISKA
KGQPREPQVYTLPPCRDELTKNQVSLWCLVKGFYPSDIAVEWESNGQPEN
NYKTTPPVLDSDGSFFLYSKLTVDKSRWQQGNVFSCSVLHEALHSHYTQK
SLSLSPGK

SEQ ID NO: 142 is the amino acid sequence defining the 1-87 derived light chain of the 1-57/1-87 antibody 1-87LC

QSALTQPASVSGSPGQSITISCTGTSSDVGGYNYVSWYQQHPGKAPKLMI
YDVSKRPSGVSNRFSGSKSGNTASLTISGLQAEDEADYYCSSYTSSSTYV
FGTGTKVTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECS

The 1-57/4-8(39/51) bispecific antibody can comprise:

SEQ ID NO: 143 is the amino acid sequence defining the 1-57 derived heavy chain of the 1-57/4-8(39/51) antibody 1-57HC-Hole-Cross

EVHLVESGGGLVQPGGSLRLSCAASGFTFSDHYMDWVRQAPGKGLEWVGR
TRNKANSYTTEYAASVKGRFTISRDDAKNSLYLQMNSLKTEDTAVYYCAR
VHRWAYCINGVCFGAYSDYWGQGTLVTVSSASTAAPSVFIFPPSDEQLKS
GTASVVCLLNNFYPREAKVQWKVDNALQSGNSQESVTEQDSKDSTYSLSS
TLTLSKADYEKHKVYACEVTHQGLSSPVTKSFNRGECDKTHTCPPCPAPE
FEGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPASIE
KTISKAKGQPREPQVCTLPPSRDELTKNQVSLSCAVKGFYPSDIAVEWES
NGQPENNYKTTPPVLDSDGSFFLVSKLTVDKSRWQQGNVFSCSVLHEALH
SHYTQKSLSLSPGK

SEQ ID NO: 144 is the amino acid sequence defining the 1-57 derived light chain of the 1-57/4-8(39/51) antibody 1-57VLCH1

EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPSTFG
QGTKLEIKSSASTKGPSVFPLAPSSKSTSGGTAALGCLVKDYFPEPVTVS
WNSGALTSGVHTFPAVLQSSGLYSLSSVVTVPSSSLGTQTYICNVNHKPS
NTKVDKKVEPKSC

SEQ ID NO: 145 is the amino acid sequence defining the 4-8(39/51) derived heavy chain of the 1-57/4-8(39/51) antibody 4-8(39/51) HC-Knob

QVQLVQSGAEVKKAGSSVKVSCKASGGTFSSHTITWVRLAPGQGLEWMG
RNIPILGIANYAQKFQGRVTITADKSTSTAYMELSSLRSEDTAVYYCAS
LQTVDTAIEKYYGMDVWGQGTTVTVSSASTKGPSVFPLAPSSKSTSGGT
AALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEFEGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPASIEKT
ISKAKGQPREPQVYTLPPCRDELTKNQVSLWCLVKGFYPSDIAVEWESN
GQPENNYKTTPPVLDSDGSFFLYSKLTVDKSRWQQGNVFSCSVLHEALH
SHYTQKSLSLSPGK

SEQ ID NO: 146 is the amino acid sequence defining the 4-8(39/51) derived light chain of the 1-57/4-8(39/51) antibody 4-8(39/51)LC

SYELTQPPSVSVSPGQTASITCSGDKLGDKYACWYQQKPGQSPVLVIYQD
NKRPSGIPERFSGSNSGNTATLTISGTQAMDEADYYCQAWDSSTAVFGGG
TKLTVLGQPKAAPSVTLFPPSSEELQANKATLVCLISDFYPGAVTVAWKA
DSSPVKAGVETTTPSKQSNNKYAASSYLSLTPEQWKSHRSYSCQVTHEGS
TVEKTVAPTECS

The 2-7/2-51 bispecific antibody:

SEQ ID NO: 147 is the amino acid sequence defining the 2-7 derived heavy chain of the 2-7/2-51 antibody 2-7HC-Hole-Cross

QITLKESGPTLVKPTQTLTLTCTFSGFSLSTSGVGVGWIRQPPGKALEWL
ALIYWDDDKRYSPSLKSRLTITKDTSKNQVVLTMTNMDPVDTATYYCAHH
KIERIFDYWGQGTLVTVSSASTAAPSVFIFPPSDEQLKSGTASVVCLLNN
FYPREAKVQWKVDNALQSGNSQESVTEQDSKDSTYSLSSTLTLSKADYEK
HKVYACEVTHQGLSSPVTKSFNRGECDKTHTCPPCPAPEFEGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPASIEKTISKAKGQPR
EPQVCTLPPSRDELTKNQVSLSCAVKGFYPSDIAVEWESNGQPENNYKTT
PPVLDSDGSFFLVSKLTVDKSRWQQGNVFSCSVLHEALHSHYTQKSLSLS
PGK

SEQ ID NO: 148 is the amino acid sequence defining the 2-7 derived light chain of the 2-7/2-51 antibody 2-7VLCH1

QSALAQPASVSGSPGQSITISCTGTSSDVGAYNYVSWYQQHPGKAPKLMI
YDVSKRPSGVSNRFSGSKSGNTASLTISGLQAEDEADYYCSSYTTSSTVF
GGGTKLTVLSSASTKGPSVFPLAPSSKSTSGGTAALGCLVKDYFPEPVTV
SWNSGALTSGVHTFPAVLQSSGLYSLSSVVTVPSSSLGTQTYICNVNHKP
SNTKVDKKVEPKSC

SEQ ID NO: 149 is the amino acid sequence defining the 2-51 derived heavy chain of the 2-7/2-51 antibody 2-51 HC-Knob

QVQLVQSGAEVKKPGASVKVSCKVSGYTLIELSMHWVRQAPGKGLEWMGG
FDPEDVETIYAQQFQGRVTMTEDTSTDTAYMELSSLRSEDTAVYYCATGW
AYKSTWYFGYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPEFEGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPASIEKTISKAKGQPREP
QVYTLPPCRDELTKNQVSLWCLVKGFYPSDIAVEWESNGQPENNYKTTPP
VLDSDGSFFLYSKLTVDKSRWQQGNVFSCSVLHEALHSHYTQKSLSLSPG
K

SEQ ID NO: 150 is the amino acid sequence defining the 2-51 derived light chain of the 2-7/2-51 antibody 2-51LC

QSALTQPPSASGSPGQSVTISCTGTSSDVGGYNYVSWYQQHPGKAPKLMI
YEVSKRPSGVPDRFSGSKSGNTASLTVSGLQAEDEADYYCSSYAGSRMGF
GGGTKLTVLGQPKAAPSVTLFPPSSEELQANKATLVCLISDFYPGAVTVA
WKADSSPVKAGVETTTPSKQSNNKYAASSYLSLTPEQWKSHRSYSCQVTH
EGSTVEKTVAPTECS

The 2-15/4-8(39/51) bispecific antibody sequence can comprise:

SEQ ID NO: 151 is the amino acid sequence defining the 2-15 derived heavy chain of the 2-15/4-8(39/51) antibody 2-15HC-Hole-Cross

QVQLVQSGAEVKKPGASVRVSCKASGYTFTGYYMHWVRQAPGQGLEWMGW
INPISDGTNYAQKFQGWVTMTRDTSISTVYMELSRLRSDDTAVYYCARGG
SRCSGGNCYGWAYDAFDIWGQGTMITVSSASTAAPSVFIFPPSDEQLKSG
TASVVCLLNNFYPREAKVQWKVDNALQSGNSQESVTEQDSKDSTYSLSST
LTLSKADYEKHKVYACEVTHQGLSSPVTKSFNRGECDKTHTCPPCPAPEF
EGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPASIEK
TISKAKGQPREPQVCTLPPSRDELTKNQVSLSCAVKGFYPSDIAVEWESN
GQPENNYKTTPPVLDSDGSFFLVSKLTVDKSRWQQGNVFSCSVLHEALHS
HYTQKSLSLSPGK

SEQ ID NO: 152 is the amino acid sequence defining the 2-15 derived light chain of the 2-15/4-8(39/51) antibody 2-15VLCH1

QSALTQPASVSGSPGQSITISCTGTSSDVGGYNFVSWYQQHPGKAPKLMI
YDVSKRPSGVSNRFSGSKSGNTASLTISGLQAEDEADCYCSSYTSSSTFV
FGTGTKVTVLSSASTKGPSVFPLAPSSKSTSGGTAALGCLVKDYFPEPVT
VSWNSGALTSGVHTFPAVLQSSGLYSLSSVVTVPSSSLGTQTYICNVNHK
PSNTKVDKKVEPKSC

SEQ ID NO: 153 is the amino acid sequence defining the 4-8(39/51) derived heavy chain of the 2-15/4-8(39/51) antibody 4-8(39/51) HC-Knob

QVQLVQSGAEVKKAGSSVKVSCKASGGTFSSHTITWVRLAPGQGLEWMGR
NIPILGIANYAQKFQGRVTITADKSTSTAYMELSSLRSEDTAVYYCASLQ
TVDTAIEKYYGMDVWGQGTTVTVSSASTKGPSVFPLAPSSKSTSGGTAAL
GCLVKDYFPEPVTVSWNSGALTSGVHTFPAVLQSSGLYSLSSVVTVPSSS
LGTQTYICNVNHKPSNTKVDKKVEPKSCDKTHTCPPCPAPEFEGGPSVFL
FPPKPKDTLMISRTPEVTCVVVDVSHEDPEVKFNWYVDGVEVHNAKTKPR
EEQYNSTYRVVSVLTVLHQDWLNGKEYKCKVSNKALPASIEKTISKAKGQ
PREPQVYTLPPCRDELTKNQVSLWCLVKGFYPSDIAVEWESNGQPENNYK
TTPPVLDSDGSFFLYSKLTVDKSRWQQGNVFSCSVLHEALHSHYTQKSLS
LSPGK

SEQ ID NO: 154 is the amino acid sequence defining the 4-8(39/51) derived light chain of the 2-15/4-8(39/51) antibody 4-8(39/51)LC

SYELTQPPSVSVSPGQTASITCSGDKLGDKYACWYQQKPGQSPVLVIYQD
NKRPSGIPERFSGSNSGNTATLTISGTQAMDEADYYCQAWDSSTAVFGGG
TKLTVLGQPKAAPSVTLFPPSSEELQANKATLVCLISDFYPGAVTVAWKA
DSSPVKAGVETTTPSKQSNNKYAASSYLSLTPEQWKSHRSYSCQVTHEGS
TVEKTVAPTECS

The 1-57/4-18 bispecific antibody sequence can comprise:

SEQ ID NO: 155 is the amino acid sequence defining the 1-57 derived heavy chain of the 1-57/4-18 antibody 1-57HC-Hole-Cross

EVHLVESGGGLVQPGGSLRLSCAASGFTFSDHYMDWVRQAPGKGLEWVGR
TRNKANSYTTEYAASVKGRFTISRDDAKNSLYLQMNSLKTEDTAVYYCAR
VHRWAYCINGVCFGAYSDYWGQGTLVTVSSASTAAPSVFIFPPSDEQLKS
GTASVVCLLNNFYPREAKVQWKVDNALQSGNSQESVTEQDSKDSTYSLSS
TLTLSKADYEKHKVYACEVTHQGLSSPVTKSFNRGECDKTHTCPPCPAPE
FEGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPASIE
KTISKAKGQPREPQVCTLPPSRDELTKNQVSLSCAVKGFYPSDIAVEWES
NGQPENNYKTTPPVLDSDGSFFLVSKLTVDKSRWQQGNVFSCSVLHEALH
SHYTQKSLSLSPGK

SEQ ID NO: 156 is the amino acid sequence defining the 1-57 derived light chain of the 1-57/4-18 antibody 1-57VLCH1

EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPSTFG
QGTKLEIKSSASTKGPSVFPLAPSSKSTSGGTAALGCLVKDYFPEPVTVS
WNSGALTSGVHTFPAVLQSSGLYSLSSVVTVPSSSLGTQTYICNVNHKPS
NTKVDKKVEPKSC

SEQ ID NO: 157 is the amino acid sequence defining the 4-18 derived heavy chain of the 1-57/4-18 antibody 4-18HC-Knob

QVQLVESGGGVVQPGRSLRLSCAASGFTFSSYGMHWVRQAPGKGLEWVAV
ISYDGSNKHYADSVKGRFTISRDNSKNTLYLQMNSLRAEDTAVYYCAKDS
GYNYGYSWFDPWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPEFEGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPASIEKTISKAKGQPRE
PQVYTLPPCRDELTKNQVSLWCLVKGFYPSDIAVEWESNGQPENNYKTTP
PVLDSDGSFFLYSKLTVDKSRWQQGNVFSCSVLHEALHSHYTQKSLSLSP
GK

SEQ ID NO: 158 is the amino acid sequence defining the 4-18 derived light chain of the 1-57/4-18 antibody 4-18LC

SYELTQPPSVSVSPGQTARITCSADALAKQYAYWYQQKPGQAPVLVIYKD
SERPSGIPERFSGSSSGTTVTLTISGVQAEDEADYYCQSTDNSGTYPNWV
FGGGTKLTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECS

The 2-7/1-68 bispecific antibody can comprise:

SEQ ID NO: 159 is the amino acid sequence defining the 2-7 derived heavy chain of the 2-7/1-68 antibody 2-7HC-Hole-Cross

QITLKESGPTLVKPTQTLTLTCTFSGFSLSTSGVGVGWIRQPPGKALEWL
ALIYWDDDKRYSPSLKSRLTITKDTSKNQVVLTMTNMDPVDTATYYCAHH
KIERIFDYWGQGTLVTVSSASTAAPSVFIFPPSDEQLKSGTASVVCLLNN
FYPREAKVQWKVDNALQSGNSQESVTEQDSKDSTYSLSSTLTLSKADYEK
HKVYACEVTHQGLSSPVTKSFNRGECDKTHTCPPCPAPEFEGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPASIEKTISKAKGQPR
EPQVCTLPPSRDELTKNQVSLSCAVKGFYPSDIAVEWESNGQPENNYKTT
PPVLDSDGSFFLVSKLTVDKSRWQQGNVFSCSVLHEALHSHYTQKSLSLS
PGK

SEQ ID NO: 160 is the amino acid sequence defining the 2-7 derived light chain of the 2-7/1-68 antibody 2-7VLCH1

QSALAQPASVSGSPGQSITISCTGTSSDVGAYNYVSWYQQHPGKAPKLMI
YDVSKRPSGVSNRFSGSKSGNTASLTISGLQAEDEADYYCSSYTTSSTVF
GGGTKLTVLSSASTKGPSVFPLAPSSKSTSGGTAALGCLVKDYFPEPVTV
SWNSGALTSGVHTFPAVLQSSGLYSLSSVVTVPSSSLGTQTYICNVNHKP
SNTKVDKKVEPKSC

SEQ ID NO: 161 is the amino acid sequence defining the 1-68 derived heavy chain of the 2-7/1-68 antibody 1-68HC-Knob

QVQLVQSGAEVKKPGASVKVSCKVSGYTLIELSMHWVRQAPGKGLEWMGG
FDPEDAETIYAQKFQGRVTMTEDTSTDTAYMELSSLRSEDTAVYYCATGW
AVAGSSDVWYYYYGMDVWGQGTTVTVSSASTKGPSVFPLAPSSKSTSGGT
AALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPEFEGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPASIEKTISKA
KGQPREPQVYTLPPCRDELTKNQVSLWCLVKGFYPSDIAVEWESNGQPEN
NYKTTPPVLDSDGSFFLYSKLTVDKSRWQQGNVFSCSVLHEALHSHYTQK
SLSLSPGK

SEQ ID NO: 162 is the amino acid sequence defining the 1-68 derived light chain of the 2-7/1-68 antibody 1-68LC

QSALTQPPSVSGSPGQSVTISCTGTSSDVGSYNRVSWYQQPPGTAPKLMI
YEVSNRPSGVPDRFSGSKSGNTASLTISGLQAEDEADYYCSSYTSSSTYV
FGTGTKVTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECS

The 4-20/4-18 bispecific antibody sequence can comprise:

SEQ ID NO: 163 is the amino acid sequence defining the 4-20 derived heavy chain of the 4-20/4-18 antibody 4-20HC-Hole-Cross

QVQLLQSGAEVKKPGASVKVSCKASGYSFTSYYMHWVRQAPGQGLEWMGI
INPSGGGTTYAQKFQGRVTMTRDTSTSTVYMELSSLRSEDTAVYYCARPG
GGSYQEFDYWGQGTLVTVSSASTAAPSVFIFPPSDEQLKSGTASVVCLLN
NFYPREAKVQWKVDNALQSGNSQESVTEQDSKDSTYSLSSTLTLSKADYE
KHKVYACEVTHQGLSSPVTKSFNRGECDKTHTCPPCPAPEFEGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPASIEKTISKAKGQP
REPQVCTLPPSRDELTKNQVSLSCAVKGFYPSDIAVEWESNGQPENNYKT
TPPVLDSDGSFFLVSKLTVDKSRWQQGNVFSCSVLHEALHSHYTQKSLSL
SPGK

SEQ ID NO: 164 is the amino acid sequence defining the 4-20 derived light chain of the 4-20/4-18 antibody 4-20VLCH1

DIQMTQSPSSLSASVGDRVTITCRASQSISSYLNWYQQKPGKAPKLLISA
ASSLQSGVPSRFSGSGSGTDFTLTISSLQPEDFATYYCQQSYNTLQVTFG
GGTKVEIKSSASTKGPSVFPLAPSSKSTSGGTAALGCLVKDYFPEPVTVS
WNSGALTSGVHTFPAVLQSSGLYSLSSVVTVPSSSLGTQTYICNVNHKPS
NTKVDKKVEPKSC

SEQ ID NO: 165 is the amino acid sequence defining the 4-18 derived heavy chain of the 4-20/4-18 antibody 4-18HC-Knob

QVQLVESGGGVVQPGRSLRLSCAASGFTFSSYGMHWVRQAPGKGLEWVAV
ISYDGSNKHYADSVKGRFTISRDNSKNTLYLQMNSLRAEDTAVYYCAKDS
GYNYGYSWFDPWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPEFEGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPASIEKTISKAKGQPRE
PQVYTLPPCRDELTKNQVSLWCLVKGFYPSDIAVEWESNGQPENNYKTTP
PVLDSDGSFFLYSKLTVDKSRWQQGNVFSCSVLHEALHSHYTQKSLSLSP
GK

SEQ ID NO: 166 is the amino acid sequence defining the 4-18 derived light chain of the 4-20/4-18 antibody 4-18LC

SYELTQPPSVSVSPGQTARITCSADALAKQYAYWYQQKPGQAPVLVIYKD
SERPSGIPERFSGSSSGTTVTLTISGVQAEDEADYYCQSTDNSGTYPNWV
FGGGTKLTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECS

The 2-15 mut/2-51 bispecific antibody sequence can comprise:

SEQ ID NO: 167 is the amino acid sequence defining the 2-15 mut derived heavy chain of the 2-15 mut/2-51 antibody 2-15HC-Hole-Cross

QVQLVQSGAEVKKPGASVRVSCKASGYTFTGYYMHWVRQAPGQGLEWMGW
INPISDGTNYAQKFQGWVTMTRDTSISTVYMELSRLRSDDTAVYYCARGG
SRCSGGNCYGWAYDAFDIWGQGTMITVSSASTAAPSVFIFPPSDEQLKSG
TASVVCLLNNFYPREAKVQWKVDNALQSGNSQESVTEQDSKDSTYSLSST
LTLSKADYEKHKVYACEVTHQGLSSPVTKSFNRGECDKTHTCPPCPAPEF
EGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPASIEK
TISKAKGQPREPQVCTLPPSRDELTKNQVSLSCAVKGFYPSDIAVEWESN
GQPENNYKTTPPVLDSDGSFFLVSKLTVDKSRWQQGNVFSCSVLHEALHS
HYTQKSLSLSPGK

SEQ ID NO: 168 is the amino acid sequence defining the 2-15 mut derived light chain of the 2-15 mut/2-51 antibody 2-15 mutVLCH1

QSALTQPASVSGSPGQSITISCTGTSSDVGGYNFVSWYQQHPGKAPKLMI
YDVSKRPSGVSNRFSGSKSGNTASLTISGLQAEDEADSYCSSYTSSSTFV
FGTGTKVTVLSSASTKGPSVFPLAPSSKSTSGGTAALGCLVKDYFPEPVT
VSWNSGALTSGVHTFPAVLQSSGLYSLSSVVTVPSSSLGTQTYICNVNHK
PSNTKVDKKVEPKSC

SEQ ID NO: 169 is the amino acid sequence defining the 2-51 derived heavy chain of the 2-15 mut/2-51 antibody 2-51 HC-Knob

QVQLVQSGAEVKKPGASVKVSCKVSGYTLIELSMHWVRQAPGKGLEWMGG
FDPEDVETIYAQQFQGRVTMTEDTSTDTAYMELSSLRSEDTAVYYCATGW
AYKSTWYFGYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPEFEGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPASIEKTISKAKGQPREP
QVYTLPPCRDELTKNQVSLWCLVKGFYPSDIAVEWESNGQPENNYKTTPP
VLDSDGSFFLYSKLTVDKSRWQQGNVFSCSVLHEALHSHYTQKSLSLSPG
K

SEQ ID NO: 170 is the amino acid sequence defining the 2-51 derived light chain of the 2-15 mut/2-51 antibody 2-51LC

QSALTQPPSASGSPGQSVTISCTGTSSDVGGYNYVSWYQQHPGKAPKLMI
YEVSKRPSGVPDRFSGSKSGNTASLTVSGLQAEDEADYYCSSYAGSRMGF
GGGTKLTVLGQPKAAPSVTLFPPSSEELQANKATLVCLISDFYPGAVTVA
WKADSSPVKAGVETTTPSKQSNNKYAASSYLSLTPEQWKSHRSYSCQVTH
EGSTVEKTVAPTECS

The 4-20/4-8(39/51) bispecific antibody sequence can comprise:

SEQ ID NO: 171 is the amino acid sequence defining the 4-20 derived heavy chain of the 4-20/4-8(39/51) antibody 4-20HC-Hole-Cross

QVQLLQSGAEVKKPGASVKVSCKASGYSFTSYYMHWVRQAPGQGLEWMGI
INPSGGGTTYAQKFQGRVTMTRDTSTSTVYMELSSLRSEDTAVYYCARPG
GGSYQEFDYWGQGTLVTVSSASTAAPSVFIFPPSDEQLKSGTASVVCLLN
NFYPREAKVQWKVDNALQSGNSQESVTEQDSKDSTYSLSSTLTLSKADYE
KHKVYACEVTHQGLSSPVTKSFNRGECDKTHTCPPCPAPEFEGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPASIEKTISKAKGQP
REPQVCTLPPSRDELTKNQVSLSCAVKGFYPSDIAVEWESNGQPENNYKT
TPPVLDSDGSFFLVSKLTVDKSRWQQGNVFSCSVLHEALHSHYTQKSLSL
SPGK

SEQ ID NO: 172 is the amino acid sequence defining the 4-20 derived light chain of the 4-20/4-8(39/51) antibody 4-20VLCH1

DIQMTQSPSSLSASVGDRVTITCRASQSISSYLNWYQQKPGKAPKLLISA
ASSLQSGVPSRFSGSGSGTDFTLTISSLQPEDFATYYCQQSYNTLQVTFG
GGTKVEIKSSASTKGPSVFPLAPSSKSTSGGTAALGCLVKDYFPEPVTVS
WNSGALTSGVHTFPAVLQSSGLYSLSSVVTVPSSSLGTQTYICNVNHKPS
NTKVDKKVEPKSC

SEQ ID NO: 173 is the amino acid sequence defining the 4-8(39/51) derived heavy chain of the 4-20/4-8(39/51) antibody 4-8(39/51) HC-Knob

QVQLVQSGAEVKKAGSSVKVSCKASGGTFSSHTITWVRLAPGQGLEW
MGRNIPILGIANYAQKFQGRVTITADKSTSTAYMELSSLRSEDTAVY
YCASLQTVDTAIEKYYGMDVWGQGTTVTVSSASTKGPSVFPLAPSSK
STSGGTAALGCLVKDYFPEPVTVSWNSGALTSGVHTFPAVLQSSGLY
SLSSVVTVPSSSLGTQTYICNVNHKPSNTKVDKKVEPKSCDKTHTCP
PCPAPEFEGGPSVFLFPPKPKDTLMISRTPEVTCVVVDVSHEDPEVK
FNWYVDGVEVHNAKTKPREEQYNSTYRVVSVLTVLHQDWLNGKEYKC
KVSNKALPASIEKTISKAKGQPREPQVYTLPPCRDELTKNQVSLWCL
VKGFYPSDIAVEWESNGQPENNYKTTPPVLDSDGSFFLYSKLTVDKS
RWQQGNVFSCSVLHEALHSHYTQKSLSLSPGK

SEQ ID NO: 174 is the amino acid sequence defining the 4-8(39/51) derived light chain of the 4-20/4-8(39/51) antibody 4-8(39/51)LC

SYELTQPPSVSVSPGQTASITCSGDKLGDKYACWYQQKPGQSPVLVI
YQDNKRPSGIPERFSGSNSGNTATLTISGTQAMDEADYYCQAWDSST
AVFGGGTKLTVLGQPKAAPSVTLFPPSSEELQANKATLVCLISDFYP
GAVTVAWKADSSPVKAGVETTTPSKQSNNKYAASSYLSLTPEQWKSH
RSYSCQVTHEGSTVEKTVAPTECS

The 1-57/2-51 bispecific antibody sequence can comprise:

SEQ ID NO: 175 is the amino acid sequence defining the 1-57 derived heavy chain of the 1-57/2-51 antibody 1-57HC-Hole-Cross

EVHLVESGGGLVQPGGSLRLSCAASGFTFSDHYMDWVRQAPGKGLEW
VGRTRNKANSYTTEYAASVKGRFTISRDDAKNSLYLQMNSLKTEDTA
VYYCARVHRWAYCINGVCFGAYSDYWGQGTLVTVSSASTAAPSVFIF
PPSDEQLKSGTASVVCLLNNFYPREAKVQWKVDNALQSGNSQESVTE
QDSKDSTYSLSSTLTLSKADYEKHKVYACEVTHQGLSSPVTKSFNRG
ECDKTHTCPPCPAPEFEGGPSVFLFPPKPKDTLMISRTPEVTCVVVD
VSHEDPEVKFNWYVDGVEVHNAKTKPREEQYNSTYRVVSVLTVLHQD
WLNGKEYKCKVSNKALPASIEKTISKAKGQPREPQVCTLPPSRDELT
KNQVSLSCAVKGFYPSDIAVEWESNGQPENNYKTTPPVLDSDGSFFL
VSKLTVDKSRWQQGNVFSCSVLHEALHSHYTQKSLSLSPGK

SEQ ID NO: 176 is the amino acid sequence defining the 1-57 derived light chain of the 1-57/2-51 antibody 1-57VLCH1

EIVLTQSPGTLSLSPGERATLSCRASQSVSSSYLAWYQQKPGQAPRL
LIYGASSRATGIPDRFSGSGSGTDFTLTISRLEPEDFAVYYCQQYGS
SPSTFGQGTKLEIKSSASTKGPSVFPLAPSSKSTSGGTAALGCLVKD
YFPEPVTVSWNSGALTSGVHTFPAVLQSSGLYSLSSVVTVPSSSLGT
QTYICNVNHKPSNTKVDKKVEPKSC

SEQ ID NO: 177 is the amino acid sequence defining the 2-51 derived heavy chain of the 1-57/2-51 antibody 2-51 HC-Knob

QVQLVQSGAEVKKPGASVKVSCKVSGYTLIELSMHWVRQAPGKGLEW
MGGFDPEDVETIYAQQFQGRVTMTEDTSTDTAYMELSSLRSEDTAVY
YCATGWAYKSTWYFGYWGQGTLVTVSSASTKGPSVFPLAPSSKSTSG
GTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPA
PEFEGGPSVFLFPPKPKDTLMISRTPEVTCVVVDVSHEDPEVKFNWY
VDGVEVHNAKTKPREEQYNSTYRVVSVLTVLHQDWLNGKEYKCKVSN
KALPASIEKTISKAKGQPREPQVYTLPPCRDELTKNQVSLWCLVKGF
YPSDIAVEWESNGQPENNYKTTPPVLDSDGSFFLYSKLTVDKSRWQQ
GNVFSCSVLHEALHSHYTQKSLSLSPGK

SEQ ID NO: 178 is the amino acid sequence defining the 2-51 derived light chain of the 1-57/2-51 antibody 2-51LC

QSALTQPPSASGSPGQSVTISCTGTSSDVGGYNYVSWYQQHPGKAPK
LMIYEVSKRPSGVPDRFSGSKSGNTASLTVSGLQAEDEADYYCSSYA
GSRMGFGGGTKLTVLGQPKAAPSVTLFPPSSEELQANKATLVCLISD
FYPGAVTVAWKADSSPVKAGVETTTPSKQSNNKYAASSYLSLTPEQW
KSHRSYSCQVTHEGSTVEKTVAPTECS

The 4-20/1-87 bispecific antibody sequence can comprise:

SEQ ID NO: 179 is the amino acid sequence defining the 4-20 derived heavy chain of the 4-20/1-87 antibody 4-20HC-Hole-Cross

QVQLLQSGAEVKKPGASVKVSCKASGYSFTSYYMHWVRQAPGQGLEW
MGIINPSGGGTTYAQKFQGRVTMTRDTSTSTVYMELSSLRSEDTAVY
YCARPGGGSYQEFDYWGQGTLVTVSSASTAAPSVFIFPPSDEQLKSG
TASVVCLLNNFYPREAKVQWKVDNALQSGNSQESVTEQDSKDSTYSL
SSTLTLSKADYEKHKVYACEVTHQGLSSPVTKSFNRGECDKTHTCPP
CPAPEFEGGPSVFLFPPKPKDTLMISRTPEVTCVVVDVSHEDPEVKF
NWYVDGVEVHNAKTKPREEQYNSTYRVVSVLTVLHQDWLNGKEYKCK
VSNKALPASIEKTISKAKGQPREPQVCTLPPSRDELTKNQVSLSCAV
KGFYPSDIAVEWESNGQPENNYKTTPPVLDSDGSFFLVSKLTVDKSR
WQQGNVFSCSVLHEALHSHYTQKSLSLSPGK

SEQ ID NO: 180 is the amino acid sequence defining the 4-20 derived light chain of the 4-20/1-87 antibody 4-20VLCH1

DIQMTQSPSSLSASVGDRVTITCRASQSISSYLNWYQQKPGKAPKLL
ISAASSLQSGVPSRFSGSGSGTDFTLTISSLQPEDFATYYCQQSYNT
LQVTFGGGTKVEIKSSASTKGPSVFPLAPSSKSTSGGTAALGCLVKD
YFPEPVTVSWNSGALTSGVHTFPAVLQSSGLYSLSSVVTVPSSSLGT
QTYICNVNHKPSNTKVDKKVEPKSC

SEQ ID NO: 181 is the amino acid sequence defining the 1-87 derived heavy chain of the 4-20/1-87 antibody 1-87HC-Knob

QVQLVQSGAEVKKPGASVKVSCKVSGYTLIELSMHWVRQAPGKGLEW
MGGFDPEDAETIYAQKFQGRVTMTEDTSTDTAYMELSSLRSEDTAVY
YCATGIAVIGPPPSTYYYYGMDVWGQGTTVTVSSASTKGPSVFPLAP
SSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTH
TCPPCPAPEFEGGPSVFLFPPKPKDTLMISRTPEVTCVVVDVSHEDP
EVKFNWYVDGVEVHNAKTKPREEQYNSTYRVVSVLTVLHQDWLNGKE
YKCKVSNKALPASIEKTISKAKGQPREPQVYTLPPCRDELTKNQVSL
WCLVKGFYPSDIAVEWESNGQPENNYKTTPPVLDSDGSFFLYSKLTV
DKSRWQQGNVFSCSVLHEALHSHYTQKSLSLSPGK

SEQ ID NO: 182 is the amino acid sequence defining the 1-87 derived light chain of the 4-20/1-87 antibody 1-87LC

QSALTQPASVSGSPGQSITISCTGTSSDVGGYNYVSWYQQHPGKAPK
LMIYDVSKRPSGVSNRFSGSKSGNTASLTISGLQAEDEADYYCSSYT
SSSTYVFGTGTKVTVLGQPKAAPSVTLFPPSSEELQANKATLVCLIS
DFYPGAVTVAWKADSSPVKAGVETTTPSKQSNNKYAASSYLSLTPEQ
WKSHRSYSCQVTHEGSTVEKTVAPTECS

The 1-57/1-68 bispecific antibody sequence can comprise:

SEQ ID NO: 183 is the amino acid sequence defining the 1-57 derived heavy chain of the 1-57/1-68 antibody 1-57HC-Hole-Cross

EVHLVESGGGLVQPGGSLRLSCAASGFTFSDHYMDWVRQAPGKGLEW
VGRTRNKANSYTTEYAASVKGRFTISRDDAKNSLYLQMNSLKTEDTA
VYYCARVHRWAYCINGVCFGAYSDYWGQGTLVTVSSASTAAPSVFIF
PPSDEQLKSGTASVVCLLNNFYPREAKVQWKVDNALQSGNSQESVTE
QDSKDSTYSLSSTLTLSKADYEKHKVYACEVTHQGLSSPVTKSFNRG
ECDKTHTCPPCPAPEFEGGPSVFLFPPKPKDTLMISRTPEVTCVVVD
VSHEDPEVKFNWYVDGVEVHNAKTKPREEQYNSTYRVVSVLTVLHQD
WLNGKEYKCKVSNKALPASIEKTISKAKGQPREPQVCTLPPSRDELT
KNQVSLSCAVKGFYPSDIAVEWESNGQPENNYKTTPPVLDSDGSFFL
VSKLTVDKSRWQQGNVFSCSVLHEALHSHYTQKSLSLSPGK

SEQ ID NO: 184 is the amino acid sequence defining the 1-57 derived light chain of the 1-57/1-68 antibody 1-57VLCH1

EIVLTQSPGTLSLSPGERATLSCRASQSVSSSYLAWYQQKPGQAPRL
LIYGASSRATGIPDRFSGSGSGTDFTLTISRLEPEDFAVYYCQQYGS
SPSTFGQGTKLEIKSSASTKGPSVFPLAPSSKSTSGGTAALGCLVKD
YFPEPVTVSWNSGALTSGVHTFPAVLQSSGLYSLSSVVTVPSSSLGT
QTYICNVNHKPSNTKVDKKVEPKSC

SEQ ID NO: 185 is the amino acid sequence defining the 1-68 derived heavy chain of the 1-57/1-68 antibody 1-68HC-Knob

QVQLVQSGAEVKKPGASVKVSCKVSGYTLIELSMHWVRQAPGKGLEW
MGGFDPEDAETIYAQKFQGRVTMTEDTSTDTAYMELSSLRSEDTAVY
YCATGWAVAGSSDVWYYYYGMDVWGQGTTVTVSSASTKGPSVFPLAP
SSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTH
TCPPCPAPEFEGGPSVFLFPPKPKDTLMISRTPEVTCVVVDVSHEDP
EVKFNWYVDGVEVHNAKTKPREEQYNSTYRVVSVLTVLHQDWLNGKE
YKCKVSNKALPASIEKTISKAKGQPREPQVYTLPPCRDELTKNQVSL
WCLVKGFYPSDIAVEWESNGQPENNYKTTPPVLDSDGSFFLYSKLTV
DKSRWQQGNVFSCSVLHEALHSHYTQKSLSLSPGK

SEQ ID NO: 186 is the amino acid sequence defining the 1-68 derived light chain of the 1-57/1-68 antibody 1-68LC

QSALTQPPSVSGSPGQSVTISCTGTSSDVGSYNRVSWYQQPPGTAPK
LMIYEVSNRPSGVPDRFSGSKSGNTASLTISGLQAEDEADYYCSSYT
SSSTYVFGTGTKVTVLGQPKAAPSVTLFPPSSEELQANKATLVCLIS
DFYPGAVTVAWKADSSPVKAGVETTTPSKQSNNKYAASSYLSLTPEQ
WKSHRSYSCQVTHEGSTVEKTVAPTECS

Bispecific antibodies with LS mutations only

The 2-7/4-8(39/51/57) bispecific antibody sequence can comprise:

SEQ ID NO: 187 is the amino acid sequence defining the 2-7 derived heavy chain of the 2-7/4-8(39/51/57) antibody 2-7HC-Hole-Cross

QITLKESGPTLVKPTQTLTLTCTFSGFSLSTSGVGVGWIRQPPGKAL
EWLALIYWDDDKRYSPSLKSRLTITKDTSKNQVVLTMTNMDPVDTAT
YYCAHHKIERIFDYWGQGTLVTVSSASTAAPSVFIFPPSDEQLKSGT
ASVVCLLNNFYPREAKVQWKVDNALQSGNSQESVTEQDSKDSTYSLS
STLTLSKADYEKHKVYACEVTHQGLSSPVTKSFNRGECDKTHTCPPC
PAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKV
SNKALPAPIEKTISKAKGQPREPQVCTLPPSRDELTKNQVSLSCAVK
GFYPSDIAVEWESNGQPENNYKTTPPVLDSDGSFFLVSKLTVDKSRW
QQGNVFSCSVLHEALHSHYTQKSLSLSPGK

SEQ ID NO: 188 is the amino acid sequence defining the 2-7 derived light chain of the 2-7/4-8(39/51/57) antibody 2-7VLCH1

QSALAQPASVSGSPGQSITISCTGTSSDVGAYNYVSWYQQHPGKAPK
LMIYDVSKRPSGVSNRFSGSKSGNTASLTISGLQAEDEADYYCSSYT
TSSTVFGGGTKLTVLSSASTKGPSVFPLAPSSKSTSGGTAALGCLVK
DYFPEPVTVSWNSGALTSGVHTFPAVLQSSGLYSLSSVVTVPSSSLG
TQTYICNVNHKPSNTKVDKKVEPKSC

SEQ ID NO: 189 is the amino acid sequence defining the 4-8(39/51/57) derived heavy chain of the 2-7/4-8(39/51/57) antibody 4-8(39/51/57) HC-Knob

QVQLVQSGAEVKKAGSSVKVSCKASGGTFSSHTITWVRLAPGQ
GLEWMGRNIPILGTANYAQKFQGRVTITADKSTSTAYMELSSL
RSEDTAVYYCASLQTVDTAIEKYYGMDVWGQGTTVTVSSASTK
GPSVFPLAPSSKSTSGGTAALGCLVKDYFPEPVTVSWNSGALT
SGVHTFPAVLQSSGLYSLSSVVTVPSSSLGTQTYICNVNHKPS
NTKVDKKVEPKSCDKTHTCPPCPAPELLGGPSVFLFPPKPKDT
LMISRTPEVTCVVVDVSHEDPEVKFNWYVDGVEVHNAKTKPRE
EQYNSTYRVVSVLTVLHQDWLNGKEYKCKVSNKALPAPIEKTI
SKAKGQPREPQVYTLPPCRDELTKNQVSLWCLVKGFYPSDIAV
EWESNGQPENNYKTTPPVLDSDGSFFLYSKLTVDKSRWQQGNV
FSCSVLHEALHSHYTQKSLSLSPGK

SEQ ID NO: 190 is the amino acid sequence defining the 4-8(39/51/57) derived light chain of the 2-7/4-8(39/51/57) antibody 4-8LC

SYELTQPPSVSVSPGQTASITCSGDKLGDKYACWY
QQKPGQSPVLVIYQDNKRPSGIPERFSGSNSGNTA
TLTISGTQAMDEADYYCQAWDSSTAVFGGGTKLTV
LGQPKAAPSVTLFPPSSEELQANKA
TLVCLISDFYPGAVTVAWKADSSPVKAGVETTTPS
KQSNNKYAASSYLSLTPEQWKSHRSYSCQVTHEGS
TVEKTVAPTECS

The 2-17/2-36 bispecific antibody sequence can comprise:

SEQ ID NO: 191 is the amino acid sequence defining the 2-17 derived heavy chain of the 2-17/2-36 antibody 2-17HC-Hole-Cross

QVQLVQSGAEVKKPGSSVKVSCKASGGTFSSYAISW
VRQAPGQGLEWMGGNIPIFGTANYAQKFQGRVTIT
ADESTSTAYMELSSLRSEDTAVYYCARGVGYRGVI
PLNWFDPWGQGTVVTVSSASTAAPSVFIFPPSDEQ
LKSGTASVVCLLNNFYPREAKVQWKVDNALQSGNS
QESVTEQDSKDSTYSLSSTLTLSKADYEKHKVYAC
EVTHQGLSSPVTKSFNRGECDKTHTCPPCPAPELL
GGPSVFLFPPKPKDTLMISRTPEVTCVVVDVSHED
PEVKFNWYVDGVEVHNAKTKPREEQYNSTYRVVSV
LTVLHQDWLNGKEYKCKVSNKALPAPIEKTISKAK
GQPREPQVCTLPPSRDELTKNQVSLSCAVKGFYPS
DIAVEWESNGQPENNYKTTPPVLDSDGSFFLVSKL
TVDKSRWQQGNVFSCSVLHEALHSHYTQKSLSLSP
GK

SEQ ID NO: 192 is the amino acid sequence defining the 2-17 derived light chain of the 2-17/2-36 antibody 2-17VLCH1

EIVMTQSPATLSVSPGERATLSCRASQSVSSDLAWYQH
KPGQAPRLLIYGASTRATGIPVRFSGSGSGTEFTLTIS
SLQSEDFAVYYCQQYNNQPPFTFGGGTKVEIKSSASTK
GPSVFPLAPSSKSTSGGTAALGCLVKDYFPEPVTVSWN
SGALTSGVHTFPAVLQSSGLYSLSSVVTVPSSSLGTQT
YICNVNHKPSNTKVDKKVEPKSC

SEQ ID NO: 193 is the amino acid sequence defining the 2-36 derived heavy chain of the 2-17/2-36 antibody 2-36HC-Knob

QVQLQESGPGLVKPSETLSLTCTVSGGSVSSSNYYWSW
IRQPPGKGLEWIGYMYYSGSTKYNPSLKSRVTISVDTS
KNQFSLKLSSVTAADTAVYYCAREVYYYDRSGYYASDG
FDIWGQGTMVTVSSASTKGPSVFPLAPSSKSTSGGTAA
LGCLVKDYFPEPVTVSWNSGALTSGVHTFPAVLQSSGL
YSLSSVVTVPSSSLGTQTYICNVNHKPSNTKVDKKVEP
KSCDKTHTCPPCPAPELLGGPSVFLFPPKPKDTLMISR
TPEVTCVVVDVSHEDPEVKFNWYVDGVEVHNAKTKPRE
EQYNSTYRVVSVLTVLHQDWLNGKEYKCKVSNKALPAP
IEKTISKAKGQPREPQVYTLPPCRDELTKNQVSLWCLV
KGFYPSDIAVEWESNGQPENNYKTTPPVLDSDGSFFLY
SKLTVDKSRWQQGNVFSCSVLHEALHSHYTQKSLSLSP
GK

SEQ ID NO: 194 is the amino acid sequence defining the 2-36 derived light chain of the 2-17/2-36 antibody 2-36LC

EIVLTQSPGTLSLSPGERATLSCRASQSVSSSYLA
WYQQKPGQAPRLLIYGASSRATGIPDRFSGSGSGT
DFTLTISRLEPEDFAVYYCQQYGSSPQTFGQGTKV
EIKRTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC

The 2-36/1-68 bispecific antibody sequence:

SEQ ID NO: 195 is the amino acid sequence defining the 2-36 derived heavy chain of the 2-36/1-68 antibody 2-36HC-Hole-Cross

QVQLQESGPGLVKPSETLSLTCTVSGGSVSSSNYY
WSWIRQPPGKGLEWIGYMYYSGSTKYNPSLKSRVT
ISVDTSKNQFSLKLSSVTAADTAVYYCAREVYYYD
RSGYYASDGFDIWGQGTMVTVSSASTAAPSVFIFP
PSDEQLKSGTASVVCLLNNFYPREAKVQWKVDNAL
QSGNSQESVTEQDSKDSTYSLSSTLTLSKADYEKH
KVYACEVTHQGLSSPVTKSFNRGECDKTHTCPPCP
APELLGGPSVFLFPPKPKDTLMISRTPEVTCVVVD
VSHEDPEVKFNWYVDGVEVHNAKTKPREEQYNSTY
RVVSVLTVLHQDWLNGKEYKCKVSNKALPAPIEKT
ISKAKGQPREPQVCTLPPSRDELTKNQVSLSCAVK
GFYPSDIAVEWESNGQPENNYKTTPPVLDSDGSFF
LVSKLTVDKSRWQQGNVFSCSVLHEALHSHYTQKS
LSLSPGK

SEQ ID NO: 196 is the amino acid sequence defining the 2-36 derived light chain of the 2-36/1-68 antibody 2-36VLCH1

EIVLTQSPGTLSLSPGERATLSCRASQSVSSSYLA
WYQQKPGQAPRLLIYGASSRATGIPDRFSGSGSGT
DFTLTISRLEPEDFAVYYCQQYGSSPQTFGQGTKV
EIKSSASTKGPSVFPLAPSSKSTSGGTAALGCLVK
DYFPEPVTVSWNSGALTSGVHTFPAVLQSSGLYSL
SSVVTVPSSSLGTQTYICNVNHKPSNTKVDKKVEP
KSC

SEQ ID NO: 197 is the amino acid sequence defining the 1-68 derived heavy chain of the 2-36/1-68 antibody 1-68HC-Knob

QVQLVQSGAEVKKPGASVKVSCKVSGYTLIELSMH
WVRQAPGKGLEWMGGFDPEDAETTYAQKFQGRVTM
TEDTSTDTAYMELSSLRSEDTAVYYCATGWAVAGS
SDVWYYYYGMDVWGQGTTVTVSSASTKGPSVFPLA
PSSKSTSGGTAALGCLVKDYFPEPVTVSWNSGALT
SGVHTFPAVLQSSGLYSLSSVVTVPSSSLGTQTYI
CNVNHKPSNTKVDKKVEPKSCDKTHTCPPCPAPEL
LGGPSVFLFPPKPKDTLMISRTPEVTCVVVDVSHE
DPEVKFNWYVDGVEVHNAKTKPREEQYNSTYRVVS
VLTVLHQDWLNGKEYKCKVSNKALPAPIEKTISKA
KGQPREPQVYTLPPCRDELTKNQVSLWCLVKGFYP
SDIAVEWESNGQPENNYKTTPPVLDSDGSFFLYSK
LTVDKSRWQQGNVFSCSVLHEALHSHYTQKSLSLS
PGK

SEQ ID NO: 198 is the amino acid sequence defining the 1-68 derived light chain of the 2-36/1-68 antibody 1-68LC

QSALTQPPSVSGSPGQSVTISCTGTSSDVGSYNRV
SWYQQPPGTAPKLMIYEVSNRPSGVPDRFSGSKSG
NTASLTISGLQAEDEADYYCSSYTSSSTYVFGTGT
KVTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS

The 2-36/4-18 bispecific antibody sequence can comprise:

SEQ ID NO: 199 is the amino acid sequence defining the 2-36 derived heavy chain of the 2-36/4-18 antibody 2-36HC-Hole-Cross

QVQLQESGPGLVKPSETLSLTCTVSGGSVSSSNYY
WSWIRQPPGKGLEWIGYMYYSGSTKYNPSLKSRVT
ISVDTSKNQFSLKLSSVTAADTAVYYCAREVYYYD
RSGYYASDGFDIWGQGTMVTVSSASTAAPSVFIFP
PSDEQLKSGTASVVCLLNNFYPREAKVQWKVDNAL
QSGNSQESVTEQDSKDSTYSLSSTLTLSKADYEKH
KVYACEVTHQGLSSPVTKSFNRGECDKTHTCPPCP
APELLGGPSVFLFPPKPKDTLMISRTPEVTCVVVD
VSHEDPEVKFNWYVDGVEVHNAKTKPREEQYNSTY
RVVSVLTVLHQDWLNGKEYKCKVSNKALPAPIEKT
ISKAKGQPREPQVCTLPPSRDELTKNQVSLSCAVK
GFYPSDIAVEWESNGQPENNYKTTPPVLDSDGSFF
LVSKLTVDKSRWQQGNVFSCSVLHEALHSHYTQKS
LSLSPGK

SEQ ID NO: 200 is the amino acid sequence defining the 2-36 derived light chain of the 2-36/4-18 antibody 2-36VLCH1

EIVLTQSPGTLSLSPGERATLSCRASQSVSSSYLA
WYQQKPGQAPRLLIYGASSRATGIPDRFSGSGSGT
DFTLTISRLEPEDFAVYYCQQYGSSPQTFGQGTKV
EIKSSASTKGPSVFPLAPSSKSTSGGTAALGCLVK
DYFPEPVTVSWNSGALTSGVHTFPAVLQSSGLYSL
SSVVTVPSSSLGTQTYICNVNHKPSNTKVDKKVEP
KSC

SEQ ID NO: 201 is the amino acid sequence defining the 4-18 derived heavy chain of the 2-36/4-18 antibody 4-18HC-Knob

QVQLVESGGGVVQPGRSLRLSCAASGFTFSSYGMH
WVRQAPGKGLEWVAVISYDGSNKHYADSVKGRFTI
SRDNSKNTLYLQMNSLRAEDTAVYYCAKDSGYNYG
YSWFDPWGQGTLVTVSSASTKGPSVFPLAPSSKST
SGGTAALGCLVKDYFPEPVTVSWNSGALTSGVHTF
PAVLQSSGLYSLSSVVTVPSSSLGTQTYICNVNHK
PSNTKVDKKVEPKSCDKTHTCPPCPAPELLGGPSV
FLFPPKPKDTLMISRTPEVTCVVVDVSHEDPEVKF
NWYVDGVEVHNAKTKPREEQYNSTYRVVSVLTVLH
QDWLNGKEYKCKVSNKALPAPIEKTISKAKGQPRE
PQVYTLPPCRDELTKNQVSLWCLVKGFYPSDIAVE
WESNGQPENNYKTTPPVLDSDGSFFLYSKLTVDKS
RWQQGNVFSCSVLHEALHSHYTQKSLSLSPGK

SEQ ID NO: 202 is the amino acid sequence defining the 4-18 derived light chain of the 2-36/4-18 antibody 4-18LC

SYELTQPPSVSVSPGQTARITCSADALAKQYAYWY
QQKPGQAPVLVIYKDSERPSGIPERFSGSSSGTTV
TLTISGVQAEDEADYYCQSTDNSGTYPNWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS

The 2-30/1-68 bispecific antibody sequence can comprise:

SEQ ID NO: 203 is the amino acid sequence defining the 2-30 derived heavy chain of the 2-30/1-68 antibody 2-30HC-Hole-Cross

QVQLVESGGGVVQPGRSLRLSCAASGFTFSSYTMHWVRQAPGKGLEW
VAAISYDGNNKYYADSVKGRFTISRDNSKNTLYLQMNSLRAEDTAVY
YCARSILYGGGMDVWGQGTTVTVSSASTAAPSVFIFPPSDEQLKSGT
ASVVCLLNNFYPREAKVQWKVDNALQSGNSQESVTEQDSKDSTYSLS
STLTLSKADYEKHKVYACEVTHQGLSSPVTKSFNRGECDKTHTCPPC
PAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKV
SNKALPAPIEKTISKAKGQPREPQVCTLPPSRDELTKNQVSLSCAVK
GFYPSDIAVEWESNGQPENNYKTTPPVLDSDGSFFLVSKLTVDKSRW
QQGNVFSCSVLHEALHSHYTQKSLSLSPGK

SEQ ID NO: 204 is the amino acid sequence defining the 2-30 derived light chain of the 2-30/1-68 antibody 2-30VLCH1

DIQLTQSPSSLSASVGDRVTITCRASQGISSYLAWYQQKPGQAPKLL
IYAASTLQSGVPSRFSGSGSGTDFTLTISSLQPEDFATYYCQQLNSY
PLTFGGGTKVEIKSSASTKGPSVFPLAPSSKSTSGGTAALGCLVKDY
FPEPVTVSWNSGALTSGVHTFPAVLQSSGLYSLSSVVTVPSSSLGTQ
TYICNVNHKPSNTKVDKKVEPKSC

SEQ ID NO: 205 is the amino acid sequence defining the 1-68 derived heavy chain of the 2-30/1-68 antibody 1-68HC-Knob

QVQLVQSGAEVKKPGASVKVSCKVSGYTLIELSMHWVRQAPGKGLEW
MGGFDPEDAETIYAQKFQGRVTMTEDTSTDTAYMELSSLRSEDTAVY
YCATGWAVAGSSDVWYYYYGMDVWGQGTTVTVSSASTKGPSVFPLAP
SSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTH
TCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKE
YKCKVSNKALPAPIEKTISKAKGQPREPQVYTLPPCRDELTKNQVSL
WCLVKGFYPSDIAVEWESNGQPENNYKTTPPVLDSDGSFFLYSKLTV
DKSRWQQGNVFSCSVLHEALHSHYTQKSLSLSPGK

SEQ ID NO: 206 is the amino acid sequence defining the 1-68 derived light chain of the 2-30/1-68 antibody 1-68LC

QSALTQPPSVSGSPGQSVTISCTGTSSDVGSYNRVSWYQQPPGTAPK
LMIYEVSNRPSGVPDRFSGSKSGNTASLTISGLQAEDEADYYCSSYT
SSSTYVFGTGTKVTVLGQPKAAPSVTLFPPSSEELQANKATLVCLIS
DFYPGAVTVAWKADSSPVKAGVETTTPSKQSNNKYAASSYLSLTPEQ
WKSHRSYSCQVTHEGSTVEKTVAPTECS

The 2-30/4-18 bispecific antibody sequence can comprise:

SEQ ID NO: 207 is the amino acid sequence defining the 2-30 derived heavy chain of the 2-30/4-18 antibody 2-30HC-Hole-Cross

QVQLVESGGGVVQPGRSLRLSCAASGFTFSSYTMHWVRQAPGKGLEW
VAAISYDGNNKYYADSVKGRFTISRDNSKNTLYLQMNSLRAEDTAVY
YCARSILYGGGMDVWGQGTTVTVSSASTAAPSVFIFPPSDEQLKSGT
ASVVCLLNNFYPREAKVQWKVDNALQSGNSQESVTEQDSKDSTYSLS
STLTLSKADYEKHKVYACEVTHQGLSSPVTKSFNRGECDKTHTCPPC
PAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKV
SNKALPAPIEKTISKAKGQPREPQVCTLPPSRDELTKNQVSLSCAVK
GFYPSDIAVEWESNGQPENNYKTTPPVLDSDGSFFLVSKLTVDKSRW
QQGNVFSCSVLHEALHSHYTQKSLSLSPGK

SEQ ID NO: 208 is the amino acid sequence defining the 2-30 derived light chain of the 2-30/4-18 antibody 2-30VLCH1

DIQLTQSPSSLSASVGDRVTITCRASQGISSYLAWYQQKPGQAPKLL
IYAASTLQSGVPSRFSGSGSGTDFTLTISSLQPEDFATYYCQQLNSY
PLTFGGGTKVEIKSSASTKGPSVFPLAPSSKSTSGGTAALGCLVKDY
FPEPVTVSWNSGALTSGVHTFPAVLQSSGLYSLSSVVTVPSSSLGTQ
TYICNVNHKPSNTKVDKKVEPKSC

SEQ ID NO: 209 is the amino acid sequence defining the 4-18 derived heavy chain of the 2-30/4-18 antibody 4-18HC-Knob

QVQLVESGGGVVQPGRSLRLSCAASGFTFSSYGMHWVRQAPGKGLEW
VAVISYDGSNKHYADSVKGRFTISRDNSKNTLYLQMNSLRAEDTAVY
YCAKDSGYNYGYSWFDPWGQGTLVTVSSASTKGPSVFPLAPSSKSTS
GGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCP
APELLGGPSVFLFPPKPKDTLMISRTPEVTCVVVDVSHEDPEVKFNW
YVDGVEVHNAKTKPREEQYNSTYRVVSVLTVLHQDWLNGKEYKCKVS
NKALPAPIEKTISKAKGQPREPQVYTLPPCRDELTKNQVSLWCLVKG
FYPSDIAVEWESNGQPENNYKTTPPVLDSDGSFFLYSKLTVDKSRWQ
QGNVFSCSVLHEALHSHYTQKSLSLSPGK

SEQ ID NO: 210 is the amino acid sequence defining the 4-18 derived light chain of the 2-30/4-18 antibody 4-18LC

SYELTQPPSVSVSPGQTARITCSADALAKQYAYWYQQKPGQAPVLVI
YKDSERPSGIPERFSGSSSGTTVTLTISGVQAEDEADYYCQSTDNSG
TYPNWVFGGGTKLTVLGQPKAAPSVTLFPPSSEELQANKATLVCLIS
DFYPGAVTVAWKADSSPVKAGVETTTPSKQSNNKYAASSYLSLTPEQ
WKSHRSYSCQVTHEGSTVEKTVAPTECS

The 2-7/4-8(39/51) bispecific antibody sequence can comprise:

SEQ ID NO: 211 is the amino acid sequence defining the 2-7 derived heavy chain of the 2-7/4-8(39/51) antibody 2-7HC-Hole-Cross

QITLKESGPTLVKPTQTLTLTCTFSGFSLSTSGVGVGWIRQPPGKAL
EWLALIYWDDDKRYSPSLKSRLTITKDTSKNQVVLTMTNMDPVDTAT
YYCAHHKIERIFDYWGQGTLVTVSSASTAAPSVFIFPPSDEQLKSGT
ASVVCLLNNFYPREAKVQWKVDNALQSGNSQESVTEQDSKDSTYSLS
STLTLSKADYEKHKVYACEVTHQGLSSPVTKSFNRGECDKTHTCPPC
PAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKV
SNKALPAPIEKTISKAKGQPREPQVCTLPPSRDELTKNQVSLSCAVK
GFYPSDIAVEWESNGQPENNYKTTPPVLDSDGSFFLVSKLTVDKSRW
QQGNVFSCSVLHEALHSHYTQKSLSLSPGK

SEQ ID NO: 212 is the amino acid sequence defining the 2-7 derived light chain of the 2-7/4-8(39/51) antibody 2-7VLCH1

QSALAQPASVSGSPGQSITISCTGTSSDVGAYNYVSWYQQHPGKAPK
LMIYDVSKRPSGVSNRFSGSKSGNTASLTISGLQAEDEADYYCSSYT
TSSTVFGGGTKLTVLSSASTKGPSVFPLAPSSKSTSGGTAALGCLVK
DYFPEPVTVSWNSGALTSGVHTFPAVLQSSGLYSLSSVVTVPSSSLG
TQTYICNVNHKPSNTKVDKKVEPKSC

SEQ ID NO: 213 is the amino acid sequence defining the 4-8(39/51) derived heavy chain of the 2-7/4-8(39/51) antibody 4-8(39/51) HC-Knob

QVQLVQSGAEVKKAGSSVKVSCKASGGTFSSHTITWVRLAPGQGLEW
MGRNIPILGIANYAQKFQGRVTITADKSTSTAYMELSSLRSEDTAVY
YCASLQTVDTAIEKYYGMDVWGQGTTVTVSSASTKGPSVFPLAPSSK
STSGGTAALGCLVKDYFPEPVTVSWNSGALTSGVHTFPAVLQSSGLY
SLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKC
KVSNKALPAPIEKTISKAKGQPREPQVYTLPPCRDELTKNQVSLWCL
VKGFYPSDIAVEWESNGQPENNYKTTPPVLDSDGSFFLYSKLTVDKS
RWQQGNVFSCSVLHEALHSHYTQKSLSLSPGK

SEQ ID NO: 214 is the amino acid sequence defining the 4-8(39/51) derived light chain of the 2-7/4-8(39/51) antibody 4-8LC

SYELTQPPSVSVSPGQTASITCSGDKLGDKYACWYQQKPGQSPVLVI
YQDNKRPSGIPERFSGSNSGNTATLTISGTQAMDEADYYCQAWDSST
AVFGGGTKLTVLGQPKAAPSVTLFPPSSEELQANKATLVCLISDFYP
GAVTVAWKADSSPVKAGVETTTPSKQSNNKYAASSYLSLTPEQWKSH
RSYSCQVTHEGSTVEKTVAPTECS

The 1-57/1-87 bispecific antibody sequence can comprise:

SEQ ID NO: 215 is the amino acid sequence defining the 1-57 derived heavy chain of the 1-57/1-87 antibody 1-57HC-Hole-Cross

EVHLVESGGGLVQPGGSLRLSCAASGFTFSDHYMDWVRQAPGKGLEW
VGRTRNKANSYTTEYAASVKGRFTISRDDAKNSLYLQMNSLKTEDTA
VYYCARVHRWAYCINGVCFGAYSDYWGQGTLVTVSSASTAAPSVFIF
PPSDEQLKSGTASVVCLLNNFYPREAKVQWKVDNALQSGNSQESVTE
QDSKDSTYSLSSTLTLSKADYEKHKVYACEVTHQGLSSPVTKSFNRG
ECDKTHTCPPCPAPELLGGPSVFLFPPKPKDTLMISRTPEVTCVVVD
VSHEDPEVKFNWYVDGVEVHNAKTKPREEQYNSTYRVVSVLTVLHQD
WLNGKEYKCKVSNKALPAPIEKTISKAKGQPREPQVCTLPPSRDELT
KNQVSLSCAVKGFYPSDIAVEWESNGQPENNYKTTPPVLDSDGSFFL
VSKLTVDKSRWQQGNVFSCSVLHEALHSHYTQKSLSLSPGK

SEQ ID NO: 216 is the amino acid sequence defining the 1-57 derived light chain of the 1-57/1-87 antibody 1-57VLCH1

EIVLTQSPGTLSLSPGERATLSCRASQSVSSSYLAWYQQKPGQAPRL
LIYGASSRATGIPDRFSGSGSGTDFTLTISRLEPEDFAVYYCQQYGS
SPSTFGQGTKLEIKSSASTKGPSVFPLAPSSKSTSGGTAALGCLVKD
YFPEPVTVSWNSGALTSGVHTFPAVLQSSGLYSLSSVVTVPSSSLGT
QTYICNVNHKPSNTKVDKKVEPKSC

SEQ ID NO: 217 is the amino acid sequence defining the 1-87 derived heavy chain of the 1-57/1-87 antibody 1-87HC-Knob

QVQLVQSGAEVKKPGASVKVSCKVSGYTLIELSMHWVRQAPGKGLEW
MGGFDPEDAETIYAQKFQGRVTMTEDTSTDTAYMELSSLRSEDTAVY
YCATGIAVIGPPPSTYYYYGMDVWGQGTTVTVSSASTKGPSVFPLAP
SSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTH
TCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKE
YKCKVSNKALPAPIEKTISKAKGQPREPQVYTLPPCRDELTKNQVSL
WCLVKGFYPSDIAVEWESNGQPENNYKTTPPVLDSDGSFFLYSKLTV
DKSRWQQGNVFSCSVLHEALHSHYTQKSLSLSPGK

SEQ ID NO: 218 is the amino acid sequence defining the 1-87 derived light chain of the 1-57/1-87 antibody 1-87LC

QSALTQPASVSGSPGQSITISCTGTSSDVGGYNYVSWYQQHPGKAPKLM
IYDVSKRPSGVSNRFSGSKSGNTASLTISGLQAEDEADYYCSSYTSSST
YVFGTGTKVTVLGQPKAAPSVTLFPPSSEELQANKATLVCLISDFYPGA
VTVAWKADSSPVKAGVETTTPSKQSNNKYAASSYLSLTPEQWKSHRSYS
CQVTHEGSTVEKTVAPTECS

The 1-57/4-8(39/51) bispecific antibody sequence can comprise:

SEQ ID NO: 219 is the amino acid sequence defining the 1-57 derived heavy chain of the 1-57/4-8(39/51) antibody 1-57HC-Hole-Cross

EVHLVESGGGLVQPGGSLRLSCAASGFTFSDHYMDWVRQAPGKGLEWVG
RTRNKANSYTTEYAASVKGRFTISRDDAKNSLYLQMNSLKTEDTAVYYC
ARVHRWAYCINGVCFGAYSDYWGQGTLVTVSSASTAAPSVFIFPPSDEQ
LKSGTASVVCLLNNFYPREAKVQWKVDNALQSGNSQESVTEQDSKDSTY
SLSSTLTLSKADYEKHKVYACEVTHQGLSSPVTKSFNRGECDKTHTCPP
CPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNK
ALPAPIEKTISKAKGQPREPQVCTLPPSRDELTKNQVSLSCAVKGFYPS
DIAVEWESNGQPENNYKTTPPVLDSDGSFFLVSKLTVDKSRWQQGNVFS
CSVLHEALHSHYTQKSLSLSPGK

SEQ ID NO: 220 is the amino acid sequence defining the 1-57 derived light chain of the 1-57/4-8(39/51) antibody 1-57VLCH1

EIVLTQSPGTLSLSPGERATLSCRASQSVSSSYLAWYQQKPGQAPRLLI
YGASSRATGIPDRFSGSGSGTDFTLTISRLEPEDFAVYYCQQYGSSPST
FGQGTKLEIKSSASTKGPSVFPLAPSSKSTSGGTAALGCLVKDYFPEPV
TVSWNSGALTSGVHTFPAVLQSSGLYSLSSVVTVPSSSLGTQTYICNVN
HKPSNTKVDKKVEPKSC

SEQ ID NO: 221 is the amino acid sequence defining the 4-8(39/51) derived heavy chain of the 1-57/4-8(39/51) antibody 4-8(39/51) HC-Knob

QVQLVQSGAEVKKAGSSVKVSCKASGGTFSSHTITWVRLAPGQGLEWMG
RNIPILGIANYAQKFQGRVTITADKSTSTAYMELSSLRSEDTAVYYCAS
LQTVDTAIEKYYGMDVWGQGTTVTVSSASTKGPSVFPLAPSSKSTSGGT
AALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPCRDELTKNQVSLWCLVKGFYPSDIAVEWESN
GQPENNYKTTPPVLDSDGSFFLYSKLTVDKSRWQQGNVFSCSVLHEALH
SHYTQKSLSLSPGK

SEQ ID NO: 222 is the amino acid sequence defining the 4-8(39/51) derived light chain of the 1-57/4-8(39/51) antibody 4-8LC

SYELTQPPSVSVSPGQTASITCSGDKLGDKYACWYQQKPGQSPVLVIYQ
DNKRPSGIPERFSGSNSGNTATLTISGTQAMDEADYYCQAWDSSTAVFG
GGTKLTVLGQPKAAPSVTLFPPSSEELQANKATLVCLISDFYPGAVTVA
WKADSSPVKAGVETTTPSKQSNNKYAASSYLSLTPEQWKSHRSYSCQVT
HEGSTVEKTVAPTECS

The 2-7/2-51 bispecific antibody sequence can comprise:

SEQ ID NO: 223 is the amino acid sequence defining the 2-7 derived heavy chain of the 2-7/2-51 antibody 2-7HC-Hole-Cross

QITLKESGPTLVKPTQTLTLTCTFSGFSLSTSGVGVGWIRQPPGKALEW
LALIYWDDDKRYSPSLKSRLTITKDTSKNQVVLTMTNMDPVDTATYYCA
HHKIERIFDYWGQGTLVTVSSASTAAPSVFIFPPSDEQLKSGTASVVCL
LNNFYPREAKVQWKVDNALQSGNSQESVTEQDSKDSTYSLSSTLTLSKA
DYEKHKVYACEVTHQGLSSPVTKSFNRGECDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVCTLPPSRDELTKNQVSLSCAVKGFYPSDIAVEWESNGQ
PENNYKTTPPVLDSDGSFFLVSKLTVDKSRWQQGNVFSCSVLHEALHSH
YTQKSLSLSPGK

SEQ ID NO: 224 is the amino acid sequence defining the 2-7 derived light chain of the 2-7/2-51 antibody 2-7VLCH1

QSALAQPASVSGSPGQSITISCTGTSSDVGAYNYVSWYQQHPGKAPKLM
IYDVSKRPSGVSNRFSGSKSGNTASLTISGLQAEDEADYYCSSYTTSST
VFGGGTKLTVLSSASTKGPSVFPLAPSSKSTSGGTAALGCLVKDYFPEP
VTVSWNSGALTSGVHTFPAVLQSSGLYSLSSVVTVPSSSLGTQTYICNV
NHKPSNTKVDKKVEPKSC

SEQ ID NO: 225 is the amino acid sequence defining the 2-51 derived heavy chain of the 2-7/2-51 antibody 2-51 HC-Knob

QVQLVQSGAEVKKPGASVKVSCKVSGYTLIELSMHWVRQAPGKGLEWMG
GFDPEDVETIYAQQFQGRVTMTEDTSTDTAYMELSSLRSEDTAVYYCAT
GWAYKSTWYFGYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPCRDELTKNQVSLWCLVKGFYPSDIAVEWESNGQPE
NNYKTTPPVLDSDGSFFLYSKLTVDKSRWQQGNVFSCSVLHEALHSHYT
QKSLSLSPGK

SEQ ID NO: 226 is the amino acid sequence defining the 2-51 derived light chain of the 2-7/2-51 antibody 2-51LC

QSALTQPPSASGSPGQSVTISCTGTSSDVGGYNYVSWYQQHPGKAPKLM
IYEVSKRPSGVPDRFSGSKSGNTASLTVSGLQAEDEADYYCSSYAGSRM
GFGGGTKLTVLGQPKAAPSVTLFPPSSEELQANKATLVCLISDFYPGAV
TVAWKADSSPVKAGVETTTPSKQSNNKYAASSYLSLTPEQWKSHRSYSC
QVTHEGSTVEKTVAPTECS

The 2-15/4-8(39/51) bispecific antibody sequence can comprise:

SEQ ID NO: 227 is the amino acid sequence defining the 2-15 derived heavy chain of the 2-15/4-8(39/51) antibody 2-15HC-Hole-Cross

QVQLVQSGAEVKKPGASVRVSCKASGYTFTGYYMHWVRQAPGQGLEWMG
WINPISDGTNYAQKFQGWVTMTRDTSISTVYMELSRLRSDDTAVYYCAR
GGSRCSGGNCYGWAYDAFDIWGQGTMITVSSASTAAPSVFIFPPSDEQL
KSGTASVVCLLNNFYPREAKVQWKVDNALQSGNSQESVTEQDSKDSTYS
LSSTLTLSKADYEKHKVYACEVTHQGLSSPVTKSFNRGECDKTHTCPPC
PAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKA
LPAPIEKTISKAKGQPREPQVCTLPPSRDELTKNQVSLSCAVKGFYPSD
IAVEWESNGQPENNYKTTPPVLDSDGSFFLVSKLTVDKSRWQQGNVFSC
SVLHEALHSHYTQKSLSLSPGK

SEQ ID NO: 228 is the amino acid sequence defining the 2-15 derived light chain of the 2-15/4-8(39/51) antibody 2-15VLCH1

QSALTQPASVSGSPGQSITISCTGTSSDVGGYNFVSWYQQHPGKAPKLM
IYDVSKRPSGVSNRFSGSKSGNTASLTISGLQAEDEADCYCSSYTSSST
FVFGTGTKVTVLSSASTKGPSVFPLAPSSKSTSGGTAALGCLVKDYFPE
PVTVSWNSGALTSGVHTFPAVLQSSGLYSLSSVVTVPSSSLGTQTYICN
VNHKPSNTKVDKKVEPKSC

SEQ ID NO: 229 is the amino acid sequence defining the 4-8(39/51) derived heavy chain of the 2-15/4-8(39/51) antibody 4-8(39/51) HC-Knob

QVQLVQSGAEVKKAGSSVKVSCKASGGTFSSHTITWVRLAPGQGLEWMG
RNIPILGIANYAQKFQGRVTITADKSTSTAYMELSSLRSEDTAVYYCAS
LQTVDTAIEKYYGMDVWGQGTTVTVSSASTKGPSVFPLAPSSKSTSGGT
AALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPCRDELTKNQVSLWCLVKGFYPSDIAVEWESN
GQPENNYKTTPPVLDSDGSFFLYSKLTVDKSRWQQGNVFSCSVLHEALH
SHYTQKSLSLSPGK

SEQ ID NO: 230 is the amino acid sequence defining the 4-8(39/51) derived light chain of the 2-15/4-8(39/51) antibody 4-8LC

SYELTQPPSVSVSPGQTASITCSGDKLGDKYACWYQQKPGQSPVLVIYQ
DNKRPSGIPERFSGSNSGNTATLTISGTQAMDEADYYCQAWDSSTAVFG
GGTKLTVLGQPKAAPSVTLFPPSSEELQANKATLVCLISDFYPGAVTVA
WKADSSPVKAGVETTTPSKQSNNKYAASSYLSLTPEQWKSHRSYSCQVT
HEGSTVEKTVAPTECS

The 1-57/4-18 bispecific antibody sequence can comprise:

SEQ ID NO: 231 is the amino acid sequence defining the 1-57 derived heavy chain of the 1-57/4-18 antibody 1-57HC-Hole-Cross

EVHLVESGGGLVQPGGSLRLSCAASGFTFSDHYMDWVRQAPGKGLEWVG
RTRNKANSYTTEYAASVKGRFTISRDDAKNSLYLQMNSLKTEDTAVYYC
ARVHRWAYCINGVCFGAYSDYWGQGTLVTVSSASTAAPSVFIFPPSDEQ
LKSGTASVVCLLNNFYPREAKVQWKVDNALQSGNSQESVTEQDSKDSTY
SLSSTLTLSKADYEKHKVYACEVTHQGLSSPVTKSFNRGECDKTHTCPP
CPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNK
ALPAPIEKTISKAKGQPREPQVCTLPPSRDELTKNQVSLSCAVKGFYPS
DIAVEWESNGQPENNYKTTPPVLDSDGSFFLVSKLTVDKSRWQQGNVFS
CSVLHEALHSHYTQKSLSLSPGK

SEQ ID NO: 232 is the amino acid sequence defining the 1-57 derived light chain of the 1-57/4-18 antibody 1-57VLCH1

EIVLTQSPGTLSLSPGERATLSCRASQSVSSSYLAWYQQKPGQAPRLLI
YGASSRATGIPDRFSGSGSGTDFTLTISRLEPEDFAVYYCQQYGSSPST
FGQGTKLEIKSSASTKGPSVFPLAPSSKSTSGGTAALGCLVKDYFPEPV
TVSWNSGALTSGVHTFPAVLQSSGLYSLSSVVTVPSSSLGTQTYICNVN
HKPSNTKVDKKVEPKSC

SEQ ID NO: 233 is the amino acid sequence defining the 4-18 derived heavy chain of the 1-57/4-18 antibody 4-18HC-Knob

QVQLVESGGGVVQPGRSLRLSCAASGFTFSSYGMHWVRQAPGKGLEWVAV
ISYDGSNKHYADSVKGRFTISRDNSKNTLYLQMNSLRAEDTAVYYCAKDS
GYNYGYSWFDPWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPCRDELTKNQVSLWCLVKGFYPSDIAVEWESNGQPENNYKTTP
PVLDSDGSFFLYSKLTVDKSRWQQGNVFSCSVLHEALHSHYTQKSLSLSP
GK

SEQ ID NO: 234 is the amino acid sequence defining the 4-18 derived light chain of the 1-57/4-18 antibody 4-18LC

SYELTQPPSVSVSPGQTARITCSADALAKQYAYWYQQKPGQAPVLVIYKD
SERPSGIPERFSGSSSGTTVTLTISGVQAEDEADYYCQSTDNSGTYPNWV
FGGGTKLTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECS

The 2-7/1-68 bispecific antibody sequence can comprise:

SEQ ID NO: 235 is the amino acid sequence defining the 2-7 derived heavy chain of the 2-7/1-68 antibody 2-7HC-Hole-Cross

QITLKESGPTLVKPTQTLTLTCTFSGFSLSTSGVGVGWIRQPPGKALEWL
ALIYWDDDKRYSPSLKSRLTITKDTSKNQVVLTMTNMDPVDTATYYCAHH
KIERIFDYWGQGTLVTVSSASTAAPSVFIFPPSDEQLKSGTASVVCLLNN
FYPREAKVQWKVDNALQSGNSQESVTEQDSKDSTYSLSSTLTLSKADYEK
HKVYACEVTHQGLSSPVTKSFNRGECDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVCTLPPSRDELTKNQVSLSCAVKGFYPSDIAVEWESNGQPENNYKTT
PPVLDSDGSFFLVSKLTVDKSRWQQGNVFSCSVLHEALHSHYTQKSLSLS
PGK

SEQ ID NO: 236 is the amino acid sequence defining the 2-7 derived light chain of the 2-7/1-68 antibody 2-7VLCH1

QSALAQPASVSGSPGQSITISCTGTSSDVGAYNYVSWYQQHPGKAPKLMI
YDVSKRPSGVSNRFSGSKSGNTASLTISGLQAEDEADYYCSSYTTSSTVF
GGGTKLTVLSSASTKGPSVFPLAPSSKSTSGGTAALGCLVKDYFPEPVTV
SWNSGALTSGVHTFPAVLQSSGLYSLSSVVTVPSSSLGTQTYICNVNHKP
SNTKVDKKVEPKSC

SEQ ID NO: 237 is the amino acid sequence defining the 1-68 derived heavy chain of the 2-7/1-68 antibody 1-68HC-Knob

QVQLVQSGAEVKKPGASVKVSCKVSGYTLIELSMHWVRQAPGKGLEWMGG
FDPEDAETIYAQKFQGRVTMTEDTSTDTAYMELSSLRSEDTAVYYCATGW
AVAGSSDVWYYYYGMDVWGQGTTVTVSSASTKGPSVFPLAPSSKSTSGGT
AALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKA
KGQPREPQVYTLPPCRDELTKNQVSLWCLVKGFYPSDIAVEWESNGQPEN
NYKTTPPVLDSDGSFFLYSKLTVDKSRWQQGNVFSCSVLHEALHSHYTQK
SLSLSPGK

SEQ ID NO: 238 is the amino acid sequence defining the 1-68 derived light chain of the 2-7/1-68 antibody 1-68LC

QSALTQPPSVSGSPGQSVTISCTGTSSDVGSYNRVSWYQQPPGTAPKLMI
YEVSNRPSGVPDRFSGSKSGNTASLTISGLQAEDEADYYCSSYTSSSTYV
FGTGTKVTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECS

The 4-20/4-18 bispecific antibody sequence can comprise:

SEQ ID NO: 239 is the amino acid sequence defining the 4-20 derived heavy chain of the 4-20/4-18 antibody 4-20HC-Hole-Cross

QVQLLQSGAEVKKPGASVKVSCKASGYSFTSYYMHWVRQAPGQGLEWMGI
INPSGGGTTYAQKFQGRVTMTRDTSTSTVYMELSSLRSEDTAVYYCARPG
GGSYQEFDYWGQGTLVTVSSASTAAPSVFIFPPSDEQLKSGTASVVCLLN
NFYPREAKVQWKVDNALQSGNSQESVTEQDSKDSTYSLSSTLTLSKADYE
KHKVYACEVTHQGLSSPVTKSFNRGECDKTHTCPPCPAPELLGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVCTLPPSRDELTKNQVSLSCAVKGFYPSDIAVEWESNGQPENNYKT
TPPVLDSDGSFFLVSKLTVDKSRWQQGNVFSCSVLHEALHSHYTQKSLSL
SPGK

SEQ ID NO: 240 is the amino acid sequence defining the 4-20 derived light chain of the 4-20/4-18 antibody 4-20VLCH1

DIQMTQSPSSLSASVGDRVTITCRASQSISSYLNWYQQKPGKAPKLLISA
ASSLQSGVPSRFSGSGSGTDFTLTISSLQPEDFATYYCQQSYNTLQVTFG
GGTKVEIKSSASTKGPSVFPLAPSSKSTSGGTAALGCLVKDYFPEPVTVS
WNSGALTSGVHTFPAVLQSSGLYSLSSVVTVPSSSLGTQTYICNVNHKPS
NTKVDKKVEPKSC

SEQ ID NO: 241 is the amino acid sequence defining the 4-18 derived heavy chain of the 4-20/4-18 antibody 4-18HC-Knob

QVQLVESGGGVVQPGRSLRLSCAASGFTFSSYGMHWVRQAPGKGLEWVAV
ISYDGSNKHYADSVKGRFTISRDNSKNTLYLQMNSLRAEDTAVYYCAKDS
GYNYGYSWFDPWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPCRDELTKNQVSLWCLVKGFYPSDIAVEWESNGQPENNYKTTP
PVLDSDGSFFLYSKLTVDKSRWQQGNVFSCSVLHEALHSHYTQKSLSLSP
GK

SEQ ID NO: 242 is the amino acid sequence defining the 4-18 derived light chain of the 4-20/4-18 antibody 4-18LC

SYELTQPPSVSVSPGQTARITCSADALAKQYAYWYQQKPGQAPVLVIYKD
SERPSGIPERFSGSSSGTTVTLTISGVQAEDEADYYCQSTDNSGTYPNWV
FGGGTKLTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECS

The 2-15 mut/2-51 bispecific antibody sequence can comprise:

SEQ ID NO: 243 is the amino acid sequence defining the 2-15 mut derived heavy chain of the 2-15 mut/2-51 antibody 2-15HC-Hole-Cross

QVQLVQSGAEVKKPGASVRVSCKASGYTFTGYYMHWVRQAPGQGLEWMGW
INPISDGTNYAQKFQGWVTMTRDTSISTVYMELSRLRSDDTAVYYCARGG
SRCSGGNCYGWAYDAFDIWGQGTMITVSSASTAAPSVFIFPPSDEQLKSG
TASVVCLLNNFYPREAKVQWKVDNALQSGNSQESVTEQDSKDSTYSLSST
LTLSKADYEKHKVYACEVTHQGLSSPVTKSFNRGECDKTHTCPPCPAPEL
LGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEK
TISKAKGQPREPQVCTLPPSRDELTKNQVSLSCAVKGFYPSDIAVEWESN
GQPENNYKTTPPVLDSDGSFFLVSKLTVDKSRWQQGNVFSCSVLHEALHS
HYTQKSLSLSPGK

SEQ ID NO: 244 is the amino acid sequence defining the 2-15 mut derived Light chain of the 2-15 mut/2-51 antibody 2-15 mutVLCH1

QSALTQPASVSGSPGQSITISCTGTSSDVGGYNFVSWYQQHPGKAPKLMI
YDVSKRPSGVSNRFSGSKSGNTASLTISGLQAEDEADSYCSSYTSSSTFV
FGTGTKVTVLSSASTKGPSVFPLAPSSKSTSGGTAALGCLVKDYFPEPVT
VSWNSGALTSGVHTFPAVLQSSGLYS

SEQ ID NO: 245 is the amino acid sequence defining the 2-51 derived heavy chain of the 2-15 mut/2-51 antibody 2-51 HC-Knob

QVQLVQSGAEVKKPGASVKVSCKVSGYTLIELSMHWVRQAPGKGLEWMGG
FDPEDVETIYAQQFQGRVTMTEDTSTDTAYMELSSLRSEDTAVYYCATGW
AYKSTWYFGYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPCRDELTKNQVSLWCLVKGFYPSDIAVEWESNGQPENNYKTTPP
VLDSDGSFFLYSKLTVDKSRWQQGNVFSCSVLHEALHSHYTQKSLSLSPG
K

SEQ ID NO: 246 is the amino acid sequence defining the 2-51 derived light chain of the 2-15 mut/2-51 antibody 2-51LC

QSALTQPPSASGSPGQSVTISCTGTSSDVGGYNYVSWYQQHPGKAPKLMI
YEVSKRPSGVPDRFSGSKSGNTASLTVSGLQAEDEADYYCSSYAGSRMGF
GGGTKLTVLGQPKAAPSVTLFPPSSEELQANKATLVCLISDFYPGAVTVA
WKADSSPVKAGVETTTPSKQSNNKYAASSYLSLTPEQWKSHRSYSCQVTH
EGSTVEKTVAPTECS

The 4-20/4-8(39/51) bispecific antibody sequence can comprise:

SEQ ID NO: 247 is the amino acid sequence defining the 4-20 derived heavy chain of the 4-20/4-8(39/51) antibody 4-20HC-Hole-Cross

QVQLLQSGAEVKKPGASVKVSCKASGYSFTSYYMHWVRQAPGQGLEWMGI
INPSGGGTTYAQKFQGRVTMTRDTSTSTVYMELSSLRSEDTAVYYCARPG
GGSYQEFDYWGQGTLVTVSSASTAAPSVFIFPPSDEQLKSGTASVVCLLN
NFYPREAKVQWKVDNALQSGNSQESVTEQDSKDSTYSLSSTLTLSKADYE
KHKVYACEVTHQGLSSPVTKSFNRGECDKTHTCPPCPAPELLGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVCTLPPSRDELTKNQVSLSCAVKGFYPSDIAVEWESNGQPENNYKT
TPPVLDSDGSFFLVSKLTVDKSRWQQGNVFSCSVLHEALHSHYTQKSLSL
SPGK

SEQ ID NO: 248 is the amino acid sequence defining the 4-20 derived light chain of the 4-20/4-8(39/51) antibody 4-20VLCH1

DIQMTQSPSSLSASVGDRVTITCRASQSISSYLNWYQQKPGKAPKLLIS
AASSLQSGVPSRFSGSGSGTDFTLTISSLQPEDFATYYCQQSYNTLQVT
FGGGTKVEIKSSASTKGPSVFPLAPSSKSTSGGTAALGCLVKDYFPEPV
TVSWNSGALTSGVHTFPAVLQSSGLYSLSSVVTVPSSSLGTQTYICNVN
HKPSNTKVDKKVEPKSC

SEQ ID NO: 249 is the amino acid sequence defining the 4-8(39/51) derived heavy chain of the 4-20/4-8(39/51) antibody 4-8(39/51) HC-Knob

QVQLVQSGAEVKKAGSSVKVSCKASGGTFSSHTITWVRLAPGQGLEWMG
RNIPILGIANYAQKFQGRVTITADKSTSTAYMELSSLRSEDTAVYYCAS
LQTVDTAIEKYYGMDVWGQGTTVTVSSASTKGPSVFPLAPSSKSTSGGT
AALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPCRDELTKNQVSLWCLVKGFYPSDIAVEWESN
GQPENNYKTTPPVLDSDGSFFLYSKLTVDKSRWQQGNVFSCSVLHEALH
SHYTQKSLSLSPGK

SEQ ID NO: 250 is the amino acid sequence defining the 4-8(39/51) derived light chain of the 4-20/4-8(39/51) antibody 4-8LC

SYELTQPPSVSVSPGQTASITCSGDKLGDKYACWYQQKPGQSPVLVIYQ
DNKRPSGIPERFSGSNSGNTATLTISGTQAMDEADYYCQAWDSSTAVFG
GGTKLTVLGQPKAAPSVTLFPPSSEELQANKATLVCLISDFYPGAVTVA
WKADSSPVKAGVETTTPSKQSNNKYAASSYLSLTPEQWKSHRSYSCQVT
HEGSTVEKTVAPTECS

The 1-57/2-51 bispecific antibody sequence can comprise:

SEQ ID NO: 251 is the amino acid sequence defining the 1-57 derived heavy chain of the 1-57/2-51 antibody 1-57HC-Hole-Cross

EVHLVESGGGLVQPGGSLRLSCAASGFTFSDHYMDWVRQAPGKGLEWVG
RTRNKANSYTTEYAASVKGRFTISRDDAKNSLYLQMNSLKTEDTAVYYC
ARVHRWAYCINGVCFGAYSDYWGQGTLVTVSSASTAAPSVFIFPPSDEQ
LKSGTASVVCLLNNFYPREAKVQWKVDNALQSGNSQESVTEQDSKDSTY
SLSSTLTLSKADYEKHKVYACEVTHQGLSSPVTKSFNRGECDKTHTCPP
CPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNK
ALPAPIEKTISKAKGQPREPQVCTLPPSRDELTKNQVSLSCAVKGFYPS
DIAVEWESNGQPENNYKTTPPVLDSDGSFFLVSKLTVDKSRWQQGNVFS
CSVLHEALHSHYTQKSLSLSPGK

SEQ ID NO: 252 is the amino acid sequence defining the 1-57 derived light chain of the 1-57/2-51 antibody 1-57VLCH1

EIVLTQSPGTLSLSPGERATLSCRASQSVSSSYLAWYQQKPGQAPRLLI
YGASSRATGIPDRFSGSGSGTDFTLTISRLEPEDFAVYYCQQYGSSPST
FGQGTKLEIKSSASTKGPSVFPLAPSSKSTSGGTAALGCLVKDYFPEPV
TVSWNSGALTSGVHTFPAVLQSSGLYSLSSVVTVPSSSLGTQTYICNVN
HKPSNTKVDKKVEPKSC

SEQ ID NO: 253 is the amino acid sequence defining the 2-51 derived heavy chain of the 1-57/2-51 antibody 2-51 HC-Knob

QVQLVQSGAEVKKPGASVKVSCKVSGYTLIELSMHWVRQAPGKGLEWMG
GFDPEDVETIYAQQFQGRVTMTEDTSTDTAYMELSSLRSEDTAVYYCAT
GWAYKSTWYFGYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPCRDELTKNQVSLWCLVKGFYPSDIAVEWESNGQPE
NNYKTTPPVLDSDGSFFLYSKLTVDKSRWQQGNVFSCSVLHEALHSHYT
QKSLSLSPGK

SEQ ID NO: 254 is the amino acid sequence defining the 2-51 derived light chain of the 1-57/2-51 antibody 2-51LC

QSALTQPPSASGSPGQSVTISCTGTSSDVGGYNYVSWYQQHPGKAPKLM
IYEVSKRPSGVPDRFSGSKSGNTASLTVSGLQAEDEADYYCSSYAGSRM
GFGGGTKLTVLGQPKAAPSVTLFPPSSEELQANKATLVCLISDFYPGAV
TVAWKADSSPVKAGVETTTPSKQSNNKYAASSYLSLTPEQWKSHRSYSC
QVTHEGSTVEKTVAPTECS

The 4-20/1-87 bispecific antibody sequence can comprise:

SEQ ID NO: 255 is the amino acid sequence defining the 4-20 derived heavy chain of the 4-20/1-87 antibody 4-20HC-Hole-Cross

QVQLLQSGAEVKKPGASVKVSCKASGYSFTSYYMHWVRQAPGQGLEWMG
IINPSGGGTTYAQKFQGRVTMTRDTSTSTVYMELSSLRSEDTAVYYCAR
PGGGSYQEFDYWGQGTLVTVSSASTAAPSVFIFPPSDEQLKSGTASVVC
LLNNFYPREAKVQWKVDNALQSGNSQESVTEQDSKDSTYSLSSTLTLSK
ADYEKHKVYACEVTHQGLSSPVTKSFNRGECDKTHTCPPCPAPELLGGP
SVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTI
SKAKGQPREPQVCTLPPSRDELTKNQVSLSCAVKGFYPSDIAVEWESNG
QPENNYKTTPPVLDSDGSFFLVSKLTVDKSRWQQGNVFSCSVLHEALHS
HYTQKSLSLSPGK

SEQ ID NO: 256 is the amino acid sequence defining the 4-20 derived light chain of the 4-20/1-87 antibody 4-20VLCH1

DIQMTQSPSSLSASVGDRVTITCRASQSISSYLNWYQQKPGKAPKLLIS
AASSLQSGVPSRFSGSGSGTDFTLTISSLQPEDFATYYCQQSYNTLQVT
FGGGTKVEIKSSASTKGPSVFPLAPSSKSTSGGTAALGCLVKDYFPEPV
TVSWNSGALTSGVHTFPAVLQSSGLYSLSSVVTVPSSSLGTQTYICNVN
HKPSNTKVDKKVEPKSC

SEQ ID NO: 257 is amino acid sequence defining the 1-87 derived heavy chain of the 4-20/1-87 antibody 1-87HC-Knob

QVQLVQSGAEVKKPGASVKVSCKVSGYTLIELSMHWVRQAPGKGLEWMG
GFDPEDAETIYAQKFQGRVTMTEDTSTDTAYMELSSLRSEDTAVYYCAT
GIAVIGPPPSTYYYYGMDVWGQGTTVTVSSASTKGPSVFPLAPSSKSTS
GGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPI
EKTISKAKGQPREPQVYTLPPCRDELTKNQVSLWCLVKGFYPSDIAVEW
ESNGQPENNYKTTPPVLDSDGSFFLYSKLTVDKSRWQQGNVFSCSVLHE
ALHSHYTQKSLSLSPGK

SEQ ID NO: 258 is the amino acid sequence defining the 1-87 derived light chain of the 4-20/1-87 antibody 1-87LC

QSALTQPASVSGSPGQSITISCTGTSSDVGGYNYVSWYQQHPGKAPKLM
IYDVSKRPSGVSNRFSGSKSGNTASLTISGLQAEDEADYYCSSYTSSST
YVFGTGTKVTVLGQPKAAPSVTLFPPSSEELQANKATLVCLISDFYPGA
VTVAWKADSSPVKAGVETTTPSKQSNNKYAASSYLSLTPEQWKSHRSYS
CQVTHEGSTVEKTVAPTECS

The 1-57/1-68 bispecific antibody sequence can comprise:

SEQ ID NO: 259 is the amino acid sequence defining the 1-57 derived heavy chain of the 1-57/1-68 antibody 1-57HC-Hole-Cross

EVHLVESGGGLVQPGGSLRLSCAASGFTFSDHYMDWVRQAPGKGLEWVG
RTRNKANSYTTEYAASVKGRFTISRDDAKNSLYLQMNSLKTEDTAVYYC
ARVHRWAYCINGVCFGAYSDYWGQGTLVTVSSASTAAPSVFIFPPSDEQ
LKSGTASVVCLLNNFYPREAKVQWKVDNALQSGNSQESVTEQDSKDSTY
SLSSTLTLSKADYEKHKVYACEVTHQGLSSPVTKSFNRGECDKTHTCPP
CPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNK
ALPAPIEKTISKAKGQPREPQVCTLPPSRDELTKNQVSLSCAVKGFYPS
DIAVEWESNGQPENNYKTTPPVLDSDGSFFLVSKLTVDKSRWQQGNVFS
CSVLHEALHSHYTQKSLSLSPGK

SEQ ID NO: 260 is the amino acid sequence defining the 1-57 derived light chain of the 1-57/1-68 antibody 1-57VLCH1

EIVLTQSPGTLSLSPGERATLSCRASQSVSSSYLAWYQQKPGQAPRLLI
YGASSRATGIPDRFSGSGSGTDFTLTISRLEPEDFAVYYCQQYGSSPST
FGQGTKLEIKSSASTKGPSVFPLAPSSKSTSGGTAALGCLVKDYFPEPV
TVSWNSGALTSGVHTFPAVLQSSGLYSLSSVVTVPSSSLGTQTYICNVN
HKPSNTKVDKKVEPKSC

SEQ ID NO: 261 is the amino acid sequence defining the 1-68 derived heavy chain of the 1-57/1-68 antibody 1-68HC-Knob

QVQLVQSGAEVKKPGASVKVSCKVSGYTLIELSMHWVRQAPGKGLEWMG
GFDPEDAETIYAQKFQGRVTMTEDTSTDTAYMELSSLRSEDTAVYYCAT
GWAVAGSSDVWYYYYGMDVWGQGTTVTVSSASTKGPSVFPLAPSSKSTS
GGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPI
EKTISKAKGQPREPQVYTLPPCRDELTKNQVSLWCLVKGFYPSDIAVEW
ESNGQPENNYKTTPPVLDSDGSFFLYSKLTVDKSRWQQGNVFSCSVLHE
ALHSHYTQKSLSLSPGK

SEQ ID NO: 262 is the amino acid sequence defining the 1-68 derived light chain of the 1-57/1-68 antibody 1-68LC

QSALTQPPSVSGSPGQSVTISCTGTSSDVGSYNRVSWYQQPPGTAPKLM
IYEVSNRPSGVPDRFSGSKSGNTASLTISGLQAEDEADYYCSSYTSSST
YVFGTGTKVTVLGQPKAAPSVTLFPPSSEELQANKATLVCLISDFYPGA
VTVAWKADSSPVKAGVETTTPSKQSNNKYAASSYLSLTPEQWKSHRSYS
CQVTHEGSTVEKTVAPTECS

In some embodiments, a bispecific antibody can be generated from any combination of two monoclonal antibodies described herein. In some embodiments, the subject matter disclosed herein related to the DNA sequence encoding a bispecific antibody comprising any SEQ ID NO from 1-262 or a combination of SEQ ID NOs 1-262. In some embodiments, the subject matter disclosed herein related to the RNA sequence encoding a bispecific antibody comprising any SEQ ID NO from 1-262 or a combination of SEQ ID NOs 1-262.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Examples for Monoclonal Antibodies

### Example 1—Patient Antibody Response

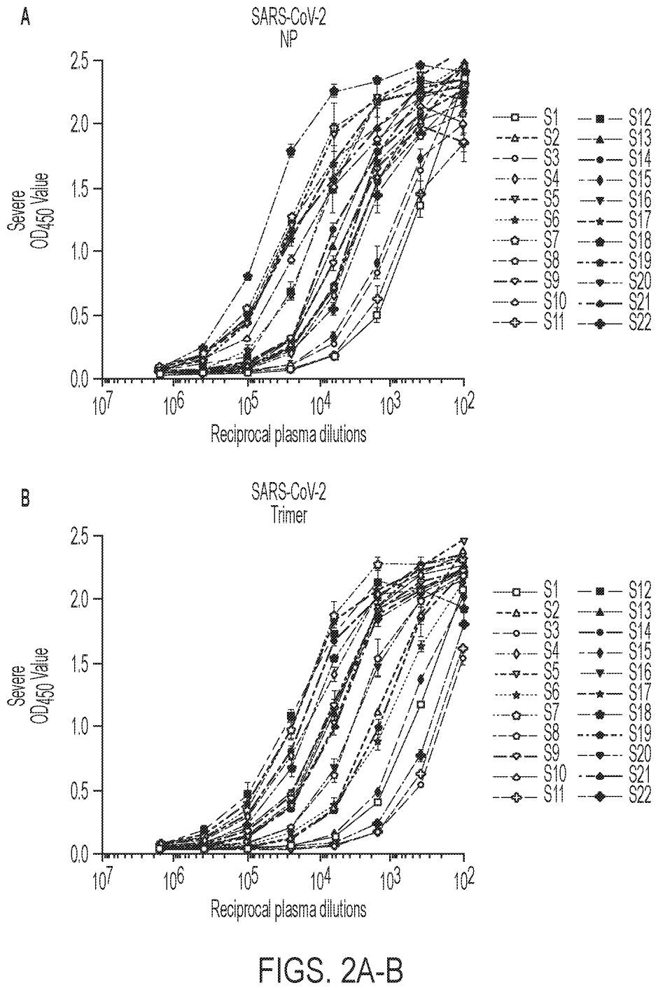

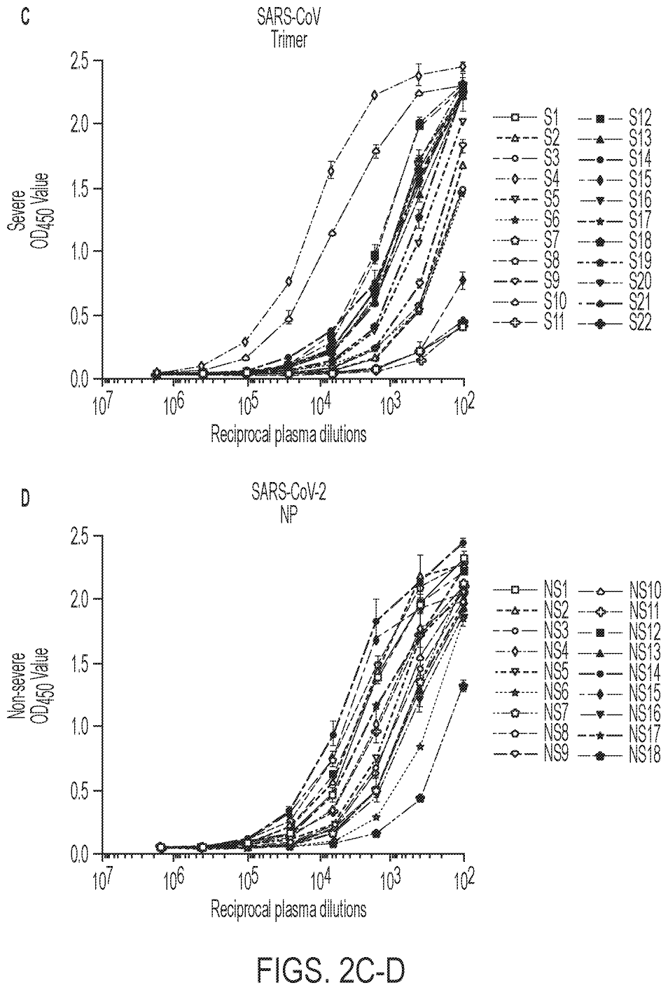

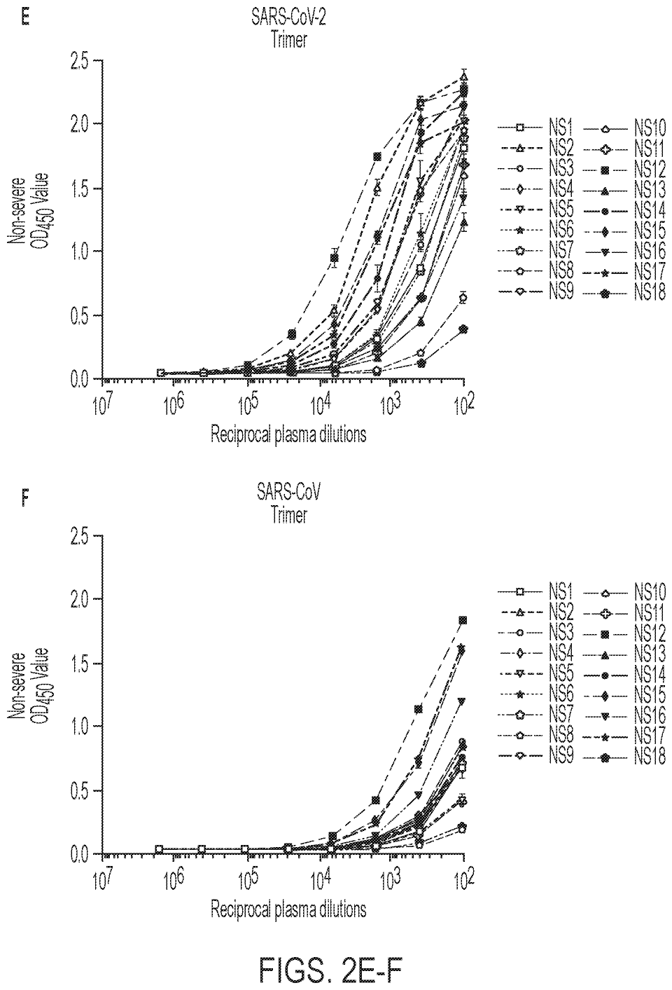

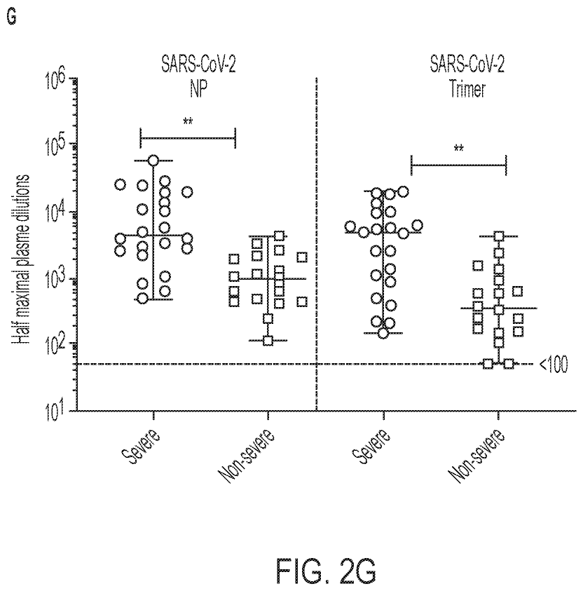

By studying 40 COVID-19 patient cases in great detail, it was found that patients with more severe disease develop more robust antibodies in their blood to the coronavirus. These antibodies include antibodies specific to the spike protein trimer. For the disease to be classified as severe, the COVID-19 patients need mechanical ventilation in an intensive care unit and treatment with hydroxychloroquine, remdesivir, and/or tocilizumab. For the disease to be classified as non-severe, the COVID-19 patients do not require intensive care. FIGS. 2A-G show ELISA binding data in which various COVID-19 patient plasma samples, indicated by different colored lines, were tested for their ability to bind to SARS-CoV-2 nucleocapsid antigen (FIGS. 2A and D), SARS-CoV-2 envelope trimer antigen (FIGS. 2B and E), or SARS-CoV envelope trimer antigen (FIGS. 2C and F). This binding assay is used to determine whether the plasma samples contain an element, most likely antibodies, that can interact with SARS-CoV-2 and SARS-CoV antigens, suggesting an immune response to the antigens. Those plasma samples that interact with both SARS-CoV-2 and SARS-CoV antigens indicate potential for utilizing the samples in neutralization of the antigens and the antigen carrying viruses. FIG. 2G shows a plot of the data from FIGS. 2A-F, which illustrates that higher SARS-CoV-2 antibody responses are present in plasma samples of patients with severe COVID-19 as compared to those patients with non-severe COVID-19.

### Example 2—Antibody Responses Correlated Potencies in Neutralizing SARS-CoV-2

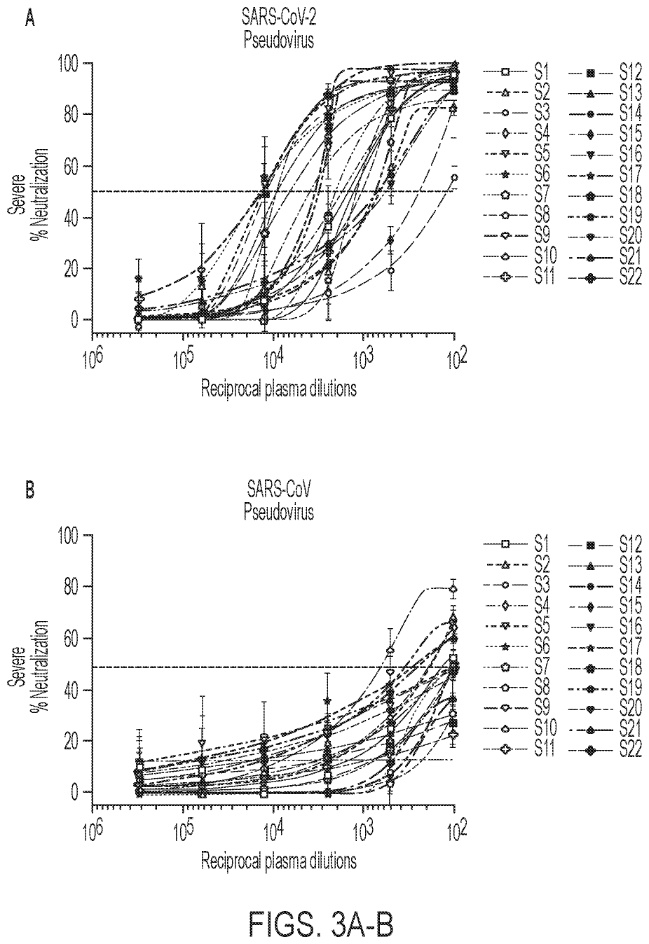

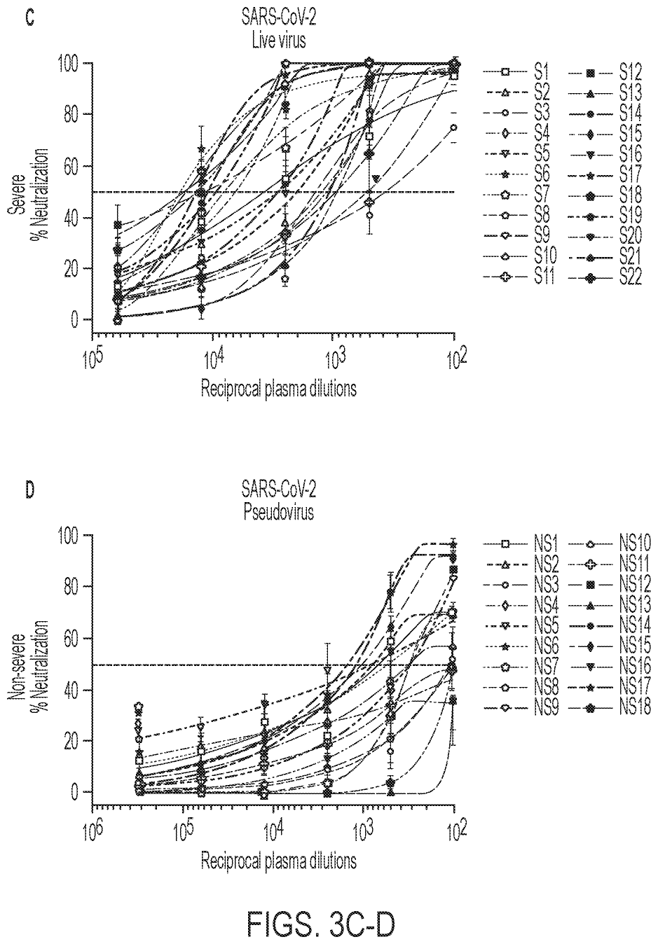

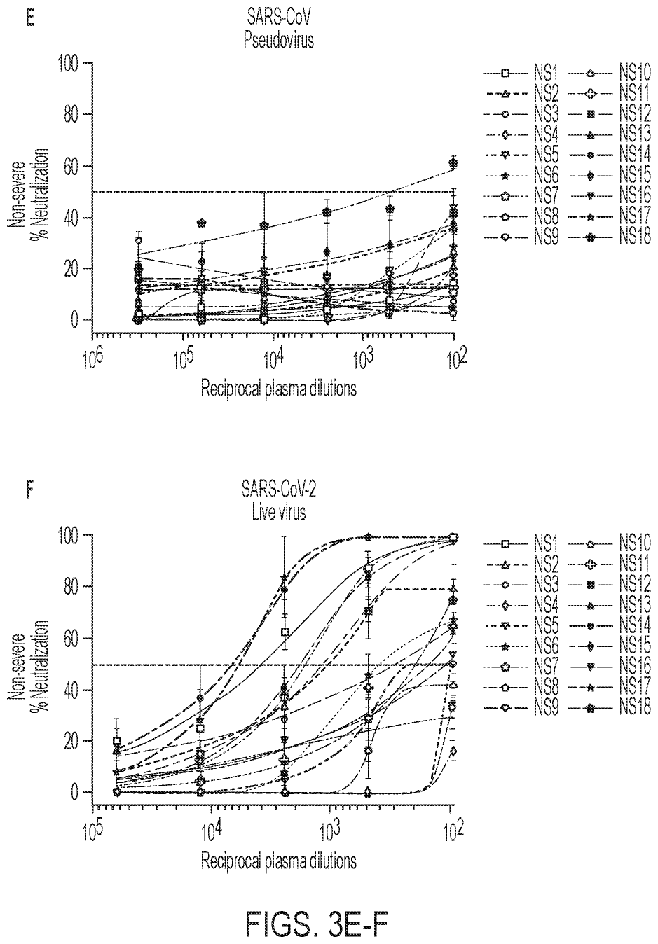

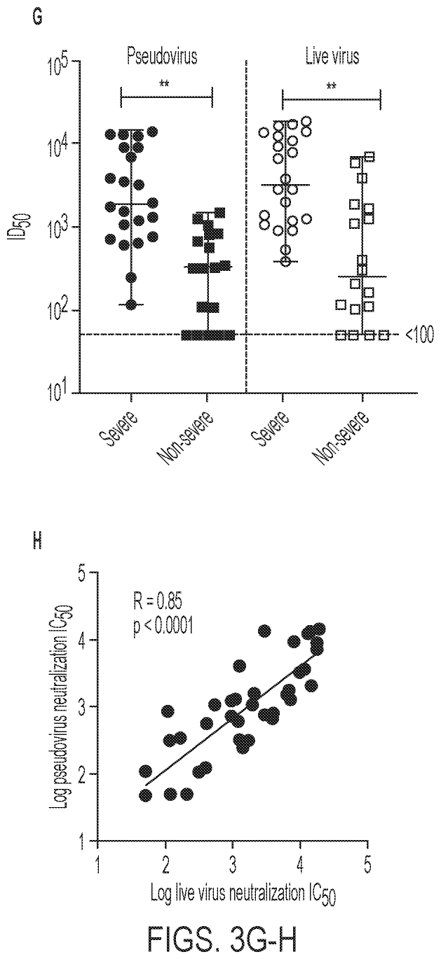

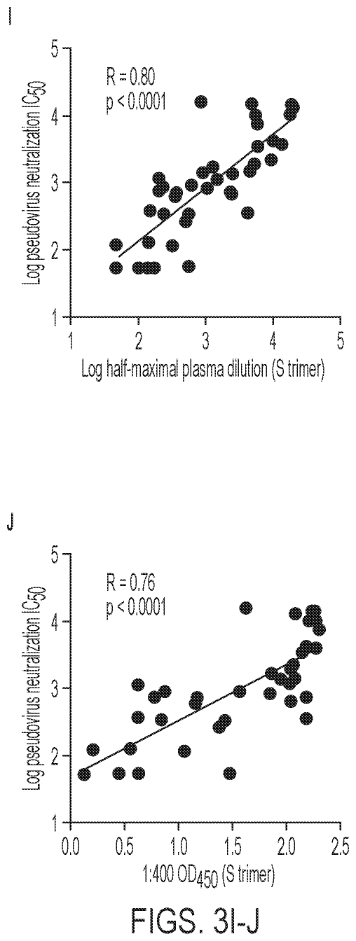

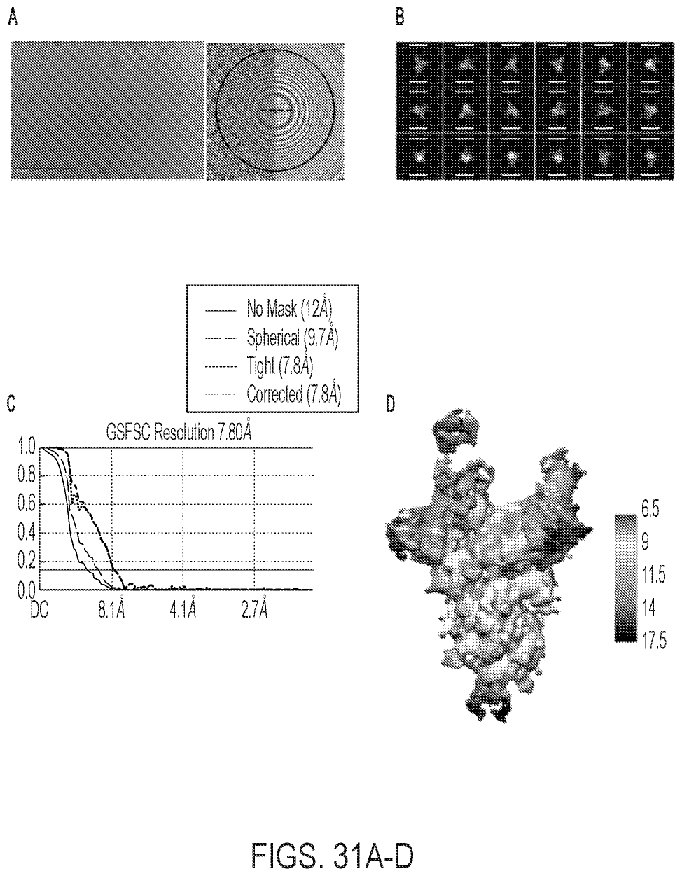

FIGS. 3A-J shows that stronger binding antibody responses correlate well with greater potencies in neutralizing SARS-CoV-2. Neutralization assays in which various COVID-19 patient plasma samples, indicated by different colored lines, were tested for their ability to protect the target cells from infection by SARS-CoV-2 in a single-cycle pseudovirus infectivity assay (FIGS. 3A and D), by SARS-CoV in a single-cycle pseudovirus infectivity assay (FIGS. 3B and E), and by SARS-CoV-2 in a replication competent live virus infectivity assay (FIGS. 3C and F). This assay is used to determine whether the plasma samples contain an element, most likely antibodies, that can effectively block SARS-CoV-2 or SARS-CoV infection. FIG. 3G shows plotting the dilution (ID₅₀) from FIGS. 3A-F at which the plasma inhibits 50% of virus infection. Higher SARS-CoV-2 inhibitory dilutions are present in plasma samples of patients with severe COVID-19, indicating a potentially more potent antibody response in these patients. FIGS. 3H-J show that the IC₅₀ values obtained by pseudovirus neutralization assay correlate well with the IC₅₀ values obtained from live virus neutralization assay (FIG. 311), the dilutions of plasmas achieving half of the readout of OD₄₅₀ values tested by ELISA in FIG. 2 (FIG. 31), and with the OD₄₅₀ values of the plasma binding to S trimer when diluted 400-fold in FIG. 2 (FIG. 3J). The data here indicates that the antibody binding ability to SARS-CoV-2 S trimer in the COVID-19 plasma samples are well correlated with the neutralization ability, suggesting that the single B cells sorted by SARS-CoV-2 S trimer from COVID-19 patients should have neutralization ability in their secreted antibodies.

### Example 3—Schematics for Identifying Potent Neutralizing Antibodies Against COVID-19

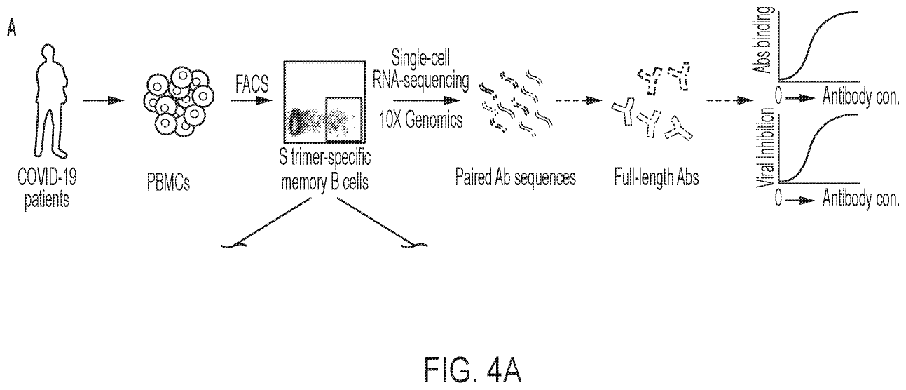

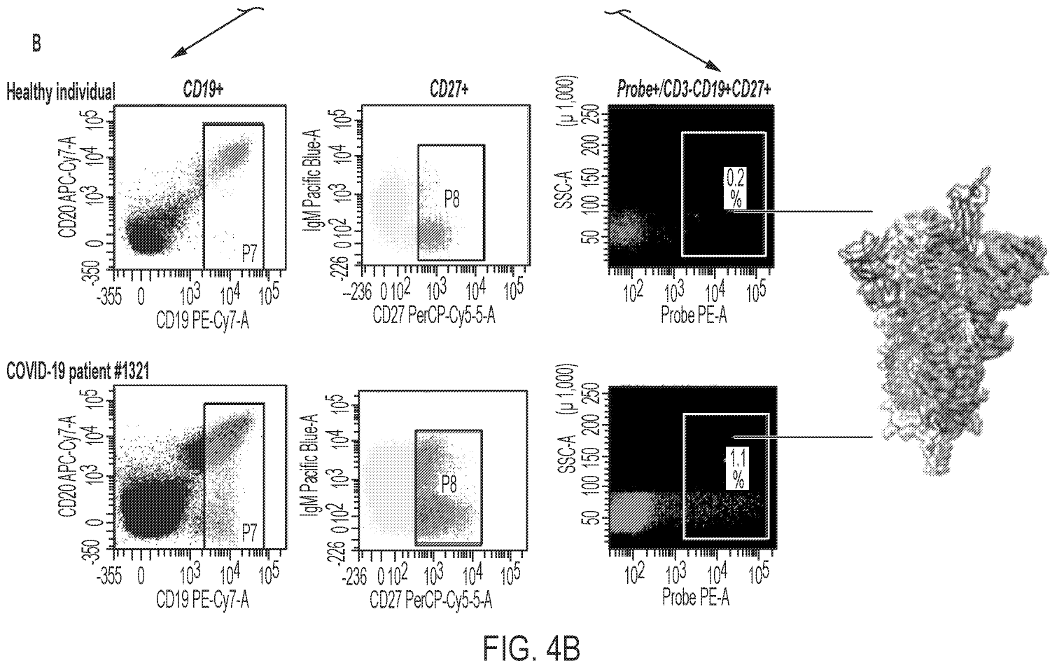

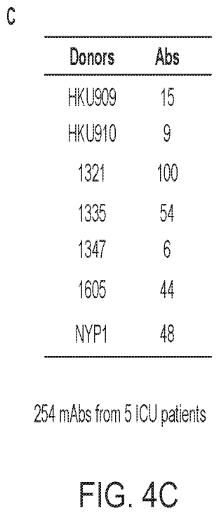

From the COVID-19 patient cases that indicated the most robust antibody responses in the experiments in FIGS. 2 and 3, blood sample was obtained and subjected to the experimental schema depicted in FIGS. 4A-C in order to identify monoclonal antibodies that could powerfully neutralize the SARS-CoV-2 virus. From each blood sample, the antibody-producing cells known as CD27+ memory B cells were isolated. In particular, the subset of cells that could bind the viral spike protein trimer were isolated.

The experimental protocol can include the following steps. PBMCs from infected individuals were collected and stained with LIVE/DEAD™ Fixable Yellow Dead Cell Stain Kit (Invitrogen, cat: L34959) at room temperature for 20 minutes to exclude dead cells. Then the cells were incubated with 10 μg/ml spike trimer firstly and further an antibody cocktail for identification of SARS-CoV-2 S trimer specific B cells. The cocktail consisted of CD3-PE-CF594(BD Biosciences, cat:562406), CD19-PE-cy7(Biolegend, cat:302216), CD20-APC-cy7(Biolegend, cat:302314), IgM-V450(BD Biosciences, cat:561286), CD27-PerCP-Cy5.5(BD Biosciences, cat:560612) and Anti-His-PE (Biolegend, cat:362603). Spiker trimer-specific single B cells were gated as CD3-CD19 CD27+Spike trimer+ and sorted into Eppendorf® LoBind microcentrifuge tubes (Sigma, cat: Z666505). After antigen-specific CD27+ memory B cell enrichment, 1310 B cells were loaded on 10X Chromium Chips. All of the experimental processes were performed before library preparation in a BSL3-level laboratory. Single-cell lysis and RNA first-strand synthesis was performed using 10X Chromium Single Cell 5′ Library & Gel Bead Kit according to the manufacturer's protocol. The following RNA and VDJ library preparation is performed according to the manufacturer's protocol (Chromium Single Cell V(D)J Reagent Kits, 10X Genomics) in a BSL-2 laboratory. Sequencing was performed on a Hiseq 2500 platform running Rapid SBS Kit V2 2×100 bp kit (Illumina), with a 26×91 pair-end reading mode. Armed with this information, each monoclonal antibody was reconstructed, now over 250 monoclonal antibodies are obtained by this method.

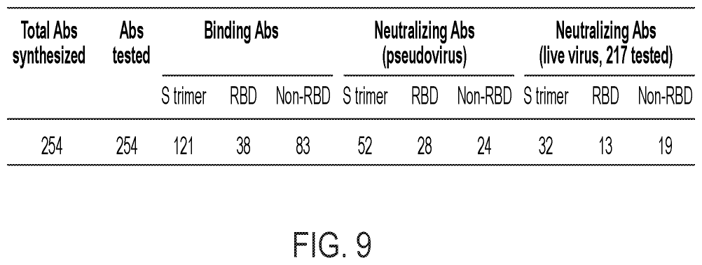

As shown in FIG. 9, there is a total of 254 monoclonal antibodies (mAbs) currently synthesized and tested. These include binding antibodies, pseudovirus neutralizing mAbs, and live virus neutralizing antibodies. The binding antibodies include 121 spike protein trimer targeting mAbs, 38 RBD-targeting mAbs, and 83 non-RBD-targeting mAbs. The pseudovirus neutralizing mAbs include 52 spike protein trimer-targeting mAbs, 28 RBD-targeting mAbs, and 24 non-RBD-targeting mAbs. The live virus neutralizing antibodies include 32 spike protein targeting mAbs, 13 RBD-targeting mAbs, and 19 non-RBD-targeting mAbs. 217 mAbs were tested for live virus neutralization.

### Example 4

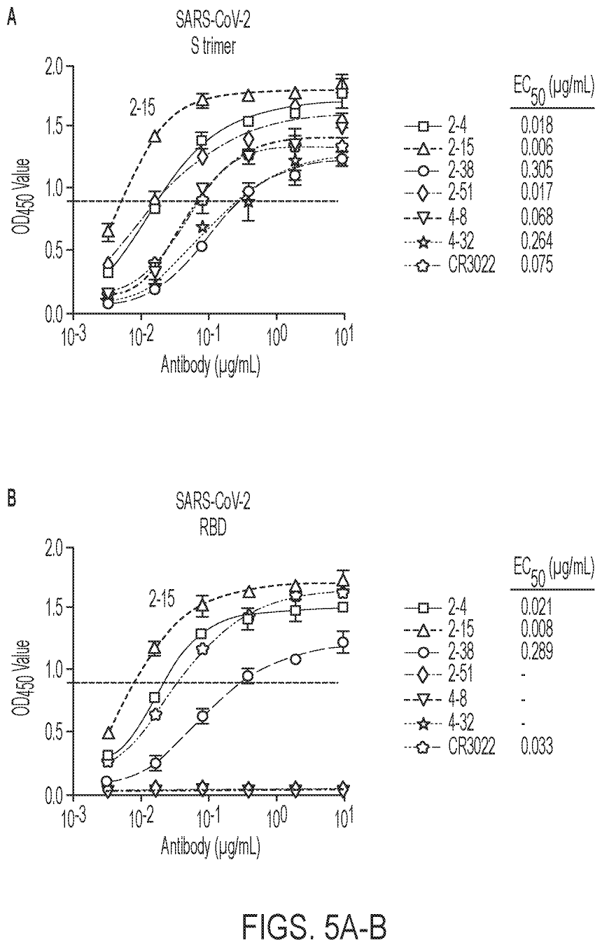

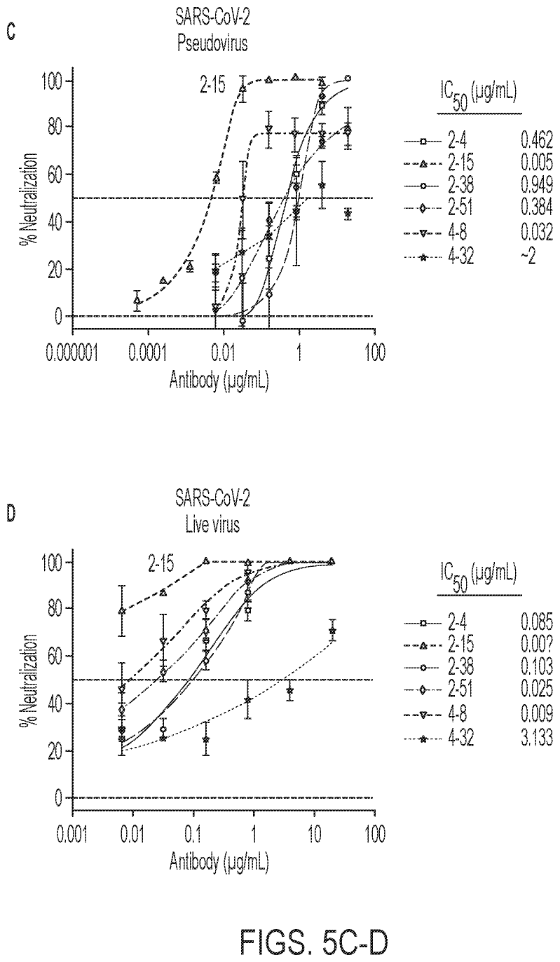

FIGS. 5A-D show characterization of the initially identified six monoclonal antibodies that were synthesized and purified, including the high binding ability to the SARS-CoV-2 S trimer (FIG. 5A) and to the virus receptor binding domain (FIG. 5B), and the neutralization capacity of the virus with high potency in both pseudovirus (FIG. 5C) and live replication competent virus (FIG. 5D) assays. Each colored line indicates a different monoclonal antibody. The half maximal effective concentration (EC₅₀) tested by ELISA and the half maximal inhibitory concentration (IC₅₀) tested by neutralization assay are presented to the side of each antibody. SARS-CoV antibody CR3022 is a control which can bind to but not neutralize SARS-CoV-2. These six antibodies are specific to SARS-CoV-2 S trimer, and three of them are RBD binders and three non-RBD binders. All of these six antibodies have neutralization activity against SARS-CoV-2 pseudovirus and live viruses and 2-15 is the most potent one. The binding data showed that these antibodies could be divided into at least two groups with different mechanisms in neutralizing the virus.

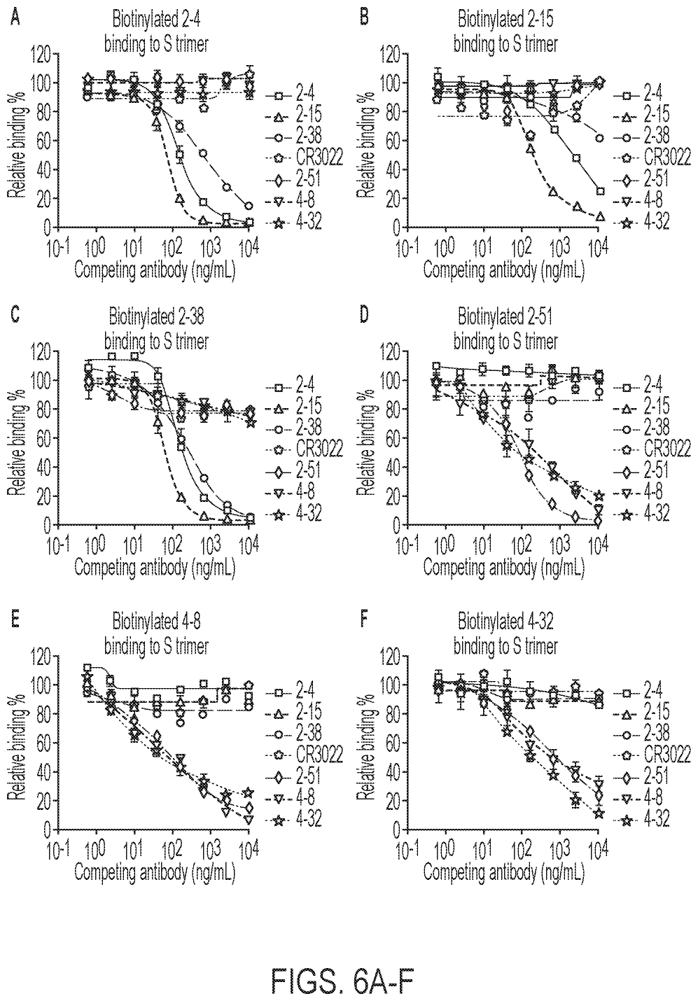

In order to determine where on the SAR-CoV-2 envelope these monoclonal antibodies bind, competition ELISA experiments were performed in order to map the epitopes of these new monoclonal antibodies. As shows in FIGS. 6A-F, each antibody was biotinylated, mixed with other serial diluted antibodies and then incubated with the spike protein (S) trimer. The binding of biotinylated antibodies to S trimer with the presence of other monoclonal antibodies was detected by ELISA. Each colored line represents a different monoclonal antibody. Six of the characterized monoclonal antibodies are categorized into two classes: RBD binding (FIGS. 6A-C) and non-RBD binding (FIGS. 6D-F). There is no competition between the two classes of antibodies but within the two classes, providing the possibility for combination of RBD and non-RBD binders against COVID-19 because they have different mechanisms of neutralizing the virus.

### Example 5

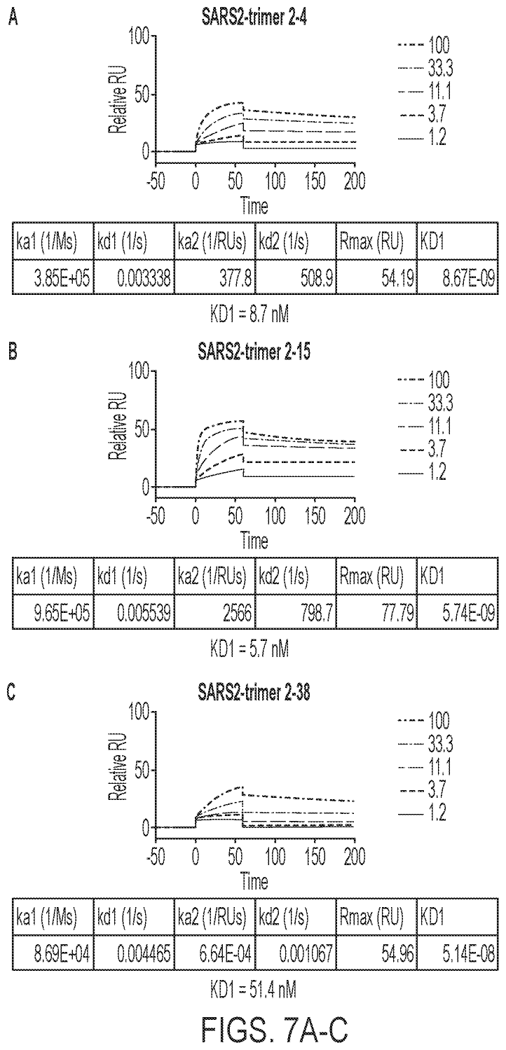

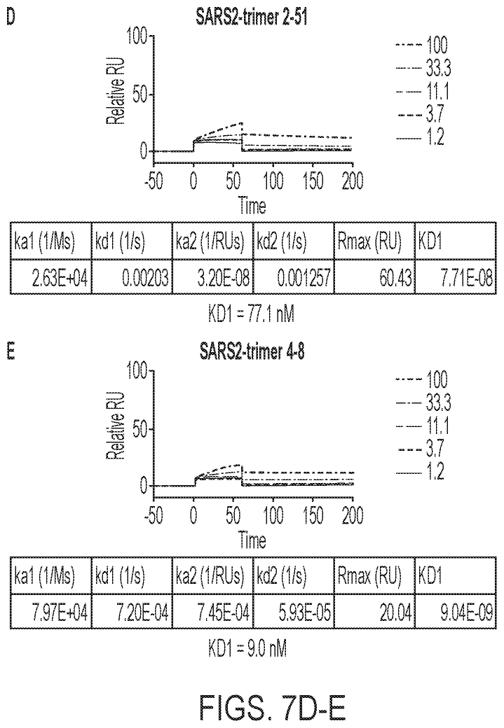

To further characterize the binding affinity of these new monoclonal antibodies, surface plasmon resonance experiments were performed to determine the binding affinity of the most promising monoclonal antibodies to the SARS-CoV-2 spike trimer. As shown in FIGS. 7A-E, each panel represents the binding affinity and binding kinetics of the indicated monoclonal antibody (i.e., 2-4, 2-15, 2-38, etc.) to the SARS-CoV-2 spike timer. The lower KD1 value indicates a higher antibody binding affinity. Colored lines indicate different dilutions of the respective antibody tested.

### Example 6

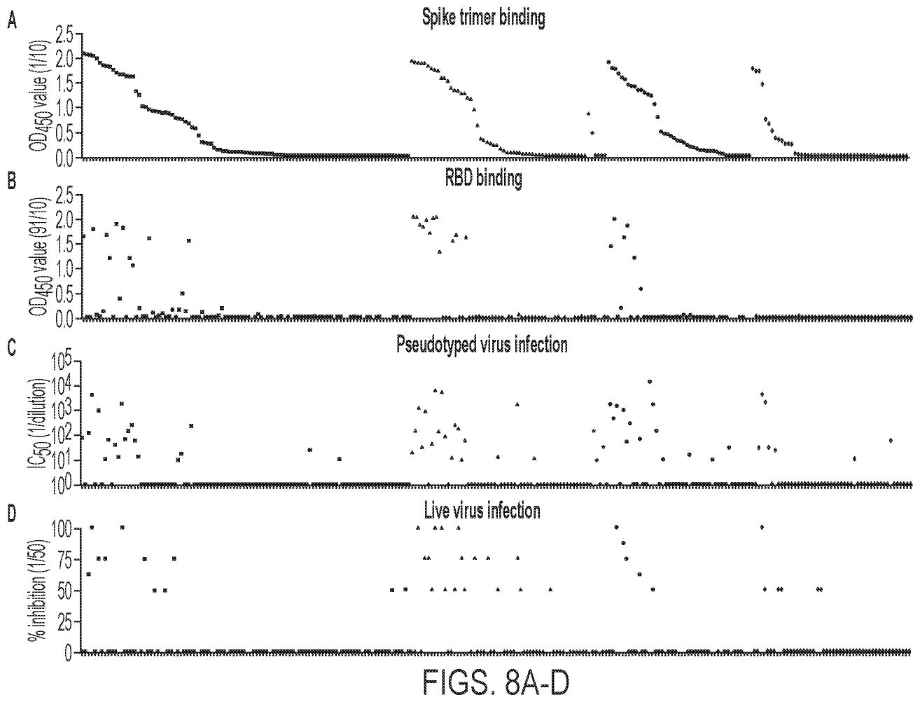

FIGS. 8A-D show initial screenings of mAbs that bind and neutralize the SARS-CoV-2 virus. FIG. 8A shows mAbs binding to spike trimer protein. FIG. 8B shows mAbs binding to the receptor-binding domain (RBD). FIG. 8C shows IC₅₀ values for pseudotyped virus infection. FIG. 8D shows percent inhibition values for live virus infection. FIG. 9 shows summary of synthesized antibodies.

### Example 7

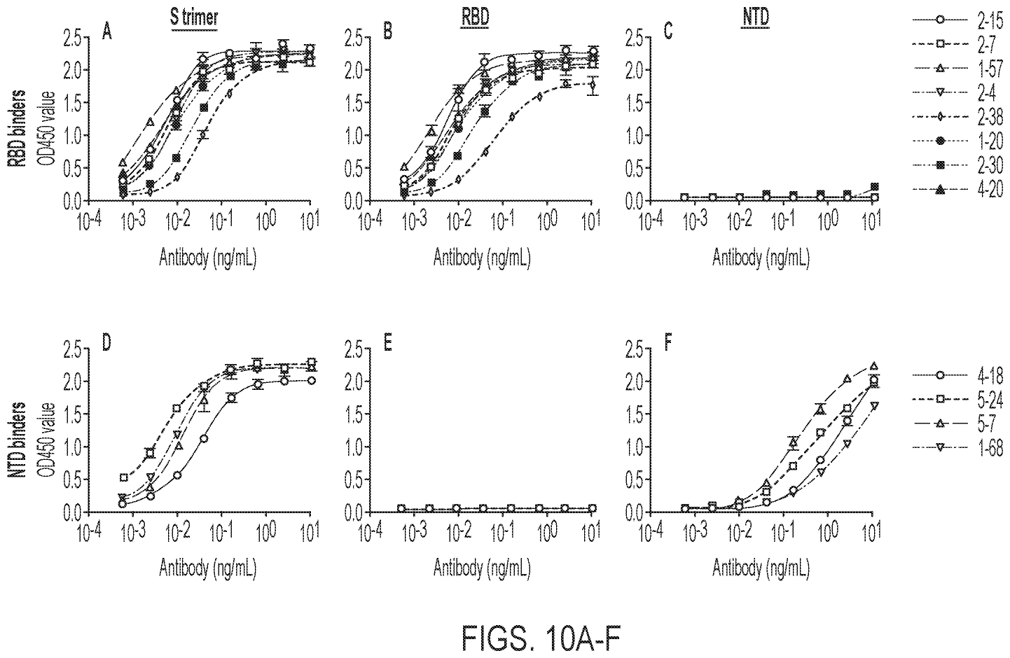

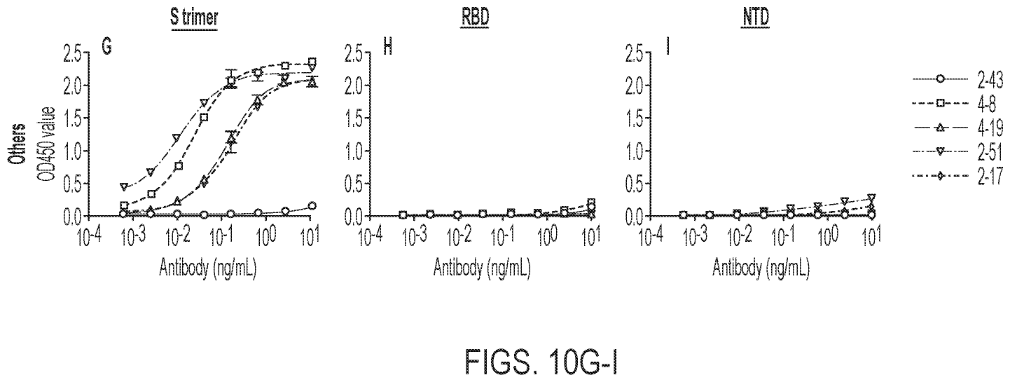

As shown in FIGS. 10A-I, the potent neutralizing mAbs disclosed herein can bind to the receptor binding domain of the spike protein trimer, to the N-terminal domain of the spike protein trimer or to a different epitope on the spike protein trimer. Each colored line shown in FIGS. 10A-I indicates a different monoclonal antibody utilized in an ELISA binding assay. FIGS. 10A, D, and G show binding to spike protein trimer. FIGS. 10B, E, and H show binding to the RBD. FIGS. 10C, F, and I show binding to the NTD. FIGS. 10A-C show binding of mAbs, which potently bind the RBD. FIGS. 10D-F show binding of mAbs, which potently bind the NTD. FIGS. 10G-I show binding of mAb, which bind epitopes other than the RBD or the NTD. Monoclonal antibodies 2-15, 2-7, 1-57, 2-4, 2-38, 1-20, 2-30, and 4-20 specifically target the RBD of the spike protein trimer. They show no binding affinity for the NTD of the spike protein trimer as shown in FIG. 10C. Monoclonal antibodies 4-18, 5-24, 5-7, and 1-68 specifically target the NTD of the spike protein trimer. These antibodies show no binding affinity for the RBD of the spike protein trimer as shown in FIG. 10E. Monoclonal antibodies 2-43, 4-8, 4-19, 2-51, and 2-17 target at least one other epitope on the spike protein trimer. These antibodies do not show binding affinity for the RBD nor for the NTD as shown in FIGS. 10H and I, respectively.

### Example 8

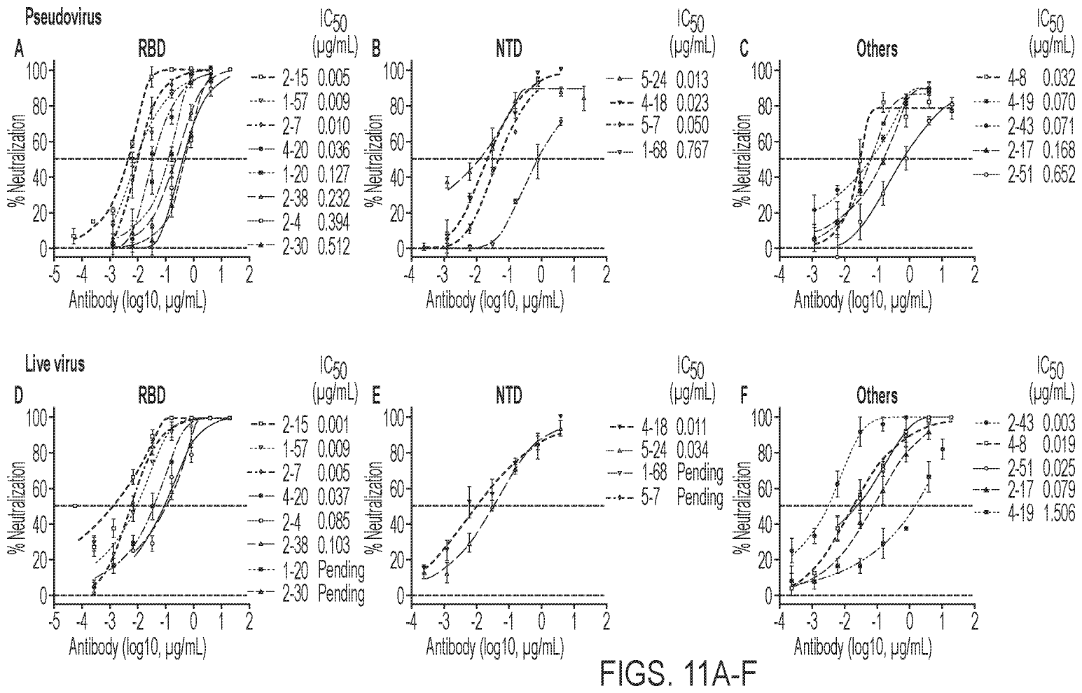

FIGS. 11A-F show SARS-CoV-2 neutralization activity of select mAbs. FIG. 11A shows neutralization activity of RBD-targeting mAbs neutralizing a pseudovirus infection. The range of IC₅₀ concentrations for these antibodies is from 0.005 μg/ml to 0.512 μg/ml. FIG. 11B shows neutralization activity of NTD-targeting mAbs neutralizing a pseudovirus infection. The range of IC₅₀ concentrations for these antibodies is from 0.013 μg/ml to 0.767 μg/ml. FIG. 11C shows neutralization activity of mAbs, which target epitopes other than the RBD or the NTD, neutralizing a pseudovirus infection. The range of IC₅₀ concentrations for these antibodies is from 0.070 μg/ml to 0.652 μg/ml. FIG. 11D shows neutralization activity of RBD-targeting mAbs neutralizing a live virus infection. The range of IC₅₀ concentrations for these antibodies is from 0.001 μg/ml to 0.103 μg/ml. FIG. 11E shows neutralization activity of NTD-targeting mAbs neutralizing a live virus infection. The range of IC₅₀ concentrations for these antibodies is from 0.011 μg/ml to 0.034 μg/ml. FIG. 11F shows neutralization activity of mAbs, which target epitopes other than the RBD or the NTD, neutralizing a live virus infection. The range of IC₅₀ concentrations for these antibodies is from 0.003 μg/ml to 0.079 μg/ml.

### Example 9

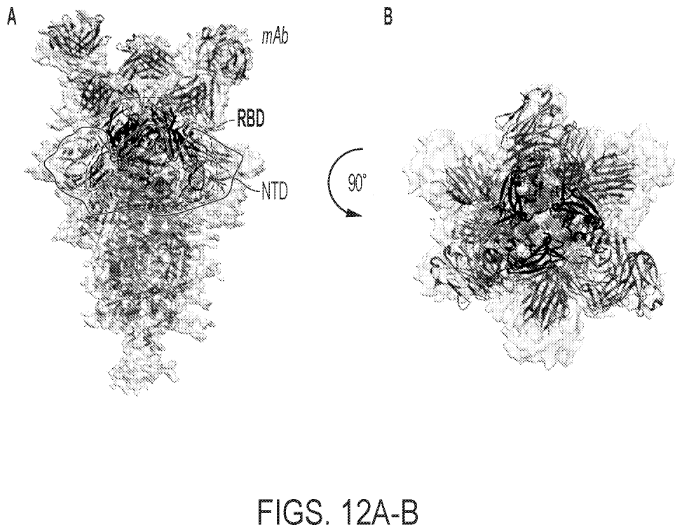

FIGS. 12A-B show a CryoEM structure of an RBD-targeting monoclonal antibody. FIG. 12A shows a side view of the structure while FIG. 12B shows a top view of the structure. The mAb is shown in blue ribbon directly adjacent to the RBD, which is shown in green ribbon. The CryoEM structure suggests that the mAb targeting the RBD does not directly interact with the NTD, which is shown in brown ribbon.

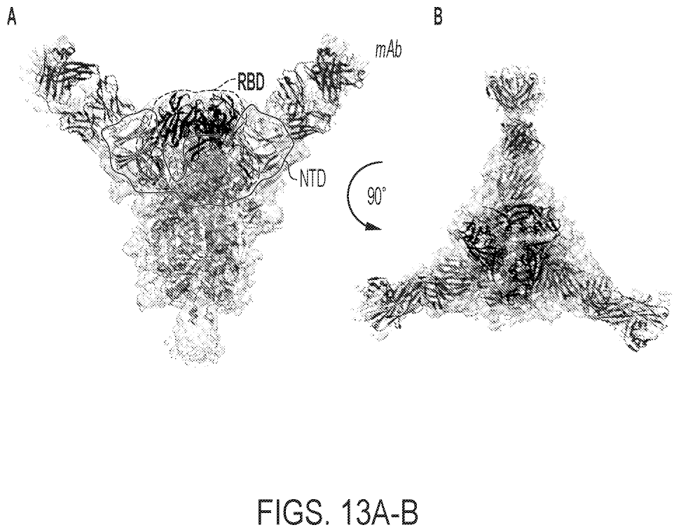

FIGS. 13A-B show a CryoEM structure of an NTD-targeting monoclonal antibody. FIG. 13A shows a side view of the structure while FIG. 13B shows a top view of the structure. The mAb is shown in blue ribbon directly adjacent to the NTD, which is shown in brown ribbon. The CryoEM structure suggests that the mAb targeting the NTD does not directly interact with the RBD, which is shown in green ribbon.

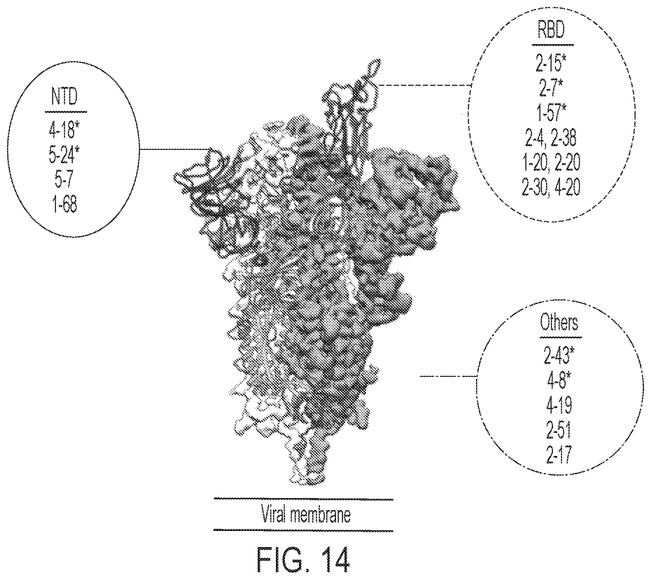

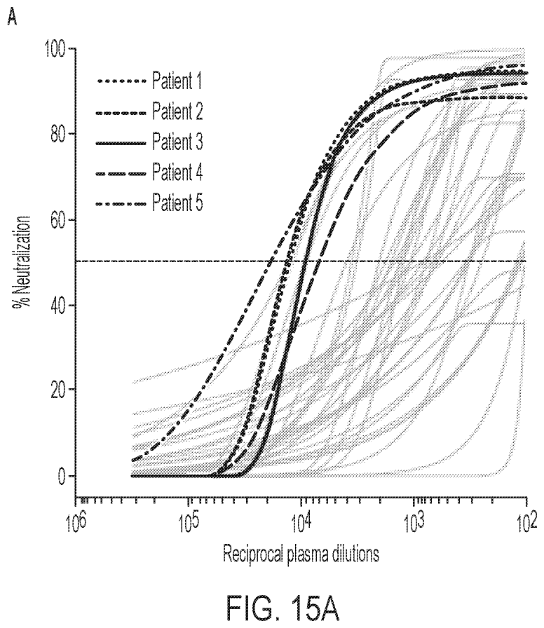

FIG. 14 shows three SARS-CoV-2 neutralizing epitope clusters identified on the spike protein trimer. The NTD cluster is shown in blue ribbon. The RBD is shown in green ribbon. Other color indicate potential neutralizing epitopes on the spike protein, which are different from the NTD and RBD clusters.

### Example 10—Potent Neutralizing Monoclonal Antibodies Directed to Multiple Epitopes on the SARS-CoV-2 Spike

Abstract

The SARS-CoV-2 pandemic rages on with devastating consequences on human lives and the global economy. The discovery and development of virus-neutralizing monoclonal antibodies could be one approach to treat or prevent infection by this novel coronavirus. Here we report the isolation of 61 SARS-CoV-2-neutralizing monoclonal antibodies from 5 infected patients hospitalized with severe disease. Among these are 19 antibodies that potently neutralized the authentic SARS-CoV-2 in vitro, 9 of which exhibited exquisite potency, with 50% virus-inhibitory concentrations of 1 to 9 ng/mL. Epitope mapping showed this collection of 19 antibodies to be about equally divided between those directed to the receptor-binding domain (RBD) and those to the N-terminal domain (NTD), indicating that both of these regions at the top of the viral spike are quite immunogenic. In addition, two other powerful neutralizing antibodies recognized quaternary epitopes that are overlapping with the domains at the top of the spike. Cryo-electron microscopy structures of one antibody targeting RBD, a second targeting NTD, and a third bridging RBD and NTD revealed recognition of the closed, “all RBD-down” conformation of the spike. Several of these monoclonal antibodies are promising candidates for clinical development as potential therapeutic and/or prophylactic agents against SARS-CoV-2.

### BACKGROUND

A novel coronavirus, now termed SARS-CoV-2^{1,2}, has caused nearly 8 million confirmed infections globally, leading to about 450,000 deaths. This pandemic has also put much of the world on pause, with unprecedented disruption of lives and unparalleled damage to the economy. A return to some semblance of normalcy will depend on science to deliver an effective solution, and the scientific community has responded admirably. Drug development is well underway, and vaccine candidates are entering clinical trials. Another promising approach is the isolation of SARS-CoV-2-neutralizing monoclonal antibodies (mAbs) that could be used as therapeutic or prophylactic agents. The primary target for such antibodies is the viral spike, a trimeric protein^{3,4} that is responsible for binding to the ACE2 receptor on the host cell^{1,3,5,6}. The spike protein is comprised of two subunits. The S1 subunit has two major structural elements: RBD and NTD; the S2 subunit mediates virus-cell membrane fusion after the RBD engages ACE2. Reports of discovery of neutralizing mAbs that target the RBD have been published recently⁷⁻¹¹. We now describe our efforts in isolating and characterizing a collection of mAbs that not only target multiple epitopes on the viral spike but also show exquisite potency in neutralizing SARS-CoV-2.

Patient Selection

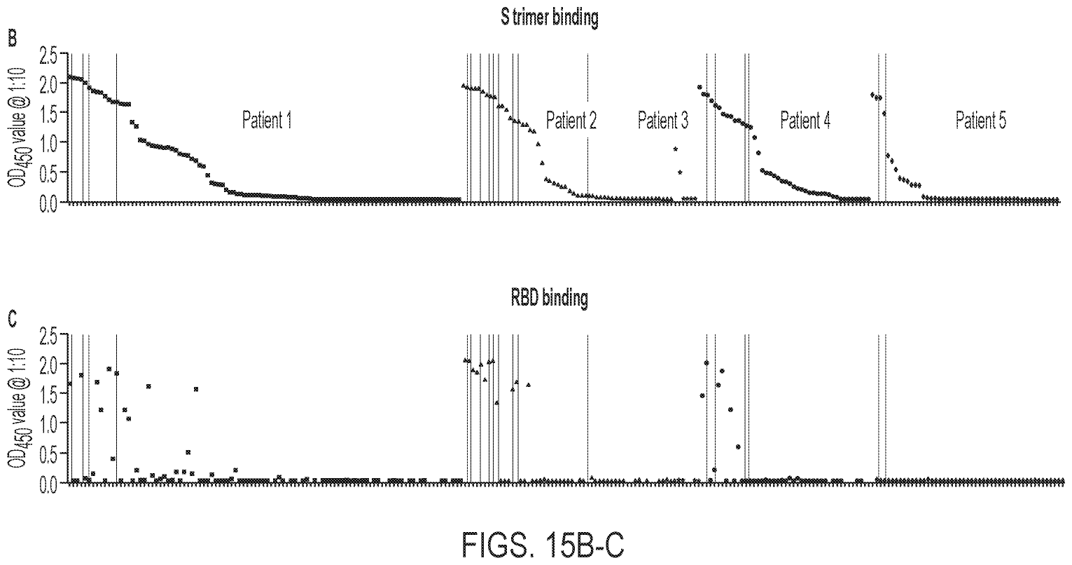

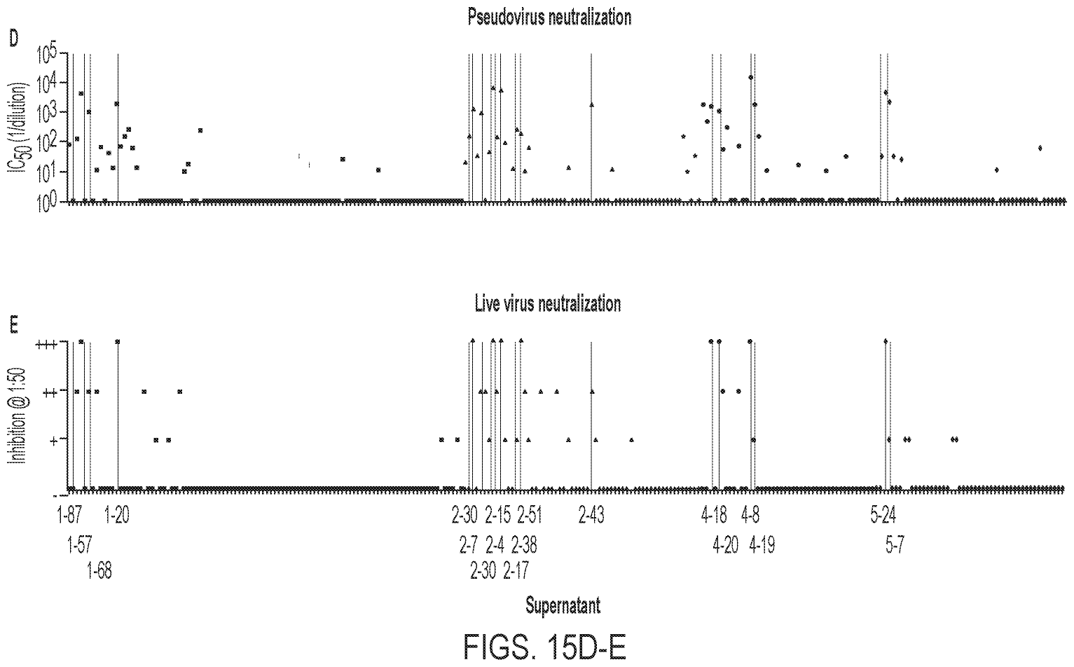

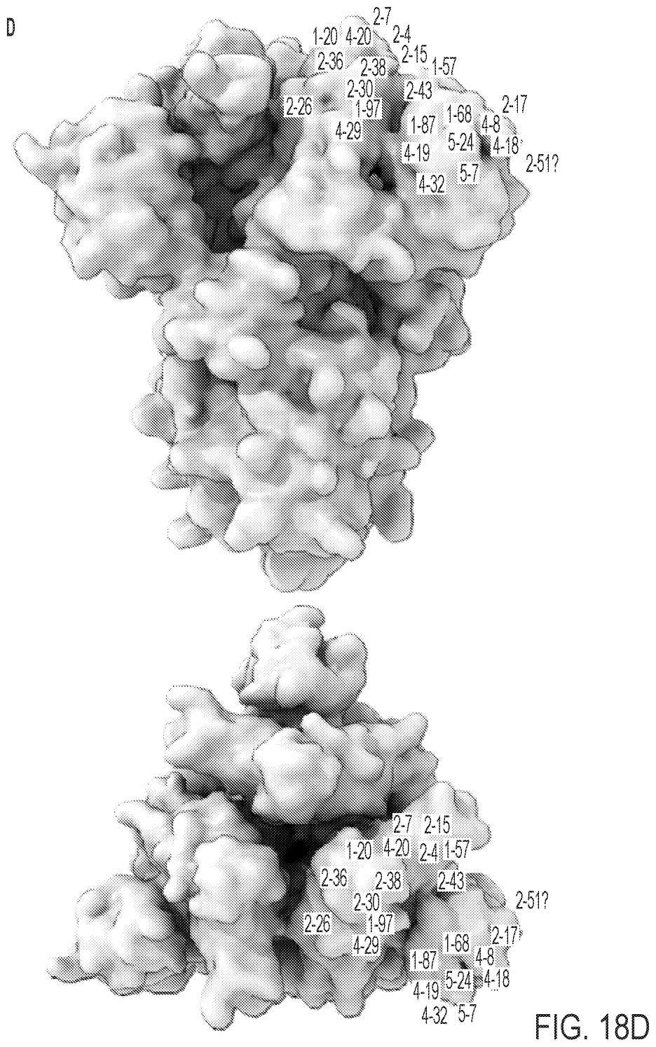

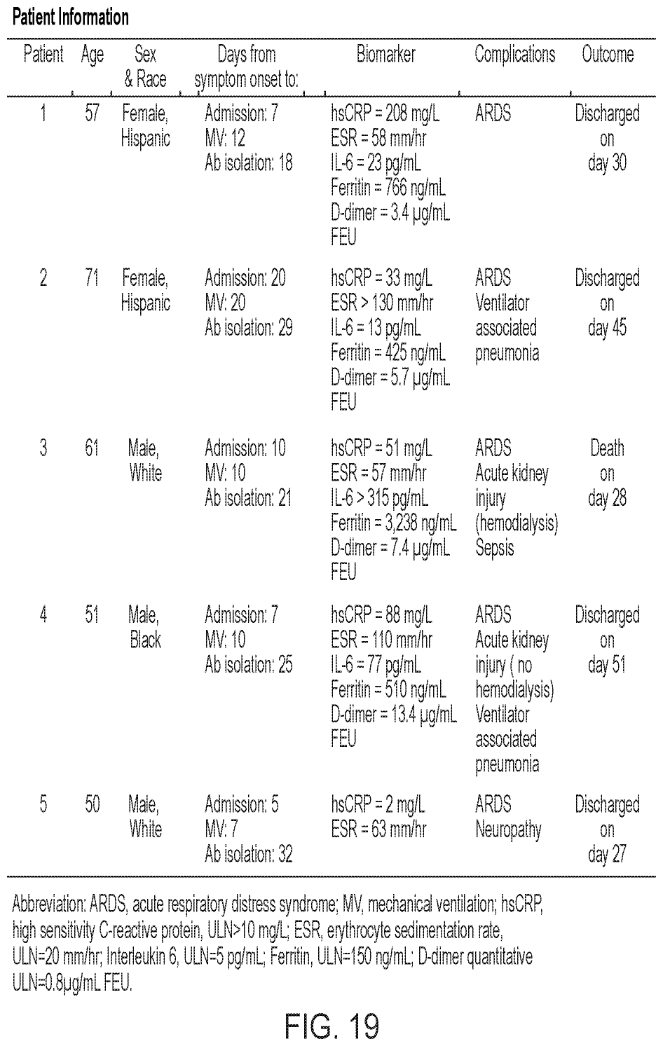

Forty patients with PCR-confirmed SARS-CoV-2 infection were enrolled in an observational cohort study on virus-neutralizing antibodies. Plasma samples from all subjects were first tested for neutralizing activity against SARS-CoV-2 pseudovirus (Wuhan-Hu-1 spike pseudotyped with vesicular stomatitis virus). Widely varying neutralizing titers were observed, with IC50 ranging from a reciprocal plasma dilution of <100 to ˜13,000 (FIG. 15A). Five patients were chosen for mAb isolation because their plasma virus-neutralizing titers were among the highest. The clinical characteristics of these 5 cases are summarized in FIG. 19. In brief, all were severely ill with acute respiratory distress syndrome requiring mechanical ventilation. Their ages ranged from 50 to 71. Two were Hispanic females, two were white males, and one was a black male. One patient died, while the others recovered. Importantly, blood for isolation of SARS-CoV-2 mAbs was obtained on day 18 to 32 post onset of symptoms.

Monoclonal Antibody Isolation and Construction

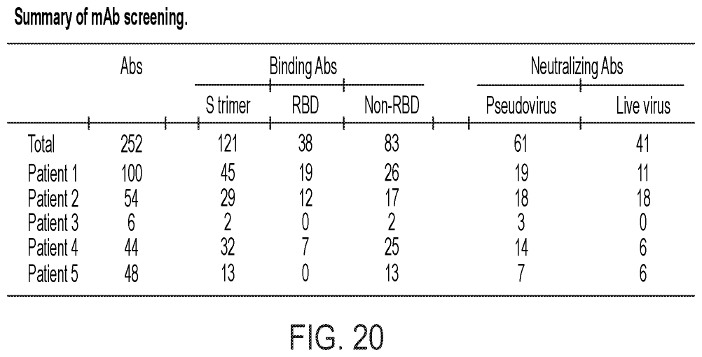

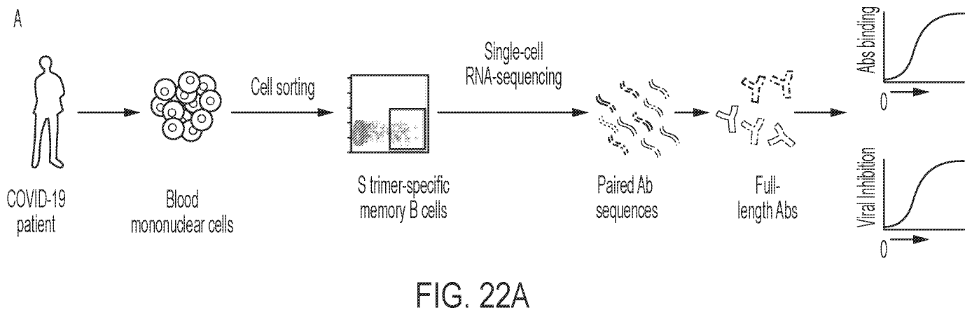

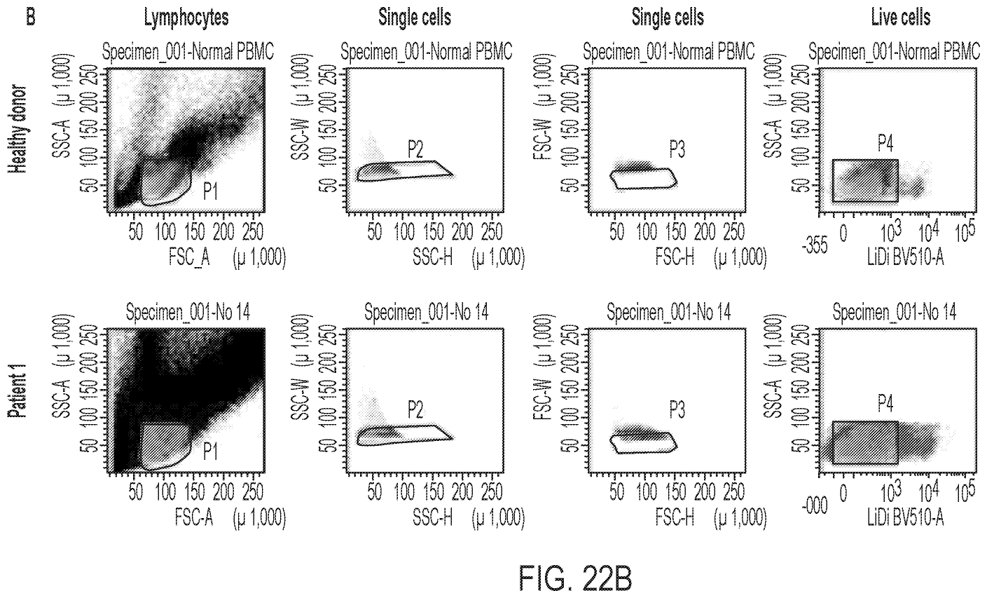

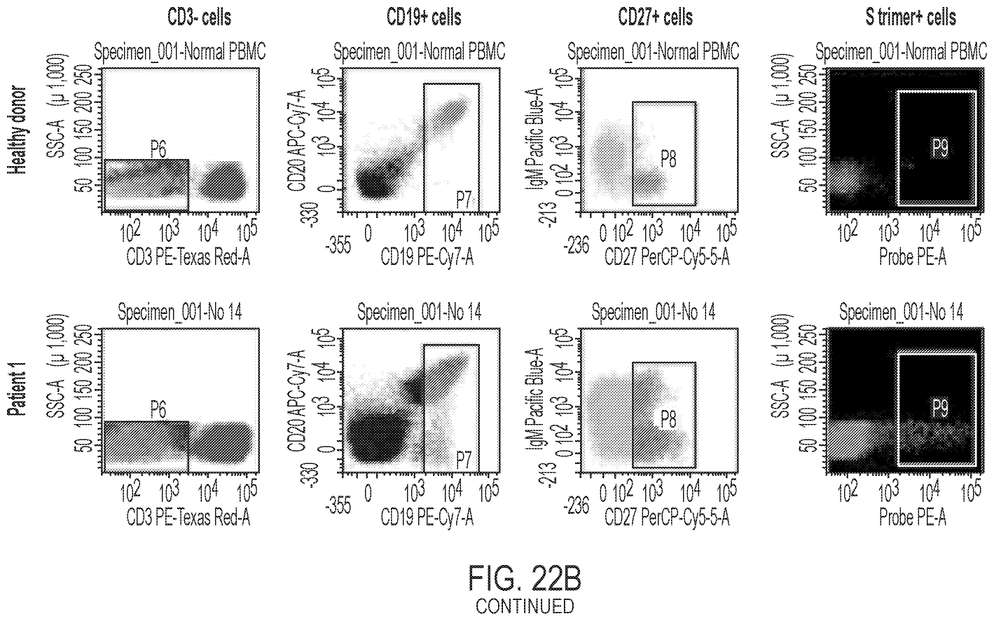

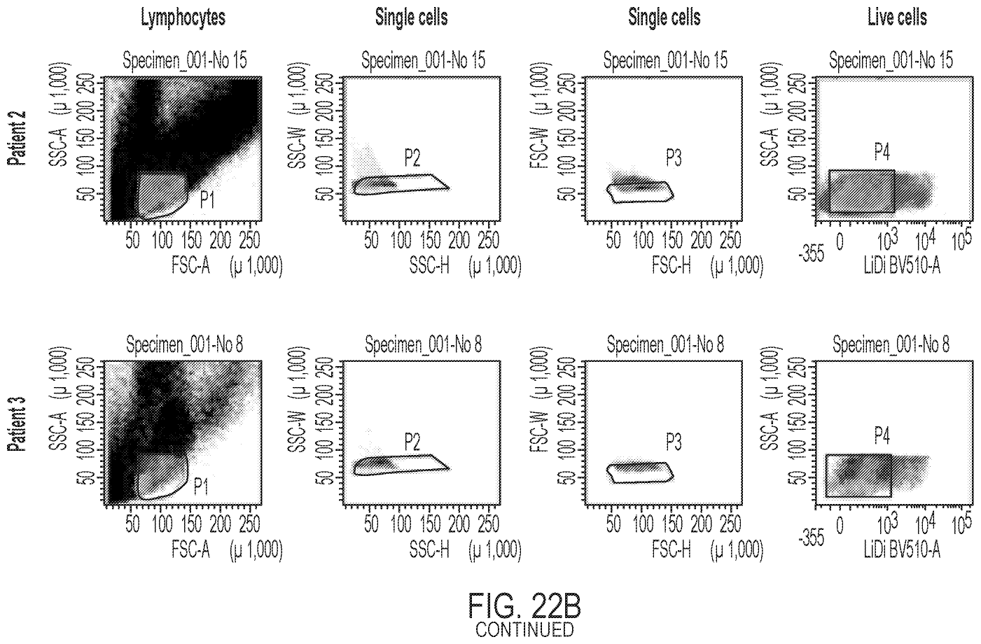

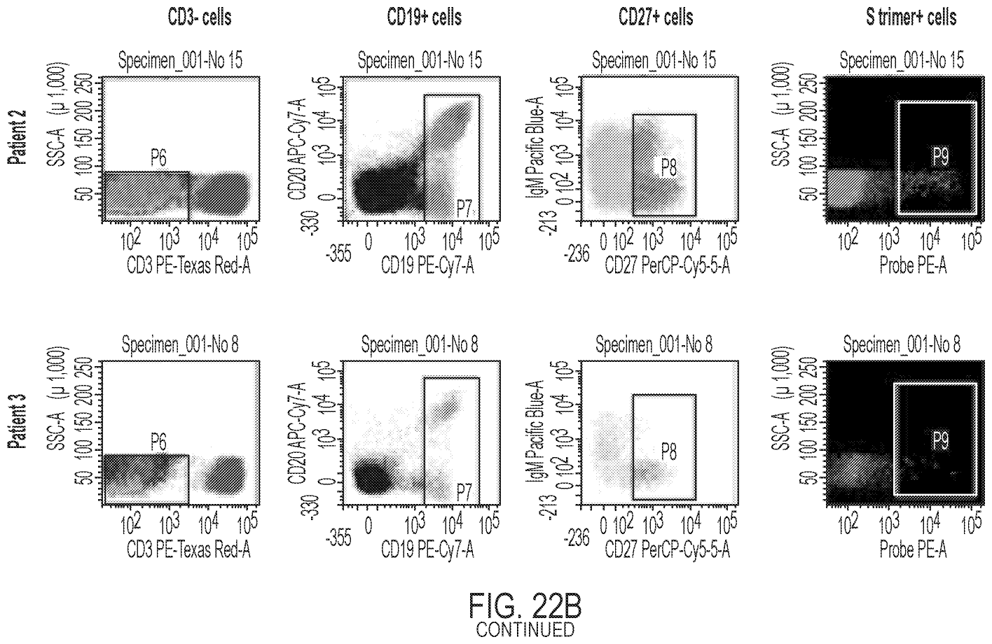

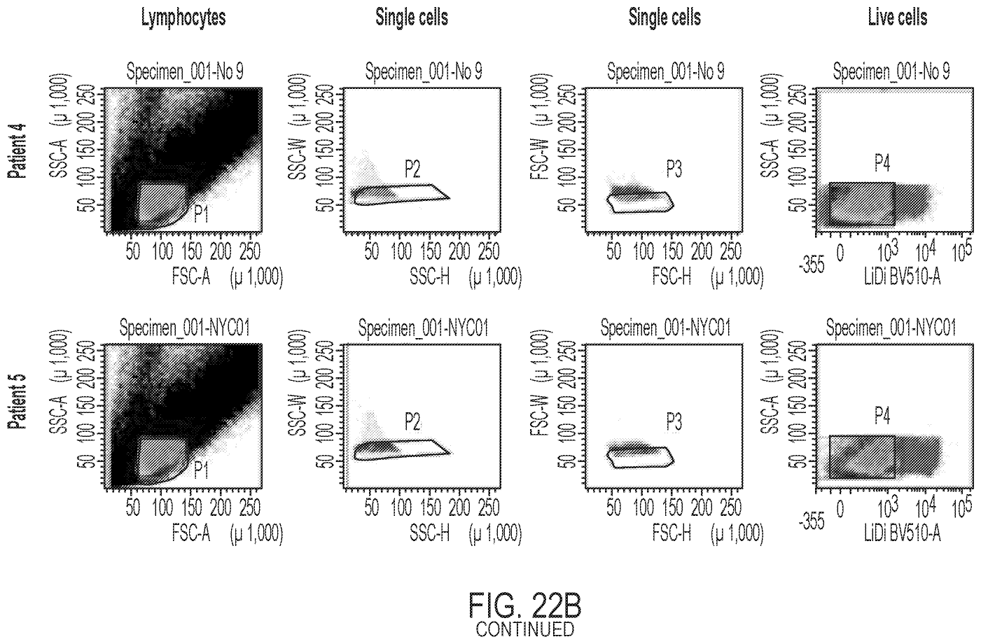

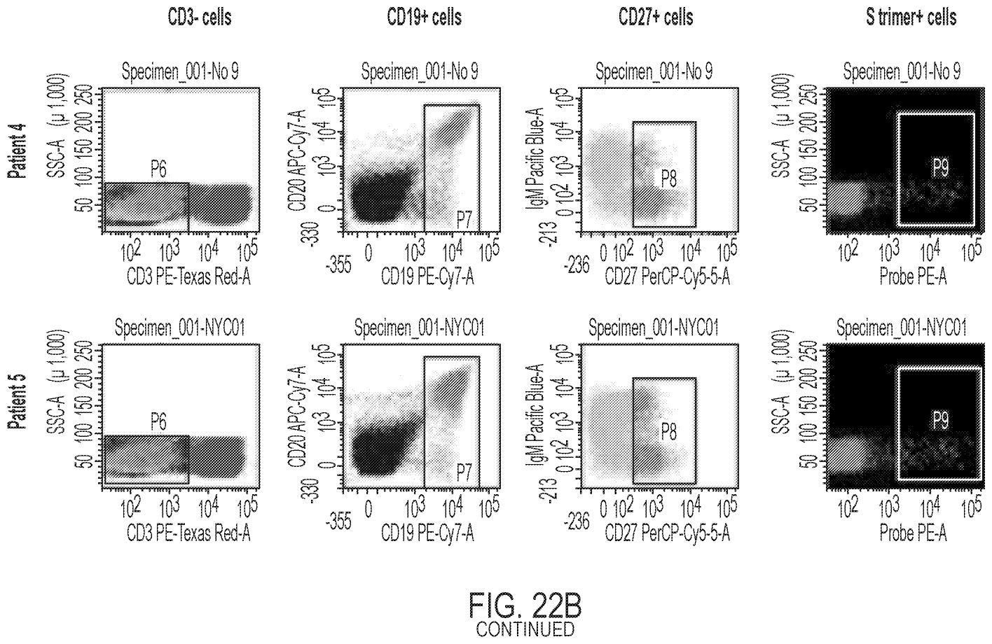

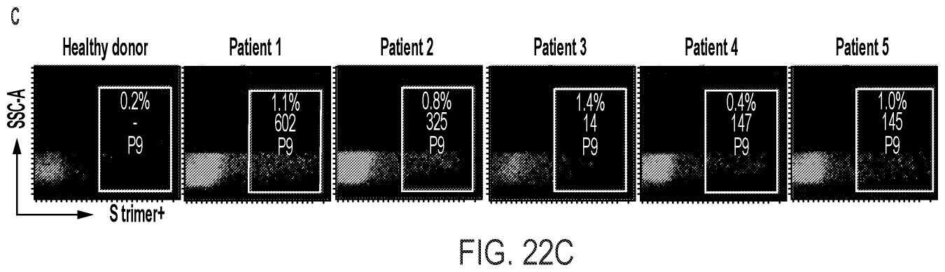

Peripheral blood mononuclear cells from each patient were put through an experimental schema (FIG. 22A) starting with cell sorting by flow cytometry. The sorting strategy focused on live memory B lymphocytes that were CD3-negative, CD19-positive, and CD27-positive (FIG. 22B). The final step focused on those cells that bound the SARS-CoV-2 spike trimer (S trimer)⁴. S trimer-positive memory B cells were enriched (0.4% to 1.4%) in the 5 patients as compared to a normal health donor (0.2%) (FIG. 22C). A total of 602, 325, 14, 147, and 145 such B cells from Patient 1, Patient 2, Patient 3, Patient 4, and Patient 5, respectively, were labelled with a unique hashtag. The cells were then placed into the 10X Chromium (10X Genomics) for single-cell 5′mRNA and V(D)J sequencing to obtain paired heavy (H) and light (L) chain sequences. A careful bioinformatic analysis was carried out on 1,145 paired sequences to downselect “high-confidence” antigen-specific mAbs. A total of 331 mAb sequences were recovered, representing 252 individual clones. Only 6 mAbs were from Patient 3, whereas 44 to 100 mAbs were identified from each of the other patients (FIG. 20). The VH and VL sequences of 252 antibodies (one per clone) were codon-optimized and synthesized, and each VH and VL gene was then cloned into an expression plasmid with corresponding constant region of H chain and L chain of human IgG1, and mAbs were expressed by co-transfection of paired full-length H chain and L chain genes into Expi293 cells. The supernatant from each transfection was collected for the screening assays and antibody purification.

Monoclonal Antibody Screening

All 252 transfection supernatants were screened for binding to S trimer and RBD by enzyme-linked immunosorbent assays (ELISAs), as well as for neutralization against SARS-CoV-2 pseudovirus and live virus. These results are graphically represented in FIGS. 15B-E and tabulated in FIG. 20. It was evident that a substantial percentage of the mAbs in the supernatants bound S trimer, and a subset of those bound RBD. Specifically, 121 supernatants were scored as positive for S trimer binding, yielding an overall hit rate of 48%. Of these, 38 were positive for RBD binding while the remaining 83 were negative. It is interesting to note that none of the 13 trimer-specific mAbs from Patient 5 recognized RBD. In the pseudovirus neutralization screen, 61 supernatants were scored as positive, indicating that half of the trimer-specific mAbs were virus neutralizing. In particular, 15 supernatants retained neutralizing activity even when diluted by 1,000-fold or more. In the screen for neutralization against SARS-CoV-2 (strain USA-WA1/2020), 41 supernatants were scored as positive, including 10 that neutralized the virus completely (+++). Overall, this screening strategy was quite effective in picking up neutralizing mAbs (vertical lines and labelled antibodies at the bottom of FIG. 15B) that were later identified as potent.

Sequence Analysis of S Trimer-Specific Monoclonal Antibodies

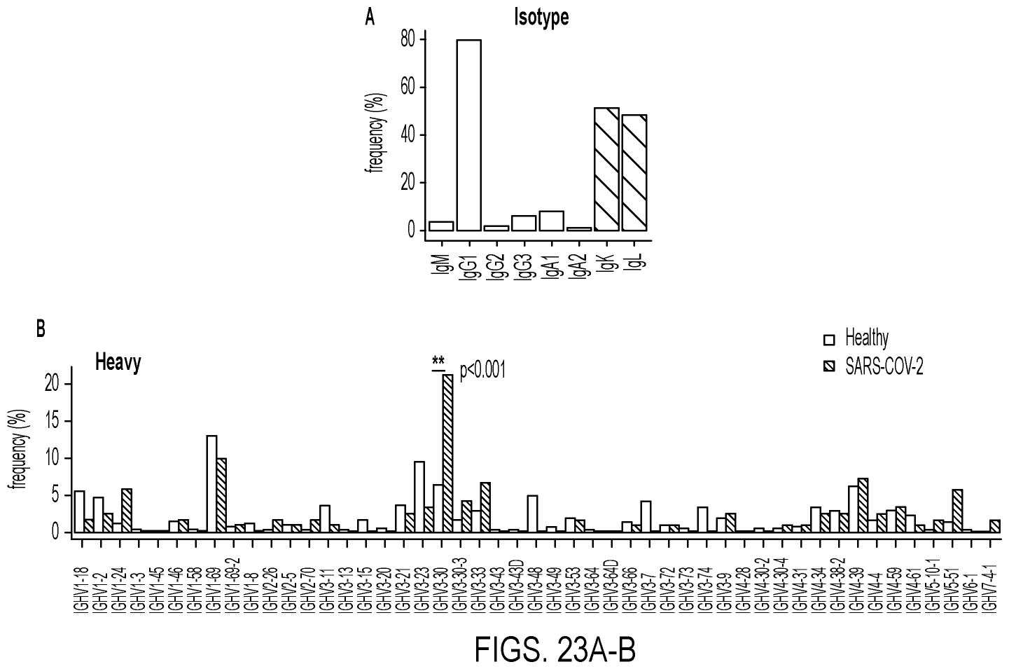

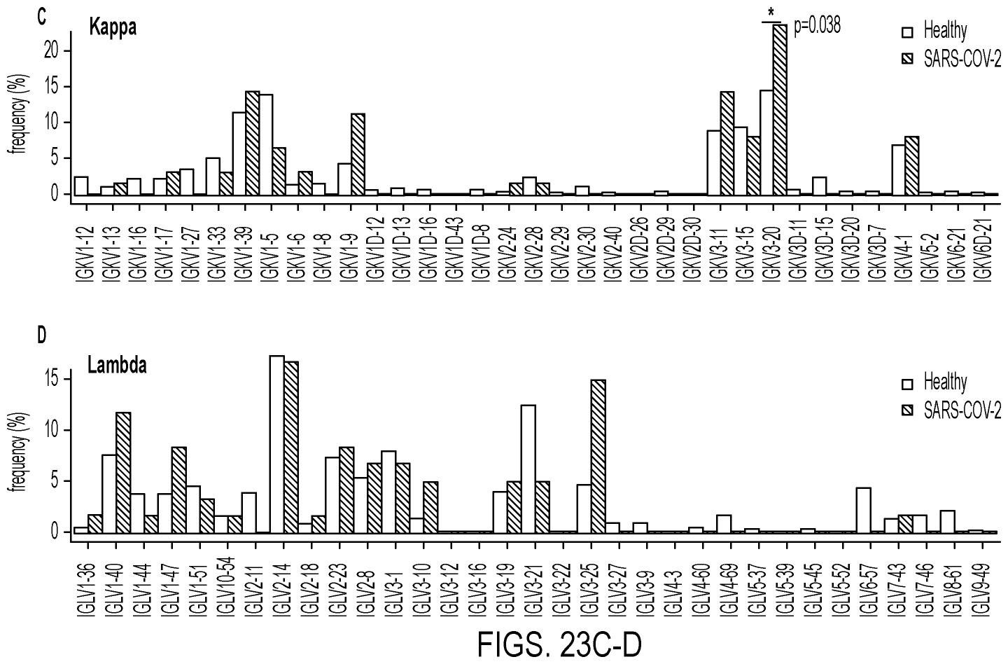

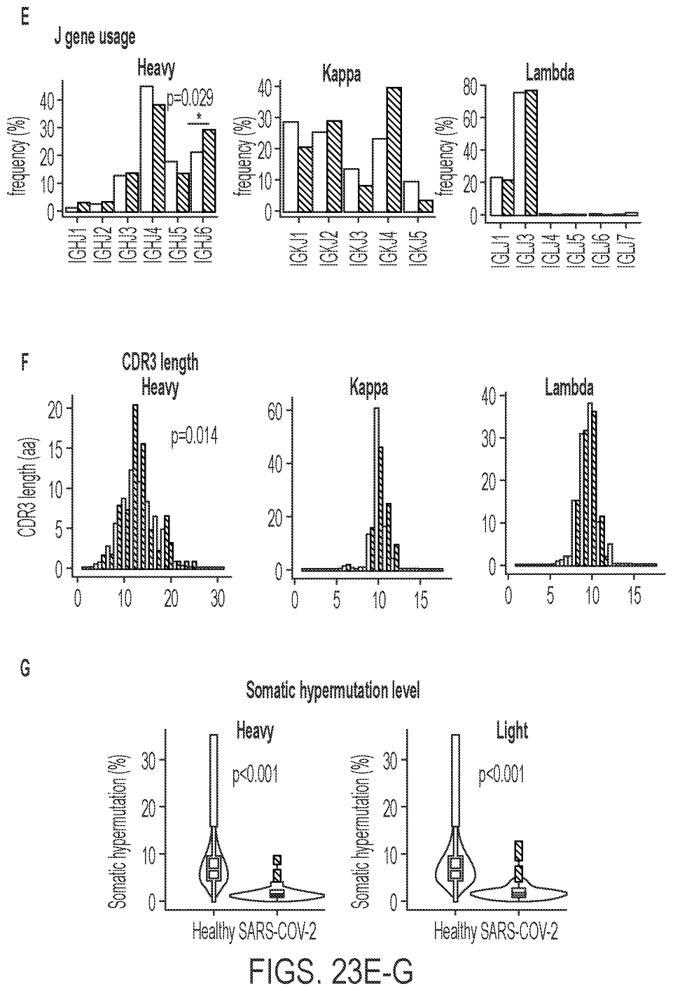

Of the 121 mAbs that bound S trimer, 88% were IgG isotype, with IgG1 being predominant (FIGS. 23A-D). Small numbers of antibodies of IgM and IgA isotypes were also found. Comparison to the IgG repertoire of three healthy human donors¹², a statistically significant over-representation of IGHV3-30, IGKV3-20, and IGHJ6 genes was observed for this collection of SARS-CoV-2 mAbs (FIG. 23E). A longer CDRH3 length was also noted (FIG. 23F). Interestingly, the average percentages of somatic hypermutation in VH and VL were 2.1 and 2.5, respectively, which were significantly lower than those found in healthy individuals (FIG. 23G) and remarkably close to germline sequences.

Antigen Binding and Virus Neutralization

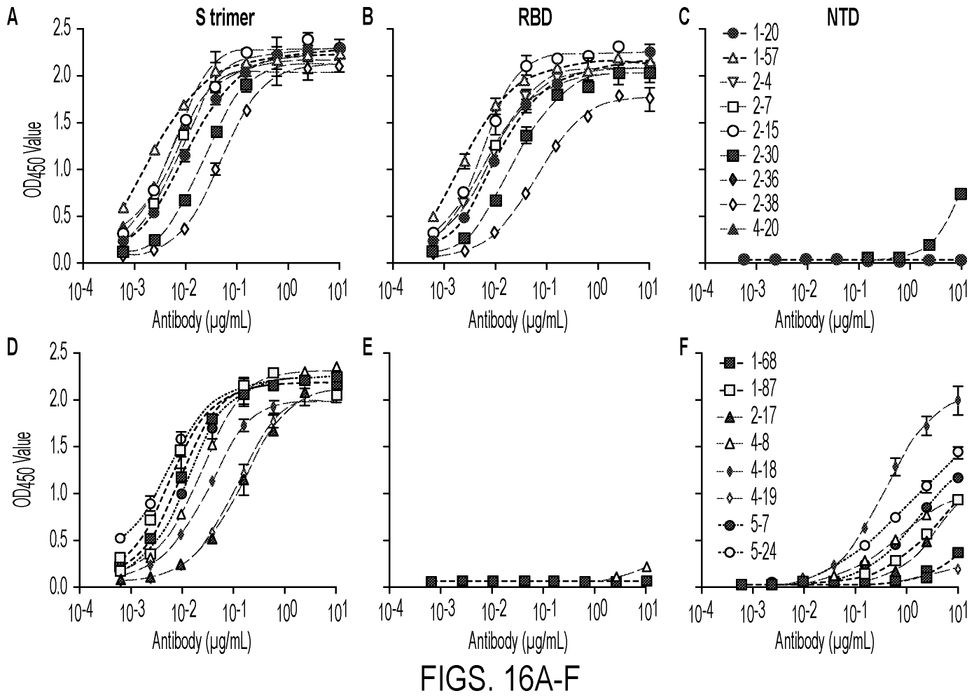

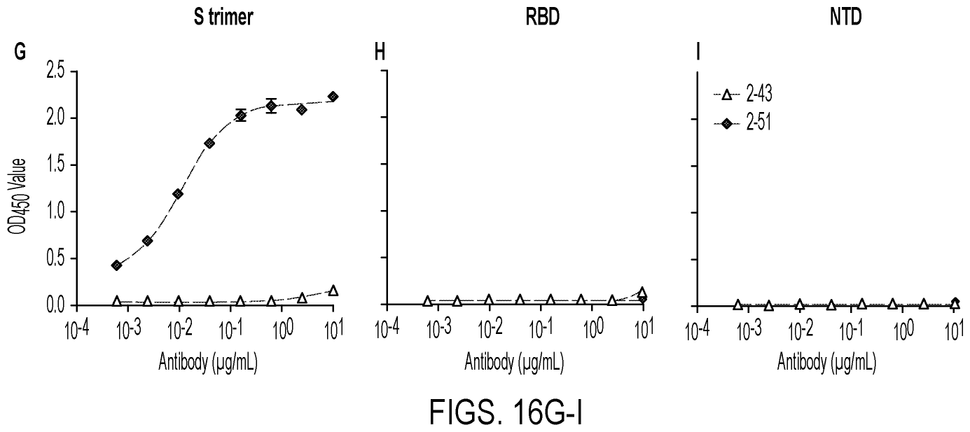

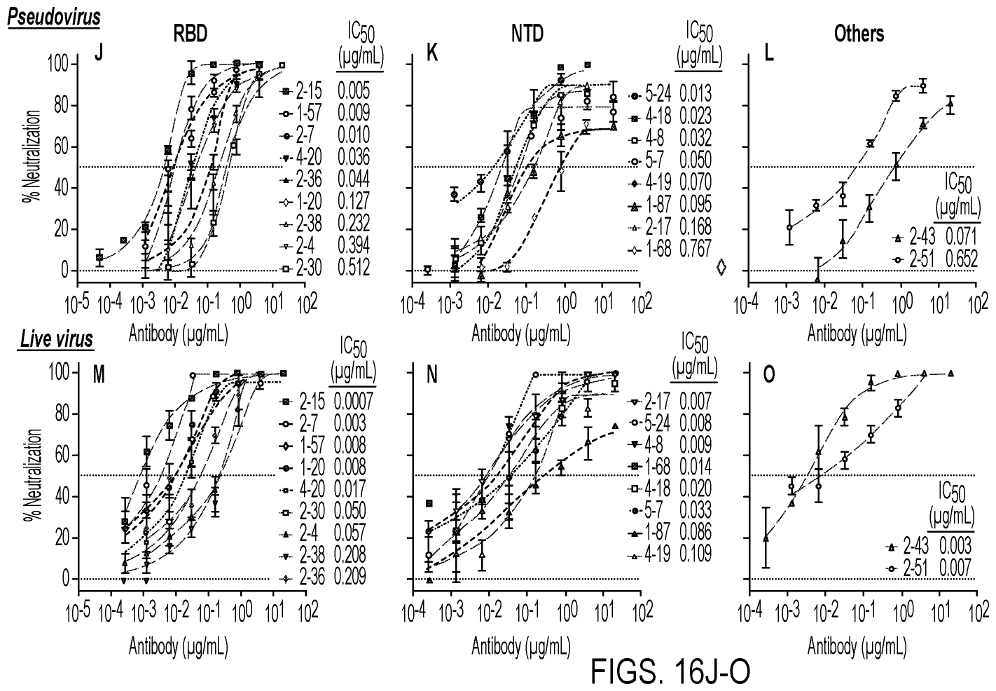

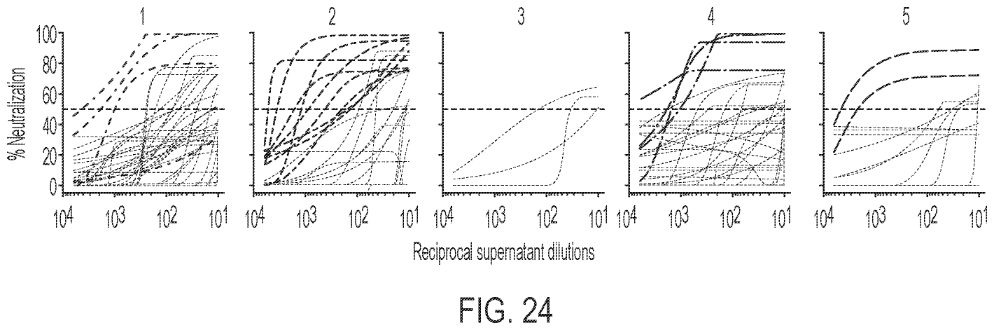

Since the screening for pseudovirus neutralization was performed quantitatively with serial dilutions of the transfection supernatants, we plotted in FIG. 24 the best-fit neutralization curves for 130 samples that were positive in at least one of the screens shown in FIG. 15B. Most were non-neutralizing or weakly neutralizing, but 18 showed evidently better potency. One additional supernatant was initially missed in the pseudovirus screen (Patient 1 in FIG. 24) but was later found to be a potent neutralizing mAb. Together, these 19 mAbs were purified from transfection supernatants and further characterized for their binding and neutralization properties. As shown in FIGS. 16A, D, and G, all but one (2-43) of the mAbs bound the S trimer by a quantitative ELISA. Using two other quantitative ELISAs, nine of the antibodies clearly bound RBD (FIGS. 16B, E, and H), with little or no binding to NTD (FIGS. 16C, F, and I). Eight antibodies bound NTD to varying degrees, with no binding to RBD. Two mAbs bound neither RBD nor NTD, and were therefore categorized as “Others”.

The pseudovirus neutralization profiles for these purified 19 mAbs are shown in the top portion of FIGS. 16J-L. The RBD-directed antibodies neutralized the pseudovirus with IC₅₀ of 0.005 to 0.512 μg/mL; the NTD-directed antibodies were slightly less potent, with IC₅₀ ranging from 0.013 to 0.767 μg/mL. A common feature of the NTD mAbs was the plateauing of virus neutralization at levels short of 100%. Two antibodies, categorized as “Others”, neutralized with IC₅₀ of 0.071 and 0.652 μg/mL, with mAb 2-51 exhibiting the plateauing effect typical of NTD antibodies. Antibody neutralization of the authentic or live SARS-CoV-2 (strain USA-WA1/2020) was carried out using Vero cells inoculated with a multiplicity of infection of 0.1. As shown in the bottom portion of FIGS. 16M-O, the RBD-directed antibodies again neutralized the virus but with IC₅₀ of 0.0007 to 0.209 μg/mL; the NTD-directed antibodies showed similar potency, with IC₅₀ ranging from 0.007 to 0.109 μg/mL. Here, the plateauing effect seen in the pseudovirus neutralization assay was less apparent. Antibodies 2-43 and 2-51 neutralized the live virus with IC₅₀ of 0.003 and 0.007 μg/mL, respectively. Overall, nine mAbs exhibited exquisite potency in neutralizing authentic SARS-CoV-2 in vitro with IC₅₀ of 0.009 μg/mL or less, including four directed to RBD (2-15, 2-7, 1-57, and 1-20), three to NTD (2-17, 5-24, and 4-8), and two to undetermined regions on the S trimer (2-43 and 2-51). It is remarkable that Patient 2 alone contributed five of the top nine SARS-CoV-2 neutralizing mAbs.

Epitope Mapping

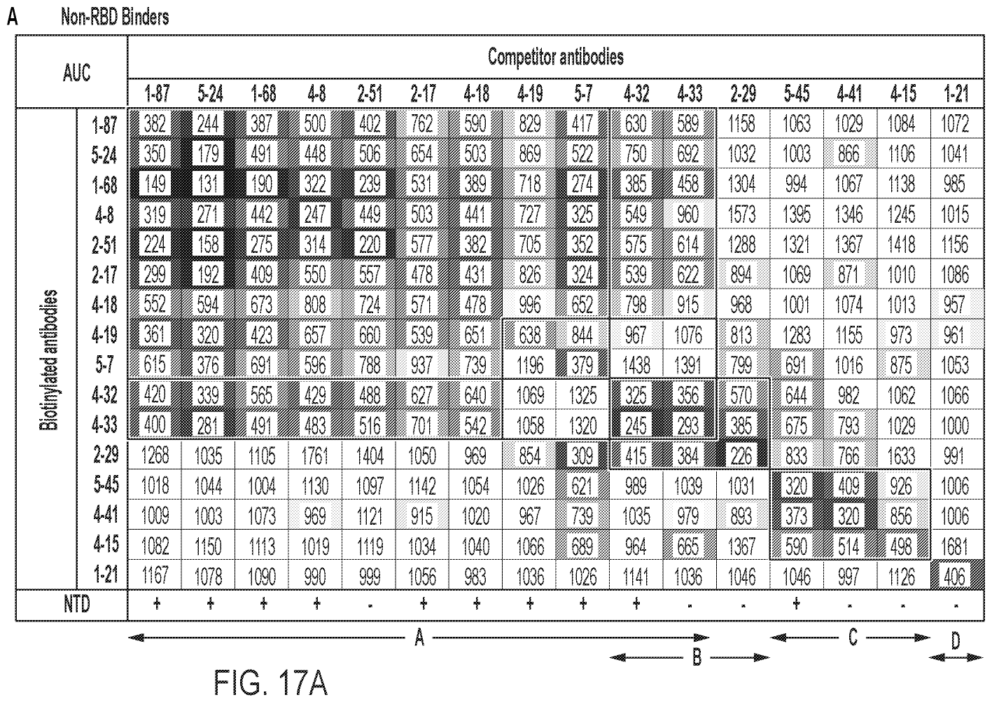

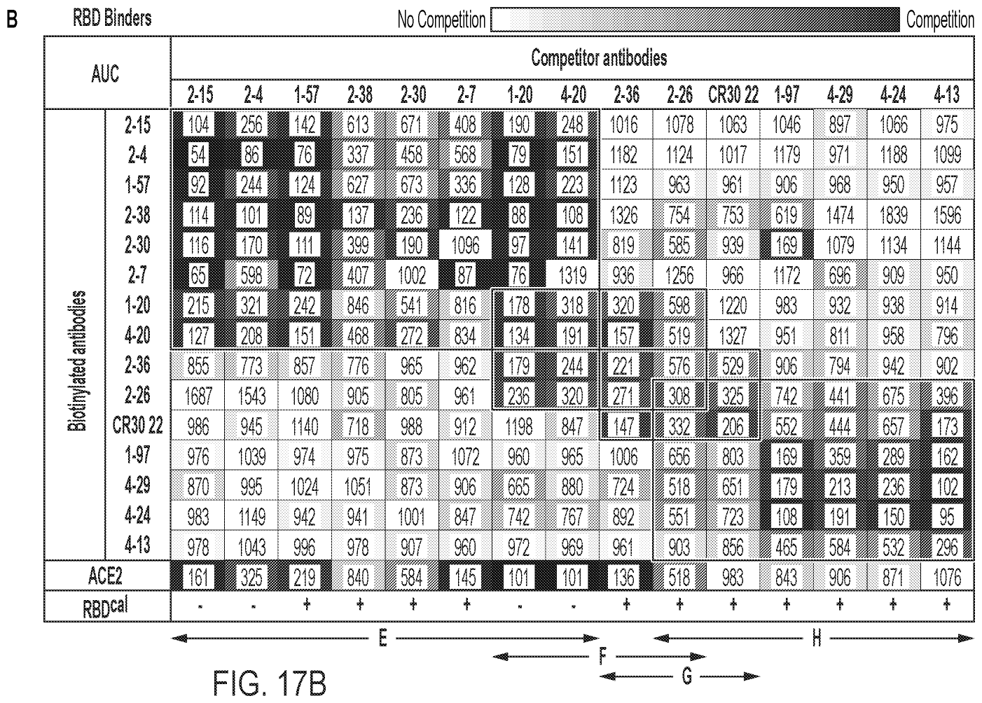

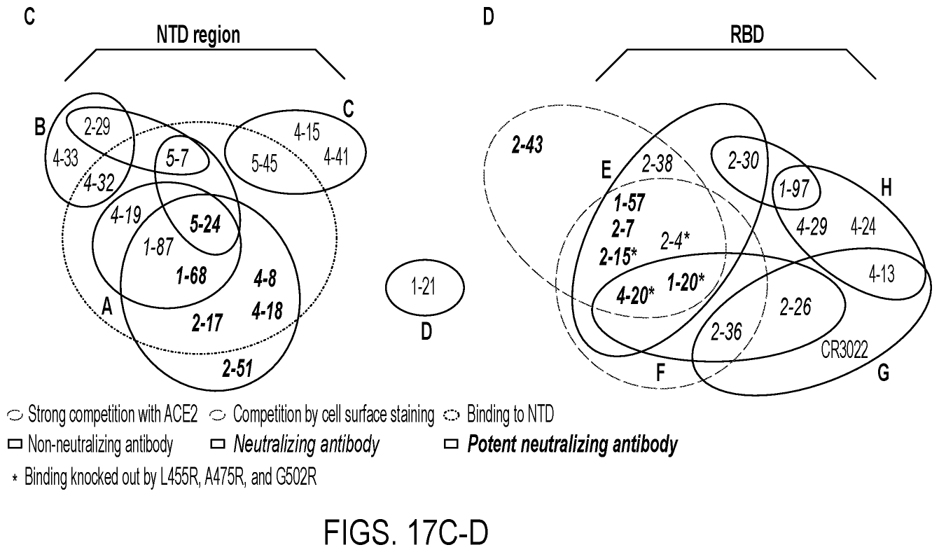

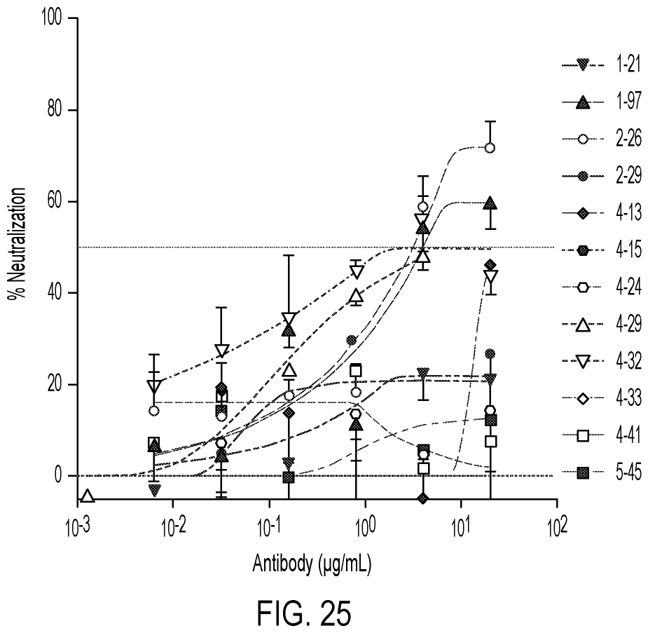

All 19 potent neutralizing mAbs (FIG. 16) were further studied in antibody competition experiments to gain insight into their epitopes. In addition, 12 mAbs that bound the S trimer strongly during the initial supernatant screen were also chosen, including 5 that bound RBD (1-97, 2-26, 4-13, 4-24, and 4-29) and 7 that did not bind RBD (1-21, 2-29, 4-15, 4-32, 4-33, 4-41, and 5-45). Four of these mAbs were weak in neutralizing SARS-CoV-2 pseudovirus, and the remaining 8 were non-neutralizing (FIG. 25). First, a total of 16 non-RBD mAbs were evaluated for competition in binding to S trimer by ELISA in a “checkerboard” experiment. The extent of the antibody competition is reflected by the intensity of the heatmap shown in FIG. 17A. There is one large cluster (A) of mAbs that competed with one another, and it partially overlaps a small cluster (B). A third cluster (C) does not overlap at all. Note that all but one of the antibodies in cluster A recognized NTD. Antibody 2-51 is clearly directed to the NTD region even though it could not bind NTD. Moreover, one mAb each from clusters B and C also recognized NTD, thereby indicating that all three clusters are within or near the NTD. One mAb, 1-21, appears to have a unique non-overlapping epitope (epitope region D).

Second, a similar “checkerboard” competition experiment was carried out by ELISA for 14 of our RBD-directed mAbs plus CR3022^{13,14}. Here, the heatmap shows that there are four epitope clusters that are serially overlapping (FIG. 17B). In FIGS. 17C-D. There is one large cluster (E) that contains mAbs largely capable of blocking ACE2 binding. Furthermore, 4 antibodies in this cluster lost binding to a mutant RBD (L455R, A475R, G502R) that could no longer bind ACE2 (our unpublished findings). Taken together, these results suggest that most of the mAbs in cluster E are directed to the ACE2-binding interface of RBD. The next cluster (F) connects to both cluster E and cluster G, the location of which is defined by its member CR3022¹⁵. Lastly, cluster G overlaps another cluster (H), which includes 1-97 that strongly inhibited the binding of 2-30 to the S trimer. This finding suggests that cluster H may be proximal to one edge of cluster E.

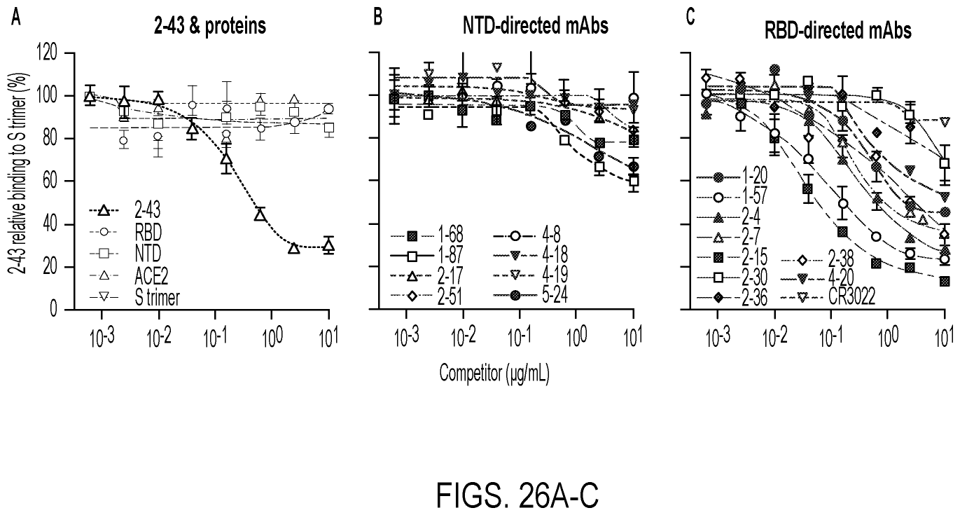

One potent neutralizing mAb, 2-43, did not bind S trimer by ELISA (FIG. 16) and thus could not be tested in the above competition experiments. However, 2-43 did strongly bind S trimer when expressed on the cell surface as determined by flow cytometry, and this binding was competed out by itself but not by RBD, NTD, ACE2, or the soluble S trimer4 (FIGS. 26A-C). NTD-directed mAbs had only a modest effect on its binding to cell-surface S trimer, but numerous RBD-directed mAbs in cluster E potently blocked the binding of 2-43, demonstrating that this antibody is likely targeting a quaternary epitope on the top of RBD that includes a portion of the interface with ACE2.

The results in FIGS. 17A-B and FIGS. 26A-C could be represented by two sets of Venn diagrams shown in FIGS. 17C-D. In the non-RBD region, the potent neutralizing mAbs reside exclusively in cluster A and bind a patch on the NTD. Weaker neutralizing mAbs recognize a region at the interface between clusters A and B. In the RBD region, the most potent neutralizing mAbs also group together within one cluster (E). Given that all block ACE2 binding, it is likely they recognize the top of RBD and neutralize the virus by competitive inhibition of receptor binding. Cluster G contains CR3022, a mAb known to be directed to an epitope on a cryptic site on the side of RBD when it is in the “up” position¹⁵. Cluster F is therefore likely situated between the top and this “cryptic” site. The Venn diagram also suggests that cluster H may occupy a different side surface near the top of RBD, perhaps in the region recognized by S3098.

Cryo-Electron Microscopy

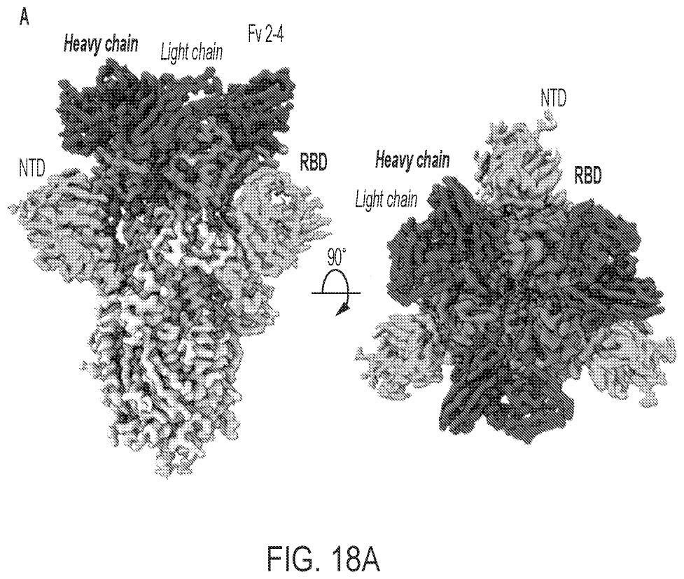

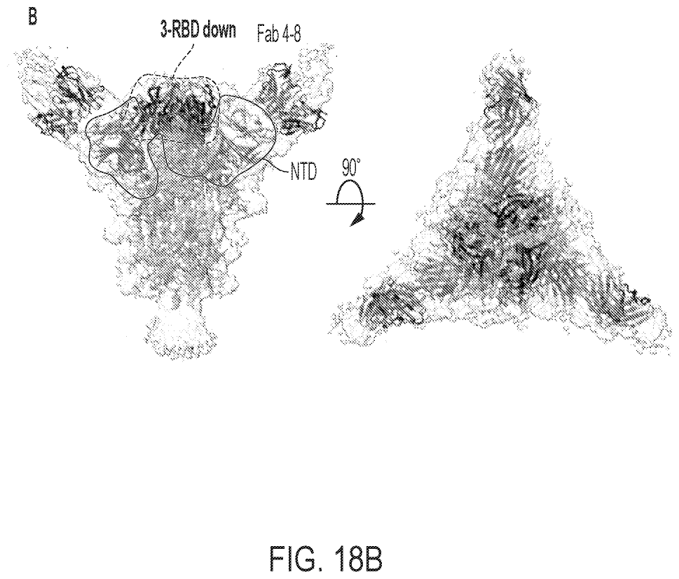

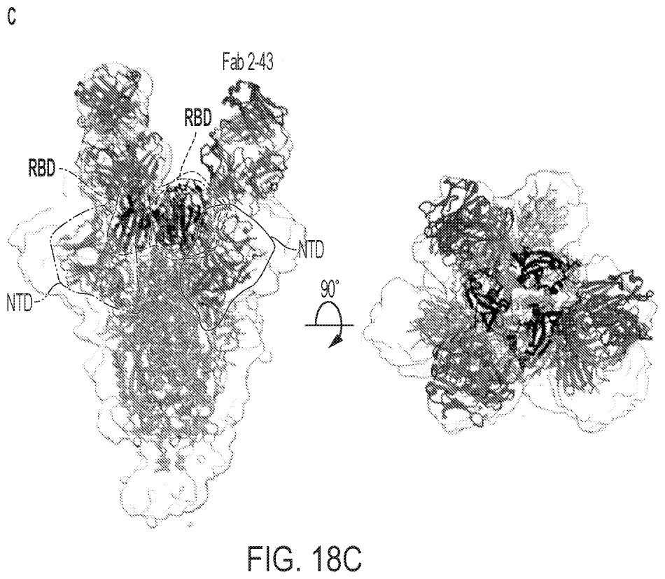

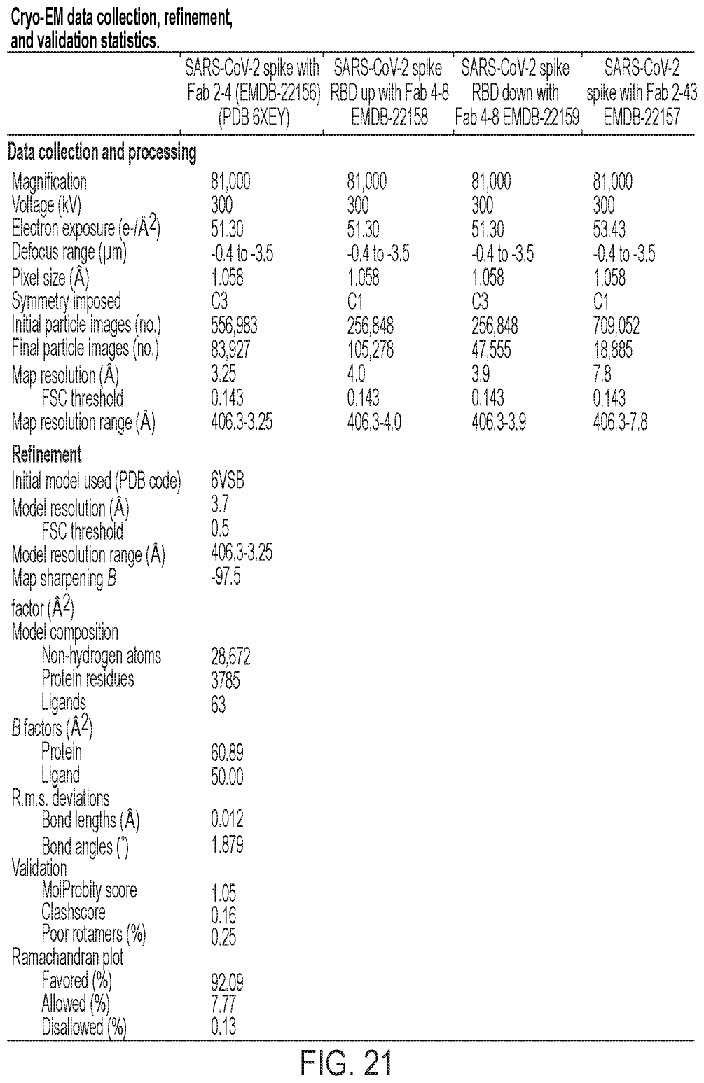

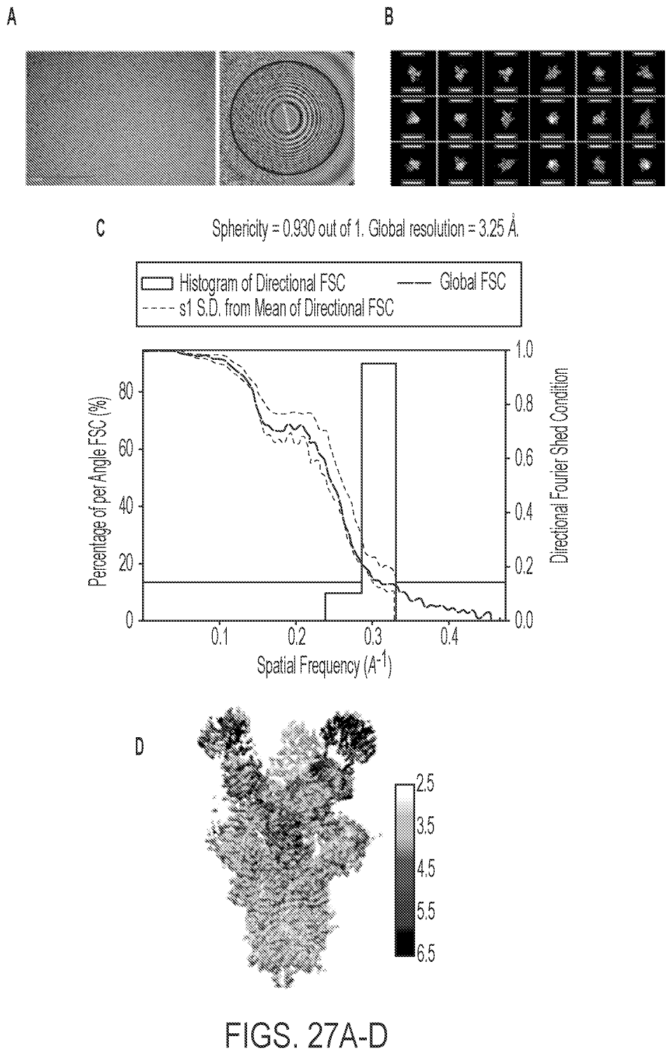

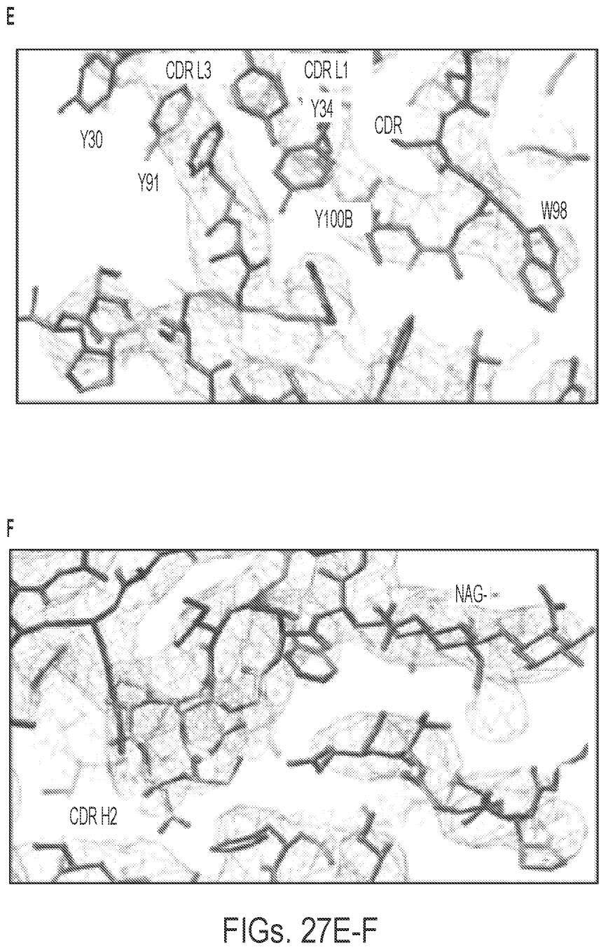

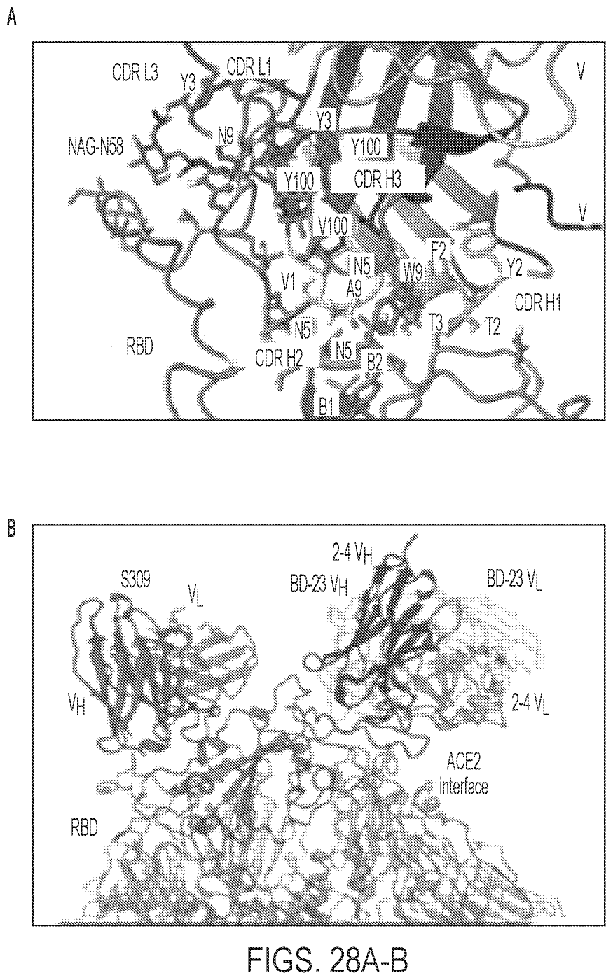

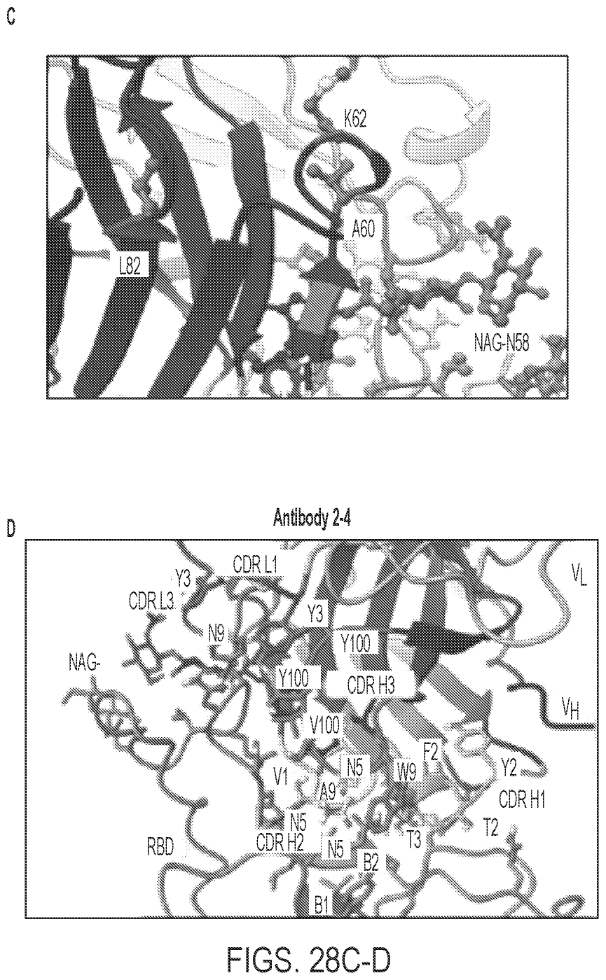

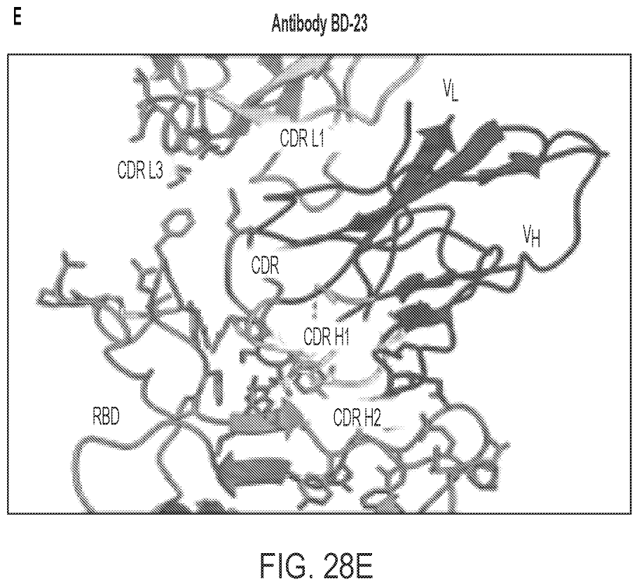

To further understand antibody recognition of the viral spike and to aid the interpretation of the mapping studies, we determined cryo-EM structures of Fabs from three mAbs in complex with the S trimer4. First, single-particle analysis of the complex with 2-4 Fab (RBD-directed) yielded maps of high quality (FIG. 18a; FIG. 21; FIGS. 27A-F), with the most abundant particle class representing a 3-Fab-per-trimer complex, refined to an overall resolution of 3.2 Å. While density for the constant portion of the Fabs was visible, it was blurred due to molecular motion, and thus only the variable domains were included in the molecular model. Fab of 2-4 bound the spike protein near the apex, with all RBDs in the “down” orientation, and the structure of the antibody-bound spike protein was highly similar to previously published unliganded spike structures in the “all-down” conformation3,4. The 2-4 epitope on RBD has a buried surface area of 751 Å2, sharing 284 Å2 with the interface of ACE2. Detailed interactions between 2-4 and RBD, along with comparative analyses, are discussed and exhibited in FIG. 28. Overall, FIG. 18A shows that neutralization of SARS-CoV-2 by mAb 2-4 likely results from locking RBD in the down conformation while also occluding access to ACE2.

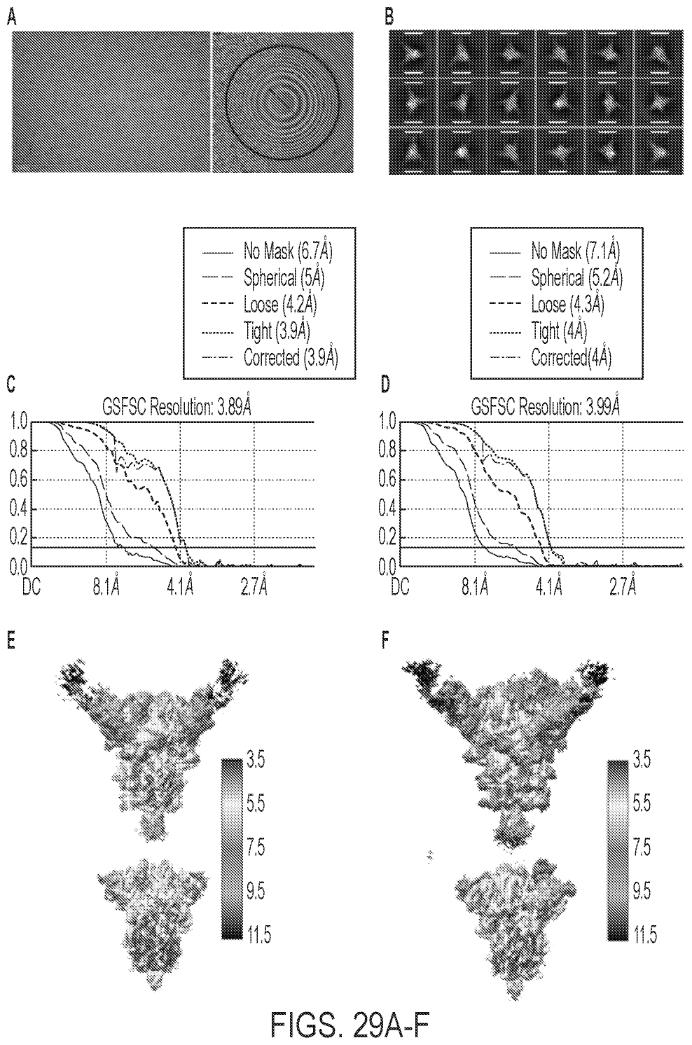

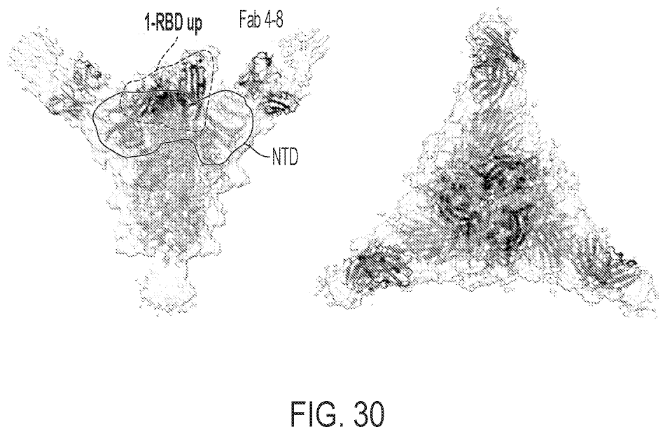

Second, we also produced 3D cryo-EM reconstructions of the Fab of 4-8 (NTD-directed) in complex with the S trimer (FIG. 21; FIG. 29). Two main particle classes were observed—one for a 3-Fab-bound complex with all RBDs “down” at 3.9 Å resolution (FIG. 18B), and another a 3-Fab-bound complex with one RBD “up” at 4.0 Å resolution (FIG. 30). However, molecular motion prevented visualization of the interaction at high resolution. Nevertheless, the density in the 4-8 map reveals the overall positions of the antibody chains targeting NTD, and helps to anchor the results of the antibody competition experiments. How such binding to the tip of NTD results in SARS-CoV-2 neutralization remains unclear.

Third, a 7.8 Å resolution reconstructions of the Fab of 2-43 in complex with the S trimer (FIG. 21; FIG. 31) revealed three bound Fabs, each targeting a quaternary epitope on the top of the spike that included the RBD of one protomer and the NTD of another (FIG. 4C), consistent with the epitope mapping results (FIG. 26 and FIG. 17B). Given these findings, the inability of 2-43 to bind S trimer by ELISA is likely an artifact of the assay format, as this mAb did bind the spike expressed on the cell surface and in the cryo-EM study. FIG. 18C suggests that 2-43 could block SARS-CoV-2 infection by occluding the site necessary for ACE2 binding.

Armed with these three cryo-EM reconstructions, we used the Venn diagrams from FIG. 17B to map the epitopes of many of our SARS-CoV-2 neutralizing mAbs onto the surface of the spike (FIG. 18D). This is obviously a rough approximation since antibody footprints are much larger than the area occupied by the mAb number. However, the spatial relationship of the antibody epitopes should be reasonably represented by FIG. 18D.

### DISCUSSION

We have discovered a collection of SARS-CoV-2-neutralizing mAbs that are not only potent but also diverse. Nine of these antibodies can neutralize the authentic virus in vitro at concentrations of 9 ng/mL of less (FIG. 16B), including 4 directed to RBD, 3 directed to NTD, and 2 to quaternary epitopes nearby. Surprisingly, many of the these mAbs have V(D)J sequences close to germline sequences, without extensive somatic hypermutations (FIG. 23E), a finding that bodes well for vaccine development. Our most potent RBD-specific mAbs (e.g., 2-15, 2-7, 1-57, and 1-20) compare favorably with such antibodies recently reported^{7,8,10,16-20} including those with high potency^{9,11,21,22}. It appears from the epitope mapping studies that mAbs directed to the top of RBD strongly compete with ACE2 binding and potently neutralize the virus, whereas those directed to the side surfaces of RBD do not compete with ACE2 and neutralize less potently (FIGS. 3B and 4D). Our collection of non-RBD neutralizing mAbs is unprecedented in that such antibodies only have been sporadically reported and only with substantially lower potencies²²⁻²⁴. The most potent of these mAbs are directed to (e.g., 2-17, 5-24, and 4-8) or overlapping with (2-51) a patch on the NTD (FIGS. 3B and 4D). It is unclear how NTD-directed mAbs block SARS-CoV-2 infection and why their neutralization profiles are different from those of RBD-directed antibodies (FIG. 16B). Nevertheless, vaccine strategies that do not include NTD will be unable to induce an important class of virus-neutralizing antibodies.

The isolation of two mAbs (2-43 and 2-51) directed to epitopes that do not map to RBD and NTD is also unprecedented. Cryo-EM of 2-43 bound to the S trimer has confirmed its epitope as quaternary in nature, crossing from the top of RBD to the top of an adjacent NTD (FIG. 18C). It will be equally informative to understand the epitope of 2-51 as well. In this study, we also show the first evidence by cryo-EM for a neutralizing mAb (4-8) bound to the NTD of the viral spike (FIG. 18B), as well as another high-resolution structure of a mAb (2-4) bound to RBD (FIG. 18A). Collectively, these findings will contribute to the understanding of how antibodies bind and neutralize SARS-CoV-2.

The potency and diversity of our SARS-CoV-2-neutralizing mAbs are likely attributable to patient selection. Previously, we observed that infected individuals with severe disease developed a more robust virus-neutralizing antibody response²⁵. If Patient 2 had not been included, five of the top neutralizing mAbs would be lost. The diversity of our antibodies is also attributable, in part, to the choice of using the S trimer to sort from memory B cells, while most groups focused on the use of RBD^{7,9-11,16-19,21}. The characterization of this diverse collection of mAbs has allowed us to observe that all potent SARS-CoV-2-neutralizing antibodies described to date are directed to the top of the viral spike. RBD and NTD are, no doubt, quite immunogenic. Neutralizing antibodies to the stem region of the S trimer remain to be discovered. In conclusion, we believe several of our monoclonal antibodies with exquisite virus-neutralizing activity are promising candidates for development as modalities to treat or prevent SARS-CoV-2 infection.

Methods

Expression and Purification of SARS-CoV-2 Proteins

The mammalian expression vector that encodes the ectodomain of the SARS-CoV-2 S trimer and the vector encoding RBD fused with SD1 at the N-terminus and an HRV-3C protease cleavage site followed by a mFc tag and an 8xHis tag at the C-terminus were kindly provided by Jason McLellan⁴. SARS-CoV-2 NTD (aa1-290) with an HRV-3C protease cleavage site, a mFc tag, and an 8xHis tag at the C-terminus was also cloned into mammalian expression vector pCAGGS. Each expression vector was transiently transfected into Expi293 cells using 1 mg/mL of polyethylenimine (Polysciences). Five days post transfection, the S trimer was purified using Strep-Tactin XT Resin (Zymo Research), and the RBD-mFc and NTD-mFc were purified using protein A agarose (ThermoFisher Scientific). In order to obtain RBD-SD1 and NTD, the mFc and 8xHis tags at the C-terminus were removed by HRV-3C protease (Millipore-Sigma) and then purified using Ni-NTA resin (Invitrogen) followed by protein A agarose.

Sorting for S Trimer-Specific B cells and Single-Cell BCR Sequencing

Peripheral blood mononuclear cells from five patients and one healthy donor were stained with LIVE/DEAD™ Fixable Yellow Dead Cell Stain Kit (Invitrogen) at ambient temperature for 20 mins, followed by washing with RPMI-1640 complete medium and incubation with 10 μg/mL of S trimer at 4° C. for 45 mins. Afterwards, the cells were washed again and incubated with a cocktail of flow cytometry and hashtag antibodies, containing CD3 PE-CF594 (BD Biosciences), CD19 PE-Cy7 (Biolegend), CD20 APC-Cy7 (Biolegend), IgM V450 (BD Biosciences), CD27 PerCP-Cy5.5 (BD Biosciences), anti-His PE (Biolegend), and human Hashtag 3 (Biolegend) at 4° C. for 1 hr. Stained cells were then washed, resuspended in RPMI-1640 complete medium and sorted for S trimer-specific memory B cells (CD3-CD19+CD27+S trimer+ live single lymphocytes). The sorted cells were mixed with mononuclear cells from the same donor, labeled with Hashtag 1, and loaded into the 10X Chromium chip of the 5′ Single Cell Immune Profiling Assay (10X Genomics) at the Columbia University Human Immune Monitoring Core (HIMC; RRID:SCR 016740). The library preparation and quality control were performed according to manufacturer's protocol and sequenced on a NextSeq 500 sequencer (Illumina).

Identification of S Trimer-Specific Antibody Transcripts

For each sample, full-length antibody transcripts were assembled using the VDJ module in Cell Ranger (version 3.1.0, 10X Genomics) with default parameters and the GRCh38 genome as reference. To identify cells from the antigen sort, we first used the count module in Cell Ranger to calculate copies of all hashtags in each cell from the Illumina NGS raw reads. High confidence antigen-specific cells were identified as follows. Briefly, based on the copy numbers of the hashtags observed, a cell must contain more than 100 copies of the antigen sort-specific hashtag to qualify as an antigen-specific cell. Because hashtags can fall off from cells and bind to cells from a different population in the sample mixture, each cell usually has both sorted and spiked-in-specific hashtags. To enrich for true antigen-specific cells, the copy number of the specific hashtag has to be at least 1.5×higher than that of the non-specific hashtag. Low quality cells were identified and removed using the cell-calling algorithm in Cell Ranger. Cells that do not have productive H and L chain pairs were excluded. If a cell contains more than two H or/and L chain transcripts, the transcripts with less than 3 unique molecular identifiers were removed. Cells with identical H and L chain sequences, which may have resulted from mRNA leakage, were merged into one cell. Additional filters were applied to remove low quality cells and/or transcripts in the antibody gene annotation process.

Antibody Transcript Annotation and Selection Criteria

Antigen-specific antibody transcripts were processed using our bioinformatics pipeline SONAR for quality control and annotation²⁶. Briefly, V(D)J genes were assigned for each transcript using BLAST²⁷ with customized parameters against a germline gene database obtained from the international ImMunoGeneTics information system (IMGT) database^{26,28}. Based on BLAST alignments of V and J regions, CDR3 was identified using the conserved second cysteine in the V region and WGXG (H chain) or FGXG (L chain) motifs in the J region (X represents any amino acid). For H chain transcripts, the constant domain 1 (CH1) sequences were used to assign isotype using BLAST with default parameters against a database of human CH1 genes obtained from IMGT. A BLAST E-value threshold of 1E-6 was used to find significant isotype assignments, and the CH1 allele with the lowest E-value was used. Sequences other than the V(D)J region were removed and transcripts containing incomplete V(D)J or/and frame shift were excluded. We then aligned each of the remaining transcripts to the assigned germline V gene using CLUSTALO²⁹ and calculated the somatic hypermutation level.

To select representative antibodies for functional characterization, we first clustered all antibodies using USEARCH³⁰ with the following criteria: identical heavy chain V and J gene assignments, the same length of CDRH3, and CDRH3 identity higher than 0.9. For each cluster, cells with the same light chain V and J gene assignments were grouped into a clone. All clone assignments were manually checked. We then calculated the clonal size for each clone, and one H and L chain pair per clone was chosen for antibody synthesis. For clones with multiple members, the member with the highest somatic hypermutation level was chosen for synthesis. For cells having multiple high quality H or L chains, which may be from doublets, we synthesized all H and L chain combinations.

Analysis of S Trimer-Specific Antibody Repertoire

Because 88% of the S trimer-specific antibodies were IgG isotype, we therefore compared the repertoire features to IgG repertoires from three healthy donors³¹ (17,243 H chains, 27,575 kappa L chains, 20,889 lambda L chains). The repertoire data from the three healthy donors were combined and annotated using SONAR with the same process as above.

Antibody Expression and Purification

For each antibody, variable genes were optimized for human cell expression and synthesized by GenScript. VH and VL were inserted separately into plasmids (gWiz or pcDNA3.4) that encode the constant region for H chain and L chain. Monoclonal antibodies were expressed in Expi293 (ThermoFisher, A14527) by co-transfection of H chain and L chain expressing plasmids using polyethylenimine and culture in 37° C. shaker at 125 RPM and 8% CO2. On day 3 post transfection, 400 μL of supernatant were collected for screening for binding to S trimer and RBD by ELISA, and for neutralization of SARS-CoV-2 pseudovirus and authentic virus. Supernatants were also collected on day 5 for antibody purification by rProtein A Sepharose (GE, 17-1279-01) affinity chromatography.

Production of Pseudoviruses

Recombinant Indiana VSV (rVSV) expressing SARS-CoV-2 spike was generated as previously described32,33. HEK293T cells were grown to 80% confluency before transfection with pCMV3—SARS-CoV-2-spike (Sino Biological) using FuGENE 6 (Promega). Cells were cultured overnight at 37° C. with 5% CO2. The next day, medium was removed and VSV-G pseudotyped ΔG-luciferase (G*ΔG-luciferase, Kerafast) was used to infect the cells in DMEM at a MOI of 3 for 1 hr before washing the cells with 1X DPBS three times. DMEM supplemented with 2% fetal bovine serum and 100 I.U./mL of penicillin and 100 μg/mL of streptomycin were added to the inoculated cells, which were cultured overnight as described above. The supernatant was harvested the following day and clarified by centrifugation at 300 g for 10 mins before aliquoting and storing at −80° C.

Pseudovirus Neutralization

Neutralization assays were performed by incubating pseudoviruses with serial dilutions of heat inactivated plasma together with supernatant or purified antibodies, and scored by the reduction in luciferase gene expression. In brief, Vero E6 cells (ATCC) were seeded in a 96-well plate at a concentration of 2×104 cells per well. Pseudoviruses were incubated the next day with serial dilutions of the test samples in duplicate or triplicate for 30 mins at 37° C. The mixture was added to cultured cells and incubated for an additional 24 hrs. The luminescence was measured by Britelite plus Reporter Gene Assay System (PerkinElmer). IC₅₀ was defined as the dilution at which the relative light units were reduced by 50% compared with the virus control wells (virus+cells) after subtraction of the background in the control groups with cells only. The IC50 values were calculated using non-linear regression in GraphPad Prism 8.0.

Authentic SARS-CoV-2 Neutralization

Supernatants containing expressed mAbs were diluted 1:10 and 1:50 in EMEM with 7.5% inactivated fetal calf serum and incubated with authentic SARS-CoV-2 (strain USA-WA1/2020; MOI 0.1) for 1 hr at 37° C. Post-incubation, the mixture was transferred onto a monolayer of Vero E6 cells that was cultured overnight. After incubation of the cells with the mixture for 70 hrs at 37° C., cytopathic effects (CPE) caused by the infection were scored for each well from 0 to 4 to indicate the degree of virus inhibition. Semi-quantitative representation of the inhibition for each antibody-containing supernatant at a dilution of 1:50 is shown in the lowest panel of FIG. 15B with neutralization levels ranging from (−) for none to (+++) for complete neutralization.

An end-point dilution assay in a 96-well plate format was performed to measure the neutralization activity of select purified mAbs. In brief, each antibody was serially diluted (5-fold dilutions) starting at 20 μg/mL. Triplicates of each mAb dilution were incubated with SARS-CoV-2 at aMOI of 0.1 in EMEM with 7.5% inactivated fetal calf serum for 1 hr at 37° C. Post incubation, the virus-antibody mixture was transferred onto a monolayer of Vero-E6 cells grown overnight. The cells were incubated with the mixture for 70 hrs. CPE were visually scored for each well in a blinded fashion by two independent observers. The results were then converted into percentage neutralization at a given mAb concentration, and the averages ±SEM were plotted using a five-parameter dose-response curve in GraphPad Prism 8.0.

Epitope Mapping by ELISA

50 ng/well of S trimer, 50 ng/well of RBD, and 100 ng/well of NTD were coated on ELISA plates at 4° C. overnight. The ELISA plates were then blocked with 300 μL of blocking buffer (1% BSA and 10% bovine calf serum (BCS) (Sigma) in PBS at 37° C. for 2 hrs. Afterwards, supernatants from the antibody transfection or purified antibodies were serially diluted using dilution buffer (1% BSA and 20% BCS in PBS), incubated at 37° C. for 1 hr. Next, 100 μL of 10,000-fold diluted Peroxidase AffiniPure goat anti-human IgG (H+L) antibody (Jackson ImmunoResearch) were added into each well and incubated for 1 hr at 37° C. The plates were washed between each step with PBST (0.5% Tween-20 in PBS). Finally, the TMB substrate (Sigma) was added and incubated before the reaction was stopped using 1M sulfuric acid. Absorbance was measured at 450 nm.

For the competition ELISA, purified mAbs were biotin-labeled using One-Step Antibody Biotinylation Kit (Miltenyi Biotec) following manufacturer recommendations and purified using 40K MWCO Desalting Column (ThermoFisher Scientific). 50 μL of serially diluted competitor antibodies were added into S trimer-precoated ELISA plates, followed by 50 μL of biotinylated antibodies at a concentration that achieves an OD450 reading of 1.5 in the absence of competitor antibodies. Plates were incubated at 37° C. for 1 hr, and 100 μL of 500-fold diluted Avidin-HRP (ThermoFisher Scientific) were added into each well and incubated for another 1 hr at 37° C. The plates were washed by PBST between each of the previous steps. The plates were developed afterwards with TMB and absorbance was read at 450 nm after the reaction was stopped.

For the ACE2 competition ELISA, 100 ng of ACE2 protein (Abcam) was immobilized on the plates at 4° C. overnight. The unbound ACE2 was washed away by PBST and then the plates were blocked. After washing, 100 ng of S trimer in 50 μL of dilution buffer was added into each well, followed by adding another 50 μL of serially diluted competitor antibodies and then incubating the plates at 37° C. for 1 hr. The ELISA plates were washed 4 times by PBST and then 100 μL of 2000-fold diluted anti-strep-HRP (Millipore Sigma) were added into each well for another 1 hr at 37° C. The plates were then washed, developed with TMB, and absorbance was read at 450 nm after the reaction was stopped.

For all the competition ELISA experiments, the relative binding of biotinylated antibodies or ACE2 to the S trimer in the presence of competitors was normalized by comparing to competitor-free controls. Relative binding curve and the area under curve (AUC) were generated by fitting the non-linear five-parameter dose-response curve in GraphPad Prism 8.0.

Cell-Surface Competition Binding Assay

Expi293 cells were co-transfected with vectors encoding pRRL-cPPT-PGK-GFP (Addgene) and pCMV3-SARS-CoV-2 (2019-nCoV) Spike (Sino Biological) at a ratio of 1:1. Two days after transfection, cells were incubated with a mixture of biotinylated mAb 2-43 (0.25 μg/mL) and serially diluted competitor antibodies at 4° C. for 1 hr. Then 100 μL of diluted APC-streptavidin (Biolegend) were added to the cells and incubated at 4° C. for 45 mins. Cells were washed 3 times with FACS buffer before each step. Finally, cells were resuspended and 2-43 binding to cell-surface S trimer was quantified on LSRII flow cytometer (BD Biosciences). The mean fluorescence intensity of APC in GFP-positive cells was analyzed using FlowJo and the relative binding of 2-43 to S trimer in the presence of competitors was calculated as the percentage of the mean fluorescence intensity compared to that of the competitor-free controls.

Cryo-EM Data Collection and Processing

SARS-CoV-2 S trimer at a final concentration of 2 mg/ml was incubated with 6-fold molar excess per spike monomer of the antibody Fab fragments for 30 mins in 10 mM Tris-HCl, 150 mM NaCl, and 0.005% n-Dodecyl-β-D-maltoside (DDM). 2 μL of sample were incubated on C-flat 1.2/1.3 carbon grids for 30 secs and vitrified using a Leica EM GP Plunge Freezer. Data were collected on a Titan Krios electron microscope operating at 300 kV equipped with a Gatan K3 direct detector and energy filter using the Leginon software package34. A total electron fluence of 51.3 e/Å2 was fractionated over 40 frames, with a total exposure time of 2 secs. A magnification of 81,000×resulted in a pixel size of 1.058 Å, and a defocus range of −0.4 to −3.5 μm was used. All processing was done using cryoSPARC v2.14.235. Raw movies were aligned and dose-weighted using patch motion correction, and the CTF was estimated using patch CTF estimation. A small subset of approximately 200 micrographs were picked using blob picker, followed by 2D classification and manual curation of particle picks, and used to train a Topaz neural network³⁶. This network was then used to pick particles from the remaining micrographs, which were extracted with a box size of 384 pixels. For the Fab 2-4 dataset, 2D classification followed by ab initio modelling and 3D heterogeneous refinement revealed 83,927 particles with three 2-4 Fabs bound, one to each RBD. Our construction of these particles using Non-Uniform Refinement with imposed C3 symmetry resulted in a 3.6 Å map, as determined by the gold standard FSC. Given the relatively low resolution of the RBD-Fab interface, masked local refinement was used to obtain a 3.5 Å map with significantly improved density. A masked local refinement of the remainder of the S timer resulted in a 3.5 Å reconstruction. These two local refinements were aligned and combined using the vop maximum function in UCSF Chimera³⁷. This was repeated for the half maps, which were used, along with the refinement mask from the global Non-Uniform refinement, to calculate the 3DFSC38 and obtain an estimated resolution of 3.2 Å. All maps have been submitted to the EMDB with the ID EMD-22156. For the Fab 4-8 dataset, image preprocessing and particle picking was performed as above. 2D classification, ab initio modelling, and 3D heterogeneous classification revealed 47,555 particles with 3 Fabs bound, one to each NTD and with all 3 RBDs in the down conformation. While this particle stack was refined to 3.9 Å using Non-Uniform refinement with imposed C3 symmetry, significant molecular motion prevented the visualization of the Fab epitope at high resolution (EMD-22159). In addition, 105,278 particles were shown to have 3 Fabs bound, but with 1 RBD in the up conformation. These particles were refined to 4.0 Å using Non-Uniform refinement with Cl symmetry (EMD-22158), and suffered from the same conformational flexibility as the all-RBD-down particles. This flexibility was visualized using 3D variability analysis in cryoSPARC. For the Fab 2-43 dataset, which was collected at an electron fluence of 53.4 e/Å2, image preprocessing and particle picking was performed as above, save that motion correction was performed using MotionCor239. 2D classification, ab initio modelling, and 3D heterogeneous classification revealed 18,884 particles with 3 Fabs bound, which was refined to 7.8 Å resolution 601 (EMD-22157).

Cryo-EM Model Fitting

An initial homology model of the 2-4 Fab was built using Schrodinger Release 2020-2: BioLuminate⁴⁰. The RBD was initially modeled using the coordinates from PDB ID 6W41. The remainder of the S timer was modeled using the coordinates from PDB ID 6VSB. These models were docked into the consensus map using Chimera. The model was then fitted interactively using ISOLDE 1.0b541 and COOT 0.8.9.242, and using real space refinement in Phenix 1.1843. In cases where side chains were not visible in the experimental data, they were truncated to alanine. Validation was performed using Molprobity⁴⁴ and EMRinger⁴⁵. The model was submitted to the PDB with the ID 6XEY. Figures were prepared using ChimeraX⁴⁶.

### REFERENCES FOR EXAMPLE 10

1. Zhou, P. et al. A pneumonia outbreak associated with a new coronavirus of probable bat 277 origin. Nature 579, 270-273, doi:10.1038/s41586-020-2012-7 (2020).
2. Wang, C., Horby, P. W., Hayden, F. G. & Gao, G. F. A novel coronavirus outbreak of global 279 health concern. Lancet 395, 470-473, doi:10.1016/S0140-6736(20)30185-9 (2020).
3. Walls, A. C. et al. Structure, Function, and Antigenicity of the SARS-CoV-2 Spike 281 Glycoprotein. Cell 181, 281-292 e286, doi:10.1016/j.cell.2020.02.058 (2020).
4. Wrapp, D. et al. Cryo-EM structure of the 2019-nCoV spike in the prefusion conformation. 283 Science 367, 1260-1263, doi:10.1126/science.abb2507 (2020).
5. Hoffmann, M. et al. SARS-CoV-2 Cell Entry Depends on ACE2 and TMPRSS2 and Is Blocked by a Clinically Proven Protease Inhibitor. Cell 181, 271-280 e278, doi:10.1016/j.cell.2020.02.052 (2020).
6. Wang, Q. et al. Structural and Functional Basis of SARS-CoV-2 Entry by Using Human ACE2. Cell 181, 894-904 e899, doi:10.1016/j.cell.2020.03.045 (2020).
7. Ju, B. et al. Human neutralizing antibodies elicited by SARS-CoV-2 infection. Nature, doi:10.1038/s41586-020-2380-z (2020).
8. Pinto, D. et al. Cross-neutralization of SARS-CoV-2 by a human monoclonal SARS-CoV antibody. Nature, doi:10.1038/s41586-020-2349-y (2020).
9. Cao, Y. et al. Potent neutralizing antibodies against SARS-CoV-2 identified by high-throughput single-cell sequencing of convalescent patients' B cells. Cell, doi:10.1016/j.cell.2020.05.025 (2020).
10. Wu, Y. et al. A noncompeting pair of human neutralizing antibodies block COVID-19 virus binding to its receptor ACE2. Science 368, 1274-1278, doi:10.1126/science.abc2241 (2020).
11. Hansen, J. et al. High-Throughput Effort Using Both Humanized Mice and Convalescent Humans Yields SARS-CoV-2 Antibody Cocktail. Science, doi:in press (2020).
12. Sheng, Z. et al. Gene-Specific Substitution Profiles Describe the Types and Frequencies of Amino Acid Changes during Antibody Somatic Hypermutation. Front Immunol 8, 537, doi:10.3389/fimmu.2017.00537 (2017).
13. ter Meulen, J. et al. Human monoclonal antibody combination against SARS coronavirus: synergy and coverage of escape mutants. Plos Med 3, e237, doi:10.1371/journal.pmed.0030237 (2006).
14. Tian, X. et al. Potent binding of 2019 novel coronavirus spike protein by a SARS coronavirus-specific human monoclonal antibody. Emerg Microbes Infect 9, 382-385, 309doi:10.1080/22221751.2020.1729069 (2020).
15. Yuan, M. et al. A highly conserved cryptic epitope in the receptor binding domains of SARS-CoV-2 and SARS-CoV. Science 368, 630-633, doi:10.1126/science.abb7269 (2020).
16. Rogers, T. F. et al. Rapid isolation of potent SARS-CoV-2 neutralizing antibodies and protection in a small animal model. bioRxiv, doi:10.1101/2020.05.11.088674 (2020).
17. Chen, X. et al. Human monoclonal antibodies block the binding of SARS-CoV-2 spike protein to angiotensin converting enzyme 2 receptor. Cell Mol Immunol 17, 647-649, doi:10.1038/s41423-020-0426-7 (2020).
18. Zeng, X. et al. Isolation of a human monoclonal antibody specific for the receptor binding domain of SARS-CoV-2 using a competitive phage biopanning strategy. Antibody Therapeutics 3, 95-100, doi:10.1093/abt/tbaa008 (2020).
19. Liu, X. et al. Neutralizing Antibodies Isolated by a site-directed Screening have Potent Protection on SARS-CoV-2 Infection. bioRxiv, 2020.2005.2003.074914, doi:10.1101/2020.05.03.074914 (2020).
20. Zost, S. J. et al. Rapid isolation and profiling of a diverse panel of human monoclonal antibodies targeting the SARS-CoV-2 spike protein. bioRxiv, doi:10.1101/2020.05.12.091462 (2020).
21. Robbiani, D. F. et al. Convergent Antibody Responses to SARS-CoV-2 Infection in Convalescent Individuals. bioRxiv, doi:10.1101/2020.05.13.092619 (2020).
22. Brouwer, P. J. M. et al. Potent neutralizing antibodies from COVID-19 patients define multiple targets of vulnerability. bioRxiv, 2020.2005.2012.088716, doi:10.1101/2020.05.12.088716 (2020).
23. Chi, X. et al. A potent neutralizing human antibody reveals the N-terminal domain of the Spike protein of SARS-CoV-2 as a site of vulnerability. bioRxiv, 2020.2005.2008.083964 doi:10.1101/2020.05.08.083964 (2020).
24. Wang, C. et al. A human monoclonal antibody blocking SARS-CoV-2 infection. Nat Commun 11, 2251, doi:10.1038/s41467-020-16256-y (2020).
25. Wang, P. et al. SARS-CoV-2 Neutralizing Antibody Responses Are More Robust in Patients with Severe Disease. bioRxiv, 2020.2006.2013.150250, doi:10.1101/2020.06.13.150250 (2020).
26. Schramm, C. A. et al. SONAR: A High-Throughput Pipeline for Inferring Antibody Ontogenies from Longitudinal Sequencing of B Cell Transcripts. Frontiers in immunology 7, 372, doi:10.3389/fimmu.2016.00372 (2016).
27. Altschul, S. F. et al. Gapped BLAST and PSI-BLAST: a new generation of protein database search programs. Nucleic Acids Res 25, 3389-3402 (1997).
28. Lefranc, M. P. et al. IMGT, the international ImMunoGeneTics information system. Nucleic Acids Res 37, D1006-1012, doi:10.1093/nar/gkn838 (2009).
29. Sievers, F. & Higgins, D. G. Clustal Omega, Accurate Alignment of Very Large Numbers of Sequences. Multiple Sequence Alignment Methods 1079, 105-116, doi:10.1007/978-1-62703-646-7_6 (2014).
30. Edgar, R. C. Search and clustering orders of magnitude faster than BLAST. Bioinformatics 26, 2460-2461, doi:10.1093/bioinformatics/btq461 (2010).
31. Sheng, Z. et al. Gene-Specific Substitution Profiles Describe the Types and Frequencies of Amino Acid Changes during Antibody Somatic Hypermutation. Frontiers in immunology 8, 537 (2017).
32. Nie, J. et al. Establishment and validation of a pseudovirus neutralization assay for SARS-CoV-2. Emerg Microbes Infect 9, 680-686, doi:10.1080/22221751.2020.1743767 (2020).
33. Whitt, M. A. Generation of VSV pseudotypes using recombinant DeltaG-VSV for studies on virus entry, identification of entry inhibitors, and immune responses to vaccines. J Virol Methods 169, 365-374, doi:10.1016/j.jviromet.2010.08.006 (2010).
34. Subway, C. et al. Automated molecular microscopy: the new Leginon system. J Struct Bio1151, 41-60, doi:10.1016/j.jsb.2005.03.010 (2005).
35. Punjani, A., Rubinstein, J. L., Fleet, D. J. & Brubaker, M. A. cryoSPARC: algorithms for rapid unsupervised cryo-EM structure determination. Nat Methods 14, 290-296, doi:10.1038/nmeth.4169 (2017).
36. Bepler, T. et al. Positive-unlabeled convolutional neural networks for particle picking in cryo-electron micrographs. Nature Methods 16, 1153-1160, doi:10.1038/s41592-019-0575-8 (2019).
37. Pettersen, E. F. et al. UCSF Chimera—a visualization system for exploratory research and analysis. J Comput Chem 25, 1605-1612, doi:10.1002/jcc.20084 (2004).
38. Tan, Y. Z. et al. Addressing preferred specimen orientation in single-particle cryo-EM through tilting. Nat Methods 14, 793-796, doi:10.1038/nmeth.4347 (2017).
39. Zheng, S. Q. et al. MotionCor2: anisotropic correction of beam-induced motion for improved cryo-electron microscopy. Nat Methods 14, 331-332, doi:10.1038/nmeth.4193 (2017).
40. Zhu, K. et al. Antibody structure determination using a combination of homology modeling, energy-based refinement, and loop prediction. Proteins 82, 1646-1655, doi:10.1002/prot.24551 (2014).
41. Croll, T. I. ISOLDE: a physically realistic environment for model building into low-resolution 653 electron-density maps. Acta Crystallogr D Struct Biol 74, 519-530, 654 doi: 10.1107/S2059798318002425 (2018).
42. Emsley, P. & Cowtan, K. Coot: model-building tools for molecular graphics. ActaCrystallogr D Biol Crystallogr 60, 2126-2132, doi:10.1107/S0907444904019158 (2004)
43. Adams, P. D. et al. Recent developments in the PHENIX software for automated 658 crystallographic structure determination. J Synchrotron Radiat 11, 53-55, doi:10.1107/s0909049503024130 (2004).
44. Davis, I. W., Murray, L. W., Richardson, J. S. & Richardson, D. C. MOLPROBITY: structure validation and all-atom contact analysis for nucleic acids and their complexes. Nucleic Acids Res 32, W615-619, doi:10.1093/nar/gkh398 (2004).
45. Barad, B. A. et al. EMRinger: side chain-directed model and map validation for 3D cryo-electron microscopy. Nat Methods 12, 943-946, doi:10.1038/nmeth.3541 (2015).
46. Goddard, T. D. et al. UCSF ChimeraX: Meeting modern challenges in visualization and analysis. Protein Sci 27, 14-25, doi:10.1002/pro.3235 (2018).

### Example 11—Potent Neutralizing Monoclonal Antibodies

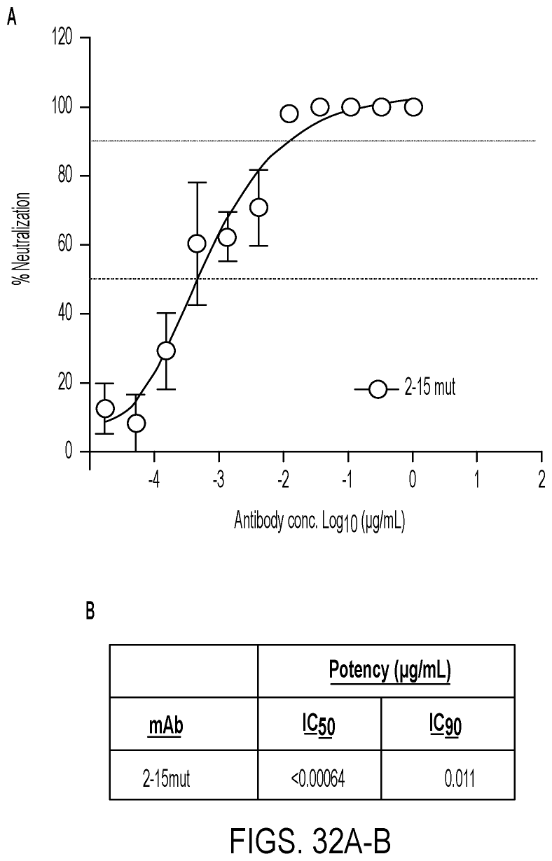

FIGS. 32A-B show monoclonal antibody 2-15 mut. FIG. 32A shows virus neutralization with the 2-15 mut antibody. FIG. 32B shows potency of the 2-15 mut antibody. The 2-15 mut antibody displays an IC₅₀ concentration of less than 0.00064 μg/ml and an IC₉₀ concertation of 0.011 μg/ml.

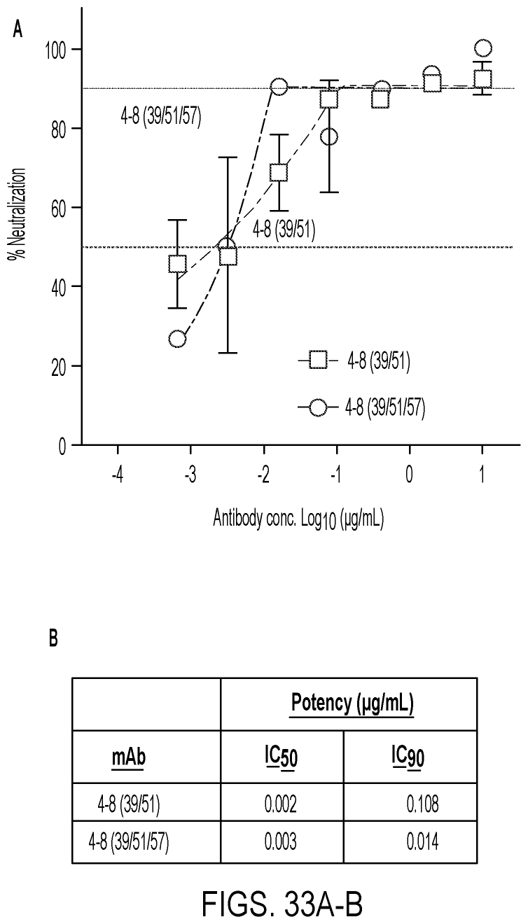

FIGS. 33A-B show select mutants of the 4-8 monoclonal antibody. FIG. 33A shows neutralization with antibodies 4-8 (39/51) and 4-8(39/51/57). FIG. 33B shows potency of antibodies 4-8 (39/51) and 4-8(39/51/57). The 4-8(39/51) antibody displays an IC₅₀ concentration of 0.002 μg/ml and an IC₉₀ concertation of 0.108 μg/ml. The 4-8(39/51/57) antibody displays an IC₅₀ concentration of 0.003 μg/ml and an IC₉₀ concertation of 0.014 μg/ml.

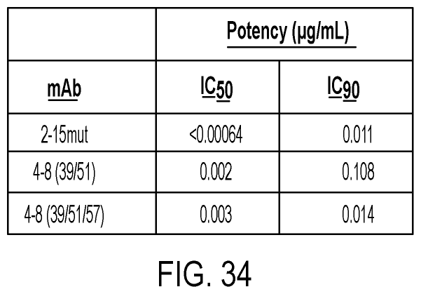

FIG. 34 shows a summary of IC₅₀ and IC₉₀ values of selected monoclonal antibody mutants.

### Example 12—Identification and Characterization of a Cross-Neutralizing Monoclonal Antibody 2-36

SARS-CoV-2 is phylogenetically closely related to SARS-CoV, which caused the 2002-2003 human epidemic. SARS-CoV and SARS-CoV-2 share 76% amino acid identity in their Spike proteins, raising the possibility of conserved immunogenic surfaces on these antigens. Many human monoclonal antibodies have now been shown to target the SARS-CoV-2 Spike protein, but cross-neutralizing antibodies are relatively uncommon in individuals with COVID-19. There are also SARS-like viruses including some zoonotic sarbecoviruses in bats or pangolins which could infect human cells, and therefore, have the potential to cause next pandemics.

Our invention claims to isolate and characterize a monoclonal antibody that could not only neutralize SARS-CoV-2 and SARS-CoV but also neutralize sarbecoviruses in bats and pangolins. Such cross-neutralizing antibodies are extremely valuable to understand how to confer broader protection against human SARS-like viruses that include the extensive reservoir of zoonotic sarbecoviruses in bats, pangolins, etc.

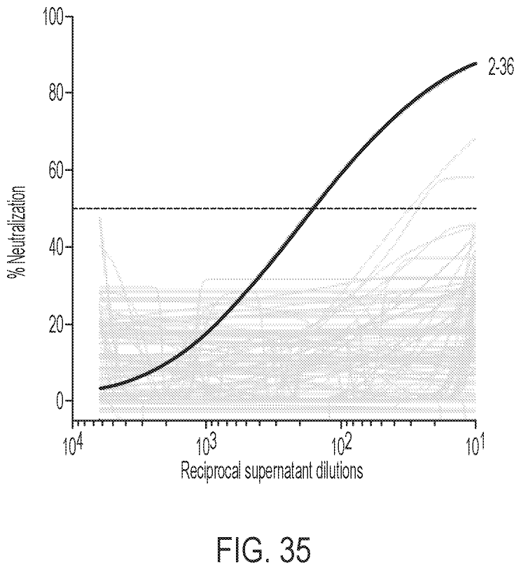

In order to find antibodies that have cross-neutralizing activities, we screened anti-SARS-CoV antibodies from COVID-19 convalescent patients. SARS-CoV-2 spike specific antibodies were isolated by single-B cell sorting and 10X genomics sequencing, and then the antibodies were synthesized and the transfection supernatants were used to screen anti-SARS-CoV antibodies using SARS-CoV pseudovirus. While most of the antibodies showed no or very weak neutralizing activity, antibody 2-36 was found to be a potent neutralizer against SARS-CoV (FIG. 35).

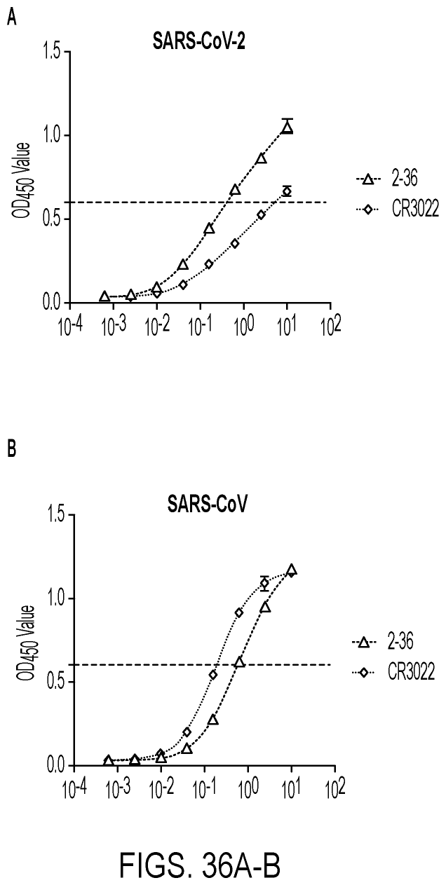

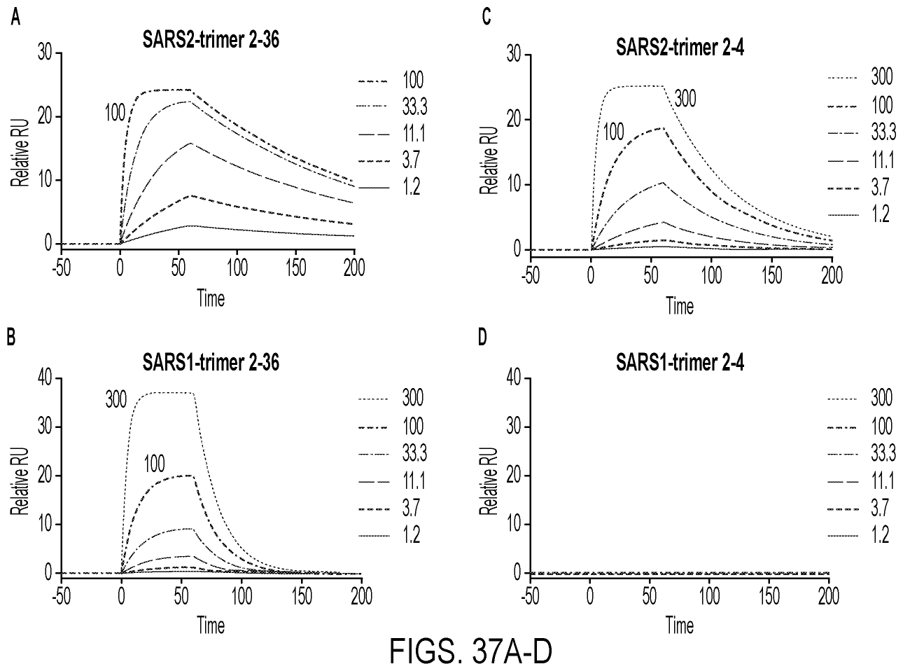

We next characterized this antibody by first testing its binding to SARS-CoV-2 and SARS-CoV spikes by ELISA with CR3022, an antibody previously reported to have cross-activity, as a control. We confirmed that antibody 2-36 can bind to both SARS-CoV-2 and SARS-CoV spikes (FIGS. 36A-B). We then measured the binding affinity of antibody 2-36 to those spikes by SPR and we further confirmed that antibody 2-36 can bind to both SARS-CoV-2 (FIG. 37A) and SARS-CoV (FIG. 37B) spikes but with a higher affinity to SARS-CoV-2 spike. As a control, the SARS-CoV-2 specific antibody 2-4 only binds only to SARS-CoV-2 spike but not to SARS-CoV spike (FIGS. 37C-D).

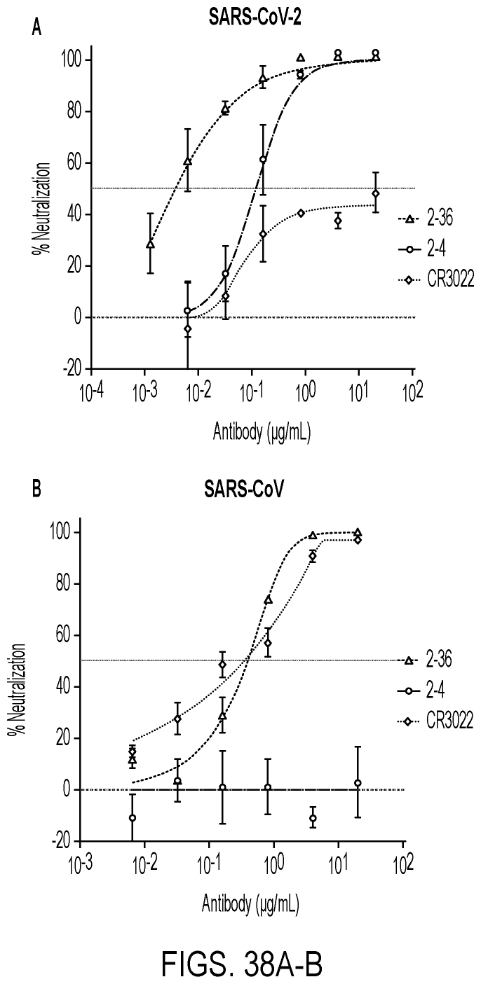

We then tested the neutralization activity of antibody 2-36 against SARS-CoV-2 and SARS-CoV, and showed that antibody 2-36 can potently neutralize SARS-CoV-2 with IC₅₀ 0.004 μg/ml (FIG. 38A). Antibody 2-36 also neutralized SARS-CoV, but with a lower potency, IC₅₀ 0.386 μg/ml (FIG. 38B). The control antibody CR3022 neutralizes SARS-CoV but very weakly on SARS-CoV-2; and antibody 2-4 only neutralizes SARS-CoV-2 (FIGS. 38A-B).

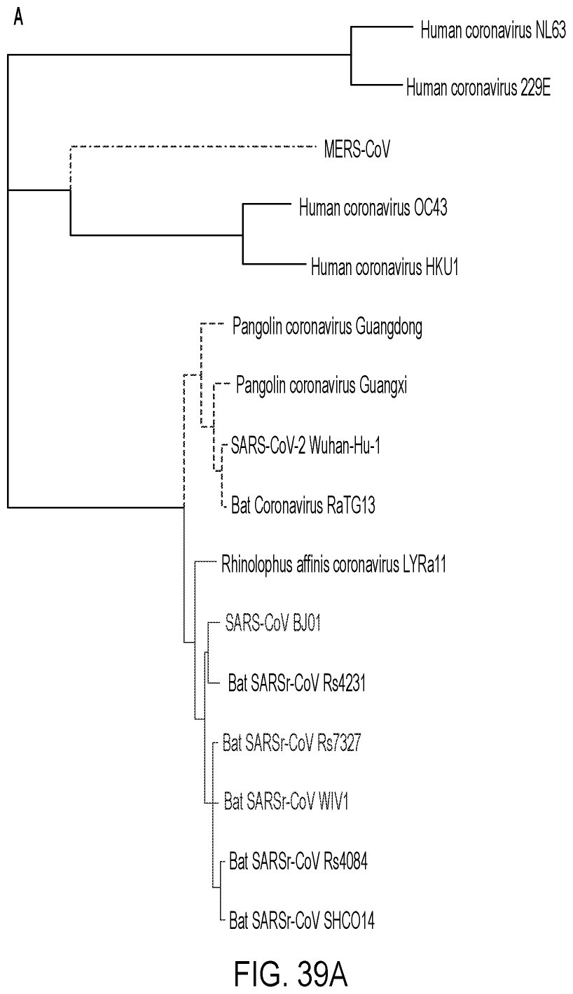

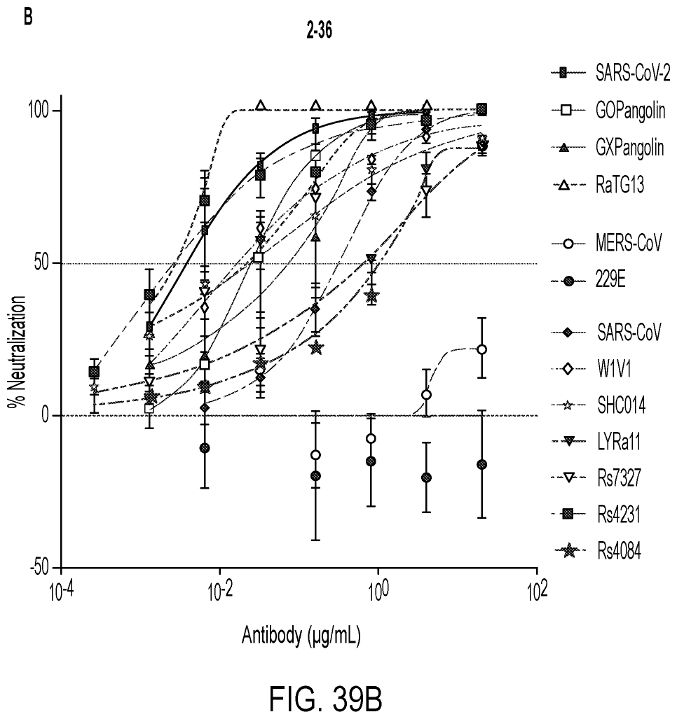

We also tested antibody 2-36 against a panel of other coronaviruses including, MERS-CoV, human common cold coronavirus 229E and some bat and pangolin coronaviruses (FIG. 39A) including 6 among the SARS-CoV-related lineage (e.g., WIV1, SHC014, LYRal 1, Rs7327, Rs4231, Rs4084) and 3 in the SARS-CoV-2-related lineage (e.g., RaTG13, GDPangolin, GXPangolin). Pseudoviruses with the spikes of these coronaviruses were made and tested. As shown in FIG. 39B, antibody 2-36 neutralized SARS-CoV and SARS-CoV-2 and their related lineage viruses, but did not neutralize MERS-CoV and 229E, indicating its breadth.

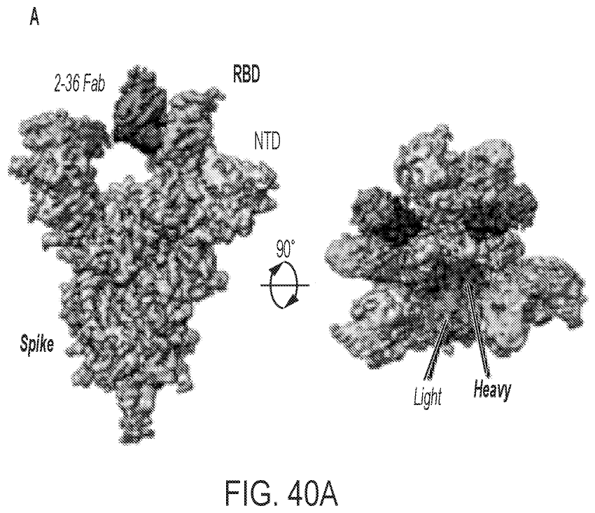

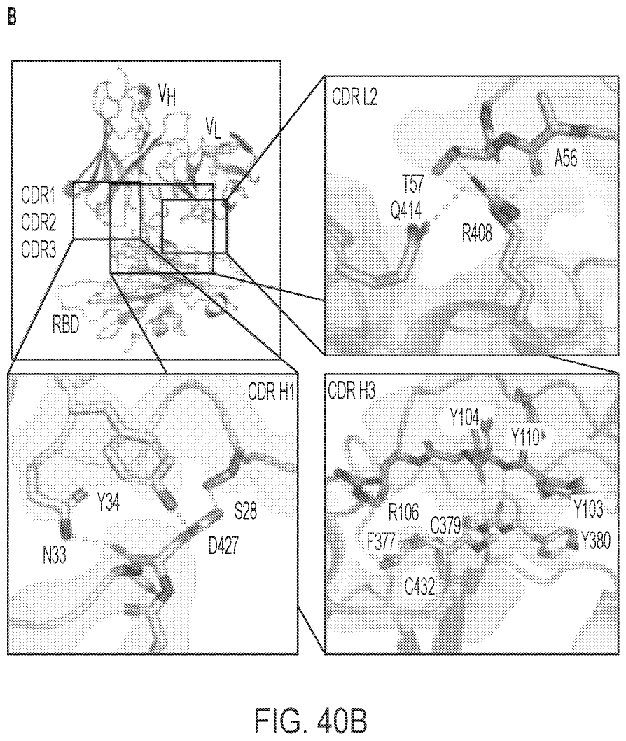

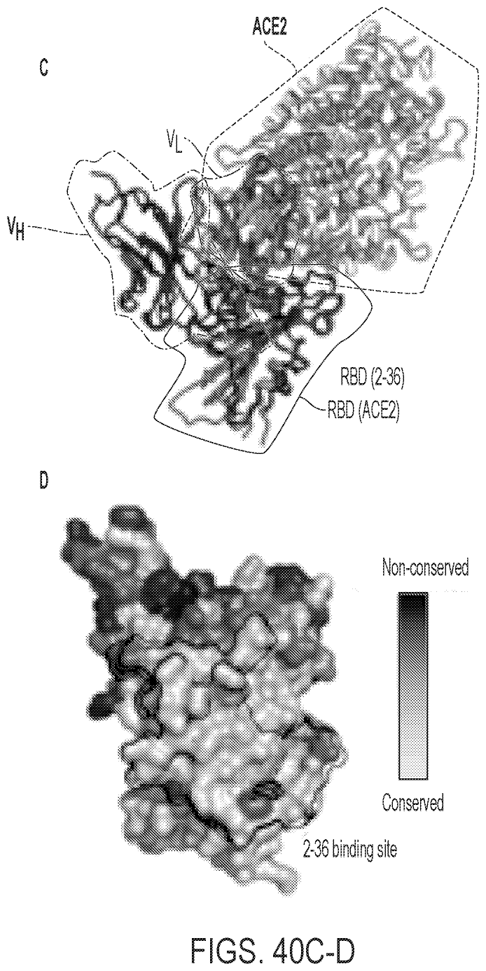

We also solved the Cryo-EM structure of antibody 2-36 in complex with SARS-CoV-2 Spike trimer (FIG. 40A), which showed in detail how the antibody interact with spike (FIG. 40B). 2-36 binds to the side of the RBD and can compete with ACE2 for RBD binding, although its epitope does not overlap the ACE2-binding site (FIG. 40C). The 2-36 epitope on the spike is highly conserved among SARS-CoV-2, SARS-CoV, and other SARS-related coronaviruses (FIG. 40D), which could explain why 2-36 showed such broad neutralizing properties.

As such, antibody 2-36 can serve as the starting point for engineering a more potent antibody against diverse bat coronaviruses with human pandemic potential. Such an antibody could be further developed and placed in the national stockpile for pandemic preparedness. Furthermore, we have a defined a conserved site in the spike that could be targeted for development of a broadly protective vaccine against bat coronaviruses with pandemic potential.

### Example 13 Monoclonal Antibody Number 2-36

Antibody 2-36 is a monoclonal antibody that neutralizes SARS-CoV-2 variants, SARS-CoV, and other beta-coronaviruses. To screen monoclonal antibodies with cross-neutralizing activity against other beta-coronaviruses, more than 200 mAbs isolated from COVID-19 convalescent patients (Liu et al. Nature 2020) were tested on SARS-CoV pseudovirus. Monoclonal antibody 2-36 not only neutralizes SARS-CoV-2 pseudovirus at IC₅₀ ˜0.04 μg/ml and authentic virus at IC50 ˜0.1 μg/mL, but also shows neutralizing activity against SARS-CoV, although with lower potency (IC50 ˜0.2 μg/mL on pseudovirus and IC50 ˜7.5 μg/mL on authentic virus). Monoclonal antibody 2-36 can block SARS-CoV-2 spike trimer binding to ACE2. Conservation analysis on the receptor-binding domain (RBD) among SARS-CoV-2, SARS-CoV, and some bat and pangolin coronavirus strains show that the 2-36 binding site is highly conserved. Antibody 2-36 was tested on different SARS-CoV-2 variants of concern/interest (including B.1.1.7, B.1.351, P.1, B.1.526, R.1, B.1.429) and many single mutations with high circulating frequency. The results show that antibody 2-36 can neutralize all these variants/mutations without any significant loss of activities. Moreover, besides SARS-CoV-2 and SARS-CoV, antibody 2-36 also neutralizes some bat and pangolin coronaviruses which can use human ACE2 as a receptor to enter host cells. These coronaviruses include but are not limited to RaTG13, WIV1, SHC014, GDPangolin, and GXPangolin viruses. Antibody 2-36 escape mutations were selected by in vitro passage of SARS-CoV-2 in Vero E6 cells in presence of 2-36 and confirmed that K378T in spike RBD, with very low frequency in nature, led to viral resistance. Taken these results together, 2-36 represents a promising strategic reserve drug candidate for the prevention and treatment of possible diseases caused by pre-emergent SARS-related CoVs.

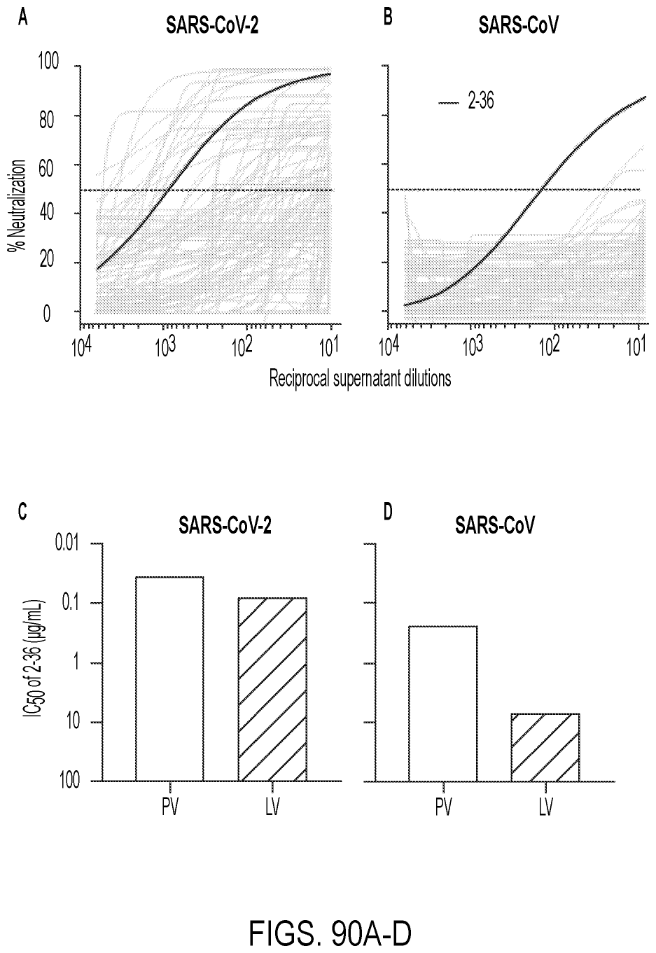

FIGS. 90A-D show virus neutralization with monoclonal antibody 2-36. FIG. 90A shows screening of transfection supernatant for neutralization activity against SARS-CoV-2 pseudovirus. FIG. 90B shows screening of transfection supernatant for neutralization activity against SARS-CoV pseudovirus. FIG. 90C shows 2-36 neutralization IC50 (μg/mL) against SARS-CoV-2 pseudovirus (PV) and live virus (LV). FIG. 90D shows 2-36 neutralization IC50 (μg/mL) against SARS-CoV pseudovirus (PV) and live virus (LV).

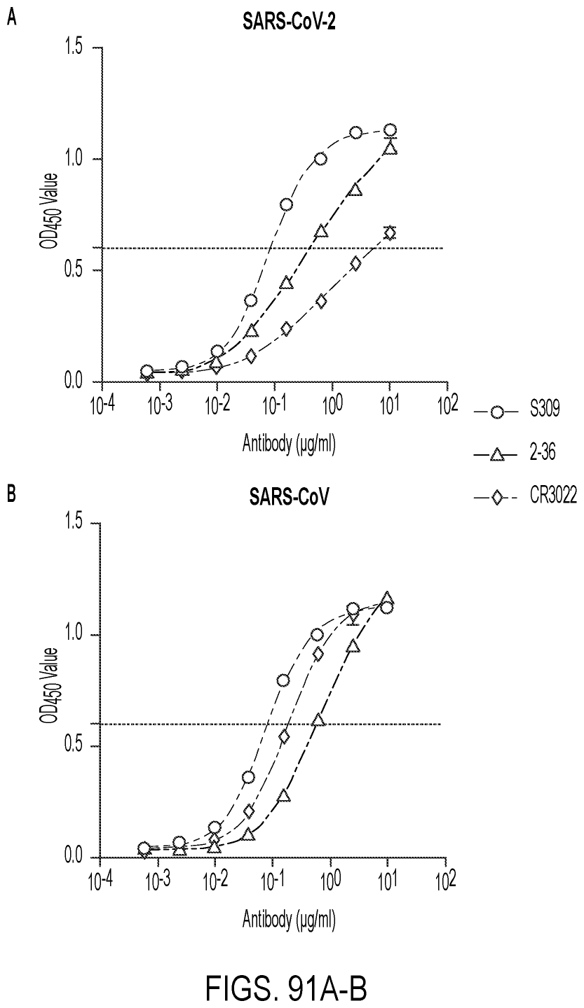

FIGS. 91A-B show binding of monoclonal antibody 2-36 to SARS-CoV-2 (FIG. 91A) and SARS-CoV (FIG. 91B) spike as determined by ELISA.

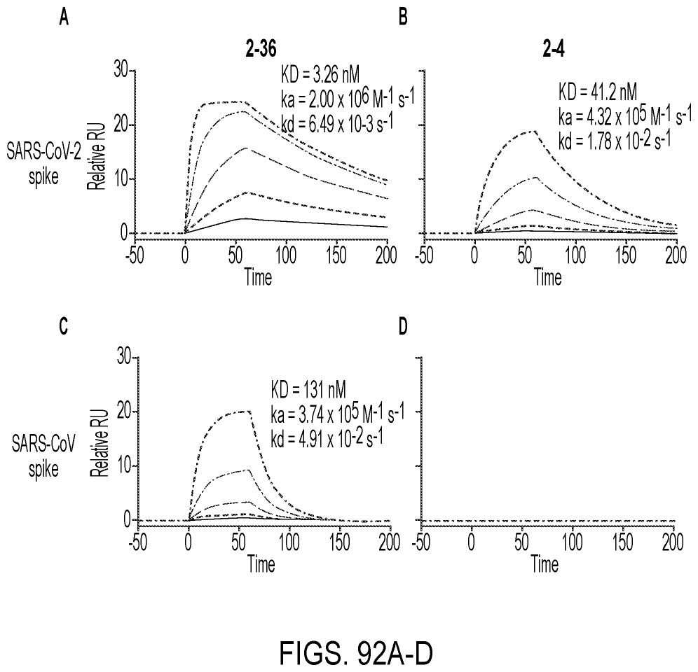

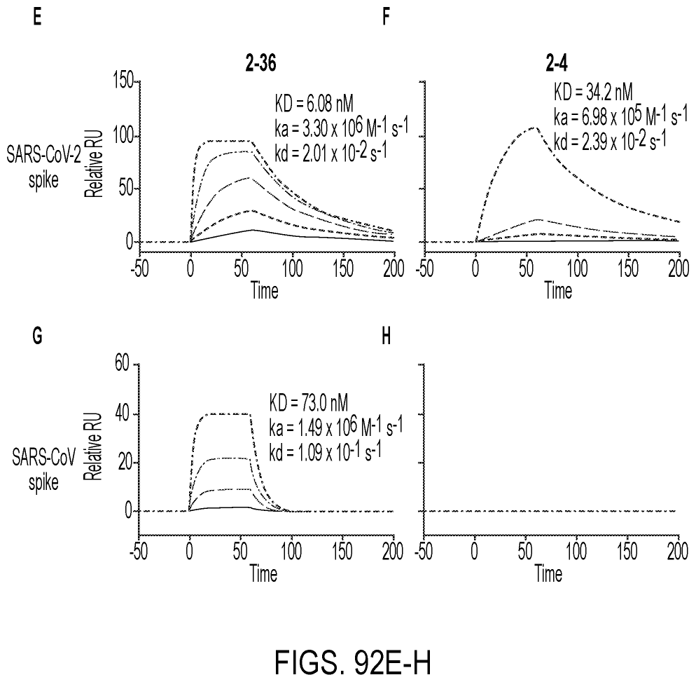

FIGS. 92A-H show binding affinity for monoclonal antibodies 2-36 and 2-4 to SARS-CoV-2 and SARS-CoV viruses as measured by SPR. FIG. 92A shows 2-36 binding affinity to SARS-CoV-2 spike protein. FIG. 92B shows 2-4 binding affinity to SARS-CoV-2 spike protein. FIG. 92C shows 2-36 binding affinity to SARS-CoV spike protein. FIG. 92D shows 2-4 binding affinity to SARS-CoV spike protein. FIG. 92E shows 2-36 binding affinity to SARS-CoV-2 receptor binding domain (RBD). FIG. 92F shows 2-4 binding affinity to SARS-CoV-2 RBD. FIG. 92G shows 2-36 binding affinity to SARS-CoV RBD. FIG. 9211 shows 2-4 binding affinity to SARS-CoV RBD.

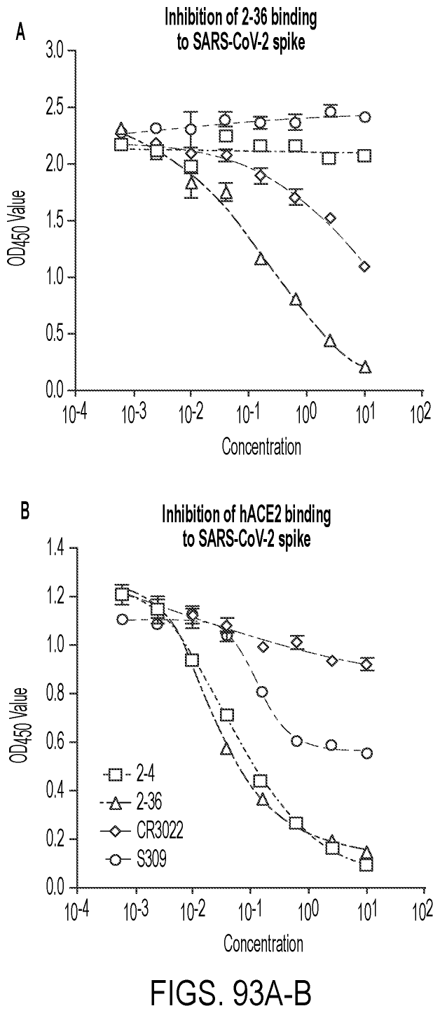

FIGS. 93A-B show binding to SARS-CoV-2 spike protein. FIG. 93A shows that binding of monoclonal antibody 2-36 to SARS-CoV-2 spike is inhibited by CR3022. FIG. 93B shows that monoclonal antibody 2-36 inhibits hACE2 binding to SARS-CoV-2 spike protein.

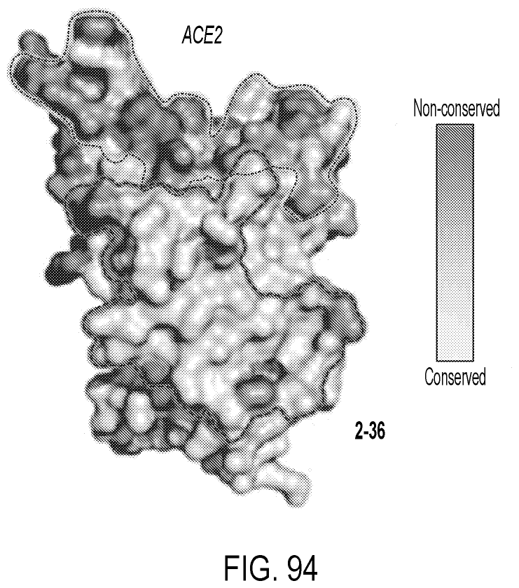

FIG. 94 shows conservation analysis on the RBD among SARS CoV-2, SARS CoV-1, Bat CoVs, and Human CoVs virus strains. The analysis shows that the 2-36 binding site is highly conserved; with regions of high conservation in grey and low conservation in red. The 2-36 binding site is outlined in blue.

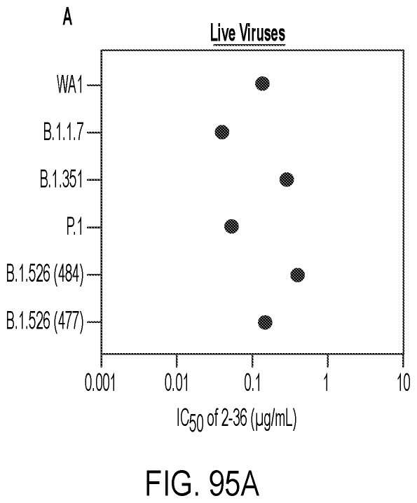

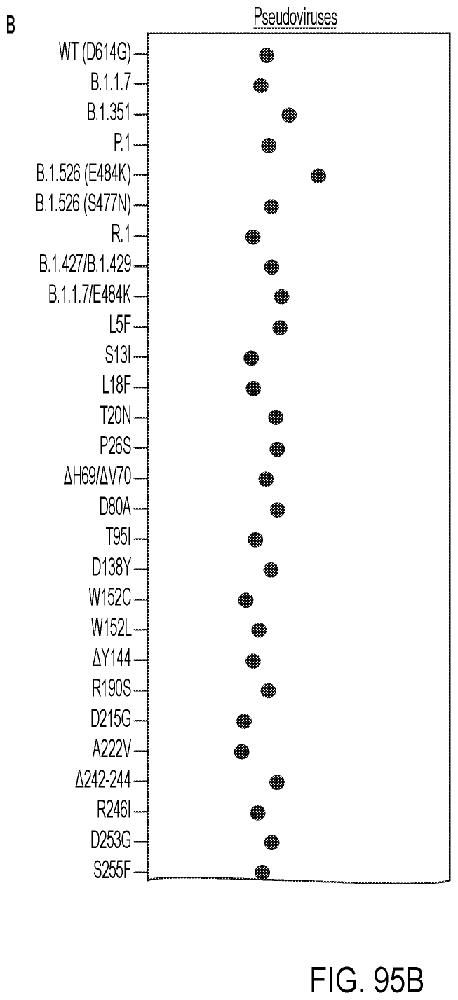

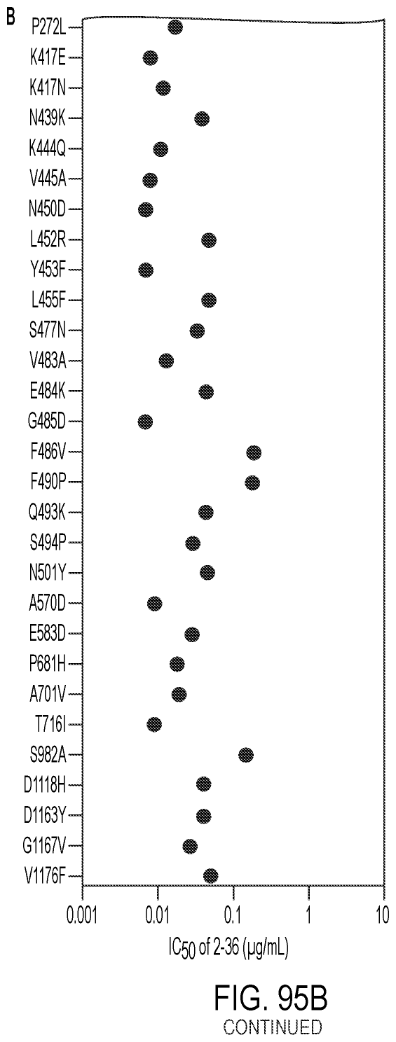

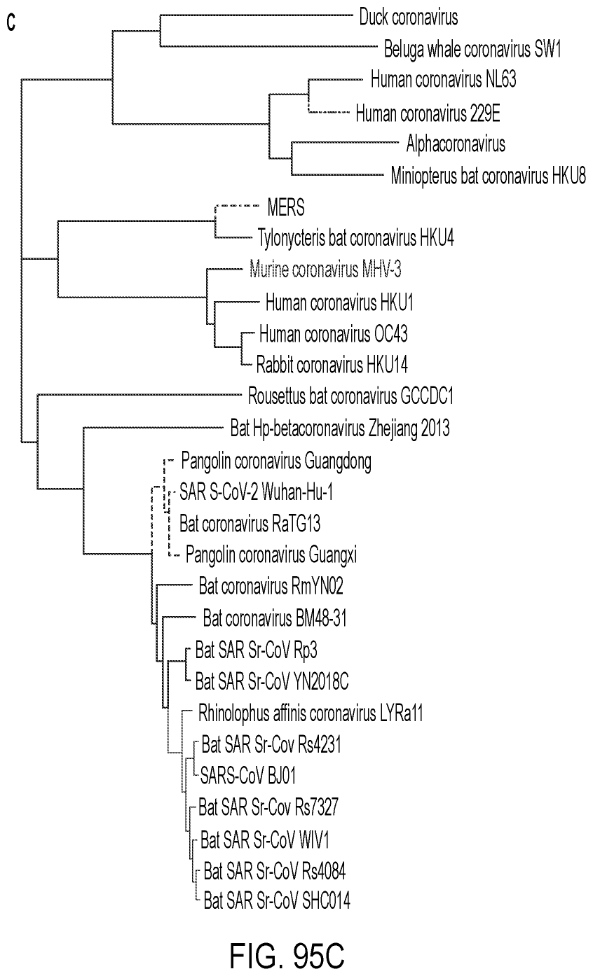

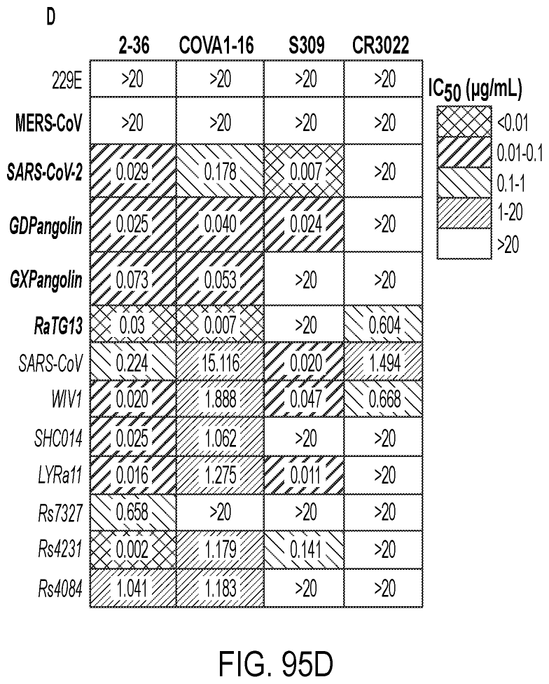

FIGS. 95A-D show that monoclonal antibody 2-36 neutralizes SARS-CoV-2 variants and SARS-like coronaviruses using hACE2. FIG. 95A shows neutralization against live variants. FIG. 95B shows neutralization against pseudovariants. FIG. 95C shows evolution tree of viruses. FIG. 95D shows antibody potency against viruses.

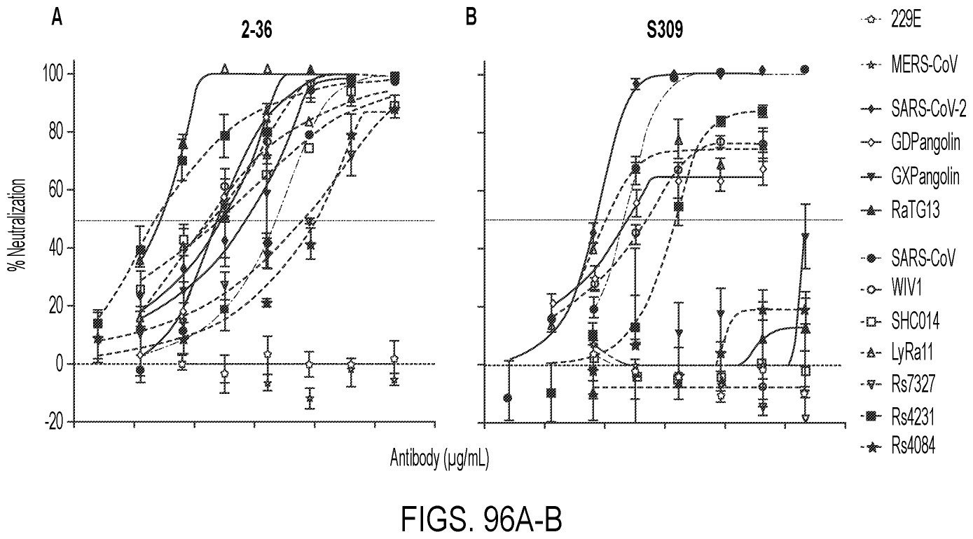

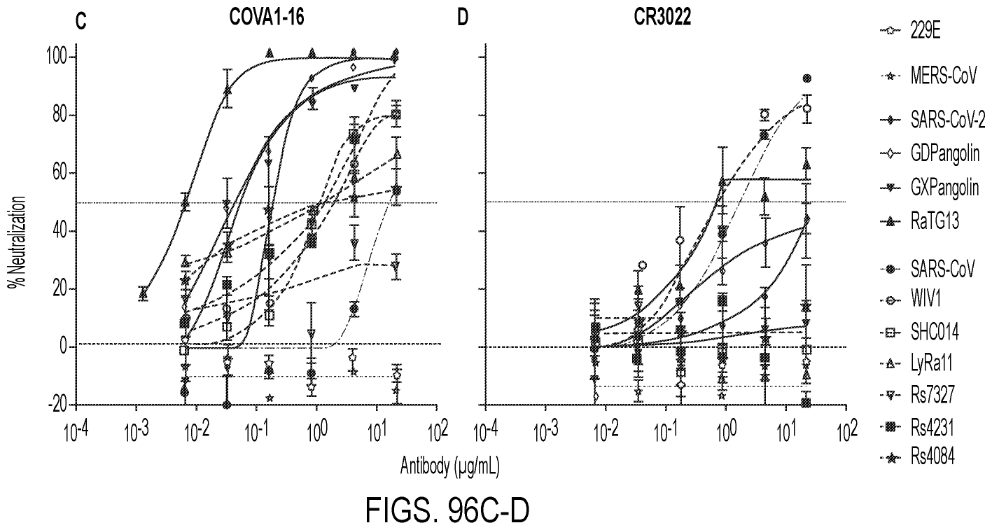

FIGS. 96A-D shows that monoclonal antibody 2-36 neutralizes SARS-like coronaviruses using hACE2. FIG. 96A shows virus neutralization with 2-36 antibody. FIG. 96B shows virus neutralization with S309 antibody. FIG. 96C shows virus neutralization with COVA1-16 antibody. FIG. 96D shows virus neutralization with CR3022 antibody.

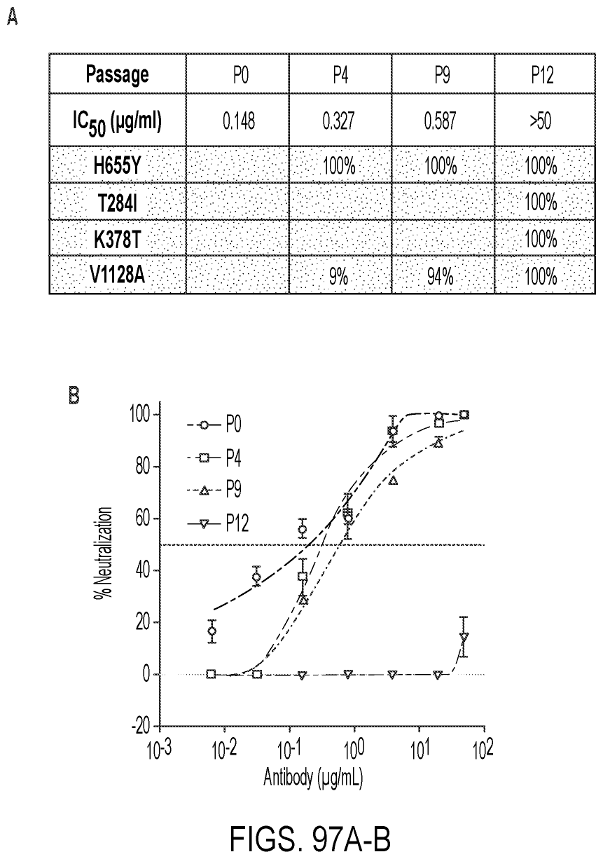

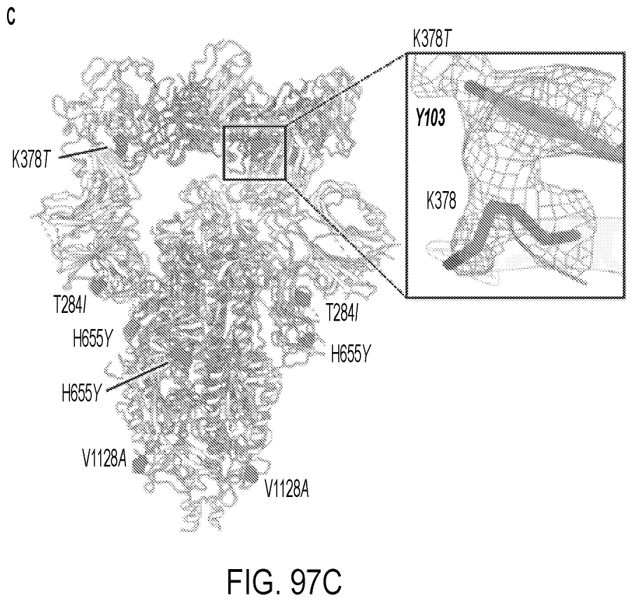

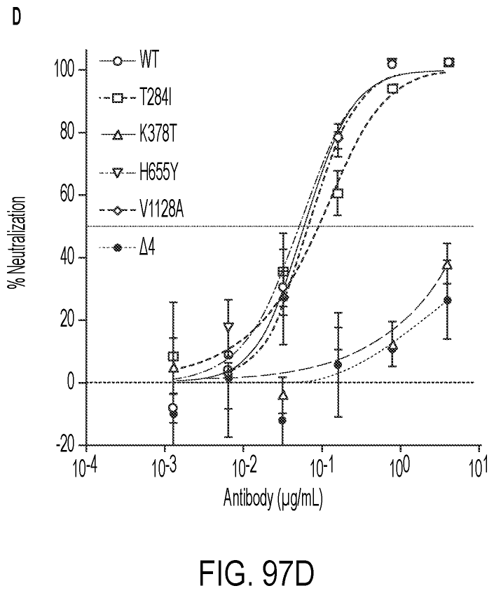

FIGS. 97A-D show in vitro selection of 2-36 antibody escape mutations. FIG. 97A shows evolution of escape mutations during in vitro passage of SARS-CoV-2 USA/WA1 in Vero E6 cells in the presence of 2-36. FIG. 97B shows 2-36 neutralization activity tested against virus passages. FIG. 97C shows spike protein with selected mutation localized. FIG. 97D shows that the selected escape mutations were introduced into pseudoviruses and then 2-36 neutralization activity was tested against these viruses.

### Examples for Bispecific Antibodies

As described herein, bispecific antibodies bearing either the 1400 arm or the A32 arm indicate that those arms of the bispecific antibody are not specific to and do not bind the spike protein of the SARS-CoV-2 virus. These antibodies provide an “irrelevant” control to demonstrate the need for avidity by “both” arms of bispecific antibodies being controlled for. Constructs bearing these irrelevant controls are referred to as single arm IgG controls. 1400 (PGDM1400) is a neutralizing monoclonal antibody against the human immunodeficiency virus (HIV). A32 is a non-neutralizing monoclonal antibody against HIV. The same control antibodies were used throughout the study. AbX and AbY are controls, X=PGDM1400 (HIV neutralization antibody). Y=A32 (HIV neutralization antibody). Because they do not neutralize SARS-CoV-2 or any variant strains, they are utilized herein to construct bispecific antibodies with one arm against SARS-CoV-2 and the other arm without any activity against SARS-CoV-2. These control antibodies, therefore, allow one to accurately attribute the contribution of each arm to the activity of the bispecific antibodies.

### Example 14

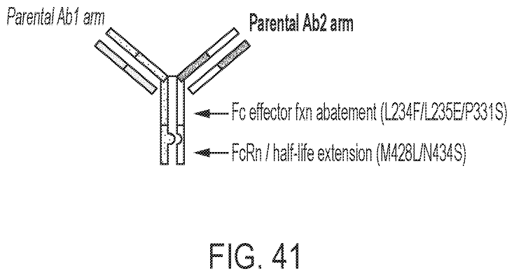

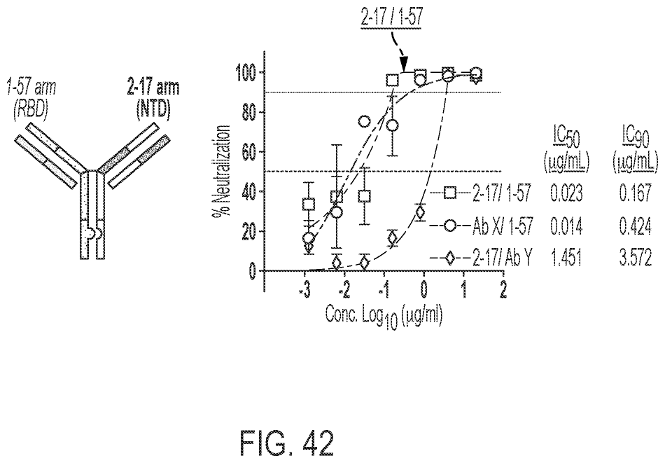

FIG. 42 shows that the 2-17/1-57 bispecific antibody against SARS-CoV-2 does not enhance potency compared to respective single arm counterpart. Ab X and Ab Y are single arm irrelevant controls. X=PGDM1400 (HIV neutralization Ab). Y=A32 (HIV neutralization Ab). The IC₅₀ of the 2-17/1-57 bispecific antibody is 0.023 μg/mL. The IC₉₀ of the 2-17/1-57 bispecific antibody is 0.167 μg/mL. The IC₅₀ of the Ab X/1-57 control is 0.014 μg/mL. The IC₉₀ of the Ab X/1-57 control is 0.424 μg/mL. The IC₅₀ of the 2-17/Ab Y control is 1.451 μg/mL. The IC₉₀ of the 2-17/Ab Y control is 3.572 μg/mL.

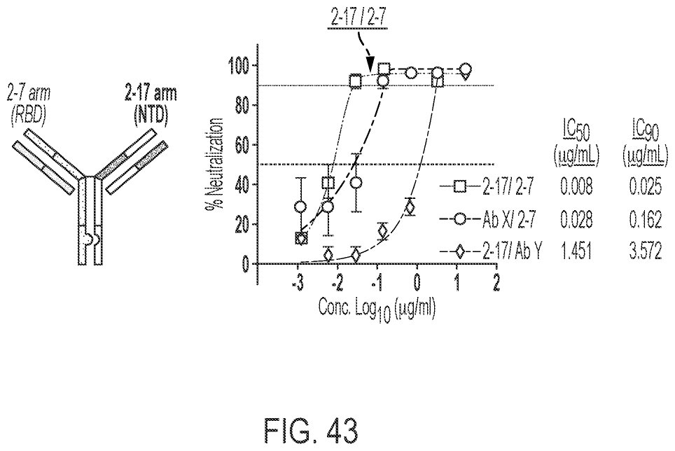

FIG. 43 shows potent neutralization of bispecific antibody 2-17/2-7 against SARS-CoV-2 compared to respective single arm counterpart. Ab X and Ab Y are single arm irrelevant controls. X=PGDM1400 (HIV neutralization Ab). Y=A32 (HIV neutralization Ab). The IC₅₀ of the 2-17/2-7 bispecific antibody is 0.008 μg/mL. The IC₉₀ of the 2-17/2-7 bispecific antibody is 0.025 μg/mL. The IC₅₀ of the Ab X/2-7 control is 0.028 μg/mL. The IC₉₀ of the Ab X/2-7 control is 0.162 μg/mL. The IC₅₀ of the 2-17/Ab Y control is 1.451 μg/mL. The IC₉₀ of the 2-17/Ab Y bispecific antibody is 3.572 μg/mL.

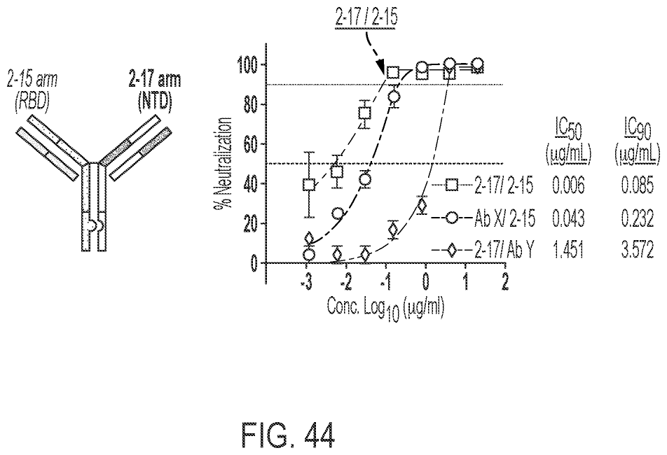

FIG. 44 shows potent neutralization of bispecific antibody 2-17/2-15 against SARS-CoV-2 compared to respective single arm counterpart. Ab X and Ab Y are single arm irrelevant controls. X=PGDM1400 (HIV neutralization Ab). Y=A32 (HIV neutralization Ab). The IC₅₀ of the 2-17/2-15 bispecific antibody is 0.006 μg/mL. The IC₉₀ of the 2-17/2-15 bispecific antibody is 0.085 μg/mL. The IC₅₀ of the Ab X/2-15 control is 0.043 μg/mL. The IC₉₀ of the Ab X/2-15 bispecific antibody is 0.232 μg/mL. The IC₅₀ of the 2-17/Ab Y control is 1.451 μg/mL. The IC₉₀ of the 2-17/Ab Y control is 3.572 μg/mL.

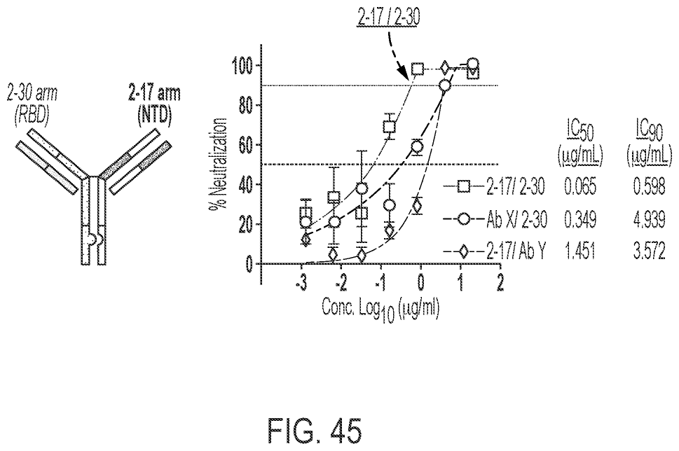

FIG. 45 shows potent neutralization of bispecific antibody 2-17/2-30 against SARS-CoV-2 compared to respective single arm counterpart. Ab X and Ab Y are single arm irrelevant controls. X=PGDM1400 (HIV neutralization Ab). Y=A32 (HIV neutralization Ab). The IC₅₀ of the 2-17/2-30 bispecific antibody is 0.065 μg/mL. The IC₉₀ of the 2-17/2-30 bispecific antibody is 0.598 μg/mL. The IC₅₀ of the Ab X/2-30 control is 0.349 μg/mL. The IC₉₀ of the Ab X/2-30 control is 4.939 μg/mL. The IC₅₀ of the 2-17/Ab Y control is 1.451 μg/mL. The IC₉₀ of the 2-17/Ab Y control is 3.572 μg/mL.

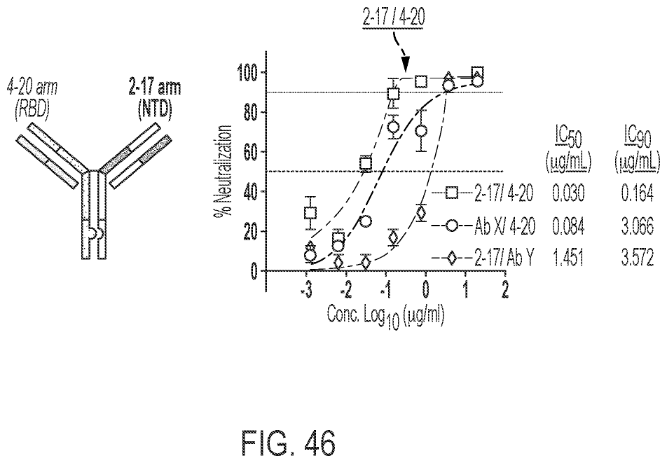

FIG. 46 shows potent neutralization of bispecific antibody 2-17/4-20 against SARS-CoV-2 compared to respective single arm counterpart. Ab X and Ab Y are single arm irrelevant controls. X=PGDM1400 (HIV neutralization Ab). Y=A32 (HIV neutralization Ab). The IC₅₀ of the 2-17/4-20 bispecific antibody is 0.030 μg/mL. The IC₉₀ of the 2-17/4-20 bispecific antibody is 0.164 μg/mL. The IC₅₀ of the Ab X/4-20 control is 0.084 μg/mL. The IC₉₀ of the Ab X/4-20 control is 3.066 μg/mL. The IC₅₀ of the 2-17/Ab Y control is 1.451 μg/mL. The IC₉₀ of the 2-17/Ab Y control is 3.572 μg/mL.

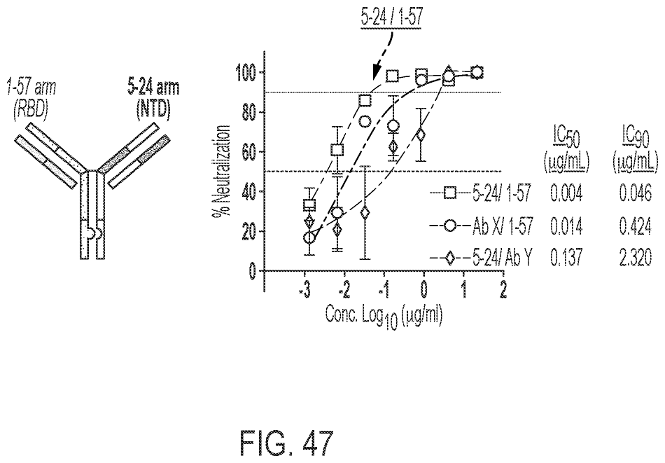

FIG. 47 shows potent neutralization of bispecific antibody 5-24/1-57 against SARS-CoV-2 compared to respective single arm counterpart. Ab X and Ab Y are single arm irrelevant controls. X=PGDM1400 (HIV neutralization Ab). Y=A32 (HIV neutralization Ab). The IC₅₀ of the 5-24/1-57 bispecific antibody is 0.004 μg/mL. The IC₉₀ of the 5-24/1-57 bispecific antibody is 0.046 μg/mL. The IC₅₀ of the Ab X/1-57 control is 0.014 μg/mL. The IC₉₀ of the Ab X/1-57 control is 0.424 μg/mL. The IC₅₀ of the 5-24/Ab Y control is 0.137 μg/mL. The IC₉₀ of the 5-24/Ab Y control is 2.320 μg/mL.

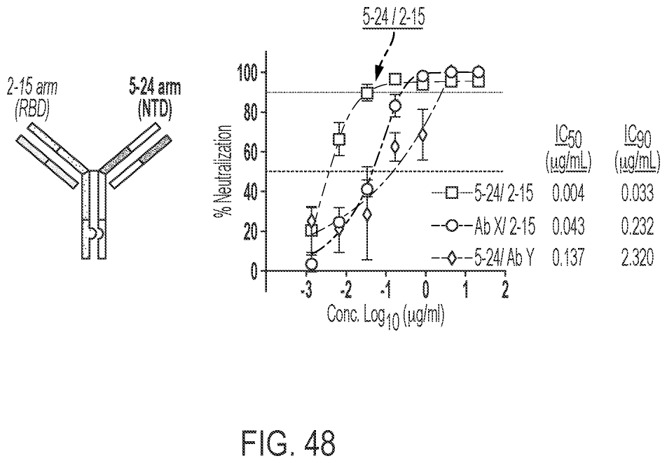

FIG. 48 shows potent neutralization of bispecific antibody 5-24/2-15 against SARS-CoV-2 compared to respective single arm counterpart. Ab X and Ab Y are single arm irrelevant controls. X=PGDM1400 (HIV neutralization Ab). Y=A32 (HIV neutralization Ab). The IC₅₀ of the 5-24/2-15 bispecific antibody is 0.004 μg/mL. The IC₉₀ of the 5-24/2-15 bispecific antibody is 0.033 μg/mL. The IC₅₀ of the Ab X/2-15 control is 0.043 μg/mL. The IC₉₀ of the ab X/2-15 control is 0.232 μg/mL. The IC₅₀ of the 5-24/Ab Y control is 0.137 μg/mL. The IC₉₀ of the 5-24/Ab Y control is 2.320 μg/mL.

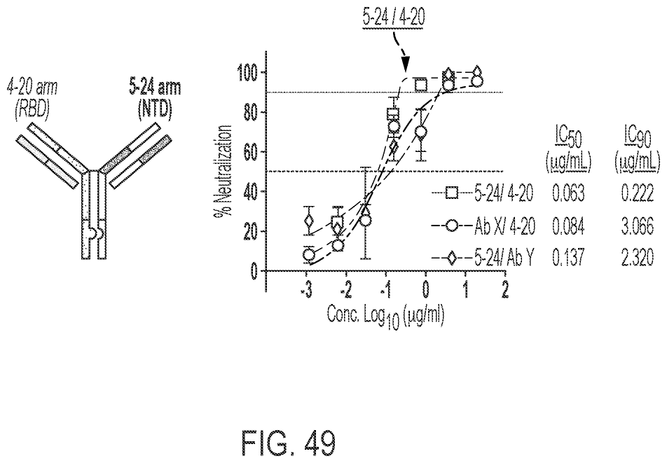

FIG. 49 shows bispecific antibody 5-24/4-20 against SARS-CoV-2 does not enhance potency (IC₅₀) compared to respective single arm counterpart. Ab X and Ab Y are single arm irrelevant controls. X=PGDM1400 (HIV neutralization Ab). Y=A32 (HIV neutralization Ab). The IC₅₀ of the 5-24/4-20 bispecific antibody is 0.063 μg/mL. The IC₉₀ of the 5-24/4-20 bispecific antibody is 0.222 μg/mL. The IC₅₀ of the Ab X/4-20 control is 0.084 μg/mL. The IC₉₀ of the Ab X/4-20 control is 3.066 μg/mL. The IC₅₀ of the 5-24/Ab Y control is 0.137 μg/mL. The IC₉₀ of the 5-24/Ab Y control is 2.320 μg/mL.

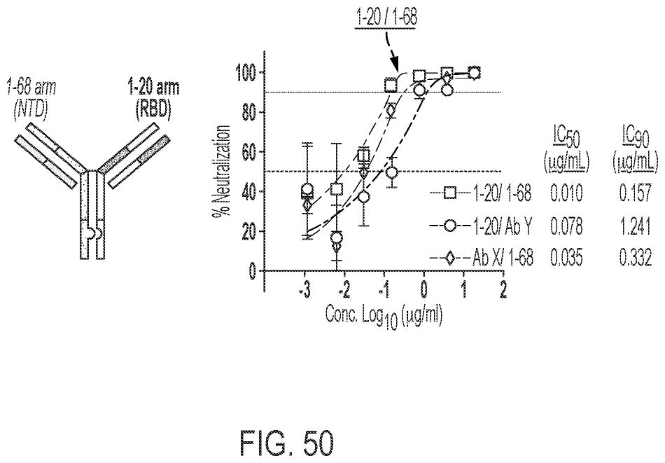

FIG. 50 shows potent neutralization of bispecific antibody 1-20/1-68 against SARS-CoV-2 compared to respective single arm counterpart. Ab X and Ab Y are single arm irrelevant controls. X=PGDM1400 (HIV neutralization Ab). Y=A32 (HIV neutralization Ab). The IC₅₀ of the 1-20/1-68 bispecific antibody is 0.010 μg/mL. The IC₉₀ of the 1-20/1-68 bispecific antibody is 0.157 μg/mL. The IC₅₀ of the 1-20/Ab Y control is 0.078 μg/mL. The IC₉₀ of the 1-20/Ab Y control is 1.241 μg/mL. The IC₅₀ of the Ab X/1-68 control is 0.035 μg/mL. The IC₉₀ of the Ab X/1-68 control is 0.332 μg/mL.

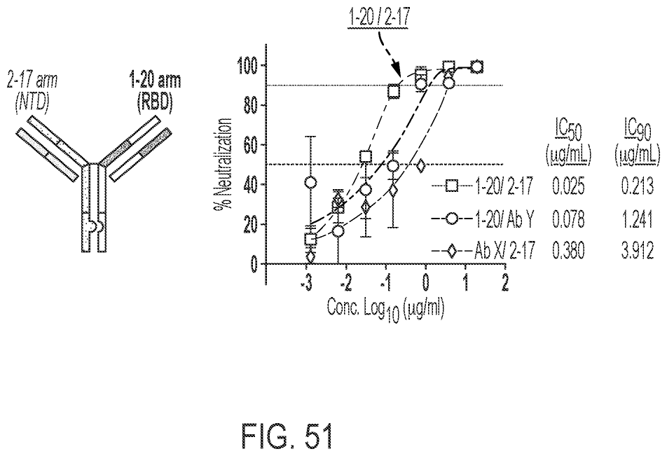

FIG. 51 shows potent neutralization of bispecific antibody 1-20/2-17 against SARS-CoV-2 compared to respective single arm counterpart. Ab X and Ab Y are single arm irrelevant controls. X=PGDM1400 (HIV neutralization Ab). Y=A32 (HIV neutralization Ab). The IC₅₀ of the 1-20/2-17 bispecific antibody is 0.025 μg/mL. The IC₉₀ of the 1-20/2-17 bispecific antibody is 0.213 μg/mL. The IC₅₀ of the 1-20/Ab Y control is 0.078 μg/mL. The IC₉₀ of the 1-20/Ab Y control is 1.241 μg/mL. The IC₅₀ of the Ab X/2-17 control is 0.380 μg/mL. The IC₉₀ of the Ab X/2-17 control is 3.912 μg/mL.

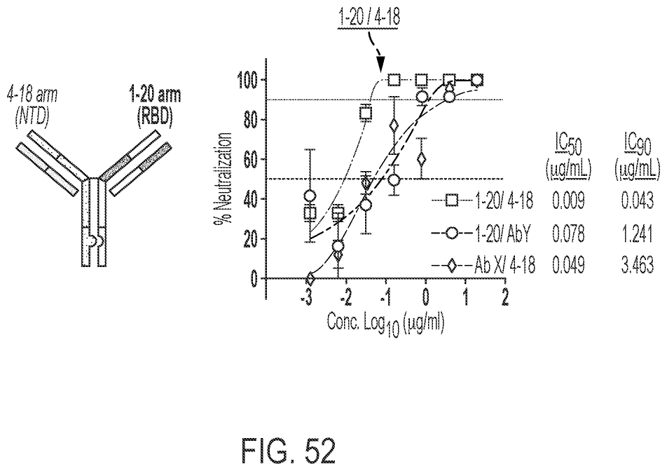

FIG. 52 shows potent neutralization of bispecific antibody 1-20/4-18 against SARS-CoV-2 compared to respective single arm counterpart. Ab X and Ab Y are single arm irrelevant controls. X=PGDM1400 (HIV neutralization Ab). Y=A32 (HIV neutralization Ab). The IC₅₀ of the 1-20/4-18 bispecific antibody is 0.009 μg/mL. The IC₉₀ of the 1-20/4-18 bispecific antibody is 0.043 μg/mL. The IC₅₀ of the 1-20/Ab Y control is 0.078 μg/mL. The IC₉₀ of the 1-20/Ab Y control is 1.241 μg/mL. The IC₅₀ of the Ab X/4-18 control is 0.049 μg/mL. The IC₉₀ of the Ab X/4-18 control is 3.463 μg/mL.

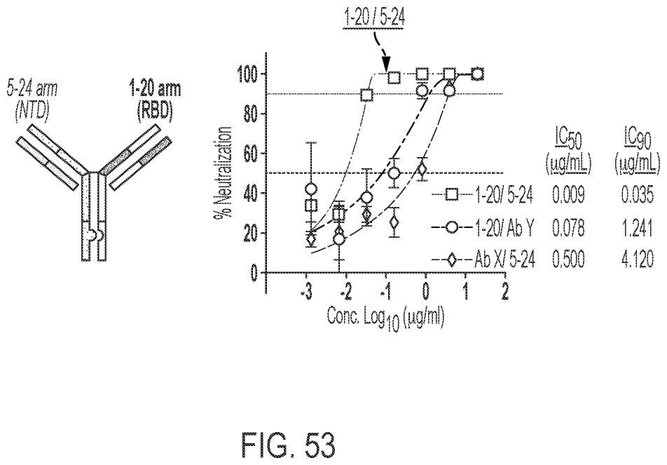

FIG. 53 shows potent neutralization of bispecific antibody 1-20/5-24 against SARS-CoV-2 compared to respective single arm counterpart. Ab X and Ab Y are single arm irrelevant controls. X=PGDM1400 (HIV neutralization Ab). Y=A32 (HIV neutralization Ab). The IC₅₀ of the 1-20/5-24 bispecific antibody is 0.009 μg/mL. The IC₉₀ of the 1-20/5-24 bispecific antibody is 0.035 μg/mL. The IC₅₀ of the 1-20/Ab Y control is 0.078 μg/mL. The IC₉₀ of the 1-20/Ab Y control is 1.241 μg/mL. The IC₅₀ of the Ab X/5-24 control is 0.500 μg/mL. The IC₉₀ of the Ab X/5-24 control is 4.120 μg/mL.

Constructed bispecific antibodies show potent neutralization against SARS-CoV-2 at a level similar or better than the parental antibodies. Both arms of the bispecific antibodies contribute to the neutralization activity of the bispecific molecule. A bispecific antibody is a single molecule with high barrier for viral escape and/or synergetic potency for COVID-19 treatment and prevention. Activity enhancement observed can be extended to any engineered bispecific antibody format.

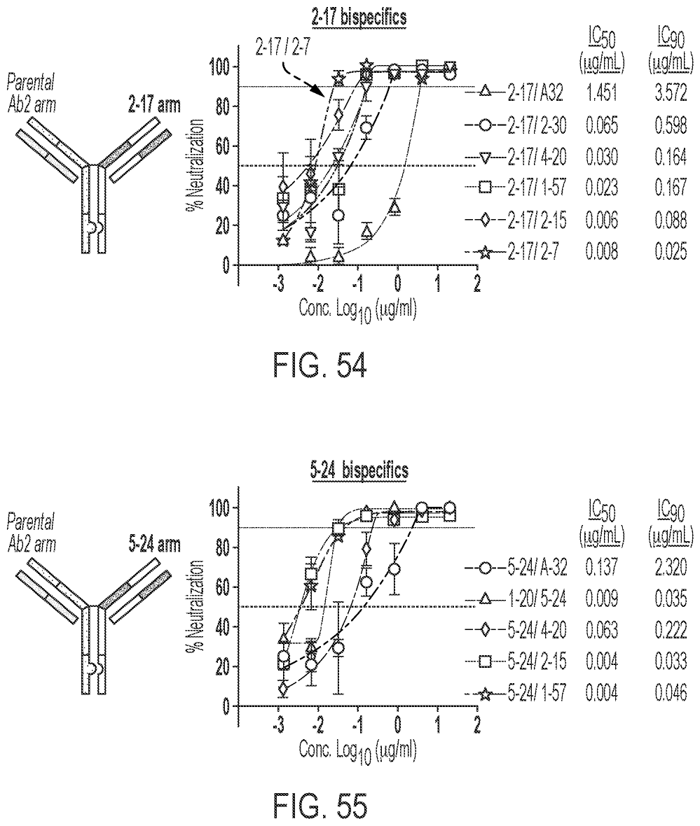

FIG. 54 shows potent neutralization against SARS-CoV-2. Bispecific antibodies with 2-17 as a component are more potent than the single arm control (2-17/A32, A32 is a control antibody show no activity against SARS-CoV-2), indicating that the other arm targeting the different viral epitope together with the 2-17 arm are contributing to the overall neutralization activity of the bispecific molecule. FIG. 54 shows IC₅₀ and IC₉₀ values for 2-17-containing bispecific antibodies.

FIG. 55 shows potent neutralization against SARS-CoV-2 with bispecific antibodies containing 5-24 as a component. FIG. 55 shows IC₅₀ and IC₉₀ values for 5-24-containing bispecific antibodies.

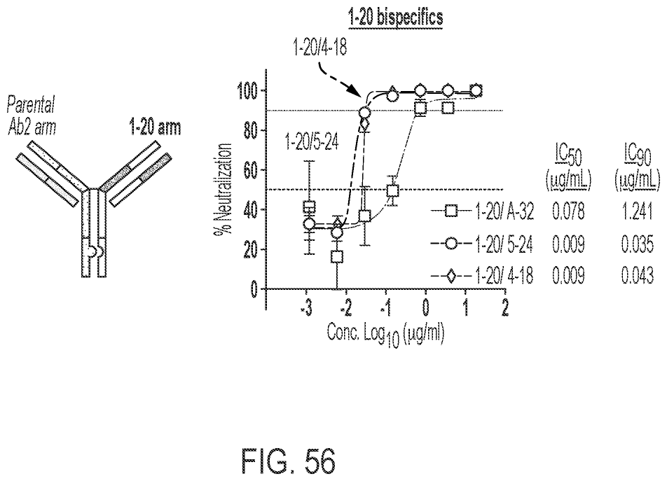

FIG. 56 shows potent neutralization against SARS-CoV-2 with bispecific antibodies containing 1-20 as a component. FIG. 56 shows IC₅₀ and IC₉₀ values for 1-20-containing bispecific antibodies.

### Example 15

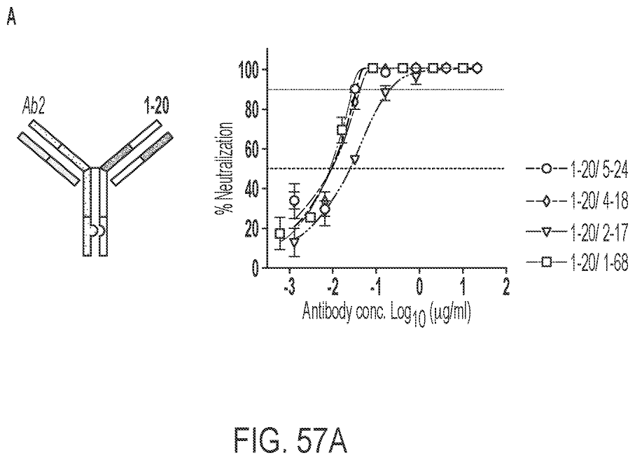

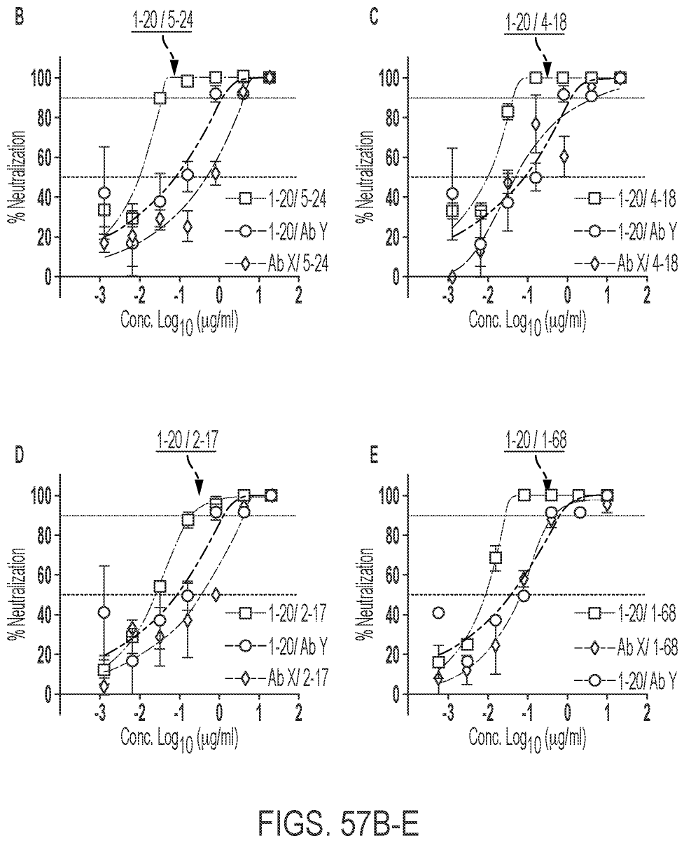

FIGS. 57A-E show bispecific antibodies with 1-20 (RBD) as a parental antibody. FIG. 57A shows virus neutralization with bispecific antibodies 1-20/5-24, 1-20/4-18, 1-20/2-17, and 1-20/1-68. FIG. 57B shows virus neutralization with the bispecific antibody 1-20/5-24 compared to control antibodies. FIG. 57C shows virus neutralization with the bispecific antibody 1-20/4-18 compared to control antibodies. FIG. 57D shows virus neutralization with the bispecific antibody 1-20/2-17 compared to control antibodies. FIG. 57E shows virus neutralization with the bispecific antibody 1-20/1-68 compared to control antibodies. Control antibodies were generated using irrelevant antibody such as PGDM1400 (Ab X) or A32 (Ab Y) replacing one arm of the bispecific antibody.

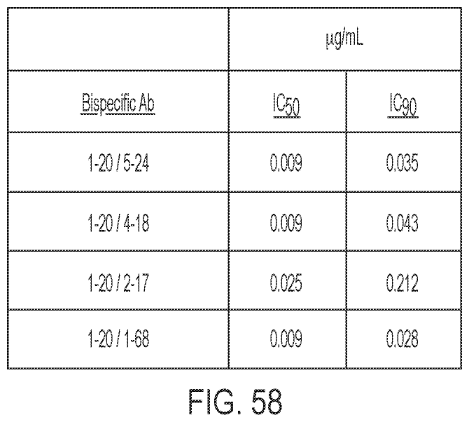

FIG. 58 shows potencies of bispecific antibodies with 1-20 (RBD) as a parental antibody. The IC₅₀ of bispecific antibody 1-20/5-24 is 0.009 μg/ml. The IC₉₀ of bispecific antibody 1-20/5-24 is 0.035 μg/ml. The IC₅₀ of bispecific antibody 1-20/4-18 is 0.009 μg/ml. The IC₉₀ of bispecific antibody 1-20/4-18 is 0.043 μg/ml. The IC₅₀ of bispecific antibody 1-20/2-17 is 0.025 μg/ml. The IC₉₀ of bispecific antibody 1-20/2-17 is 0.212 μg/ml. The IC₅₀ of bispecific antibody 1-20/1-68 is 0.009 μg/ml. The IC₉₀ of bispecific antibody 1-20/1-68 is 0.028 μg/ml.

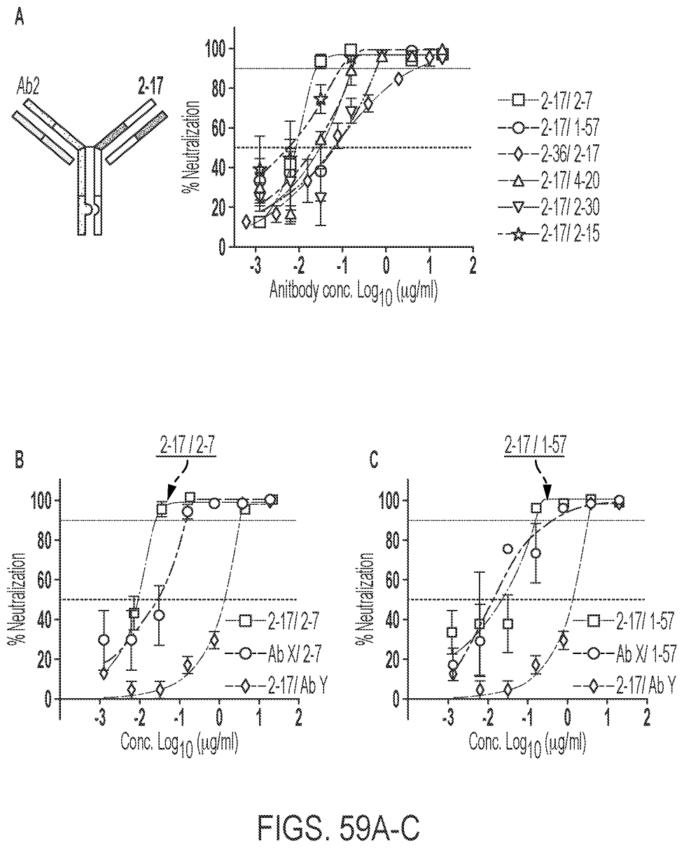

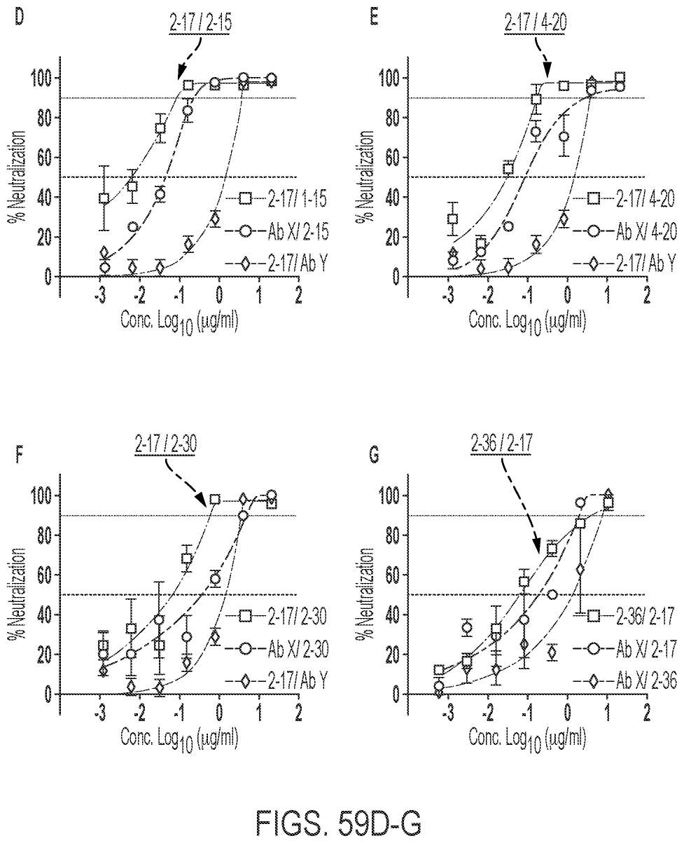

FIGS. 59A-G show bispecific antibodies with 2-17 (NTD) as a parental antibody. FIG. 59A shows virus neutralization with bispecific antibodies 2-17/2-7, 2-17/1-57, 2-36/2-17, 2-17/4-20, 2-17/2-30, and 2-17/2-15. FIG. 59B shows virus neutralization with the bispecific antibody 2-17/2-7 compared to control antibodies. FIG. 59C shows virus neutralization with the bispecific antibody 2-17/1-57 compared to control antibodies. FIG. 59D shows virus neutralization with the bispecific antibody 2-17/2-15 compared to control antibodies. FIG. 59E shows virus neutralization with the bispecific antibody 2-17/4-20 compared to control antibodies. FIG. 59F shows virus neutralization with the bispecific antibody 2-17/2-30 compared to control antibodies. FIG. 59G shows virus neutralization with the bispecific antibody 2-36/2-17 compared to control antibodies.

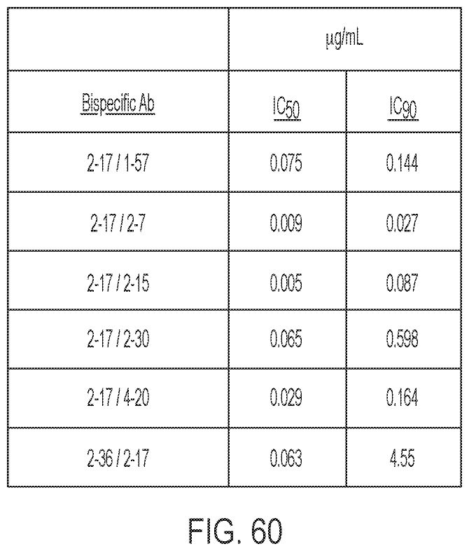

FIG. 60 shows potencies of bispecific antibodies with 2-17 (NTD) as a parental antibody. The IC₅₀ of bispecific antibody 2-17/1-57 is 0.075 μg/ml. The IC₉₀ of bispecific antibody 2-17/1-57 is 0.144 μg/ml. The IC₅₀ of bispecific antibody 2-17/2-7 is 0.009 μg/ml. The IC₉₀ of bispecific antibody 2-17/2-7 is 0.027 μg/ml. The IC₅₀ of bispecific antibody 2-17/2-15 is 0.005 μg/ml. The IC₉₀ of bispecific antibody 2-17/2-15 is 0.087 μg/ml. The IC₅₀ of bispecific antibody 2-17/2-30 is 0.065 μg/ml. The IC₉₀ of bispecific antibody 2-17/2-30 is 0.598 μg/ml. The IC₅₀ of bispecific antibody 2-17/4-20 is 0.029 μg/ml. The IC₉₀ of bispecific antibody 2-17/4-20 is 0.164 μg/ml. The IC₅₀ of bispecific antibody 2-36/2-17 is 0.063 μg/ml. The IC₉₀ of bispecific antibody 2-36/2-17 is 4.55 μg/ml.

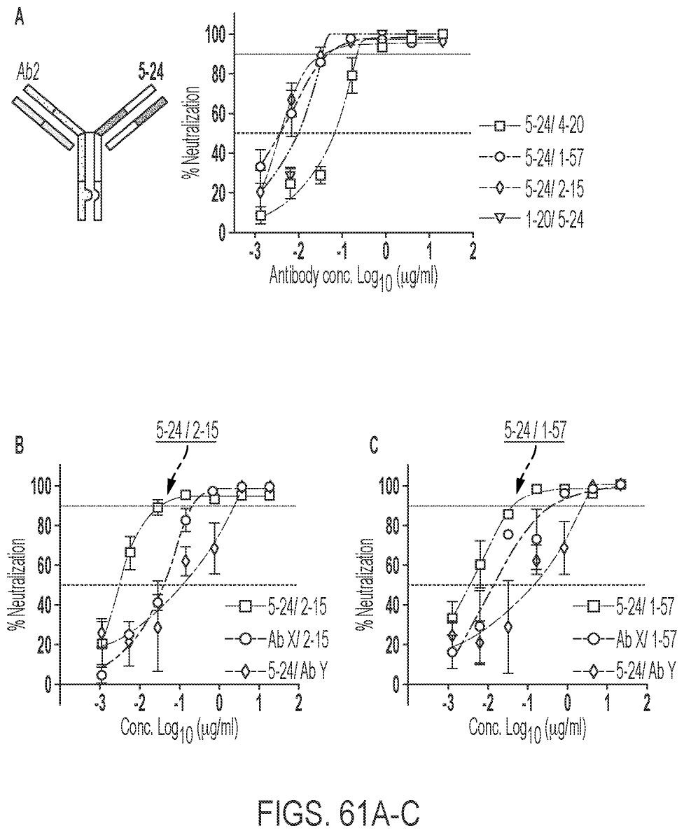

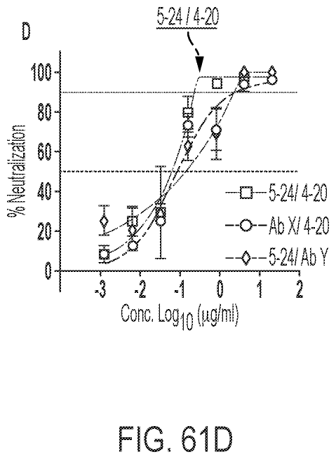

FIGS. 61A-D show bispecific antibodies with 5-24 (NTD) as a parental antibody. FIG. 61A shows virus neutralization with bispecific antibodies 5-24/4-20, 5-24/1-57, 5-24/2-15, and 1-20/5-24. FIG. 61B shows virus neutralization with the bispecific antibody 5-24/2-15 compared to control antibodies. FIG. 61C shows virus neutralization with the bispecific antibody 5-24/1-57 compared to control antibodies. FIG. 61D shows virus neutralization with the bispecific antibody 5-24/4-20 compared to control antibodies.

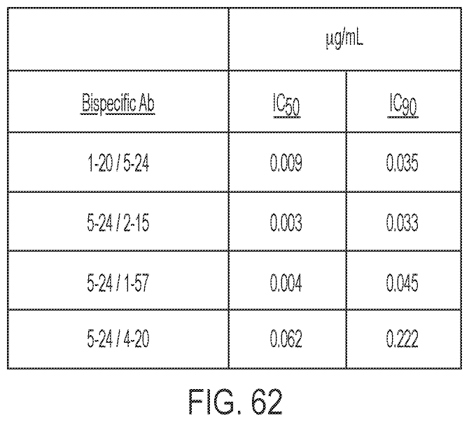

FIG. 62 shows potencies of bispecific antibodies with 5-24 (NTD) as a parental antibody. The IC₅₀ of bispecific antibody 1-20/5-24 is 0.009 μg/ml. The IC₉₀ of bispecific antibody 1-20/5-24 is 0.035 μg/ml. The IC₅₀ of bispecific antibody 5-24/2-15 is 0.003 μg/ml. The IC₉₀ of bispecific antibody 5-24/2-15 is 0.033 μg/ml. The IC₅₀ of bispecific antibody 5-24/1-57 is 0.004 μg/ml. The IC₉₀ of bispecific antibody 5-24/1-57 is 0.045 μg/ml. The IC₅₀ of bispecific antibody 5-24/4-20 is 0.062 μg/ml. The IC₉₀ of bispecific antibody 5-24/4-20 is 0.222 μg/ml.

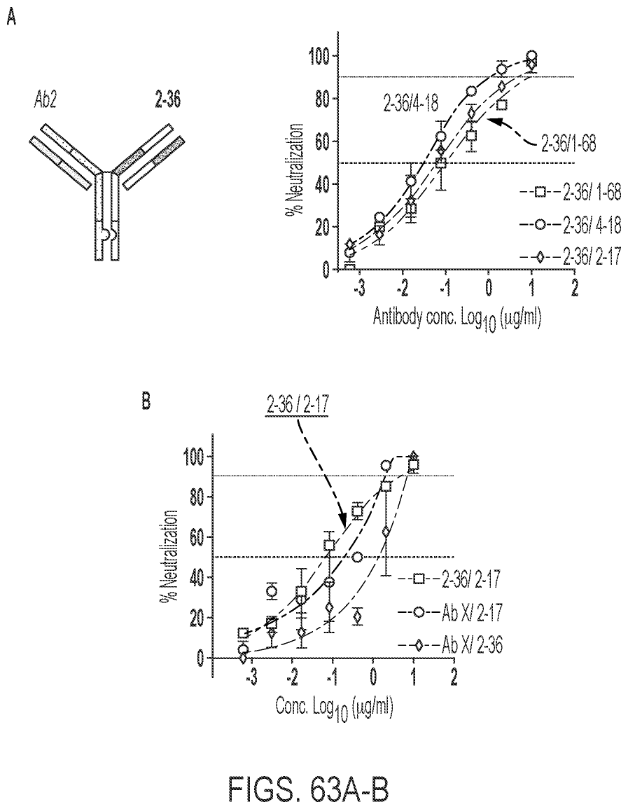

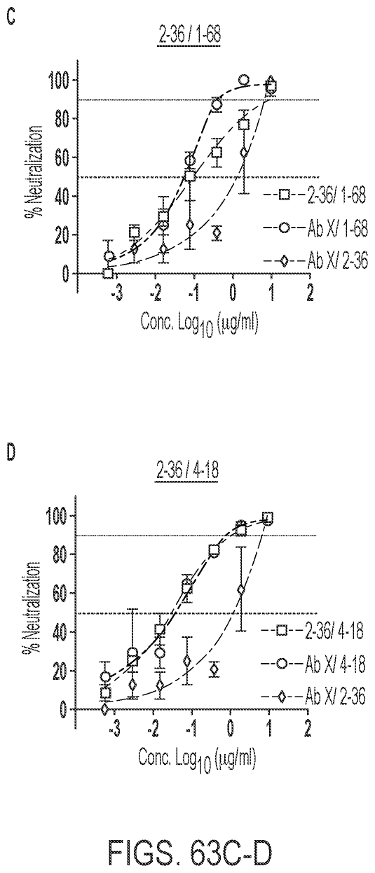

FIGS. 63A-D show bispecific antibodies with 2-36 (RBD) as a parental antibody. FIG. 63A shows virus neutralization with bispecific antibodies 2-36/1-68, 2-36/4-18, and 2-36/2-17. FIG. 63B shows virus neutralization with the bispecific antibody 2-36/2-17 compared to control antibodies. FIG. 63C shows virus neutralization with the bispecific antibody 2-36/1-68 compared to control antibodies. FIG. 63D shows virus neutralization with the bispecific antibody 2-36/4-18 compared to control antibodies.

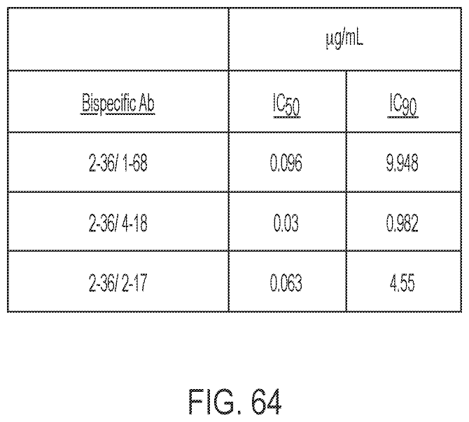

FIG. 64 shows potencies of bispecific antibodies with 2-36 (RBD) as a parental antibody. The IC₅₀ of bispecific antibody 2-36/1-69 is 0.096 μg/ml. The IC₉₀ of bispecific antibody 2-36/1-68 is 9.948 μg/ml. The IC₅₀ of bispecific antibody 2-36/4-18 is 0.03 μg/ml. The IC₉₀ of bispecific antibody 2-36/4-18 is 0.982 μg/ml. The IC₅₀ of bispecific antibody 2-36/2-17 is 0.063 μg/ml. The IC₉₀ of bispecific antibody 2-36/2-17 is 4.55 μg/ml.

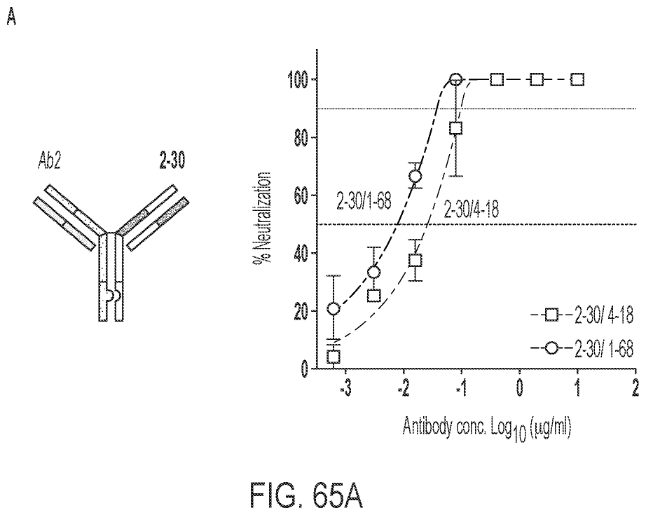

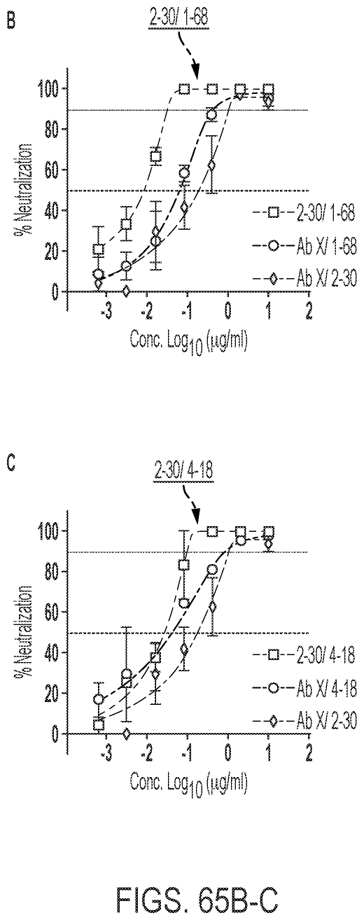

FIGS. 65A-C show bispecific antibodies with 2-30 (RBD) as a parental antibody. FIG. 65A shows virus neutralization with bispecific antibodies 2-30/4-18 and 2-30/1-68. FIG. 65B shows virus neutralization with the bispecific antibody 2-30/1-68 compared to control antibodies. FIG. 65C shows virus neutralization with the bispecific antibody 2-30/4-18 compared to control antibodies.

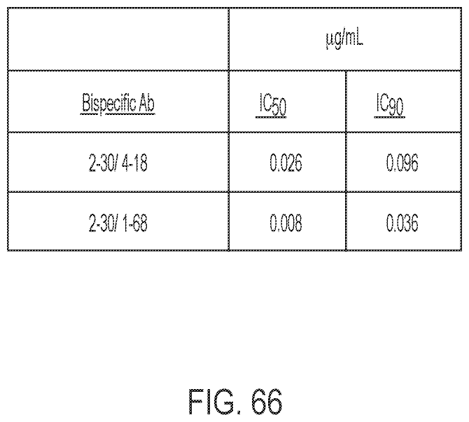

FIG. 66 shows potencies of bispecific antibodies with 2-30 (RBD) as a parental antibody. The IC₅₀ of bispecific antibody 2-30/4-18 is 0.026 μg/ml. The IC₉₀ of bispecific antibody 2-30/4-18 is 0.096 μg/ml. The IC₅₀ of bispecific antibody 2-30/1-68 is 0.008 μg/ml. The IC₉₀ of bispecific antibody 2-30/1-68 is 0.036 μg/ml.

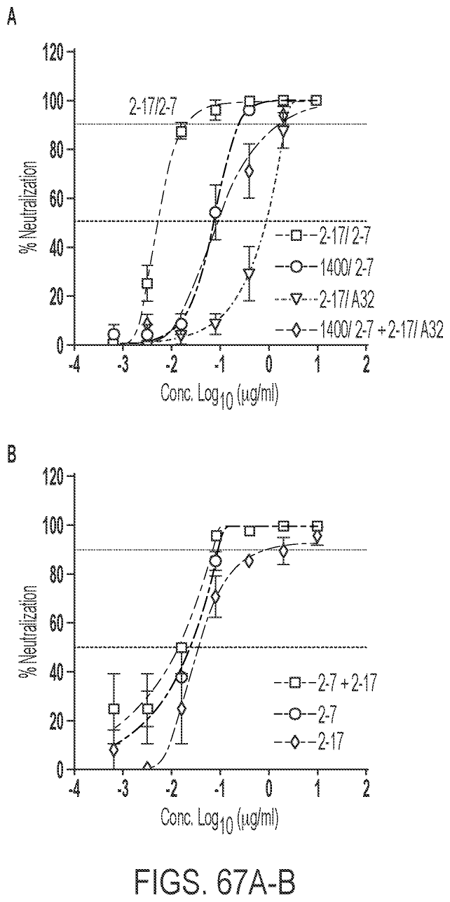

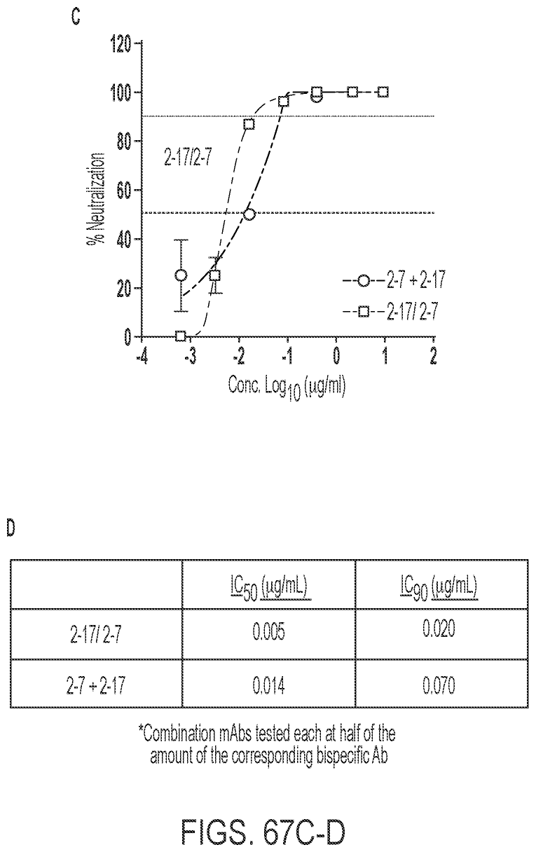

FIGS. 67A-D show comparative evaluations of bispecific antibody 2-17/2-7. FIG. 67A shows the potency of the 2-17/2-7 bispecific antibody compared to an antibody where one arm resembles the individual parental component of the bispecific antibody while the other arm is an irrelevant control. It also compares the potency of the bispecific antibody to the combination of the two single arm IgG controls. FIG. 67B shows virus neutralization with a combination of the parental antibodies and each of the parental antibodies. FIG. 67C shows virus neutralization with the bispecific antibody 2-17/2-7 compared to a combination of the parental antibodies. FIG. 67D shows potency of combination parental monoclonal antibodies tested each at half of the amount of the corresponding bispecific antibody. The IC₅₀ of bispecific antibody 2-17/2-7 is 0.005 μg/ml. The IC₉₀ of bispecific antibody 2-17/2-7 is 0.02 μg/ml. The IC₅₀ of the combination of parental antibodies is 0.014 μg/ml. The IC₉₀ of the combination of parental antibodies is 0.07 μg/ml.

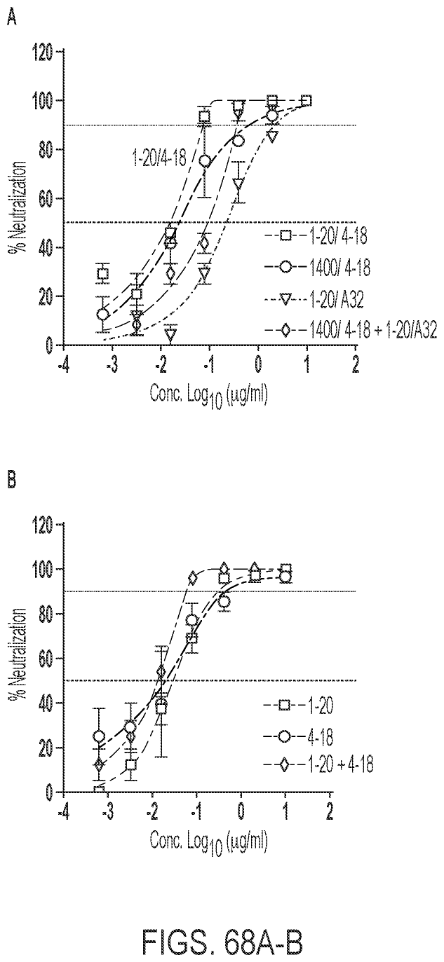

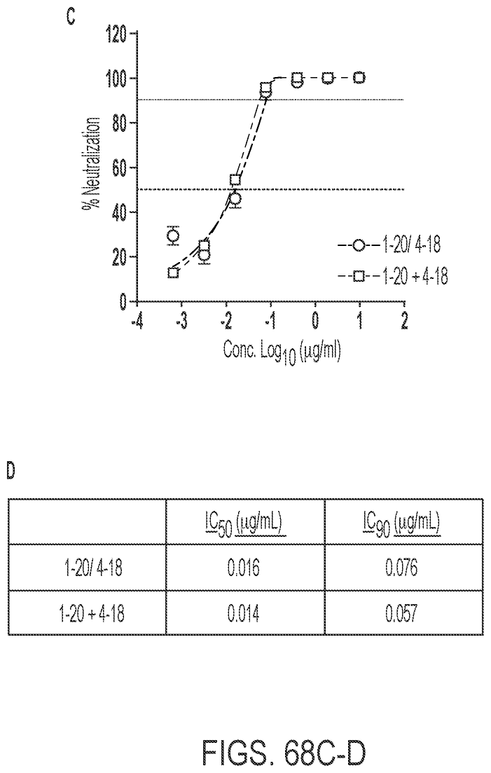

FIGS. 68A-D show comparative evaluations of bispecific antibody 1-20/4-18. FIG. 68A shows the potency of the 1-20/4-18 bispecific antibody compared to an antibody where one arm resembles the individual parental component of the bispecific antibody while the other arm is an irrelevant control. It also compares the potency of the bispecific antibody to the combination of the two single arm IgG controls. FIG. 68B shows virus neutralization with a combination of the parental antibodies and each of the parental antibodies. FIG. 68C shows virus neutralization with the bispecific antibody 1-20/4-18 compared to a combination of the parental antibodies. FIG. 68D shows potency of combination parental monoclonal antibodies tested each at half of the amount of the corresponding bispecific antibody. The IC₅₀ of bispecific antibody 1-20/4-18 is 0.016 μg/ml. The IC₉₀ of bispecific antibody 1-20/4-18 is 0.076 μg/ml. The IC₅₀ of the combination of parental antibodies is 0.014 μg/ml. The IC₉₀ of the combination of parental antibodies is 0.057 μg/ml.

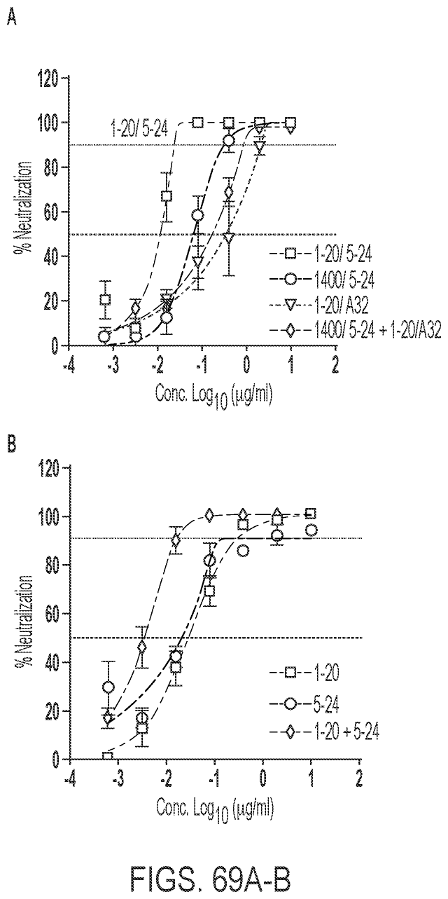

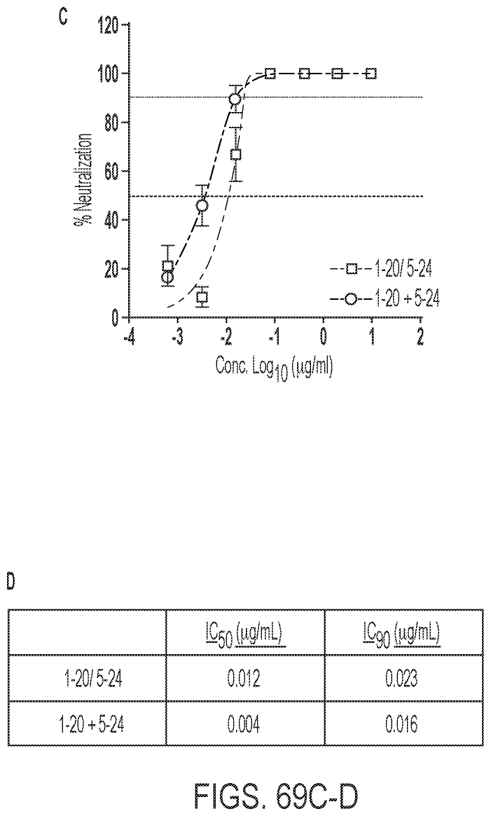

FIGS. 69A-D show comparative evaluations of bispecific antibody 1-20/5-24. FIG. 69A shows the potency of the 1-20/5-24 bispecific antibody compared to an antibody where one arm resembles the individual parental component of the bispecific antibody while the other arm is an irrelevant control. It also compares the potency of the bispecific antibody to the combination of the two single arm IgG controls. FIG. 69B shows virus neutralization with a combination of the parental antibodies and each of the parental antibodies. FIG. 69C shows virus neutralization with the bispecific antibody 1-20/5-24 compared to a combination of the parental antibodies. FIG. 69D shows potency of combination parental monoclonal antibodies tested each at half of the amount of the corresponding bispecific antibody. The IC₅₀ of bispecific antibody 1-20/5-24 is 0.012 μg/ml. The IC₉₀ of bispecific antibody 1-20/5-24 is 0.023 μg/ml. The IC₅₀ of the combination of parental antibodies is 0.004 μg/ml. The IC₉₀ of the combination of parental antibodies is 0.016 μg/ml.

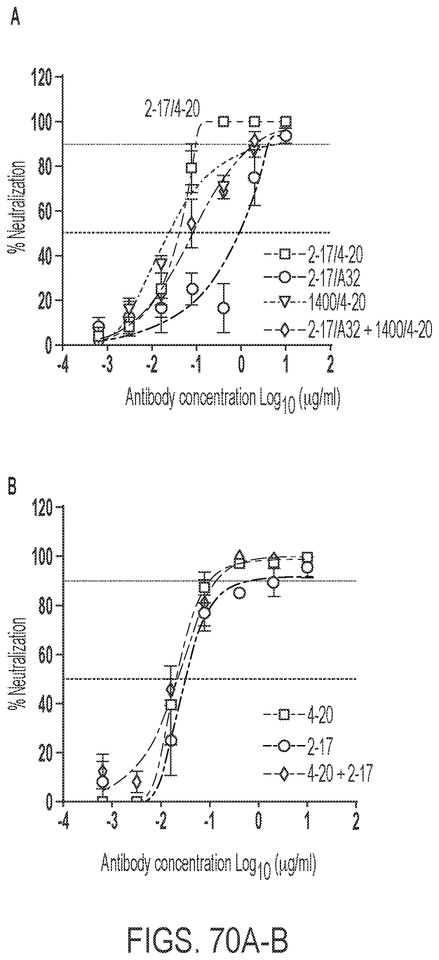

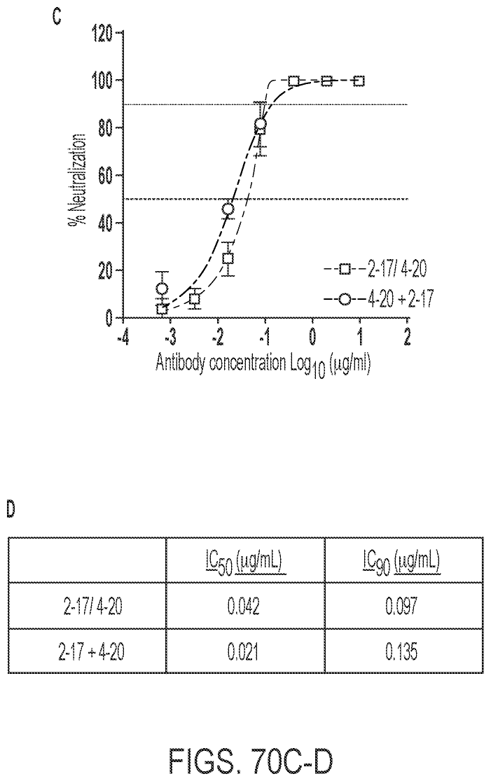

FIGS. 70A-D show comparative evaluations of bispecific antibody 2-17/4-20. FIG. 70A shows the potency of the 2-17/4-20 bispecific antibody to an antibody where one arm resembles the individual parental component of the bispecific antibody while the other arm is an irrelevant control. It also compares the potency of the bispecific antibody to the combination of the two single arm IgG controls. FIG. 70B shows virus neutralization with a combination of the parental antibodies and each of the parental antibodies. FIG. 70C shows virus neutralization with the bispecific antibody 2-17/4-20 compared to a combination of the parental antibodies. FIG. 70D shows potency of combination parental monoclonal antibodies tested each at half of the amount of the corresponding bispecific antibody. The IC₅₀ of bispecific antibody 2-17/4-20 is 0.042 μg/ml. The IC₉₀ of bispecific antibody 2-17/4-20 is 0.097 μg/ml. The IC₅₀ of the combination of parental antibodies is 0.021 μg/ml. The IC₉₀ of the combination of parental antibodies is 0.135 μg/ml.

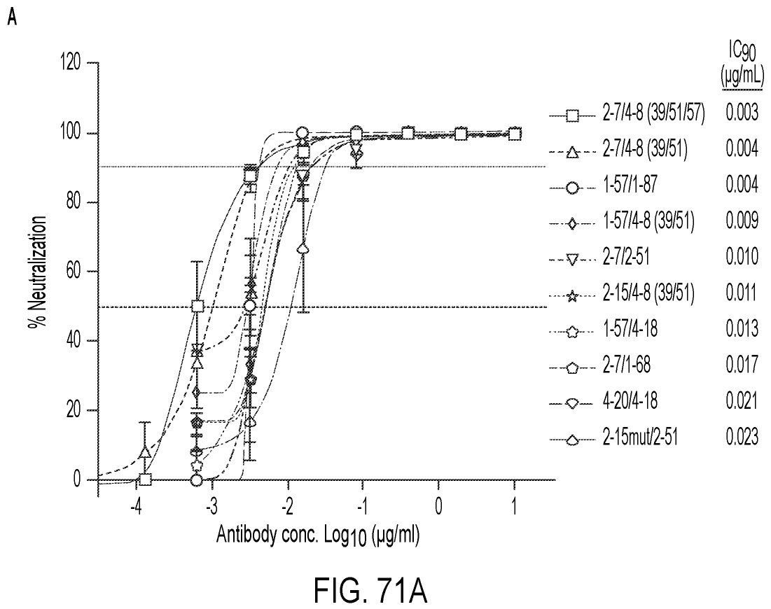

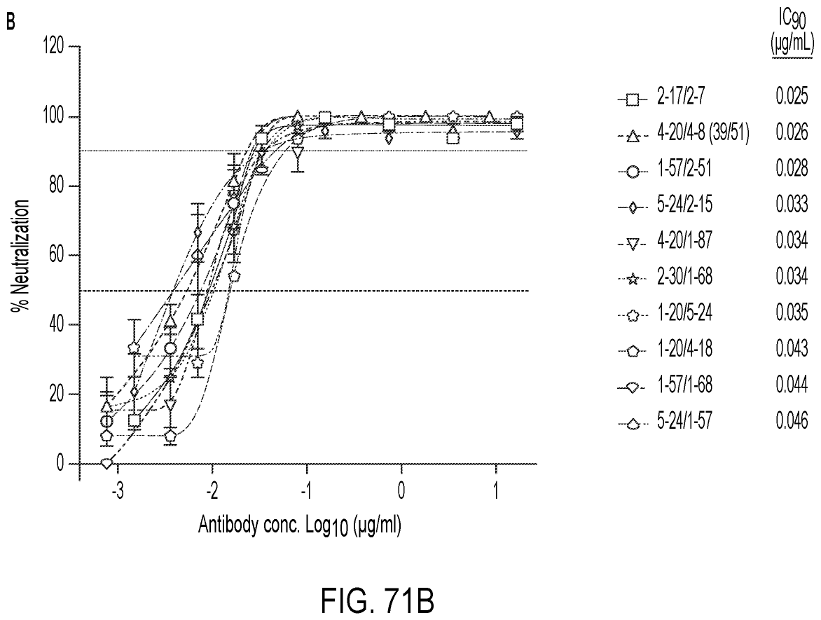

FIGS. 71A-B show in vitro potency of bispecific antibodies against SARS-CoV-2 authentic virus, strain USA/WA1. FIG. 71A shows neutralization for ten bispecific antibodies with IC₉₀ concentrations between 0.003 μg/ml and 0.023 μg/ml. FIG. 71B shows neutralization for nine bispecific antibodies with IC₉₀ concentrations between 0.025 μg/ml and 0.046 μg/ml.

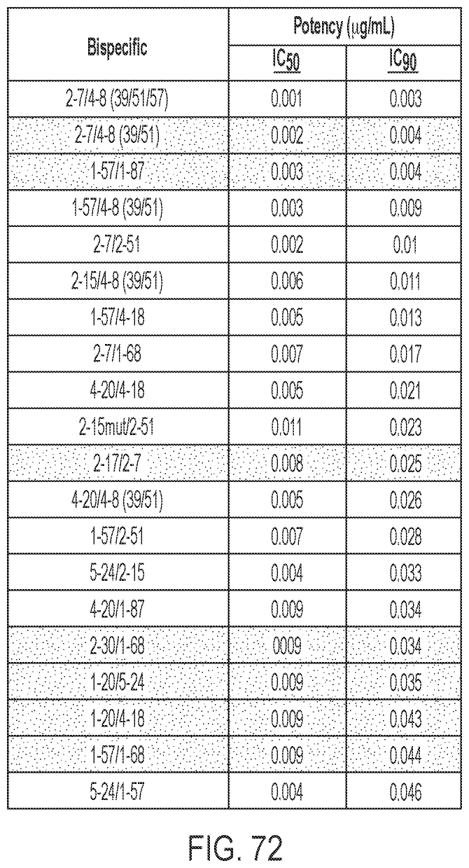

FIG. 72 shows in vitro potency of bispecific antibodies as indicated by respective IC₅₀ and IC₉₀ concentrations.

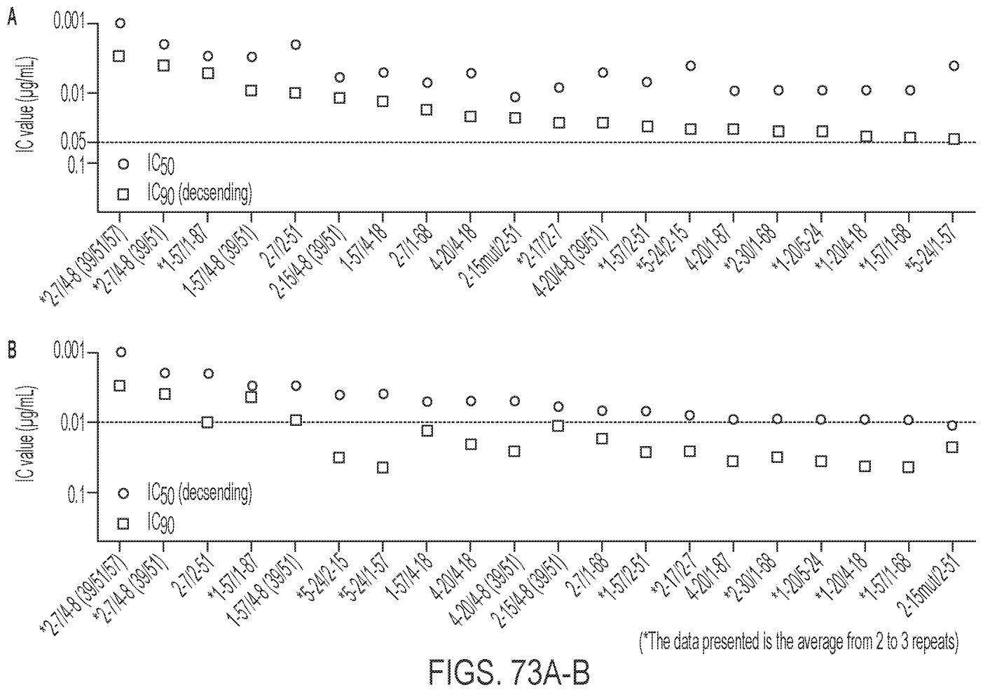

FIGS. 73A-B show in vitro potency of bispecific antibodies showing IC₉₀ and IC₅₀ concentrations. FIG. 73A shows in vitro potency of bispecific antibodies showing IC₉₀ and IC₅₀ concentrations relative to a 0.05 μg/ml threshold. FIG. 73B shows in vitro potency of bispecific antibodies showing IC₉₀ and IC₅₀ concentrations relative to a 0.01 μg/ml threshold. FIGS. 73A and 73B are two separated “SORTED” data. In A, the data is sorted by the top 20 bispecific antibody having the highest to lowest inhibition against 90% of the viruses (IC₉₀). In B, the data is sorted by the top 20 bispecific antibody having the highest to lowest inhibition against 50% of the viruses (IC₅₀).

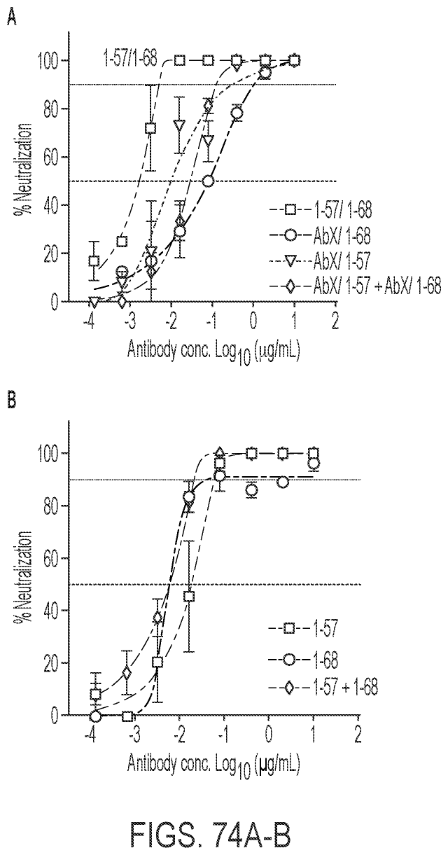

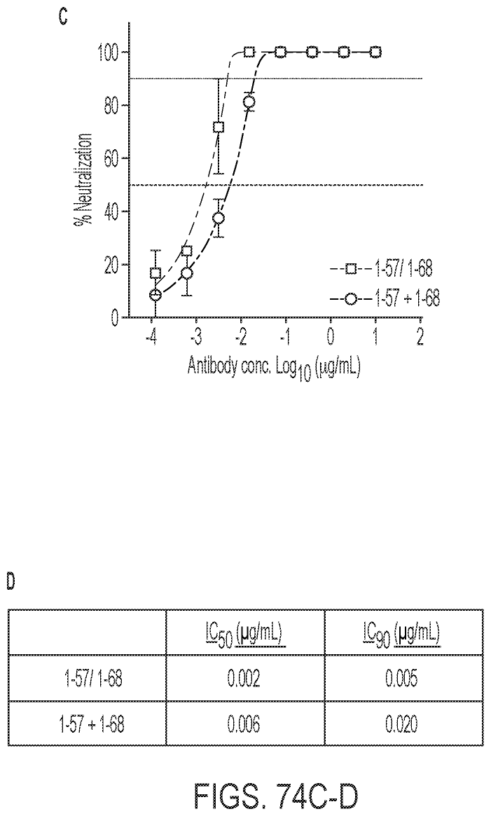

FIGS. 74A-D show comparative evaluation of the bispecific antibody 1-57/1-68. FIG. 74A shows virus neutralization with the bispecific antibody 1-57/1-68 compared to two control bispecific antibodies, each comprising a parental antibody arm and a control antibody arm, and compared to a mix of the two control bispecific antibodies. AbX is an irrelevant control antibody arm, wherein AbX is PGDM1400. FIG. 74B shows virus neutralization with a combination of the parental antibodies and each of the parental antibodies. FIG. 74C shows virus neutralization with the bispecific antibody 1-57/1-68 compared to a combination of the parental antibodies. FIG. 74D shows potency of the bispecific antibody 1-57/1-68 and a mix of each parental monoclonal antibodies tested each at half of the amount of the corresponding bispecific antibody. The IC₅₀ of bispecific antibody 1-57/1-68 is 0.002 μg/ml. The IC₉₀ of bispecific antibody 1-57/1-68 is 0.005 μg/ml. The IC₅₀ of the combination of parental antibodies is 0.006 μg/ml. The IC₉₀ of the combination of parental antibodies is 0.02 μg/ml. The comparative data are from a single experiment, while, the data in the summary table are averaged from multiple repeats.

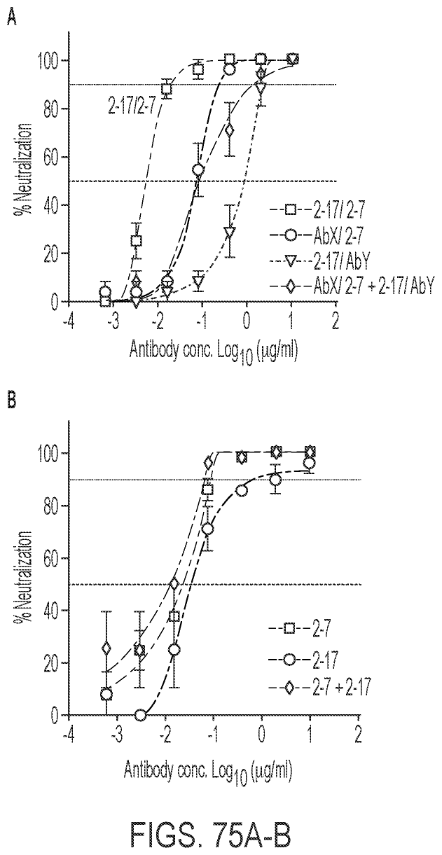

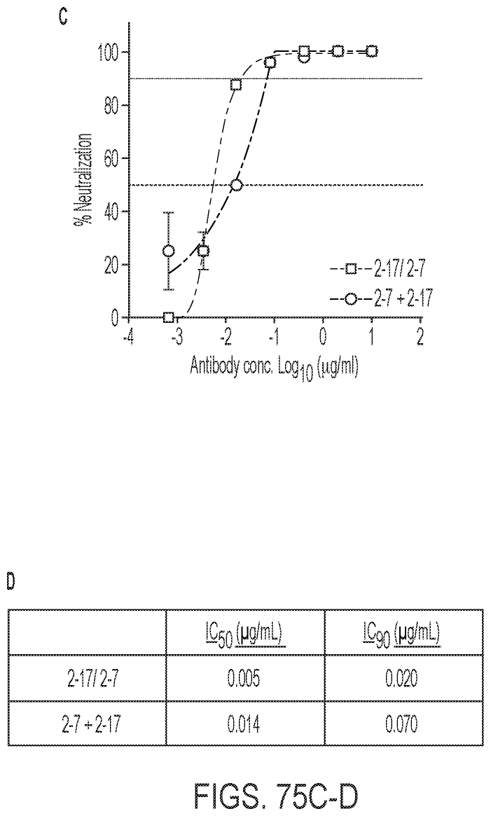

FIGS. 75A-D show comparative evaluation of the bispecific antibody 2-17/2-7. FIG. 75A shows virus neutralization with the bispecific antibody 2-17/2-7 compared to two control bispecific antibodies, each comprising a parental antibody arm and a control antibody arm, and compared to a mix of the two control bispecific antibodies. AbX and AbY are irrelevant control antibody arms, wherein AbX is PGDM1400 and AbY is A32. FIG. 75B shows virus neutralization with a combination of the parental antibodies and each of the parental antibodies. FIG. 75C shows virus neutralization with the bispecific antibody 2-17/2-7 compared to a combination of the parental antibodies. FIG. 75D shows potency of the bispecific antibody 2-17/2-7 and a mix of each parental monoclonal antibodies tested each at half of the amount of the corresponding bispecific antibody. The IC₅₀ of bispecific antibody 2-17/2-7 is 0.005 μg/ml. The IC₉₀ of bispecific antibody 2-17/2-7 is 0.02 μg/ml. The IC₅₀ of the combination of parental antibodies is 0.014 μg/ml. The IC₉₀ of the combination of parental antibodies is 0.07 μg/ml. The comparative data are from a single experiment, while, the data in the summary table are averaged from multiple repeats.

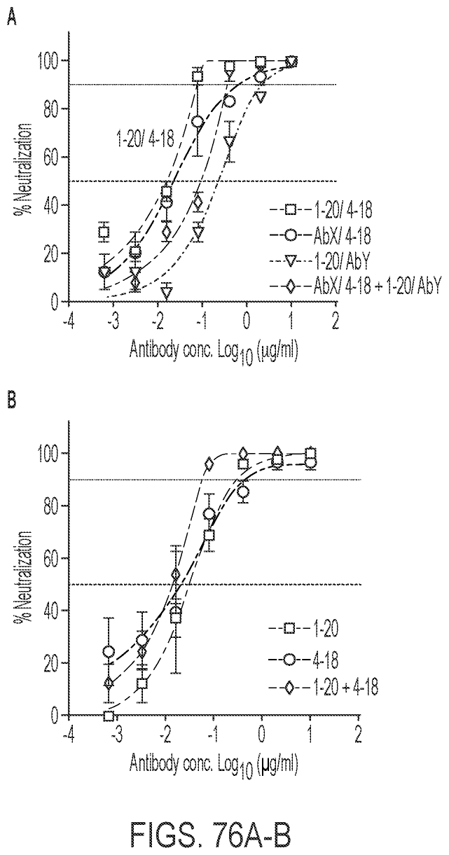

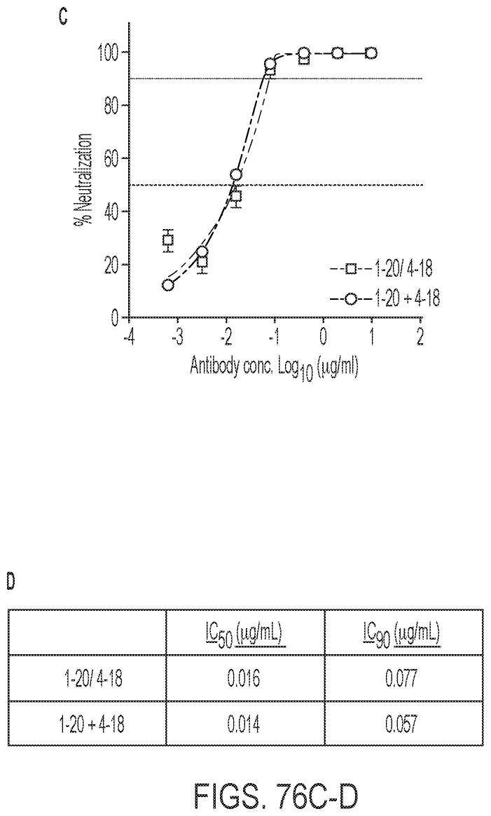

FIGS. 76A-D show comparative evaluation of the bispecific antibody 1-20/4-18. FIG. 76A shows virus neutralization with the bispecific antibody 1-20/4-18 compared to two control bispecific antibodies, each comprising a parental antibody arm and a control antibody arm, and compared to a mix of the two control bispecific antibodies. AbX and AbY are irrelevant control antibody arms, wherein AbX is PGDM1400 and AbY is A32. FIG. 76B shows virus neutralization with a combination of the parental antibodies and each of the parental antibodies. FIG. 76C shows virus neutralization with the bispecific antibody 1-20/4-18 compared to a combination of the parental antibodies. FIG. 76D shows potency of the bispecific antibody 1-20/4-18 and a mix of each parental monoclonal antibodies tested each at half of the amount of the corresponding bispecific antibody. The IC₅₀ of bispecific antibody 1-20/4-18 is 0.016 μg/ml. The IC₉₀ of bispecific antibody 1-20/4-18 is 0.077 μg/ml. The IC₅₀ of the combination of parental antibodies is 0.014 μg/ml. The IC₉₀ of the combination of the parental antibodies is 0.057 μg/ml. The comparative data are from a single experiment, while, the data in the summary table are averaged from multiple repeats.

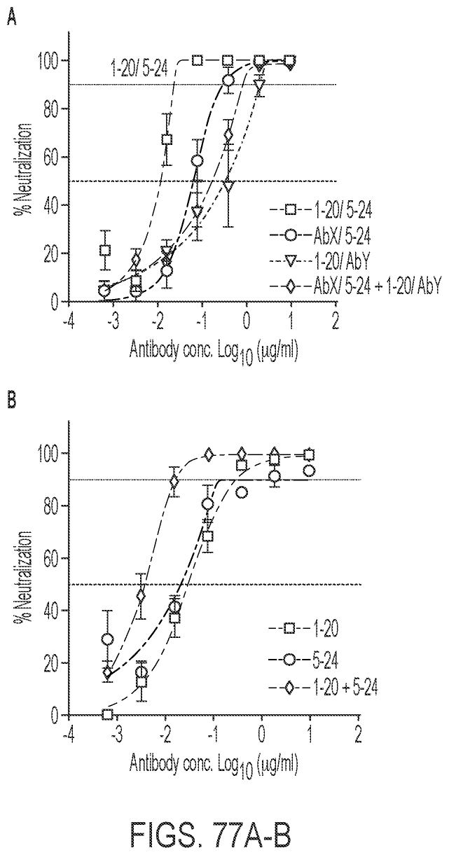

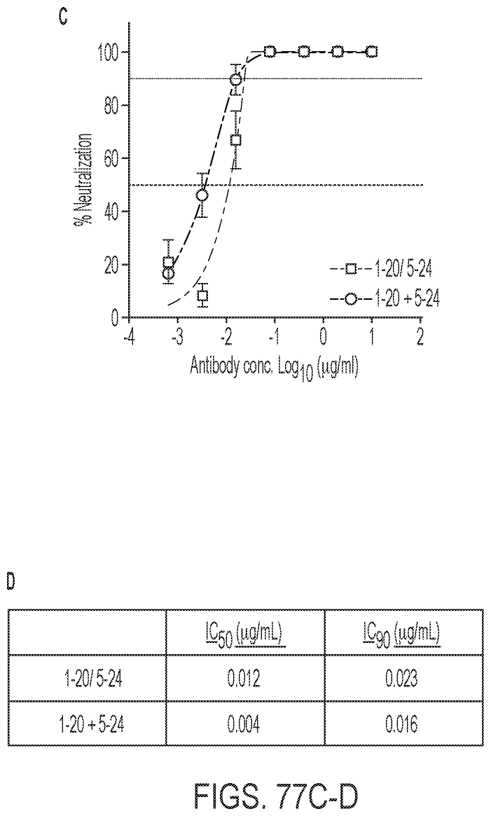

FIGS. 77A-D show comparative evaluation of the bispecific antibody 1-20/5-24. FIG. 77A shows virus neutralization with the bispecific antibody 1-20/5-24 compared to two control bispecific antibodies, each comprising a parental antibody arm and a control antibody arm, and compared to a mix of the two control bispecific antibodies. AbX and AbY are irrelevant control antibody arms, wherein AbX is PGDM1400 and AbY is A32. FIG. 77B shows virus neutralization with a combination of the parental antibodies and each of the parental antibodies. FIG. 77C shows virus neutralization with the bispecific antibody 1-20/5-24 compared to a combination of the parental antibodies. FIG. 77D shows potency of the bispecific antibody 1-20/5-24 and a mix of each parental monoclonal antibodies tested each at half of the amount of the corresponding bispecific antibody. The IC₅₀ of bispecific antibody 1-20/5-24 is 0.012 μg/ml. The IC₉₀ of bispecific antibody 1-20/5-24 is 0.023 μg/ml. The IC₅₀ of the combination of parental antibodies is 0.004 μg/ml. The IC₉₀ of the combination of parental antibodies is 0.016 μg/ml. The comparative data are from a single experiment, while, the data in the summary table are averaged from multiple repeats.

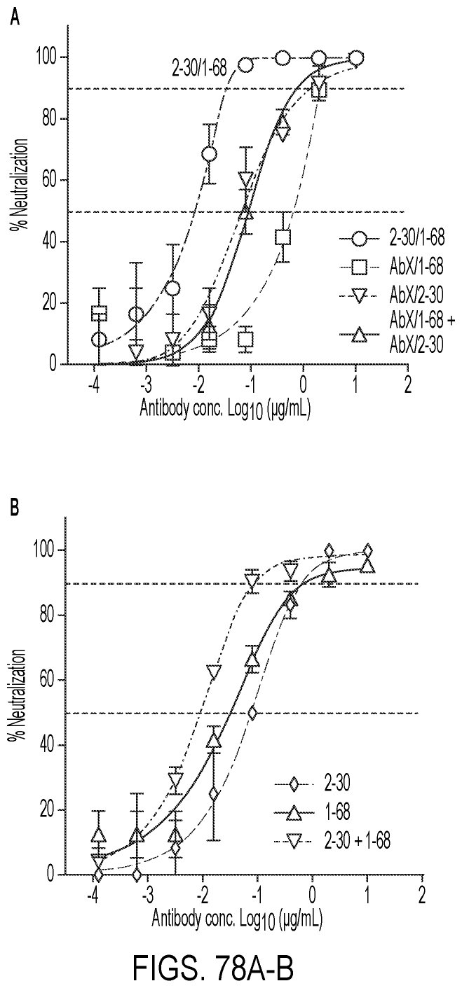

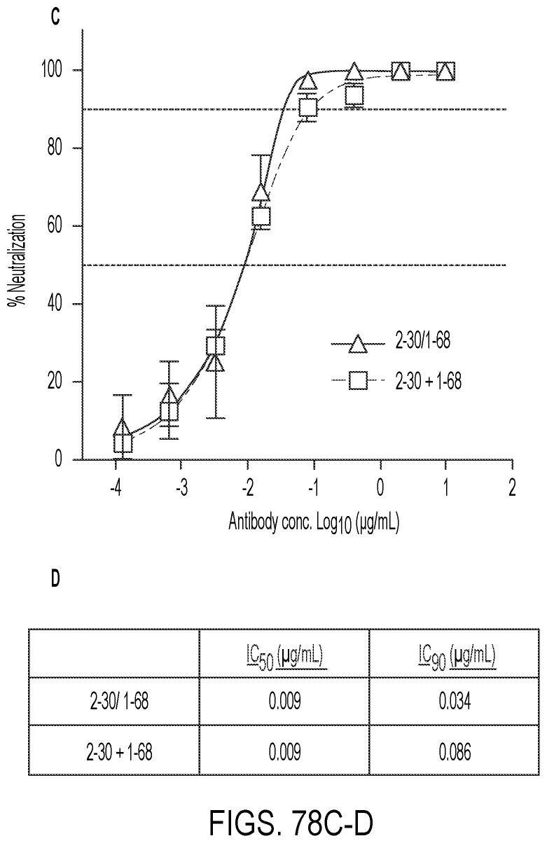

FIGS. 78A-D show comparative evaluation of the bispecific antibody 2-30/1-68. FIG. 78A shows virus neutralization with the bispecific antibody 2-30/1-68 compared to two control bispecific antibodies, each comprising a parental antibody arm and a control antibody arm, and compared to a mix of the two control bispecific antibodies. AbX is an irrelevant control antibody arms, wherein AbX is PGDM1400. FIG. 78B shows virus neutralization with a combination of the parental antibodies and each of the parental antibodies. FIG. 78C shows virus neutralization with the bispecific antibody 2-30/1-68 compared to a combination of the parental antibodies. FIG. 78D shows potency of the bispecific antibody 2-30/1-68 and a mix of each parental monoclonal antibodies tested each at half of the amount of the corresponding bispecific antibody. The IC₅₀ of bispecific antibody 2-30/1-68 is 0.009 μg/ml. The IC₉₀ of bispecific antibody 2-30/1-68 is 0.034 μg/ml. The IC₅₀ of the combination of parental antibodies is 0.009 μg/ml. The IC₉₀ of the combination of parental antibodies is 0.086 μg/ml. The comparative data are from a single experiment, while, the data in the summary table are averaged from multiple repeats.

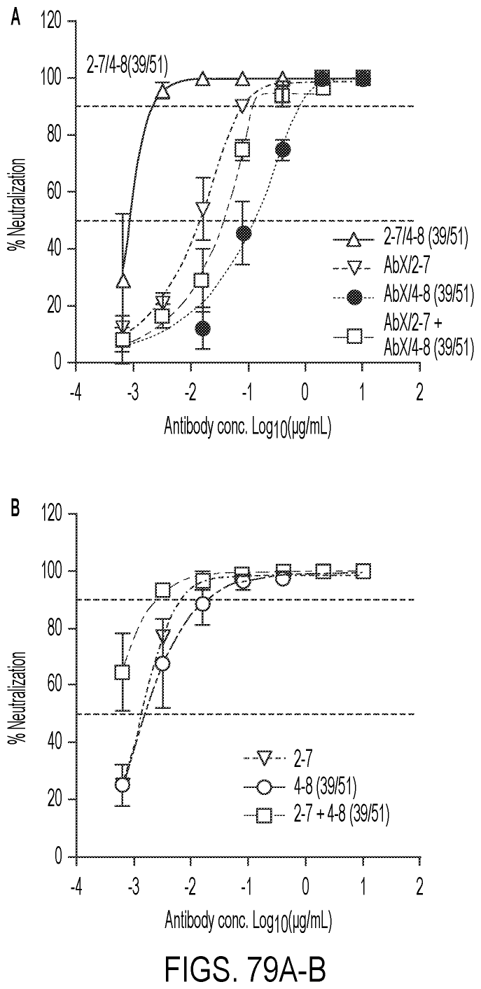

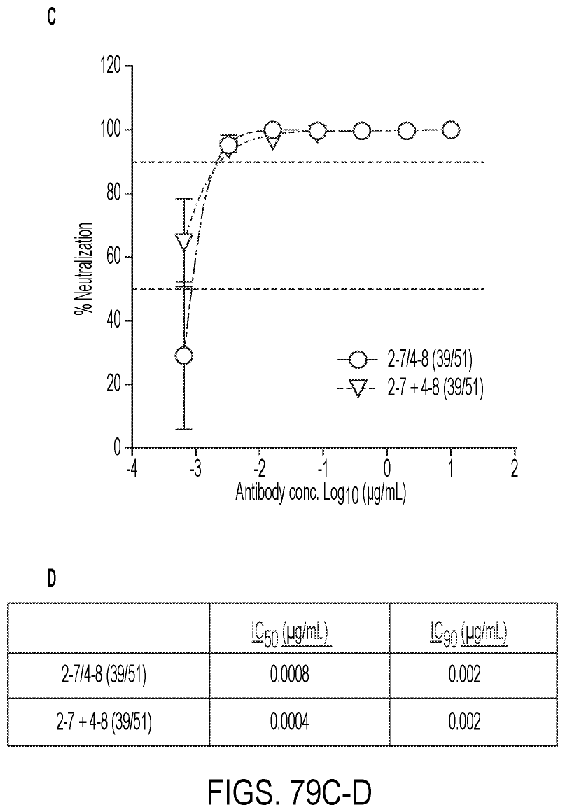

FIGS. 79A-D show comparative evaluation of the bispecific antibody 2-7/4-8 (39/51). FIG. 79A shows virus neutralization with the bispecific antibody 2-7/4-8 (39/51) compared to two control bispecific antibodies, each comprising a parental antibody arm and a control antibody arm, and compared to a mix of the two control bispecific antibodies. AbX is an irrelevant control antibody arms, wherein AbX is PGDM1400. FIG. 79B shows virus neutralization with a combination of the parental antibodies and each of the parental antibodies. FIG. 79C shows virus neutralization with the bispecific antibody 2-7/4-8 (39/51) compared to a combination of the parental antibodies. FIG. 79D shows potency of the bispecific antibody 2-7/4-8 (39/51) and a mix of each parental monoclonal antibodies tested each at half of the amount of the corresponding bispecific antibody. The IC₅₀ of bispecific antibody 2-7/4-8(39/51) is 0.0008 μg/ml. The IC₉₀ of bispecific antibody 2-7/4-8(39/51) is 0.002 μg/ml. The comparative data are from a single experiment, while, the data in the summary table are averaged from multiple repeats.

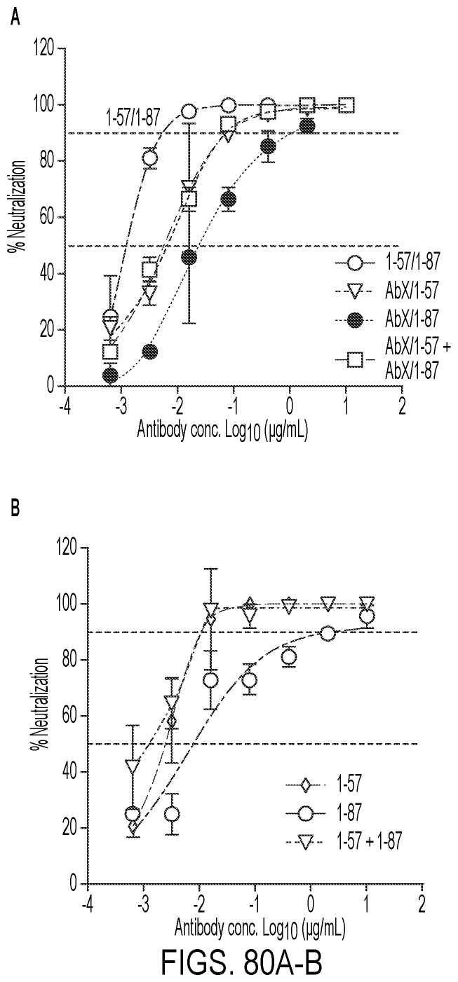

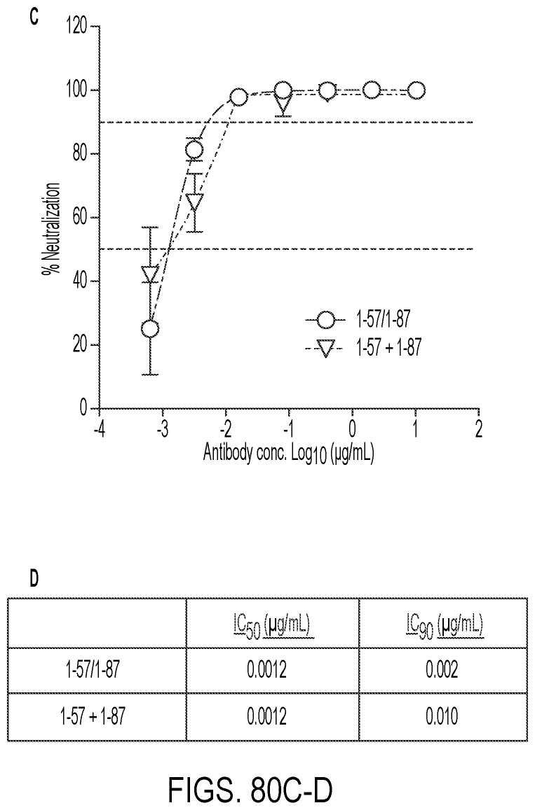

FIGS. 80A-D show comparative evaluation of the bispecific antibody 1-57/1-87. FIG. 80A shows virus neutralization with the bispecific antibody 1-57/1-87 compared to two control bispecific antibodies, each comprising a parental antibody arm and a control antibody arm, and compared to a mix of the two control bispecific antibodies. AbX is an irrelevant control antibody arms, wherein AbX is PGDM1400. FIG. 80B shows virus neutralization with a combination of the parental antibodies and each of the parental antibodies. FIG. 80C shows virus neutralization with the bispecific antibody 1-57/1-87 compared to a combination of the parental antibodies. FIG. 80D shows potency of the bispecific antibody 1-57/1-87 and a mix of each parental monoclonal antibodies tested each at half of the amount of the corresponding bispecific antibody. The IC₅₀ of bispecific antibody 1-57/1-87 is 0.0012 μg/ml. The IC₉₀ of bispecific antibody 1-57/1-87 is 0.002 μg/ml. The IC₅₀ of the combination of parental antibodies is 0.0012 μg/ml. The IC₉₀ of the combination of parental antibodies is 0.010 μg/ml. The comparative data are from a single experiment, while, the data in the summary table are averaged from multiple repeats.

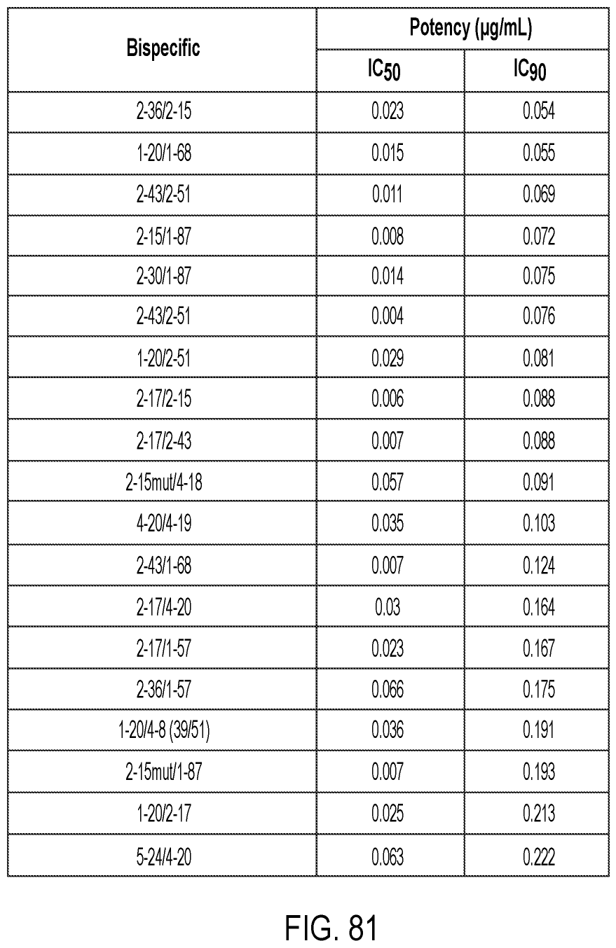

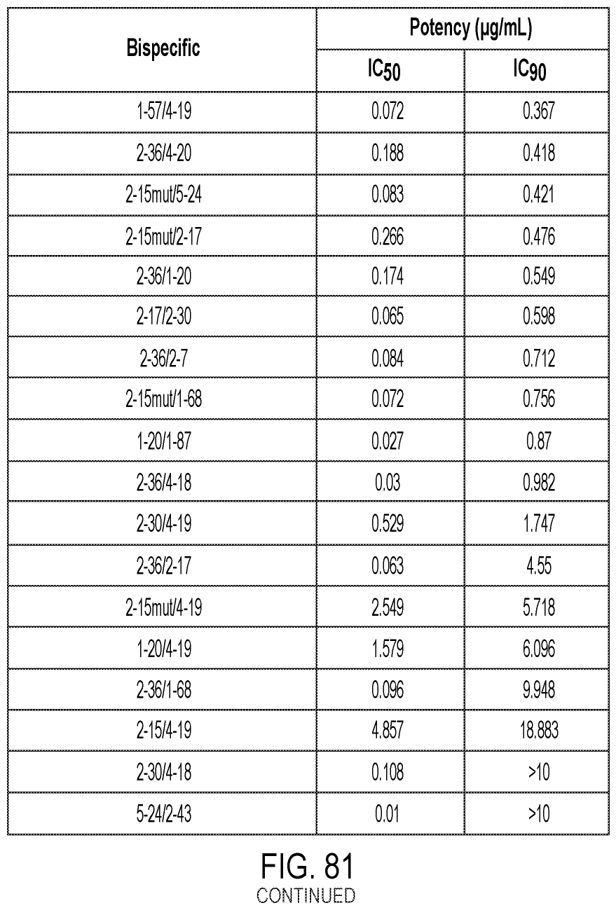

FIG. 81 shows potency of additional bispecific antibodies.

### Example 16—Bispecific Against SARS-CoV-2 Variants

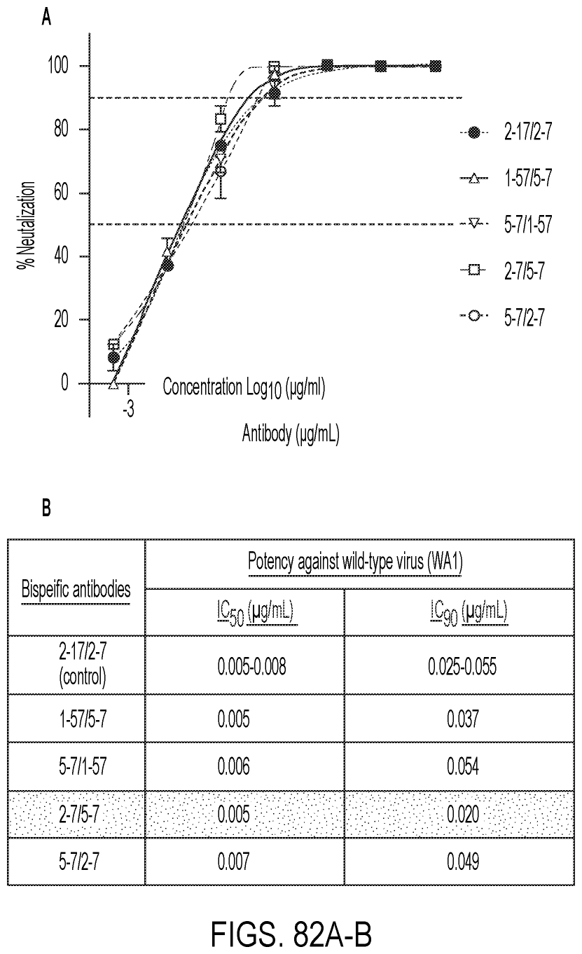

FIGS. 82A-B show neutralizing activities of engineered bispecific antibodies. FIG. 82A shows neutralizing activities against wild-type virus (WA1) at various antibody concentrations for bispecific antibodies 2-17/2-7, 1-57/5-7, 5-7/1-57, 2-7/5-7, 5-7/2-7. FIG. 82B shows antibody potency (IC₅₀ and IC₉₀) against wild-type virus (WA1) for bispecific antibodies 2-17/2-7 (control), 1-57/5-7, 5-7/1-57, 2-7/5-7, 5-7/2-7. The IC₅₀ of bispecific antibody 2-17/2-7 is 0.005˜0.008 μg/ml. The IC₉₀ of bispecific antibody 2-17/2-7 is 0.025˜0.055 μg/ml. The IC₅₀ of bispecific antibody 1-57/5-7 is 0.005 μg/ml. The IC₉₀ of bispecific antibody 1-57/5-7 is 0.037 μg/ml. The IC₅₀ of bispecific antibody 5-7/1-57 is 0.006 μg/ml. The IC₉₀ of bispecific antibody 5-7/1-57 is 0.054 μg/ml. The IC₅₀ of bispecific antibody 2-7/5-7 is 0.005 μg/ml. The IC₉₀ of bispecific antibody 2-7/5-7 is 0.020 μg/ml. The IC₅₀ of bispecific antibody 5-7/2-7 is 0.007 μg/ml. The IC₉₀ of bispecific antibody 5-7/2-7 is 0.049 μg/ml.

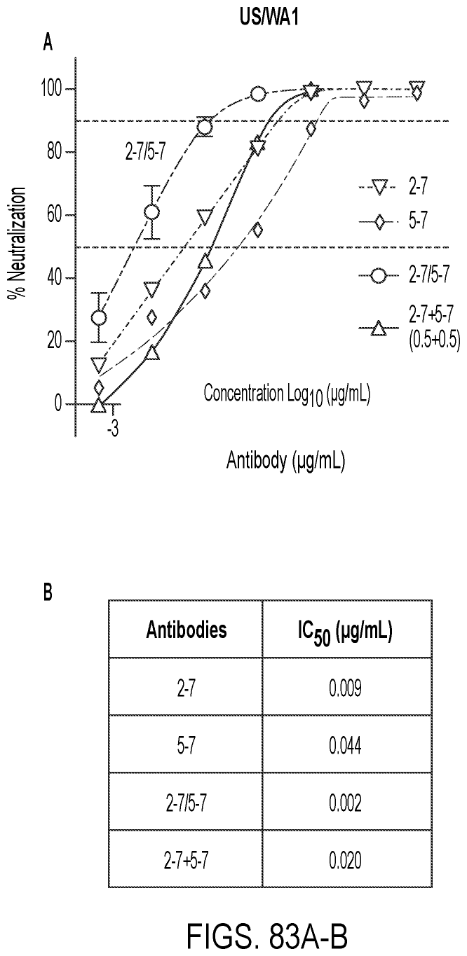

FIGS. 83A-B show 2-7/5-7 bispecific antibody and parental antibodies activities. FIG. 83A shows neutralizing activities against wild-type virus (WA1) at various antibody concentrations for bispecific antibody 2-7/5-7, parental antibodies 2-7, 5-7, and a combination of 2-7 and 5-7 parental antibodies. FIG. 83B shows IC₅₀ concentrations for bispecific antibody 2-7/5-7, parental antibodies 2-7, 5-7, and a combination of 2-7 and 5-7 parental antibodies. The IC₅₀ of parental antibody 2-7 is 0.009 μg/ml. The IC₅₀ of parental antibody 5-7 is 0.044 μg/ml.

The IC₅₀ of bispecific antibody 2-7/5-7 is 0.002 μg/ml. The IC₅₀ of the combination of parental antibodies 2-7 and 5-7 is 0.020 μg/ml.

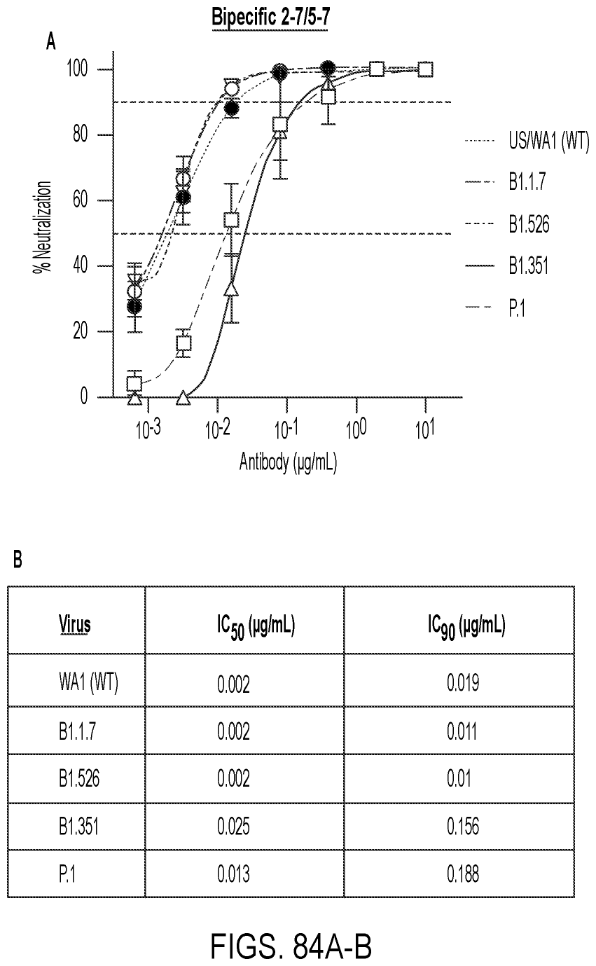

FIGS. 84A-B show activities of bispecific antibody 2-7/5-7 against SARS-CoV-2 wild-type (WA1) and variant strains B1.1.7, B1.526, B1351, and P.1. FIG. 84A shows neutralization activity of bispecific antibody 2-7/5-7 against various SARS-CoV-2 strains at different antibody concentrations. The SARS-CoV-2 strains are WA1, B1.1.7, B1.351, B1.526, and P.1. FIG. 84B shows IC₅₀ and IC₉₀ concentrations for bispecific antibody 2-7/5-7 against various SARS-CoV-2 strains. The IC₅₀ for the bispecific antibody 2-7/5-7 against WA1 (wild-type strain) is 0.002 μg/ml. The IC₉₀ for the bispecific antibody 2-7/5-7 against WA1 (wild-type strain) is 0.019 μg/ml. The IC₅₀ for the bispecific antibody 2-7/5-7 against B1.1.7 is 0.002 μg/ml. The IC₉₀ for the bispecific antibody 2-7/5-7 against B1.1.7 is 0.011 μg/ml. The IC₅₀ for the bispecific antibody 2-7/5-7 against B1.526 is 0.002 μg/ml. The IC₉₀ for the bispecific antibody 2-7/5-7 against B1.526 is 0.01 μg/ml. The IC₅₀ for the bispecific antibody 2-7/5-7 against B1.351 is 0.025 μg/ml. The IC₉₀ for the bispecific antibody 2-7/5-7 against B1.351 is 0.156 μg/ml. The IC₅₀ for the bispecific antibody 2-7/5-7 against P.1 is 0.013 μg/ml. The IC₉₀ for the bispecific antibody 2-7/5-7 against P.1 is 0.188 μg/ml.

### Example 17—Bispecific 2-7/5-7 Antibody

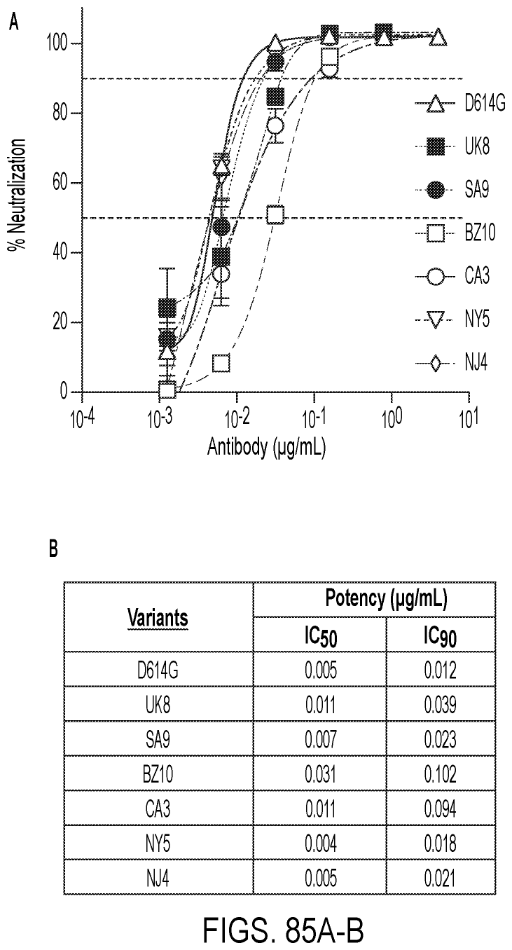

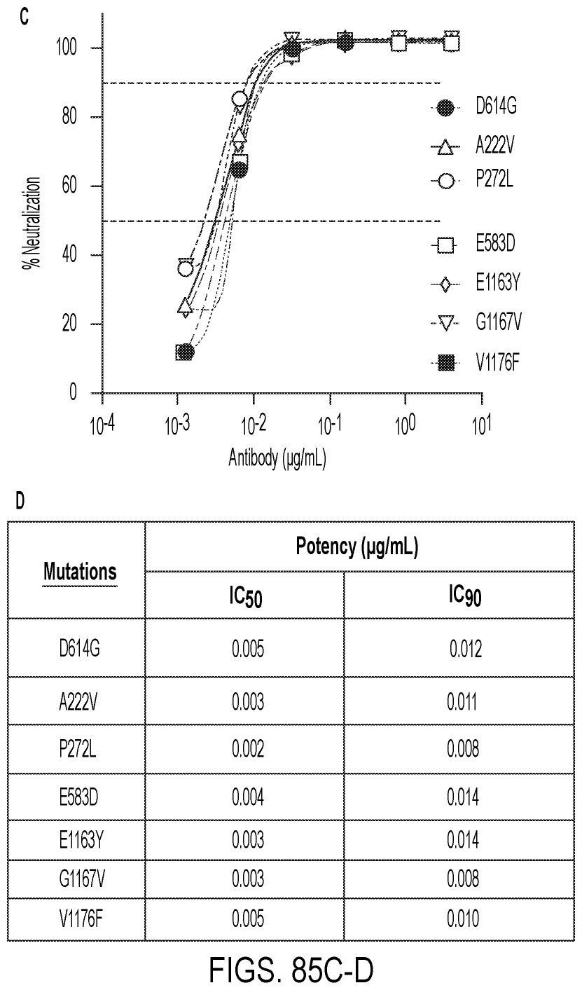

FIGS. 85A-D show neutralization of pseudoviruses having mutations associated with SARS-CoV-2 variants with the bispecific 2-7/5-7 antibody. FIG. 85A shows antibody neutralization curves against circulating SARS-CoV-2 virus variants. FIG. 85B shows antibody potency against circulating SARS-CoV-2 virus variants. FIG. 85C shows antibody neutralization curves against SARS-CoV-2 variants with high frequency mutations. FIG. 85D shows antibody potency against SARS-CoV-2 variants with high frequency mutations.

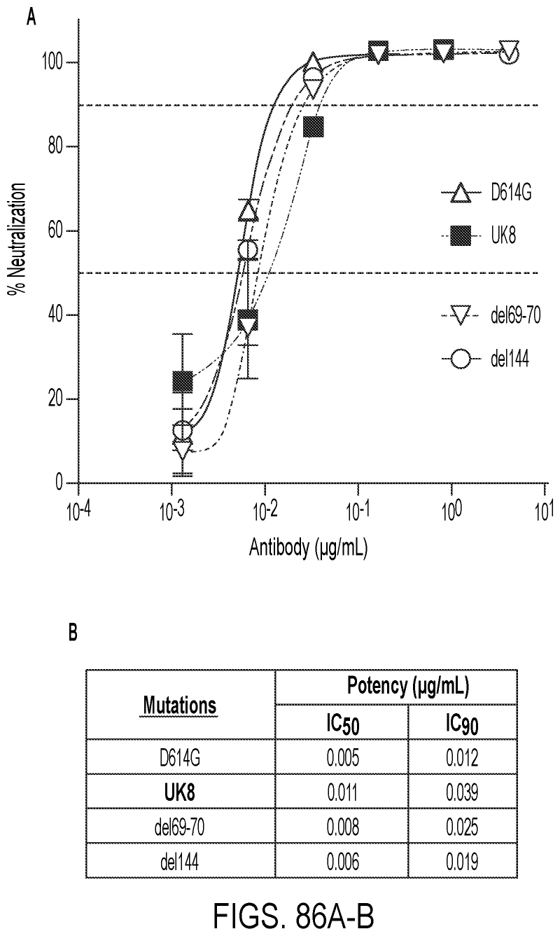

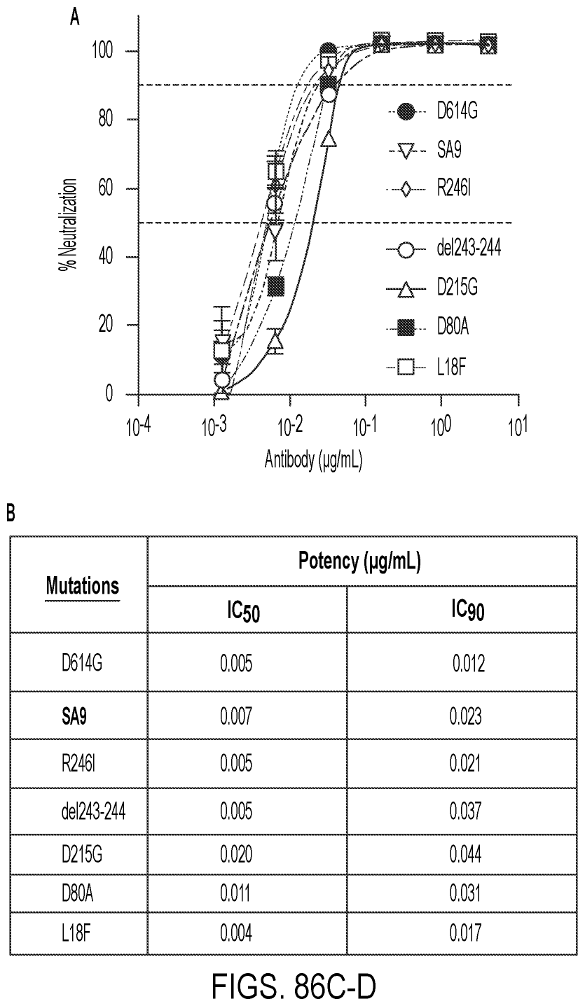

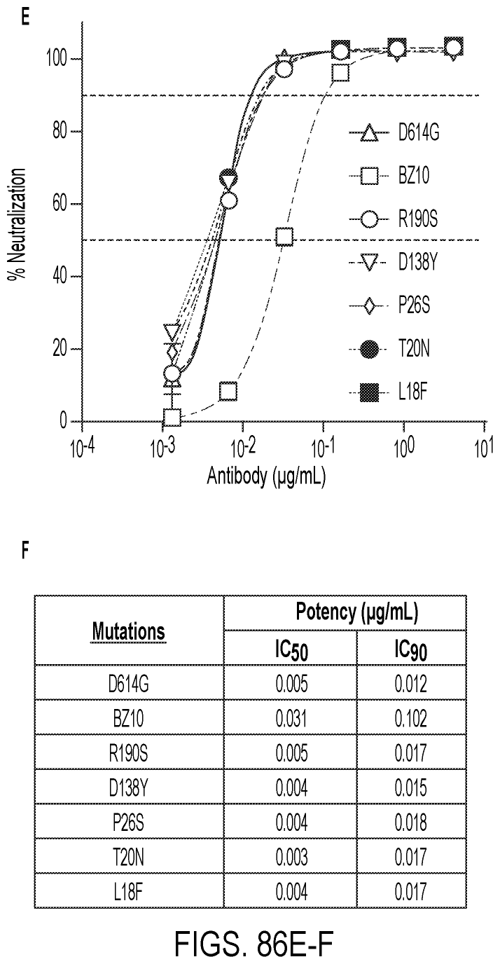

FIGS. 86A-F show neutralization of pseudoviruses with SARS-CoV-2 mutations corresponding to “variants of concern” using the bispecific antibody 2-7/5-7. FIG. 86A shows antibody neutralization curves against B.1.1.7 (UK virus variant) with NTD mutations. FIG. 86B shows antibody potency against B.1.1.7 (UK virus variant) with NTD mutations. FIG. 86C shows antibody neutralization curves against B.1.352 (SA) virus variant with NTD mutations. FIG. 86D shows antibody potency against B.1.352 (SA) virus variant with NTD mutations. FIG. 86E shows antibody neutralization curves against P.1 (BZ) virus variant with NTD mutations. FIG. 86F shows antibody potency against P.1 (BZ) virus variant with NTD mutations.

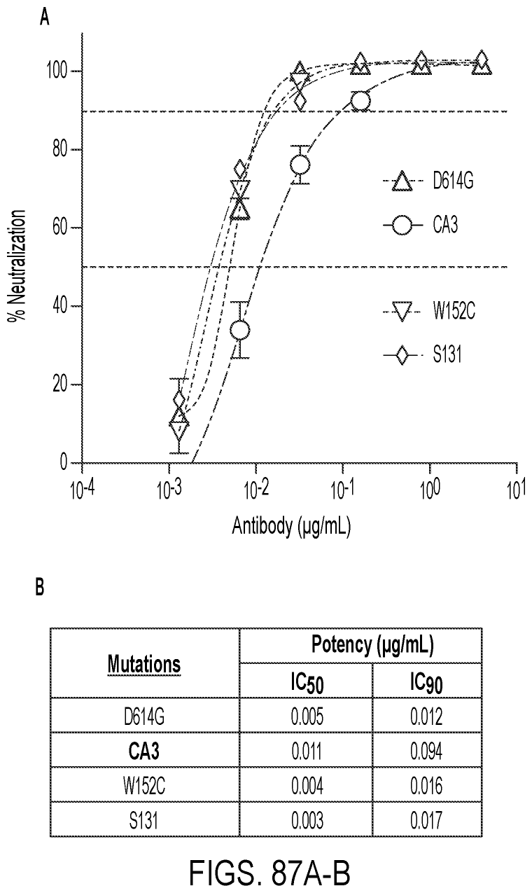

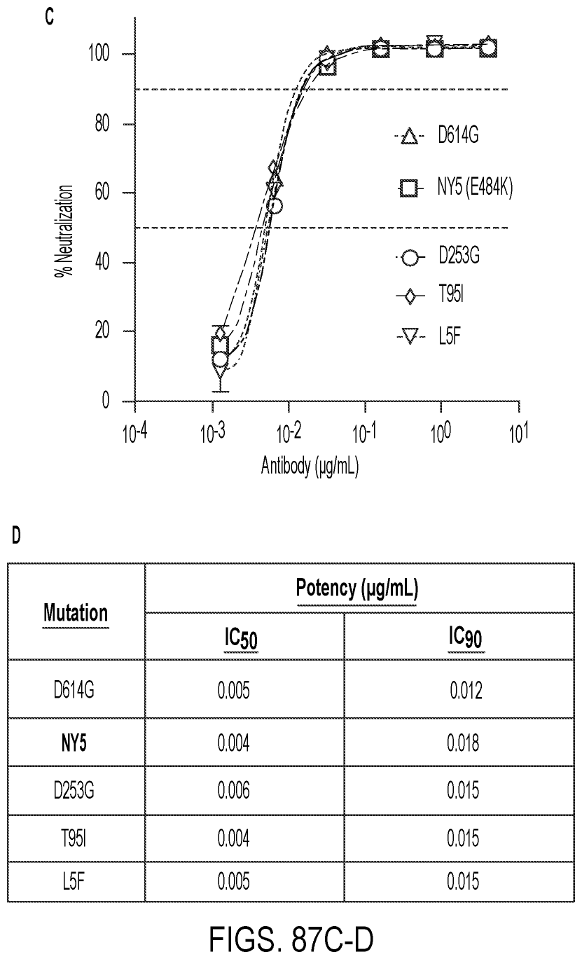

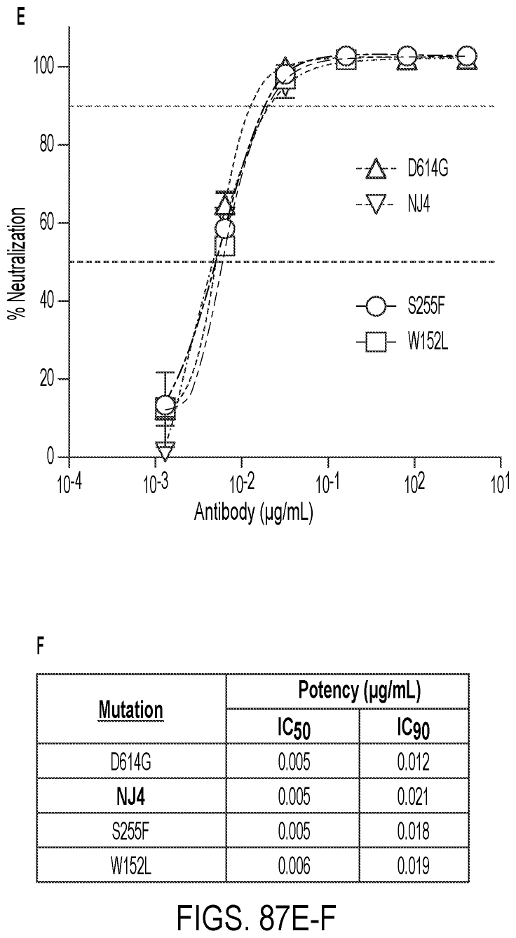

FIGS. 87A-F show neutralization of pseudoviruses with SARS-CoV-2 mutations corresponding to “variants of interest” using the bispecific antibody 2-7/5-7. FIG. 87A shows antibody neutralization curves against B.1.427 (CA) virus variant with NTD mutations. FIG. 87B shows antibody potency against B.1.427 (CA) virus variant with NTD mutations. FIG. 87C shows antibody neutralization curves against B.1.526 (NY) variant with NTD mutations. FIG. 87D shows antibody potency against B.1.526 (NY) variant with NTD mutations. FIG. 87E show antibody neuralization curves against NJ variant with NTD mutations. FIG. 87F show antibody potency against NJ variant with NTD mutations.

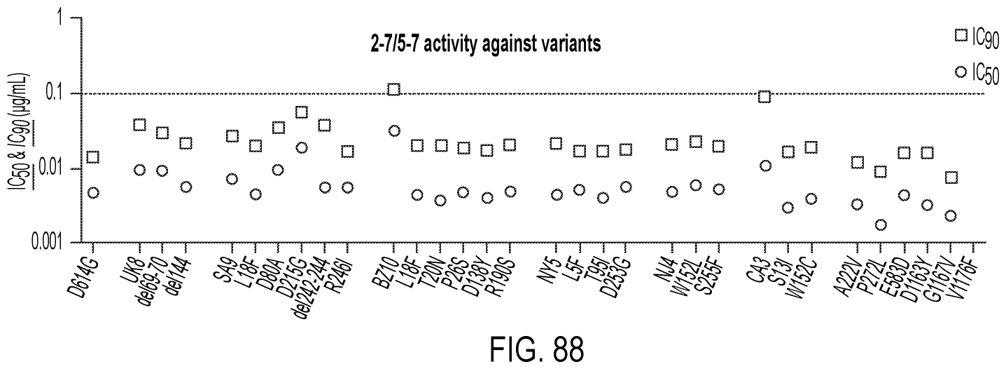

FIG. 88 shows summary of bispecific antibody 2-7/5-7 activity against variants. 1050 is shown in circles. 1C90 is shown in squares.

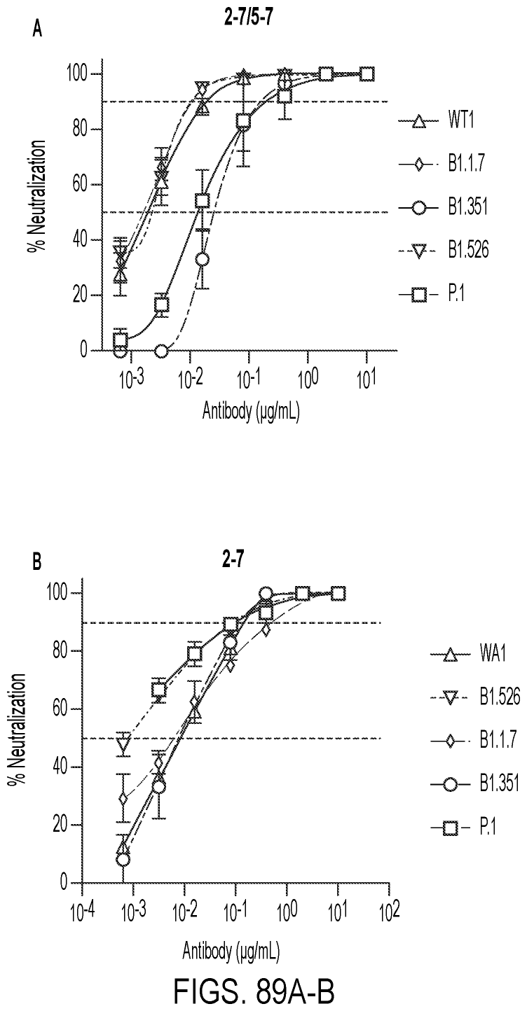

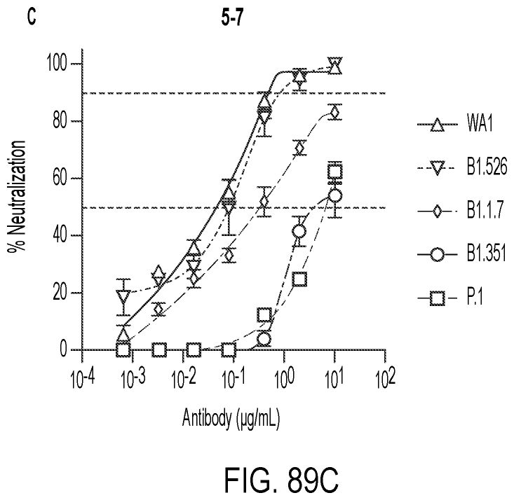

FIGS. 89A-C shows neutralization activity of bispecific antibody 2-7/5-7 and parental 2-7 and 5-7 monoclonal antibodies against SARS-CoV-2 variants (live viruses). FIG. 89A shows the neutralization curve for bispecific antibody 2-7/5-7. FIG. 89B shows the neutralization curve for parental antibody 2-7. FIG. 89C shows the neutralization curve for parental antibody 5-7.

1. A synthesized monoclonal antibody, or a functional fragment thereof, comprising a heavy chain having a variable domain and a constant domain and a light chain having a variable domain and a constant domain, wherein the antibody binds an antigen on a coronavirus particle.

2-7. (canceled)

8. The antibody of claim 1, wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 29 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 30;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 31 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 32;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 33 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 34;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 39 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 40;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44; or
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 45 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 46.

9-22. (canceled)

23. The antibody of claim 1, wherein the heavy chain variable domain comprises SEQ ID NO: 3 and the light chain variable domain comprises SEQ ID NO:
wherein the heavy chain variable domain comprises SEQ ID NO: 7 and the light chain variable domain comprises SEQ ID NO: 8;
wherein the heavy chain variable domain comprises SEQ ID NO: 9 and the light chain variable domain comprises SEQ ID NO: 10;
wherein the heavy chain variable domain comprises SEQ ID NO: 11 and the light chain variable domain comprises SEQ ID NO: 12;
wherein the heavy chain variable domain comprises SEQ ID NO: 17 and the light chain variable domain comprises SEQ ID NO: 18;
wherein the heavy chain variable domain comprises SEQ ID NO: 19 and the light chain variable domain comprises SEQ ID NO: 20;
wherein the heavy chain variable domain comprises SEQ ID NO: 21 and the light chain variable domain comprises SEQ ID NO: 22;
wherein the heavy chain variable domain comprises SEQ ID NO: 25 and the light chain variable domain comprises SEQ ID NO: 26;
wherein the heavy chain variable domain comprises SEQ ID NO: 29 and the light chain variable domain comprises SEQ ID NO: 30;
wherein the heavy chain variable domain comprises SEQ ID NO: 31 and the light chain variable domain comprises SEQ ID NO: 32;
wherein the heavy chain variable domain comprises SEQ ID NO: 33 and the light chain variable domain comprises SEQ ID NO: 34;
wherein the heavy chain variable domain comprises SEQ ID NO: 37 and the light chain variable domain comprises SEQ ID NO: 38;
wherein the heavy chain variable domain comprises SEQ ID NO: 39 and the light chain variable domain comprises SEQ ID NO: 40;
wherein the heavy chain variable domain comprises SEQ ID NO: 43 and the light chain variable domain comprises SEQ ID NO: 44; or
wherein the heavy chain variable domain comprises SEQ ID NO: 45 and the light chain variable domain comprises SEQ ID NO: 46.

24-43. (canceled)

44. The antibody of claim 1, wherein the antibody neutralizes a coronavirus.

45. The antibody of claim 44, wherein the coronavirus is a SARS-CoV-2 wild type virus, SARS-CoV-2 WA1 variant, SARS-CoV-2 B.1.1.7 variant, SARS-CoV-2 B1.526 variant, SARS-CoV-2 B1.351 variant, SARS-CoV-2 P1 variant, SARS-CoV-2 B1.352 variant, or SARS-CoV-2 B.1.427 variant.

46-52. (canceled)

53. A method of treating or preventing a COVID-19 infection in a subject in need thereof, the method comprising administering to the subject a therapeutically effective amount of a synthesized monoclonal antibody, or a functional fragment thereof, comprising a heavy chain having a variable domain and a constant domain and a light chain having a variable domain and a constant domain, wherein the antibody binds an antigen on a coronavirus particle.

54-59. (canceled)

60. The method of claim 53, wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 19 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 20;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 21 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 22;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 25 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 26;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 29 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 30;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 31 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 32;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 33 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 34;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 37 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 38;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 39 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 40;
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 43 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 44; or
wherein the heavy chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 45 and the light chain variable domain comprises a polypeptide sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 46.

61-74. (canceled)

75. The method of claim 53, wherein the heavy chain variable domain comprises SEQ ID NO: 3 and the light chain variable domain comprises SEQ ID NO:
wherein the heavy chain variable domain comprises SEQ ID NO: 7 and the light chain variable domain comprises SEQ ID NO: 8;
wherein the heavy chain variable domain comprises SEQ ID NO: 9 and the light chain variable domain comprises SEQ ID NO: 10;
wherein the heavy chain variable domain comprises SEQ ID NO: 11 and the light chain variable domain comprises SEQ ID NO: 12;
wherein the heavy chain variable domain comprises SEQ ID NO: 17 and the light chain variable domain comprises SEQ ID NO: 18;
wherein the heavy chain variable domain comprises SEQ ID NO: 19 and the light chain variable domain comprises SEQ ID NO: 20;
wherein the heavy chain variable domain comprises SEQ ID NO: 21 and the light chain variable domain comprises SEQ ID NO: 22;
wherein the heavy chain variable domain comprises SEQ ID NO: 25 and the light chain variable domain comprises SEQ ID NO: 26;
wherein the heavy chain variable domain comprises SEQ ID NO: 29 and the light chain variable domain comprises SEQ ID NO: 30;
wherein the heavy chain variable domain comprises SEQ ID NO: 31 and the light chain variable domain comprises SEQ ID NO: 32;
wherein the heavy chain variable domain comprises SEQ ID NO: 33 and the light chain variable domain comprises SEQ ID NO: 34;
wherein the heavy chain variable domain comprises SEQ ID NO: 37 and the light chain variable domain comprises SEQ ID NO: 38;
wherein the heavy chain variable domain comprises SEQ ID NO: 39 and the light chain variable domain comprises SEQ ID NO: 40;
wherein the heavy chain variable domain comprises SEQ ID NO: 43 and the light chain variable domain comprises SEQ ID NO: 44; or
wherein the heavy chain variable domain comprises SEQ ID NO: 45 and the light chain variable domain comprises SEQ ID NO: 46.

76-95. (canceled)

96. The method of claim 53, wherein the antibody neutralizes a coronavirus.

97. The method of claim 96, wherein the coronavirus is a SARS-CoV-2 wild type virus, SARS-CoV-2 WA1 variant, SARS-CoV-2 B.1.1.7 variant, SARS-CoV-2 B1.526 variant, SARS-CoV-2 B1.351 variant, SARS-CoV-2 P1 variant, SARS-CoV-2 B1.352 variant, or SARS-CoV-2 B.1.427 variant.

98-105. (canceled)

106. A bispecific molecule comprising a first polypeptide chain and a second polypeptide chain, wherein:
the first polypeptide chain comprises:
a first light chain comprising a variable domain and a constant domain;
a first heavy chain comprising a variable domain and a constant domain;
the second polypeptide chain comprises:
a second light chain comprising a variable domain and a constant domain; and
a second heavy chain comprising a variable domain and a constant domain.

107. The molecule of claim 106, wherein the first polypeptide chain and the second polypeptide chain are covalently bonded to one another.

108-119. (canceled)

120. The molecule of claim 106, wherein the molecule comprises a polypeptide sequence which is at least 60% identical to SEQ ID NO: 51, a polypeptide sequence which is at least 60% identical to SEQ ID NO: 52, a polypeptide sequence which is at least 60% identical to SEQ ID NO: 53, and a polypeptide sequence which is at least 60% identical to SEQ ID NO: 54;
wherein the molecule comprises a polypeptide sequence which is at least 60% identical to SEQ ID NO: 55, a polypeptide sequence which is at least 60% identical to SEQ ID NO: 56, a polypeptide sequence which is at least 60% identical to SEQ ID NO: 57, and a polypeptide sequence which is at least 60% identical to SEQ ID NO: 58;
wherein the molecule comprises a polypeptide sequence which is at least 60% identical to SEQ ID NO: 67, a polypeptide sequence which is at least 60% identical to SEQ ID NO: 68, a polypeptide sequence which is at least 60% identical to SEQ ID NO: 69, and a polypeptide sequence which is at least 60% identical to SEQ ID NO: 70;
wherein the molecule comprises a polypeptide sequence which is at least 60% identical to SEQ ID NO: 71, a polypeptide sequence which is at least 60% identical to SEQ ID NO: 72, a polypeptide sequence which is at least 60% identical to SEQ ID NO: 73, and a polypeptide sequence which is at least 60% identical to SEQ ID NO: 74;
wherein the molecule comprises a polypeptide sequence which is at least 60% identical to SEQ ID NO: 75, a polypeptide sequence which is at least 60% identical to SEQ ID NO: 76, a polypeptide sequence which is at least 60% identical to SEQ ID NO: 77, and a polypeptide sequence which is at least 60% identical to SEQ ID NO: 78;
wherein the molecule comprises a polypeptide sequence which is at least 60% identical to SEQ ID NO: 79, a polypeptide sequence which is at least 60% identical to SEQ ID NO: 80, a polypeptide sequence which is at least 60% identical to SEQ ID NO: 81, a polypeptide sequence which is at least 60% identical to and SEQ ID NO: 82;
wherein the molecule comprises a polypeptide sequence which is at least 60% identical to SEQ ID NO: 83, a polypeptide sequence which is at least 60% identical to SEQ ID NO: 84, a polypeptide sequence which is at least 60% identical to SEQ ID NO: 85, and a polypeptide sequence which is at least 60% identical to SEQ ID NO: 86;
wherein the molecule comprises a polypeptide sequence which is at least 60% identical to SEQ ID NO: 87, a polypeptide sequence which is at least 60% identical to SEQ ID NO: 88, a polypeptide sequence which is at least 60% identical to SEQ ID NO: 89, and a polypeptide sequence which is at least 60% identical to SEQ ID NO: 90;
wherein the molecule comprises a polypeptide sequence which is at least 60% identical to SEQ ID NO: 91, a polypeptide sequence which is at least 60% identical to SEQ ID NO: 92, a polypeptide sequence which is at least 60% identical to SEQ ID NO: 93, and a polypeptide sequence which is at least 60% identical to SEQ ID NO: 94;
wherein the molecule comprises a polypeptide sequence which is at least 60% identical to SEQ ID NO: 95, a polypeptide sequence which is at least 60% identical to SEQ ID NO: 96, a polypeptide sequence which is at least 60% identical to SEQ ID NO: 97, and a polypeptide sequence which is at least 60% identical to SEQ ID NO: 98;
wherein the molecule comprises a polypeptide sequence which is at least 60% identical to SEQ ID NO: 99, a polypeptide sequence which is at least 60% identical to SEQ ID NO: 100, a polypeptide sequence which is at least 60% identical to SEQ ID NO: 101, and a polypeptide sequence which is at least 60% identical to SEQ ID NO: 102;
wherein the molecule comprises a polypeptide sequence which is at least 60% identical to SEQ ID NO: 103, a polypeptide sequence which is at least 60% identical to SEQ ID NO: 104, a polypeptide sequence which is at least 60% identical to SEQ ID NO: 105, and a polypeptide sequence which is at least 60% identical to SEQ ID NO: 106;
wherein the molecule comprises a polypeptide sequence which is at least 60% identical to SEQ ID NO: 107, a polypeptide sequence which is at least 60% identical to SEQ ID NO: 108, a polypeptide sequence which is at least 60% identical to SEQ ID NO: 109, and a polypeptide sequence which is at least 60% identical to SEQ ID NO: 110;
wherein the molecule comprises a polypeptide sequence which is at least 60% identical to SEQ ID NO: 115, a polypeptide sequence which is at least 60% identical to SEQ ID NO: 116, a polypeptide sequence which is at least 60% identical to SEQ ID NO: 117, and a polypeptide sequence which is at least 60% identical to SEQ ID NO: 118;
wherein the molecule comprises a polypeptide sequence which is at least 60% identical to SEQ ID NO: 119, a polypeptide sequence which is at least 60% identical to SEQ ID NO: 120, a polypeptide sequence which is at least 60% identical to SEQ ID NO: 121, and a polypeptide sequence which is at least 60% identical to SEQ ID NO: 122;
wherein the molecule comprises a polypeptide sequence which is at least 60% identical to SEQ ID NO: 123, a polypeptide sequence which is at least 60% identical to SEQ ID NO: 124, a polypeptide sequence which is at least 60% identical to SEQ ID NO: 125, and a polypeptide sequence which is at least 60% identical to SEQ ID NO: 126;
wherein the molecule comprises a polypeptide sequence which is at least 60% identical to SEQ ID NO: 127, a polypeptide sequence which is at least 60% identical to SEQ ID NO: 128, a polypeptide sequence which is at least 60% identical to SEQ ID NO: 129, and a polypeptide sequence which is at least 60% identical to SEQ ID NO: 130;
wherein the molecule comprises a polypeptide sequence which is at least 60% identical to SEQ ID NO: 131, a polypeptide sequence which is at least 60% identical to SEQ ID NO: 132, a polypeptide sequence which is at least 60% identical to SEQ ID NO: 133, and a polypeptide sequence which is at least 60% identical to SEQ ID NO: 133;
wherein the molecule comprises a polypeptide sequence which is at least 60% identical to SEQ ID NO: 139, a polypeptide sequence which is at least 60% identical to SEQ ID NO: 140, a polypeptide sequence which is at least 60% identical to SEQ ID NO: 141, and a polypeptide sequence which is at least 60% identical to SEQ ID NO: 142;
wherein the molecule comprises a polypeptide sequence which is at least 60% identical to SEQ ID NO: 143, a polypeptide sequence which is at least 60% identical to SEQ ID NO: 144, a polypeptide sequence which is at least 60% identical to SEQ ID NO: 145, and a polypeptide sequence which is at least 60% identical to SEQ ID NO: 146;
wherein the molecule comprises a polypeptide sequence which is at least 60% identical to SEQ ID NO: 147, a polypeptide sequence which is at least 60% identical to SEQ ID NO: 148, a polypeptide sequence which is at least 60% identical to SEQ ID NO: 149, and a polypeptide sequence which is at least 60% identical to SEQ ID NO: 150;
wherein the molecule comprises a polypeptide sequence which is at least 60% identical to SEQ ID NO: 151, a polypeptide sequence which is at least 60% identical to SEQ ID NO: 152, a polypeptide sequence which is at least 60% identical to SEQ ID NO: 153, and a polypeptide sequence which is at least 60% identical to SEQ ID NO: 154;
wherein the molecule comprises a polypeptide sequence which is at least 60% identical to SEQ ID NO: 155, a polypeptide sequence which is at least 60% identical to SEQ ID NO: 156, a polypeptide sequence which is at least 60% identical to SEQ ID NO: 157, and a polypeptide sequence which is at least 60% identical to SEQ ID NO: 158;
wherein the molecule comprises a polypeptide sequence which is at least 60% identical to SEQ ID NO: 159, a polypeptide sequence which is at least 60% identical to SEQ ID NO: 160, a polypeptide sequence which is at least 60% identical to SEQ ID NO: 161, and a polypeptide sequence which is at least 60% identical to SEQ ID NO: 162;
wherein the molecule comprises a polypeptide sequence which is at least 60% identical to SEQ ID NO: 163, a polypeptide sequence which is at least 60% identical to SEQ ID NO: 164, a polypeptide sequence which is at least 60% identical to SEQ ID NO: 165, and a polypeptide sequence which is at least 60% identical to SEQ ID NO: 166;
wherein the molecule comprises a polypeptide sequence which is at least 60% identical to SEQ ID NO: 167, a polypeptide sequence which is at least 60% identical to SEQ ID NO: 168, a polypeptide sequence which is at least 60% identical to SEQ ID NO: 169, and a polypeptide sequence which is at least 60% identical to SEQ ID NO: 170;
wherein the molecule comprises a polypeptide sequence which is at least 60% identical to SEQ ID NO: 171, a polypeptide sequence which is at least 60% identical to SEQ ID NO: 172, a polypeptide sequence which is at least 60% identical to SEQ ID NO: 173, and a polypeptide sequence which is at least 60% identical to SEQ ID NO: 174;
wherein the molecule comprises a polypeptide sequence which is at least 60% identical to SEQ ID NO: 175, a polypeptide sequence which is at least 60% identical to SEQ ID NO: 176, a polypeptide sequence which is at least 60% identical to SEQ ID NO: 177, and a polypeptide sequence which is at least 60% identical to SEQ ID NO: 178;
wherein the molecule comprises a polypeptide sequence which is at least 60% identical to SEQ ID NO: 179, a polypeptide sequence which is at least 60% identical to SEQ ID NO: 180, a polypeptide sequence which is at least 60% identical to SEQ ID NO: 181, and a polypeptide sequence which is at least 60% identical to SEQ ID NO: 182; or
wherein the molecule comprises a polypeptide sequence which is at least 60% identical to SEQ ID NO: 183, a polypeptide sequence which is at least 60% identical to SEQ ID NO: 184, a polypeptide sequence which is at least 60% identical to SEQ ID NO: 185, and a polypeptide sequence which is at least 60% identical to SEQ ID NO: 186.

121. The molecule of claim 106, wherein the molecule comprises SEQ ID NO: 55, SEQ ID NO: 56, SEQ ID NO: 57, and SEQ ID NO: 58;
wherein the molecule comprises SEQ ID NO: 67, SEQ ID NO: 68, SEQ ID NO: 69, and SEQ ID NO: 70;
wherein the molecule comprises SEQ ID NO: 71, SEQ ID NO: 72, SEQ ID NO: 73, and SEQ ID NO: 74;
wherein the molecule comprises SEQ ID NO: 75, SEQ ID NO: 76, SEQ ID NO: 77, and SEQ ID NO: 78;
wherein the molecule comprises SEQ ID NO: 79, SEQ ID NO: 80, SEQ ID NO: 81, and SEQ ID NO: 82;
wherein the molecule comprises SEQ ID NO: 83, SEQ ID NO: 84, SEQ ID NO: 85, and SEQ ID NO: 86;
wherein the molecule comprises SEQ ID NO: 87, SEQ ID NO: 88, SEQ ID NO: 89, and SEQ ID NO: 90;
wherein the molecule comprises SEQ ID NO: 91, SEQ ID NO: 92, SEQ ID NO: 93, and SEQ ID NO: 94;
wherein the molecule comprises SEQ ID NO: 95, SEQ ID NO: 96, SEQ ID NO: 97, and SEQ ID NO: 98;
wherein the molecule comprises SEQ ID NO: 99, SEQ ID NO: 100, SEQ ID NO: 101, and SEQ ID NO: 102;
wherein the molecule comprises SEQ ID NO: 103, SEQ ID NO: 104, SEQ ID NO: 105, and SEQ ID NO: 106;
wherein the molecule comprises SEQ ID NO: 107, SEQ ID NO: 108, SEQ ID NO: 109, and SEQ ID NO: 110;
wherein the molecule comprises SEQ ID NO: 115, SEQ ID NO: 116, SEQ ID NO: 117, and SEQ ID NO: 118;
wherein the molecule comprises SEQ ID NO: 119, SEQ ID NO: 120, SEQ ID NO: 121, and SEQ ID NO: 122;
wherein the molecule comprises SEQ ID NO: 123, SEQ ID NO: 124, SEQ ID NO: 125, and SEQ ID NO: 126;
wherein the molecule comprises SEQ ID NO: 127, SEQ ID NO: 128, SEQ ID NO: 129, and SEQ ID NO: 130;
wherein the molecule comprises SEQ ID NO: 131, SEQ ID NO: 132, SEQ ID NO: 133, and SEQ ID NO: 133;
wherein the molecule comprises SEQ ID NO: 139, SEQ ID NO: 140, SEQ ID NO: 141, and SEQ ID NO: 142;
wherein the molecule comprises SEQ ID NO: 143, SEQ ID NO: 144, SEQ ID NO: 145, and SEQ ID NO: 146;
wherein the molecule comprises SEQ ID NO: 147, SEQ ID NO: 148, SEQ ID NO: 149, and SEQ ID NO: 150;
wherein the molecule comprises SEQ ID NO: 151, SEQ ID NO: 152, SEQ ID NO: 153, and SEQ ID NO: 154;
wherein the molecule comprises SEQ ID NO: 155, SEQ ID NO: 156, SEQ ID NO: 157, and SEQ ID NO: 158;
wherein the molecule comprises SEQ ID NO: 159, SEQ ID NO: 160, SEQ ID NO: 161, and SEQ ID NO: 162;
wherein the molecule comprises SEQ ID NO: 163, SEQ ID NO: 164, SEQ ID NO: 165, and SEQ ID NO: 166;
wherein the molecule comprises SEQ ID NO: 167, SEQ ID NO: 168, SEQ ID NO: 169, and SEQ ID NO: 170;
wherein the molecule comprises SEQ ID NO: 171, SEQ ID NO: 172, SEQ ID NO: 173, and SEQ ID NO: 174;
wherein the molecule comprises SEQ ID NO: 175, SEQ ID NO: 176, SEQ ID NO: 177, and SEQ ID NO: 178;
wherein the molecule comprises SEQ ID NO: 179, SEQ ID NO: 180, SEQ ID NO: 181, and SEQ ID NO: 182; or
wherein the molecule comprises SEQ ID NO: 183, SEQ ID NO: 184, SEQ ID NO: 185, and SEQ ID NO: 186.

122-149. (canceled)

150. The molecule of claim 106, wherein the molecule further comprises one or more disulfide bonds.

151. The molecule of claim 106, wherein the molecule further comprises one or more linker sequences.

152. The molecule of claim 106, wherein the molecule is capable of neutralizing a coronavirus.

153. The molecule of claim 152, wherein the coronavirus is a SARS-CoV-2 wild type virus, SARS-CoV-2 WA1 variant, SARS-CoV-2 B.1.1.7 variant, SARS-CoV-2 B1.526 variant, SARS-CoV-2 B1.351 variant, SARS-CoV-2 P1 variant, SARS-CoV-2 B1.352 variant, or SARS-CoV-2 B.1.427 variant.

154-157. (canceled)

158. A method of treating or preventing a COVID-19 infection in a subject in need thereof, the method comprising administering to the subject a composition comprising a bispecific molecule comprising a first polypeptide chain and a second polypeptide chain, wherein:
the first polypeptide chain comprises:
a first light chain comprising a variable domain and a constant domain;
a first heavy chain comprising a variable domain and a constant domain;
the second polypeptide chain comprises:
a second light chain comprising a variable domain and a constant domain; and
a second heavy chain comprising a variable domain and a constant domain.

159. The method of claim 158, wherein the first polypeptide chain and the second polypeptide chain are covalently bonded to one another.

160-171. (canceled)

172. The method of claim 158, wherein the molecule comprises a polypeptide sequence which is at least 60% identical to SEQ ID NO: 51, a polypeptide sequence which is at least 60% identical to SEQ ID NO: 52, a polypeptide sequence which is at least 60% identical to SEQ ID NO: 53, and a polypeptide sequence which is at least 60% identical to SEQ ID NO: 54;
wherein the molecule comprises a polypeptide sequence which is at least 60% identical to SEQ ID NO: 55, a polypeptide sequence which is at least 60% identical to SEQ ID NO: 56, a polypeptide sequence which is at least 60% identical to SEQ ID NO: 57, and a polypeptide sequence which is at least 60% identical to SEQ ID NO: 58;
wherein the molecule comprises a polypeptide sequence which is at least 60% identical to SEQ ID NO: 67, a polypeptide sequence which is at least 60% identical to SEQ ID NO: 68, a polypeptide sequence which is at least 60% identical to SEQ ID NO: 69, and a polypeptide sequence which is at least 60% identical to SEQ ID NO: 70;
wherein the molecule comprises a polypeptide sequence which is at least 60% identical to SEQ ID NO: 71, a polypeptide sequence which is at least 60% identical to SEQ ID NO: 72, a polypeptide sequence which is at least 60% identical to SEQ ID NO: 73, and a polypeptide sequence which is at least 60% identical to SEQ ID NO: 74;
wherein the molecule comprises a polypeptide sequence which is at least 60% identical to SEQ ID NO: 75, a polypeptide sequence which is at least 60% identical to SEQ ID NO: 76, a polypeptide sequence which is at least 60% identical to SEQ ID NO: 77, and a polypeptide sequence which is at least 60% identical to SEQ ID NO: 78;
wherein the molecule comprises a polypeptide sequence which is at least 60% identical to SEQ ID NO: 79, a polypeptide sequence which is at least 60% identical to SEQ ID NO: 80, a polypeptide sequence which is at least 60% identical to SEQ ID NO: 81, a polypeptide sequence which is at least 60% identical to and SEQ ID NO: 82;
wherein the molecule comprises a polypeptide sequence which is at least 60% identical to SEQ ID NO: 83, a polypeptide sequence which is at least 60% identical to SEQ ID NO: 84, a polypeptide sequence which is at least 60% identical to SEQ ID NO: 85, and a polypeptide sequence which is at least 60% identical to SEQ ID NO: 86;
wherein the molecule comprises a polypeptide sequence which is at least 60% identical to SEQ ID NO: 87, a polypeptide sequence which is at least 60% identical to SEQ ID NO: 88, a polypeptide sequence which is at least 60% identical to SEQ ID NO: 89, and a polypeptide sequence which is at least 60% identical to SEQ ID NO: 90;
wherein the molecule comprises a polypeptide sequence which is at least 60% identical to SEQ ID NO: 91, a polypeptide sequence which is at least 60% identical to SEQ ID NO: 92, a polypeptide sequence which is at least 60% identical to SEQ ID NO: 93, and a polypeptide sequence which is at least 60% identical to SEQ ID NO: 94;
wherein the molecule comprises a polypeptide sequence which is at least 60% identical to SEQ ID NO: 95, a polypeptide sequence which is at least 60% identical to SEQ ID NO: 96, a polypeptide sequence which is at least 60% identical to SEQ ID NO: 97, and a polypeptide sequence which is at least 60% identical to SEQ ID NO: 98;
wherein the molecule comprises a polypeptide sequence which is at least 60% identical to SEQ ID NO: 99, a polypeptide sequence which is at least 60% identical to SEQ ID NO: 100, a polypeptide sequence which is at least 60% identical to SEQ ID NO: 101, and a polypeptide sequence which is at least 60% identical to SEQ ID NO: 102;
wherein the molecule comprises a polypeptide sequence which is at least 60% identical to SEQ ID NO: 103, a polypeptide sequence which is at least 60% identical to SEQ ID NO: 104, a polypeptide sequence which is at least 60% identical to SEQ ID NO: 105, and a polypeptide sequence which is at least 60% identical to SEQ ID NO: 106;
wherein the molecule comprises a polypeptide sequence which is at least 60% identical to SEQ ID NO: 107, a polypeptide sequence which is at least 60% identical to SEQ ID NO: 108, a polypeptide sequence which is at least 60% identical to SEQ ID NO: 109, and a polypeptide sequence which is at least 60% identical to SEQ ID NO: 110;
wherein the molecule comprises a polypeptide sequence which is at least 60% identical to SEQ ID NO: 115, a polypeptide sequence which is at least 60% identical to SEQ ID NO: 116, a polypeptide sequence which is at least 60% identical to SEQ ID NO: 117, and a polypeptide sequence which is at least 60% identical to SEQ ID NO: 118;
wherein the molecule comprises a polypeptide sequence which is at least 60% identical to SEQ ID NO: 119, a polypeptide sequence which is at least 60% identical to SEQ ID NO: 120, a polypeptide sequence which is at least 60% identical to SEQ ID NO: 121, and a polypeptide sequence which is at least 60% identical to SEQ ID NO: 122;
wherein the molecule comprises a polypeptide sequence which is at least 60% identical to SEQ ID NO: 123, a polypeptide sequence which is at least 60% identical to SEQ ID NO: 124, a polypeptide sequence which is at least 60% identical to SEQ ID NO: 125, and a polypeptide sequence which is at least 60% identical to SEQ ID NO: 126;
wherein the molecule comprises a polypeptide sequence which is at least 60% identical to SEQ ID NO: 127, a polypeptide sequence which is at least 60% identical to SEQ ID NO: 128, a polypeptide sequence which is at least 60% identical to SEQ ID NO: 129, and a polypeptide sequence which is at least 60% identical to SEQ ID NO: 130;
wherein the molecule comprises a polypeptide sequence which is at least 60% identical to SEQ ID NO: 131, a polypeptide sequence which is at least 60% identical to SEQ ID NO: 132, a polypeptide sequence which is at least 60% identical to SEQ ID NO: 133, and a polypeptide sequence which is at least 60% identical to SEQ ID NO: 133;
wherein the molecule comprises a polypeptide sequence which is at least 60% identical to SEQ ID NO: 139, a polypeptide sequence which is at least 60% identical to SEQ ID NO: 140, a polypeptide sequence which is at least 60% identical to SEQ ID NO: 141, and a polypeptide sequence which is at least 60% identical to SEQ ID NO: 142;
wherein the molecule comprises a polypeptide sequence which is at least 60% identical to SEQ ID NO: 143, a polypeptide sequence which is at least 60% identical to SEQ ID NO: 144, a polypeptide sequence which is at least 60% identical to SEQ ID NO: 145, and a polypeptide sequence which is at least 60% identical to SEQ ID NO: 146;
wherein the molecule comprises a polypeptide sequence which is at least 60% identical to SEQ ID NO: 147, a polypeptide sequence which is at least 60% identical to SEQ ID NO: 148, a polypeptide sequence which is at least 60% identical to SEQ ID NO: 149, and a polypeptide sequence which is at least 60% identical to SEQ ID NO: 150;
wherein the molecule comprises a polypeptide sequence which is at least 60% identical to SEQ ID NO: 151, a polypeptide sequence which is at least 60% identical to SEQ ID NO: 152, a polypeptide sequence which is at least 60% identical to SEQ ID NO: 153, and a polypeptide sequence which is at least 60% identical to SEQ ID NO: 154;
wherein the molecule comprises a polypeptide sequence which is at least 60% identical to SEQ ID NO: 155, a polypeptide sequence which is at least 60% identical to SEQ ID NO: 156, a polypeptide sequence which is at least 60% identical to SEQ ID NO: 157, and a polypeptide sequence which is at least 60% identical to SEQ ID NO: 158;
wherein the molecule comprises a polypeptide sequence which is at least 60% identical to SEQ ID NO: 159, a polypeptide sequence which is at least 60% identical to SEQ ID NO: 160, a polypeptide sequence which is at least 60% identical to SEQ ID NO: 161, and a polypeptide sequence which is at least 60% identical to SEQ ID NO: 162;
wherein the molecule comprises a polypeptide sequence which is at least 60% identical to SEQ ID NO: 163, a polypeptide sequence which is at least 60% identical to SEQ ID NO: 164, a polypeptide sequence which is at least 60% identical to SEQ ID NO: 165, and a polypeptide sequence which is at least 60% identical to SEQ ID NO: 166;
wherein the molecule comprises a polypeptide sequence which is at least 60% identical to SEQ ID NO: 167, a polypeptide sequence which is at least 60% identical to SEQ ID NO: 168, a polypeptide sequence which is at least 60% identical to SEQ ID NO: 169, and a polypeptide sequence which is at least 60% identical to SEQ ID NO: 170;
wherein the molecule comprises a polypeptide sequence which is at least 60% identical to SEQ ID NO: 171, a polypeptide sequence which is at least 60% identical to SEQ ID NO: 172, a polypeptide sequence which is at least 60% identical to SEQ ID NO: 173, and a polypeptide sequence which is at least 60% identical to SEQ ID NO: 174;
wherein the molecule comprises a polypeptide sequence which is at least 60% identical to SEQ ID NO: 175, a polypeptide sequence which is at least 60% identical to SEQ ID NO: 176, a polypeptide sequence which is at least 60% identical to SEQ ID NO: 177, and a polypeptide sequence which is at least 60% identical to SEQ ID NO: 178;
wherein the molecule comprises a polypeptide sequence which is at least 60% identical to SEQ ID NO: 179, a polypeptide sequence which is at least 60% identical to SEQ ID NO: 180, a polypeptide sequence which is at least 60% identical to SEQ ID NO: 181, and a polypeptide sequence which is at least 60% identical to SEQ ID NO: 182; or
wherein the molecule comprises a polypeptide sequence which is at least 60% identical to SEQ ID NO: 183, a polypeptide sequence which is at least 60% identical to SEQ ID NO: 184, a polypeptide sequence which is at least 60% identical to SEQ ID NO: 185, and a polypeptide sequence which is at least 60% identical to SEQ ID NO: 186.

173. The method of claim 158, wherein the molecule comprises SEQ ID NO: 55, SEQ ID NO: 56, SEQ ID NO: 57, and SEQ ID NO: 58;
wherein the molecule comprises SEQ ID NO: 67, SEQ ID NO: 68, SEQ ID NO: 69, and SEQ ID NO: 70;
wherein the molecule comprises SEQ ID NO: 71, SEQ ID NO: 72, SEQ ID NO: 73, and SEQ ID NO: 74;
wherein the molecule comprises SEQ ID NO: 75, SEQ ID NO: 76, SEQ ID NO: 77, and SEQ ID NO: 78;
wherein the molecule comprises SEQ ID NO: 79, SEQ ID NO: 80, SEQ ID NO: 81, and SEQ ID NO: 82;
wherein the molecule comprises SEQ ID NO: 83, SEQ ID NO: 84, SEQ ID NO: 85, and SEQ ID NO: 86;
wherein the molecule comprises SEQ ID NO: 87, SEQ ID NO: 88, SEQ ID NO: 89, and SEQ ID NO: 90;
wherein the molecule comprises SEQ ID NO: 91, SEQ ID NO: 92, SEQ ID NO: 93, and SEQ ID NO: 94;
wherein the molecule comprises SEQ ID NO: 95, SEQ ID NO: 96, SEQ ID NO: 97, and SEQ ID NO: 98;
wherein the molecule comprises SEQ ID NO: 99, SEQ ID NO: 100, SEQ ID NO: 101, and SEQ ID NO: 102;
wherein the molecule comprises SEQ ID NO: 103, SEQ ID NO: 104, SEQ ID NO: 105, and SEQ ID NO: 106;
wherein the molecule comprises SEQ ID NO: 107, SEQ ID NO: 108, SEQ ID NO: 109, and SEQ ID NO: 110;
wherein the molecule comprises SEQ ID NO: 115, SEQ ID NO: 116, SEQ ID NO: 117, and SEQ ID NO: 118;
wherein the molecule comprises SEQ ID NO: 119, SEQ ID NO: 120, SEQ ID NO: 121, and SEQ ID NO: 122;
wherein the molecule comprises SEQ ID NO: 123, SEQ ID NO: 124, SEQ ID NO: 125, and SEQ ID NO: 126;
wherein the molecule comprises SEQ ID NO: 127, SEQ ID NO: 128, SEQ ID NO: 129, and SEQ ID NO: 130;
wherein the molecule comprises SEQ ID NO: 131, SEQ ID NO: 132, SEQ ID NO: 133, and SEQ ID NO: 133;
wherein the molecule comprises SEQ ID NO: 139, SEQ ID NO: 140, SEQ ID NO: 141, and SEQ ID NO: 142;
wherein the molecule comprises SEQ ID NO: 143, SEQ ID NO: 144, SEQ ID NO: 145, and SEQ ID NO: 146;
wherein the molecule comprises SEQ ID NO: 147, SEQ ID NO: 148, SEQ ID NO: 149, and SEQ ID NO: 150;
wherein the molecule comprises SEQ ID NO: 151, SEQ ID NO: 152, SEQ ID NO: 153, and SEQ ID NO: 154;
wherein the molecule comprises SEQ ID NO: 155, SEQ ID NO: 156, SEQ ID NO: 157, and SEQ ID NO: 158;
wherein the molecule comprises SEQ ID NO: 159, SEQ ID NO: 160, SEQ ID NO: 161, and SEQ ID NO: 162;
wherein the molecule comprises SEQ ID NO: 163, SEQ ID NO: 164, SEQ ID NO: 165, and SEQ ID NO: 166;
wherein the molecule comprises SEQ ID NO: 167, SEQ ID NO: 168, SEQ ID NO: 169, and SEQ ID NO: 170;
wherein the molecule comprises SEQ ID NO: 171, SEQ ID NO: 172, SEQ ID NO: 173, and SEQ ID NO: 174;
wherein the molecule comprises SEQ ID NO: 175, SEQ ID NO: 176, SEQ ID NO: 177, and SEQ ID NO: 178;
wherein the molecule comprises SEQ ID NO: 179, SEQ ID NO: 180, SEQ ID NO: 181, and SEQ ID NO: 182; or
wherein the molecule comprises SEQ ID NO: 183, SEQ ID NO: 184, SEQ ID NO: 185, and SEQ ID NO: 186.

174-201. (canceled)

202. The method of claim 158, wherein the molecule further comprises one or more disulfide bonds.

203. The method of claim 158, wherein the molecule further comprises one or more linker sequences.

204. The method of claim 158, wherein the molecule is capable of neutralizing a coronavirus.

205. The method of claim 204, wherein the coronavirus is a SARS-CoV-2 wild type virus, SARS-CoV-2 WA1 variant, SARS-CoV-2 B.1.1.7 variant, SARS-CoV-2 B1.526 variant, SARS-CoV-2 B1.351 variant, SARS-CoV-2 P1 variant, SARS-CoV-2 B1.352 variant, or SARS-CoV-2 B.1.427 variant.

206-210. (canceled)
